(19) United States
(12) Patent Application Publication
(10) Pub. No.: US 2024/0043439 A1
(43) Pub. Date: Feb. 8, 2024

(54) COMPOUNDS AS C5AR INHIBITORS

(71) Applicant: Kira Pharmaceuticals (Suzhou) Ltd., Suzhou, Jiangsu (CN)

(72) Inventors: Gonghua PAN, Suzhou, Jiangsu (CN); Xihua ZHU, Suzhou, Jiangsu (CN); Yingjie ZHU, Suzhou, Jiangsu (CN)

(73) Assignee: Kira Pharmaceuticals (Suzhou) Ltd., Suzhou, Jiangsu (CN)

(21) Appl. No.: 18/020,242

(22) PCT Filed: Aug. 6, 2021
(86) PCT No.: PCT/CN2021/111236

§ 371 Date: Feb. 7, 2023

(30) Foreign Application Priority Data
Aug. 7, 2020 (WO) PCT/CN2020/107800

(51) Int. Cl. C07D 495/04; C07D 401/04; C07D 471/04; C07D 211/96; C07D 401/12; C07D 413/12; C07D 211/60; C07D 405/12; C07D 405/04; C07D 405/14; C07D 417/14; C07D 401/06; C07D 401/14; C07F 9/576; C07D 405/06; C07D 411/06; C07D 491/048; C07D 519/00; C07D 265/30; A61P 37/02; C07F 9/6561

(52) U.S. Cl. CPC C07D 495/04; C07D 401/04; C07D 471/04; C07D 211/96; C07D 401/12; C07D 413/12; C07D 211/60; C07D 405/12; C07D 405/04; C07D 405/14; C07D 417/14; C07D 401/06; C07D 401/14; C07F 9/5765; C07D 405/06; C07D 411/06; C07D 491/048; C07D 519/00; C07D 265/30; A61P 37/02; C07F 9/6561

(57) ABSTRACT

Disclosed generally relates to C5a receptor inhibitors, compositions thereof, methods of use thereof, and methods of preparation thereof.

### CROSS-REFERENCE TO RELATED APPLICATION

This application claims priority benefit of PCT International Application No. PCT/CN2020/107800, filed Aug. 7, 2020, the disclosure of which is hereby incorporated herein by reference in its entirety.

### FIELD

The present disclosure relates to C5a receptor inhibitors, compositions thereof, methods of use thereof, and methods of preparation thereof.

### BACKGROUND

C5a is a 74 amino acid peptide that is generated by the proteolysis of complement protein C5. Increased level of C5a has been associated with disorders such as autoimmune disorders and inflammatory disorders. The effects of C5a are believed to be mediated through its binding to the C5a receptor (C5aR). As such, there is a need for therapeutics that inhibit the activity of C5aR and thus inhibit the binding of C5a to C5aR. The present disclosure provides compounds that are C5aR inhibitors.

### BRIEF DESCRIPTION OF THE DRAWINGS

FIG. 1A shows the experimental design of the effect C5aR compounds have on C5a induced neutropenia in cyno monkeys.

FIG. 1B shows the in vivo rescue effect of compound Nos. 47 and 49 in human C5a induced neutropenia model in cyno monkeys.

FIG. 2A shows the experimental design of the effect C5aR compounds have on C5a induced neutropenia in human C5aR knocked-in mice.

FIG. 2B shows the in vivo rescue effect of compound No. 49 in human C5a induced neutropenia model in human C5aR knock-in mice.

FIG. 3 shows C5a induced CD11b upregulation on granulocytes in cyno monkey whole blood was blocked by orally pre-dosing compound Nos. 47 and 49 at 10 mg/kg.

FIG. 4 shows C5a induced CD11b upregulation on neutrophil was blocked by compound No. 49 on neutrophil in mice whole blood by orally pre-dosing.

FIG. 5 shows C5a induced CD11b upregulation on neutrophil was blocked by compound Nos. 47 and 89 on neutrophil in human C5aR knock-in mice whole blood by orally pre-dosing.

FIG. 6 shows C5a induced CD11b upregulation on neutrophil was blocked by compound Nos. 47 and 49 on neutrophil in human C5aR knock-in mice whole blood by orally pre-dosing.

### SUMMARY

In one aspect, provided is a compound of formula (I):

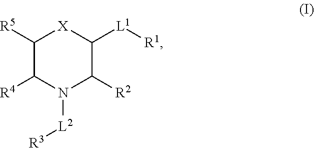

or a stereoisomer, tautomer, or a pharmaceutically acceptable salt of any of the foregoing, wherein R¹, R², R³, R⁴, R⁵, X, L¹, and L² are as disclosed herein.

In another aspect, provided is a pharmaceutical composition comprising a compound as described herein and a pharmaceutically acceptable carrier or excipient. Also provided is a kit comprising a compound as described herein.

In another aspect, provided is a method of treating a C5a-mediated disorder in an individual in need thereof, comprising administering an therapeutically effective amount of a compound as described herein, or pharmaceutically acceptable salt thereof, to the individual. Also provided is use of a compound as described herein, or pharmaceutically acceptable salt thereof, in the manufacture of a medicament for the treatment of a C5a-mediated disease.

### DETAILED DESCRIPTION

The following description sets forth exemplary embodiments of the present disclosure. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

The term “about” refers to a variation of ±1%, ±3%, ±5%, or ±10% of the value specified. For example, “about 50” can in some embodiments includes a range of from 45 to 55. Reference to “about” a value or parameter herein includes (and describes) embodiments that are directed to that value or parameter per se. For example, description referring to “about X” includes description of “X”.

The singular forms “a,” “an,” and “the” include plural references unless the context clearly dictates otherwise. Thus, e.g., reference to “the compound” includes a plurality of such compounds and includes reference to one or more compounds and equivalents thereof known to those skilled in the art.

“Alkyl” refers to an unbranched or branched saturated hydrocarbon chain. As used herein, alkyl has 1 to 10 carbon atoms (i.e., C₁₋₁₀ alkyl or C₁-C₁₀ alkyl), 1 to 8 carbon atoms (i.e., C₁₋₈ alkyl or C₁-C₈ alkyl), 1 to 6 carbon atoms (i.e., C₁₋₆ alkyl or C₁-C₆ alkyl), or 1 to 4 carbon atoms (i.e., C₁₋₄ alkyl or C₁-C₄ alkyl). Examples of alkyl groups include, but are not limited to, methyl, ethyl, propyl, isopropyl, n-butyl, sec-butyl, isobutyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkylene” refers to a divalent alkyl group as defined herein.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NH—, —O—, —S—, —S(O)—, —S(O)₂— and the like. As used herein, heteroalkyl includes 1 to 8 carbon atoms, or 1 to 4 carbon atoms; and 1 to 3 heteroatomic groups, 1 to 2 heteroatomic groups, or 1 heteroatomic group.

“Heteroalkylene” refers to a divalent heteroalkyl group as defined herein.

“Alkoxy” refers to the group “—O-alkyl”. Examples of alkoxy groups include, but are not limited to, methoxy, ethoxy, n-propoxy, isopropoxy, n-butoxy, tert-butoxy, sec-butoxy, n-pentoxy, n-hexoxy and 1,2-dimethylbutoxy.

“Alkenyl” refers to an alkyl group containing at least one carbon-carbon double bond and having from 2 to 20 carbon atoms (i.e., C₂₋₂₀ alkenyl or C₂-C₂₀ alkenyl), 2 to 8 carbon atoms (i.e., C₂₋₈ alkenyl or C₂-C₈ alkenyl), 2 to 6 carbon atoms (i.e., C₂₋₆ alkenyl or C₂-C₆ alkenyl) or 2 to 4 carbon atoms (i.e., C₂₋₄ alkenyl or C₂-C₄ alkenyl). Examples of alkenyl groups include, but are not limited to, ethenyl, propenyl, and butadienyl (e.g., 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl or C₂-C₂₀ alkynyl), 2 to 8 carbon atoms (i.e., C₂₋₈ alkynyl or C₂-C₈ alkynyl), 2 to 6 carbon atoms (i.e., C₂₋₆ alkynyl or C₂-C₆ alkynyl) or 2 to 4 carbon atoms (i.e., C₂₋₄ alkynyl or C₂-C₄ alkynyl). The term “alkynyl” also includes those groups having one triple bond and one double bond.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl or C₆-C₂₀ aryl), 6 to 12 carbon ring atoms (i.e., C₆₋₁₂ aryl or C₆-C₁₂ aryl), or 6 to 10 carbon ring atoms (i.e., C₆₋₁₀ aryl or C₆-C₁₀ aryl). Examples of aryl groups include, but are not limited to, phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl or C₃-C₂₀ cycloalkyl), 3 to 12 ring carbon atoms (i.e., C₃₋₁₂ cycloalkyl or C₃-C₁₂ cycloalkyl), 3 to 10 ring carbon atoms (i.e., C₃₋₁₀ cycloalkyl or C₃-C10 cycloalkyl), 3 to 8 ring carbon atoms (i.e., C₃₋₈ cycloalkyl or C₃-C₈ cycloalkyl), or 3 to 6 ring carbon atoms (i.e., C₃₋₆ cycloalkyl or C₃-C₆ cycloalkyl). Monocyclic groups include, but are not limited to, cyclopropyl, cyclobutyl, cyclopentyl, cyclohexyl, cycloheptyl and cyclooctyl. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom.

“Heteroaryl” refers to an aromatic group having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl) and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5- to 14-membered ring systems, 5- to 12-membered ring systems, 5- to 10-membered ring systems, 5- to 7-membered ring systems, or 5- to 6-membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro and may comprise one or more (e.g., 1 to 3) oxo (═O) or N-oxide (N⁺—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ or C₂-C₂₀ heterocyclyl), 2 to 12 ring carbon atoms (i.e., C₂₋₁₂ or C₂-C₁₂ heterocyclyl), 2 to 10 ring carbon atoms (i.e., C₂₋₁₀ or C₂-C₁₀ heterocyclyl), 2 to 8 ring carbon atoms (i.e., C₂₋₈ or C₂-C₈ heterocyclyl), 3 to 12 ring carbon atoms (i.e., C₃₋₁₂ or C₃-C₁₂ heterocyclyl), 3 to 8 ring carbon atoms (i.e., C₃₋₈ or C₃-C₈ heterocyclyl), or 3 to 6 ring carbon atoms (i.e., C₃₋₆ or C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. In certain instances, heterocyclyl includes 3- to 14-membered ring systems, 3- to 12-membered ring systems, 5- to 10-membered ring systems, 5- to 7-membered ring systems, or 5- to 6-membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur.

“Oxo” refers to ═O.

“Halogen” or “halo” includes fluoro, chloro, bromo and iodo.

The terms “optional” or “optionally” means that the subsequently described event or circumstance may or may not occur. The term “optionally substituted” refers to any one or more (e.g., 1-5, 1-4, 1-3, 1-2, 2-5, 2-4, 2-3, 3-5, or 3-4) hydrogen atoms on the designated atom or group may or may not be replaced by a substituent atom or group commonly used in pharmaceutical chemistry. Each substituent can be the same or different.

“Individual” as used herein is a mammal, including humans. In some embodiments, individuals include pig, bovine, feline, canine, primate, rodent, or human. In some embodiments, the individual is human.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

The term “therapeutically effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, a therapeutically effective amount comprises an amount sufficient to cause a tumor to shrink and/or to decrease the growth rate of the tumor (such as to suppress tumor growth) or to prevent or delay other unwanted cell proliferation. In some embodiments, a therapeutically effective amount is an amount sufficient to delay development. In some embodiments, a therapeutically effective amount is an amount sufficient to prevent or delay occurrence and/or recurrence. A therapeutically effective amount can be administered in one or more administrations.

The term “carrier,” as used herein, refers to relatively nontoxic chemical compounds or agents that facilitate the incorporation of a compound into cells or tissues.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound, which are not biologically or otherwise undesirable, and which can be administered as drugs or pharmaceuticals to an individual. Pharmaceutically acceptable salts may be pharmaceutically acceptable acid addition salts. Examples of pharmaceutically acceptable acid addition salts include, but are not limited to, sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates. Pharmaceutically acceptable salts may be pharmaceutically acceptable base addition salts. Pharmaceutically acceptable base addition salts derived from inorganic bases include, but are not limited to, sodium, potassium, lithium, ammonium, calcium, magnesium, iron, zinc, copper, manganese, aluminum salts and the like. Pharmaceutically acceptable base addition salts derived from organic bases include, but are not limited to, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, and N-ethylpiperidine.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Compounds

In one aspect, provided herein is a compound of formula (I):

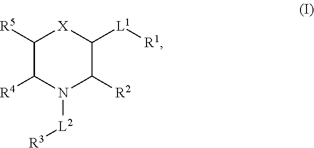

or a stereoisomer, tautomer, or a pharmaceutically acceptable salt of any of the foregoing, wherein:
X is —O— or —CHR⁶—,
   provided that when X is —O—, then L¹ is *—C(O)NH—** and L² is —C(O)—;
R¹ is C₃₋₁₂ cycloalkyl, 3- to 12-membered heterocyclyl, 5- to 12-membered heteroaryl, or C₆₋₁₄ aryl, each of which is independently optionally substituted with one or more R¹¹,
   wherein each R¹¹ is independently oxo, C₁₋₆ alkyl, C₂₋₆ alkenyl, C₂₋₆ alkynyl, halogen, —CN, —OR^{1a}, —SR^{1a}, —NR^{1a}R^{1b}, —NO₂, —C(O)R^{1a}, —OC(O)R^{1a}, —C(O)OR^{1a}, —C(O)NR^{1a}R^{1b}, —OC(O)NR^{1a}R^{1b}, —NR^{1a}C(O)R^{1b}, —NR^{1a}C(O)OR^{1b}, —S(O)R^{1a}, —S(O)₂R^{1a}, —NR^{1a}S(O)R^{1b}, —C(O )NR^{1a}S(O)R^{1b}, —NR^{1a}S(O)₂R^{1b}, —C(O)NR^{1a}S(O)₂R^{1b}, —S(O)NR^{1a}R^{1b}, —S(O)₂NR^{1a}R^{1b}, —P(O)R^{1a}R^{1b} C₃₋₆ cycloalkyl, 3- to 12-membered heterocyclyl, 5- to 12-membered heteroaryl, C₆₋₁₄ aryl, —(C₁₋₆ alkylene) NR^{1a}R^{1b}, —(C₁₋₆ alkylene) C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN,
      wherein R¹ and R^{1b} are each independently H, C₁₋₆ alkyl, C₂₋₆ alkenyl, C₂₋₆ alkynyl, C₃₋₁₂ cycloalkyl, 3- to 12-membered heterocyclyl, 5- to 12-membered heteroaryl, or C₆₋₁₄ aryl, or
         R^{1a} and R^{1b} are taken together with the nitrogen atom to which they attach to form a 3- to 12-membered heterocyclyl, which is optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN;
R² is C₃₋₁₂ cycloalkyl, 3- to 12-membered heterocyclyl, or C₆₋₁₄ aryl, each of which is independently optionally substituted with one or more -Q-W, wherein:
   Q is C₁₋₆ alkylene, —(N-L³-R^{Q})— or —O—,
      wherein R^{Q} is H, C₁₋₆ alkyl, 5- to 12-membered heteroaryl, or C₆₋₁₄ aryl, and L³ is —C(O)—, *—C(O)O—CH₂—**, or a bond, wherein * indicates the point of attachment to N and ** indicates the point of attachment to R^{Q},
   W is H, C₃₋₁₂ cycloalkyl, 3- to 12-membered heterocyclyl, 5- to 12-membered heteroaryl, or C₆₋₁₄ aryl, each of which is independently optionally substituted with one or more R⁷;
R³ is H, C₃₋₁₂ cycloalkyl, 3- to 12-membered heterocyclyl, 5- to 12-membered heteroaryl, or C₆₋₁₄ aryl, wherein the C₃₋₁₂ cycloalkyl, 3- to 12-membered heterocyclyl, 5- to 12-membered heteroaryl, and C₆₋₁₄ aryl are each independently optionally substituted with one or more R³¹,
   wherein each R³¹ is independently oxo, C₁₋₆ alkyl, C₂₋₆ alkenyl, C₂₋₆ alkynyl, halogen, —CN, —OR^{3a}, —SR^{3a}, —NR^{3a}R^{3b}, —NO₂, —C(O)R^{3a}, —OC(O)R^{3a}, —C(O)OR^{3a}, —C(O)NR^{3a}R^{3b}, —OC(O)NR^{3a}R^{3b}, —NR^{3a}C(O)R^{3b}, —NR^{3a}C(O)OR^{3b}, —S(O)R^{3a}, —S(O)₂R^{3a}, —NR^{3a}S(O)R^{3b}, —C(O )NR^{3a}S(O)R^{3b}, —NR^{3a}S(O)₂R^{3b}, —C(O)NR^{3a}S(O)₂R^{3b}, —S(O)NR^{3a}R^{3b}, —S(O)₂NR^{3a}R^{3b}, —P(O)R^{3a}R^{3b} C₃₋₆ cycloalkyl, 3- to 12-membered heterocyclyl, 5- to 12-membered heteroaryl, C₆₋₁₄ aryl, —(C₁₋₆ alkylene) NR^{3a}R^{3b}, —(C₁₋₆ alkylene)C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN,
      wherein R^{3a} and R^{3b} are each independently H, C₁₋₆ alkyl, C₂₋₆ alkenyl, C₂₋₆ alkynyl, C₃₋₁₂ cycloalkyl, 3- to 12-membered heterocyclyl, 5- to 12-membered heteroaryl, or C₆₋₁₄ aryl, or
         R^{3a} and R^{3b} are taken together with the nitrogen atom to which they attach to form a 3- to 12-membered heterocyclyl, which is optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN;
and
R⁴, R⁵, and R⁶ are each independently H, C₁₋₆ alkyl, C₂₋₆ alkenyl, C₂₋₆ alkynyl, halogen, —CN, hydroxyl, C₁₋₆ alkoxy, C₃₋₆ cycloalkyl, 3- to 12-membered heterocyclyl, 5- to 12-membered heteroaryl, or C₆₋₁₄ aryl, wherein the C₁₋₆ alkyl, C₂₋₆ alkenyl, C₂₋₆ alkynyl, C₁₋₆ alkoxy, C₃₋₆ cycloalkyl, 3- to 12-membered heterocyclyl, 5- to 12-membered heteroaryl, and C₆₋₁₄ aryl are each independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN, and wherein,
   R⁴ and R⁵ or R and R⁶ may be taken together with the carbon atoms to which they are attached to form a ring B which is independently optionally substituted with one or more R⁸, wherein ring B is C₃₋₁₂ cycloalkyl or 3- to 12-membered heterocyclyl, and
   R⁴ may be taken with the carbon atom to which it is attached, the nitrogen atom adjacent to the carbon atom, L², and part of R³ to form a 6- to 8-membered heterocyclyl;
each R⁷ is independently oxo, C₁₋₆ alkyl, C₂₋₆ alkenyl, C₂₋₆ alkynyl, halogen, —CN, —OR^{7a}, —SR^{7a}, —NR^{7a}R^{7b}, —NO₂, —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)R^{7a}R^{7b}, C₃₋₆ cycloalkyl, 3- to 12-membered heterocyclyl, 5- to 12-membered heteroaryl, C₆₋₁₄ aryl, —(C₁₋₆ alkylene) NR^{7a}R^{7b}, —(C₁₋₆ alkylene)C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN, wherein
   R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 12-membered heteroaryl, or C₆₋₁₄ aryl, or
      R^{7a} and R^{7b} are taken together with the nitrogen atom to which they attach to form a 3- to 12-membered heterocyclyl, which is optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN;
each R⁸ is independently oxo, C₁₋₆ alkyl, C₂₋₆ alkenyl, C₂₋₆ alkynyl, halogen, —CN, —OR^{8a}, —SR^{8a}, —NR^{8a}R^{8b}, —NO₂, —C(O)R^{8a}, —OC(O)R^{8a}, —C(O)OR^{8a}, —C(O)NR^{8a}R^{8b}, —OC(O)NR^{8a}R^{8b}, —NR^{8a}C(O)R^{8b}, —NR^{8a}C(O)OR^{8b}, —S(O)R^{8a}, —S(O)₂R^{8a}, —NR^{8a}S(O)R^{8b}, —C(O )NR^{8a}S(O)R^{8b}, —NR^{8a}S(O)₂R^{8b}, —C(O)NR^{8a}S(O)₂R^{8b}, —S(O)NR^{8a}R^{8b}, —S(O)₂NR^{8a}R^{8b}, —P(O)R^{8a}R^{8b}, C₃₋₆ cycloalkyl, 3- to 12-membered heterocyclyl, 5- to 12-membered heteroaryl, C₆₋₁₄ aryl, —(C₁₋₆ alkylene) NR^{8a}R^{8b}, —(C₁₋₆ alkylene)C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN, wherein
   R^{8a} and R^{8b} are each independently H, C₁₋₆ alkyl, C₂₋₆ alkenyl, C₂₋₆ alkynyl, C₃₋₁₂ cycloalkyl, 3- to 12-membered heterocyclyl, 5- to 12-membered heteroaryl, or C₆₋₁₄ aryl, or
      R^{8a} and R^{8b} are taken together with the nitrogen atom to which they attach to form a 3- to 12-membered heterocyclyl, which is optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN;
L¹ is *—C(O)NH—**, a bond, —C(O)—, *—CH₂—NH—**, or *—C(O)NH—CH₂—**, wherein * indicates the point of attachment to the carbon atom of the piperidine and ** indicates the point of attachment to R¹;
L² is —C(O)—, a bond, —CH₂—, —S(O)₂—, or #—S(O)₂—CH₂— ##, wherein #indicates the point of attachment to the nitrogen atom and ##indicates the point of attachment to R³,
   provided that when X is —CHR⁶— and R⁴, R⁵, and R⁶ are all H, then at least one of the following conditions apply:
      (1) L¹ is a bond, —C(O)—, *—CH₂—NH—**, or *—C(O)NH—CH₂—**
      (2) L² is a bond, —CH₂—, —S(O)₂—, or #—S(O)₂—CH₂— ##, and
      (3) R² is phenyl substituted with one or more -Q-W, wherein Q is —(N-L³-R^{Q})— and R^{Q} is 5- to 12-membered heteroaryl or C₆₋₁₄ aryl.

In some embodiments of a compound of formula (I), or a stereoisomer, tautomer, or a pharmaceutically acceptable salt of any of the foregoing, the compound is of formula (I-a) or formula (I-b), wherein R¹, R², R³, R⁴, R⁵, X, L¹, and L² are detailed herein for formula (I). In some embodiments, the compound is of formula (I-a). In some embodiments, the compound is of formula (I-b).

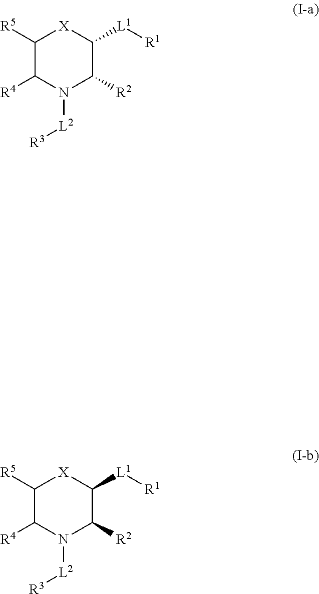

Specific values described herein are values for a compound of formula (I) or any variation thereof where applicable, such as any one of formulae (I-a), (I-b), (II), (II-a)-(II-b), (III), (III-a), (III-b), (IV), (IV-a), (IV-b), (V), (V-a)-(V-k), (VI), (VI-a), (VI-b), (VII), (VII-a) and (VII-b). It is to be understood that two or more values may combined. Thus, it is to be understood that any variable for a compound of formula (I) or any variation thereof may be combined with any other variable for a compound of formula (I) or any variation thereof the same as if each and every combination of variables were specifically and individually listed.

In some embodiments of a compound of formula (I) or any variation thereof where applicable, or a stereoisomer, tautomer, or a pharmaceutically acceptable salt of any of the foregoing, X is —O—. In some embodiments, X is —CHR⁶—.

In some embodiments of a compound of formula (I) or any variation thereof where applicable, or a stereoisomer, tautomer, or a pharmaceutically acceptable salt of any of the foregoing, L¹ is *—C(O)NH—**. In some embodiments, L¹ is a bond. In some embodiments, L¹ is —C(O)—. In some embodiments, L¹ is *—CH₂—NH—**. In some embodiments, L¹ is *—C(O)NH—**, a bond, —C(O)—, or *—C(O)NH—CH₂—**. In some embodiments, L¹ is a bond, —C(O)—, or *—C(O)NH—CH₂—**. In some embodiments, L¹ is *—C(O)NH—**, a bond, —C(O)—, or *—C(O)NH—CH₂—**.

In some embodiments of a compound of formula (I) or any variation thereof where applicable, or a stereoisomer, tautomer, or a pharmaceutically acceptable salt of any of the foregoing, L² is a —C(O)—. In some embodiments, L² is a bond. In some embodiments, L² is —S(O)₂—. In some embodiments, L² is —CH₂—. In some embodiments, L² is #—S(O)₂—CH₂— ##. In some embodiments, L² is —C(O)—, a bond, —S(O)₂—, or #—S(O)₂—CH₂— ##. In some embodiments, L² is a bond, —S(O)₂—, —CH₂—, or *—S(O)₂—CH₂—**. In some embodiments, L² is a bond, —S(O)₂—, or #—S(O)₂—CH₂— ##.

In some embodiments of a compound of formula (I) or any variation thereof where applicable, or a stereoisomer, tautomer, or a pharmaceutically acceptable salt of any of the foregoing, L¹ is *—C(O)NH—**, a bond, or *—CH₂—NH—**; and L² is —C(O)—, a bond, —S(O)₂—, —CH₂—, or #—S(O)₂—CH₂— ##. In some embodiments, L¹ is *—C(O)NH—**, a bond, —C(O)—, or *—C(O)NH—CH₂—**; and L² is —C(O)—, a bond, —S(O)₂—, or #—S(O)₂—CH₂— ##. In some embodiments, L¹ is *—C(O)NH—** and L² is a bond. In some embodiments, L¹ is *—C(O)NH—** and L² is —S(O)₂—. In some embodiments, L¹ is *—C(O)NH—** and L² is #—S(O)₂—CH₂— ##. In some embodiments, L¹ is *—C(O)NH—** and L² is —C(O)—. In some embodiments, L¹ is a bond and L² is —C(O)—. In some embodiments, L¹ is *—CH₂—NH—** and L² is —C(O)—. In some embodiments, L¹ is —C(O)— and L² is —C(O)—.

In some embodiments of a compound of formula (I) or any variation thereof where applicable, or a stereoisomer, tautomer, or a pharmaceutically acceptable salt of any of the foregoing, R⁴ is H. In some embodiments, R⁴ is C₁₋₆ alkyl. In some embodiments, R⁴ is C₂₋₆ alkenyl. In some embodiments, R⁴ is halogen. In some embodiments, R⁴ is —CN. In some embodiments, R⁴ is hydroxyl. In some embodiments, R⁴ is C₁₋₆ alkoxy. In some embodiments, R⁴ is C₃₋₆ cycloalkyl. In some embodiments, R⁴ is 3- to 12-membered heterocyclyl. In some embodiments, R⁴ is 5- to 12-membered heteroaryl. In some embodiments, R⁴ is C₆₋₁₄ aryl.

In some embodiments of a compound of formula (I) or any variation thereof where applicable, or a stereoisomer, tautomer, or a pharmaceutically acceptable salt of any of the foregoing, R is H. In some embodiments, R⁵ is C₁₋₆ alkyl. In some embodiments, R is C₂₋₆ alkenyl. In some embodiments, R⁵ is halogen. In some embodiments, R⁵ is —CN. In some embodiments, R⁵ is hydroxyl. In some embodiments, R⁵ is C₁₋₆ alkoxy. In some embodiments, R⁵ is C₃₋₆ cycloalkyl. In some embodiments, R⁵ is 3- to 12-membered heterocyclyl. In some embodiments, R⁵ is 5- to 12-membered heteroaryl. In some embodiments, R⁵ is C₆₋₁₄ aryl. In some embodiments, R⁵ is H or hydroxyl.

In some embodiments of a compound of formula (I) or any variation thereof where applicable, or a stereoisomer, tautomer, or a pharmaceutically acceptable salt of any of the foregoing, R⁶ is H. In some embodiments, R⁶ is C₁₋₆ alkyl. In some embodiments, R⁶ is C₂₋₆ alkenyl. In some embodiments, R⁶ is halogen. In some embodiments, R⁶ is —CN. In some embodiments, R⁶ is hydroxyl. In some embodiments, R⁶ is C₁₋₆ alkoxy. In some embodiments, R⁶ is C₃₋₆ cycloalkyl. In some embodiments, R⁶ is 3- to 12-membered heterocyclyl. In some embodiments, R⁶ is 5- to 12-membered heteroaryl. In some embodiments, R⁶ is C₆₋₁₄ aryl. In some embodiments, X is —CHR⁶—; and R⁶ is H.

In some embodiments of a compound of formula (I) or any variation thereof where applicable, or a stereoisomer, tautomer, or a pharmaceutically acceptable salt of any of the foregoing, R⁴ is H; R⁵ is H or hydroxyl; X is —CHR⁶—; and R⁶ is H. In some embodiments, R⁴ is H; R is H; X is —CHR⁶—; and R⁶ is H. In some embodiments, R⁴ is H; R⁵ is H; X is —CHR⁶—; R⁶ is H; and L¹ is a bond, —C(O)—, *—CH₂—NH—**, or *—C(O)NH—CH₂—**. In some embodiments, R⁴ is H; R⁵ is H; X is —CHR⁶—; R⁶ is H; and L¹ is a bond, —C(O)—, or *—C(O)NH—CH₂—**. In some embodiments, R⁴ is H; R⁵ is H; X is —CHR⁶—; R⁶ is H; and L² is a bond, —CH₂—, —S(O)₂—, or #—S(O)₂—CH₂— ##. In some embodiments, R⁴ is H; R⁵ is H; X is —CHR⁶—; R⁶ is H; and L² is a bond, —S(O)₂—, or #—S(O)₂—CH₂— ##. In some embodiments, R⁴ is H; R⁵ is H; X is —CHR⁶—; R⁶ is H; and R² is phenyl substituted with one or more -Q-W, wherein Q is —(N-L³-R^{Q})— and R^{Q} is 5- to 12-membered heteroaryl or C₆₋₁₄ aryl.

In some embodiments of a compound of formula (I), or a stereoisomer, tautomer, or a pharmaceutically acceptable salt of any of the foregoing, the compound is of formula (II), (II-a), or (II-b), wherein R¹, R², R³, R⁴, R⁵, and R⁶ are detailed herein for formula (I); and L² is a bond, —CH₂—, —S(O)₂—, or #—S(O)₂—CH₂— ##. In some embodiments, L² is a bond, —S(O)₂—, or #—S(O)₂—CH₂— ##. In some embodiments, L² is a bond. In some embodiments, L² is —CH₂—. In some embodiments, L² is —S(O)₂—. In some embodiments, L² is #—S(O)₂—CH₂— ##. In some embodiments, the compound is of formula (II). In some embodiments, the compound is of formula (II-a). In some embodiments, the compound is of formula (II-b).

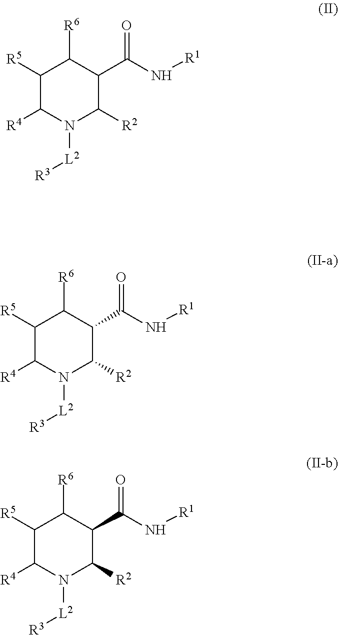

In some embodiments of a compound of formula (I), or a stereoisomer, tautomer, or a pharmaceutically acceptable salt of any of the foregoing, the compound is of formula (III), (III-a), or (III-b), wherein R¹, R², R³, R⁴, R⁵, and R⁶ are detailed herein for formula (I); and L¹ is a bond, —C(O)—, *—CH₂—NH—**, or *—C(O)NH—CH₂—**. In some embodiments, L¹ is a bond, —C(O)—, or *—C(O)NH—CH₂—**. In some embodiments, L¹ is a bond. In some embodiments, L¹ is —C(O)—. In some embodiments, L¹ is *—CH₂—NH—**. In some embodiments, L¹ is *—C(O)NH—CH₂—**. In some embodiments, the compound is of formula (III). In some embodiments, the compound is of formula (III-a). In some embodiments, the compound is of formula (III-b).

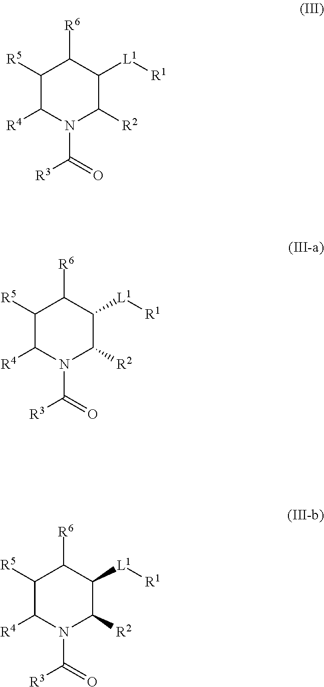

In some embodiments of a compound of formula (I), or a stereoisomer, tautomer, or a pharmaceutically acceptable salt of any of the foregoing, the compound is of formula (IV), (IV-a), or (IV-b), wherein R¹, R³, R⁴, R⁵, R⁶, W, L¹, and L² are detailed herein for formula (I); and R^{Q} is 5- to 12-membered heteroaryl or C₆₋₁₄ aryl. In some embodiments, the compound is of formula (IV). In some embodiments, the compound is of formula (IV-a). In some embodiments, the compound is of formula (IV-b).

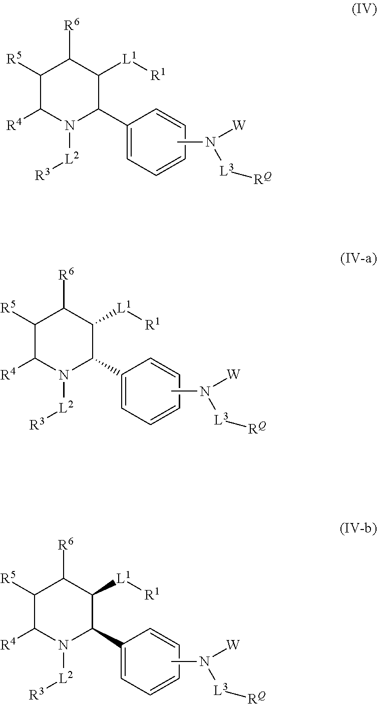

In some embodiments of a compound of formula (I) or any variation thereof where applicable, or a stereoisomer, tautomer, or a pharmaceutically acceptable salt of any of the foregoing, R⁴ and R⁵ are taken together with the carbon atoms to which they are attached to form a ring B which is optionally substituted with one or more R⁸, wherein ring B is C₃₋₁₂ cycloalkyl, 3- to 12-membered heterocyclyl, 5- to 12-membered heteroaryl, or C₆₋₁₄ aryl. In some embodiments, R and R⁶ are taken together with the carbon atoms to which they are attached to form a ring B which is optionally substituted with one or more R^{B}, wherein ring B is C₃₋₁₂ cycloalkyl, 3- to 12-membered heterocyclyl, 5- to 12-membered heteroaryl, or C₆₋₁₄ aryl.

In some embodiments of a compound of formula (I), or a stereoisomer, tautomer, or a pharmaceutically acceptable salt of any of the foregoing, the compound is of formula (V), (V-a), (V-b), (VI), (VI-a), or (VI-b), wherein R¹, R², R³, R⁴, R⁶, L¹, and L² are detailed herein for formula (I). In some embodiments, L¹ is *—C(O)NH—** and L² is —C(O)—. In some embodiments, the compound is of formula (V). In some embodiments, the compound is of formula (V-a). In some embodiments, the compound is of formula (V-b). In some embodiments, the compound is of formula (VI). In some embodiments, the compound is of formula (VI-a). In some embodiments, the compound is of formula (VI-b).

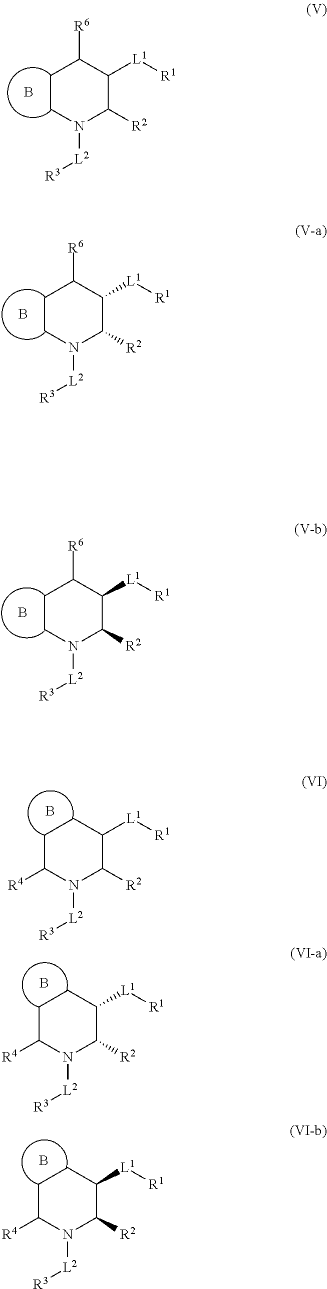

In some embodiments of a compound of formula (I) or any variation thereof where applicable, or a stereoisomer, tautomer, or a pharmaceutically acceptable salt of any of the foregoing, ring B is a C₃₋₁₂ cycloalkyl, which is optionally substituted with one or more R. In some embodiments, ring B is C₃₋₆ cycloalkyl, which is optionally substituted with one or more R⁸. In some embodiments, ring B is cyclopropyl, cyclobutyl, cyclopentyl, or cyclohexyl, each of which is independently optionally substituted with one or more R⁸. In some embodiments, ring B is cyclopentyl, which is optionally substituted with one or more R⁸. In some embodiments, ring B is a 3- to 12-membered heterocyclyl, which is optionally substituted with one or more R⁸. In some embodiments, ring B is a 3- to 6-membered heterocyclyl, which is optionally substituted with one or more R⁸. In some embodiments, ring B is tetrahydrofuranyl, pyrrolidinyl, piperidinyl, piperazinyl, morpholinyl, or thiomorpholinyl, each of which is independently optionally substituted with one or more R⁸. In some embodiments, ring B is tetrahydrofuranyl or pyrrolidinyl, each of which is independently optionally substituted with one or more R. In some embodiments, ring B is ring B is a C₃₋₁₂ cycloalkyl or 3- to 12-membered heterocyclyl, each of which is optionally substituted with one or more R⁸. In some embodiments, ring B is cyclopentyl, tetrahydrofuranyl, or pyrrolidinyl, each of which is independently optionally substituted with one or more R⁸. In some embodiments, ring B is tetrahydrofuranyl, which is optionally substituted with one or more R⁸. In some embodiments, ring B is pyrrolidinyl, which is optionally substituted with one or more R⁸. In some embodiments, ring B is

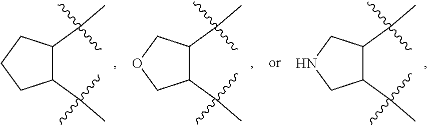 [SMILES: *C1CCCC1* ; *C1COCC1* ; *C1CNCC1*]

each of which is independently optionally substituted with one or more R⁸.

In some embodiments of a compound of formula (I), or a stereoisomer, tautomer, or a pharmaceutically acceptable salt of any of the foregoing, the compound is of formula (V-c), (V-d), (V-e), (V-f), (V-g), (V-h), (V-i), (V-j), or (V-k), wherein R¹, R², R³, R⁶, R⁸, L¹, and L² are detailed herein for formula (I) and n is 0, 1, 2, or 3. In some embodiments, the compound is of formula (V-c). In some embodiments, the compound is of formula (V-d). In some embodiments, the compound is of formula (V-e). In some embodiments, the compound is of formula (V-f). In some embodiments, the compound is of formula (V-g). In some embodiments, the compound is of formula (V-h). In some embodiments, the compound is of formula (V-i). In some embodiments, the compound is of formula (V-j). In some embodiments, the compound is of formula (V-k).

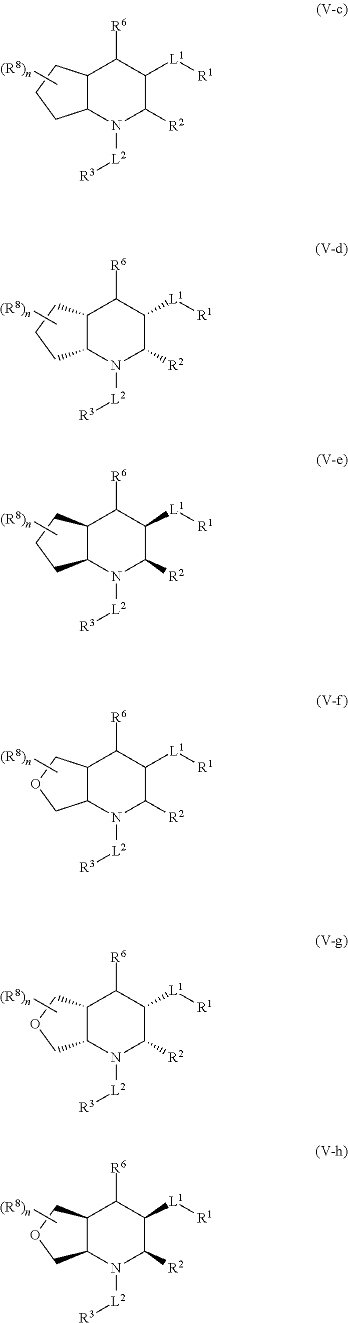

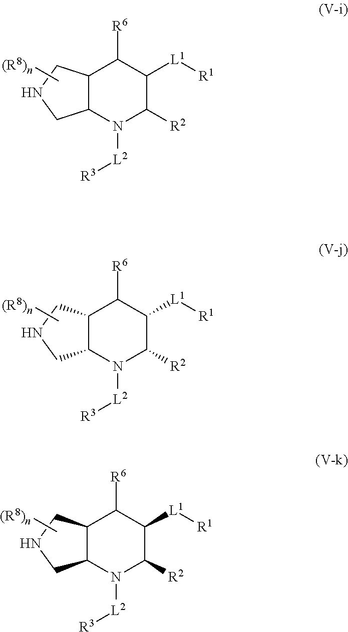

In some embodiments of a compound of formula (I) or any variation thereof where applicable, or a stereoisomer, tautomer, or a pharmaceutically acceptable salt of any of the foregoing, each R⁸ is independently C₁₋₆ alkyl, C₃₋₆ cycloalkyl, 3- to 12-membered heterocyclyl, or —C(O)Ra, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN. In some embodiments, each R⁸ is independently C₁₋₆ alkyl, C₃₋₆ cycloalkyl, 3- to 12-membered heterocyclyl, or —C(O)R^{B}a, each of which is independently optionally substituted with one or more halogen. In some embodiments, ring B is

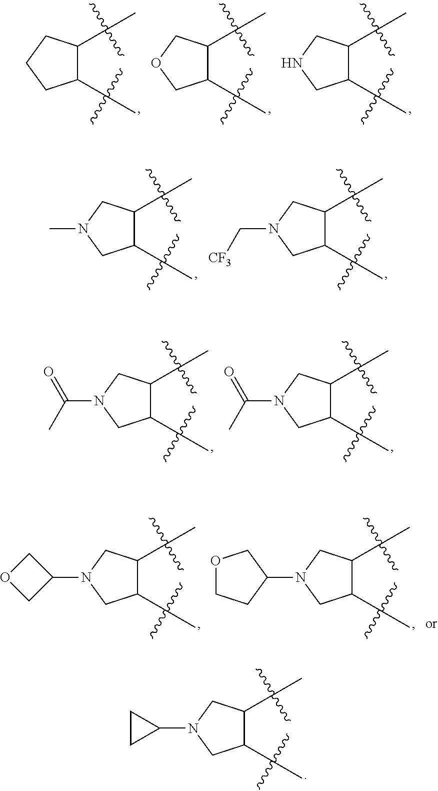

In some embodiments of a compound of formula (I), or a stereoisomer, tautomer, or a pharmaceutically acceptable salt of any of the foregoing, the compound is of formula (VII), (VII-a), or (VII-b), wherein R¹, R², R³, R⁴, and R are detailed herein for formula (I). In some embodiments, the compound is of formula (VII). In some embodiments, the compound is of formula (VII-a). In some embodiments, the compound is of formula (VII-b).

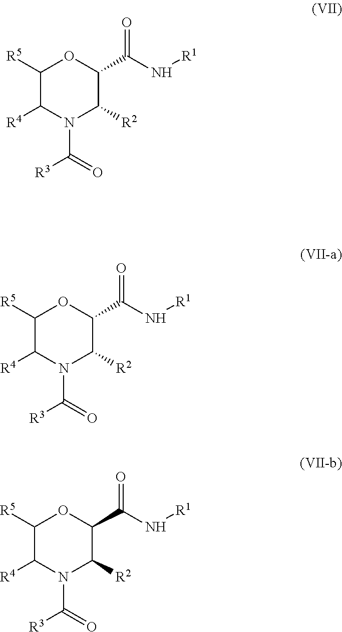

In some embodiments of a compound of formula (I) or any variation thereof where applicable, or a stereoisomer, tautomer, or a pharmaceutically acceptable salt of any of the foregoing, R⁴ is taken with the carbon atom to which it is attached, the nitrogen atom adjacent to the carbon atom, L², and part of R³ to form a 6- to 8-membered heterocyclyl. In some embodiments, R⁴ is taken with the carbon atom to which it is attached, the nitrogen atom adjacent to the carbon atom, L², and part of R³ to form a 6-membered heterocyclyl. In some embodiments, R⁴ is taken with the carbon atom to which it is attached, the nitrogen atom adjacent to the carbon atom, L², and part of R³ to form a 7-membered heterocyclyl. In some embodiments, R⁴ is taken with the carbon atom to which it is attached, the nitrogen atom adjacent to the carbon atom, L², and part of R³ to form an 8-membered heterocyclyl.

In some embodiments of a compound of formula (I) or any variation thereof where applicable, or a stereoisomer, tautomer, or a pharmaceutically acceptable salt of any of the foregoing, R¹ is C₃₋₁₂ cycloalkyl, which is optionally substituted with one or more R¹. In some embodiments, R¹ is 3- to 12-membered heterocyclyl, which is optionally substituted with one or more R¹¹. In some embodiments, R¹ is selected from the group consisting of

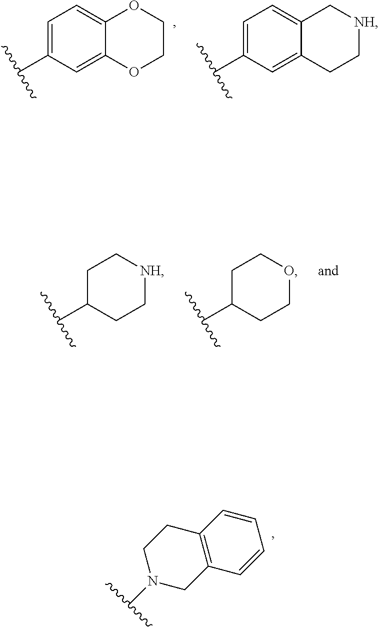 [SMILES: *c1ccc2c(c1)OCCO2 ; *c1ccc2c(c1)CCNC2 ; *C1CCNCC1 ; *C1CCOCC1 ; *N1CCc2ccccc2C1]

each of which is independently optionally substituted with one or more R¹¹. In some embodiments, R¹ is 5- to 12-membered heteroaryl, which is optionally substituted with one or more R¹¹. In some embodiments, R¹ is selected from the group consisting of

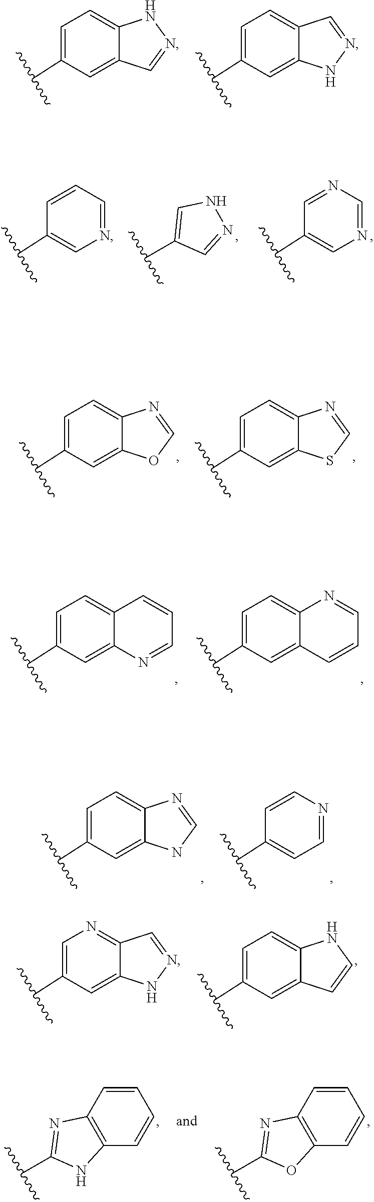

each of which is independently optionally substituted with one or more R¹¹. In some embodiments, R¹ is C₆₋₁₄ aryl, which is optionally substituted with one or more R¹¹. In some embodiments, R¹ is phenyl, which is optionally substituted with one or more R¹¹. In some embodiments, R¹ is —(C₁₋₆ alkylene) C₃₋₁₂ cycloalkyl, which is optionally substituted with one or more R¹¹. In some embodiments, R¹ is —(C₁₋₆ alkylene) 3- to 12-membered heterocyclyl, which is optionally substituted with one or more R¹¹. In some embodiments, R¹ is —(C₁₋₆ alkylene) 5- to 12-membered heteroaryl, which is optionally substituted with one or more R¹¹. In some embodiments, R¹ is —(C₁₋₆ alkylene) C₆₋₁₄ aryl, which is optionally substituted with one or more R¹¹. In some embodiments, R¹ is 3- to 12-membered heterocyclyl, C₆₋₁₄ aryl, or 5- to 12-membered heteroaryl, each of which is optionally substituted with one or more R¹¹. In some embodiments, R¹ is selected from the group consisting of

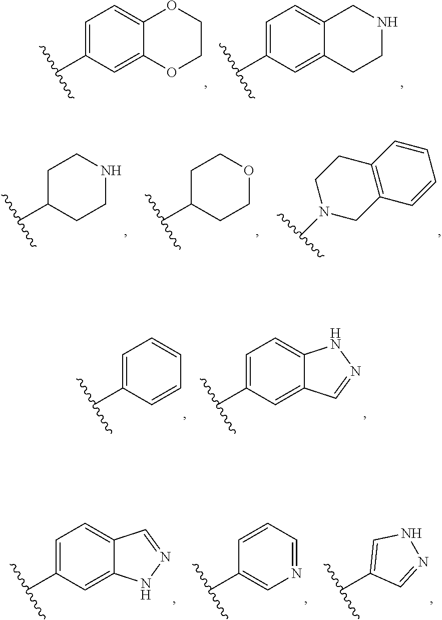

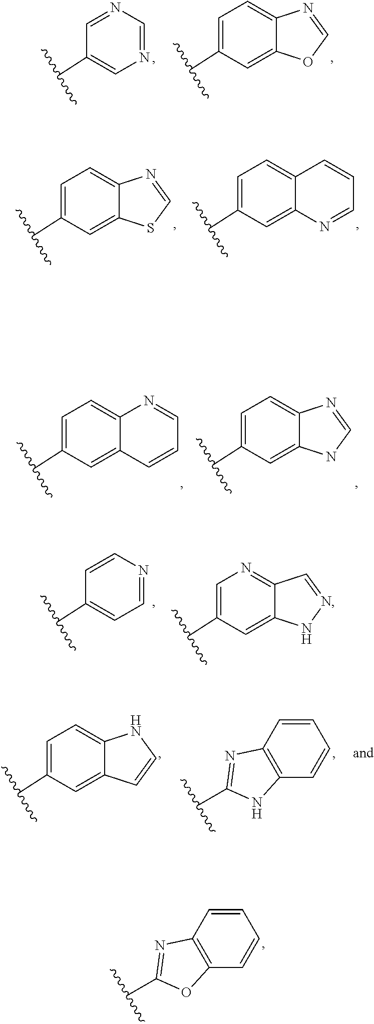

each of which is optionally substituted with one or more R¹¹.

In some embodiments of a compound of formula (I) or any variation thereof where applicable, or a stereoisomer, tautomer, or a pharmaceutically acceptable salt of any of the foregoing, each R¹¹ is independently C₁₋₆ alkyl, —NR^{1a}R^{1b}, halogen, —CN, —OR^{1a}, —NR^{1a}C(O)R^{1b}, —S(O)₂R^{1a}, —P(O)R^{1a}R^{1b}, 3- to 12-membered heterocyclyl, 5- to 12-membered heteroaryl, —(C₁₋₆ alkylene) 5- to 12-membered heteroaryl, —(C₁₋₆ alkylene) NR^{1a}R^{1b}, or —(C₁₋₆ alkylene) C₃₋₆ cycloalkyl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN. In some embodiments, each R¹¹ is independently C₁₋₆ alkyl, —NR^{1a}R^{1b}, halogen, —CN, —OR^{1a}, —NR^{1a}C(O)R^{1b}, —S(O)₂R^{1a}, or —P(O)R^{1a}R^{1b}, each of which is independently optionally substituted with one or more halogen. In some embodiments, each R¹¹ is independently C₁₋₆ alkyl, 3- to 12-membered heterocyclyl, halogen, 5- to 12-membered heteroaryl, —(C₁₋₆ alkylene) 5- to 12-membered heteroaryl, —(C₁₋₆ alkylene) NR^{1a}R^{1b}, —(C₁₋₆ alkylene)C₃₋₆ cycloalkyl, each of which is independently optionally substituted with one or more substituents selected from the group consisting of halogen, C₁₋₆ alkyl, and hydroxyl. In some embodiments, each R¹¹ is independently C₁₋₆ alkyl or halogen. In some embodiments, R¹ is 3- to 12-membered heterocyclyl

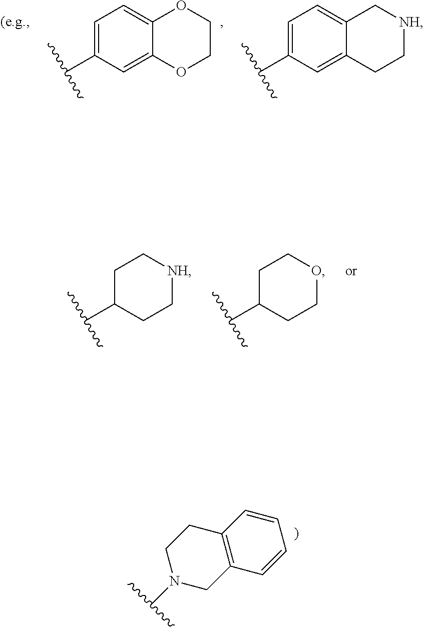 [SMILES: *c1ccc2c(c1)OCCO2 ; *c1ccc2c(c1)CCNC2 ; *C1CCNCC1 ; *C1CCOCC1 ; *N1CCc2ccccc2C1]

optionally substituted with one or more R¹¹, wherein each R¹¹ is independently C₁₋₆ alkyl or halogen. In some embodiments, R¹ is C₆₋₁₄ aryl (e.g., phenyl) optionally substituted with one or more R¹¹, wherein each R¹¹ is independently C₁₋₆ alkyl, —NR^{1a}R^{1b}, halogen, —CN, —OR^{1a}, —NR^{1a}C(O)R^{1b}, —S(O)₂R^{1a}, or —P(O)R^{1a}R^{1b}, each of which is independently optionally substituted with one or more halogen. In some embodiments, R¹ is 5- to 12-membered heteroaryl

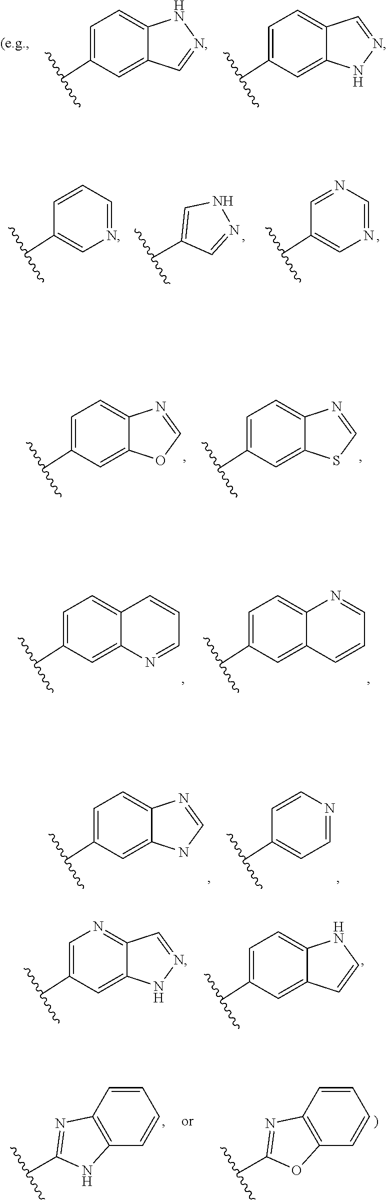

optionally substituted with one or more R¹¹, wherein each R¹¹ is independently C₁₋₆ alkyl, 3- to 12-membered heterocyclyl, halogen, 5- to 12-membered heteroaryl, —(C₁₋₆ alkylene) 5- to 12-membered heteroaryl, —(C₁₋₆ alkylene) NR^{1a}R^{1b}, —(C₁₋₆ alkylene)C₃₋₆ cycloalkyl, each of which is independently optionally substituted with one or more substituents selected from the group consisting of halogen, C₁₋₆ alkyl, and hydroxyl.

In some embodiments of a compound of formula (I) or any variation thereof where applicable, or a stereoisomer, tautomer, or a pharmaceutically acceptable salt of any of the foregoing, R¹ is selected from the group consisting of:

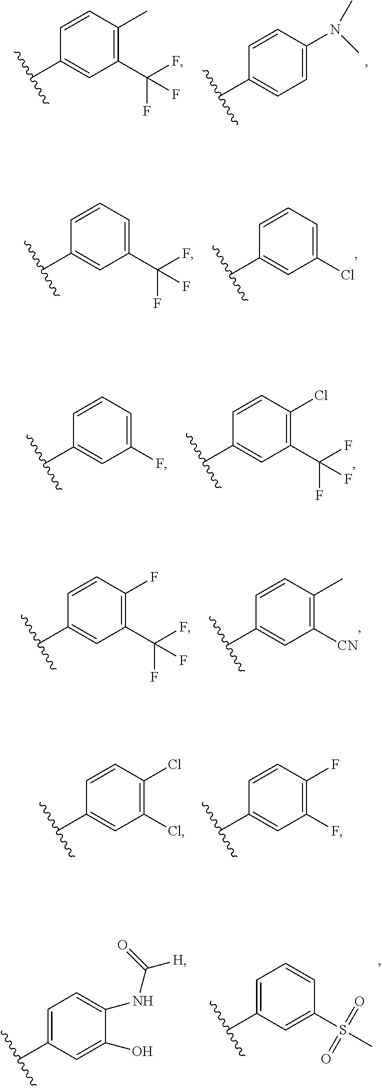

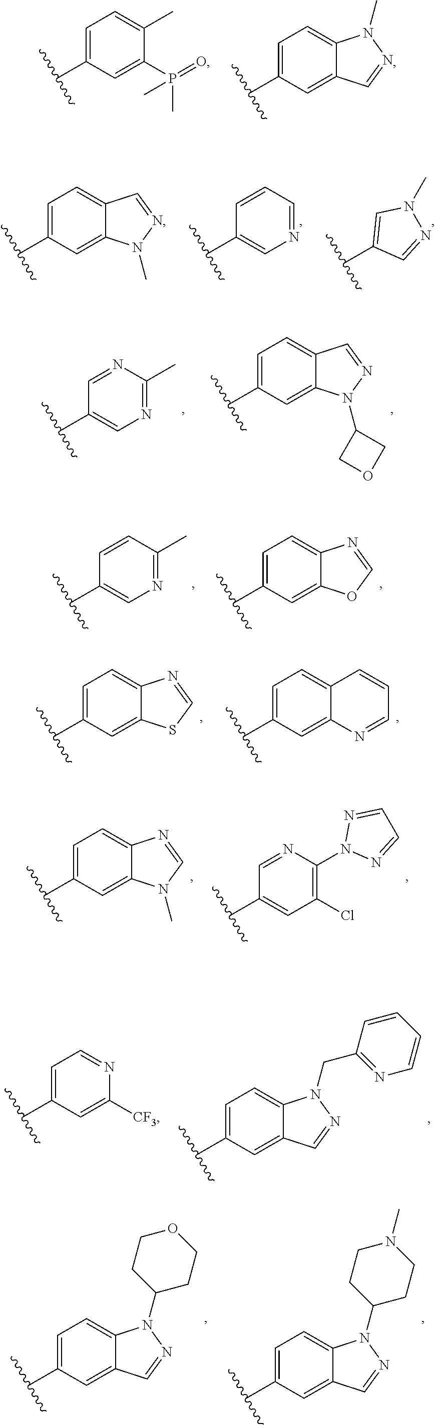

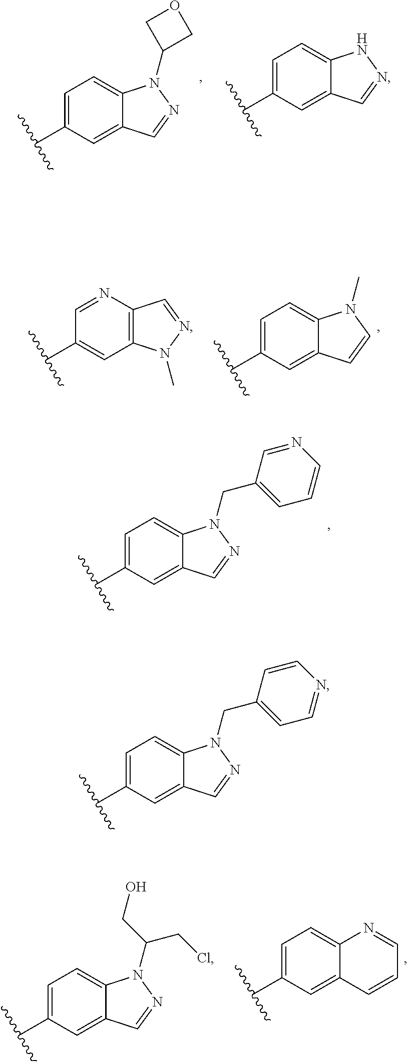

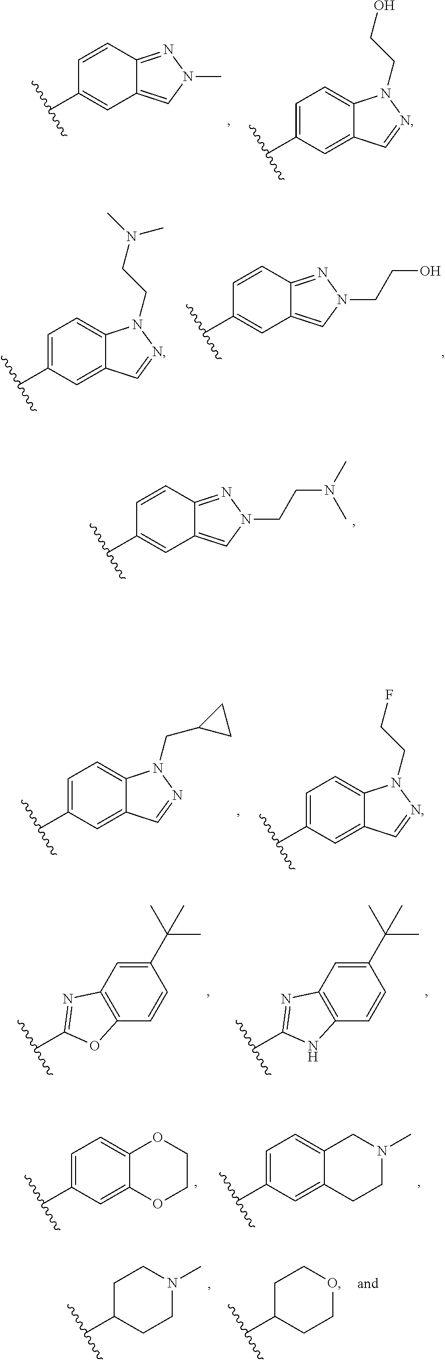

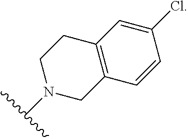 [SMILES: *N1CCc2cc(Cl)ccc2C1]

In some embodiments of a compound of formula (I) or any variation thereof where applicable, or a stereoisomer, tautomer, or a pharmaceutically acceptable salt of any of the foregoing, R² is C₃₋₁₂ cycloalkyl, which is independently optionally substituted with one or more -Q-W. In some embodiments, R² is 3- to 12-membered heterocyclyl, which is optionally substituted with one or more -Q-W. In some embodiments, R² is

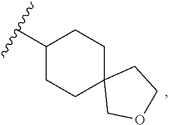 [SMILES: *C1CCC2(CCOC2)CC1]

which is optionally substituted with one or more -Q-W. In some embodiments, R² is C₆₋₁₄ aryl, which is optionally substituted with one or more -Q-W. In some embodiments, R² is phenyl, which is optionally substituted with one or more -Q-W. In some embodiments, R² is

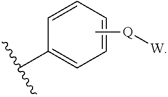

In some embodiments, R² is

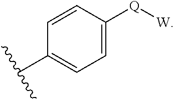

In some embodiments of a compound of formula (I) or any variation thereof where applicable, or a stereoisomer, tautomer, or a pharmaceutically acceptable salt of any of the foregoing, Q is C₁₋₆ alkylene. In some embodiments, Q is —CH₂—. In some embodiments, Q is —O—. In some embodiments, Q is —(N-L³-R^{Q})—. In some embodiments, Q is —NR^{Q}—, wherein R^{Q} is H or C₁₋₆ alkyl. In some embodiments, Q is —NR^{Q}—. In some embodiments, Q is —NR^{Q}—, wherein R^{Q} is H. In some embodiments, Q is —(N-L³-R^{Q})—, wherein R^{Q} is 5- to 12-membered heteroaryl or C₆₋₁₄ aryl.

In some embodiments of a compound of formula (I) or any variation thereof where applicable, or a stereoisomer, tautomer, or a pharmaceutically acceptable salt of any of the foregoing, W is C₃₋₁₂ cycloalkyl, which is independently optionally substituted with one or more R⁷. In some embodiments, W is C₃₋₆ cycloalkyl, which is optionally substituted with one or more R⁷. In some embodiments, W is

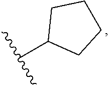 [SMILES: *C1CCCC1]

which is optionally substituted with one or more R⁷. In some embodiments, W is 3- to 12-membered heterocyclyl, which is optionally substituted with one or more R⁷. In some embodiments, W is selected from the group consisting of

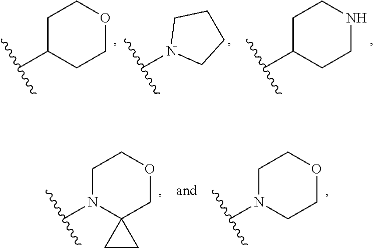

each of which is independently optionally substituted with one or more R⁷. In some embodiments, W is 5- to 12-membered heteroaryl, which is optionally substituted with one or more R⁷. In some embodiments, W is C₆₋₁₄ aryl, which is optionally substituted with one or more R⁷. In some embodiments, W is selected from the group consisting of

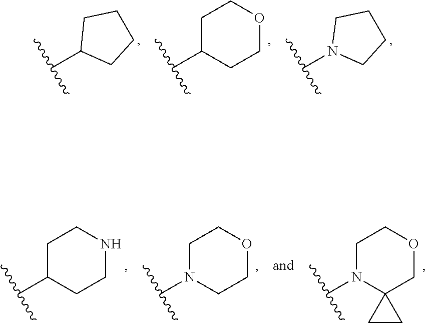 [SMILES: *C1CCCC1 ; *C1CCOCC1 ; *N1CCCC1 ; *C1CCNCC1 ; *N1CCOCC1 ; *N1CCOCC12CC2]

each of which is independently optionally substituted with one or more R⁷.

In some embodiments of a compound of formula (I) or any variation thereof where applicable, or a stereoisomer, tautomer, or a pharmaceutically acceptable salt of any of the foregoing, each R⁷ is independently oxo, C₁₋₆ alkyl, or halogen, wherein the C₁₋₆ alkyl is optionally substituted with one or more substituents selected from the group consisting of C₂₋₆ alkenyl, C₂₋₆ alkynyl, halogen, hydroxyl, C₁₋₆ alkoxy, and —CN. In some embodiments, each R⁷ is independently oxo, C₁₋₆ alkyl, or halogen, wherein the C₁₋₆ alkyl is optionally substituted with one or more halogen.

In some embodiments of a compound of formula (I) or any variation thereof where applicable, or a stereoisomer, tautomer, or a pharmaceutically acceptable salt of any of the foregoing, W is selected from the group consisting of

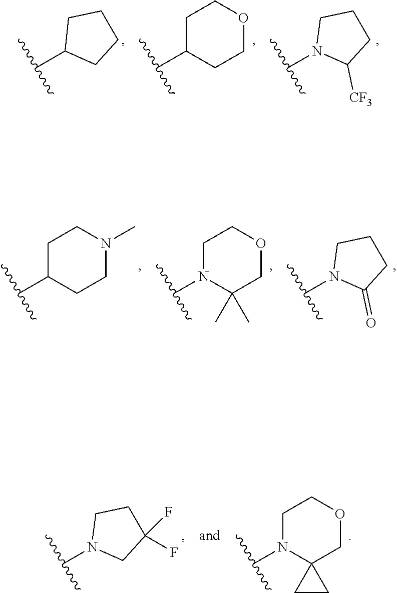

In some embodiments of a compound of formula (I) or any variation thereof where applicable, or a stereoisomer, tautomer, or a pharmaceutically acceptable salt of any of the foregoing, R³ is H. In some embodiments, R³ is 3- to 12-membered heterocyclyl, 5- to 12-membered heteroaryl, or C₆₋₁₄ aryl, each of which is independently optionally substituted with one or more R³¹. In some embodiments, R³ is C₆₋₁₄ aryl, which is optionally substituted with one or more R³¹. In some embodiments, R³ is phenyl, which is optionally substituted with one or more R³¹. In some embodiments, R³ is 3- to 12-membered heterocyclyl, which is optionally substituted with one or more R³¹. In some embodiments, R³ is

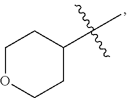 [SMILES: *C1CCOCC1]

which is optionally substituted with one or more R³¹. In some embodiments, 5- to 12-membered heteroaryl, which is optionally substituted with one or more R³¹. In some embodiments, R³ is selected from the group consisting of

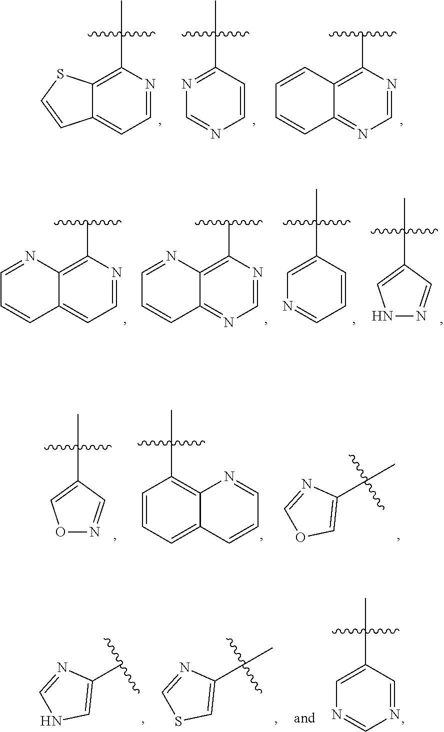

each of which is independently optionally substituted with one or more R³¹. In some embodiments R³ is selected from the group consisting of

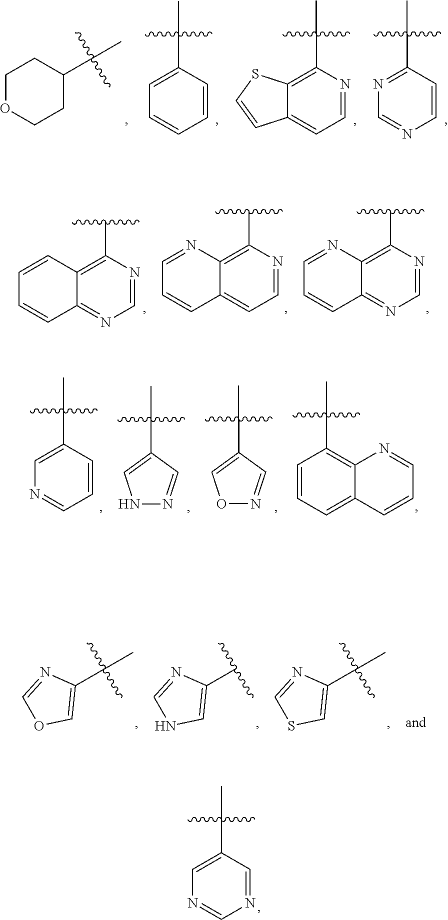

each of which is independently optionally substituted with one or more R³¹.

In some embodiments of a compound of formula (I) or any variation thereof where applicable, or a stereoisomer, tautomer, or a pharmaceutically acceptable salt of any of the foregoing, each R³¹ is independently C₁₋₆ alkyl, —CN, —NO₂, halogen, —OR^{3a}, —C(O)OR^{3a}, or —S(O)₂R^{3a}, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN. In some embodiments, each R³¹ is independently C₁₋₆ alkyl, —CN, —NO₂, halogen, —OR^{3a}, —C(O)OR^{3a}, or —S(O)₂R^{3a}, each of which is independently optionally substituted with one or more halogen. In some embodiments, each R³¹ is independently C₁₋₆ alkyl or halogen. In some embodiments, R³ is C₆₋₁₄ aryl (e.g., phenyl) optionally substituted with one or more R³¹, wherein each R³¹ is independently C₁₋₆ alkyl, —CN, —NO₂, halogen, —OR^{3a}, —C(O)OR^{3a}, or —S(O)₂R^{3a}, each of which is independently optionally substituted with one or more halogen. In some embodiments, R³ is 5- to 12-membered heteroaryl

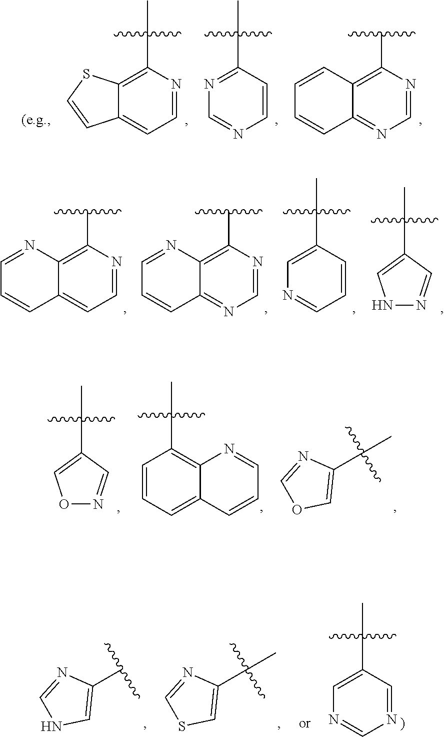

optionally substituted with one or more R³¹, wherein each R³ is independently C₁₋₆ alkyl or halogen.

In some embodiments of a compound of formula (I) or any variation thereof where applicable, or a stereoisomer, tautomer, or a pharmaceutically acceptable salt of any of the foregoing, R³ is selected from the group consisting of:

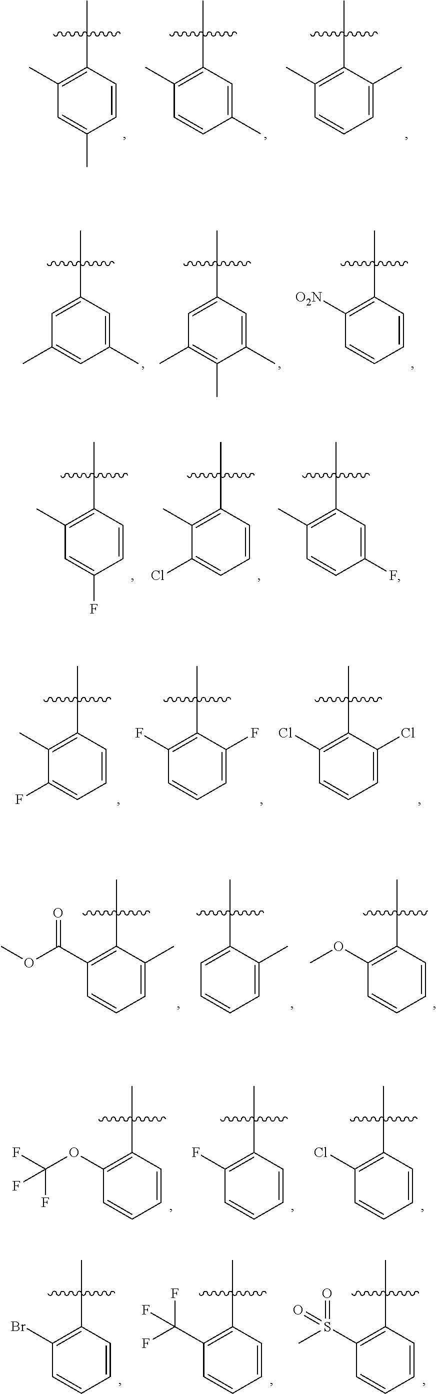

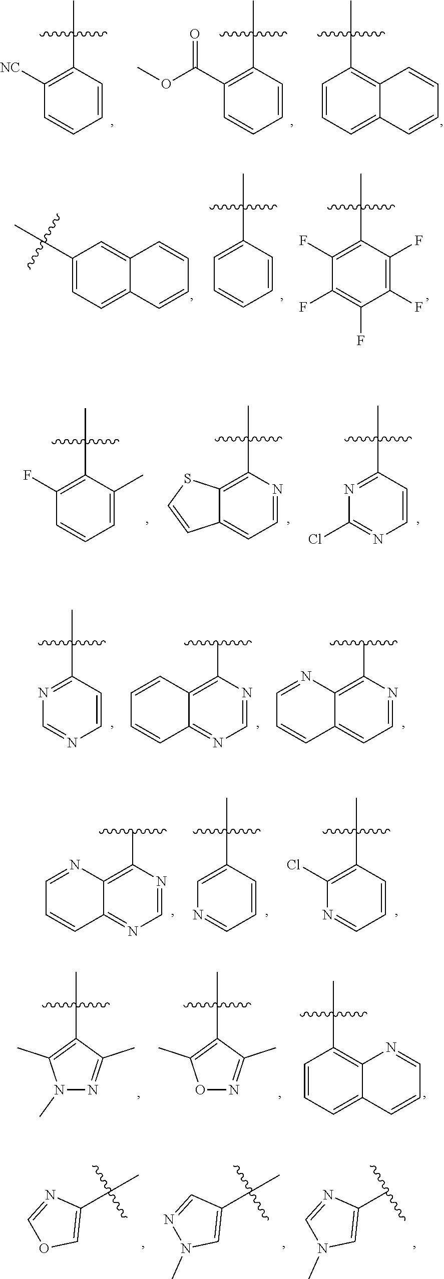

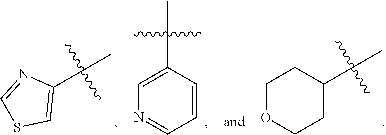 [SMILES: *c1cscn1 ; *c1cccnc1 ; *C1CCOCC1]

In some embodiments of a compound of formula (I), or a stereoisomer, tautomer, or a pharmaceutically acceptable salt of any of the foregoing, the compound is of formula (VIII),

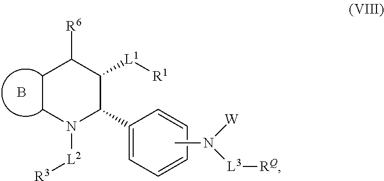

wherein R¹, R³, R⁶, B, W, L¹, L², L², and R^{Q} are detailed herein for formula (I). In some embodiments, ring B is a C₃₋₁₂ cycloalkyl, which is optionally substituted with one or more R. In some embodiments, ring B is C₃₋₆ cycloalkyl, which is optionally substituted with one or more R⁸. In some embodiments, ring B is cyclopropyl, cyclobutyl, cyclopentyl, or cyclohexyl, each of which is independently optionally substituted with one or more R. In some embodiments, ring B is cyclopentyl, which is optionally substituted with one or more R⁸. In some embodiments, ring B is a 3- to 12-membered heterocyclyl, which is optionally substituted with one or more R⁸. In some embodiments, ring B is a 3- to 6-membered heterocyclyl, which is optionally substituted with one or more R⁸. In some embodiments, ring B is tetrahydrofuranyl, pyrrolidinyl, piperidinyl, piperazinyl, morpholinyl, or thiomorpholinyl, each of which is independently optionally substituted with one or more R⁸. In some embodiments, ring B is tetrahydrofuranyl or pyrrolidinyl, each of which is independently optionally substituted with one or more R. In some embodiments, ring B is ring B is a C₃₋₁₂ cycloalkyl or 3- to 12-membered heterocyclyl, each of which is optionally substituted with one or more R⁸. In some embodiments, ring B is cyclopentyl, tetrahydrofuranyl, or pyrrolidinyl, each of which is independently optionally substituted with one or more R⁸. In some embodiments, ring B is tetrahydrofuranyl, which is optionally substituted with one or more R⁸. In some embodiments, ring B is pyrrolidinyl, which is optionally substituted with one or more R⁸. In some embodiments, ring B is

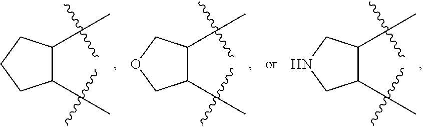 [SMILES: *C1CCCC1* ; *C1COCC1* ; *C1CNCC1*]

each of which is independently optionally substituted with one or more R⁸.

It is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (I) may be combined with every description, variation, embodiment or aspect of R², R³, R⁴, R⁵, R⁶, L¹, and L² the same as if each and every combination were specifically and individually listed. It is also understood that all descriptions, variations, embodiments or aspects of formula (I), where applicable, apply equally to other formulae detailed herein (e.g., any one of formulae (I-a), (I-b), (II), (II-a)-(II-b), (III), (III-a), (III-b), (IV), (IV-a), (IV-b), (V), (V-a)-(V-k), (VI), (VI-a), (VI-b), (VII), (VII-a), (VII-b)) and (VIII) and are equally described, the same as if each and every description, variation, embodiment or aspect were separately and individually listed for all formulae.

Exemplary compounds provided by the present disclosure are shown in Table 1. In some embodiments, provided is a compound selected from the compounds in Table 1, or a stereoisomer, tautomer, or a pharmaceutically acceptable salt of any of the foregoing. In some embodiments, provided is a compound selected from the compounds in Table 1, or a pharmaceutically acceptable salt thereof.

TABLE 1

| Cmpd # | Name | Cmpd # | Name |
|---|---|---|---|
| 1 | (2R,3S)-2-(4- | 2 | (2R,3S)-1-(2-chloropyrimidin-4-yl)-2-(4- |
| | (cyclopentylamino)phenyl)-N-(4- | | (cyclopentylamino)phenyl)-N-(4-methyl- |
| | methyl-3-(trifluoromethyl)phenyl)-1- | | 3-(trifluoromethyl)phenyl)piperidine-3- |
| | (thieno[2,3-c]pyridin-7-yl)piperidine-3- | | carboxamide |
| | carboxamide | | |
| 3 | (2R,3R)-2-(4- | 4 | (2R,3S)-2-(4-(cyclopentylamino)phenyl)- |
| | (cyclopentylamino)phenyl)-N-(4- | | N-(4-methyl-3-(trifluoromethyl)phenyl)- |
| | methyl-3-(trifluoromethyl)phenyl)-1- | | 1-(pyrimidin-4-yl)piperidine-3- |
| | (pyrimidin-4-yl)piperidine-3- | | carboxamide |
| | carboxamide | | |
| 5 | (2R,3S)-2-(4- | 6 | (2R,3S)-2-(4-(cyclopentylamino)phenyl)- |
| | (cyclopentylamino)phenyl)-N-(4- | | N-(4-methyl-3-(trifluoromethyl)phenyl)- |
| | methyl-3-(trifluoromethyl)phenyl)-1- | | 1-(1,7-naphthyridin-8-yl)piperidine-3- |
| | (quinazolin-4-yl)piperidine-3- | | carboxamide |
| | carboxamide | | |
| 7 | (2R,3R)-2-(4- | 8 | (2R,3S)-2-(4-(cyclopentylamino)phenyl)- |
| | (cyclopentylamino)phenyl)-N-(4- | | 1-((3,5-dimethylisoxazol-4-yl)sulfonyl)- |
| | methyl-3-(trifluoromethyl)phenyl)-1- | | N-(4-methyl-3- |
| | (pyrido[3,2-d]pyrimidin-4-yl)piperidine- | | (trifluoromethyl)phenyl)piperidine-3- |
| | 3-carboxamide | | carboxamide |
| 9 | (2R,3S)-2-(4- | 10 | (2R,3S)-2-(4-(cyclopentylamino)phenyl)- |
| | (cyclopentylamino)phenyl)-1-((2,4- | | 1-((2,5-dimethylphenyl)sulfonyl)-N-(4- |
| | dimethylphenyl)sulfonyl)-N-(4-methyl- | | methyl-3- |
| | 3-(trifluoromethyl)phenyl)piperidine-3- | | (trifluoromethyl)phenyl)piperidine-3- |
| | carboxamide | | carboxamide |
| 11 | (2R,3S)-2-(4- | 12 | (2R,3S)-2-(4-(cyclopentylamino)phenyl)- |
| | (cyclopentylamino)phenyl)-1-((2,6- | | 1-((3,5-dimethylphenyl)sulfonyl)-N-(4- |
| | dimethylphenyl)sulfonyl)-N-(4-methyl- | | methyl-3- |
| | 3-(trifluoromethyl)phenyl)piperidine-3- | | (trifluoromethyl)phenyl)piperidine-3- |
| | carboxamide | | carboxamide |
| 13 | (2R,3S)-2-(4- | 14 | (2R,3S)-2-(4-(cyclopentylamino)phenyl)- |
| | (cyclopentylamino)phenyl)-1- | | N-(4-methyl-3-(trifluoromethyl)phenyl)- |
| | (mesitylsulfonyl)-N-(4-methyl-3- | | 1-((2-nitrophenyl)sulfonyl)piperidine-3- |
| | (trifluoromethyl)phenyl)piperidine-3- | | carboxamide |
| | carboxamide | | |
| 15 | (2R,3S)-2-(4- | 16 | (2R,3S)-1-((3-chloro-2- |
| | (cyclopentylamino)phenyl)-1-((4-fluoro- | | methylphenyl)sulfonyl)-2-(4- |
| | 2-methylphenyl)sulfonyl)-N-(4-methyl- | | (cyclopentylamino)phenyl)-N-(4-methyl- |
| | 3-(trifluoromethyl)phenyl)piperidine-3- | | 3-(trifluoromethyl)phenyl)piperidine-3- |
| | carboxamide | | carboxamide |
| 17 | (2R,3S)-2-(4- | 18 | (2R,3S)-1-((3-fluoro-2- |
| | (cyclopentylamino)phenyl)-1-((5-fluoro- | | methylphenyl)sulfonyl)-2-(4- |
| | 2-methylphenyl)sulfonyl)-N-(4-methyl- | | (cyclopentylamino)phenyl)-N-(4-methyl-3- |
| | 3-(trifluoromethyl)phenyl)piperidine-3- | | (trifluoromethyl)phenyl)piperidine-3- |
| | carboxamide | | carboxamide |
| 19 | (2R,3S)-2-(4- | 20 | (2R,3S)-2-(4-(cyclopentylamino)phenyl)- |
| | (cyclopentylamino)phenyl)-1-((2,6- | | 1-((2,6-dichlorophenyl)sulfonyl)-N-(4- |
| | difluorophenyl)sulfonyl)-N-(4-methyl- | | methyl-3- |
| | 3-(trifluoromethyl)phenyl)piperidine-3- | | (trifluoromethyl)phenyl)piperidine-3- |
| | carboxamide | | carboxamide |
| 21 | methyl 2-(((2R,3S)-2-(4- | 22 | (2R,3S)-2-(4-(cyclopentylamino)phenyl)- |
| | (cyclopentylamino)phenyl)-3-((4- | | N-(4-methyl-3-(trifluoromethyl)phenyl)- |
| | methyl-3- | | 1-(o-tolylsulfonyl)piperidine-3- |
| | (trifluoromethyl)phenyl)carbamoyl)piperidin- | | carboxamide |
| | 1-yl)sulfonyl)-3-methylbenzoate | | |
| 23 | (2R,3S)-2-(4- | 24 | (2R,3S)-2-(4-(cyclopentylamino)phenyl)- |
| | (cyclopentylamino)phenyl)-1-((2- | | N-(4-methyl-3-(trifluoromethyl)phenyl)- |
| | methoxyphenyl)sulfonyl)-N-(4-methyl- | | 1-((2- |
| | 3-(trifluoromethyl)phenyl)piperidine-3- | | (trifluoromethoxy)phenyl)sulfonyl)piperidine- |
| | carboxamide | | 3-carboxamide |
| 25 | (2R,3S)-2-(4- | 26 | (2R,3S)-1-((2-chlorophenyl)sulfonyl)-2- |
| | (cyclopentylamino)phenyl)-1-((2- | | (4-(cyclopentylamino)phenyl)-N-(4- |
| | fluorophenyl)sulfonyl)-N-(4-methyl-3- | | methyl-3- |
| | (trifluoromethyl)phenyl)piperidine-3- | | (trifluoromethyl)phenyl)piperidine-3- |
| | carboxamide | | carboxamide |
| 27 | (2R,3S)-1-((2-bromophenyl)sulfonyl)-2- | 28 | (2R,3S)-2-(4-(cyclopentylamino)phenyl)- |
| | (4-(cyclopentylamino)phenyl)-N-(4- | | N-(4-methyl-3-(trifluoromethyl)phenyl)- |
| | methyl-3- | | 1-((2- |
| | (trifluoromethyl)phenyl)piperidine-3- | | (trifluoromethyl)phenyl)sulfonyl)piperidine- |
| | carboxamide | | 3-carboxamide |
| 29 | (2R,3S)-2-(4- | 30 | (2R,3S)-1-((2-cyanophenyl)sulfonyl)-2- |
| | (cyclopentylamino)phenyl)-N-(4- | | (4-(cyclopentylamino)phenyl)-N-(4- |
| | methyl-3-(trifluoromethyl)phenyl)-1- | | methyl-3- |
| | ((2- | | (trifluoromethyl)phenyl)piperidine-3- |
| | (methylsulfonyl)phenyl)sulfonyl)piperidine- | | carboxamide |
| | 3-carboxamide | | |
| 31 | methyl 2-(((2R,3S)-2-(4- | 32 | (2R,3S)-2-(4-(cyclopentylamino)phenyl)- |
| | (cyclopentylamino)phenyl)-3-((4- | | N-(4-methyl-3-(trifluoromethyl)phenyl)- |
| | methyl-3- | | 1-(naphthalen-2-ylsulfonyl)piperidine-3- |
| | (trifluoromethyl)phenyl)carbamoyl)piperidin- | | carboxamide |
| | 1-yl)sulfonyl)benzoate | | |
| 33 | (2R,3S)-2-(4- | 34 | (2R,3S)-2-(4-(cyclopentylamino)phenyl)- |
| | (cyclopentylamino)phenyl)-N-(4- | | N-(4-methyl-3-(trifluoromethyl)phenyl)- |
| | methyl-3-(trifluoromethyl)phenyl)-1- | | 1-(phenylsulfonyl)piperidine-3- |
| | (naphthalen-1-ylsulfonyl)piperidine-3- | | carboxamide |
| | carboxamide | | |
| 35 | (2R,3S)-2-(4- | 36 | (2R,3S)-1-((2-chloropyridin-3- |
| | (cyclopentylamino)phenyl)-N-(4- | | yl)sulfonyl)-2-(4- |
| | methyl-3-(trifluoromethyl)phenyl)-1- | | (cyclopentylamino)phenyl)-N-(4-methyl- |
| | (pyridin-3-ylsulfonyl)piperidine-3- | | 3-(trifluoromethyl)phenyl)piperidine-3- |
| | carboxamide | | carboxamide |
| 37 | (2R,3S)-2-(4- | 38 | (2R,3S)-2-(4-(cyclopentylamino)phenyl)- |
| | (cyclopentylamino)phenyl)-N-(4- | | N-(4-methyl-3-(trifluoromethyl)phenyl)- |
| | methyl-3-(trifluoromethyl)phenyl)-1- | | 1-((1,3,5-trimethyl-1H-pyrazol-4- |
| | ((perfluorophenyl)sulfonyl)piperidine-3- | | yl)sulfonyl)piperidine-3-carboxamide |
| | carboxamide | | |
| 39 | (2R,3S,4aS,7aS)-2-(4- | 40 | (2R,3S)-1-(benzylsulfonyl)-2-(4- |
| | (cyclopentylamino)phenyl)-1-(2-fluoro- | | (cyclopentylamino)phenyl)-N-(4-methyl- |
| | 6-methylbenzoyl)-N-(4-methyl-3- | | 3-(trifluoromethyl)phenyl)piperidine-3- |
| | (trifluoromethyl)phenyl)octahydro-1H- | | carboxamide |
| | pyrrolo[3,4-b]pyridine-3-carboxamide | | |
| 41 | cis-4-(4-(cyclopentylamino)phenyl)-7- | 42 | 2-(4-(cyclopentylamino)phenyl)-1-(2- |
| | fluoro-N-(4-methyl-3- | | fluoro-6-methylbenzoyl)-5-hydroxy-N-(4- |
| | (trifluoromethyl)phenyl)-6-oxo- | | methyl-3- |
| | 1,2,3,4,6,11,12,12a- | | (trifluoromethyl)phenyl)piperidine-3- |
| | octahydrobenzo[e]pyrido[1,2-a]azepine- | | carboxamide |
| | 3-carboxamide | | |
| 43 | cis-4-(4-(cyclopentylamino)phenyl)-N- | 44 | (3S,4R)-4-(4-(cyclopentylamino)phenyl)- |
| | (4-methyl-3-(trifluoromethyl)phenyl)-6- | | N-(4-methyl-3-(trifluoromethyl)phenyl)- |
| | oxo-1,3,4,6,11,11a-hexahydro-2H- | | 6-oxo-1,2,3,4,6,11,12,12a- |
| | pyrido[1,2-b]isoquinoline-3- | | octahydrobenzo[e]pyrido[1,2-a]azepine-3- |
| | carboxamide | | carboxamide |
| 45 | (3R,4S)-4-(4- | 46 | cis-2-(4-(cyclopentylamino)phenyl)-1-(2- |
| | (cyclopentylamino)phenyl)-N-(4- | | fluoro-6-methylbenzoyl)-N-(3- |
| | methyl-3-(trifluoromethyl)phenyl)-6- | | (trifluoromethyl)phenyl)octahydro-1H- |
| | oxo-1,2,3,4,6,11,12,12a- | | cyclopenta[b]pyridine-3-carboxamide |
| | octahydrobenzo[e]pyrido[1,2-a]azepine- | | |
| | 3-carboxamide | | |
| 47 | (2R,3S,4aR,7aR)-2-(4- | 48 | (2S,3R,4aS,7aS)-2-(4- |
| | (cyclopentylamino)phenyl)-1-(2-fluoro- | | (cyclopentylamino)phenyl)-1-(2-fluoro-6- |
| | 6-methylbenzoyl)-N-(4-methyl-3- | | methylbenzoyl)-N-(4-methyl-3- |
| | (trifluoromethyl)phenyl)octahydro-1H- | | (trifluoromethyl)phenyl)octahydro-1H- |
| | cyclopenta[b]pyridine-3-carboxamide | | cyclopenta[b]pyridine-3-carboxamide |
| 49 | (2R,3S,4aR,7aR)-2-(4- | 50 | (2R,3S,4aR,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(1-methyl-1H- | | methylbenzoyl)-N-(1-methyl-1H-indazol- |
| | indazol-5-yl)octahydro-1H- | | 6-yl)octahydro-1H-cyclopenta[b]pyridine- |
| | cyclopenta[b]pyridine-3-carboxamide | | 3-carboxamide |
| 51 | (2R,3S,4aR,7aR)-2-(4- | 52 | (2R,3S,4aR,7aR)-2-(4- |
| | (cyclopentylamino)phenyl)-N-(4- | | (cyclopentylamino)phenyl)-1-(2-fluoro-6- |
| | (dimethylamino)phenyl)-1-(2-fluoro-6- | | methylbenzoyl)-N-(tetrahydro-2H-pyran- |
| | methylbenzoyl)octahydro-1H- | | 4-yl)octahydro-1H-cyclopenta[b]pyridine- |
| | cyclopenta[b]pyridine-3-carboxamide | | 3-carboxamide |
| 53 | (2R,3S,4aR,7aR)-2-(4- | 54 | (2R,3S,4aR,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(1- | | methylbenzoyl)-N-(1-methyl-1H-pyrazol- |
| | methylpiperidin-4-yl)octahydro-1H- | | 4-yl)octahydro-1H-cyclopenta[b]pyridine- |
| | cyclopenta[b]pyridine-3-carboxamide | | 3-carboxamide |
| 55 | cis-N-(3-chlorophenyl)-2-(4- | 56 | cis-2-(4-(cyclopentylamino)phenyl)-1-(2- |
| | (cyclopentylamino)phenyl)-1-(2-fluoro- | | fluoro-6-methylbenzoyl)-N-(3- |
| | 6-methylbenzoyl)octahydro-1H- | | fluorophenyl)octahydro-1H- |
| | cyclopenta[b]pyridine-3-carboxamide | | cyclopenta[b]pyridine-3-carboxamide |
| 57 | cis-2-(4-(cyclopentylamino)phenyl)-1- | 58 | cis-2-(4-(cyclopentylamino)phenyl)-1-(2- |
| | (2-fluoro-6-methylbenzoyl)-N-(pyridin- | | fluoro-6-methylbenzoyl)-N-(2- |
| | 3-yl)octahydro-1H- | | methylpyrimidin-5-yl)octahydro-1H- |
| | cyclopenta[b]pyridine-3-carboxamide | | cyclopenta[b]pyridine-3-carboxamide |
| 59 | cis-2-(4-(cyclopentylamino)phenyl)-1- | 60 | cis-N-(4-chloro-3- |
| | (2-fluoro-6-methylbenzoyl)-N-(1- | | (trifluoromethyl)phenyl)-2-(4- |
| | (oxetan-3-yl)-1H-indazol-6- | | (cyclopentylamino)phenyl)-1-(2-fluoro-6- |
| | yl)octahydro-1H-cyclopenta[b]pyridine- | | methylbenzoyl)octahydro-1H- |
| | 3-carboxamide | | cyclopenta[b]pyridine-3-carboxamide |
| 61 | cis-2-(4-(cyclopentylamino)phenyl)-N- | 62 | cis-N-(3-cyano-4-methylphenyl)-2-(4- |
| | (4-fluoro-3-(trifluoromethyl)phenyl)-1- | | (cyclopentylamino)phenyl)-1-(2-fluoro-6- |
| | (2-fluoro-6-methylbenzoyl)octahydro- | | methylbenzoyl)octahydro-1H- |
| | 1H-cyclopenta[b]pyridine-3- | | cyclopenta[b]pyridine-3-carboxamide |
| | carboxamide | | |
| 63 | cis-2-(4-(cyclopentylamino)phenyl)-1- | 64 | cis-2-(4-(cyclopentylamino)phenyl)-N- |
| | (2-fluoro-6-methylbenzoyl)-N-(6- | | (3,4-dichlorophenyl)-1-(2-fluoro-6- |
| | methylpyridin-3-yl)octahydro-1H- | | methylbenzoyl)octahydro-1H- |
| | cyclopenta[b]pyridine-3-carboxamide | | cyclopenta[b]pyridine-3-carboxamide |
| 65 | cis-2-(4-(cyclopentylamino)phenyl)-N- | 66 | cis-2-(4-(cyclopentylamino)phenyl)-1-(2- |
| | (3,4-difluorophenyl)-1-(2-fluoro-6- | | fluoro-6-methylbenzoyl)-N-(1-methyl- |
| | methylbenzoyl)octahydro-1H- | | 1H-indazol-6-yl)octahydro-1H- |
| | cyclopenta[b]pyridine-3-carboxamide | | cyclopenta[b]pyridine-3-carboxamide |
| 67 | cis-N-(benzo[d]oxazol-6-yl)-2-(4- | 68 | cis-2-(4-(cyclopentylamino)phenyl)-1-(2- |
| | (cyclopentylamino)phenyl)-1-(2-fluoro- | | fluoro-6-methylbenzoyl)-N-(4- |
| | 6-methylbenzoyl)octahydro-1H- | | formamido-3-hydroxyphenyl)octahydro- |
| | cyclopenta[b]pyridine-3-carboxamide | | 1H-cyclopenta[b]pyridine-3-carboxamide |
| 69 | cis-N-(benzo[d]thiazol-6-yl)-2-(4- | 70 | cis-2-(4-(cyclopentylamino)phenyl)-1-(2- |
| | (cyclopentylamino)phenyl)-1-(2-fluoro- | | fluoro-6-methylbenzoyl)-N-(3- |
| | 6-methylbenzoyl)octahydro-1H- | | (methylsulfonyl)phenyl)octahydro-1H- |
| | cyclopenta[b]pyridine-3-carboxamide | | cyclopenta[b]pyridine-3-carboxamide |
| 71 | cis-2-(4-(cyclopentylamino)phenyl)-N- | 72 | (cis-3-(6-chloro-1,2,3,4- |
| | (2,3-dihydrobenzo[b][1,4]dioxin-6-yl)- | | tetrahydroisoquinoline-2-carbonyl)-2-(4- |
| | 1-(2-fluoro-6-methylbenzoyl)octahydro- | | (cyclopentylamino)phenyl)octahydro-1H- |
| | 1H-cyclopenta[b]pyridine-3- | | cyclopenta[b]pyridin-1-yl)(2-fluoro-6- |
| | carboxamide | | methylphenyl)methanone |
| 73 | cis-2-(4-(cyclopentylamino)phenyl)-1- | 74 | cis-2-(4-(cyclopentylamino)phenyl)-1-(2- |
| | (2-fluoro-6-methylbenzoyl)-N- | | fluoro-6-methylbenzoyl)-N-(1-methyl- |
| | (quinolin-7-yl)octahydro-1H- | | 1H-benzo[d]imidazol-6-yl)octahydro-1H- |
| | cyclopenta[b]pyridine-3-carboxamide | | cyclopenta[b]pyridine-3-carboxamide |
| 75 | cis-N-(5-chloro-6-(2H-1,2,3-triazol-2- | 76 | cis-2-(4-(cyclopentylamino)phenyl)-N-(3- |
| | yl)pyridin-3-yl)-2-(4- | | (dimethylphosphoryl)-4-methylphenyl)-1- |
| | (cyclopentylamino)phenyl)-1-(2-fluoro- | | (2-fluoro-6-methylbenzoyl)octahydro-1H- |
| | 6-methylbenzoyl)octahydro-1H- | | cyclopenta[b]pyridine-3-carboxamide |
| | cyclopenta[b]pyridine-3-carboxamide | | |
| 77 | cis-2-(4-(cyclopentylamino)phenyl)-1- | 78 | cis-2-(4-(cyclopentylamino)phenyl)-1-(2- |
| | (2-fluoro-6-methylbenzoyl)-N-(2- | | fluoro-6-methylbenzoyl)-N-(1-methyl- |
| | methyl-1,2,3,4-tetrahydroisoquinolin-6- | | 1H-pyrazolo[4,3-b]pyridin-6- |
| | yl)octahydro-1H-cyclopenta[b]pyridine- | | yl)octahydro-1H-cyclopenta[b]pyridine-3- |
| | 3-carboxamide | | carboxamide |
| 79 | cis-2-(4-(cyclopentylamino)phenyl)-1- | 80 | (2R,3S,4aR,7aR)-2-(4- |
| | (2-fluoro-6-methylbenzoyl)-N-(2- | | (cyclopentylamino)phenyl)-1-(2-fluoro-6- |
| | (trifluoromethyl)pyridin-4-yl)octahydro- | | methylbenzoyl)-N-(1-(pyridin-2- |
| | 1H-cyclopenta[b]pyridine-3- | | ylmethyl)-1H-indazol-5-yl)octahydro-1H- |
| | carboxamide | | cyclopenta[b]pyridine-3-carboxamide |
| 81 | (2R,3S,4aR,7aR)-2-(4- | 82 | (2R,3S,4aR,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(1-(tetrahydro-2H- | | methylbenzoyl)-N-(1-(1-methylpiperidin- |
| | pyran-4-yl)-1H-indazol-5-yl)octahydro- | | 4-yl)-1H-indazol-5-yl)octahydro-1H- |
| | 1H-cyclopenta[b]pyridine-3- | | cyclopenta[b]pyridine-3-carboxamide |
| | carboxamide | | |
| 83 | (2R,3S,4aR,7aR)-2-(4- | 84 | (2R,3S,4aR,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(1-(oxetan-3-yl)- | | methylbenzoyl)-N-(1H-indazol-5- |
| | 1H-indazol-5-yl)octahydro-1H- | | yl)octahydro-1H-cyclopenta[b]pyridine-3- |
| | cyclopenta[b]pyridine-3-carboxamide | | carboxamide |
| 85 | (2R,3S,4aR,7aR)-2-(4- | 86 | (2R,3S,4aR,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(1-methyl-1H- | | methylbenzoyl)-N-(1-(pyridin-3- |
| | indol-5-yl)octahydro-1H- | | ylmethyl)-1H-indazol-5-yl)octahydro-1H- |
| | cyclopenta[b]pyridine-3-carboxamide | | cyclopenta[b]pyridine-3-carboxamide |
| 87 | (2R,3S,4aR,7aR)-2-(4- | 88 | cis-1-(2-fluoro-6-methylbenzoyl)-N-(4- |
| | (cyclopentylamino)phenyl)-1-(2-fluoro- | | methyl-3-(trifluoromethyl)phenyl)-2-(4- |
| | 6-methylbenzoyl)-N-(1-(pyridin-4- | | ((tetrahydro-2H-pyran-4- |
| | ylmethyl)-1H-indazol-5-yl)octahydro- | | yl)amino)phenyl)octahydro-1H- |
| | 1H-cyclopenta[b]pyridine-3- | | cyclopenta[b]pyridine-3-carboxamide |
| | carboxamide | | |
| 89 | (2R,3S,4aR,7aR)-1-(2-fluoro-6- | 90 | cis-1-(2-fluoro-6-methylbenzoyl)-N- |
| | methylbenzoyl)-N-(1-methyl-1H- | | (quinolin-7-yl)-2-(4-((tetrahydro-2H-pyran-4- |
| | indazol-5-yl)-2-(4-((tetrahydro-2H- | | yl)amino)phenyl)octahydro-1H- |
| | pyran-4-yl)amino)phenyl)octahydro-1H- | | cyclopenta[b]pyridine-3-carboxamide |
| | cyclopenta[b]pyridine-3-carboxamide | | |
| 91 | (2S,3R,4aS,7aS)-1-(2-fluoro-6- | 92 | (2R,3S,4aR,7aR)-1-(2-fluoro-6- |
| | methylbenzoyl)-N-(1-methyl-1H- | | methylbenzoyl)-N-(1-(pyridin-2- |
| | indazol-5-yl)-2-(4-((tetrahydro-2H- | | ylmethyl)-1H-indazol-5-yl)-2-(4- |
| | pyran-4-yl)amino)phenyl)octahydro-1H- | | ((tetrahydro-2H-pyran-4- |
| | cyclopenta[b]pyridine-3-carboxamide | | yl)amino)phenyl)octahydro-1H- |
| | | | cyclopenta[b]pyridine-3-carboxamide |
| 93 | (2R,3S,4aR,7aR)-N-(1-(1-chloro-3- | 94 | (2R,3S,4aR,7aR)-N-(4- |
| | hydroxypropan-2-yl)-1H-indazol-5-yl)- | | (dimethylamino)phenyl)-1-(2-fluoro-6- |
| | 1-(2-fluoro-6-methylbenzoyl)-2-(4- | | methylbenzoyl)-2-(4-((tetrahydro-2H- |
| | ((tetrahydro-2H-pyran-4- | | pyran-4-yl)amino)phenyl)octahydro-1H- |
| | yl)amino)phenyl)octahydro-1H- | | cyclopenta[b]pyridine-3-carboxamide |
| | cyclopenta[b]pyridine-3-carboxamide | | |
| 95 | (2R,3S,4aR,7aR)-1-(2-fluoro-6- | 96 | (2R,3S,4aR,7aR)-1-(2-fluoro-6- |
| | methylbenzoyl)-N-(quinolin-6-yl)-2-(4- | | methylbenzoyl)-N-(1H-indazol-5-yl)-2- |
| | ((tetrahydro-2H-pyran-4- | | (4-((tetrahydro-2H-pyran-4- |
| | yl)amino)phenyl)octahydro-1H- | | yl)amino)phenyl)octahydro-1H- |
| | cyclopenta[b]pyridine-3-carboxamide | | cyclopenta[b]pyridine-3-carboxamide |
| 97 | (2R,3S,4aR,7aR)-1-(2-fluoro-6- | 98 | (2R,3S,4aR,7aR)-1-(2-fluoro-6- |
| | methylbenzoyl)-N-(2-methyl-2H- | | methylbenzoyl)-N-(1-methyl-1H-indol-5- |
| | indazol-5-yl)-2-(4-((tetrahydro-2H- | | yl)-2-(4-((tetrahydro-2H-pyran-4- |
| | pyran-4-yl)amino)phenyl)octahydro-1H- | | yl)amino)phenyl)octahydro-1H- |
| | cyclopenta[b]pyridine-3-carboxamide | | cyclopenta[b]pyridine-3-carboxamide |
| 99 | (2R,3S,4aR,7aR)-1-(2-fluoro-6- | 100 | (2R,3S,4aR,7aR)-1-(2-fluoro-6- |
| | methylbenzoyl)-N-(1-(oxetan-3-yl)-1H- | | methylbenzoyl)-N-(1-(1-methylpiperidin- |
| | indazol-5-yl)-2-(4-((tetrahydro-2H- | | 4-yl)-1H-indazol-5-yl)-2-(4-((tetrahydro- |
| | pyran-4-yl)amino)phenyl)octahydro-1H- | | 2H-pyran-4-yl)amino)phenyl)octahydro- |
| | cyclopenta[b]pyridine-3-carboxamide | | 1H-cyclopenta[b]pyridine-3-carboxamide |
| 101 | (2R,3S,4aR,7aR)-1-(2-fluoro-6- | 102 | (2R,3S,4aR,7aR)-1-(2-fluoro-6- |
| | methylbenzoyl)-N-(1-(pyridin-4- | | methylbenzoyl)-N-(1-(2-hydroxyethyl)- |
| | ylmethyl)-1H-indazol-5-yl)-2-(4- | | 1H-indazol-5-yl)-2-(4-((tetrahydro-2H- |
| | ((tetrahydro-2H-pyran-4- | | pyran-4-yl)amino)phenyl)octahydro-1H- |
| | yl)amino)phenyl)octahydro-1H- | | cyclopenta[b]pyridine-3-carboxamide |
| | cyclopenta[b]pyridine-3-carboxamide | | |
| 103 | (2R,3S,4aR,7aR)-N-(1-(2- | 104 | (2R,3S,4aR,7aR)-1-(2-fluoro-6- |
| | (dimethylamino)ethyl)-1H-indazol-5- | | methylbenzoyl)-N-(2-(2-hydroxyethyl)- |
| | yl)-1-(2-fluoro-6-methylbenzoyl)-2-(4- | | 2H-indazol-5-yl)-2-(4-((tetrahydro-2H- |
| | ((tetrahydro-2H-pyran-4- | | pyran-4-yl)amino)phenyl)octahydro-1H- |
| | yl)amino)phenyl)octahydro-1H- | | cyclopenta[b]pyridine-3-carboxamide |
| | cyclopenta[b]pyridine-3-carboxamide | | |
| 105 | (2R,3S,4aR,7aR)-N-(2-(2- | 106 | (2R,3S,4aR,7aR)-1-(2-fluoro-6- |
| | (dimethylamino)ethyl)-2H-indazol-5- | | methylbenzoyl)-N-(1-(tetrahydro-2H- |
| | yl)-1-(2-fluoro-6-methylbenzoyl)-2-(4- | | pyran-4-yl)-1H-indazol-5-yl)-2-(4- |
| | ((tetrahydro-2H-pyran-4- | | ((tetrahydro-2H-pyran-4- |
| | yl)amino)phenyl)octahydro-1H- | | yl)amino)phenyl)octahydro-1H- |
| | cyclopenta[b]pyridine-3-carboxamide | | cyclopenta[b]pyridine-3-carboxamide |
| 107 | (2R,3S,4aR,7aR)-1-(2-fluoro-6- | 108 | (2R,3S,4aR,7aR)-N-(1- |
| | methylbenzoyl)-N-(1-(pyridin-3- | | (cyclopropylmethyl)-1H-indazol-5-yl)-1- |
| | ylmethyl)-1H-indazol-5-yl)-2-(4- | | (2-fluoro-6-methylbenzoyl)-2-(4- |
| | ((tetrahydro-2H-pyran-4- | | ((tetrahydro-2H-pyran-4- |
| | yl)amino)phenyl)octahydro-1H- | | yl)amino)phenyl)octahydro-1H- |
| | cyclopenta[b]pyridine-3-carboxamide | | cyclopenta[b]pyridine-3-carboxamide |
| 109 | (2R,3S,4aR,7aR)-1-(2-fluoro-6- | 110 | cis-2-(4-(cyclopentylamino)phenyl)-N-(4- |
| | methylbenzoyl)-N-(1-(2-fluoroethyl)- | | methyl-3-(trifluoromethyl)phenyl)-1- |
| | 1H-indazol-5-yl)-2-(4-((tetrahydro-2H- | | (oxazole-4-carbonyl)octahydro-1H- |
| | pyran-4-yl)amino)phenyl)octahydro-1H- | | cyclopenta[b]pyridine-3-carboxamide |
| | cyclopenta[b]pyridine-3-carboxamide | | |
| 111 | cis-2-(4-(cyclopentylamino)phenyl)-N- | 112 | cis-2-(4-(cyclopentylamino)phenyl)-1-(1- |
| | (4-methyl-3-(trifluoromethyl)phenyl)-1- | | methyl-1H-pyrazole-4-carbonyl)-N-(4- |
| | (tetrahydro-2H-pyran-4- | | methyl-3- |
| | carbonyl)octahydro-1H- | | (trifluoromethyl)phenyl)octahydro-1H- |
| | cyclopenta[b]pyridine-3-carboxamide | | cyclopenta[b]pyridine-3-carboxamide |
| 113 | cis-2-(4-(cyclopentylamino)phenyl)-1- | 114 | cis-2-(4-(cyclopentylamino)phenyl)-N-(4- |
| | (1-methyl-1H-imidazole-4-carbonyl)-N- | | methyl-3-(trifluoromethyl)phenyl)-1- |
| | (4-methyl-3- | | (thiazole-4-carbonyl)octahydro-1H- |
| | (trifluoromethyl)phenyl)octahydro-1H- | | cyclopenta[b]pyridine-3-carboxamide |
| | cyclopenta[b]pyridine-3-carboxamide | | |
| 115 | cis-2-(4-(cyclopentylamino)phenyl)-N- | 116 | (2R,3S,4aR,7aR)-1-(2-fluoro-6- |
| | (4-methyl-3-(trifluoromethyl)phenyl)-1- | | methylbenzoyl)-N-(1-methyl-1H-indazol- |
| | (pyrimidine-5-carbonyl)octahydro-1H- | | 5-yl)-2-(4-(((R)-2- |
| | cyclopenta[b]pyridine-3-carboxamide | | (trifluoromethyl)pyrrolidin-1- |
| | | | yl)methyl)phenyl)octahydro-1H- |
| | | | cyclopenta[b]pyridine-3-carboxamide |
| 117 | cis-1-(2-fluoro-6-methylbenzoyl)-N-(1- | 118 | cis-2-(4-(cyclopentylamino)phenyl)-1-(2- |
| | methyl-1H-indazol-5-yl)-2-(4-((1- | | fluoro-6-methylbenzoyl)-N-(4-methyl-3- |
| | methylpiperidin-4- | | (trifluoromethyl)phenyl)octahydrofuro[3,4- |
| | yl)amino)phenyl)octahydro-1H- | | b]pyridine-3-carboxamide |
| | cyclopenta[b]pyridine-3-carboxamide | | |
| 119 | (2R,3S,4aR,7aS)-2-(4- | 120 | (2S,3R,4aS,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(4-methyl-3- | | methylbenzoyl)-N-(4-methyl-3- |
| | (trifluoromethyl)phenyl)octahydrofuro[3,4- | | (trifluoromethyl)phenyl)octahydrofuro[3,4- |
| | b]pyridine-3-carboxamide | | b]pyridine-3-carboxamide |
| 121 | cis-2-(4-(cyclopentylamino)phenyl)-1- | 122 | (2R,3S,4aR,7aS)-2-(4- |
| | (2-fluoro-6-methylbenzoyl)-N-(1- | | (cyclopentylamino)phenyl)-1-(2-fluoro-6- |
| | methyl-1H-indazol-5- | | methylbenzoyl)-N-(1-methyl-1H-indazol- |
| | yl)octahydrofuro[3,4-b]pyridine-3- | | 6-yl)octahydrofuro[3,4-b]pyridine-3- |
| | carboxamide | | carboxamide |
| 123 | (2R,3S,4aR,7aS)-2-(4- | 124 | (2R,3S,4aR,7aS)-2-(4- |
| | (cyclopentylamino)phenyl)-1-(2-fluoro- | | (cyclopentylamino)phenyl)-N-(4- |
| | 6-methylbenzoyl)-N-(1-methyl-1H- | | (dimethylamino)phenyl)-1-(2-fluoro-6- |
| | pyrazolo[4,3-b]pyridin-6- | | methylbenzoyl)octahydrofuro[3,4- |
| | yl)octahydrofuro[3,4-b]pyridine-3- | | b]pyridine-3-carboxamide |
| | carboxamide | | |
| 125 | (2S,3R,4aS,7aR)-2-(4- | 126 | (2R,3S,4aR,7aS)-2-(4- |
| | (cyclopentylamino)phenyl)-N-(4- | | (cyclopentylamino)phenyl)-1-(2-fluoro-6- |
| | (dimethylamino)phenyl)-1-(2-fluoro-6- | | methylbenzoyl)-N-(1-methyl-1H-indazol- |
| | methylbenzoyl)octahydrofuro[3,4- | | 5-yl)octahydrofuro[3,4-b]pyridine-3- |
| | b]pyridine-3-carboxamide | | carboxamide |
| 127 | cis-1-(2-fluoro-6-methylbenzoyl)-N-(1- | 128 | cis-1-(2-fluoro-6-methylbenzoyl)-N- |
| | methyl-1H-pyrazol-4-yl)-2-(4- | | (pyridin-3-yl)-2-(4-((tetrahydro-2H- |
| | ((tetrahydro-2H-pyran-4- | | pyran-4- |
| | yl)amino)phenyl)octahydrofuro[3,4- | | yl)amino)phenyl)octahydrofuro[3,4- |
| | b]pyridine-3-carboxamide | | b]pyridine-3-carboxamide |
| 129 | cis-1-(2-fluoro-6-methylbenzoyl)-N- | 130 | cis-N-(3-(dimethylphosphoryl)-4- |
| | phenyl-2-(4-((tetrahydro-2H-pyran-4- | | methylphenyl)-1-(2-fluoro-6- |
| | yl)amino)phenyl)octahydrofuro[3,4- | | methylbenzoyl)-2-(4-((tetrahydro-2H- |
| | b]pyridine-3-carboxamide | | pyran-4- |
| | | | yl)amino)phenyl)octahydrofuro[3,4- |
| | | | b]pyridine-3-carboxamide |
| 131 | cis-N-(benzo[d]oxazol-6-yl)-1-(2- | 132 | cis-N-(3-cyano-4-methylphenyl)-1-(2- |
| | fluoro-6-methylbenzoyl)-2-(4- | | fluoro-6-methylbenzoyl)-2-(4- |
| | ((tetrahydro-2H-pyran-4- | | ((tetrahydro-2H-pyran-4- |
| | yl)amino)phenyl)octahydrofuro[3,4- | | yl)amino)phenyl)octahydrofuro[3,4- |
| | b]pyridine-3-carboxamide | | b]pyridine-3-carboxamide |
| 133 | cis-1-(2-fluoro-6-methylbenzoyl)-N-(2- | 134 | cis-1-(2-fluoro-6-methylbenzoyl)-N-(1- |
| | methylpyrimidin-5-yl)-2-(4- | | methyl-1H-indazol-5-yl)-2-(4- |
| | ((tetrahydro-2H-pyran-4- | | ((tetrahydro-2H-pyran-4- |
| | yl)amino)phenyl)octahydrofuro[3,4- | | yl)amino)phenyl)octahydrofuro[3,4- |
| | b]pyridine-3-carboxamide | | b]pyridine-3-carboxamide |
| 135 | cis-1-(2-fluoro-6-methylbenzoyl)-N-(4- | 136 | cis-N-(4-(dimethylamino)phenyl)-1-(2- |
| | methyl-3-(trifluoromethyl)phenyl)-2-(4- | | fluoro-6-methylbenzoyl)-2-(4- |
| | ((tetrahydro-2H-pyran-4- | | ((tetrahydro-2H-pyran-4- |
| | yl)amino)phenyl)octahydrofuro[3,4- | | yl)amino)phenyl)octahydrofuro[3,4- |
| | b]pyridine-3-carboxamide | | b]pyridine-3-carboxamide |
| 137 | (2R,3S,4aR,7aS)-2-(4- | 138 | (2R,3S,4aR,7aS)-2-(4- |
| | (cyclopentylamino)phenyl)-1-(2-fluoro- | | (cyclopentylamino)phenyl)-1-(2-fluoro-6- |
| | 6-methylbenzoyl)-N-(1-(2- | | methylbenzoyl)-N-(1H-indazol-5- |
| | hydroxyethyl)-1H-indazol-5- | | yl)octahydrofuro[3,4-b]pyridine-3- |
| | yl)octahydrofuro[3,4-b]pyridine-3- | | carboxamide |
| | carboxamide | | |
| 139 | (2R,3S,4aR,7aS)-2-(4- | 140 | (2S,3R,4aS,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(1-methyl-1H- | | methylbenzoyl)-N-(1-methyl-1H-indazol- |
| | indol-5-yl)octahydrofuro[3,4- | | 5-yl)octahydrofuro[3,4-b]pyridine-3- |
| | b]pyridine-3-carboxamide | | carboxamide |
| 141 | cis-2-(4-((3,3- | 142 | (2R,3S,4aR,7aS)-2-(4-((3,3- |
| | dimethylmorpholino)methyl)phenyl)-1- | | dimethylmorpholino)methyl)phenyl)-1-(2- |
| | (2-fluoro-6-methylbenzoyl)-N-(4- | | fluoro-6-methylbenzoyl)-N-(1-methyl- |
| | methyl-3- | | 1H-indazol-5-yl)octahydrofuro[3,4- |
| | (trifluoromethyl)phenyl)octahydrofuro[3,4- | | b]pyridine-3-carboxamide |
| | b]pyridine-3-carboxamide | | |
| 143 | cis-2-(4-(cyclopentylamino)phenyl)-1- | 144 | cis-2-(4- |
| | (2-fluoro-6-methylbenzoyl)-6-methyl- | | (cyclopentyl(methyl)amino)phenyl)-1-(2- |
| | N-(4-methyl-3- | | fluoro-6-methylbenzoyl)-6-methyl-N-(4- |
| | (trifluoromethyl)phenyl)octahydro-1H- | | methyl-3- |
| | pyrrolo[3,4-b]pyridine-3-carboxamide | | (trifluoromethyl)phenyl)octahydro-1H- |
| | | | pyrrolo[3,4-b]pyridine-3-carboxamide |
| 145 | (2R,3S,4aS,7aS)-2-(4- | 146 | (2R,3S,4aS,7aS)-6-acetyl-2-(4- |
| | (cyclopentylamino)phenyl)-1-(2-fluoro- | | (cyclopentylamino)phenyl)-1-(2-fluoro-6- |
| | 6-methylbenzoyl)-N-(4-methyl-3- | | methylbenzoyl)-N-(4-methyl-3- |
| | (trifluoromethyl)phenyl)-6-(2,2,2- | | (trifluoromethyl)phenyl)octahydro-1H- |
| | trifluoroethyl)octahydro-1H- | | pyrrolo[3,4-b]pyridine-3-carboxamide |
| | pyrrolo[3,4-b]pyridine-3-carboxamide | | |
| 147 | cis-2-(4-(cyclopentylamino)phenyl)-1- | 148 | (2R,3S,4aR,7aR)-1-(2-fluoro-6- |
| | (2-fluoro-6-methylbenzoyl)-N-(1- | | methylbenzoyl)-N-(1-methyl-1H-indazol- |
| | methyl-1H-indazol-5-yl)octahydro-1H- | | 5-yl)-2-(4-((2-oxopyrrolidin-1- |
| | cyclopenta[b]pyridine-3-carboxamide | | yl)methyl)phenyl)octahydro-1H- |
| | | | cyclopenta[b]pyridine-3-carboxamide |
| 149 | (2R,3S,4aR,7aR)-2-(4-((3,3- | 150 | (2R,3S,4aR,7aR)-2-(4-((3,3- |
| | difluoropyrrolidin-1-yl)methyl)phenyl)- | | dimethylmorpholino)methyl)phenyl)-1-(2- |
| | 1-(2-fluoro-6-methylbenzoyl)-N-(1- | | fluoro-6-methylbenzoyl)-N-(1-methyl- |
| | methyl-1H-indazol-5-yl)octahydro-1H- | | 1H-indazol-5-yl)octahydro-1H- |
| | cyclopenta[b]pyridine-3-carboxamide | | cyclopenta[b]pyridine-3-carboxamide |
| 151 | (2R,3S,4aR,7aR)-2-(4-((7-oxa-4- | 152 | (2R,3S)-2-(4-(cyclopentylamino)phenyl)- |
| | azaspiro[2.5]octan-4-yl)methyl)phenyl)- | | 1-(2-fluoro-6-methylbenzoyl)-N-(4- |
| | 1-(2-fluoro-6-methylbenzoyl)-N-(1- | | methyl-3-(trifluoromethyl)phenyl)-6- |
| | methyl-1H-indazol-5-yl)octahydro-1H- | | (oxetan-3-yl)octahydro-1H-pyrrolo[3,4- |
| | cyclopenta[b]pyridine-3-carboxamide | | b]pyridine-3-carboxamide |
| 153 | (2R,3S)-2-(4- | 154 | (2R,3S)-2-(4-(cyclopentylamino)phenyl)- |
| | (cyclopentylamino)phenyl)-6- | | 1-(2-fluoro-6-methylbenzoyl)-N-(4- |
| | cyclopropyl-1-(2-fluoro-6- | | methyl-3-(trifluoromethyl)phenyl)-6- |
| | methylbenzoyl)-N-(4-methyl-3- | | (tetrahydrofuran-3-yl)octahydro-1H- |
| | (trifluoromethyl)phenyl)octahydro-1H- | | pyrrolo[3,4-b]pyridine-3-carboxamide |
| | pyrrolo[3,4-b]pyridine-3-carboxamide | | |
| 155 | cis-2-(4-(cyclopentylamino)phenyl)-1- | 156 | (2R,3S)-2-(4-(cyclopentylamino)phenyl)- |
| | (2-fluoro-6-methylbenzoyl)-N-(4- | | 1-(2-fluoro-6-methylbenzoyl)-5-hydroxy- |
| | methyl-3- | | N-(4-methyl-3- |
| | (trifluoromethyl)phenyl)octahydro-1H- | | (trifluoromethyl)phenyl)piperidine-3- |
| | cyclopenta[b]pyridine-3-carboxamide | | carboxamide |
| 157 | (2S,3R,4aS,7aS)-2-(4- | 158 | cis-1-(2-fluoro-6-methylbenzoyl)-N-(1- |
| | (cyclopentylamino)phenyl)-1-(2-fluoro- | | methyl-1H-indazol-5-yl)-2-(4- |
| | 6-methylbenzoyl)-N-(1-methyl-1H- | | ((tetrahydro-2H-pyran-4- |
| | indazol-5-yl)octahydro-1H- | | yl)amino)phenyl)octahydro-1H- |
| | cyclopenta[b]pyridine-3-carboxamide | | cyclopenta[b]pyridine-3-carboxamide |
| 159 | (2R,3S)-2-(4- | 160 | ((2R,3R)-2-(4- |
| | (cyclopentylamino)phenyl)-1-(2-fluoro- | | (cyclopentylamino)phenyl)-3-(((4-methyl-3- |
| | 6-methylbenzyl)-N-(4-methyl-3- | | (trifluoromethyl)phenyl)amino)methyl)piperidin- |
| | (trifluoromethyl)phenyl)piperidine-3- | | 1-yl)(2-fluoro-6- |
| | carboxamide | | methylphenyl)methanone |
| 161 | (2R,3S)-2-(4- | 162 | (2R,3S)-2-(4-(cyclopentylamino)phenyl)- |
| | (cyclopentylamino)phenyl)-N-(4- | | N-(4-methyl-3-(trifluoromethyl)phenyl)- |
| | methyl-3-(trifluoromethyl)phenyl)-1- | | 1-(pyrido[3,4-d]pyrimidin-4- |
| | (pyrido[3,2-d]pyrimidin-4-yl)piperidine- | | yl)piperidine-3-carboxamide |
| | 3-carboxamide | | |
| 163 | benzyl cyclopentyl(4-((2R,3S)-3-((4- | 164 | benzyl cyclopentyl(4-((2R,3S)-3-((4- |
| | methyl-3- | | methyl-3- |
| | (trifluoromethyl)phenyl)carbamoyl)-1- | | (trifluoromethyl)phenyl)carbamoyl)-1- |
| | (quinazolin-4-yl)piperidin-2- | | (1,7-naphthyridin-8-yl)piperidin-2- |
| | yl)phenyl)carbamate | | yl)phenyl)carbamate |
| 165 | benzyl cyclopentyl(4-((2R,3S)-3-((4- | 166 | benzyl cyclopentyl(4-((2R,3S)-3-((4- |
| | methyl-3- | | methyl-3- |
| | (trifluoromethyl)phenyl)carbamoyl)-1- | | (trifluoromethyl)phenyl)carbamoyl)-1- |
| | (pyrido[3,4-b]pyrazin-5-yl)piperidin-2- | | (pyrido[3,2-d]pyrimidin-4-yl)piperidin-2- |
| | yl)phenyl)carbamate | | yl)phenyl)carbamate |
| 167 | benzyl cyclopentyl(4-((2R,3S)-3-((4- | 168 | (2R,3S)-2-(4-(cyclopentyl(1,7- |
| | methyl-3- | | naphthyridin-8-yl)amino)phenyl)-N-(4- |
| | (trifluoromethyl)phenyl)carbamoyl)-1- | | methyl-3- |
| | (pyrido[3,4-d]pyrimidin-4-yl)piperidin- | | (trifluoromethyl)phenyl)piperidine-3- |
| | 2-yl)phenyl)carbamate | | carboxamide |
| 169 | (2R,3S)-2-(4-(cyclopentyl(thiazolo[4,5- | 170 | (2S,3S)-1-(2-fluoro-6-methylbenzoyl)-N- |
| | c]pyridin-4-yl)amino)phenyl)-N-(4- | | (4-methyl-3-(trifluoromethyl)phenyl)-2- |
| | methyl-3- | | (2-oxaspiro[4.5]decan-8-yl)piperidine-3- |
| | (trifluoromethyl)phenyl)piperidine-3- | | carboxamide |
| | carboxamide | | |
| 171 | (2R,3R)-2-(4-(cyclopentyl(1,7- | 172 | (2R,3S)-2-(4-(cyclopentyl(pyrido[3,4- |
| | naphthyridin-8-yl)amino)phenyl)-N-(4- | | b]pyrazin-5-yl)amino)phenyl)-N-(4- |
| | methyl-3- | | methyl-3- |
| | (trifluoromethyl)phenyl)piperidine-3- | | (trifluoromethyl)phenyl)piperidine-3- |
| | carboxamide | | carboxamide |
| 173 | (2R,3S)-2-(4-(cyclopentyl(thieno[2,3- | 174 | (2R,3R)-2-(4-(cyclopentyl(pyrido[3,2- |
| | c]pyridin-7-yl)amino)phenyl)-N-(4- | | d]pyrimidin-4-yl)amino)phenyl)-N-(4- |
| | methyl-3- | | methyl-3- |
| | (trifluoromethyl)phenyl)piperidine-3- | | (trifluoromethyl)phenyl)piperidine-3- |
| | carboxamide | | carboxamide |
| 175 | (2R,3S)-2-(4-(cyclopentyl(isoquinolin- | 176 | (2R,3S)-2-(4-(cyclopentyl(pyrido[3,2- |
| | 1-yl)amino)phenyl)-N-(4-methyl-3- | | d]pyrimidin-4-yl)amino)phenyl)-N-(4- |
| | (trifluoromethyl)phenyl)piperidine-3- | | methyl-3- |
| | carboxamide | | (trifluoromethyl)phenyl)piperidine-3- |
| | | | carboxamide |
| 177 | (2R,3S)-2-(4-(cyclopentyl(quinazolin-4- | 178 | (2R,3S)-2-(4-(cyclopentyl(pyrido[3,4- |
| | yl)amino)phenyl)-N-(4-methyl-3- | | d]pyrimidin-4-yl)amino)phenyl)-N-(4- |
| | (trifluoromethyl)phenyl)piperidine-3- | | methyl-3- |
| | carboxamide | | (trifluoromethyl)phenyl)piperidine-3- |
| | | | carboxamide |
| 179 | (2R,3S)-2-(4-(cyclopentyl(phthalazin-1- | 180 | ((2R,3S)-3-(6-(tert-butyl)-1H- |
| | yl)amino)phenyl)-N-(4-methyl-3- | | benzo[d]imidazol-2-yl)-2-(4- |
| | (trifluoromethyl)phenyl)piperidine-3- | | (cyclopentylamino)phenyl)piperidin-1- |
| | carboxamide | | yl)(2-fluoro-6-methylphenyl)methanone |
| 181 | ((2R,3S)-3-(5-(tert- | 182 | ((2R,3S,4aR,7aR)-2-(4- |
| | butyl)benzo[d]oxazol-2-yl)-2-(4- | | (cyclopentylamino)phenyl)-1-(2-fluoro-6- |
| | (cyclopentylamino)phenyl)piperidin-1- | | methylbenzoyl)octahydro-1H- |
| | yl)(2-fluoro-6-methylphenyl)methanone | | cyclopenta[b]pyridin-3-yl)(6-methyl-3,4- |
| | | | dihydroisoquinolin-2(1H)-yl)methanone |
| 183 | (2R,3S,4aR,7aR)-2-(4- | 184 | cis-2-(4-(cyclopentylamino)phenyl)-1-(2- |
| | (cyclopentyl(methyl)amino)phenyl)-1- | | fluoro-6-methylbenzoyl)-N-(1,2,3,4- |
| | (2-fluoro-6-methylbenzoyl)-N-(2- | | tetrahydroisoquinolin-6-yl)octahydro-1H- |
| | methyl-1,2,3,4-tetrahydroisoquinolin-6- | | cyclopenta[b]pyridine-3-carboxamide |
| | yl)octahydro-1H-cyclopenta[b]pyridine- | | |
| | 3-carboxamide | | |
| 185 | (2R,3S,4aR,7aR)-2-(4- | 186 | tert-butyl 6-((2R,3S,4aR,7aR)-2-(4- |
| | (cyclopentylamino)phenyl)-1-(2-fluoro- | | (cyclopentylamino)phenyl)-1-(2-fluoro-6- |
| | 6-methylbenzoyl)-N-(4-methyl-3- | | methylbenzoyl)octahydro-1H- |
| | (trifluoromethyl)benzyl)octahydro-1H- | | cyclopenta[b]pyridine-3-carboxamido)- |
| | cyclopenta[b]pyridine-3-carboxamide | | 3,4-dihydroisoquinoline-2(1H)- |
| | | | carboxylate |
| 187 | cis-3-(4-(cyclopentylamino)phenyl)-4- | 188 | (2R,3S)-2-(4-(N-cyclopentyl-2-fluoro-6- |
| | (2-fluoro-6-methylbenzoyl)-N-(4- | | methylbenzamido)phenyl)-N-(4-methyl-3- |
| | methyl-3- | | (trifluoromethyl)phenyl)piperidine-3- |
| | (trifluoromethyl)phenyl)morpholine-2- | | carboxamide |
| | carboxamide | | |
| 188 | (2R,3S)-2-(4- | 189 | cis-2-(4-(cyclopentylamino)phenyl)-1-(2- |
| | (cyclopentylamino)phenyl)-N-(4- | | fluoro-6-methylbenzoyl)-N-(1-methyl- |
| | methyl-3-(trifluoromethyl)phenyl)-1- | | 1H-pyrazolo[4,3-b]pyridin-6- |
| | (quinoline-8-carbonyl)piperidine-3- | | yl)octahydrofuro[3,4-b]pyridine-3- |
| | carboxamide | | carboxamide |
| 190 | (2R,3S)-2-(4- | 200 | (2R,3S)-2-(4-(cyclopentylamino)phenyl)- |
| | (cyclopentylamino)phenyl)-1-(3,5- | | N-(4-methyl-3- |
| | dimethylisoxazole-4-carbonyl)-N-(4- | | (trifluoromethyl)phenyl)piperidine-3- |
| | methyl-3- | | carboxamide |
| | (trifluoromethyl)phenyl)piperidine-3- | | |
| | carboxamide | | |
| 201 | cis-2-(4- | 202 | tert-butyl 6-cis-2-(4- |
| | (cyclopentyl(methyl)amino)phenyl)-1- | | (cyclopentylamino)phenyl)-1-(2-fluoro-6- |
| | (2-fluoro-6-methylbenzoyl)-N-(2- | | methylbenzoyl)octahydro-1H- |
| | methyl-1,2,3,4-tetrahydroisoquinolin-6- | | cyclopenta[b]pyridine-3-carboxamido)- |
| | yl)octahydro-1H-cyclopenta[b]pyridine- | | 3,4-dihydroisoquinoline-2(1H)- |
| | 3-carboxamide | | carboxylate |
| 203 | cis-1-(2-fluoro-6-methylbenzoyl)-N-(4- | 204 | cis-2-(4-(cyclopentylamino)phenyl)-1-(2- |
| | methyl-3-(trifluoromethyl)phenyl)-2-(2- | | fluoro-6-methylbenzoyl)-5-hydroxy-N-(4- |
| | oxaspiro[4.5]decan-8-yl)piperidine-3- | | methyl-3- |
| | carboxamide | | (trifluoromethyl)phenyl)piperidine-3- |
| | | | carboxamide |
| 205 | cis-1-(2-fluoro-6-methylbenzoyl)-N-(1- | 206 | cis-2-(4-(cyclopentylamino)phenyl)-1-(2- |
| | methyl-1H-indazol-5-yl)-2-(4-(((R)-2- | | fluoro-6-methylbenzoyl)-N-(4-methyl-3- |
| | (trifluoromethyl)pyrrolidin-1- | | (trifluoromethyl)phenyl)-6-(2,2,2- |
| | yl)methyl)phenyl)octahydro-1H- | | trifluoroethyl)octahydro-1H-pyrrolo[3,4- |
| | cyclopenta[b]pyridine-3-carboxamide | | b]pyridine-3-carboxamide |

Also provided are salts of compounds disclosed herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds described herein include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds described herein can generally be prepared by standard methods and techniques known to those skilled in the art or by procedures similar to those described in the accompanying Examples substituting appropriate isotopically-labeled reagents in place of the corresponding non-labeled reagent.

Solvates of a compound provided herein or a salt thereof are also contemplated. Solvates contain either stoichiometric or non-stoichiometric amounts of a solvent and are often formed during the process of crystallization. Hydrates are formed when the solvent is water, or alcoholates are formed when the solvent is alcohol.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

### Compositions

In another aspect, provided herein is a pharmaceutical composition comprising a compound disclosed herein, or a stereoisomer, tautomer, or a pharmaceutically acceptable salt of any of the foregoing, and a pharmaceutically acceptable carrier or excipient. In some embodiments, the pharmaceutical composition is administered in any suitable form and by any suitable route, such as by enteral administration (e.g., oral administration, sublingual administration, or rectal administration) or parenteral administration (e.g., intravenous injection, intramuscular injection, subcutaneous injection, intravenous infusion, or inhalation/insufflation).

In certain embodiments, pharmaceutical compositions are formulated in any manner, including using one or more physiologically acceptable carriers comprising excipients and/or auxiliaries which facilitate processing of the active compounds into pharmaceutical compositions. In some embodiments, proper formulation is dependent upon the route of administration chosen. In various embodiments, any techniques, carriers and excipients are used as suitable.

In some embodiments, a compound or composition disclosed herein is administered by enteral administration. Exemplary routes of enteral administration include, without limitation, oral administration, sublingual administration, and rectal administration (e.g., through the rectum). In some embodiments, the enteral administration comprises oral administration. In some embodiments, the enteral administration comprises sublingual administration. In some embodiments, the enteral administration comprises rectal administration.

In some embodiments, a compound or composition disclosed herein is administered by parenteral administration. Exemplary routes of parenteral administration include, without limitation, intravenous injection, intramuscular injection, subcutaneous injection, intravenous infusion, and inhalation/insufflation. In some embodiments, the parenteral administration comprises intravenous injection. In some embodiments, the parenteral administration comprises intramuscular injection. In some embodiments, the parenteral administration comprises subcutaneous injection. In some embodiments, the parenteral administration comprises intravenous infusion. In some embodiments, the parenteral administration comprises inhalation/insufflation.

In some embodiments, a compound or composition disclosed herein is administered by inhalation or insufflation. Exemplary types of preparations for inhalation and/or insufflation include, without limitation, sprays, aerosols, mists, capsules, powders, or cartridges for use in an inhaler or insufflator and solutions/suspensions for nebulization.

### Methods of Use

In another aspect, provided is a method of inhibit the binding of C5a receptor ligand (e.g., C5a) to C5a receptor in vitro or in vivo, the method comprising contacting a C5a receptor with an effective amount of the compound or composition disclosed herein. In some embodiments, the binding of C5a receptor ligand (e.g., C5a) to C5a receptor is inhibited by at least about 99%, at least about 98%, at least about 97%, at least about 96%, at least about 95%, at least about 90%, at least about 80%, at least about 70%, at least about 60%, at least about 50%, at least about 40%, at least about 30%, or at least about 20%. In some embodiments, provided is a method of inhibit the binding receptor in vitro or in vivo, the method comprising contacting a C5a receptor with an effective amount of the compound or composition disclosed herein.

The compound or salt thereof described herein can be used in combination with other treatment modalities, such as anti-inflammatory therapies. Examples of anti-inflammatory therapies that can be used in combination with the methods of the invention include, for example, therapies that employ steroidal drugs, as well as therapies that employ non-steroidal drugs.

In another aspect, provided is a method of treating a disorder mediated by C5a in a subject in need thereof, the method comprising administering a therapeutically effective amount of a compound or composition disclosed herein to the subject. In some embodiments, the disorder is an inflammatory disease, a cardiovascular or cerebrovascular disease, or an autoimmune disease.

In some embodiments, the disorder is an autoimmune disorder. Examples of autoimmune disorders include, but are not limited to, Rheumatoid arthritis, systemic lupus erythematosus, Guillain-Barre syndrome, pancreatitis, lupus nephritis, lupus glomerulonephritis, psoriasis, Crohn's disease, vasculitis, irritable bowel syndrome, dermatomyositis, multiple sclerosis, bronchial asthma, pemphigus, pemphigoid, scleroderma, myasthenia gravis, autoimmune hemolytic and thrombocytopenic states, Goodpasture's syndrome (and associated glomerulonephritis and pulmonary hemorrhage), immunovasculitis, tissue graft rejection, and hyperacute rejection of transplanted organs.

In some embodiments, the disorder is an inflammatory disorder or a related condition. Examples of inflammatory disorders and related conditions include, but are not limited to, Neutropenia, sepsis, septic shock, Alzheimer's disease, multiple sclerosis, stroke, inflammatory bowel disease (IBD), inflammation associated with severe burns, lung injury, and ischemia-reperfusion injury, osteoarthritis, as well as acute (adult) respiratory distress syndrome (ARDS), chronic pulmonary obstructive disorder (COPD), systemic inflammatory response syndrome (SIRS), atopic dermatitis, psoriasis, chronic urticaria and multiple organ dysfunction syndrome (MODS). Also included are pathologic sequellae associated with insulin-dependent diabetes mellitus (including diabetic retinopathy), lupus nephropathy, Heyman nephritis, membranous nephritis and other forms of glomerulonephritis, contact sensitivity responses, and inflammation resulting from contact of blood with artificial surfaces that can cause complement activation, as occurs, for example, during extracorporeal circulation of blood (e.g., during hemodialysis or via a heart-lung machine, for example, in association with vascular surgery such as coronary artery bypass grafting or heart valve replacement), or in association with contact with other artificial vessel or container surfaces (e.g., ventricular assist devices, artificial heart machines, transfusion tubing, blood storage bags, plasmapheresis, plateletpheresis, and the like).

In some embodiments, the disorder is a disorder related to ischemia/reperfusion injury. Examples of disorders related to ischemia/reperfusion injury include, but are not limited to, those resulting from transplants, including solid organ transplant, and syndromes such as ischemic reperfusion injury, ischemic colitis and cardiac ischemia.

In some embodiments, the disorder is age-related macular degeneration.

In some embodiments, the disorder is a cardiovascular or cerebrovascular disorder. Examples of cardiovascular or cerebrovascular disorders include, but are not limited to, myocardial infarction, coronary thrombosis, vascular occlusion, post-surgical vascular reocclusion, atherosclerosis, traumatic central nervous system injury, and ischemic heart disease. In one embodiment, an effective amount of a compound of the invention may be administered to a patient at risk for myocardial infarction or thrombosis (i.e., a patient who has one or more recognized risk factor for myocardial infarction or thrombosis, such as, but not limited to, obesity, smoking, high blood pressure, hypercholesterolemia, previous or genetic history of myocardial infarction or thrombosis) in order reduce the risk of myocardial infarction or thrombosis.

In some embodiments, the disorder is a vasculitic disease. Examples of vasculitic diseases include, but are not limited to, Wegener's granulomatosis, microscopic polyangiitis, Churg-Strauss syndrome, Henoch-Schonlein purpura, polyarteritis nodosa, Rapidly Progressive Glomerulonephritis (RPGN), cryoglobulinaemia, giant cell arteritis (GCA), Behcet's disease and Takayasu's arteritis (TAK).

In some embodiments, the disorder is selected from: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD), bullous pemphigoid, hidradenitis suppurativa, dermatitis herpetiformis, sweets syndrome, pyoderma gangrenosum, palmo-plantar pustulosis & pustular psoriasis, rheumatoid neutrophilic dermatoses, subcorneal pustular dermatosis, bowel-associated dermatosis-arthritis syndrome, neutrophilic eccrine hidradenitis, linear IgA disease, or any combinations thereof.

In some embodiments, the disorder is HIV infection or AIDS.

In some embodiments, the compounds or salts thereof reduce neutropenia induced by human C5a in a subject. In some embodiments, the compounds or salts thereof reduce neutropenia induced by human C5a in a subject by reducing at least 10%, 20%, 30%, 40%, 50%, 60%, 70%, 80%, or 90% of the neutrophil cell counts. In some embodiments, the subject is a human C5aR knock-in mice. In some embodiments, the subject is a cyno monkey. In some embodiments, the subject is a human. In some embodiments, the human C5a induced neutropenia is induced by intravitreal injection of human C5a. In some embodiments, the human C5a induced neutropenia is induced by oral dosing of human C5a.

In some embodiments, the compounds or salts thereof block human C5a induced CD11b upregulation on immune cells. In some embodiments, the immune cell is a granulocyte. In some embodiments, the immune cell is a neutrophil. In some embodiments, the compounds or salts thereof reduce CD11b upregulation induced by human C5a in a subject by reducing at least 10%, 20%, 30%, 40%, 50%, 60%, 70%, 80%, or 90% of the CD11b expressed by granulocytes. In some embodiments, the compounds or salts thereof reduce CD11b upregulation induced by human C5a in a subject by reducing at least 10%, 20%, 30%, 40%, 50%, 60%, 70%, 80%, or 90% of the CD11b expressed by neutrophils. In some embodiments, the subject is a human C5aR knock-in mice. In some embodiments, the subject is a cyno monkey. In some embodiments, the subject is a human. In some embodiments, the human C5a induced upregulation of CD11b is induced by intravitreal injection of human C5a. In some embodiments, the human C5a induced CD11b upregulation is induced by oral dosing of human C5a.

### Dosing

Dosages and desired drug concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

Typically, dosages which may be administered in a method of the invention to a subject, in some embodiments a human, range in amount from 0.5 ng to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

A compound or composition disclosed herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). In some embodiments of a method disclosed herein, a compound or composition disclosed herein is administered four times a day, three time a day, twice a day, or once a day.

### Articles of Manufacture and Kits

In another aspect, provided is an article of manufacture comprising a compound described herein or a composition described herein in suitable packaging. In some embodiments, the article of manufacture is for use in any of the methods described herein. Suitable packaging is known in the art and includes, for example, vials, vessels, ampules, bottles, jars, flexible packaging and the like An article of manufacture may further be sterilized and/or sealed.

In another aspect, provided is a kit comprising a compound described herein or a composition described herein. The kits may be used for any one or more of the uses described herein, and, accordingly, may contain instructions for the treatment of any disorder disclosed herein. Each component (if there is more than one component) can be packaged in separate containers or some components can be combined in one container where cross-reactivity and shelf life permit.

### EXAMPLES

It is understood that the present disclosure has been made only by way of example, and that numerous changes in the combination and arrangement of parts can be resorted to by those skilled in the art without departing from the spirit and scope of the present disclosure.

Compounds of formula (I) or any sub-formula described herein can be synthesized using standard synthetic techniques known to those of skill in the art. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or be making routine modifications of reaction conditions, reagents, and starting materials. Alternatively, other reactions disclosed herein or known in the art will be recognized as having applicability for preparing other compounds of the present disclosure.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

### Synthetic Examples

### Example S1: Synthesis of (2R,3S)-2-(4-(cyclopentylamino)phenyl)-N-(4-methyl-3-(trifluoro-methyl)phenyl)-1-(thieno[2,3-c]pyridin-7-yl)piperidine-3-carboxamide (Compound No. 1)

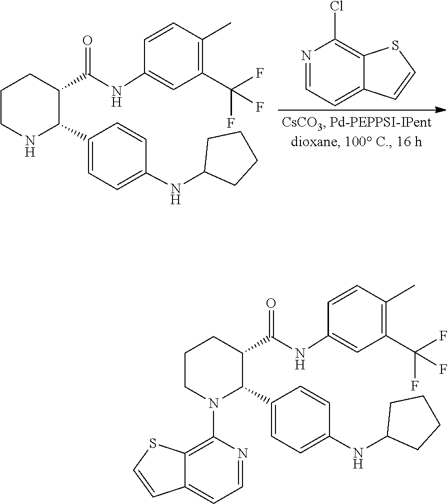 [SMILES: Cc1ccc(NC(=O)[C@H]2CCCN[C@H]2c2ccc(NC3CCCC3)cc2)cc1C(F)(F)F ; Clc1nccc2ccsc12 ; Cc1ccc(NC(=O)[C@H]2CCCN(c3nccc4ccsc34)[C@H]2c2ccc(NC3CCCC3)cc2)cc1C(F)(F)F]

To a mixture of (2R,3S)-2-[4-(cyclopentylamino)phenyl]-N-[4-methyl-3-(trifluoro methyl)phenyl]piperidine-3-carboxamide (50 mg, 95.39 mol), 7-chlorothieno [2,3-c]pyridine (32.36 mg, 190.79 mol) and Cs₂CO₃ (93.24 mg, 286.18 mol) in dioxane (1 mL) was added Pd-PEPPSI-IPent (7.57 mg, 9.54 mol). The mixture was charged with N₂, and then stirred at 100° C. for 16 h. The mixture combined with previous batches (38.16 mol and 95.39 mol) was filtered through a pad of Celite and rinsed with EtOAc (20 mL). The filtrate was concentrated to give the crude product (250 mg) as brown oil. The crude product was purified by flash silica gel chromatography (ISCO®; 4 g SepaFlash® Silica Flash Column, Eluent of 0-10% Dichloromethane: Methanol gradient@25 mL/min) to give a crude product (70 mg), which was further purified by prep-HPLC (column: Xtimate C18 10 μm 250 mm×50 mm; mobile phase: [water (0.04% NH₃H₂O+10 mM NH₄HCO₃)-ACN]; B %: 70%-100%, 8 min) to give the crude. The crude product was purified again by prep-TLC (Dichloromethane:Methanol=10:1) to give (2R,3S)-2-[4-(cyclopentylamino)phenyl]-N-[4-methyl-3-(trifluoromethyl)phenyl]-1-thieno[2,3-c]pyridin-7-yl-piperidine-3-carboxamide (5 mg, 7.95 mol, 8.33% yield, 92% purity) as an off-white solid. ¹H NMR (400 MHz, CDCl₃) δ 1.23-1.48 (m, 6H), 1.66-1.77 (m, 6H), 1.87-2.04 (m, 4H), 2.30-2.47 (m, 6 H), 3.27 (br d, J=4.0 Hz, 1H), 3.58-3.76 (m, 3H), 3.98-4.05 (m, 1H), 6.07 (br s, 1H), 6.49 (d, J=8.5 Hz, 2H), 7.10 (d, J=8.5 Hz, 2H), 7.20 (d, J=8.5 Hz, 1H), 7.25 (s, 1H), 7.30-7.41 (m, 2H), 7.66 (d, J=5.5 Hz, 2H), 7.80 (br d, J=7.8 Hz, 1H), 8.13 (d, J=5.5 Hz, 1H), 10.14 (br s, 1H). LC-MS: (ES) m/z 579.2 (M+H⁺).

### Example S2: Synthesis of (2R,3S)-1-(2-chloropyrimidin-4-yl)-2-(4-(cyclopentylamino)phenyl)-N-(4-methyl-3-(trifluoromethyl)phenyl)piperidine-3-carboxamide (Compound No. 2)

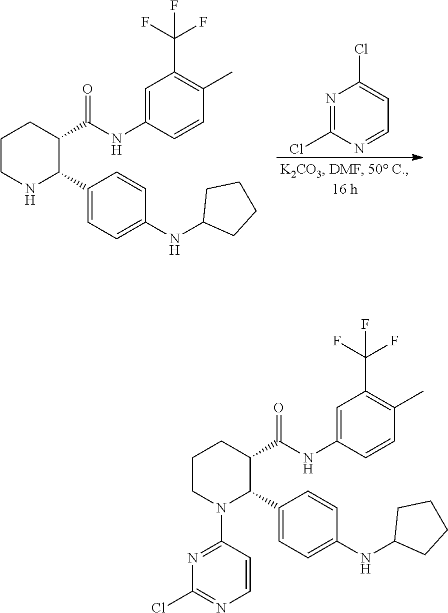 [SMILES: Cc1ccc(NC(=O)[C@H]2CCCN[C@H]2c2ccc(NC3CCCC3)cc2)cc1C(F)(F)F ; Clc1ccnc(Cl)n1 ; Cc1ccc(NC(=O)[C@H]2CCCN(c3ccnc(Cl)n3)[C@H]2c2ccc(NC3CCCC3)cc2)cc1C(F)(F)F]

A mixture of (2R,3S)-2-[4-(cyclopentylamino) phenyl]-N-[4-methyl-3-(trifluoromethyl) phenyl]piperidine-3-carboxamide (300 mg, 572.37 mol), 2,4-dichloropyrimidine (170.54 mg, 1.14 mmol) and K₂CO₃ (237.32 mg, 1.72 mmol) in DMF (3 mL) was stirred at 50° C. for 16 h. The combined organic layers were washed with brine (15 mL×4), dried over anhydrous Na₂SO₄, filtered and concentrated to give the crude product (450 mg) as a brown gum. The crude product was diluted with MeCN (2.5 mL), filtered, and sent to be purified by prep-HPLC (column: Xtimate C18 10 μm 250 mm×50 mm; mobile phase: [water (0.04% NH₃H₂O+10 mM NH₄HCO₃)-ACN]; B %: 70%-100%, 8 min). Compound (2R,3S)-1-(2-chloropyrimidin-4-yl)-2-[4-(cyclopentylamino)phenyl]-N-[4-methyl-3-(trifluoromethyl)phenyl] piperidine-3-carboxamide (150 mg, 255.36 mol, 44.61% yield, 95% purity) was obtained as an off-white solid. ¹H NMR (400 MHz, CDCl₃) δ 1.35-1.47 (m, 2H), 1.53-1.75 (m, 8H), 1.91-2.34 (m, 6H), 2.42 (d, J=1.5 Hz, 3H), 2.97-3.08 (m, 1H), 3.21-3.34 (m, 1H), 3.71 (quin, J=6.1 Hz, 1H), 4.08 (br s, 1H), 6.08 (br s, 1H), 6.39-6.53 (m, 3H), 7.09-7.23 (m, 3H), 7.51 (dd, J=8.3, 2.0 Hz, 1H), 7.58 (s, 1H), 7.88 (br s, 1H), 8.03 (d, J=6.0 Hz, 1H). LC-MS: (ES) m/z 558.2 (M+H⁺).

### Example S3: Synthesis of (2R,3R)-2-(4-(cyclopentylamino)phenyl)-N-(4-methyl-3-(trifluoromethyl)phenyl)-1-(pyrimidin-4-yl)piperidine-3-carboxamide and (2R,3S)-2-(4-(cyclopentylamino)phenyl)-N-(4-methyl-3-(trifluoromethyl)phenyl)-1-(pyrimidin-4-yl)piperidine-3-carboxamide (Compound Nos. 3 and 4)

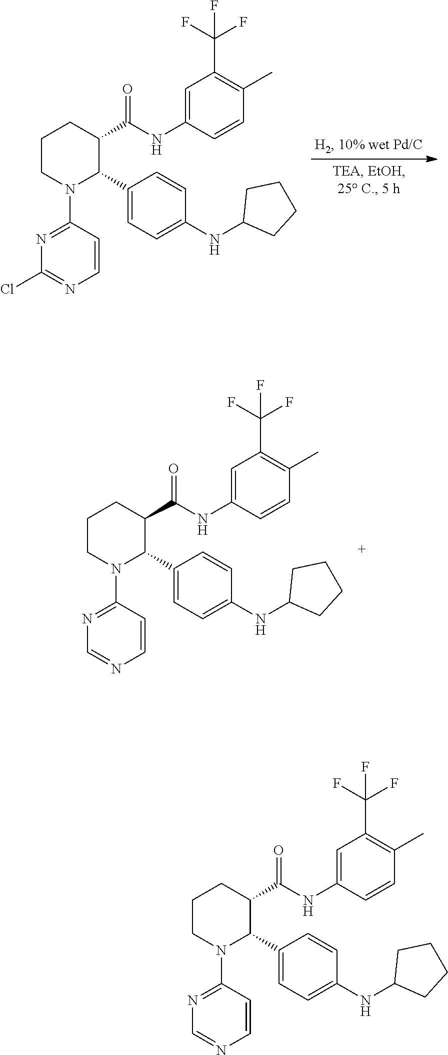 [SMILES: Cc1ccc(NC(=O)[C@H]2CCCN(c3ccnc(Cl)n3)[C@H]2c2ccc(NC3CCCC3)cc2)cc1C(F)(F)F ; Cc1ccc(NC(=O)[C@@H]2CCCN(c3ccncn3)[C@H]2c2ccc(NC3CCCC3)cc2)cc1C(F)(F)F ; Cc1ccc(NC(=O)[C@H]2CCCN(c3ccncn3)[C@H]2c2ccc(NC3CCCC3)cc2)cc1C(F)(F)F]

To a mixture of (2R,3S)-1-(2-chloropyrimidin-4-yl)-2-[4-(cyclopentylamino)phenyl]-N-[4-methyl-3-(trifluoromethyl)phenyl]piperidine-3-carboxamide (150 mg, 268.80 mol) and TEA (27.20 mg, 268.80 mol, 37.41 μL) in EtOH (15 mL) was added Pd/C (10% wet basis) (60 mg, 10% purity) under Ar atmosphere. The suspension was degassed and purged with H₂ for 3 times. The mixture was filtered through a pad of celite, and the filtrate was concentrated to give the crude product (150 mg) as a gray gum. The crude product combined with the previous batch (50 mg, 89.6 mol) was diluted with MeOH (3 mL), filtered, and sent to be purified by prep-HPLC. The product (100 mg) as a white solid was obtained from purification of prep-HPLC (column: Xtimate C18 10μ250 mm×50 mm; mobile phase: [water (0.04% NH₃H₂O+10 mM NH₄HCO₃)-ACN]; B %: 65%-95%, 8 min). Then the solid was further purified by flash silica gel chromatography (ISCO®; 12 g SepaFlash® Silica Flash Column, Eluent of 0˜100% Ethyl acetate/Petroleum ether gradient@ 30 mL/min) to give two compounds.

(2R,3R)-2-[4-(cyclopentylamino) phenyl]-N-[4-methyl-3-(trifluoromethyl) phenyl]-1-pyrimidin-4-yl-piperidine-3-carboxamide (5 mg, 9.55 mol, 3.55% yield, 100% purity) was isolated as a gray solid. ¹H NMR (400 MHz, CDCl₃) δ 0.79-1.06 (m, 1H), 0.79-1.06 (m, 1 H), 0.79-1.06 (m, 2H), 1.18-1.35 (m, 3H), 1.39-1.50 (m, 2H), 1.64-1.82 (m, 6H), 1.94-2.08 (m, 3H), 2.32 (br dd, J=13.8, 4.3 Hz, 1H), 2.42 (s, 3H), 3.21 (td, J=12.7, 4.3 Hz, 1 H), 3.33 (br d, J=3.5 Hz, 1H), 3.71-3.81 (m, 1H), 4.05 (br d, J=9.8 Hz, 1H), 6.35 (br s, 1 H), 6.53-6.63 (m, 3H), 6.95 (d, J=8.5 Hz, 2H), 7.20 (d, J=8.3 Hz, 1H), 7.55 (br d, J=8.3 Hz, 1H), 7.72 (s, 1H), 8.26 (d, J=6.3 Hz, 1H), 8.66-8.77 (m, 2H). LC-MS: (ES) m/z 524.3 (M+H⁺).

(2R,3S)-2-[4-(cyclopentylamino)phenyl]-N-[4-methyl-3-(trifluoromethyl) phenyl]-1-pyrimidin-4-yl-piperidine-3-carboxamide (40 mg, 76.40 mol, 28.42% yield, 100% purity) was obtained as a white solid. ¹H NMR (400 MHz, CDCl₃) δ 1.43 (br d, J=4.8 Hz, 2H), 1.54-1.73 (m, 6H), 1.92-2.02 (m, 3H), 2.11 (br d, J=10.0 Hz, 1H), 2.26 (qd, J=13.0, 4.5 Hz, 1 H), 2.42 (d, J=1.3 Hz, 3H), 3.00 (dt, J=12.9, 4.5 Hz, 1H), 3.22 (td, J=13.4, 3.4 Hz, 1H), 3.71 (quin, J=6.1 Hz, 1H), 4.01 (br d, J=13.6 Hz, 1H), 6.27 (br s, 1H), 6.44-6.57 (m, 3H), 7.10-7.24 (m, 3H), 7.51-7.66 (m, 2H), 8.03 (br s, 1H), 8.19 (d, J=6.3 Hz, 1H), 8.64 (s, 1 H). LC-MS: (ES) m/z 524.3 (M+H⁺).

### Example S4: Synthesis of benzyl cyclopentyl(4-((2R,3S)-3-((4-methyl-3-(trifluoromethyl)-phenyl)carbamoyl)-1-(quinazolin-4-yl)piperidin-2-yl)phenyl)carbamate (Compound No. 5)

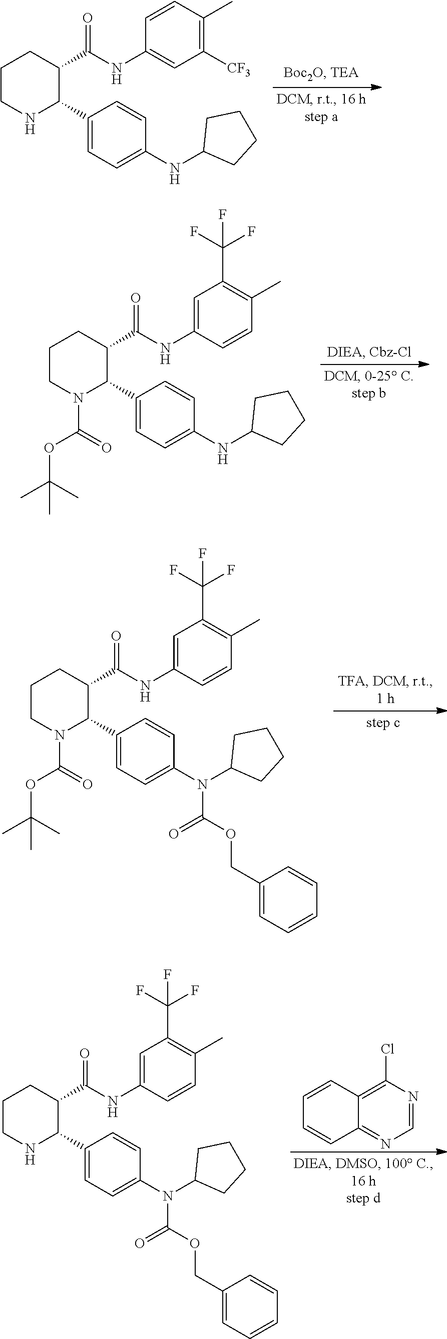 [SMILES: Cc1ccc(NC(=O)[C@H]2CCCN[C@H]2c2ccc(NC3CCCC3)cc2)cc1C(F)(F)F ; Cc1ccc(NC(=O)[C@H]2CCCN(C(=O)OC(C)(C)C)[C@H]2c2ccc(NC3CCCC3)cc2)cc1C(F)(F)F ; Cc1ccc(NC(=O)[C@H]2CCCN(C(=O)OC(C)(C)C)[C@H]2c2ccc(N(C(=O)OCc3ccccc3)C3CCCC3)cc2)cc1C(F)(F)F ; Cc1ccc(NC(=O)[C@H]2CCCN[C@H]2c2ccc(N(C(=O)OCc3ccccc3)C3CCCC3)cc2)cc1C(F)(F)F ; Clc1ncnc2ccccc12]

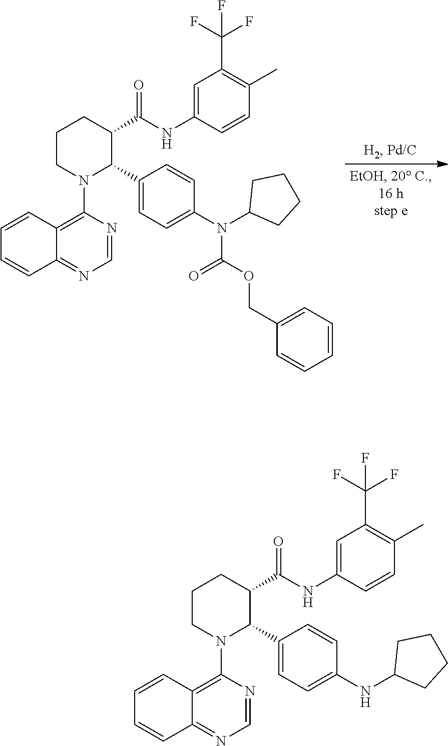 [SMILES: Cc1ccc(NC(=O)[C@H]2CCCN(c3ncnc4ccccc34)[C@H]2c2ccc(N(C(=O)OCc3ccccc3)C3CCCC3)cc2)cc1C(F)(F)F ; Cc1ccc(NC(=O)[C@H]2CCCN(c3ncnc4ccccc34)[C@H]2c2ccc(NC3CCCC3)cc2)cc1C(F)(F)F]

Step a) To a mixture of (2R,3S)-2-[4-(cyclopentylamino)phenyl]-N-[4-methyl-3-(trifluoro methyl)phenyl]piperidine-3-carboxamide (5 g, 9.54 mmol) and tert-butoxycarbonyl tert-butyl carbonate (2.08 g, 9.54 mmol, 2.19 mL) in DCM (50 mL) was added TEA (1.93 g, 19.08 mmol, 2.66 mL). The solution was stirred at 25° C. for 16 h. The reaction mixture was added to 30 mL of H₂O and extracted with DCM (50 mL×2). The combined organic layers were washed with brine (50 mL), dried with anhydrous Na₂SO₄ and filtered. The filtrate was evaporated under vacuum to give crude product. The crude product was purified by column chromatography (SiO₂, Petroleum ether/Ethyl acetate=100/0 to 3:1) to give tert-butyl (2R,3S)-2-[4-(cyclopentyl amino)phenyl]-3-[[4-methyl-3-(trifluoromethyl)phenyl]carbamoyl]piperidine-1-carboxylate (4.2 g, 7.70 mmol, 80.69% yield) as a pale yellow solid. ¹H NMR (400 MHz, DMSO-d₆) δ 1.22-1.52 (14H, m) 1.53-1.66 (2H, m) 1.67-1.87 (4H, m) 1.89-2.02 (1H, m) 2.32 (3H, br d, J=1.22 Hz) 2.77-3.00 (2H, m) 3.56 (1H, t, J=6.16 Hz) 3.83 (1H, brd, J=11.00 Hz) 5.28-5.76 (2H, m) 6.35 (2H, d, J=8.80 Hz) 6.96 (2H, br d, J=8.31 Hz) 7.28 (1H, d, J=8.31 Hz) 7.54 (1 H, br s) 7.81 (1 H, br s) 10.17 (1 H, br s). LC-MS: (ES) m/z 546.3 (M+H⁺).

Step b) To a mixture of tert-butyl (2R,3S)-2-[4-(cyclopentylamino)phenyl]-3-[[4-methyl-3-(trifluoromethyl)phenyl]carbamoyl]piperidine-1-carboxylate (2 g, 3.30 mmol) and DIEA (1.28 g, 9.90 mmol, 1.72 mL) in DCM (20 mL) was added Cb₂Cl (1.13 g, 6.60 mmol, 937.95 μL) at 0° C. Then the mixture was stirred at 25° C. for 12 h. The previous reaction mixture (100 mg batch) was combined with this batch. The combined mixture was quenched by addition of H₂O (20 mL) and extracted with DCM (30 mL). The organic phase separated was concentrated in vacuo to give the crude product. The crude product was purified by flash silica gel chromatography (ISCO®; 40 g SepaFlash® Silica Flash Column, Eluent of 0˜30% Ethyl acetate/Petroleum ether gradient@ 40 mL/min). The desired compound tert-butyl (2R,3S)-2-[4-[benzyloxycarbonyl (cyclopentyl)amino]phenyl]-3-[[4-methyl-3-(trifluoromethyl)phenyl]carbamoyl]piperidine-1-carboxylate (1.9 g, 100% purity) was obtained as white solid. ¹H NMR (400 MHz, METHANOL-d₄) δ 1.27-1.55 (m, 15H), 1.56-1.72 (m, 1H), 1.75-2.01 (m, 4H), 2.05-2.21 (m, 1H), 2.38 (d, J=1.51 Hz, 3H), 3.06 (ddd, J=12.99, 6.21, 3.89 Hz, 1H), 3.18 (br s, 1H), 4.04 (br dd, J=13.68, 3.39 Hz, 1H), 4.40-4.54 (m, 1H), 4.99-5.06 (m, 2H), 5.70 (br s, 1H), 7.05 (d, J=8.28 Hz, 2H), 7.14 (br s, 2H), 7.18-7.29 (m, 4H), 7.39 (br d, J=8.28 Hz, 2H), 7.46 (br d, J=7.78 Hz, 1H), 7.75 (d, J=1.51 Hz, 1H). LC-MS: (ES) m/z 680.3 (M+H⁺).

Step c) To a mixture of tert-butyl (2R,3S)-2-[4-[benzyloxycarbonyl(cyclopentyl)amino]phenyl]-3-[[4-methyl-3-(trifluoromethyl)phenyl]carbamoyl]piperidine-1-carboxylate (0.3 g, 441.33 mol) in DCM (3 mL) was added TFA (1.54 g, 13.51 mmol, 1 mL). Then the mixture was stirred at 25° C. for 1 h. The reaction mixture was diluted with DCM (10 mL) and quenched by addition of saturated Na₂CO₃ solution to pH=9˜10. The organic phase separated was dried, filtered and concentrated in vacuo to give the desired product benzyl N-cyclopentyl-N-[4-[(2R,3S)-3-[[4-methyl-3-(trifluoromethyl)phenyl]carbamoyl]-2-piperidyl]phenyl]carbamate (0.24 g, 401.62 mol, 91.00% yield, 97% purity) as off-white solid. ¹H NMR (400 MHz, METHANOL-d₄) δ 1.19-1.43 (m, 7H), 1.71-1.82 (m, 2H), 1.99-2.20 (m, 3H), 2.36 (d, J=1.22 Hz, 3H), 2.79-2.95 (m, 2H), 3.36 (br d, J=11.49 Hz, 1H), 4.08 (d, J=3.42 Hz, 1H), 4.45 (quin, J=8.07 Hz, 1H), 4.97 (s, 2H), 7.07 (br d, J=8.31 Hz, 4H), 7.15-7.26 (m, 4H), 7.39 (d, J=8.31 Hz, 3H), 7.72 (d, J=1.96 Hz, 1H). LC-MS: (ES) m/z 580.3 (M+H⁺).

Step d) A mixture of benzyl N-cyclopentyl-N-[4-[(2R,3S)-3-[[4-methyl-3-(trifluoromethyl) phenyl]carbamoyl]-2-piperidyl]phenyl]carbamate (150 mg, 258.78 mol), 4-chloro quinazoline (60 mg, 364.54 μmol) and DIEA (100.33 mg, 776.33 mol, 135.22 μL) in DMSO (0.5 mL) was stirred at 100° C. for 16 hr. The reaction mixture was diluted with H₂O (20 mL) and extracted with EtOAc 60 mL (30 mL×2). The combined organic layers were dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 35 mL/min) to give the crude benzyl N-cyclopentyl-N-[4-[(2R,3S)-3-[[4-methyl-3-(trifluoromethyl)phenyl]carbamoyl]-1-quinazolin-4-yl-2-piperidyl]phenyl]carbamate (94 mg, 99.61 mol, 38.49% yield, 75% purity) as light yellow solid. The crude was further purified by prep-HPLC (basic condition) column: Xtimate C18 10 μm 250 mm×50 mm; mobile phase: [water (0.04% NH₃H₂O+10 mM NH₄HCO₃)-ACN]; B %: 80%-100%, 8 min) to give compound benzylN-cyclopentyl-N-[4-[(2R,3S)-3-[[4-methyl-3-(trifluoromethyl) phenyl]carbamoyl]-1-quinazolin-4-yl-2-piperidyl]phenyl] carbamate (77 mg, 105.53 mol, 79.46% yield, 97% purity) as a white solid. ¹H NMR (400 MHz, CDCl₃) δ 1.33-1.44 (m, 2H), 1.49 (br s, 4H), 1.82-1.96 (m, 3H), 2.14-2.24 (m, 1H), 2.30-2.41 (m, 1H), 2.44 (d, J=1.25 Hz, 3H), 3.40-3.53 (m, 2H), 4.08 (br d, J=13.05 Hz, 1H), 4.44-4.57 (m, 1H), 5.09 (s, 2H), 6.30 (br d, J=4.02 Hz, 1H), 7.09 (d, J=8.53 Hz, 2H), 7.16 (br s, 2H), 7.20-7.26 (m, 4H), 7.46-7.58 (m, 3H), 7.64 (br d, J=8.28 Hz, 1H), 7.76-7.84 (m, 2H), 7.94 (dd, J=8.28, 5.02 Hz, 2H), 8.41 (br s, 1H), 8.78 (s, 1H). LC-MS: (ES) m/z 708.3 (M+H⁺).

Step e) A mixture of benzyl N-cyclopentyl-N-[4-[(2R,3S)-3-[[4-methyl-3-(trifluoromethyl) phenyl] carbamoyl]-1-quinazolin-4-yl-2-piperidyl]phenyl]carbamate (50 mg, 70.64 mol) and Pd/C (wet) (20 mg, 10% purity) in EtOH (20 mL) was degassed and purged with H₂ (15 psi) 3 times. Then the mixture was stirred at 20° C. for 16 h under H₂ atmosphere. The reaction mixture was filtered and concentrated under reduced pressure to give a residue. The residue was purified by prep-HPLC (basic condition, column: Xtimate C18 10 μm 250 mm×50 mm; mobile phase: [water (0.04% NH₃·H₂O+10 mM NH₄HCO₃)-ACN]; B %: 70%-100%, 8 min) to give (2R,3S)-2-[4-(cyclopentylamino)phenyl]-N-[4-methyl-3-(trifluoromethyl)phenyl]-1-quinazolin-4-yl-piperidine-3-carboxamide (7 mg, 11.84 mol, 16.76% yield, 97% purity) as a white solid. ¹H NMR (400 MHz, CDCl₃) δ 1.40-1.50 (m, 2H), 1.58-1.66 (m, 2H), 1.68-1.76 (m, 2H), 1.87 (br d, J=13.21 Hz, 1H), 1.96-2.01 (m, 2H), 2.10-2.18 (m, 1H), 2.30-2.39 (m, 1H), 2.43 (s, 3H), 3.32 (dt, J=12.04, 4.00 Hz, 1H), 3.47-3.60 (m, 1H), 3.71-3.79 (m, 1H), 4.09 (br d, J=13.21 Hz, 1H), 6.23 (br d, J=4.16 Hz, 1H), 6.54 (d, J=8.80 Hz, 2H), 7.20 (d, J=8.31 Hz, 1H), 7.30 (d, J=8.56 Hz, 2 H), 7.43 (t, J=7.58 Hz, 1H), 7.63 (br d, J=8.31 Hz, 1H), 7.73 (t, J=7.70 Hz, 1H), 7.77 (s, 1 H), 7.87 (d, J=8.80 Hz, 2H), 8.70 (s, 1H), 8.78 (br s, 1H). LC-MS: (ES) m/z 574.3 (M+H⁺).

### Example S5: Synthesis of benzyl cyclopentyl(4-((2R,3S)-3-((4-methyl-3-(trifluoromethyl)phenyl)-carbamoyl)-1-(pyrido[3,2-d]pyrimidin-4-yl)piperidin-2-yl)phenyl)carbamate (Compound No. 166)

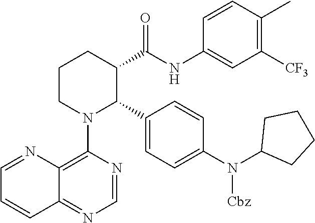 [SMILES: Cc1ccc(NC(=O)[C@H]2CCCN(c3ncnc4cccnc34)[C@H]2c2ccc(N(C(=O)OCc3ccccc3)C3CCCC3)cc2)cc1C(F)(F)F]

The title compound was synthesized in similar fashion as Example S4. ¹H NMR (400 MHz, CDCl₃) δ 1.31-1.43 (m, 2H), 1.50 (br d, J=4.02 Hz, 4H), 1.77-2.02 (m, 6H), 2.21 (br d, J=13.30 Hz, 1H), 2.36-2.54 (m, 4H), 3.11 (br s, 1H), 3.34 (br s, 1H), 4.43-4.58 (m, 1H), 5.03-5.13 (m, 2H), 7.07 (d, J=8.53 Hz, 2H), 7.13 (br s, 2H), 7.18-7.26 (m, 4H), 7.50 (br d, J=7.78 Hz, 2H), 7.61-7.75 (m, 2H), 7.81 (s, 1H), 8.19 (br d, J=8.03 Hz, 1 H), 8.44 (br s, 1H), 8.69 (s, 1H), 8.80 (br d, J=2.51 Hz, 1H). LC-MS: (ES) m/z 709.3 (M+H⁺).

### Example S6: Synthesis of (2R,3S)-2-(4-(cyclopentylamino)phenyl)-N-(4-methyl-3-(trifluoromethyl)phenyl)-1-(pyrido[3,2-d]pyrimidin-4-yl)piperidine-3-carboxamide (Compound No. 161)

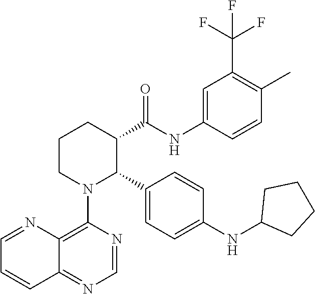 [SMILES: Cc1ccc(NC(=O)[C@H]2CCCN(c3ncnc4cccnc34)[C@H]2c2ccc(NC3CCCC3)cc2)cc1C(F)(F)F]

The title compound was synthesized in a similar fashion as the compound in S4: ¹H NMR (400 MHz, DMSO-d₆) δ 1.40 (dt, J=12.30, 6.15 Hz, 2H), 1.47-1.57 (m, 2H), 1.60-1.70 (m, 2H), 1.78-1.92 (m, 3H), 1.92-2.10 (m, 2H), 2.23-2.36 (m, 1H), 2.38 (s, 3H), 3.14-3.21 (m, 1H), 3.33 (br t, J=12.67 Hz, 1H), 3.58-3.70 (m, 1H), 5.12 (br d, J=6.53 Hz, 1H), 5.44 (br s, 1H), 6.45 (d, J=8.28 Hz, 2H), 7.22 (d, J=8.53 Hz, 2H), 7.32 (br d, J=8.53 Hz, 2H), 7.66 (br d, J=8.28 Hz, 1H), 7.77 (dd, J=8.53, 4.02 Hz, 1H), 7.89 (s, 1H), 8.12 (d, J=8.53 Hz, 1H), 8.58 (s, 1H), 8.80 (dd, J=4.14, 1.63 Hz, 1H), 9.74 (br s, 1H). LC-MS: (ES) m z 575.3 (M+H⁺).

### Example S7: Synthesis of (2R,3S)-2-(4-(cyclopentylamino)phenyl)-N-(4-methyl-3-(trifluoro-methyl)phenyl)-1-(1,7-naphthyridin-8-yl)piperidine-3-carboxamide (Compound No. 6)

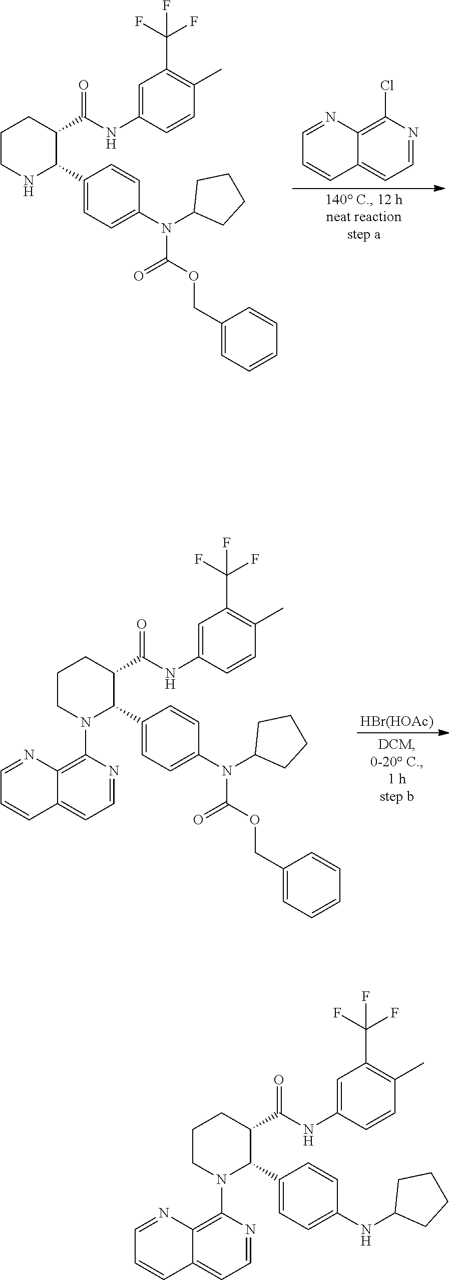 [SMILES: Cc1ccc(NC(=O)[C@H]2CCCN[C@H]2c2ccc(N(C(=O)OCc3ccccc3)C3CCCC3)cc2)cc1C(F)(F)F ; Clc1nccc2cccnc12 ; Cc1ccc(NC(=O)[C@H]2CCCN(c3nccc4cccnc34)[C@H]2c2ccc(N(C(=O)OCc3ccccc3)C3CCCC3)cc2)cc1C(F)(F)F ; "HBr(HOAc)" ; Cc1ccc(NC(=O)[C@H]2CCCN(c3nccc4cccnc34)[C@H]2c2ccc(NC3CCCC3)cc2)cc1C(F)(F)F]

Step a) A solution of benzyl N-cyclopentyl-N-[4-[(2R,3S)-3-[[4-methyl-3-(trifluoromethyl) phenyl]carbamoyl]-2-piperidyl]phenyl]carbamate (0.2 g, 345.04 mol) and 8-chloro-1,7-naphthyridine (0.1 g, 607.56 mol) in dioxane (1 mL) was concentrated in vacuo to give the residue. The residue was stirred at 140° C. for 12 h. The residue was purified by flash silica gel chromatography (ISCO®; 12 g SepaFlash® Silica Flash Column, Eluent of 0˜30% Ethyl acetate/Petroleum ether gradient@18 mL/min) to give benzyl N-cyclopentyl-N-[4-[(2R,3S)-3-[[4-methyl-3-(trifluoromethyl)phenyl]carbamoyl]-1-(1,7-naphthyridin-8-yl)-2-piperidyl]phenyl]carbamate (65 mg, 91.84 mol, 26.62% yield, 100% purity) as light yellow solid. ¹H NMR (400 MHz, METHANOL-d₄) δ1.24-1.32 (m, 2H), 1.40 (br d, J=4.40 Hz, 5H), 1.75 (br d, J=5.14 Hz, 2H), 1.90 (br dd, J=8.44, 4.28 Hz, 1H), 2.09-2.27 (m, 2H), 2.31-2.37 (m, 1H), 2.41 (s, 3H), 3.61-3.74 (m, 1H), 3.87 (br s, 1H), 4.32-4.46 (m, 1H), 4.96 (s, 2H), 6.09 (br s, 1H), 6.85 (d, J=8.31 Hz, 2H), 7.05 (br s, 2H), 7.20 (br d, J=3.18 Hz, 3H), 7.25-7.36 (m, 4H), 7.66-7.77 (m, 2H), 7.94 (d, J=1.71 Hz, 1 H), 8.01 (d, J=5.62 Hz, 1H), 8.26 (dd, J=8.31, 1.71 Hz, 1H), 9.02 (dd, J=4.16, 1.71 Hz, 1 H). LC-MS: (ES) m/z 708.3 (M+H⁺).

Step b) To a solution of benzyl N-cyclopentyl-N-[4-[(2R,3S)-3-[[4-methyl-3-(trifluoromethyl) phenyl]carbamoyl]-1-(1,7-naphthyridin-8-yl)-2-piperidyl]phenyl]carbamate (30 mg, 42.39 mol) in DCM (2 mL) was added HBr (in HOAc) (89.40 mg, 364.62 mol, 60.00 uL, 33% purity) at 0° C. Then the mixture was stirred at 20° C. for 1 h. The reaction mixture was diluted with DCM (4 mL), alkalified to pH=9-10 by saturated NaHCO₃ solution and extracted with DCM (2×3 mL). The combined organic layers were dried, filtered and concentrated in vacuo to give the residue. The residue was purified by prep-TLC (Petroleum ether/EtOAc=3/2) to give (2R,3S)-2-[4-(cyclopentylamino)phenyl]-N-[4-methyl-3-(trifluoromethyl)phenyl]-1-(1,7-naphthyridin-8-yl)piperidine-3-carboxamide (8 mg, 12.83 umol, 30.27% yield, 92% purity) as off-white solid. ¹H NMR (400 MHz, CDCl₃) δ 1.35-1.47 (m, 2H), 1.53-1.64 (m, 5H), 1.90-2.02 (m, 3H), 2.34-2.50 (m, 5H), 3.34 (br s, 1 H), 3.68 (quin, J=6.15 Hz, 1H), 3.79-3.91 (m, 1H), 4.26 (br d, J=12.80 Hz, 1H), 6.45 (d, J=8.53 Hz, 2H), 6.85 (s, 1H), 7.05 (d, J=8.28 Hz, 2H), 7.14 (d, J=5.52 Hz, 1H), 7.21 (br d, J=8.28 Hz, 1H), 7.55 (dd, J=8.28, 4.27 Hz, 1H), 7.76 (s, 1H), 7.87 (br d, J=8.28 Hz, 1H), 8.10 (dd, J=8.28, 1.51 Hz, 1H), 8.24 (d, J=5.52 Hz, 1H), 8.77 (dd, J=4.02, 1.51 Hz, 1H), 11.50 (s, 1H). LC-MS: (ES) m/z 574.3 (M+H⁺).

### Example S8: (2R,3R)-2-(4-(cyclopentylamino)phenyl)-N-(4-methyl-3-(trifluoromethyl)phenyl)-1-(pyrido[3,2-d]pyrimidin-4-yl)piperidine-3-carboxamide (Compound No. 7)

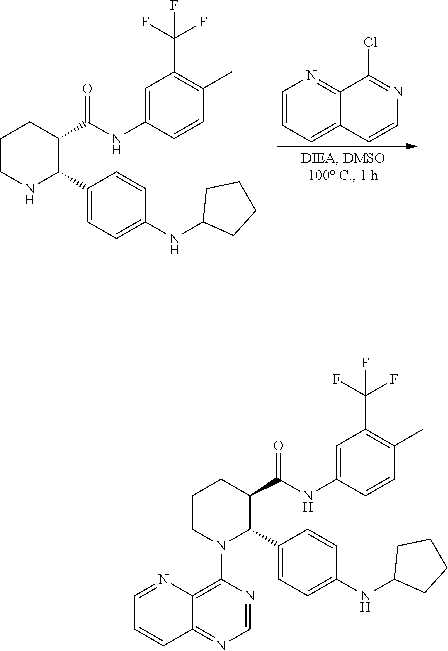 [SMILES: Cc1ccc(NC(=O)[C@H]2CCCN[C@H]2c2ccc(NC3CCCC3)cc2)cc1C(F)(F)F ; Clc1nccc2cccnc12 ; Cc1ccc(NC(=O)[C@@H]2CCCN(c3ncnc4cccnc34)[C@H]2c2ccc(NC3CCCC3)cc2)cc1C(F)(F)F]

DIEA (116.18 mg, 898.95 umol, 156.58 μL) was added to a solution of 4-chloro pyrido[3,2-d]pyrimidine (74.42 mg, 449.48 mol) and (2R,3S)-2-[4-(cyclopentylamino) phenyl]-N-[4-methyl-3-(trifluoromethyl)phenyl]piperidine-3-carboxamide (150 mg, 299.65 mol) in DMSO (1 mL). The solution was stirred at 100° C. for 1 h. The reaction mixture was added to 2 mL of H₂O, and was extracted with DCM (5 mL×2). The combined organic phase was dried with anhydrous Na₂SO₄ and filtered. The filtrate was evaporated under vacuum to give a residue. The residue was purified by column chromatography (SiO₂, Petroleum ether/Ethyl acetate=1/0 to 0:1) (plate, Petroleum ether/Ethyl acetate=1:1). The resulting product was purified again by prep-TLC (Petroleum ether/Ethyl acetate=1:1) to give compound (2R,3R)-2-[4-(cyclopentylamino)phenyl]-N-[4-methyl-3-(trifluoromethyl)phenyl]-1-pyrido[3,2-d]pyrimidin-4-yl-piperidine-3-carboxamide (18 mg, 28.82 mol, 9.62% yield, 92% purity) as a yellow solid. ¹H NMR (400 MHz, CDCl₃) δ 0.60-0.93 (2H, m), 1.06-1.43 (6H, m), 1.44-1.78 (13H, m), 1.80-2.05 (3H, m), 2.26-2.52 (5 H, m), 3.37-3.55 (2H, m), 3.58-3.71 (1H, m), 5.02 (1 H, br s), 6.44 (2H, br d, J=7.58 Hz), 6.89 (2 H, br s), 7.10 (1 H, brd, J=8.31 Hz), 7.23-7.47 (1H, m), 7.51-7.70 (3H, m), 8.15 (1H, br d, J=8.31 Hz), 8.70 (2 H, br s), 10.42 (1 H, br s). LC-MS: (ES) m/z 575.3 (M+H⁺).

### Example S9: (2R,3S)-2-(4-(cyclopentylamino)phenyl)-N-(4-methyl-3-(trifluoromethyl)phenyl)-1-(pyrido[3,4-d]pyrimidin-4-yl)piperidine-3-carboxamide (Compound No. 162)

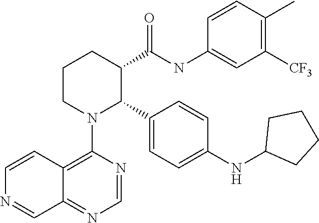 [SMILES: Cc1ccc(NC(=O)[C@H]2CCCN(c3ncnc4cnccc34)[C@H]2c2ccc(NC3CCCC3)cc2)cc1C(F)(F)F]

The title compound was synthesized in similar fashion as Example S8. ¹H NMR (400 MHz, CDCl₃) δ 1.47 (br dd, J=12.55, 6.53 Hz, 2H), 1.61-1.66 (m, 2H), 1.71-1.77 (m, 3H), 1.86 (br s, 1H), 1.95-2.06 (m, 2H), 2.07-2.15 (m, 1H), 2.31 (br s, 1H), 2.43 (d, J=1.00 Hz, 3H), 3.35 (br s, 1H), 3.64-3.83 (m, 2H), 4.06-4.30 (m, 1H), 6.34 (br s, 1H), 6.59 (br d, J=8.53 Hz, 2H), 7.12 (d, J=8.53 Hz, 2H), 7.20-7.25 (m, 1H), 7.67 (br d, J=8.53 Hz, 1H), 7.71 (s, 1H), 7.76 (br d, J=5.77 Hz, 1H), 8.54-8.60 (m, 1H), 8.82-8.87 (m, 1 H), 9.34-9.38 (m, 1H). LC-MS: (ES) m/z 575.3 (M+H⁺).

### Example S10: Synthesis of benzyl cyclopentyl(4-((2R,3S)-3-((4-methyl-3-(trifluoromethyl)-phenyl)carbamoyl)-1-(pyrido[3,4-d]pyrimidin-4-yl)piperidin-2-yl)phenyl)carbamate (Compound 167)

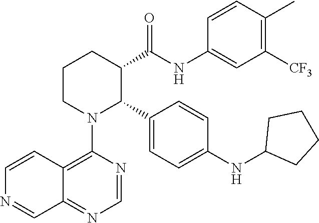 [SMILES: Cc1ccc(NC(=O)[C@H]2CCCN(c3ncnc4cnccc34)[C@H]2c2ccc(NC3CCCC3)cc2)cc1C(F)(F)F]

The title compound was synthesized in similar fashion as Example S8. ¹H NMR (400 MHz, CDCl₃) δ 1.35-1.46 (m, 2H), 1.50 (br s, 4H), 1.74-1.80 (m, 1H), 1.88 (br s, 2 H), 1.96 (br d, J=13.45 Hz, 1H), 2.22 (br d, J=13.21 Hz, 1H), 2.32-2.41 (m, 1H), 2.44 (s, 3 H), 3.35 (dt, J=12.23, 4.16 Hz, 1H), 3.43-3.53 (m, 1H), 4.24 (br d, J=13.21 Hz, 1H), 4.45-4.57 (m, 1H), 5.10 (s, 2H), 6.52 (br d, J=4.16 Hz, 1H), 7.12 (d, J=8.56 Hz, 2H), 7.17 (br s, 2H), 7.20-7.26 (m, 4H), 7.52 (d, J=8.31 Hz, 2H), 7.62 (br d, J=9.29 Hz, 1H), 7.67 (d, J=5.87 Hz, 1H), 7.75 (s, 1H), 8.06 (s, 1H), 8.59 (d, J=5.62 Hz, 1H), 8.86 (s, 1H), 9.36 (s, 1 H). LC-MS: (ES) m/z 709.3 (M+H⁺).

### Example S11: Synthesis of benzyl cyclopentyl(4-((2R,3S)-3-((4-methyl-3-(trifluoromethyl)-phenyl)carbamoyl)-1-(pyrido[3,4-b]pyrazin-5-yl)piperidin-2-yl)phenyl)carbamate (Compound No. 165)

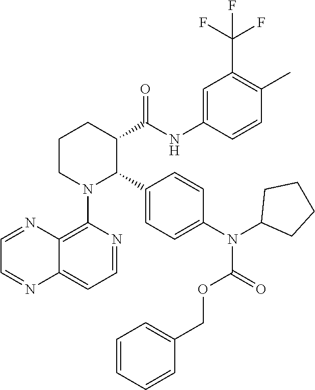 [SMILES: Cc1ccc(NC(=O)[C@H]2CCCN(c3nccc4nccnc34)[C@H]2c2ccc(N(C(=O)OCc3ccccc3)C3CCCC3)cc2)cc1C(F)(F)F]

The title compound was synthesized in similar fashion as the above examples. ¹H NMR (400 MHz, METHANOL-d₄) δ 1.30-1.36 (m, 2H), 1.39-1.43 (m, 3H), 1.79 (br s, 2 H), 1.87-2.16 (m, 4H), 2.26-2.47 (m, 5H), 3.30-3.36 (m, 1H), 3.43-3.55 (m, 1H), 4.41 (quin, J=8.38 Hz, 1H), 4.50-4.62 (m, 1H), 4.93-5.02 (m, 2H), 6.76 (br d, J=4.65 Hz, 1 H), 6.97 (d, J=8.31 Hz, 2H), 7.06 (br s, 2H), 7.17 (br d, J=2.93 Hz, 3H), 7.23 (d, J=8.56 Hz, 1H), 7.27 (d, J=5.87 Hz, 1H), 7.50 (d, J=8.31 Hz, 2H), 7.57 (br d, J=8.31 Hz, 1H), 7.83 (d, J=1.71 Hz, 1H), 8.24 (d, J=5.87 Hz, 1H), 8.82 (d, J=1.47 Hz, 1H), 8.89 (d, J=1.71 Hz, 1H). LC-MS: (ES) m/z 709.3 (M+H⁺).

### Example S12: Synthesis of (2R,3S)-2-(4-(cyclopentylamino)phenyl)-1-((2,4-dimethylphenyl)-sulfonyl)-N-(4-methyl-3-(trifluoromethyl)phenyl)piperidine-3-carboxamide (Compound No. 9)

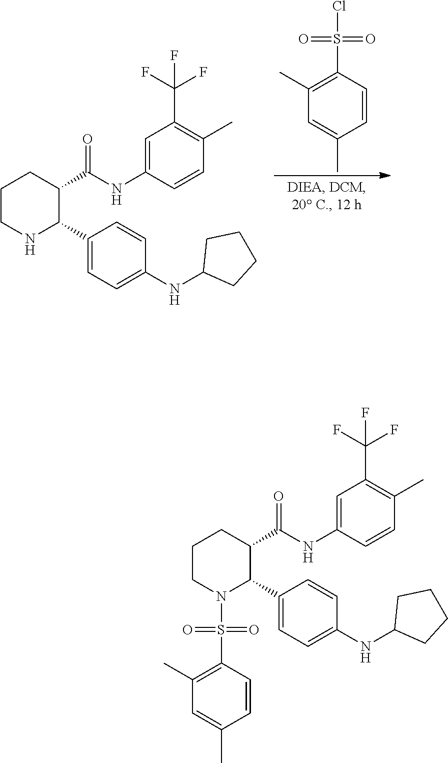 [SMILES: Cc1ccc(NC(=O)[C@H]2CCCN[C@H]2c2ccc(NC3CCCC3)cc2)cc1C(F)(F)F ; Cc1ccc(S(=O)(=O)Cl)c(C)c1 ; Cc1ccc(S(=O)(=O)N2CCC[C@H](C(=O)Nc3ccc(C)c(C(F)(F)F)c3)[C@@H]2c2ccc(NC3CCCC3)cc2)c(C)c1]

To a mixture of (2R,3S)-2-[4-(cyclopentylamino)phenyl]-N-[4-methyl-3-(trifluoromethyl) phenyl]piperidine-3-carboxamide (50 mg, 112.23 mol) and DIEA (21.76 mg, 168.35 mol, 29.32 μL) in DCM (1.5 mL) was added dropwise of a solution of 2,4-dimethylbenzenesulfonyl chloride (20.67 mg, 101.01 mol, 13.68 μL) in DCM (0.5 mL) at 20° C. Then the mixture was stirred at 20° C. for 12 h. The mixture was concentrated in vacuo to give the crude product. The crude product was purified by prep-HPLC (column: Agela ASB 150×25 mm×5 μm; mobile phase: [water (0.05% HCl)-ACN]; B %: 50%-80%, 8 min) to give (2R,3S)-2-[4-(cyclopentylamino)phenyl]-1-(2,4-dimethylphenyl)sulfonyl-N-[4-methyl-3-(trifluoromethyl)phenyl]piperidine-3-carboxamide (15 mg, 24.44 mol, 21.78% yield, 100% purity) as white solid. ¹H NMR (400 MHz, CDCl₃) δ 1.53-1.63 (m, 3H), 1.65-1.83 (m, 4H), 1.84-1.98 (m, 3H), 2.04-2.13 (m, 1H), 2.14-2.28 (m, 1H), 2.36 (s, 3H), 2.39 (s, 3H), 2.46 (s, 3H), 3.00-3.12 (m, 1H), 3.18 (dt, J=12.74, 4.67 Hz, 1H), 3.67 (quin, J=6.15 Hz, 1H), 3.77 (br d, J=11.29 Hz, 1H), 5.38 (br, d, J=5.27 Hz, 1H), 6.66 (br d, J=6.27 Hz, 2H), 6.99-7.10 (m, 4H), 7.14 (d, J=8.28 Hz, 1H), 7.42 (br d, J=8.28 Hz, 1H), 7.53 (s, 1H), 7.67 (br s, 1H), 7.79-7.88 (m, 1H). LC-MS: (ES) m/z 614.3 (M+H⁺).

### Example S13: Synthesis of (2R,3S)-2-(4-(cyclopentylamino)phenyl)-1-((2,5-dimethylphenyl)-sulfonyl)-N-(4-methyl-3-(trifluoromethyl)phenyl)piperidine-3-carboxamide (Compound No. 10)

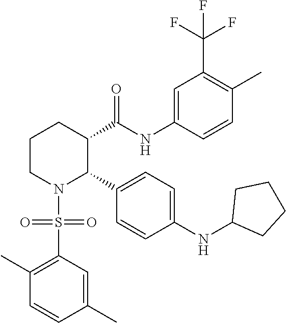 [SMILES: Cc1ccc(C)c(S(=O)(=O)N2CCC[C@H](C(=O)Nc3ccc(C)c(C(F)(F)F)c3)[C@@H]2c2ccc(NC3CCCC3)cc2)c1]

The title compound was synthesized in similar fashion as Example S12. ¹H NMR (400 MHz, CDCl₃) δ 1.46 (2 H, br s), 1.79 (11 H, br s), 1.95-2.12 (2H, m), 2.27 (3 H, s), 2.39 (6H, br d, J=19.32 Hz), 2.89 (1 H, br t, J=13.45 Hz), 3.13 (1 H, br s), 3.58 (1 H, br s), 3.76 (1H, br d, J=14.18 Hz), 5.56 (1 H, br s), 7.11 (2H, br d, J=7.83 Hz), 7.19-7.25 (2H, m), 7.28 (2 H, br s), 7.43 (1H, br d, J=8.07 Hz), 7.63 (1 H, br s), 7.79 (1 H, br s), 8.00 (1 H, br s), 10.99 (1 H, br s). LC-MS: (ES) m/z 614.3 (M+H⁺).

### Example S14: Synthesis of (2R,3S)-2-(4-(cyclopentylamino)phenyl)-1-((2,6-dimethylphenyl)-sulfonyl)-N-(4-methyl-3-(trifluoromethyl)phenyl)piperidine-3-carboxamide (Compound No. 11)

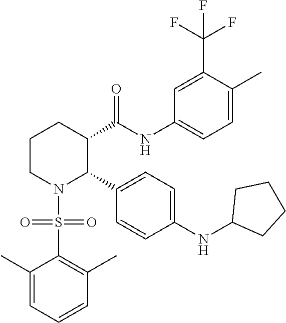 [SMILES: Cc1ccc(NC(=O)[C@H]2CCCN(S(=O)(=O)c3c(C)cccc3C)[C@H]2c2ccc(NC3CCCC3)cc2)cc1C(F)(F)F]

The title compound was synthesized in similar fashion as Example S12. ¹H NMR (400 MHz, CDCl₃) δ 1.35-1.57 (m, 5H), 1.66 (br s, 2H), 1.74-1.91 (m, 4H), 2.16 (br d, J=10.3 Hz, 1H), 2.35 (s, 3H), 2.56 (s, 6H), 3.05-3.15 (m, 1H), 3.18-3.32 (m, 2H), 3.65 (br s, 1H), 5.36 (br s, 1H), 6.20-6.74 (m, 1H), 6.77-7.20 (m, 2H), 7.23 (d, J=7.8 Hz, 2 H), 7.30 (d, J=8.3 Hz, 1H), 7.36-7.43 (m, 1H), 7.54 (br d, J=8.3 Hz, 1H), 7.74 (d, J=1.8 Hz, 1H), 10.10 (br s, 1H). LC-MS: (ES) m/z 614.4 (M+H⁺).

### Example S15: Synthesis of (2R,3S)-2-(4-(cyclopentylamino)phenyl)-1-((3,5-dimethylphenyl)-sulfonyl)-N-(4-methyl-3-(trifluoromethyl)phenyl)piperidine-3-carboxamide (Compound No. 12)

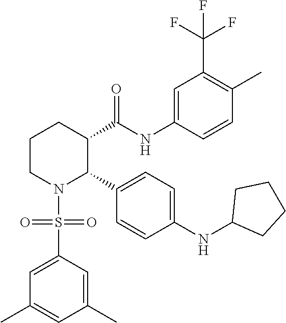 [SMILES: Cc1cc(C)cc(S(=O)(=O)N2CCC[C@H](C(=O)Nc3ccc(C)c(C(F)(F)F)c3)[C@@H]2c2ccc(NC3CCCC3)cc2)c1]

The title compound was synthesized in similar fashion as Example S12. ¹H NMR (400 MHz, CDCl₃) δ 1.59 (br d, J=6.39 Hz, 5H), 1.73-1.84 (m, 3H), 1.86-2.04 (m, 4H), 2.22 (s, 6H), 2.39 (s, 3H), 2.87-3.02 (m, 2H), 3.65 (br t, J=5.73 Hz, 1H), 3.82 (br d, J=10.14 Hz, 1H), 5.73 (br s, 1H), 6.61 (br s, 2H), 7.00-7.18 (m, 4H), 7.27-7.34 (m, 1H), 7.43 (br d, J=7.72 Hz, 1H), 7.53 (br s, 1H), 7.96 (br s, 1H). LC-MS: (ES) m/z 614.3 (M+H⁺).

### Example S16: Synthesis of (2R,3S)-2-(4-(cyclopentylamino)phenyl)-1-(mesitylsulfonyl)-N-(4-methyl-3-(trifluoromethyl)phenyl)piperidine-3-carboxamide (Compound No. 13)

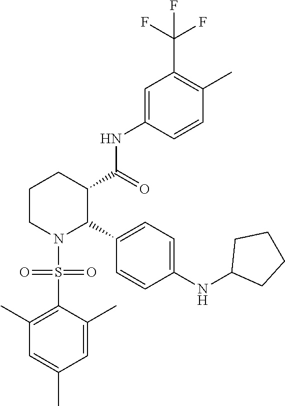

The title compound was synthesized in similar fashion as Example S12. ¹H NMR (400 MHz, METHANOL-d₄) δ 1.62-1.74 (m, 4H), 1.82 (br s, 2H), 1.95 (br d, J=6.75 Hz, 4 H), 2.24 (br d, J=12.51 Hz, 1H), 2.31 (s, 3H), 2.39 (s, 3H), 2.59 (s, 6H), 3.20-3.28 (m, 1 H), 3.42 (br d, J=8.38 Hz, 2H), 3.89 (brt, J=6.63 Hz, 1H), 4.82 (br s, 1H), 5.48 (br d, J=6.25 Hz, 1H), 7.03 (s, 2H), 7.21-7.31 (m, 3H), 7.41 (br d, J=8.25 Hz, 1H), 7.53 (br d, J=7.88 Hz, 2H), 7.69 (s, 1H), 10.06 (s, 1H). LC-MS: (ES) m/z 628.3 (M+H⁺).

### Example S17: Synthesis of (2R,3S)-2-(4-(cyclopentylamino)phenyl)-N-(4-methyl-3-(trifluoromethyl)phenyl)-1-((2-nitrophenyl)sulfonyl)piperidine-3-carboxamide (Compound No. 14)

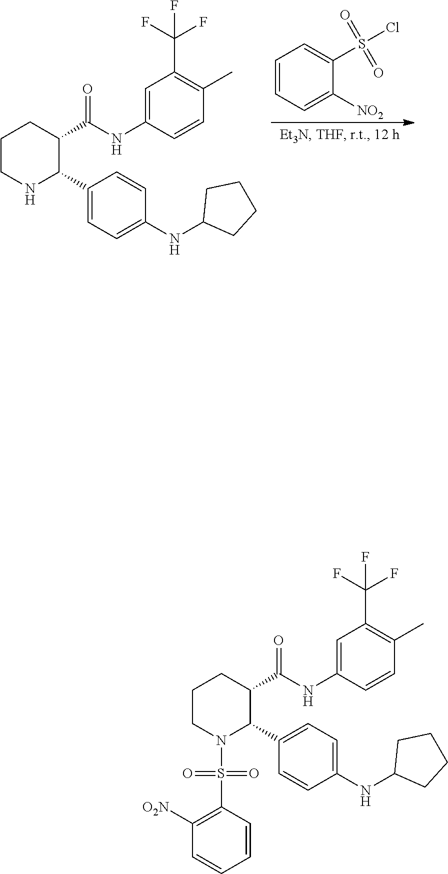

The title compound was synthesized in similar fashion as Example S12. ¹H NMR (400 MHz, CDCl₃) δ 1.49 (br s, 2H), 1.71 (br s, 2H), 1.77-1.87 (m, 6H), 1.94-2.09 (m, 2 H), 2.41 (s, 3H), 3.00-3.15 (m, 2H), 3.59-3.70 (m, 1H), 3.88 (br d, J=11.74 Hz, 1H), 5.71 (br d, J=4.65 Hz, 1H), 7.19 (d, J=8.07 Hz, 1H), 7.45 (br s, 3H), 7.50 (br d, J=7.83 Hz, 1H), 7.60-7.75 (m, 4H), 7.98 (br d, J=7.34 Hz, 1H), 8.15 (br s, 1H), 11.06 (br s, 1H). LC-MS: (ES) m/z 631.3 (M+H⁺).

### Example S18: Synthesis of (2R,3S)-2-(4-(cyclopentylamino)phenyl)-1-((4-fluoro-2-methylphenyl)-sulfonyl)-N-(4-methyl-3-(trifluoromethyl)phenyl)piperidine-3-carboxamide (Compound No. 15)₁

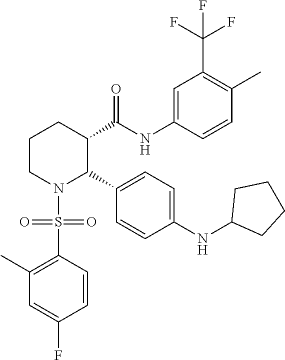

The title compound was synthesized in similar fashion as Example S12. ¹H NMR (400 MHz, CDCl₃) δ 1.49 (br s, 2H), 1.82 (br s, 8H), 2.01-2.14 (m, 2H), 2.41 (s, 3H), 2.50 (s, 3H), 2.91-3.05 (m, 1H), 3.18 (br d, J=4.52 Hz, 1H), 3.61 (br s, 1H), 3.78 (br d, J=11.29 Hz, 1H), 5.54 (br d, J=4.52 Hz, 1H), 6.94-7.02 (m, 2H), 7.18 (br d, J=8.03 Hz, 3 H), 7.31 (br s, 1H), 7.42 (br d, J=7.78 Hz, 1H), 7.62 (s, 1H), 7.81 (br s, 1H), 7.91-8.04 (m, 1H), 10.85 (br s, 1H). LC-MS: (ES) m/z 618.3 (M+H⁺).

### Example S19: Synthesis of (2R,3S)-1-((3-chloro-2-methylphenyl)sulfonyl)-2-(4-(cyclopentyl-amino)phenyl)-N-(4-methyl-3-(trifluoromethyl)phenyl)piperidine-3-carboxamide (Compound No. 16)

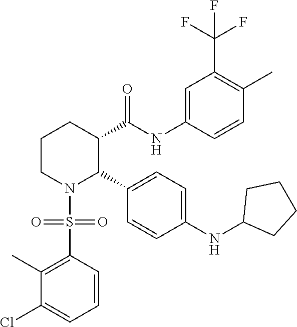

The title compound was synthesized in similar fashion to Example S12. ¹H NMR (400 MHz, CDCl₃) δ 1.51 (br s, 2H), 1.74 (br d, J=10.80 Hz, 5H), 1.83-1.95 (m, 3H), 2.01-2.18 (m, 2H), 2.40 (br s, 3H), 2.51 (s, 3H), 3.02-3.22 (m, 2H), 3.64 (br t, J=6.50 Hz, 1 H), 3.80 (br d, J=12.35 Hz, 1H), 5.45 (br d, J=3.97 Hz, 1H), 7.09 (br s, 4H), 7.14-7.24 (m, 2H), 7.38 (br d, J=8.38 Hz, 1H), 7.48-7.59 (m, 2H), 7.70 (br s, 1H), 7.87 (br d, J=7.72 Hz, 1H). LC-MS: (ES) m/z 634.2 (M+H⁺).

### Example S20: Synthesis of (2R,3S)-2-(4-(cyclopentylamino)phenyl)-1-((5-fluoro-2-methylphenyl)-sulfonyl)-N-(4-methyl-3-(trifluoromethyl)phenyl)piperidine-3-carboxamide (Compound No. 17)

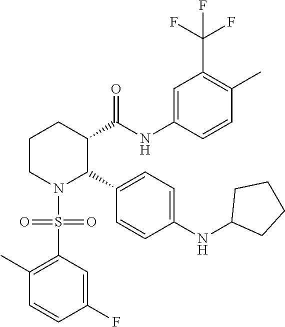

The title compound was synthesized in similar fashion as Example S12. ¹H NMR (400 MHz, METHANOL-d₄) δ 1.59-1.75 (m, 5H), 1.82 (br s, 2H), 1.89-2.08 (m, 4H), 2.23 (qd, J=13.53, 3.91 Hz, 1H), 2.39 (s, 3H), 2.51 (s, 3H), 3.22 (ddd, J=12.90, 6.30, 4.03 Hz, 1H), 3.45 (td, J=13.14, 2.57 Hz, 1H), 3.76 (br d, J=11.98 Hz, 1H), 3.83-3.93 (m, 1H), 5.58 (d, J=6.36 Hz, 1H), 7.19-7.29 (m, 4H), 7.29-7.35 (m, 1H), 7.42 (dd, J=8.19, 1.83 Hz, 1H), 7.46-7.56 (m, 3H), 7.71 (d, J=1.96 Hz, 1H). LC-MS: (ES) m/z 618.1 (M+H⁺).

### Example S21: Synthesis of (2R,3S)-1-((3-fluoro-2-methylphenyl)sulfonyl)-2-(4-(cyclopentyl-amino)phenyl)-N-(4-methyl-3-(trifluoromethyl)phenyl)piperidine-3-carboxamide (Compound No. 18)

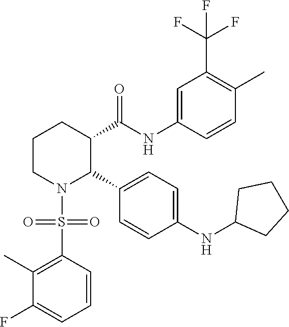

The title compound was synthesized in similar fashion as Example S12. ¹H NMR (400 MHz, CDCl₃) δ 1.60-2.02 (m, 10H), 2.12 (br s, 2H), 2.44 (br s, 6H), 3.18 (br s, 2H), 3.57-4.02 (m, 2H), 5.54 (br s, 1H), 7.27 (br s, 7H), 7.45 (br s, 1H), 7.62 (br s, 1H), 7.70-8.13 (m, 1H). LC-MS: (ES) m/z 618.2 (M+H⁺).

### Example S22: Synthesis of (2R,3S)-2-(4-(cyclopentylamino)phenyl)-1-((2,6-difluorophenyl)-sulfonyl)-N-(4-methyl-3-(trifluoromethyl)phenyl)piperidine-3-carboxamide (Compound No. 19)

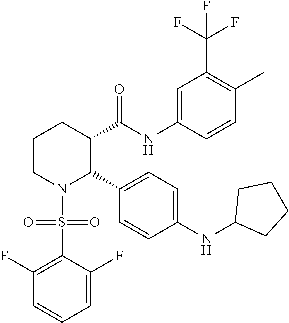

The title compound was synthesized in similar fashion as Example S12. ¹H NMR (400 MHz, CDCl₃) δ 1.51-1.67 (m, 2H), 1.73-1.94 (m, 8H), 1.96-2.11 (m, 2H), 2.38 (s, 3 H), 3.03 (br s, 1H), 3.16 (br t, J=12.96 Hz, 1H), 3.63 (br s, 1H), 4.04 (br d, J=11.49 Hz, 1 H), 5.71 (br d, J=4.40 Hz, 1H), 6.84 (brt, J=8.80 Hz, 2H), 7.15 (br d, J=7.58 Hz, 1H), 7.18-7.26 (m, 2H), 7.28-7.31 (m, 1H), 7.38 (br t, J=7.58 Hz, 2H), 7.59 (s, 1H), 7.98 (br s, 1 H), 10.12-11.64 (m, 1H). LC-MS: (ES) m/z 622.3 (M+H⁺).

### Example S23: Synthesis of (2R,3S)-2-(4-(cyclopentylamino)phenyl)-1-((2,6-dichlorophenyl)-sulfonyl)-N-(4-methyl-3-(trifluoromethyl)phenyl)piperidine-3-carboxamide (Compound No. 20)

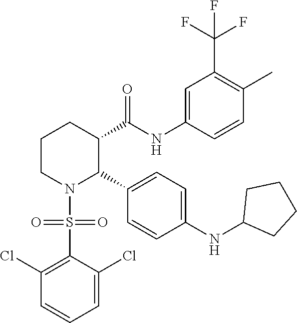

The title compound was synthesized in similar fashion as Example S12. ¹H NMR (400 MHz, CDCl₃) δ 1.50 (br s, 2H), 1.65 (br s, 2H), 1.77-1.94 (m, 6H), 2.04 (br d, J=16.54 Hz, 2H), 2.40 (br s, 3H), 3.09-3.27 (m, 2H), 3.62 (br s, 1H), 4.03 (br d, J=13.67 Hz, 1H), 5.57 (br d, J=4.85 Hz, 1H), 7.18 (br d, J=7.94 Hz, 3H), 7.25 (br s, 1H), 7.30 (s, 1 H), 7.35-7.46 (m, 3H), 7.60 (s, 1H), 7.72 (br s, 1H), 9.53-11.11 (m, 1H). LC-MS: (ES) m/z 654.1 (M+H⁺).

### Example S24: Synthesis of methyl 2-(((2R,3S)-2-(4-(cyclopentylamino)phenyl)-3-((4-methyl-3-(trifluoromethyl)phenyl)carbamoyl)piperidin-1-yl)sulfonyl)-3-methylbenzoate (Compound No. 21)

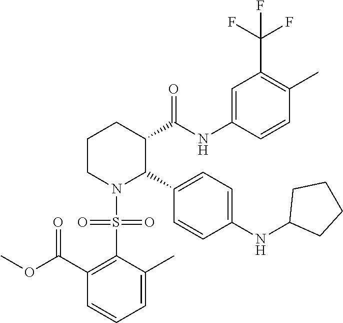

The title compound was synthesized in similar fashion as Example S12. ¹H NMR (400 MHz, METHANOL-d₄) δ 1.59-1.73 (m, 5H), 1.76-1.88 (m, 2H), 1.89-2.03 (m, 4 H), 2.26 (qd, J=13.55, 4.02 Hz, 1H), 2.39 (d, J=1.51 Hz, 3H), 2.56 (s, 3H), 3.24 (ddd, J=13.05, 6.02, 3.76 Hz, 1H), 3.32-3.37 (m, 1H), 3.82-3.91 (m, 1H), 3.95 (s, 4H), 5.57 (d, J=6.27 Hz, 1H), 7.20-7.28 (m, 3H), 7.34 (d, J=7.03 Hz, 1H), 7.41-7.50 (m, 4H), 7.54-7.60 (m, 1H), 7.75-7.79 (m, 1H), 9.82 (s, 1H). LC-MS: (ES) m/z 680.1 (M+Na).

### Example S25: Synthesis of (2R,3S)-2-(4-(cyclopentylamino)phenyl)-N-(4-methyl-3-(trifluoro-methyl)phenyl)-1-(o-tolylsulfonyl)piperidine-3-carboxamide (Compound No. 22)

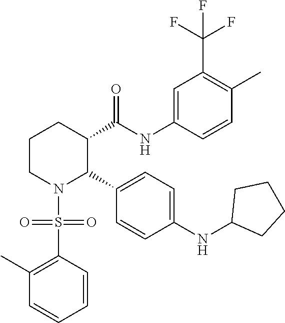

The title compound was synthesized in similar fashion as Example S12. ¹H NMR (400 MHz, CDCl₃) δ 1.56-1.89 (11H, m) 2.03 (2 H, br s) 2.37 (3 H, s) 2.46 (3 H, s) 2.91 (1 H, brt, J=12.96 Hz) 3.15 (1 H, br s) 3.57 (1 H, br s) 3.79 (1H, br d, J=12.23 Hz) 5.53 (1H, br d, J=4.16 Hz) 7.15 (3H, br d, J=6.36 Hz) 7.22 (1 H, s) 7.34 (2H, br d, J=7.34 Hz) 7.41 (2 H, brt, J=7.09 Hz) 7.63 (1 H, s) 7.93 (1H, br d, J=7.83 Hz) 8.06 (1 H, br s) 11.12 (1 H, br s). LC-MS: (ES) m/z 622.3 (M+Na).

### Example S26: Synthesis of (2R,3S)-2-(4-(cyclopentylamino)phenyl)-1-((2-methoxyphenyl)-sulfonyl)-N-(4-methyl-3-(trifluoromethyl)phenyl)piperidine-3-carboxamide (Compound No. 23)

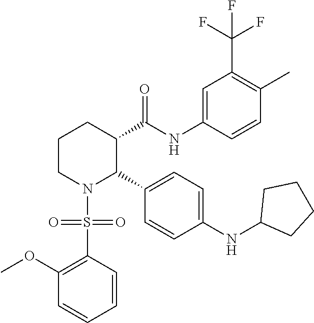

The title compound was synthesized in similar fashion as Example S12. ¹H NMR (400 MHz, METHANOL-d₄) δ 1.64 (td, J=12.84, 5.14 Hz, 5H), 1.81 (br d, J=5.62 Hz, 2H), 1.89-2.06 (m, 4H), 2.11-2.25 (m, 1H), 2.38 (s, 3H), 3.14 (ddd, J=12.96, 6.24, 3.79 Hz, 1 H), 3.44-3.55 (m, 1H), 3.78-3.87 (m, 4H), 3.99-4.09 (m, 1H), 5.57 (d, J=6.36 Hz, 1H), 6.89-6.99 (m, 2H), 7.15 (br d, J=8.07 Hz, 2H), 7.23 (d, J=8.31 Hz, 1H), 7.38-7.49 (m, 4 H), 7.69 (d, J=1.96 Hz, 1H), 7.78 (d, J=8.07 Hz, 1H). LC-MS: (ES) m/z 616.2 (M+H⁺).

### Example S27: Synthesis of (2R,3S)-2-(4-(cyclopentylamino)phenyl)-N-(4-methyl-3-(trifluoro-methyl)phenyl)-1-((2-(trifluoromethoxy)phenyl)sulfonyl)piperidine-3-carboxamide (Compound No. 24)

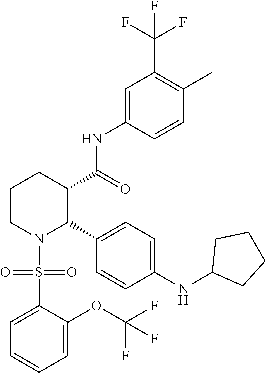

The title compound was synthesized in similar fashion as Example S12. ¹H NMR (400 MHz, CDCl₃) δ 1.48 (2 H, br s) 1.72-2.07 (11H, m) 2.37 (3 H, br s) 2.91-3.09 (2H, m) 3.58 (1 H, br s) 3.88 (1H, br d, J=12.47 Hz) 5.61 (1H, br d, J=5.14 Hz) 7.14 (1H, br d, J=8.07 Hz) 7.22 (2 H, br s) 7.25-7.41 (4H, m) 7.45-7.53 (1H, m) 7.58 (1 H, s) 7.73-7.87 (2H, m) 11.07 (1 H, br s). LC-MS: (ES) m/z 670.2 (M+H⁺).

### Example S28: Synthesis of (2R,3S)-2-(4-(cyclopentylamino)phenyl)-1-((2-fluorophenyl)sulfonyl)-N-(4-methyl-3-(trifluoromethyl)phenyl)piperidine-3-carboxamide (Compound No. 25)

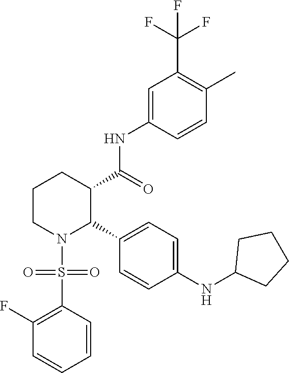

The title compound was synthesized in similar fashion as Example S12. ¹H NMR (400 MHz, CDCl₃) δ 1.60-2.07 (12H, m) 2.36 (3 H, s) 2.91-3.02 (1H, m) 3.09 (1 H, brt, J=12.96 Hz) 3.61 (1 H, quin, J=6.48 Hz) 3.89 (1H, br d, J=11.00 Hz) 5.64 (1H, br d, J=5.14 Hz) 6.90 (2 H, br s) 7.01-7.19 (6H, m) 7.34-7.46 (2H, m) 7.54 (1 H, s) 7.68 (1 H, brt, J=7.09 Hz) 7.87 (1 H, br s). LC-MS: (ES) m/z 604.3 (M+H⁺).

### Example S29: Synthesis of (2R,3S)-1-((2-chlorophenyl)sulfonyl)-2-(4-(cyclopentylamino)phenyl)-N-(4-methyl-3-(trifluoromethyl)phenyl)piperidine-3-carboxamide (Compound No. 26)

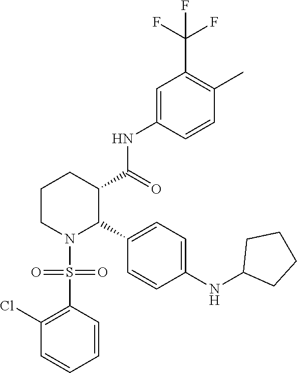

The title compound was synthesized in similar fashion as Example S12. ¹H NMR (400 MHz, CDCl₃) δ 1.38-1.61 (2H, m) 1.79 (9 H, br s) 1.98 (2 H, br s) 2.37 (3 H, s) 3.01-3.19 (2H, m) 3.59 (1H, br s) 3.85 (1H, brd, J=11.25 Hz) 5.59 (1H, brd, J=5.07 Hz) 7.15 (1H, br d, J=8.16 Hz) 7.21 (1 H, br s) 7.26-7.34 (3H, m) 7.36-7.46 (3H, m) 7.59 (1 H, s) 7.86 (1 H, br s) 8.01 (1H, br d, J=7.94 Hz) 10.46-11.32 (1H, m). LC-MS: (ES) m/z 620.2 (M+H⁺).

### Example S30: Synthesis of (2R,3S)-1-((2-bromophenyl)sulfonyl)-2-(4-(cyclopentyl-amino)phenyl)-N-(4-methyl-3-(trifluoromethyl)phenyl)piperidine-3-carboxamide (Compound No. 27)

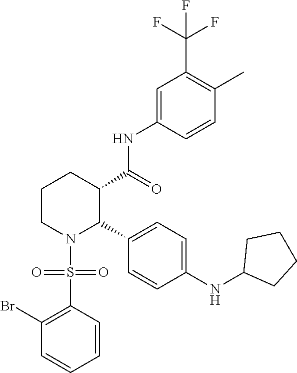

The title compound was synthesized in similar fashion as Example S12. ¹H NMR (400 MHz, METHANOL-d₄) δ 1.60-1.73 (m, 5H), 1.78-1.89 (m, 2H), 1.90-2.03 (m, 4 H), 2.13-2.30 (m, 1H), 2.38 (d, J=1.25 Hz, 3H), 3.26-3.31 (m, 1H), 3.54 (td, J=13.30, 2.51 Hz, 1H), 3.78 (br dd, J=13.55, 3.26 Hz, 1H), 3.83-3.93 (m, 1H), 5.59 (d, J=6.27 Hz, 1H), 7.22-7.29 (m, 3H), 7.40-7.44 (m, 1H), 7.44-7.53 (m, 4H), 7.68-7.75 (m, 2H), 8.11 (dd, J=7.53, 2.01 Hz, 1H), 9.98 (s, 1H). LC-MS: (ES) m/z 664.3 (M+H⁺).

### Example S31: Synthesis of (2R,3S)-2-(4-(cyclopentylamino)phenyl)-N-(4-methyl-3-(trifluoro-methyl)phenyl)-1-((2-(trifluoromethyl)phenyl)sulfonyl)piperidine-3-carboxamide (Compound No. 28)

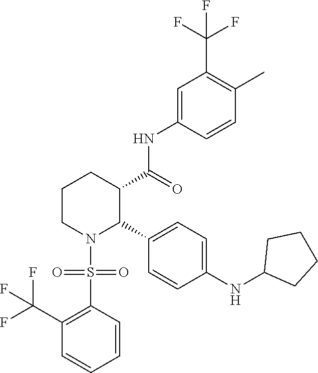

The title compound was synthesized in similar fashion as Example S12. ¹H NMR (400 MHz, METHANOL-d₄) δ 1.59-1.75 (m, 5H), 1.82 (br s, 2H), 1.88-2.07 (m, 4H), 2.12-2.27 (m, 1H), 2.39 (s, 3H), 3.23 (ddd, J=12.90, 6.17, 3.91 Hz, 1H), 3.41-3.56 (m, 1 H), 3.81-3.93 (m, 2H), 5.64 (d, J=6.11 Hz, 1H), 7.21-7.31 (m, 3H), 7.44 (dd, J=8.19, 1.83 Hz, 1H), 7.50 (br d, J=7.34 Hz, 2H), 7.67-7.78 (m, 3H), 7.89 (br d, J=7.34 Hz, 1H), 8.10 (br d, J=7.09 Hz, 1H). LC-MS: (ES) m/z 654.1 (M+H⁺).

### Example S32: Synthesis of (2R,3S)-2-(4-(cyclopentylamino)phenyl)-N-(4-methyl-3-(trifluoro-methyl)phenyl)-1-((2-(methylsulfonyl)phenyl)sulfonyl)piperidine-3-carboxamide (Compound No. 29)

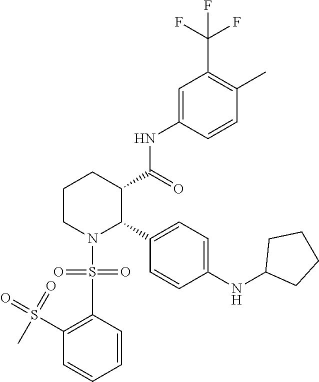 [SMILES: Cc1ccc(NC(=O)[C@H]2CCCN(S(=O)(=O)c3ccccc3S(C)(=O)=O)[C@H]2c2ccc(NC3CCCC3)cc2)cc1C(F)(F)F]

The title compound was synthesized in similar fashion as Example S12. ¹H NMR (400 MHz, CDCl₃) δ 1.63 (br d, J=11.80 Hz, 4H), 1.72-1.92 (m, 7H), 1.95-2.11 (m, 1H), 2.41 (s, 3H), 2.70-2.81 (m, 1H), 2.98 (br t, J=12.80 Hz, 1H), 3.62 (s, 3H), 3.65-3.71 (m, 1H), 3.86 (br d, J=11.54 Hz, 1H), 6.15 (d, J=4.52 Hz, 1H), 7.19 (d, J=8.28 Hz, 1H), 7.49 (br s, 1H), 7.53-7.58 (m, 1H), 7.65 (br d, J=8.28 Hz, 2H), 7.83 (d, J=2.01 Hz, 1H), 7.88 (quind, J=7.56, 7.56, 7.56, 7.56, 1.63 Hz, 2H), 8.34 (dd, J=7.53, 1.51 Hz, 1H), 8.50 (dd, J=7.53, 1.76 Hz, 1H), 8.86 (s, 1H), 11.10 (s, 1H). LC-MS: (ES) m/z 664.4 (M+H⁺).

### Example S33: Synthesis of (2R,3S)-1-((2-cyanophenyl)sulfonyl)-2-(4-(cyclopentylamino)phenyl)-N-(4-methyl-3-(trifluoromethyl)phenyl)piperidine-3-carboxamide (Compound No. 30)

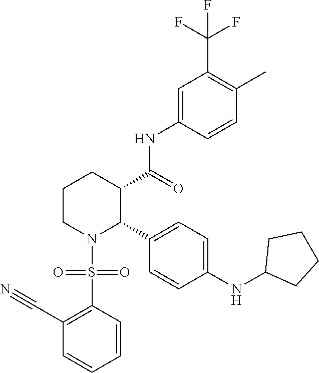 [SMILES: Cc1ccc(NC(=O)[C@H]2CCCN(S(=O)(=O)c3ccccc3C#N)[C@H]2c2ccc(NC3CCCC3)cc2)cc1C(F)(F)F]

The title compound was synthesized in similar fashion as Example S12. ¹H NMR (400 MHz, CDCl₃) δ 1.07-1.26 (1H, m) 1.48 (2 H, br s) 1.69-2.05 (10H, m) 2.40 (3 H, s) 3.14 (2 H, brt, J=11.03 Hz) 3.50-3.70 (2H, m) 5.98 (1H, br d, J=4.41 Hz) 7.18 (1H, br d, J=8.16 Hz) 7.25 (1 H, s) 7.52 (3H, br d, J=8.16 Hz) 7.63-7.79 (3H, m) 7.89 (1H, d, J=7.50 Hz) 8.09 (1H, br d, J=7.50 Hz) 8.40 (1 H, s) 10.22-11.57 (1H, m). LC-MS: (ES) m/z 611.3 (M+H⁺).

### Example S34: Synthesis of methyl2-(((2R,3S)-2-(4-(cyclopentylamino)phenyl)-3-((4-methyl-3-(trifluoromethyl)phenyl)carbamoyl)piperidin-1-yl)sulfonyl)benzoate (Compound No. 31)

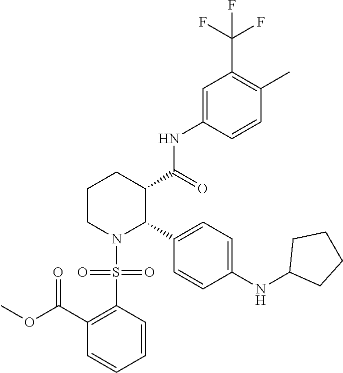 [SMILES: COC(=O)c1ccccc1S(=O)(=O)N1CCC[C@H](C(=O)Nc2ccc(C)c(C(F)(F)F)c2)[C@@H]1c1ccc(NC2CCCC2)cc1]

The title compound was synthesized in similar fashion as Example S12. ¹H NMR (400 MHz, METHANOL-d₄) δ 1.39-1.54 (m, 1H), 1.61-1.74 (m, 4H), 1.77-2.02 (m, 6 H), 2.02-2.18 (m, 1H), 2.40 (d, J=1.22 Hz, 3H), 2.99 (ddd, J=12.84, 5.62, 3.79 Hz, 1H), 3.20-3.29 (m, 1H), 3.84-3.94 (m, 1H), 3.97-4.05 (m, 4H), 5.76 (d, J=5.62 Hz, 1H), 7.25-7.31 (m, 3H), 7.50-7.64 (m, 5H), 7.65-7.71 (m, 1H), 7.82 (d, J=2.20 Hz, 1H), 7.93 (d, J=7.83 Hz, 1H), 9.86 (s, 1H). LC-MS: (ES) m/z 666.2 (M+Na).

### Example S35: Synthesis of (2R,3S)-2-(4-(cyclopentylamino)phenyl)-N-(4-methyl-3-(trifluoro-methyl)phenyl)-1-(naphthalen-2-ylsulfonyl)piperidine-3-carboxamide (Compound No. 32)

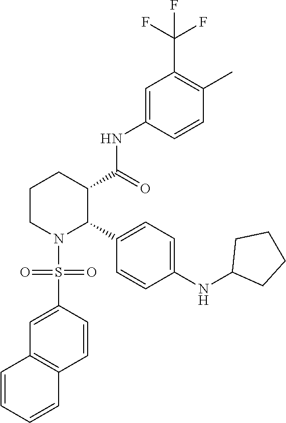 [SMILES: Cc1ccc(NC(=O)[C@H]2CCCN(S(=O)(=O)c3ccc4ccccc4c3)[C@H]2c2ccc(NC3CCCC3)cc2)cc1C(F)(F)F]

The title compound was synthesized in similar fashion as Example S12. ¹H NMR (400 MHz, CDCl₃) δ 1.46 (1 H, br s) 1.61-1.96 (13H, m) 2.25 (3 H, s) 2.71 (1H, br d, J=5.07 Hz) 2.95 (1 H, br t, J=12.68 Hz) 3.54-3.68 (1H, m) 3.96 (1H, br d, J=11.47 Hz) 6.06 (1 H, br s) 6.99 (1H, br d, J=7.94 Hz) 7.16 (2H, br d, J=8.16 Hz) 7.25 (1 H, s) 7.39 (1 H, br d, J=7.94 Hz) 7.47-7.64 (4H, m) 7.81-7.92 (3H, m) 8.27 (1 H, br s) 8.45 (1 H, br s). LC-MS: (ES) m/z 636.3 (M+H⁺).

### Example S36: Synthesis of (2R,3S)-2-(4-(cyclopentylamino)phenyl)-N-(4-methyl-3-(trifluoromethyl)phenyl)-1-(naphthalen-1-ylsulfonyl)piperidine-3-carboxamide (Compound No. 33)

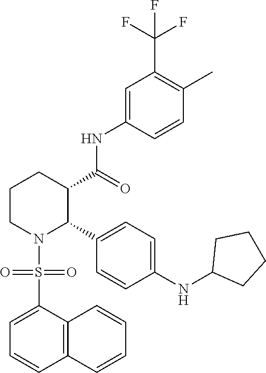 [SMILES: Cc1ccc(NC(=O)[C@H]2CCCN(S(=O)(=O)c3cccc4ccccc34)[C@H]2c2ccc(NC3CCCC3)cc2)cc1C(F)(F)F]

The title compound was synthesized in similar fashion as Example S12. ¹H NMR (400 MHz, METHANOL-d₄) δ 1.46-1.73 (m, 5H), 1.77-1.99 (m, 6H), 2.14 (qd, J=13.37, 3.42 Hz, 1H), 2.39 (s, 3H), 3.17 (ddd, J=12.90, 6.17, 3.91 Hz, 1H), 3.40 (td, J=13.14, 2.32 Hz, 1H), 3.79 (quin, J=6.91 Hz, 1H), 3.91 (br dd, J=13.57, 3.06 Hz, 1H), 5.72 (d, J=6.36 Hz, 1H), 7.10 (d, J=8.56 Hz, 2H), 7.25 (d, J=8.31 Hz, 1H), 7.37 (d, J=8.56 Hz, 2H), 7.43 (dd, J=8.31, 1.71 Hz, 1H), 7.51 (t, J=7.83 Hz, 1H), 7.56-7.68 (m, 2H), 7.71 (d, J=1.71 Hz, 1H), 7.95 (d, J=7.83 Hz, 1H), 8.10 (d, J=8.07 Hz, 1H), 8.21 (d, J=7.34 Hz, 1H), 8.58 (d, J=8.31 Hz, 1H), 10.11 (s, 1H). LC-MS: (ES) m/z 658.4 (M+Na).

### Example S37: Synthesis of (2R,3S)-2-(4-(cyclopentylamino)phenyl)-N-(4-methyl-3-(trifluoro-methyl)phenyl)-1-(phenylsulfonyl)piperidine-3-carboxamide (Compound No. 34)

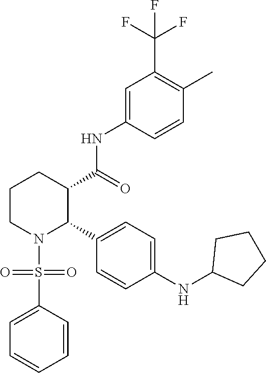 [SMILES: Cc1ccc(NC(=O)[C@H]2CCCN(S(=O)(=O)c3ccccc3)[C@H]2c2ccc(NC3CCCC3)cc2)cc1C(F)(F)F]

The title compound was synthesized in similar fashion as Example S12. ¹H NMR (400 MHz, CDCl₃) δ 1.41-1.55 (m, 2H), 1.79 (br d, J=12.57 Hz, 6H), 1.92 (br s, 4H), 2.39 (br s, 3H), 2.78-3.03 (m, 2H), 3.67 (br s, 1H), 3.86 (br d, J=11.69 Hz, 1H), 5.85 (br s, 1 H), 6.98 (br s, 2H), 7.14 (br s, 3H), 7.35-7.52 (m, 4H), 7.60 (br s, 1H), 7.68 (br d, J=7.06 Hz, 2H), 8.09 (br s, 1H). LC-MS: (ES) m/z 608.3 (M+Na).

### Example S38: Synthesis of (2R,3S)-2-(4-(cyclopentylamino)phenyl)-N-(4-methyl-3-(trifluoro-methyl)phenyl)-1-(pyridin-3-ylsulfonyl)piperidine-3-carboxamide (Compound No. 35)

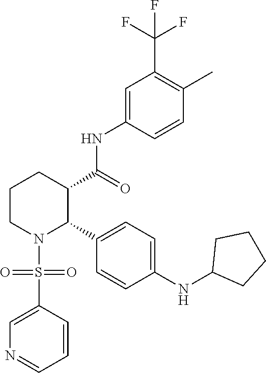 [SMILES: Cc1ccc(NC(=O)[C@H]2CCCN(S(=O)(=O)c3cccnc3)[C@H]2c2ccc(NC3CCCC3)cc2)cc1C(F)(F)F]

The title compound was synthesized in similar fashion as Example S12. ¹H NMR (400 MHz, METHANOL-d₄) δ 1.55-1.75 (4H, m) 1.76-1.88 (3H, m) 1.88-2.15 (5H, m) 2.37 (3 H, s) 3.14-3.22 (1H, m) 3.31-3.42 (1H, m) 3.80-3.93 (1H, m) 4.01 (1H, br d, J=8.80 Hz) 5.70 (1H, d, J=6.60 Hz) 7.19-7.28 (3H, m) 7.40 (1H, br d, J=8.56 Hz) 7.46 (2 H, d, J=8.31 Hz) 7.66 (1H, dd, J=8.07, 5.14 Hz) 7.71 (1 H, s) 8.25 (1H, br d, J=8.07 Hz) 8.67-8.76 (2H, m). LC-MS: (ES) m/z 587.1 (M+H⁺).

### Example S39: Synthesis of (2R,3S)-1-((2-chloropyridin-3-yl)sulfonyl)-2-(4-(cyclopentylamino)-phenyl)-N-(4-methyl-3-(trifluoromethyl)phenyl)piperidine-3-carboxamide (Compound No. 36)

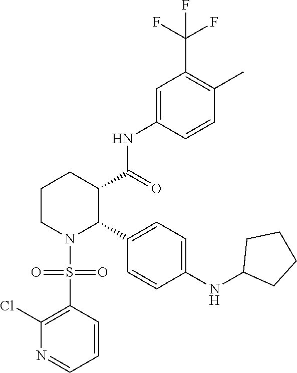 [SMILES: Cc1ccc(NC(=O)[C@H]2CCCN(S(=O)(=O)c3cccnc3Cl)[C@H]2c2ccc(NC3CCCC3)cc2)cc1C(F)(F)F]

The title compound was synthesized in similar fashion as Example S12. ¹H NMR (400 MHz, METHANOL-d₄) 61.59-1.75 (m, 5H), 1.82 (br d, J=4.89 Hz, 2H), 1.90-2.01 (m, 3H), 2.06 (br d, J=13.45 Hz, 1H), 2.16-2.30 (m, 1H), 2.38 (d, J=1.22 Hz, 3H), 3.25 (ddd, J=12.96, 6.60, 4.16 Hz, 1H), 3.65 (td, J=13.27, 2.81 Hz, 1H), 3.80-3.91 (m, 1H), 3.99 (br d, J=10.27 Hz, 1H), 5.53 (d, J=6.60 Hz, 1H), 7.23 (t, J=8.80 Hz, 3H), 7.37-7.43 (m, 1H), 7.45-7.51 (m, 3H), 7.68 (d, J=1.96 Hz, 1H), 8.37-8.50 (m, 2H), 10.08 (s, 1H). LC-MS: (ES) m/z 621.3 (M+H⁺).

### Example S40: Synthesis of (2R,3S)-2-(4-(cyclopentylamino)phenyl)-N-(4-methyl-3-(trifluoro-methyl)phenyl)-1-((perfluorophenyl)sulfonyl)piperidine-3-carboxamide (Compound No. 37)

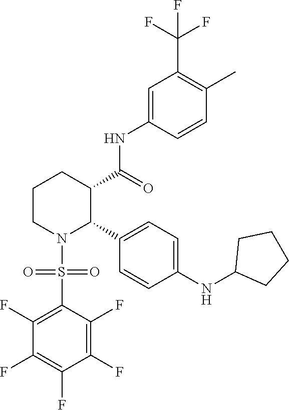 [SMILES: Cc1ccc(NC(=O)[C@H]2CCCN(S(=O)(=O)c3c(F)c(F)c(F)c(F)c3F)[C@H]2c2ccc(NC3CCCC3)cc2)cc1C(F)(F)F]

The title compound was synthesized in similar fashion as Example S12. ¹H NMR (400 MHz, METHANOL-d₄) δ 1.53-1.71 (4H, m) 1.72-1.99 (6H, m) 2.05-2.16 (2H, m) 2.37 (3 H, s) 3.17 (1H, ddd, J=12.72, 6.36, 3.67 Hz) 3.59 (1 H, br t, J=12.84 Hz) 3.77-3.87 (1H, m) 4.08-4.16 (1H, m) 5.63 (1H, d, J=6.60 Hz) 7.16 (2 H, brd, J=8.31 Hz) 7.23 (1H, d, J=8.31 Hz) 7.38 (1H, br d, J=8.31 Hz) 7.46 (2H, d, J=8.56 Hz) 7.69 (1H, d, J=1.71 Hz). LC-MS: (ES) m/z 676.2 (M+H⁺).

### Example S41: Synthesis of (2R,3S)-2-(4-(cyclopentylamino)phenyl)-N-(4-methyl-3-(trifluoro-methyl)phenyl)-1-((1,3,5-trimethyl-1H-pyrazol-4-yl)sulfonyl)piperidine-3-carboxamide (Compound No. 38)

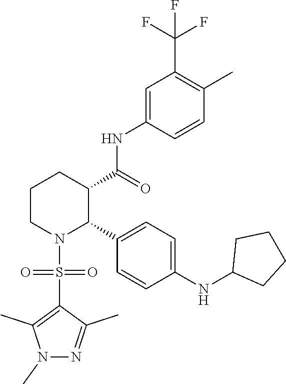 [SMILES: Cc1ccc(NC(=O)[C@H]2CCCN(S(=O)(=O)c3c(C)nn(C)c3C)[C@H]2c2ccc(NC3CCCC3)cc2)cc1C(F)(F)F]

The title compound was synthesized in similar fashion as Example S12. ¹H NMR (400 MHz, CDCl₃) δ 1.53 (br s, 1H), 1.64 (br s, 2H), 1.72-2.09 (m, 9H), 2.25 (s, 3H), 2.34 (s, 3H), 2.41 (s, 3H), 2.96 (br t, J=12.55 Hz, 1H), 3.02-3.13 (m, 1H), 3.66 (s, 4H), 3.77 (br d, J=12.30 Hz, 1H), 5.70 (br d, J=4.27 Hz, 1H), 7.17 (br d, J=8.28 Hz, 1H), 7.31 (br d, J=8.28 Hz, 2H), 7.42-7.49 (m, 2H), 7.67 (s, 1H), 8.09 (br s, 1H), 11.20 (br s, 1H). LC-MS: (ES) m/z 640.4 (M+Na).

### Example S42: Synthesis of (2R,3S)-2-(4-(cyclopentylamino)phenyl)-1-((3,5-dimethylisoxazol-4-yl)sulfonyl)-N-(4-methyl-3-(trifluoromethyl)phenyl)piperidine-3-carboxamide (Compound No. 8)

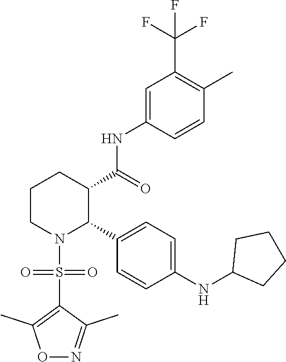 [SMILES: Cc1ccc(NC(=O)[C@H]2CCCN(S(=O)(=O)c3c(C)noc3C)[C@H]2c2ccc(NC3CCCC3)cc2)cc1C(F)(F)F]

The title compound was synthesized in similar fashion as Example S12. ¹H NMR (400 MHz, METHANOL-d₄) δ 1.58-1.74 (m, 5H), 1.76-1.92 (m, 3H), 1.92-2.04 (m, 2 H), 2.05-2.15 (m, 2H), 2.21 (s, 3H), 2.40 (s, 3H), 2.47 (s, 3H), 3.17 (ddd, J=12.65, 6.30, 3.79 Hz, 1H), 3.56 (td, J=12.90, 2.57 Hz, 1H), 3.83-3.97 (m, 2H), 5.58 (d, J=6.36 Hz, 1 H), 7.27 (br d, J=8.07 Hz, 3H), 7.42-7.47 (m, 1H), 7.52 (d, J=8.31 Hz, 2H), 7.75 (d, J=1.96 Hz, 1H). LC-MS: (ES) m/z 627.2 (M+Na).

### Example S43: Synthesis of (2R,3S)-1-(benzylsulfonyl)-2-(4-(cyclopentylamino)phenyl)-N-(4-methyl-3-(trifluoromethyl)phenyl)piperidine-3-carboxamide (Compound No. 40)

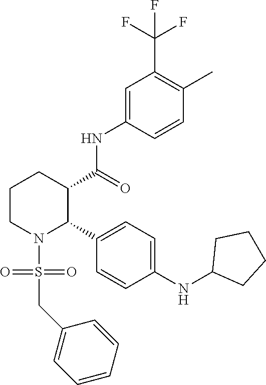 [SMILES: Cc1ccc(NC(=O)[C@H]2CCCN(S(=O)(=O)Cc3ccccc3)[C@H]2c2ccc(NC3CCCC3)cc2)cc1C(F)(F)F]

The title compound was synthesized in similar fashion as Example S12. ¹H NMR (400 MHz, METHANOL-d₄) δ 1.57-1.74 (m, 5H), 1.77-2.03 (m, 6H), 2.12-2.25 (m, 1 H), 2.40 (s, 3H), 3.09-3.17 (m, 1H), 3.18-3.27 (m, 1H), 3.55-3.65 (m, 1H), 3.93 (quin, J=6.97 Hz, 1H), 4.10 (br d, J=13.69 Hz, 1H), 4.28 (d, J=13.69 Hz, 1H), 5.55 (br d, J=5.87 Hz, 1H), 7.23-7.31 (m, 3H), 7.31-7.40 (m, 5H), 7.44 (br d, J=7.09 Hz, 1H), 7.65 (d, J=8.56 Hz, 2H), 7.74 (s, 1H). LC-MS: (ES) m/z 622.1 (M+Na).

### Example S44: Synthesis of (2R,3S)-2-(4-(cyclopentyl(1,7-naphthyridin-8-yl)amino)phenyl)-N-(4-methyl-3-(trifluoromethyl)phenyl)piperidine-3-carboxamide (Compound No. 168)

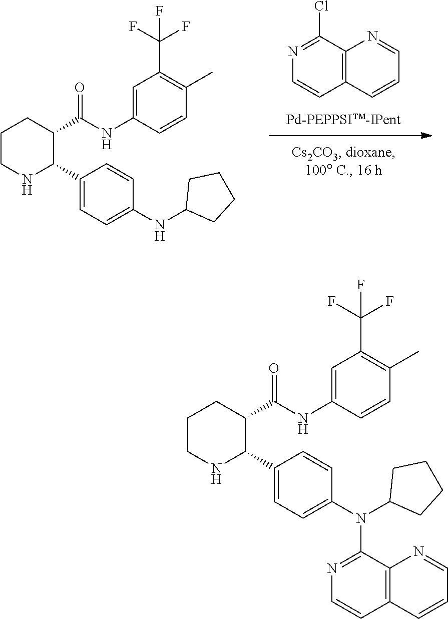 [SMILES: Cc1ccc(NC(=O)[C@H]2CCCN[C@H]2c2ccc(NC3CCCC3)cc2)cc1C(F)(F)F ; Clc1nccc2cccnc12 ; Cc1ccc(NC(=O)[C@H]2CCCN[C@H]2c2ccc(N(c3nccc4cccnc34)C3CCCC3)cc2)cc1C(F)(F)F]

(2R,3S)-2-[4-(cyclopentylamino)phenyl]-N-[4-methyl-3-(trifluoromethyl)phenyl]-piperidine-3-carboxamide (150.00 mg, 286.18 μmol) and 8-chloro-1,7-naphthyridine (70.65 mg, 429.27 mol) were dissolved in dioxane (6 mL), Pd-PEPPSI™—IPent (22.69 mg, 28.62 mol) and Cs₂CO₃ (279.73 mg, 858.54 mol) were added to the mixture. The mixture was stirred at 100° C. under N₂ for 16 h. After cooled to 25° C., the reaction mixture was filtered, the filtrate was evaporated under vacuum to give crude product, which was purified by prep-HPLC (column: Agela ASB 150*25 mm*5 um; mobile phase: [water (0.05% HCl)-ACN]; B %: 30%-60%, 8 min). The result product from prep-HPLC was purified by prep-TLC (SiO₂, DCM:MeOH=10:1) to give (2R,3S)-2-[4-[cyclopentyl(1,7-naphthyridin-8-yl)amino]phenyl]-N-[4-meth yl-3-(trifluoromethyl)phenyl]piperidine-3-carboxamide (6 mg, 8.95 mol, 3.13% yield, 91% purity, HCl) as a white solid. ¹H NMR (400 MHz, CDCl₃) δ 0.73-0.85 (5H, m), 1.13-1.34 (12H, m), 1.51-1.68 (20H, m), 1.79-1.89 (3H, m), 1.89-2.02 (4H, m), 2.16-2.24 (2H, m), 2.33 (3 H, s), 2.80-2.91 (2H, m), 3.36 (1H, br d, J=10.76 Hz), 3.92 (1H, d, J=2.45 Hz), 4.91 (1 H, br s), 6.86-6.93 (2H, m), 6.96 (1H, d, J=5.38 Hz), 6.99-7.07 (2H, m), 7.10 (2H, br d, J=8.31 Hz), 7.45 (1H, br d, J=8.07 Hz), 7.59 (1 H, s) 7.74 (1H, d, J=7.09 Hz), 7.98 (1H, br d, J=2.69 Hz), 8.09 (1H, d, J=5.62 Hz), 10.75 (1 H, s). LCMS: m/z 574.3 (M+H⁺).

### Example S45: Synthesis of (2R,3S)-2-(4-(cyclopentyl(isoquinolin-1-yl)amino)phenyl)-N-(4-methyl-3-(trifle oromethyl)phenyl)piperidine-3-carboxamide (Compound No. 175)

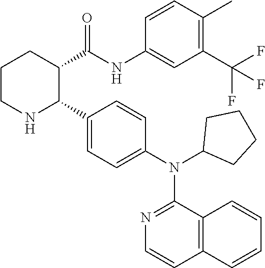 [SMILES: Cc1ccc(NC(=O)[C@H]2CCCN[C@H]2c2ccc(N(c3nccc4ccccc34)C3CCCC3)cc2)cc1C(F)(F)F]

The title compound was synthesized in similar fashion as Example S46. ¹H NMR (400 MHz, METHANOL-d₄) δ 1.43-1.73 (1H, m) 1.43-1.70 (6H, m) 1.85-2.03 (1H, m) 2.13 (2 H, br s) 2.19-2.32 (3H, m) 2.41 (3H, d, J=1.22 Hz) 3.23-3.28 (2H, m) 3.65 (1H, br d, J=11.25 Hz) 4.61 (1 H, quin, J=7.40 Hz) 4.75 (1H, d, J=3.18 Hz) 6.86 (1H, t, J=7.83 Hz) 7.28 (1H, d, J=8.31 Hz) 7.38-7.45 (3H, m) 7.47-7.55 (2H, m) 7.60 (3H, d, J=8.07 Hz) 7.81-7.93 (3H, m). LC-MS: (ES) m/z 573.3 (M+H⁺).

### Example S46: Synthesis of (2R,3S)-2-(4-(cyclopentyl(quinazolin-4-yl)amino)phenyl)-N-(4-methyl-3-(trifluor omethyl)phenyl)piperidine-3-carboxamide (Compound No. 177)

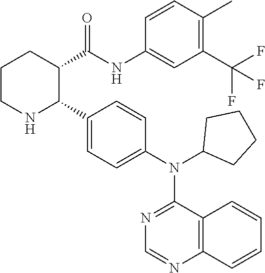 [SMILES: Cc1ccc(NC(=O)[C@H]2CCCN[C@H]2c2ccc(N(c3ncnc4ccccc34)C3CCCC3)cc2)cc1C(F)(F)F]

The title compound was synthesized in similar fashion as Example S46. ¹H NMR (400 MHz, CDCl₃) δ 1.38-1.50 (m, 2H), 1.58 (br d, J=4.02 Hz, 4H), 1.72 (br d, J=12.55 Hz, 2H), 1.98-2.09 (m, 3H), 2.32 (br d, J=12.30 Hz, 1H), 2.40 (s, 3H), 2.91-3.05 (m, 2 H), 3.49 (br d, J=10.79 Hz, 1H), 4.09 (d, J=2.76 Hz, 1H), 5.17-5.29 (m, 1H), 6.56-6.62 (m, 1H), 6.65-6.72 (m, 1H), 7.12 (br d, J=8.28 Hz, 3H), 7.34-7.42 (m, 3H), 7.52 (br d, J=8.28 Hz, 1H), 7.65-7.74 (m, 2H), 8.75 (s, 1H), 10.73 (s, 1H). LC-MS: (ES) m/z 574.2 (M+H⁺).

### Example 547: Synthesis of (2R,3S)-2-(4-(cyclopentyl(phthalazin-1-yl)amino)phenyl)-N-(4-methyl-3-(trifluo romethyl)phenyl)piperidine-3-carboxamide (Compound No. 179)

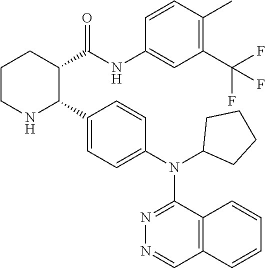 [SMILES: Cc1ccc(NC(=O)[C@H]2CCCN[C@H]2c2ccc(N(c3nncc4ccccc34)C3CCCC3)cc2)cc1C(F)(F)F]

The title compound was synthesized in similar fashion as Example S46. ¹HNMR (400 MHz, CDCl₃) δ 1.22-1.30 (2H, m), 1.80-1.98 (7H, m), 2.04 (2 H, br s), 2.25 (1 H, br d, J=11.74 Hz), 2.39 (3 H, br s), 2.90 (2 H, br s), 3.42 (1H, br d, J=11.98 Hz), 3.97 (1 H, br s), 4.92 (1 H, br s), 6.99 (2H, br d, J=8.07 Hz), 7.10 (1H, br d, J=8.31 Hz), 7.15-7.23 (3H, m), 7.49 (3 H, br dd, J=18.83, 7.83 Hz), 7.60 (1 H, br s), 7.71 (1H, br d, J=7.83 Hz), 9.11 (1 H, s), 10.73 (1 H, br s). LCMS: m/z 574.3 (M+H⁺).

### Example S48: Synthesis of (2R,3S)-2-(4-(cyclopentyl(thiazolo[4,5-c]pyridin-4-yl)amino)phenyl)-N-(4-methy l-3-(trifluoromethyl)phenyl)piperidine-3-carboxamide (Compound No. 169)

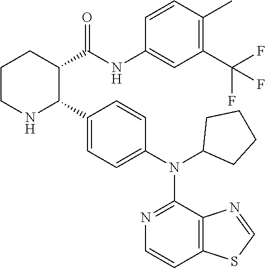 [SMILES: Cc1ccc(NC(=O)[C@H]2CCCN[C@H]2c2ccc(N(c3nccc4scnc34)C3CCCC3)cc2)cc1C(F)(F)F]

The title compound was synthesized in similar fashion as S46. ¹H NMR (400 MHz, CDCl₃) δ 0.65-0.94 (m, 1H), 1.41-1.62 (m, 6H), 1.66-2.05 (m, 8H), 2.22-2.35 (m, 1H), 2.40 (s, 3H), 2.91-3.11 (m, 2H), 3.48 (br d, J=11.0 Hz, 1H), 4.08 (br s, 1H), 5.16-5.31 (m, 1H), 7.08 (d, J=8.3 Hz, 2H), 7.14 (d, J=8.3 Hz, 1H), 7.23 (d, J=5.5 Hz, 1 H), 7.30 (d, J=8.3 Hz, 2H), 7.55 (dd, J=8.3, 1.5 Hz, 1H), 7.66 (s, 1H), 8.09 (d, J=5.5 Hz, 1 H), 8.21 (s, 1H), 10.76 (br s, 1H). LCMS: m/z 580.3 (M+H⁺).

### Example S49: Synthesis of (2R,3S)-2-(4-(cyclopentyl(pyrido[3,4-b]pyrazin-5-yl)amino)phenyl)-N-(4-methy l-3-(trifluoromethyl)phenyl)piperidine-3-carboxamide (Compound No. 172)

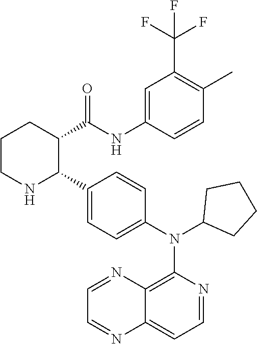 [SMILES: Cc1ccc(NC(=O)[C@H]2CCCN[C@H]2c2ccc(N(c3nccc4nccnc34)C3CCCC3)cc2)cc1C(F)(F)F]

The title compound was synthesized in similar fashion as S46. ¹H NMR (400 MHz, METHANOL-d₄) δ 1.36-1.56 (m, 2H), 1.58-1.70 (m, 4H), 1.86-1.94 (m, 1H), 2.02-2.17 (m, 2H), 2.19-2.25 (m, 2H), 2.40 (s, 3H), 3.19 (br s, 1H), 3.60 (br d, J=12.47 Hz, 1H), 4.33-4.44 (m, 1H), 4.47-4.54 (m, 2H), 4.72 (dd, J=4.03, 2.08 Hz, 1H), 6.74 (dd, J=8.80, 7.34 Hz, 2H), 6.92 (d, J=6.36 Hz, 1H), 7.27 (d, J=8.31 Hz, 1H), 7.37 (d, J=8.80 Hz, 2H), 7.47-7.58 (m, 1H), 7.67-7.72 (m, 1H), 7.72 (d, J=2.93 Hz, 1H), 7.93 (br d, J=8.31 Hz, 1H), 10.14 (br d, J=2.93 Hz, 1H). LC-MS: (ES) m/z 575.4 (M+H⁺).

### Example S50: Synthesis of (2R,3S)-2-(4-(cyclopentyl(thieno[2,3-c]pyridin-7-yl)amino)phenyl)-N-(4-methyl-3-(trifluoromethyl)phenyl)piperidine-3-carboxamide (Compound No. 173)

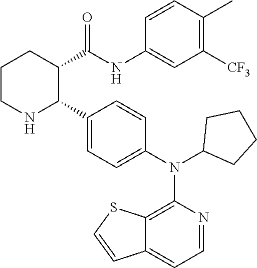 [SMILES: Cc1ccc(NC(=O)[C@H]2CCCN[C@H]2c2ccc(N(c3nccc4ccsc34)C3CCCC3)cc2)cc1C(F)(F)F]

The title compound was synthesized in similar fashion as S46. ¹H NMR (400 MHz, CDCl₃) δ 1.28-1.53 (m, 6H), 1.72 (br d, J=13.8 Hz, 2H), 1.85-2.08 (m, 5H), 2.27-2.43 (m, 5H), 2.91-3.08 (m, 2H), 3.50 (br d, J=10.0 Hz, 1H), 4.12 (d, J=2.5 Hz, 1H), 5.10-5.26 (m, 1H), 7.01 (s, 2H), 7.05 (d, J=5.5 Hz, 1H), 7.14 (d, J=8.5 Hz, 1H), 7.23 (d, J=8.3 Hz, 2H), 7.36 (d, J=8.3 Hz, 2H), 7.56 (br d, J=8.0 Hz, 1H), 7.71 (s, 1H), 8.07 (d, J=5.5 Hz, 1H), 10.77 (s, 1H). LC-MS: (ES) m/z 579.23 (M+H⁺).

### Example S51: Synthesis of (2R,3S)-2-(4-(cyclopentyl(phthalazin-1-yl)amino)phenyl)-N-(4-methyl-3-(trifleo romethyl)phenyl)piperidine-3-carboxamide (Compound No. 179)

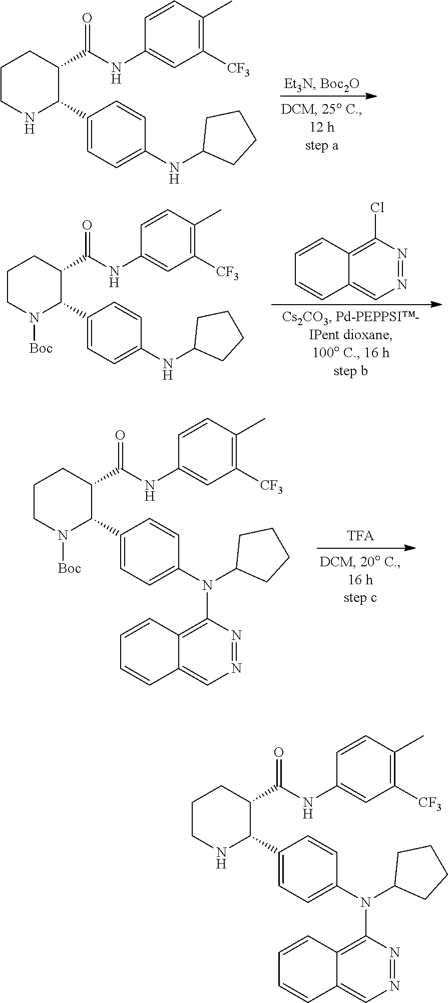

Step a) To a mixture of (2R,3S)-2-[4-(cyclopentylamino)phenyl]-N-[4-methyl-3-(trifluoromethyl)phenyl]piperidine-3-carboxamide (1 g, 1.91 mmol) and Et₃N (386.12 mg, 3.82 mmol, 531.11 μL) in DCM (15 mL) was added Boc₂O (416.39 mg, 1.91 mmol, 438.31 L) in one portion at 25° C. The mixture was stirred at 25° C. for 12 hours. A light brown solution was noted. The reaction mixture was quenched with H₂O (20 mL) and extracted with EtOAc (2×30 mL). The combined organic layers were dried, filtered and concentrated in vacuo to give the residue. The residue was purified by flash silica gel chromatography (ISCO®; 4 g SepaFlash® Silica Flash Column, elution of 0˜30% Ethyl acetate/Petroleum ether gradient@ 25 mL/min) to give a target product tert-butyl(2R,3S)-2-[4-(cyclopentylamino)phenyl]-3-[[4-methyl-3-(trifluoro methyl)phenyl]carbamoyl]piperidine-1-carboxylate (540 mg, 940.19 mol, 49.28% yield, 95% purity) as white solid. ¹H NMR (400 MHz, CDCl₃) δ 1.36-1.45 (m, 2H), 1.48 (s, 9H), 1.46-1.51 (m, 1H), 1.52-1.75 (m, 5H), 1.83 (br d, J=13.30 Hz, 1H), 1.90-1.99 (m, 3H), 2.08-2.23 (m, 1H), 2.39 (d, J=1.51 Hz, 3 H), 2.85-3.03 (m, 2H), 3.57-3.75 (m, 2H), 3.95 (br d, J=14.05 Hz, 1H), 5.79 (br d, J=5.27 Hz, 1H), 6.43-6.50 (m, 2H), 7.08-7.21 (m, 3H), 7.38 (dd, J=8.28, 2.26 Hz, 1H), 7.59 (s, 1H), 8.00 (br s, 1H). LC-MS: (ES) m/z 546.3 (M+H⁺).

Step b) Tert-butyl(2R,3S)-2-[4-(cyclopentylamino)phenyl]-3-[[4-methyl-3-(trifluoromethyl)phenyl]carbamoyl]piperidine-1-carboxylate (150 mg, 274.91 mol) and 1-chlorophthalazine (67.87 mg, 412.36 mol) were dissolved in dioxane (6 mL), Cs₂CO₃ (268.71 mg, 824.73 mol) and Pd-PEPPSI™-IPent (21.80 mg, 27.49 μmol) were added to the mixture. The mixture was stirred at 100° C. under N₂ for 16 h. The mixture was filtered, the filtrate was evaporated under vacuum to give crude product. The crude product was purified by column chromatography (SiO₂, Petroleum ether/Ethyl acetate=100/0 to 1:1) and prep-TLC (SiO₂, Petroleum ether:Ethyl acetate=1:1) to give the target product tert-butyl(2R,3S)-2-[4-[cyclopentyl(phthalazin-1-yl)amino]phenyl]-3-[[4-methyl-3-(trifluoromethyl)phenyl]carbamoyl]piperidine-1-carboxylate (12 mg, 17.81 mol, 6.48% yield, 100% purity) as a yellow solid. ¹H NMR (400 MHz, CDCl₃) δ 1.19-1.33 (6H, m) 1.56 (9 H, br s) 1.77-1.95 (7H, m) 2.14-2.23 (1H, m) 2.39 (3 H, s) 2.82 (1 H, brt, J=13.08 Hz) 2.89-3.00 (1H, m) 3.93 (1H, br d, J=12.23 Hz) 4.84 (1 H, br s) 5.28 (1 H, s) 5.78 (1 H, br s) 6.92 (2H, br d, J=8.31 Hz) 7.11 (1H, br d, J=8.07 Hz) 7.25 (2 H, s) 7.31 (1 H, br t, J=7.83 Hz) 7.45 (2H, br d, J=8.31 Hz) 7.59 (1H, t, J=7.46 Hz) 7.64 (1 H, s) 7.76 (1H, d, J=8.07 Hz) 8.17 (1H, br d, J=13.94 Hz) 9.15 (1 H, s). LC-MS: (ES) m/z 674.3 (M+H⁺).

Step c) To a mixture of tert-butyl(2R,3S)-2-[4-[cyclopentyl(phthalazin-1-yl)amino]-phenyl]-3-[[4-methyl-3-(trifluoromethyl)phenyl]carbamoyl]piperidine-1-carboxylate (8 mg, 11.87 mol) in DCM (2 mL) was added TFA (154.00 mg, 1.35 mmol, 0.1 mL). The mixture was stirred at 25° C. for 1 h. A light yellow solution was noted. The reaction mixture was concentrated in vacuo to give the residue. The residue was diluted with DCM (10 mL) and alkalified to pH=8-9 by addition of saturated NaHCO₃ solution, washed with brine (2×5 mL), dried, filtered and concentrated in vacuo to give the crude product. The crude product was washed with mixed solvents (0.5 mL, Petroleum ether/EtOAc=10/1) and dried in vacuo to give the desired product to give the target product (2R,3S)-2-[4-[cyclopentyl(phthalazin-1-yl)amino]phenyl]-N-[4-methyl-3-(trifluoromethyl)phenyl]piperidine-3-carboxamide (4.5 mg, 7.77 mol, 65.41% yield, 99% purity) as light yellow solid. ¹H NMR (400 MHz, CDCl₃) δ 1.69 (br s, 2H), 1.85-2.13 (m, 8H), 2.17-2.32 (m, 2H), 2.41 (s, 3H), 2.86-2.98 (m, 2H), 3.44 (br d, J=11.49 Hz, 1H), 3.99 (d, J=2.69 Hz, 1H), 4.93 (br s, 1H), 5.24-5.49 (m, 1H), 7.01 (d, J=8.31 Hz, 2H), 7.12 (d, J=8.07 Hz, 1 H), 7.18-7.25 (m, 3H), 7.28-7.37 (m, 1H), 7.39-7.55 (m, 3H), 7.63 (s, 1H), 7.73 (d, J=8.07 Hz, 1H), 9.12 (s, 1H), 10.75 (s, 1H). LC-MS: (ES) m/z 574.3 (M+H⁺).

### Example S52: Synthesis of (2R,3R)-2-(4-(cyclopentyl(1,7-naphthyridin-8-yl)amino)phenyl)-N-(4-methyl-3-(trifluoromethyl)phenyl)piperidine-3-carboxamide and (2R,3R)-2-(4-(cyclopentyl(1,7-naphth yridin-8-yl)amino)phenyl)-N-(4-methyl-3-(trifluoromethyl)phenyl)piperidine-3-carboxamide (Compound Nos. 168 and 171)

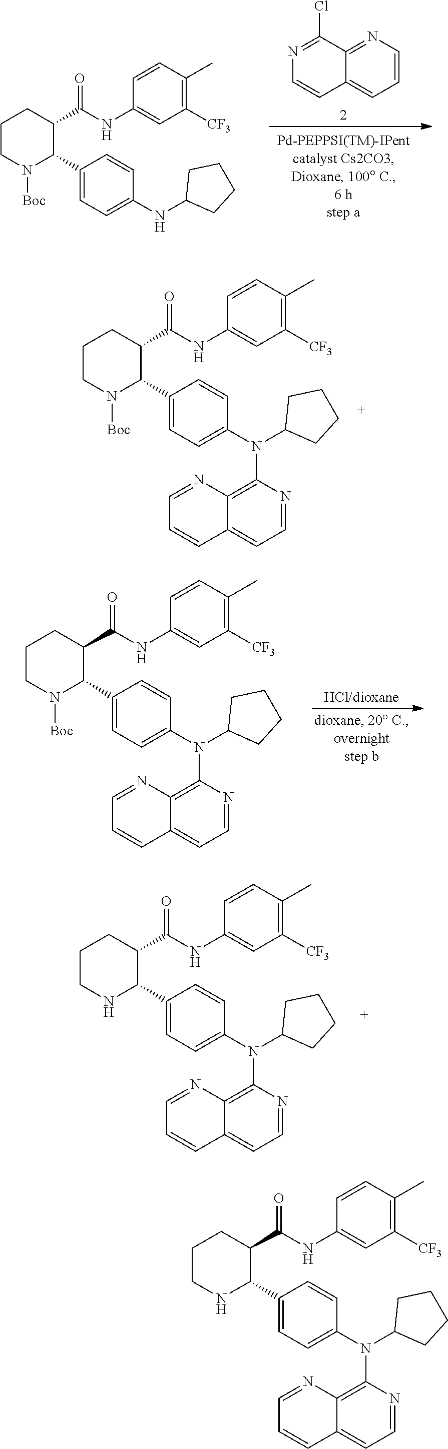

Step a) A mixture of tert-butyl(2R,3S)-2-[4-(cyclopentylamino)phenyl]-3-[[4-methyl-3-(trifluoromethyl)phenyl]carbamoyl]piperidine-1-carboxylate (200 mg, 366.55 mol), 8-chloro-1,7-naphthyridine (75 mg, 455.67 mol), Pd-PEPPSI™-IPent catalyst (29.09 mg, 36.66 mol) and Cs₂CO₃ (358.28 mg, 1.10 mmol) in dioxane (4 mL) was stirred at 100° C. for 6 h. A brown suspension was noted. The reaction mixture was quenched with H₂O (5 mL) and extracted with EtOAc (3×5 mL). The combined organic layers were washed with brine (3×3 mL), dried and concentrated in vacuo to give the crude product. The crude product was purified by prep-TLC. The desired compounds tert-butyl(2R,3S)-2-[4-[cyclopentyl(1,7-naphthyridin-8-yl)amino] phenyl]-3-[[4-methyl-3-(trifluoromethyl)phenyl]carbamoyl]piperidine-1-carboxylate (20 mg, 29.68 mol, 6.67% yield, 100% purity) LC-MS: (ES) m/z 674.4 (M+H⁺) and tert-butyl(2R,3R)-2-[4-[cyclopentyl(1,7-naphthyridin-8-yl)amino]phenyl]-3-[[4-methyl-3-(trifleoromethyl)phenyl]carbamoyl]piperidine-1-carboxylate (20 mg, 29.68 mol, 6.67% yield, 100% purity) were obtained as light yellow gum. LC-MS: (ES) m/z 674.4 (M+H⁺).

Step b) To a solution of tert-butyl(2R,3S)-2-[4-[cyclopentyl(1,7-naphthyridin-8-yl)amino]phenyl]-3-[[4-methyl-3-(trifluoromethyl)phenyl]carbamoyl]piperidine-1-carboxylate (20 mg, 29.68 mol) in DCM (0.5 mL) was added HCl/dioxane (4 M, 74.21 μL). Then the mixture was stirred at 25° C. for 1 h. The reaction mixture was concentrated in vauco to give the crude product. The crude was washed with MTBE (3*1 mL) and dried in vacuo to give the desired product (HCl salt). The product was dissolved with H₂O (3 mL) and alkalified to pH=9˜10, then the mixture was extracted with DCM (3*5 mL). The combined organic layers were dried, filtered and concentrated in vacuo to give the desired product as light yellow gum, which was lyophilized. The desired compound (2R,3S)-2-[4-[cyclopentyl(1,7-naphthyridin-8-yl)amino]phenyl]-N-[4-methyl-3-(trifluoromethyl)phenyl]piperidine-3-carboxamide (8 mg, 13.95 mol, 46.98% yield, 100% purity) was obtained as yellow solid. ¹H NMR (400 MHz, CDCl₃) δ 1.63-1.71 (m, 2H), 1.73-2.11 (m, 9H), 2.27 (br d, J=12.55 Hz, 1H), 2.41 (d, J=1.51 Hz, 3H), 2.85-3.02 (m, 2 H), 3.43 (br d, J=11.29 Hz, 1H), 4.00 (d, J=2.76 Hz, 1H), 4.92-5.08 (m, 1H), 6.99 (d, J=8.53 Hz, 2H), 7.04 (d, J=5.77 Hz, 1H), 7.10 (dd, J=8.28, 4.02 Hz, 1H), 7.13 (d, J=8.03 Hz, 1H), 7.18 (d, J=8.28 Hz, 2H), 7.53 (dd, J=8.28, 2.01 Hz, 1H), 7.67 (d, J=1.76 Hz, 1H), 7.82 (dd, J=8.28, 1.76 Hz, 1H), 8.06 (dd, J=4.27, 1.76 Hz, 1H), 8.17 (d, J=5.77 Hz, 1H), 10.83 (s, 1H). LC-MS: (ES) m/z 574.4 (M+H⁺).

Step b) To a solution of tert-butyl(2R,3R)-2-[4-[cyclopentyl(1,7-naphthyridin-8-yl)amino]phenyl]-3-[[4-methyl-3-(trifluoromethyl)phenyl]carbamoyl]piperidine-1-carboxylate (20.00 mg, 29.68 mol) in DCM (0.5 mL) was added HCl/dioxane (4 M, 0.1 mL). Then the mixture was stirred at 25° C. for 1 h. The reaction mixture was concentrated in vauco to give the crude product. The crude product was washed with MTBE (3*1 mL) and dried in vacuo to give the desired product (HCl salt). The product was dissolved with H₂O (3 mL) and alkalified to pH=9˜10, then the mixture was extracted with DCM (3*5 mL). The combined organic layers were dried, filtered and concentrated in vacuo to give the crude product. The product was further purified by prep-TLC (DCM/MeOH=10/1). The desired compound (2R,3R)-2-[4-[cyclopentyl(1,7-naphthyridin-8-yl)amin o]phenyl]-N-[4-methyl-3-(trifluoromethyl)phenyl]piperidine-3-carboxamide (6.8 mg, 11.62 mol, 39.14% yield, 98% purity) was obtained as yellow solid. ¹H NMR (400 MHz, CDCl₃) δ 1.61 (br s, 2H), 1.68-1.81 (m, 2H), 1.88-2.05 (m, 4H), 2.15-2.37 (m, 4H), 2.42 (s, 3H), 2.56 (br t, J=9.29 Hz, 1H), 2.85 (td, J=11.55, 3.55 Hz, 1H), 3.20 (br d, J=11.49 Hz, 1H), 3.90 (d, J=10.03 Hz, 1 H), 4.91 (br d, J=6.85 Hz, 1H), 6.94 (d, J=8.31 Hz, 2H), 7.03 (dd, J=8.19, 4.03 Hz, 1H), 7.07-7.16 (m, 2H), 7.29-7.40 (m, 4H), 7.57 (s, 1H), 7.85 (d, J=7.58 Hz, 1H), 8.01 (br d, J=3.42 Hz, 1H), 8.19 (d, J=5.62 Hz, 1H). LCMS: m/z 574.4 (M+H⁺).

### Example S53: Synthesis of (2R,3S)-2-(4-(cyclopentyl(pyrido[3,2-d]pyrimidin-4-yl)amino)-phenyl)-N-(4-methyl-3-(trifluoromethyl)phenyl)piperidine-3-carboxamide and (2R,3R)-2-(4-(cyclopentyl-(pyrido[3,2-d]pyrimidin-4-yl)amino)phenyl)-N-(4-methyl-3-(trifluoromethyl)-phenyl)piperidine-3-carboxamide (Compound Nos. 174 and 176)

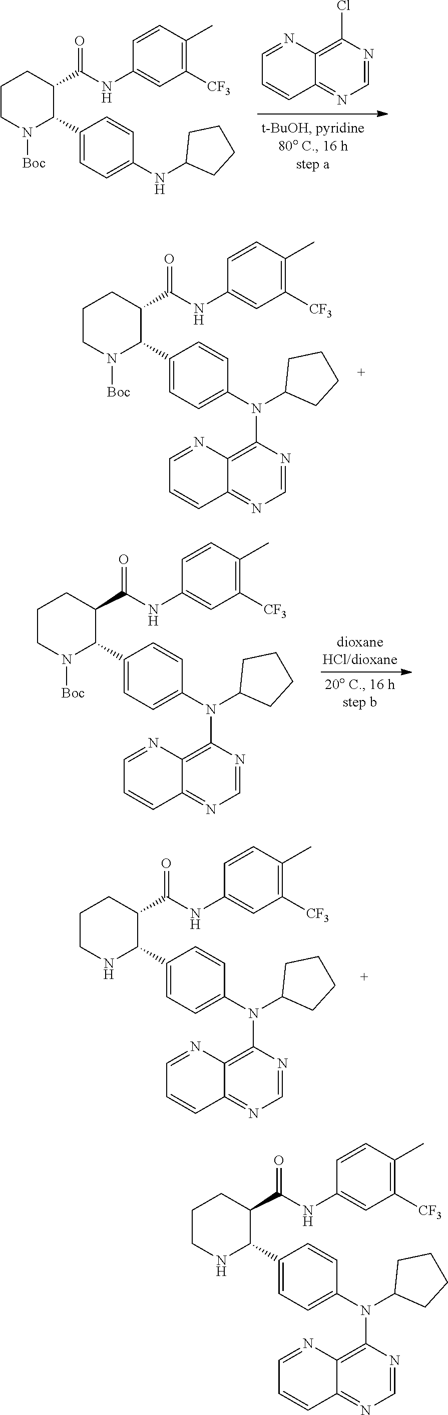

Step a) To a solution of tert-butyl (2R,3S)-2-[4-(cyclopentylamino)phenyl]-3-[[4-methyl-3-(trifluoromethyl)phenyl]carbamoyl]piperidine-1-carboxylate (120 mg, 219.93 mol) in tert-butyl alcohol (1 mL) was added pyridine (17.40 mg, 219.93 mol, 17.75 μL) and 4-chloropyrido[3,2-d]pyrimidine (33.14 mg, 200.13 mol). The mixture was stirred at 80° C. for 16 hr. The reaction mixture was filtered and concentrated under reduced pressure to give a residue. The residue was purified by prep-TLC to give tert-butyl(2R,3S)-2-[4-[cyclopentyl(pyrido-[3,2-d]pyrimidin-4-yl)amino]phenyl]-3-[[4-methyl-3-(trifluoromethyl)phenyl]carbamoyl]piperidine-1-carboxylate (12 mg, 16.36 mol, 7.44% yield, 92% purity) as a light yellow solid (¹H NMR (400 MHz, CDCl₃) δ 1.49 (s, 9H), 1.56-1.72 (m, 4H), 1.92 (br s, 3H), 1.98-2.06 (m, 3H), 2.07-2.14 (m, 1H), 2.15-2.27 (m, 1 H), 2.37-2.47 (m, 4H), 2.93-3.01 (m, 1H), 3.02-3.08 (m, 1H), 4.06 (br d, J=11.49 Hz, 1 H), 5.40-5.53 (m, 1H), 5.91 (br d, J=4.40 Hz, 1H), 7.03 (d, J=8.31 Hz, 2H), 7.15 (d, J=8.31 Hz, 1H), 7.27-7.31 (m, 1H), 7.39 (d, J=8.31 Hz, 2H), 7.50 (br d, J=8.31 Hz, 1H), 7.67 (s, 1H), 7.97 (dd, J=8.44, 1.59 Hz, 1H), 8.02 (dd, J=3.91, 1.47 Hz, 1H), 8.67 (s, 1H). LC-MS: (ES) m/z 675.3 (M+H⁺)) and tert-butyl(2R,3R)-2-[4-[cyclopentyl (pyrido[3,2-d]pyrimidin-4-yl)amino]phenyl]-3-[[4-methyl-3-(trifluoromethyl)phenyl]carbamoyl]piperidine-1-carboxylate (53 mg, 76.98 umol, 35.00% yield, 98% purity) as a yellow solid (¹H NMR (400 MHz, CDCl₃) δ 1.52 (s, 9H), 1.56-1.73 (m, 8H), 1.81-1.94 (m, 1H), 2.09 (br d, J=5.87 Hz, 2H), 2.41 (br s, 1H), 2.45 (s, 3H), 2.95 (td, J=12.90, 3.30 Hz, 1H), 3.39 (br s, 1H), 4.03-4.13 (m, 1H), 5.51-5.62 (m, 1H), 5.98 (br s, 1H), 7.13-7.19 (m, 2H), 7.25 (d, J=8.31 Hz, 3H), 7.42 (dd, J=8.44, 4.03 Hz, 1 H), 7.80-7.89 (m, 2H), 8.03 (dd, J=8.44, 1.34 Hz, 1H), 8.23 (dd, J=3.91, 1.47 Hz, 1H), 8.70 (s, 1H), 8.95 (br s, 1H). LC-MS: (ES) m/z 675.3 (M+H⁺)).

Step b) To a solution of tert-butyl(2R,3S)-2-[4-[cyclopentyl(pyrido[3,2-d]pyrimidin-4-yl)amino]phenyl]-3-[[4-methyl-3-(trifluoromethyl)phenyl]carbamoyl]piperidine-1-carboxylate (12 mg, 17.78 mol) in dioxane (1 mL) was added HCl/dioxane (4 M, 44.46 μL). The mixture was stirred at 20° C. for 16 hr. The reaction mixture was filtered and concentrated under reduced pressure to give a residue. Then the residue was alkalized with aq. NaHCO₃ (3 ml), then extracted with DCM 50 mL (25 mL*2). The combined organic layers were dried over Na₂SO₄, filtered and concentrated under reduced pressure to give the product. The residue was purified by prep-HPLC (basic condition, column: Xtimate C18 10 μm 250 mm*50 mm; mobile phase: [water (0.04% NH₃H₂O+10 mM NH₄HCO₃)-ACN]; B %: 65%-95%, 8 min to give (2R,3S)-2-[4-[cyclopentyl(pyrido[3,2-d]pyrimidin-4-yl)amino]phenyl]-N-[4-methyl-3-(trifluoromethyl)phenyl]-piperidine-3-carboxamide (5 mg, 8.61 mol, 48.44% yield, 99% purity) as a light yellow solid. ¹H NMR (400 MHz, CDCl₃) δ 1.44 (br d, J=6.60 Hz, 2H), 1.52-1.62 (m, 5H), 1.90-2.09 (m, 4H), 2.32 (br d, J=12.23 Hz, 1H), 2.39 (s, 3H), 2.97 (br t, J=11.74 Hz, 1H), 3.03 (br s, 1H), 3.49 (br d, J=11.49 Hz, 1H), 4.10 (br d, J=2.20 Hz, 1 H), 5.38-5.51 (m, 1H), 7.07 (br d, J=8.07 Hz, 2H), 7.13 (br d, J=8.31 Hz, 1H), 7.21 (dd, J=8.19, 4.28 Hz, 1H), 7.32 (br d, J=8.31 Hz, 2H), 7.55 (br d, J=8.31 Hz, 1H), 7.72 (s, 1H), 7.84-7.96 (m, 2H), 8.66 (s, 1H), 10.83 (s, 1H). LC-MS: (ES) m/z 575.3 (M+H⁺).

Step b) To a solution of tert-butyl (2R,3R)-2-[4-[cyclopentyl(pyrido[3,2-d]pyrimidin-4-yl)amino]phenyl]-3-[[4-methyl-3-(trifluoromethyl)phenyl]carbamoyl]piperidine-1-carboxylate (50 mg, 74.10 mol) in dioxane (1 mL) was added HCl/dioxane (4 M, 185.25 μL). The mixture was stirred at 20° C. for 16 hr. No further monitoring. The reaction mixture was filtered and concentrated under reduced pressure to give a residue. Then the residue was alkalized with aq. NaHCO₃ (3 ml), then extracted with DCM 50 mL (25 mL*2). The combined organic layers were dried over Na₂SO₄, filtered and concentrated under reduced pressure to give the residue. The residue was purified by prep-HPLC (basic condition, column: Xtimate C18 10 μm 250 mm*50 mm; mobile phase: [water (0.04% NH₃H₂O+10 mM NH₄HCO₃)-ACN]; B %: 52%-82%, 8 min) to give (2R,3R)-2-[4-[cyclopentyl(pyrido[3,2-d]pyrimidin-4-yl)amino]phenyl]-N-[4-methyl-3-(trifluoromethyl)phenyl]piperidine-3-carboxamide (22 mg, 37.90 mol, 51.15% yield, 99% purity) as a light yellow solid. ¹H NMR (400 MHz, CDCl₃) δ 1.25-1.42 (m, 4H), 1.51 (br d, J=5.62 Hz, 2H), 1.70 (q, J=12.88 Hz, 2H), 1.95-2.05 (m, 3H), 2.06-2.15 (m, 1H), 2.41 (s, 3H), 2.43-2.50 (m, 1H), 2.87-2.96 (m, 1H), 3.23 (br d, J=11.49 Hz, 1H), 3.97 (d, J=9.54 Hz, 1H), 5.43-5.55 (m, 1H), 7.05 (d, J=8.31 Hz, 2H), 7.14 (dt, J=8.19, 3.97 Hz, 2 H), 7.25 (br s, 1H), 7.40-7.49 (m, 3H), 7.55-7.61 (m, 1H), 7.58 (s, 1H), 7.85 (br d, J=2.45 Hz, 1H), 7.93 (dd, J=8.44, 1.34 Hz, 1H), 8.61 (s, 1H). LC-MS: (ES) m/z 575.3 (M+H⁺).

### Example S54: Synthesis of (2R,3S)-2-(4-(cyclopentyl(pyrido[3,4-d]pyrimidin-4-yl)amino)phenyl)-N-(4-methyl-3-(trifluoromethyl)phenyl)piperidine-3-carboxamide (Compound No. 178)

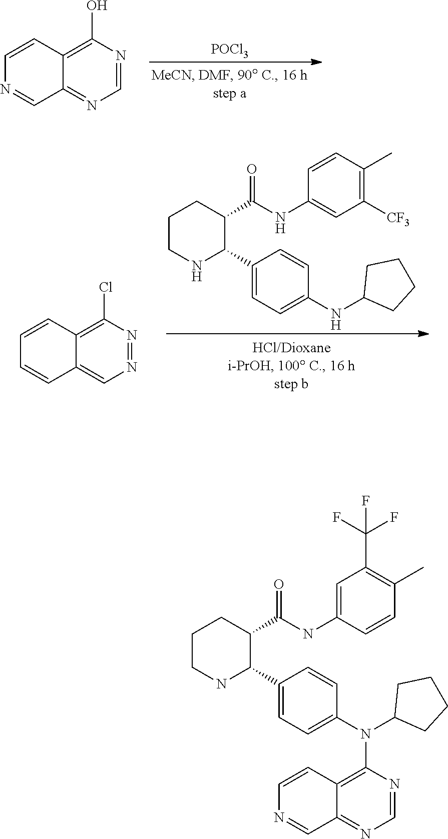 [SMILES: Oc1ncnc2cnccc12 ; "POCl3" ; Clc1nncc2ccccc12 ; Cc1ccc(NC(=O)[C@H]2CCCN[C@H]2c2ccc(NC3CCCC3)cc2)cc1C(F)(F)F ; Cc1ccc(NC(=O)[C@H]2CCCN[C@H]2c2ccc(N(c3ncnc4cnccc34)C3CCCC3)cc2)cc1C(F)(F)F]

Step a) To a solution of pyrido[3,4-d]pyrimidin-4-ol (50 mg, 339.83 mol) in MeCN (1 mL) was added DMF (4.72 mg, 64.55 mol, 4.97 μL), then the POCl₃ (2.57 g, 16.76 mmol, 1.56 mL) was added. The mixture was stirred at 90° C. for 16 hr. The reaction mixture was concentrated under reduced pressure to remove POCl₃. The residue was diluted with aq. NaHCO₃ 10 mL and extracted with EtOAc 60 mL (30 mL*2). The combined organic layers were dried over anhydrous Na₂SO₄, filtered and concentrated under reduced pressure to give the pure product. Compound 4-chloropyrido[3,4-d]pyrimidine (170 mg) was obtained as a gray solid. ¹H NMR (400 MHz, DMSO) δ 8.01 (d, J=5.38 Hz, 1H), 8.27 (s, 1 H), 8.69 (d, J=5.38 Hz, 1H), 9.11 (s, 1H). LC-MS: (ES) m/z 166.0 (M+H⁺).

Step b) To a solution of (2R,3S)-2-[4-(cyclopentylamino)phenyl]-N-[4-methyl-3-(trifluoromethyl)phenyl]piperidine-3-carboxamide (150 mg, 336.69 mol) in isopropyl alcohol (1.2 mL) was added HCl/dioxane (4 M, 105.21 μL) and then the 4-chloropyrido[3,4-d]pyrimidine (66.90 mg, 404.02 mol) was added. The mixture was stirred at 100° C. for 16 hr. The reaction mixture was alkalized with aq. NaHCO₃ 6 mL and extracted with EtOAc 50 mL. The combined organic layers were dried over anhydrous Na₂SO₄, filtered and concentrated under reduced pressure to give a residue. The residue was purified by prep-HPLC (basic condition, column: Xtimate C18 10 μm 250 mm*50 mm; mobile phase: [water (0.04% NH₃H₂O+10 mM NH₄HCO₃)-ACN]; B %: 70%-100%, 8 min) to give (2R,3S)-2-[4-[cyclopentyl(pyrido[3,4-d]pyrimidin-4-yl)amino]phenyl]-N-[4-methyl-3-(trifluoromethyl)phenyl]piperidine-3-carboxamide (20 mg, 33.76 mol, 10.03% yield, 97% purity) as a light yellow solid. ¹H NMR (400 MHz, CDCl₃) δ 1.32-1.47 (m, 2H), 1.51-1.62 (m, 4H), 1.74 (br d, J=12.80 Hz, 2H), 1.98-2.06 (m, 3H), 2.33 (br d, J=11.80 Hz, 1H), 2.39 (br d, J=1.26 Hz, 3H), 2.95-3.05 (m, 2H), 3.52 (br d, J=11.29 Hz, 1H), 4.14 (d, J=3.01 Hz, 1H), 5.21-5.31 (m, 1H), 6.02 (d, J=6.02 Hz, 1H), 7.13-7.21 (m, 3H), 7.45 (d, J=8.28 Hz, 2H), 7.53 (d, J=2.01 Hz, 1H), 7.69 (dd, J=8.28, 1.76 Hz, 1H), 7.86 (d, J=6.02 Hz, 1H), 8.81 (s, 1H), 9.13 (s, 1H), 10.74 (s, 1H). LC-MS: (ES) m/z 575.3 (M+H⁺).

### Example S55: Synthesis of (2S,3S)-1-(2-fluoro-6-methylbenzoyl)-N-(4-methyl-3-(trifluoromethyl)-phenyl)-2-(2-oxaspiro[4.5]decan-8-yl)piperidine-3-carboxamide (Compound No. 170)

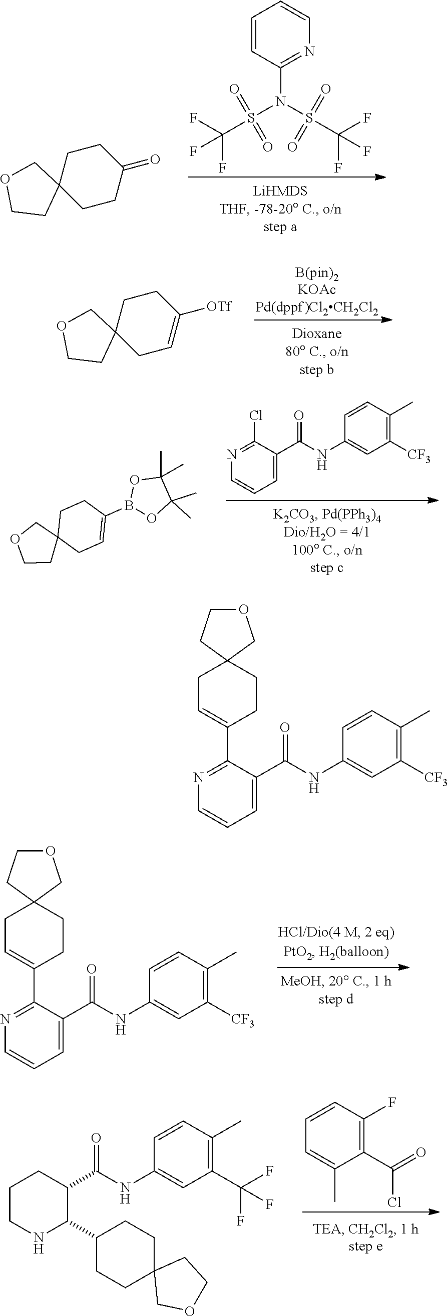

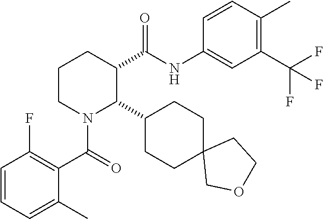

Step a) To a solution of 2-oxaspiro[4.5]decan-8-one (300 mg, 1.95 mmol, 422.39 L) in THF (12 mL) was added LiHMDS (2 M in THF/heptane) (2 M, 1.26 mL) at −78° C. After stirred for 30 min, the 1,1,1-trifluoro-N-phenyl-N-(trifluoromethylsulfonyl)methanesulfonamide (1.04 g, 2.92 mmol) in THF (6 mL) was added. The mixture was stirred at 20° C. for 15.5 hr. Saturated aqueous NaHCO₃ (20 ml) solution was added followed by dilution with EtOAc (80 ml). The organic layer was dried over Na₂SO₄, filtered and the solvent removed under reduced pressure to give the residue. The residue was purified by column chromatography (SiO₂, Petroleum ether/Ethyl acetate=15/1 to 10:1) to give 2-oxaspiro[4.5]dec-7-en-8-yltrifluoromethanesulfonate (430 mg, 1.50 mmol, 77.21% yield) as a yellow oil. ¹H NMR (400 MHz, CDCl₃) δ 1.73-1.84 (m, 4H), 2.23 (br d, J=3.18 Hz, 2H), 2.38-2.46 (m, 2H), 3.55 (d, J=1.47 Hz, 2H), 3.91 (t, J=7.09 Hz, 2H), 5.75 (t, J=4.03 Hz, 1H) LC-MS: (ES) m/z 287.05 (M+H⁺).

Step b) A mixture of 2-oxaspiro[4.5]dec-7-en-8-yl trifluoromethanesulfonate (430 mg, 1.50 mmol), KOAc (294.83 mg, 3.00 mmol) and 4,4,5,5-tetramethyl-2-(4,4,5,5-tetramethyl-1,3,2-dioxaborolan-2-yl)-1,3,2-dioxaborolane (420 mg, 1.65 mmol) in dioxane (6 mL), after 5 min, the Pd(dppf)Cl₂·CH₂Cl₂ (61.33 mg, 75.10 mol) was added. The mixture was degassed and purged with N₂ 3 times, and then the mixture was stirred at 80° C. for 15 h 55 min under N₂ atmosphere. The reaction mixture was filtered and concentrated under reduced pressure to give a residue. The residue was purified by column chromatography (SiO₂, Petroleum ether/Ethyl acetate=20/1 to 10:1). Compound 4,4,5,5-tetramethyl-2-(2-oxaspiro[4.5]dec-7-en-8-yl)-1,3,2-dioxaborolane (304 mg, 1.15 mmol, 76.61% yield) was obtained as a yellow oil. ¹H NMR (400 MHz, CDCl₃) δ 1.27 (s, 12H), 1.55-1.63 (m, 2H), 1.65-1.77 (m, 2H), 2.10-2.14 (m, 2H), 2.16-2.24 (m, 2H), 3.51 (s, 2H), 3.82-3.91 (m, 2H), 6.49-6.55 (m, 1H). LC-MS: (ES) m/z 265.2 (M+H⁺).

Step c) A mixture of 2-chloro-N-[4-methyl-3-(trifluoromethyl)phenyl]pyridine-3-carboxamide (130 mg, 413.11 mol), 4,4,5,5-tetramethyl-2-(2-oxaspiro[4.5]dec-7-en-8-yl)-1,3,2-dioxaborolane (140 mg, 529.97 mol), Pd(PPh₃)₄ (95.47 mg, 82.62 μmol) and K₂CO₃ (2 M, 619.66 μL) in dioxane (3 mL) was degassed and purged with N₂ 3 times, and then the mixture was stirred at 100° C. for 16 h under an N₂ atmosphere. The reaction mixture was diluted with H₂O 10 mL and extracted with EtOAc 50 mL. The combined organic layers was dried over anhydrous Na₂SO₄, filtered and concentrated under reduced pressure to give a residue. The residue was purified by column chromatography (SiO₂, Petroleum ether/Ethyl acetate=1/1 to 0:1) to give N-[4-methyl-3-(trifluoromethyl)phenyl]-2-(2-oxaspiro[4.5]dec-7-en-8-yl)pyridine-3-carboxamide (153 mg, crude) as a colorless gum. LC-MS: (ES) m/z 417.2 (M+H⁺).

Step d) A mixture of N-[4-methyl-3-(trifluoromethyl)phenyl]-2-(2-oxaspiro[4.5]dec-7-en-8-yl) pyridine-3-carboxamide (150 mg, 360.20 mol), HCl/dioxane (4 M, 180.10 μL) and PtO₂ (16.36 mg, 72.04 mol) in MeOH (10 mL) was degassed and purged with H₂ (15 psi) (726.09 μg, 360.20 mol) for 3 times, and then the mixture was stirred at 20° C. for 3 h under H₂ atmosphere. The reaction mixture was filtered and concentrated under reduced pressure to give a residue. The residue was purified by prep-HPLC (neutral condition, column: Waters Xbridge Prep OBD C18 150*30 10μ; mobile phase: [water (10 mM NH₄HCO₃)-ACN]; B %: 35%-65%, 11 min) to give N-[4-methyl-3-(trifluoromethyl)phenyl]-2-(2-oxaspiro[4.5]decan-8-yl) piperidine-3-carboxamide (70 mg, 148.41 mol, 41.20% yield, 90% purity) as a white solid. ¹H NMR (400 MHz, CDCl₃) δ 0.88-1.17 (m, 2H), 1.22-1.43 (m, 3H), 1.46-1.65 (m, 4H), 1.76-1.91 (m, 5H), 2.17 (br d, J=13.55 Hz, 1H), 2.44 (s, 4H), 2.67-2.77 (m, 2H), 3.29 (br d, J=11.29 Hz, 1H), 3.39-3.46 (m, 1H), 3.55 (q, J=8.53 Hz, 1H), 3.83 (dt, J=18.07, 7.28 Hz, 2H), 7.23 (d, J=8.28 Hz, 1H), 7.70 (s, 1H), 7.77 (br d, J=8.28 Hz, 1H), 11.21 (br s, 1H). LC-MS: (ES) m/z 425.3 (M+H⁺).

Step e) To a solution of N-[4-methyl-3-(trifluoromethyl)phenyl]-2-(2-oxaspiro[4.5]de can-8-yl) piperidine-3-carboxamide (50 mg, 117.79 mol) in DCM (6 mL) was added DIEA (53.28 mg, 412.25 mol, 71.81 μL) and then the 2-fluoro-6-methyl-benzoyl chloride (60.98 mg, 353.36 mol) in DCM (1 mL) was added by dropwise at 0° C. The mixture was stirred at 0° C. for 2 hr. The reaction mixture was quenched by addition H₂O 5 mL, and then extracted with DCM 40 mL (20 mL*2). The combined organic layers were dried over anhydrous Na₂SO₄, filtered and concentrated under reduced pressure to give a residue. The residue combined with previous batch (15 mg) was purified by prep-HPLC (HCl condition, column: Agela ASB 150*25 mm*5 μm; mobile phase: [water (0.05% HCl)-ACN]; B %: 60%-90%, 8 min). The compound 1-(2-fluoro-6-methyl-benzoyl)-N-[4-methyl-3-(trifluoromethyl)phenyl]-2-(2-oxaspiro[4.5]decan-8-yl)piperidine-3-carboxamide (50 mg, 96% purity) was obtained as a white solid. ¹H NMR (400 MHz, METHANOL-d₄) δ 1.04-1.59 (m, 5H), 1.64-1.82 (m, 6H), 1.88 (br d, J=13.30 Hz, 1H), 2.00-2.17 (m, 2H), 2.29-2.41 (m, 3H), 2.44 (s, 3H), 2.75-2.86 (m, 1H), 3.03-3.18 (m, 1H), 3.38-3.45 (m, 1H), 3.49-3.59 (m, 1H), 3.73-3.85 (m, 2H), 4.98-5.08 (m, 1H), 7.04 (q, J=9.03 Hz, 1H), 7.11-7.22 (m, 1H), 7.31-7.41 (m, 2H), 7.63-7.73 (m, 1H), 7.96 (s, 1H), 10.31 (br d, J=9.54 Hz, 1H). LC-MS: (ES) m/z 561.3 (M+H⁺).

### Example S56: Synthesis of 2-(4-(cyclopentylamino)phenyl)-1-(2-fluoro-6-methylbenzoyl)-5-hydroxy-N-(4-methyl-3-(trifluoromethyl)phenyl)piperidine-3-carboxamide (Compound No. 42)

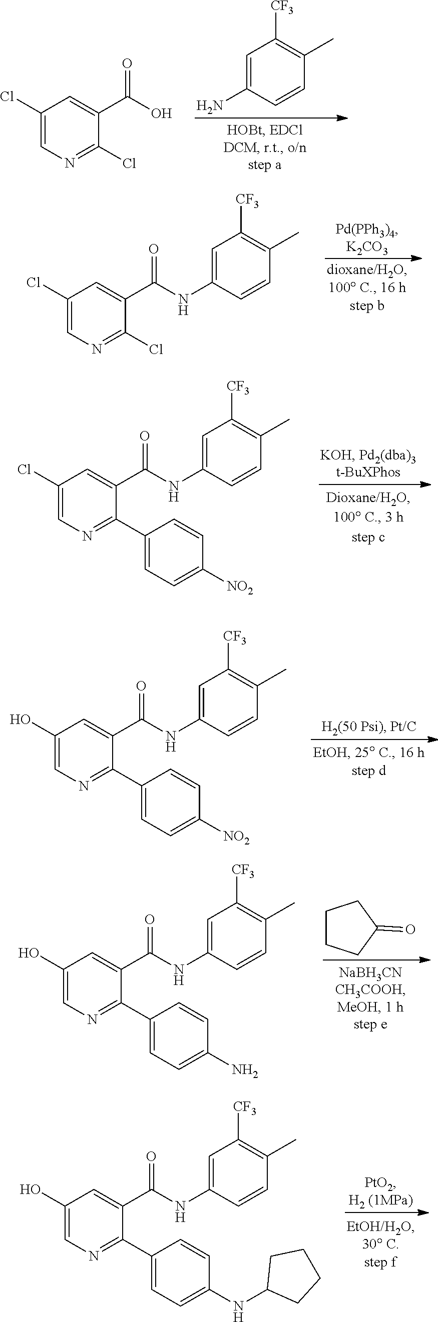

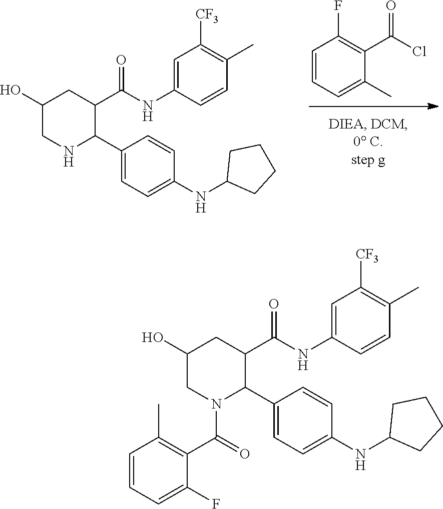 [SMILES: Cc1ccc(NC(=O)C2CC(O)CNC2c2ccc(NC3CCCC3)cc2)cc1C(F)(F)F ; Cc1cccc(F)c1C(=O)Cl ; Cc1ccc(NC(=O)C2CC(O)CN(C(=O)c3c(C)cccc3F)C2c2ccc(NC3CCCC3)cc2)cc1C(F)(F)F]

Step a) To a mixture of 2,5-dichloropyridine-3-carboxylic acid (5 g, 26.04 mmol, 31.27 μL), 4-methyl-3-(trifluoromethyl)aniline (4.33 g, 24.74 mmol, 3.55 mL) in DCM (75 mL) was added successively with EDCI (5.99 g, 31.25 mmol) and HOBt (1.06 g, 7.81 mmol) at 0° C. Then the mixture was stirred at 15° C. for 12 h. The mixture was concentrated in vacuo to give the residue. The residue was dissolved with EtOAc (100 mL) and washed with saturated NaHCO₃ solution (2×10 mL), then acidified to pH=4-5 by addition of HCl (4 M) twice, dried, filtered and concentrated in vacuo to give the desired product 2,5-dichloro-N-[4-methyl-3-(trifluoromethyl)phenyl]pyridine-3-carboxamide (7.4 g, 21.20 mmol, 81.39% yield, 100% purity) as light yellow solid. ¹H NMR (400 MHz, CDCl₃) δ 2.49 (d, J=1.22 Hz, 3 H), 7.32 (d, J=8.31 Hz, 1H), 7.75 (dd, J=8.19, 1.83 Hz, 1H), 7.82 (d, J=1.71 Hz, 1H), 8.15 (d, J=2.69 Hz, 1H), 8.39 (br s, 1H), 8.45 (d, J=2.69 Hz, 1H). LC-MS: (ES) m/z 349.0 (M+H⁺).

Step b) To a mixture of 2,5-dichloro-N-[4-methyl-3-(trifluoromethyl)phenyl]pyridine-3-carboxamide (1 g, 2.86 mmol, 31.27 μL), (4-nitrophenyl)boronic acid (573.74 mg, 3.44 mmol, 3.55 mL) in dioxane (16 mL) was added successively with Pd(PPh₃)₄ (330.98 mg, 286.42 mol) and K₂CO₃ (2 M, 4.30 mL) at 15° C. Then the mixture was stirred at 100° C. for 12 h. The mixture was concentrated in vacuo to give the residue. The residue was dissolved with EtOAc (100 mL) and washed with H₂O (2×5 mL), dried, filtered and concentrated in vacuo to give the crude product. The crude was purified by prep-HPLC (column: Boston Prime C18 150×30 mm×5 μm; mobile phase: [water (0.04% NH₃H₂O+10 mM NH₄HCO₃)-ACN]; B %: 55%-85%, 8 min) to give 5-chloro-N-[4-methyl-3-(trifluoromethyl)phenyl]-2-(4-nitrophenyl)pyridine-3-carboxamide (0.12 g, 269.86 mol, 29.40% yield, 98% purity) as light yellow solid. ¹H NMR (400 MHz, CDCl₃) δ 2.45 (s, 3H), 7.19 (s, 1H), 7.25 (d, J=8.53 Hz, 1H), 7.44 (br d, J=8.28 Hz, 1H), 7.54 (d, J=1.76 Hz, 1H), 7.92 (d, J=8.78 Hz, 2H), 8.07 (d, J=2.51 Hz, 1H), 8.31 (d, J=8.78 Hz, 2H), 8.80 (d, J=2.26 Hz, 1H). LC-MS: (ES) m/z 436.1 (M+H⁺).

Step c) A mixture of 5-chloro-N-[4-methyl-3-(trifluoromethyl)phenyl]-2-(4-nitrophenyl) pyridine-3-carboxamide (1.8 g, 4.13 mmol), Pd₂(dba)₃ (189.12 mg, 206.52 mol), t-Bu Xphos (175.40 mg, 413.05 mol) and KOH (695.29 mg, 12.39 mmol) in dioxane (40 mL)/H₂O (20 mL) was stirred at 100° C. for 16 h. The mixture was diluted with EtOAc (50 mL) and acidified to pH=4-5 by addition of HCl (2 N). The mixture was extracted with EtOAc (2×20 mL). The combined organic layers were washed with brine (2×20 mL), dried, filtered and concentrated in vacuo to give the residue. The residue was triturated with mixed solvents (22 mL, Petroleum ether/EtOAc=10/1) twice. The filter cake was dissolved with EtOAc (100 mL) and filtered through a pad of silica gel. The filtrate was concentrated in vacuo to give the pure product 5-hydroxy-N-[4-methyl-3-(trifluoromethyl)phenyl]-2-(4-nitrophenyl)pyridine-3-carboxamide (1.7 g, 3.95 mmol, 95.66% yield, 97% purity) as light brown solid. ¹H NMR (400 MHz, DMSO-d₆) δ 2.38 (s, 3H), 7.38 (d, J=8.31 Hz, 1H), 7.42 (d, J=2.69 Hz, 1H), 7.66 (br d, J=8.31 Hz, 1H), 7.82 (d, J=8.80 Hz, 2H), 7.95 (d, J=1.22 Hz, 1H), 8.24 (d, J=8.80 Hz, 2H), 8.40 (d, J=2.69 Hz, 1H), 10.73 (s, 1H), 10.76 (s, 1H). LC-MS: (ES) m/z 418.1 (M+H⁺).

Step d) To a solution of 5-hydroxy-N-[4-methyl-3-(trifluoromethyl)phenyl]-2-(4-nitrophenyl) pyridine-3-carboxamide (0.5 g, 1.20 mmol) in EtOH (15 mL) was added Pd/C (0.1 g, 10% purity) under N₂. The suspension was degassed under vacuum and purged with H₂ several times. The mixture was stirred under H₂ (15 psi) at 15° C. for 16 h. The mixture was diluted with MeOH (20 mL) and filtered through a pad of Celite. The filtrate was concentrated in vacuo to give the desired compound 2-(4-aminophenyl)-5-hydroxy-N-[4-methyl-3-(trifluoromethyl)phenyl] pyridine-3-carboxamide (0.41 g, 1.01 mmol, 83.93% yield, 95% purity) as orange solid. ¹H NMR (400 MHz, DMSO-d₆) δ 2.38 (br s, 3H), 5.22 (br s, 2H), 6.49 (d, J=8.56 Hz, 2H), 7.21 (d, J=2.69 Hz, 1H), 7.30 (d, J=8.56 Hz, 2H), 7.37 (br d, J=8.31 Hz, 1H), 7.65 (br d, J=8.07 Hz, 1H), 7.99 (s, 1H), 8.25 (d, J=2.69 Hz, 1H), 10.14 (s, 1H), 10.49 (s, 1H). LC-MS: (ES) m/z 388.1 (M+H⁺).

Step e) To a mixture of 2-(4-aminophenyl)-5-hydroxy-N-[4-methyl-3-(trifluoromethyl)-phenyl]pyridine-3-carboxamide (410.00 mg, 1.06 mmol) in MeOH (10 mL) was added cyclo-pentanone (89.03 mg, 1.06 mmol, 93.72 μL), HOAc (95.34 mg, 1.59 mmol, 90.80 μL) and NaBH₃CN (266.05 mg, 4.23 mmol) in one portion at 0° C. under N₂. The mixture was stirred at 30° C. for 16 h. The mixture was diluted with EtOAc (25 mL) and alkalified to pH=8-9 and extracted with EtOAc (3×20 mL). The combined organic layers were washed with brine, dried, filtered and concentrated in vacuo to give the crude product. The crude product was purified by silica gel column chromatography (eluted with DCM/MeOH=100/1 to 10/1) to give 2-[4-(cyclopentylamino)-phenyl]-5-hydroxy-N-[4-methyl-3-(trifluoromethyl)phenyl]pyridine-3-carboxamide (450 mg, 948.47 mol, 89.61% yield, 96% purity) as orange solid. ¹H NMR (400 MHz, DMSO-d₆) δ1.39 (dt, J=12.17, 6.02 Hz, 2H), 1.45-1.56 (m, 2H), 1.58-1.70 (m, 2H), 1.87 (dq, J=12.23, 6.03 Hz, 2H), 2.38 (s, 3H), 3.65 (dq, J=12.17, 5.97 Hz, 1H), 5.72 (d, J=6.60 Hz, 1H), 6.49 (d, J=8.80 Hz, 2H), 7.21 (d, J=2.69 Hz, 1H), 7.31-7.41 (m, 3H), 7.67 (br d, J=8.31 Hz, 1H), 7.97 (d, J=1.71 Hz, 1H), 8.25 (d, J=2.69 Hz, 1H), 10.14 (s, 1H), 10.51 (s, 1H). LC-MS: (ES) m/z 456.2 (M+H⁺).

Step f) To a solution of 2-[4-(cyclopentylamino)phenyl]-5-hydroxy-N-[4-methyl-3-(trifluoromethyl)phenyl]pyridine-3-carboxamide (0.45 g, 987.99 mol) in EtOH (10 mL)/H₂O (5 mL) was added PtO₂ (112.17 mg, 493.99 mol) under N₂. The suspension was degassed under vacuum and purged with H₂ several times. The mixture was stirred under H₂ (1 MPa) at 30° C. for 32 h. The mixture was diluted with MeOH (20 mL) and filtered through a pad of Celite. The filtrate was concentrated in vacuo to give the crude. The crude was purified by prep-HPLC (column: Venusil ASB Phenyl 150×30 mm×5 μm; mobile phase: [water (0.05% HCl)-ACN]; B %: 40%-70%, 10 min) to give 2-[4-(cyclopentylamino)phenyl]-5-hydroxy-N-[4-methyl-3-(trifluoro methyl)-phenyl]piperidine-3-carboxamide (120 mg, 260.01 mol, 30.00% yield) as off-white solid. ¹H NMR (400 MHz, DMSO-d₆) δ 1.31 (td, J=11.80, 6.24 Hz, 3H), 1.49 (br s, 2H), 1.60 (br s, 1H), 1.81 (dt, J=12.29, 5.96 Hz, 2H), 1.93-2.09 (m, 2H), 2.34 (br s, 3H), 2.79 (br d, J=14.18 Hz, 2H), 2.99 (br d, J=12.47 Hz, 1 H), 3.53-3.61 (m, 1H), 3.64 (br s, 1H), 3.85 (br d, J=3.18 Hz, 1H), 5.32 (br d, J=6.60 Hz, 1H), 5.44 (br d, J=6.36 Hz, 1H), 6.40 (br d, J=8.31 Hz, 2H), 7.02 (br d, J=8.31 Hz, 2H), 7.28 (br d, J=8.56 Hz, 1H), 7.44 (br d, J=8.07 Hz, 1H), 7.70 (s, 1H), 10.33 (s, 1H). LC-MS: (ES) m/z 462.3 (M+H⁺).

Step g) To a solution of 2-[4-(cyclopentylamino)phenyl]-5-hydroxy-N-[4-methyl-3-(tri-fluoromethyl)phenyl]piperidine-3-carboxamide (10 mg, 19.93 mol) and DIEA (5.15 mg, 39.87 mol, 6.94 μL) in DCM (0.5 mL) was added dropwise of a solution of 2-fluoro-6-methyl-benzoyl chloride (3.27 mg, 18.94 mol) in DCM (0.2 mL) at 0° C. The mixture was stirred at 0° C. for 10 min. The mixture was diluted with DCM (120 mL), washed with H₂O (2×10 mL), dried, filtered and concentrated in vacuo to give the crude product. The crude product was purified by prep-HPLC (column: Venusil ASB Phenyl 150×30 mm×5 μm; mobile phase: [water (0.05% HCl)-ACN]; B %: 45%-75%, 9 min) to give 2-[4-(cyclopentylamino)phenyl]-1-(2-fluoro-6-methyl-benzoyl)-5-hydroxy-N-[4-methyl-3-(trifluoromethyl)phenyl]piperidine-3-carboxamide (5 mg, 8.37 mol, 41.97% yield, 100% purity) as white solid. ¹H NMR (400 MHz, METHANOL-d₄) δ1.67 (br s, 4H), 1.82 (br d, J=4.02 Hz, 2H), 1.99 (br d, J=4.27 Hz, 2H), 2.06 (s, 2H), 2.12-2.26 (m, 1H), 2.28-2.36 (m, 1H), 2.36-2.48 (m, 5H), 2.97 (dd, J=12.80, 11.04 Hz, 1H), 3.24-3.30 (m, 1H), 3.41-3.54 (m, 1H), 3.71-3.87 (m, 1H), 3.88-3.99 (m, 1H), 6.44-6.52 (m, 1H), 6.97-7.14 (m, 2H), 7.19 (d, J=7.78 Hz, 1H), 7.25-7.43 (m, 4H), 7.46-7.58 (m, 1H), 7.72 (dd, J=12.17, 8.66 Hz, 2H), 7.78-7.88 (m, 1H), 10.26 (d, J=10.79 Hz, 1H). LC-MS: (ES) m/z 598.3 (M+H⁺).

### Example S57: Synthesis of cis-4-(4-(cyclopentylamino)phenyl)-N-(4-methyl-3-(trifluoromethyl)phenyl)-6-oxo-2,3,4,6,11,11a-hexahydro-1H-pyrido[1,2-b]isoquinoline-3-carboxamide (Compound No. 43)

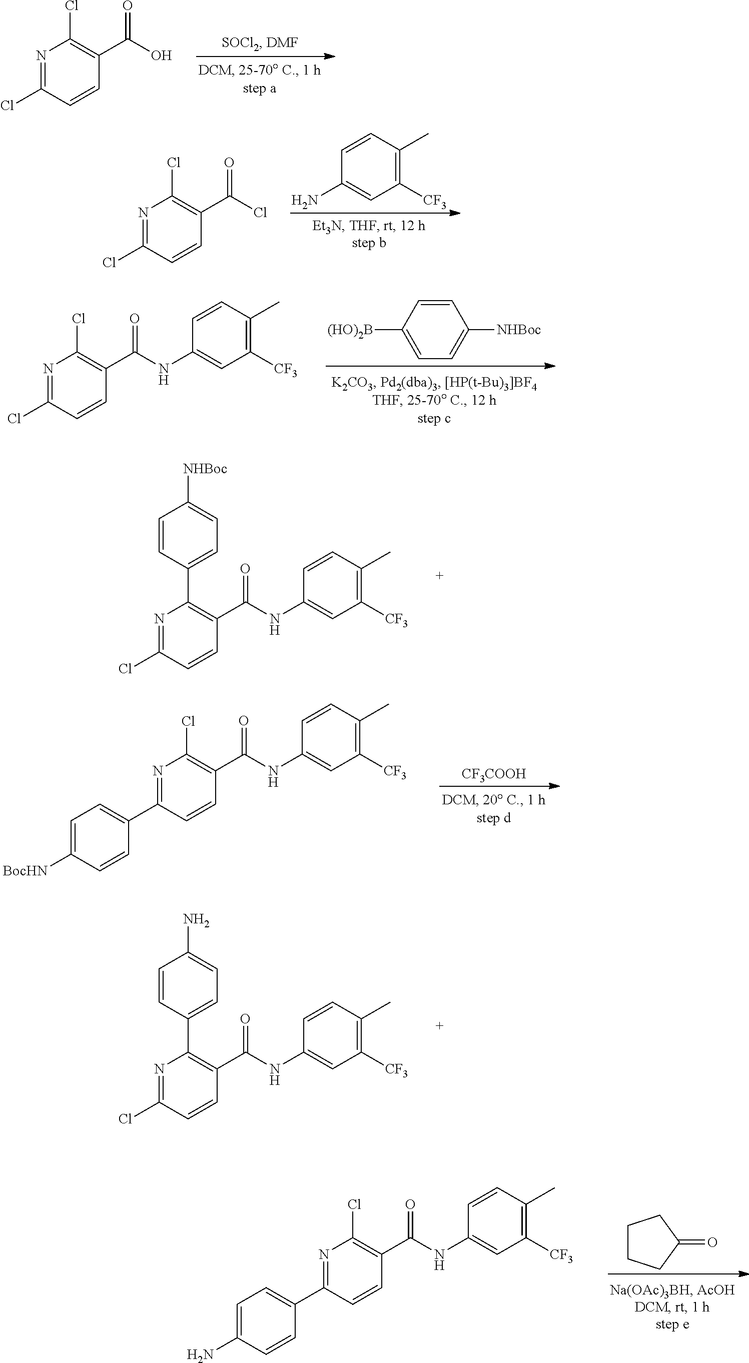

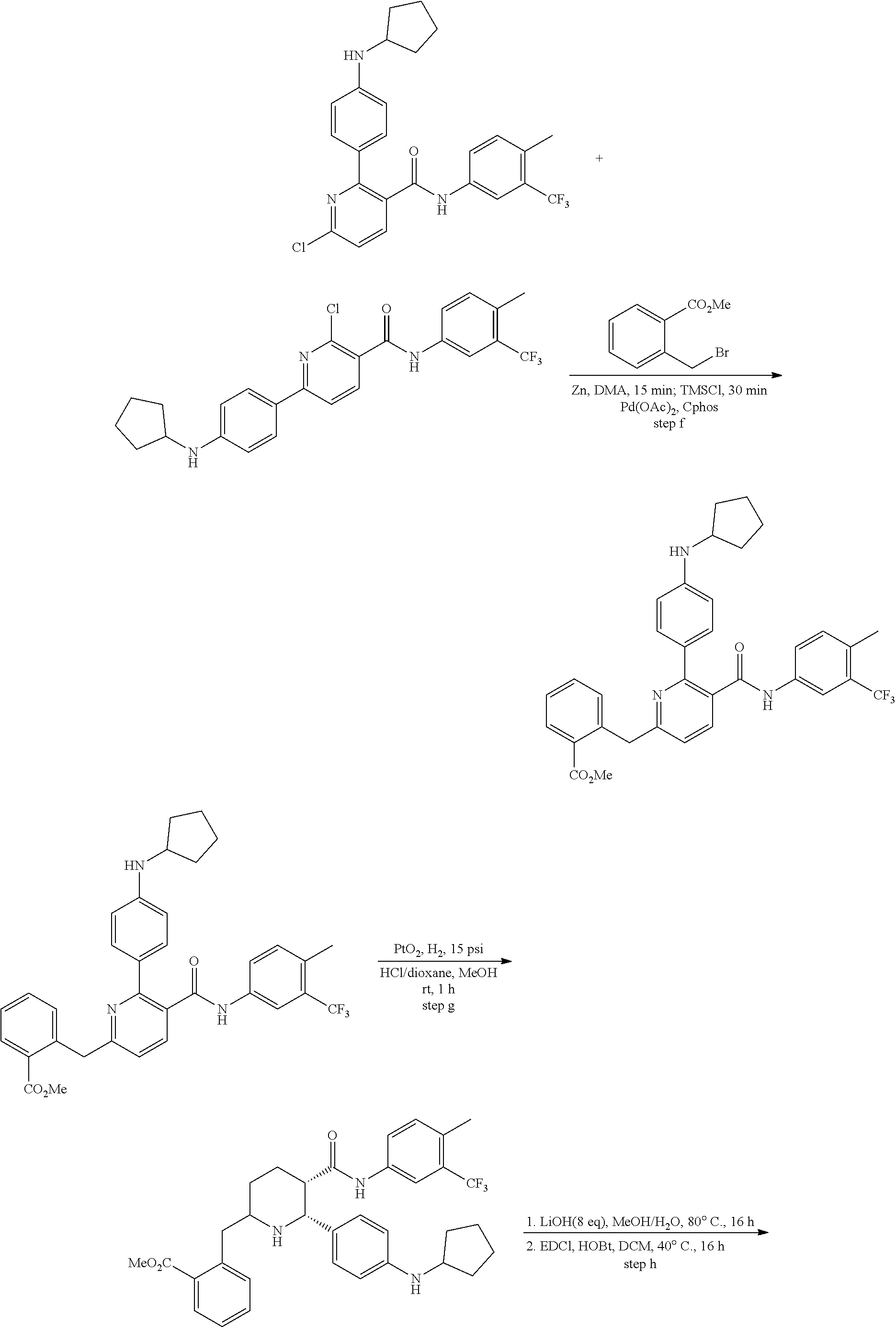 [SMILES: Cc1ccc(NC(=O)c2ccc(Cl)nc2-c2ccc(NC3CCCC3)cc2)cc1C(F)(F)F ; Cc1ccc(NC(=O)c2ccc(-c3ccc(NC4CCCC4)cc3)nc2Cl)cc1C(F)(F)F ; COC(=O)c1ccccc1CBr ; COC(=O)c1ccccc1Cc1ccc(C(=O)Nc2ccc(C)c(C(F)(F)F)c2)c(-c2ccc(NC3CCCC3)cc2)n1 ; COC(=O)c1ccccc1Cc1ccc(C(=O)Nc2ccc(C)c(C(F)(F)F)c2)c(-c2ccc(NC3CCCC3)cc2)n1 ; COC(=O)c1ccccc1CC1CC[C@H](C(=O)Nc2ccc(C)c(C(F)(F)F)c2)[C@H](c2ccc(NC3CCCC3)cc2)N1]

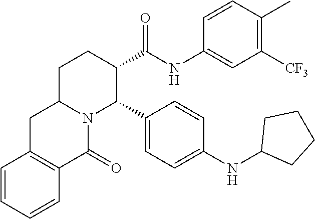 [SMILES: Cc1ccc(NC(=O)[C@H]2CCC3Cc4ccccc4C(=O)N3[C@H]2c2ccc(NC3CCCC3)cc2)cc1C(F)(F)F]

Step a) To a mixture of 2,6-dichloropyridine-3-carboxylic acid (10 g, 52.08 mmol) and DMF (380.70 mg, 5.21 mmol, 400.73 μL) in DCM (20 mL) was added thionyl chloride (30.98 g, 260.42 mmol, 18.89 mL) in one portion at 25° C. The mixture was stirred at 70° C. for 1 h. The reaction mixture was concentrated under reduced pressure to give a crude 2,6-dichloropyridine-3-carbonyl chloride (10.8 g, crude) as a light yellow solid. The crude product was used for the next step without further purification. LC-MS: (ES) m/z 206.1 (M+H⁺).

Step b) To a mixture of 2,6-dichloropyridine-3-carbonyl chloride (10.8 g, 51.32 mmol) and 4-methyl-3-(trifluoromethyl)aniline (8.99 g, 51.32 mmol, 7.37 mL) in THF (30 mL) was added Et₃N (7.79 g, 76.98 mmol, 10.71 mL) in one portion at 0° C. The mixture was stirred at 25° C. for 12 h. The reaction mixture was filtered and concentrated under reduced pressure to give a residue. The residue was purified by column chromatography (SiO₂, Petroleum ether/Ethyl acetate=100/0 to 10:1) to give a crude product. The crude product was triturated with EtOAc (10 ml) and petroleum ether (50 ml) at 25° C. for 3 h to give the target product 2,6-dichloro-N-[4-methyl-3-(trifluoromethyl)-phenyl]pyridine-3-carboxamide (12.7 g, 36.38 mmol, 70.88% yield) as a white solid. ¹H NMR (400 MHz, CDCl₃) δ 2.50 (d, J=1.22 Hz, 3H), 7.33 (d, J=8.31 Hz, 1H), 7.77 (dd, J=8.07, 1.96 Hz, 1H), 7.83 (d, J=1.71 Hz, 1H), 8.21 (d, J=2.45 Hz, 1H), 8.26 (br s, 1H), 8.49 (d, J=2.69 Hz, 1H). LC-MS: (ES) m/z 349.0 (M+H⁺).

Step c) To a mixture of [4-(tert-butoxycarbonylamino)phenyl]boronic acid (4.58 g, 19.33 mmol) and 2,6-dichloro-N-[4-methyl-3-(trifluoromethyl)phenyl]pyridine-3-carboxamide (10 g, 23.20 mmol) in THF (50 mL) and H₂O (5 mL) added Pd₂(dba)₃ (885.20 mg, 966.68 mol), tritert-butylphosphonium; tetrafluoroborate (560.92 mg, 1.93 mmol) and KF (3.37 g, 58.00 mmol, 1.36 mL) in one portion at 25° C. under N₂. The mixture was stirred at 70° C. for 12 hours. The reaction mixture was concentrated under reduced pressure to remove solvent. The residue was diluted with brine 50 mL and extracted with EtOAc 150 mL (50 mL×3). The combined organic layers were dried over Na₂SO₄, filtered and concentrated under reduced pressure to give a residue. The residue was purified by column chromatography (SiO₂, petroleum ether/ethyl acetate=100/1 to 5:1) to give the target product tert-butyl N-[4-[6-chloro-3-[[4-methyl-3-(trifluoromethyl)phenyl]carbamoyl]-2-pyridyl]phenyl]carbamate and tert-butylN-[4-[6-chloro-5-[[4-methyl-3-(trifluoromethyl)phenyl]-carbamoyl]-2-pyridyl]phenyl]carbamate (mixture, 7.5 g) as a light yellow solid. LC-MS: (ES) m/z 506.1 (M+H⁺).

Step d) To a mixture of tert-butyl N-[4-[6-chloro-5-[[4-methyl-3-(trifluoromethyl)-phenyl]carbamoyl]-2-pyridyl]phenyl]carbamate (14.82 mmol) and tert-butyl N-[4-[6-chloro-3-[[4-methyl-3-(trifluoromethyl)phenyl]carbamoyl]-2-pyridyl]phenyl]carbamate in CH₂Cl₂ (3 mL) was added CF₃COOH (9.24 g, 81.04 mmol, 6 mL) in one portion at 0° C. The mixture was stirred at 25° C. for 1 hour. The reaction mixture was concentrated under reduced pressure to give a crude 2-(4-aminophenyl)-6-chloro-N-[4-methyl-3-(trifluoromethyl)phenyl]pyridine-3-carboxamide and 6-(4-aminophenyl)-2-chloro-N-[4-methyl-3-(trifluoromethyl)phenyl]pyridine-3-carboxamide (crude mixture 7.45 g) as a light yellow oil. The crude was used for the next step without further purification. LC-MS: (ES) m/z 406.1 (M+H⁺).

Step e) To a mixture of cyclopentanone (3.11 g, 36.96 mmol, 3.27 mL) and 2-(4-aminophenyl)-6-chloro-N-[4-methyl-3-(trifluoromethyl)phenyl]pyridine-3-carboxamide and 6-(4-aminophenyl)-2-chloro-N-[4-methyl-3-(trifluoromethyl)phenyl]pyridine-3-carboxamide (7.45 g, mixture) in DCM (50 mL) was added AcOH (1.66 g, 27.72 mmol, 1.59 mL) and NaBH(OAc)₃ (3.92 g, 18.48 mmol) in one portion at 0° C. under N₂. The mixture was stirred at 30° C. for 1 hour. The reaction mixture was filtered and concentrated under reduced pressure to give a residue. The crude product was purified by prep-HPLC: column: SANPONT C18, 250×50 mm×10 m, 100 A; mobile phase: [water (10 mM NH₄HCO₃)-ACN]; B %: 60%-90%, 25 min to give the target product 6-chloro-2-[4-(cyclopentylamino)phenyl]-N-[4-methyl-3-(trifluoromethyl)phenyl]pyridine-3-carboxamide (3.51 g, 7.41 mmol, 40.07% yield) as a light yellow solid. ¹H NMR (400 MHz, CDCl₃) δ 1.38-1.52 (m, 2H), 1.60-1.67 (m, 2H), 1.68-1.78 (m, 2H), 2.01 (dq, J=12.7, 6.4 Hz, 2H), 2.40 (d, J=1.0 Hz, 3H), 3.80 (quin, J=6.2 Hz, 1H), 3.97 (br s, 1H), 6.60 (d, J=8.6 Hz, 2H), 7.17 (d, J=8.3 Hz, 1H), 7.22 (s, 1H), 7.25 (s, 1H), 7.26-7.33 (m, 2H), 7.39 (br d, J=8.3 Hz, 1H), 7.50 (d, J=8.6 Hz, 2H), 8.07 (d, J=8.3 Hz, 1H). LC-MS: (ES) m/z 474.1 (M+H⁺).

Step f) To a solution of zinc (363 mg, 5.55 mmol) in DMA (20 mL) was added 1,2-dibromoethane (63.42 mg, 337.61 mol, 25.47 μL) by dropwise, then the mixture was stirred at 65° C. for 30 min. Later it was cooled to 25° C. The chloro(trimethyl)silane (27.51 mg, 253.21 mol, 32.14 μL) was added at 25° C. dropwise. The mixture was stirred at 25° C. for 30 min. Then the methyl 2-(bromomethyl)benzoate (1.0 g, 4.37 mmol) in DMA (5 mL) was added to the mixture dropwise. The reaction mixture was stirred at 25° C. for 1.5 h. The 6-chloro-2-[4-(cyclopentylamino)phenyl]-N-[4-methyl-3-(trifluoromethyl)phenyl]pyridine-3-carboxamide (1 g, 2.11 mmol), Pd(OAc)₂ (47.37 mg, 211.01 μmol) and 2-(2-dicyclo hexylphosphanylphenyl)-N1,N1,N3,N3-tetramethyl-benzene-1,3-diamine (92.13 mg, 211.01 mol) in DMA (6 mL) was added to the mixture by dropwise. Then the mixture was stirred at 25° C. for 16 h under N2 atmosphere. The reaction mixture was quenched by addition aq. NH₄Cl 50 mL, and then extracted with EtOAc 300 mL (150 mL×2). The combined organic layers were washed with brine (30 mL), dried over anhydrous Na₂SO₄, filtered and concentrated under reduced pressure to give a residue. The residue was purified by flash silica gel chromatography (ISCO®; 20 g SepaFlash® Silica Flash Column, eluent of 0˜25% ethyl acetate/petroleum ether gradient@ 30 mL/min) to give methyl 2-[[6-[4-(cyclopentylamino)phenyl]-5-[[4-methyl-3-(trifluoromethyl) phenyl]carbamoyl]-2-pyridyl]methyl]benzoate (1.27 g, 2.10 mmol, 99.35% yield, 97% purity) as a yellow solid. ¹H NMR (400 MHz, CDCl₃) δ 1.48 (dt, J=12.17, 6.02 Hz, 2H), 1.62-1.68 (m, 2H), 1.72-1.79 (m, 2H), 1.99-2.06 (m, 2H), 2.42 (s, 3H), 3.79-3.84 (m, 1H), 3.85 (s, 3H), 4.66 (s, 2H), 6.64 (d, J=8.56 Hz, 2H), 7.05 (d, J=8.07 Hz, 1H), 7.17 (br d, J=11.25 Hz, 2H), 7.30-7.44 (m, 4H), 7.47-7.53 (m, 3H), 7.97 (dd, J=7.95, 1.10 Hz, 1H), 8.04 (d, J=7.83 Hz, 1H). LC-MS: (ES) m/z 588.24 (M+H⁺).

Step g) A mixture of methyl 2-[[6-[4-(cyclopentylamino)phenyl]-5-[[4-methyl-3-(tri-fluoromethyl)phenyl]carbamoyl]-2-pyridyl]methyl]benzoate (500 mg, 850.88 mol), PtO₂ (101 mg, 444.78 mol) and HCl/dioxane (4 M, 426.00 μL) in MeOH (15 mL) was degassed and purged with H₂ (15 psi) 3 times. Then the mixture was stirred at 20° C. for 7 h under H₂ atmosphere. The reaction mixture was filtered and concentrated under reduced pressure to give a residue. The residue was purified by prep-HPLC (HCl condition, column: Agela ASB 150×25 mm×5 μm; mobile phase: [water (0.05% HCl)-ACN]; B %: 40%-70%, 8 min) to give cis-methyl 2-[[6-[4-(cyclopentylamino)-phenyl]-5-[[4-methyl-3-(trifluoromethyl)phenyl]carbamoyl]-2-piperidyl]methyl]benzoate (250 mg, 396.74 mol, 46.63% yield, 100% purity, HCl) as a white solid. ¹H NMR (400 MHz, METHANOL-d4) 1.59-1.75 (m, 4H), 1.79-1.85 (m, 2H), 1.88-1.99 (m, 2H), 2.05-2.16 (m, 1H), 2.20 (br s, 2H), 2.40 (s, 3H), 3.23 (br s, 1H), 3.39 (br dd, J=12.96, 8.31 Hz, 1H), 3.66 (dd, J=12.96, 5.62 Hz, 1H), 3.87 (br d, J=5.38 Hz, 1H), 3.92-3.96 (m, 1H), 3.97 (s, 3H), 4.81 (br s, 1 H), 7.28 (d, J=8.31 Hz, 1H), 7.40-7.47 (m, 1H), 7.47-7.54 (m, 2H), 7.55-7.63 (m, 3H), 7.77 (br d, J=8.31 Hz, 2H), 7.88 (s, 1H), 8.01 (d, J=7.83 Hz, 1H), 10.18 (s, 1H). LC-MS: (ES) m/z 594.3 (M+H⁺).

Step h) To a solution of cis-methyl 2-[[6-[4-(cyclopentylamino)phenyl]-5-[[4-methyl-3-(trifluoromethyl)phenyl]carbamoyl]-2-piperidyl]methyl]benzoate (200 mg, 317.39 mol, HCl) in MeOH (10 mL) and H₂O (3 mL) was added LiOH (60.81 mg, 2.54 mmol). The mixture was stirred at 80° C. for 16 h. The reaction was concentrated and re-dissolved in DCM (15 mL). Then EDCI (182.53 mg, 952.17 mol), HOBt (42.89 mg, 317.39 mol) and 4-METHYLMORPHOLINE (122.00 mg, 1.21 mmol, 132.60 μL) were added and the mixture was stirred at 40° C. for 16 h. The reaction mixture was concentrated under reduced pressure to give a residue. The residue was purified by flash silica gel chromatography (ISCO®; 20 g SepaFlash® Silica Flash Column, eluent of 0˜36% ethyl acetate/petroleum ether gradient@35 mL/min). Compound cis-4-[4-(cyclopentyl-amino)phenyl]-N-[4-methyl-3-(trifluoromethyl)phenyl]-6-oxo-1,2,3,4,11,11a-hexahydrobenzo[b]-quinolizine-3-carboxamide (150 mg, 240.37 mol, 75.73% yield, 90% purity) was obtained as a white solid. ¹H NMR (400 MHz, METHANOL-d₄) δ 1.39-1.51 (m, 2H), 1.54-1.64 (m, 2H), 1.67-1.84 (m, 3H), 1.88-2.11 (m, 6H), 2.44 (d, J=1.25 Hz, 3H), 2.93-3.09 (m, 2H), 3.72 (quin, J=6.27 Hz, 1H), 3.97-4.10 (m, 1H), 6.05 (d, J=4.02 Hz, 1H), 6.55 (d, J=8.53 Hz, 2 H), 7.06 (d, J=8.53 Hz, 2H), 7.25-7.39 (m, 3H), 7.45-7.53 (m, 1H), 7.62 (dd, J=8.28, 2.01 Hz, 1H), 7.91-7.98 (m, 2H). LC-MS: (ES) m/z 562.3 (M+H⁺).

### Example S58: Synthesis of (3S,4R)-4-(4-(cyclopentylamino)phenyl)-N-(4-methyl-3-(trifluoro-methyl)phenyl)-6-oxo-1,2,3,4,6,11,12,12a-octahydrobenzo[e]pyrido[1,2-a]azepine-3-carboxamide and (3R,4S)-4-(4-(cyclopentylamino)phenyl)-N-(4-methyl-3-(trifluoromethyl)phenyl)-6-oxo-1,2,3,4,6,11,12,12a-octahydrobenzo[e]pyrido[1,2-a]azepine-3-carboxamide (Compound Nos. 44 and 45)

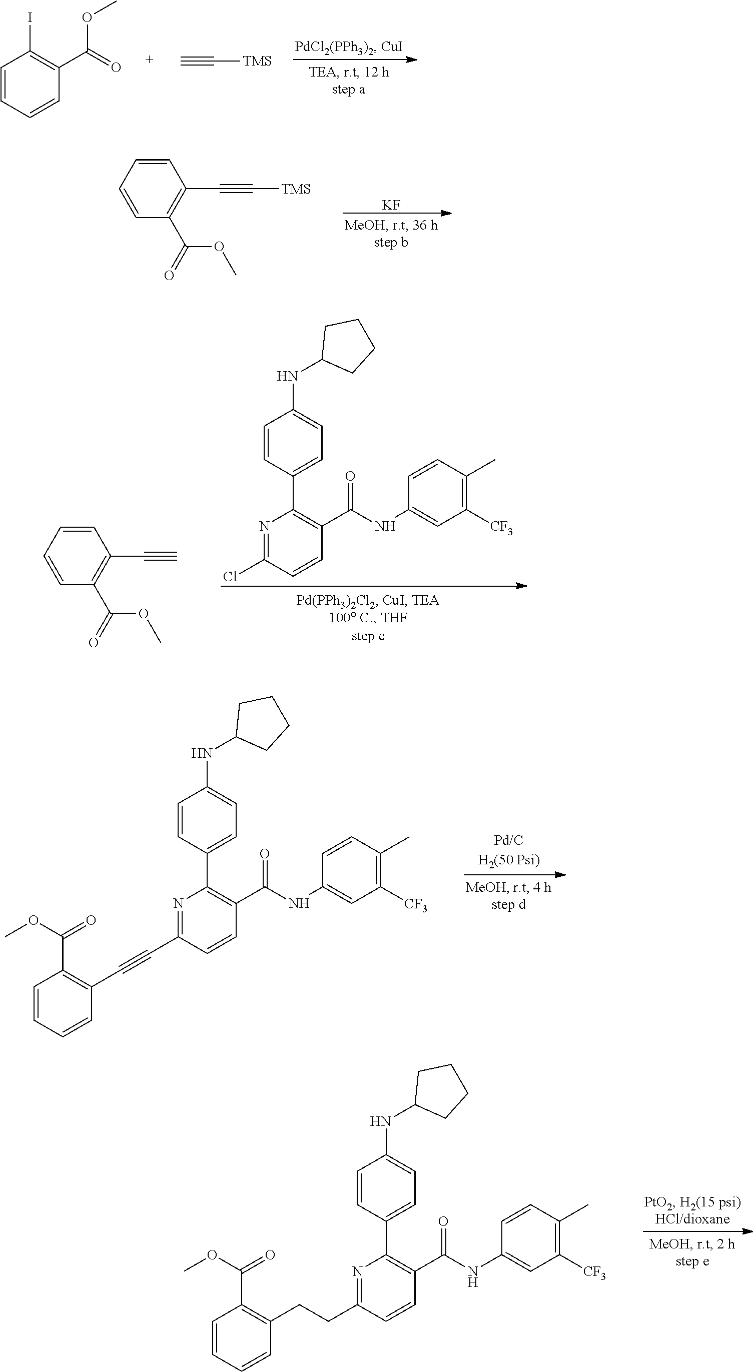

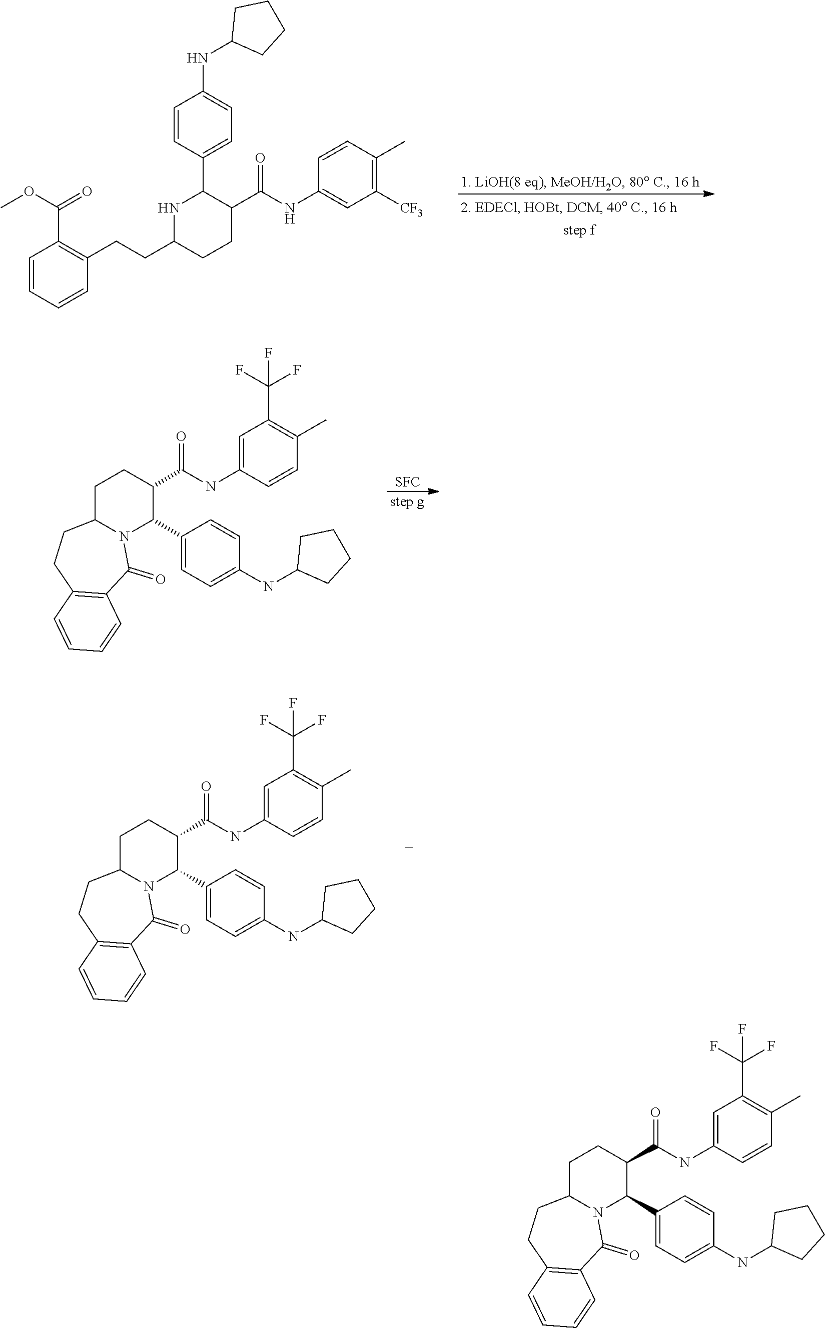

Step a) To a mixture of methyl 2-iodobenzoate (900 mg, 3.43 mmol, 505.62 μL), CuI (32.71 mg, 171.73 mol, 0.05 eq) and dichloropalladium was added triphenylphosphane (120.53 mg, 171.73 mol) in TEA (40 mL) and ethynyl(trimethyl)silane (337.33 mg, 3.43 mmol, 475.79 μL) in TEA (5 mL) at 20° C. under N₂. The mixture was filtered, washed with brine, and extracted with EtOAc (2×10 mL). The combined extracts were dried over MgSO₄ and concentrated under vacuum to yield the residue. The residue was purified by column chromatography (SiO₂, petroleum ether/ethyl acetate=100/1 to 10:1) to give the target product methyl 2-(2-trimethylsilylethynyl)-benzoate (780 mg, 3.36 mmol, 97.74% yield) as a colorless oil. ¹H NMR (400 MHz, CDCl₃) δ 0.27 (s, 9H), 3.83-3.98 (m, 4H), 7.15 (td, J=7.7, 1.8 Hz, 1H), 7.32-7.47 (m, 2H), 7.58 (d, J=7.8 Hz, 1H), 7.79 (dd, J=7.8, 1.5 Hz, 1 H), 7.90 (dd, J=7.8, 0.8 Hz, 1H), 7.99 (d, J=7.8 Hz, 1H). LC-MS: (ES) m/z 233.1 (M+H⁺).

Step b) To a mixture of methyl 2-(2-trimethylsilylethynyl)benzoate (780 mg, 3.36 mmol) in MeOH (3 mL) was added KF (390.06 mg, 6.71 mmol, 157.28 μL) in one portion at 25° C. under N₂. The mixture was stirred at 25° C. for 36 hours. The reaction mixture was concentrated under reduced pressure to remove MeOH (3 mL). The residue was extracted with EtOAc (20 mL×3). The combined organic layers were washed with 0.1 M HCl (15 mL) and brine (15 mL×3), dried over Na₂SO₄, then filtered and concentrated under reduced pressure to give a residue. The residue was purified by column chromatography (SiO₂, petroleum ether/ethyl acetate=100/0 to 20:1) to give the target product methyl 2-ethynylbenzoate (280.5 mg, 1.75 mmol, 52.17% yield) as a brown oil. ¹H NMR (400 MHz, CDCl₃) δ 3.40 (s, 1H), 3.93 (s, 3H), 7.37-7.43 (m, 1H), 7.48 (td, J=7.6, 1.2 Hz, 1H), 7.62 (d, J=7.6 Hz, 1H), 7.94 (dd, J=7.7, 0.9 Hz, 1H). LC-MS: (ES) m/z 161.05 (M+H⁺).

Step c) To a solution of 6-chloro-2-[4-(cyclopentylamino)phenyl]-N-[4-methyl-3-(trifluoromethyl)phenyl]pyridine-3-carboxamide (1.4 g, 2.95 mmol) and methyl 2-ethynylbenzoate (1.00 g, 6.24 mmol) in THF (40 mL) was added CuI (28.13 mg, 147.71 mol), PPh₃ (77.48 mg, 295.41 mol) and TEA (4.69 g, 46.34 mmol, 6.45 mL), then the mixture was stirred at 25° C. for 3 min. Pd(PPh₃)₂Cl₂ (100 mg, 142.47 mol) was added and the mixture was heated at 100° C. for 16 h under N₂ atmosphere. The reaction mixture was concentrated under reduced pressure to remove THF. The residue was diluted with H₂O (100 mL) and extracted with EtOAc (300 mL×2). The combined organic layers were dried over anhydrous Na₂SO₄, then filtered and concentrated under reduced pressure to give a residue. The residue was purified by flash silica gel chromatography (ISCO®; 24 g SepaFlash® Silica Flash Column, Eluent of 0˜30% ethyl acetate/petroleum ether gradient@ 30 mL/min). The compound methyl 2-[2-[6-[4-(cyclopentylamino)phenyl]-5-[[4-methyl-3-(trifluoromethyl)phenyl]carbamoyl]-2-pyridyl]ethynyl]benzoate (1.8 g, 2.71 mmol, 91.76% yield, 90% purity) was obtained as a brown gum. ¹H NMR (400 MHz, CDCl₃) δ 1.48 (dt, J=12.23, 6.05 Hz, 2H), 1.61-1.80 (m, 4H), 1.98-2.05 (m, 2H), 2.43 (s, 3H), 3.79-3.87 (m, 1H), 3.99 (s, 3H), 6.65 (d, J=8.53 Hz, 2H), 7.19 (br d, J=8.28 Hz, 1H), 7.33 (s, 1H), 7.40-7.48 (m, 2H), 7.52-7.57 (m, 2H), 7.60 (d, J=8.03 Hz, 1H), 7.76 (d, J=7.78 Hz, 1H), 8.03 (dd, J=7.91, 1.13 Hz, 1H), 8.21 (d, J=8.03 Hz, 1H). LC-MS: (ES) m/z 598.2 (M+H⁺).

Step d) To a solution of methyl 2-[2-[6-[4-(cyclopentylamino)phenyl]-5-[[4-methyl-3-(trifluoromethyl)phenyl]carbamoyl]-2-pyridyl]ethynyl]benzoate (1.0 g, 1.67 mmol) in MeOH (100 mL) was added Pd/C (wet) (400 mg, 10% purity). The mixture was degassed and purged with H₂ (15 psi) 3 times, and then the mixture was stirred at 20° C. for 16 h under H₂ atmosphere. The reaction mixture was filtered and concentrated under reduced pressure to give a crude product (1.1 g). To a solution of the crude product (1.1 g) in MeOH (50 mL) was added Pd/C (wet) (700 mg, 10% purity). The mixture was degassed and purged with H₂ (50 psi) 3 times, and then the mixture was stirred at 20° C. for another 4 hr under H₂ atmosphere. The reaction mixture was filtered and concentrated under reduced pressure to give a residue. The residue was purified by flash silica gel chromatography (ISCO®; 20 g SepaFlash® Silica Flash Column, eluent of 0-25% ethyl acetate/petroleum ether gradient@35 mL/min) to give methyl 2-[2-[6-[4-(cyclopentylamino) phenyl]-5-[[4-methyl-3-(trifluoromethyl) phenyl]carbamoyl]-2-pyridyl]ethyl] benzoate (785 mg, 1.25 mmol, 68.07% yield, 96% purity) as a light yellow solid. ¹H NMR (400 MHz, CDCl₃) δ 1.48 (dq, J=12.23, 5.96 Hz, 2H), 1.62-1.69 (m, 2H), 1.71-1.81 (m, 2H), 2.00-2.06 (m, 2H), 2.42 (s, 3H), 3.17-3.26 (m, 2H), 3.45 (dd, J=9.29, 6.53 Hz, 2H), 3.80-3.88 (m, 1H), 3.92 (s, 3H), 6.66 (d, J=8.53 Hz, 2H), 7.17-7.23 (m, 3H), 7.28-7.34 (m, 3H), 7.39-7.46 (m, 2 H), 7.51 (d, J=8.53 Hz, 2H), 7.93 (d, J=7.78 Hz, 1H), 8.09 (d, J=8.03 Hz, 1H). LC-MS: (ES) m/z 602.3 (M+H⁺).

Step e) A mixture of methyl 2-[2-[6-[4-(cyclopentylamino)phenyl]-5-[[4-methyl-3-(trifluoromethyl)phenyl]carbamoyl]-2-pyridyl]ethyl]benzoate (400 mg, 664.83 mol), PtO₂ (80.00 mg, 352.36 mol) and HCl/dioxane (4 M, 334.00 μL) in MeOH (10 mL) was degassed and purged with H₂ (15 psi) 3 times, and then the mixture was stirred at 20° C. for 4 h under an H₂ atmosphere. The reaction mixture was filtered and concentrated under reduced pressure to give a residue. The residue was purified by prep-HPLC (HCl condition, column: Agela ASB 150×25 mm×5 μm; mobile phase: [water (0.05% HCl)-ACN]; B %: 45%-75%, 8 min). The compound cis-methyl2-[2-[6-[4-(cyclopentylamino)phenyl]-5-[[4-methyl-3-(trifluoromethyl)phenyl]carbamoyl]-2-piperidyl]ethyl]benzoate (HCl) (270 mg. 95% purity) was obtained as a light yellow solid. ¹H NMR (400 MHz, METHANOL-d₄) δ 1.64 (br d, J=3.18 Hz, 4H), 1.80 (br s, 2H), 1.87-1.98 (m, 2H), 2.05-2.26 (m, 4H), 2.26-2.35 (m, 2 H), 2.40 (s, 3H), 2.97-3.09 (m, 1H), 3.21-3.29 (m, 2H), 3.48-3.59 (m, 1H), 3.90-3.96 (m, 4H), 4.80 (br s, 1H), 7.27 (d, J=8.31 Hz, 1H), 7.32-7.39 (m, 1H), 7.42 (d, J=6.85 Hz, 1H), 7.45-7.57 (m, 4H), 7.77 (br d, J=8.31 Hz, 2H), 7.86 (s, 1H), 7.94-7.99 (m, 1H), 10.19 (br s, 1H). LC-MS: (ES) m/z 608.3 (M+H⁺).

Step f) To a solution of cis-methyl2-[2-[6-[4-(cyclopentylamino)phenyl]-5-[[4-methyl-3-(trifluoromethyl)phenyl]carbamoyl]-2-piperidyl]ethyl]benzoate (100 mg, 164.55 mol) in MeOH (1 mL) and H₂O (0.3 mL) was added LiOH (31.53 mg, 1.32 mmol). The mixture was stirred at 80° C. for 16 h. The reaction was concentrated and re-dissolved in DCM (1.5 mL). Then EDCI (94.64 mg, 493.66 mol), HOBt (22.24 mg, 164.55 mol) and 4-methylmorpholine (63.25 mg, 625.30 mol, 68.75 μL) were added and the mixture was stirred at 40° C. for 16 h. The reaction mixture was concentrated under reduced pressure to give a residue. The residue was purified by flash silica gel chromatography (ISCO®; 12 g SepaFlash® Silica Flash Column, Eluent of 0˜30% ethyl acetate/petroleum ether gradient@35 mL/min) to give cis-10-[4-(cyclopentylamino)phenyl]-N-[4-methyl-3-(trifluoromethyl) phenyl]-12-oxo-6,6a,7,8,9,10-hexahydro-5H-pyrido[1,2-b][2]benzazepine-9-carboxamide (40 mg, 64.62 mol, 39.27% yield, 93% purity) as a white solid. ¹H NMR (400 MHz, CDCl₃) δ 1.38 (br d, J=6.60 Hz, 2H), 1.52-1.61 (m, 2H), 1.67-1.76 (m, 3H), 1.88-1.99 (m, 4H), 2.02-2.18 (m, 2H), 2.33 (s, 3H), 2.43-2.53 (m, 2H), 2.60-2.73 (m, 1H), 3.18 (dt, J=11.55, 5.84 Hz, 1H), 3.58 (br s, 1H), 3.63-3.71 (m, 1H), 3.76 (br dd, J=12.84, 5.26 Hz, 1H), 6.38 (d, J=8.56 Hz, 2H), 6.76 (br d, J=6.11 Hz, 1H), 7.01 (br d, J=8.31 Hz, 1H), 7.07 (d, J=7.09 Hz, 1H), 7.22-7.26 (m, 1H), 7.28-7.35 (m, 2H), 7.39 (s, 1H), 7.50 (d, J=8.56 Hz, 2H), 7.66 (d, J=7.34 Hz, 1H), 8.80 (br s, 1H). LC-MS: (ES) m/z 576.4 (M+H⁺).

Step g) The cis-10-[4-(cyclopentylamino)phenyl]-N-[4-methyl-3-(trifluoromethyl)-phenyl]-12-oxo-6,6a,7,8,9,10-hexahydro-5H-pyrido[1,2-b][2]benzazepine-9-carboxamide (15 mg, 26.06 μmol) was separated by SFC (column: DAICEL CHIRALCEL OD-H (250 mm×30 mm, 5 m); mobile phase: [0.1%₀NH₃H₂O ETOH]; B %: 30%-30%, 8 min) to give (9S,10R)-10-[4-(cyclo pentylamino)phenyl]-N-[4-methyl-3-(trifluoromethyl)phenyl]-12-oxo-6,6a,7,8,9,10-hexahydro-5H-pyrido[1,2-b][2]benzazepine-9-carboxamide (5 mg, 8.69 mol, 33.33% yield, 100% purity) was obtained as a white solid (¹H NMR (400 MHz, CDCl₃) δ 1.34-1.41 (m, 2H), 1.53-1.60 (m, 2H), 1.69-1.78 (m, 3H), 1.88-1.98 (m, 4H), 2.02-2.14 (m, 2H), 2.32 (s, 3H), 2.44-2.52 (m, 2H), 2.63-2.75 (m, 1H), 3.20 (dt, J=11.86, 5.81 Hz, 1H), 3.39-3.60 (m, 1H), 3.65 (dt, J=12.41, 6.14 Hz, 1H), 3.72-3.82 (m, 1H), 6.35 (d, J=8.80 Hz, 2H), 6.83 (d, J=6.60 Hz, 1H), 6.97 (d, J=8.31 Hz, 1H), 7.07 (d, J=7.34 Hz, 1H), 7.23-7.26 (m, 1H), 7.28-7.35 (m, 2H), 7.36 (s, 1H), 7.52 (d, J=8.56 Hz, 2H), 7.68 (dd, J=7.58, 1.22 Hz, 1H), 9.25 (br s, 1H). LC-MS: (ES) m/z 576.3 (M+H⁺)) and (9R,10S)-10-[4-(cyclopentylamino)phenyl]-N-[4-methyl-3-(trifluoromethyl) phenyl]-12-oxo-6,6a,7,8,9,10-hexahydro-5H-pyrido[1,2-b][2]benzazepine-9-carboxamide (5 mg, 8.43 mol, 32.33% yield, 97% purity) was obtained as a white solid (¹H NMR (400 MHz, CDCl₃) δ 1.37 (dt, J=12.04, 6.33 Hz, 2H), 1.50-1.62 (m, 2H), 1.68-1.76 (m, 3H), 1.87-1.99 (m, 4H), 2.01-2.14 (m, 2H), 2.32 (s, 3H), 2.44-2.53 (m, 2H), 2.62-2.75 (m, 1H), 3.21 (dt, J=11.62, 5.93 Hz, 1H), 3.56 (br s, 1H), 3.65 (quin, J=6.24 Hz, 1H), 3.77 (br dd, J=12.84, 5.26 Hz, 1H), 6.36 (d, J=8.56 Hz, 2H), 6.83 (d, J=6.60 Hz, 1H), 6.98 (d, J=8.31 Hz, 1H), 7.07 (d, J=7.58 Hz, 1H), 7.22-7.26 (m, 1H), 7.31 (br dd, J=7.46, 1.10 Hz, 2H), 7.34-7.37 (m, 1H), 7.52 (d, J=8.56 Hz, 2H), 7.68 (dd, J=7.58, 1.22 Hz, 1H), 9.19 (br s, 1H). LC-MS: (ES) m/z 576.3 (M+H⁺)).

### Example S59: Synthesis of cis-4-(4-(cyclopentylamino)phenyl)-7-fluoro-N-(4-methyl-3-(trifluorometh yl)phenyl)-6-oxo-1,2,3,4,6,11,12,12a-octahydrobenzo[e]pyrido[1,2-a]azepine-3-carboxamide (Compound No. 41)

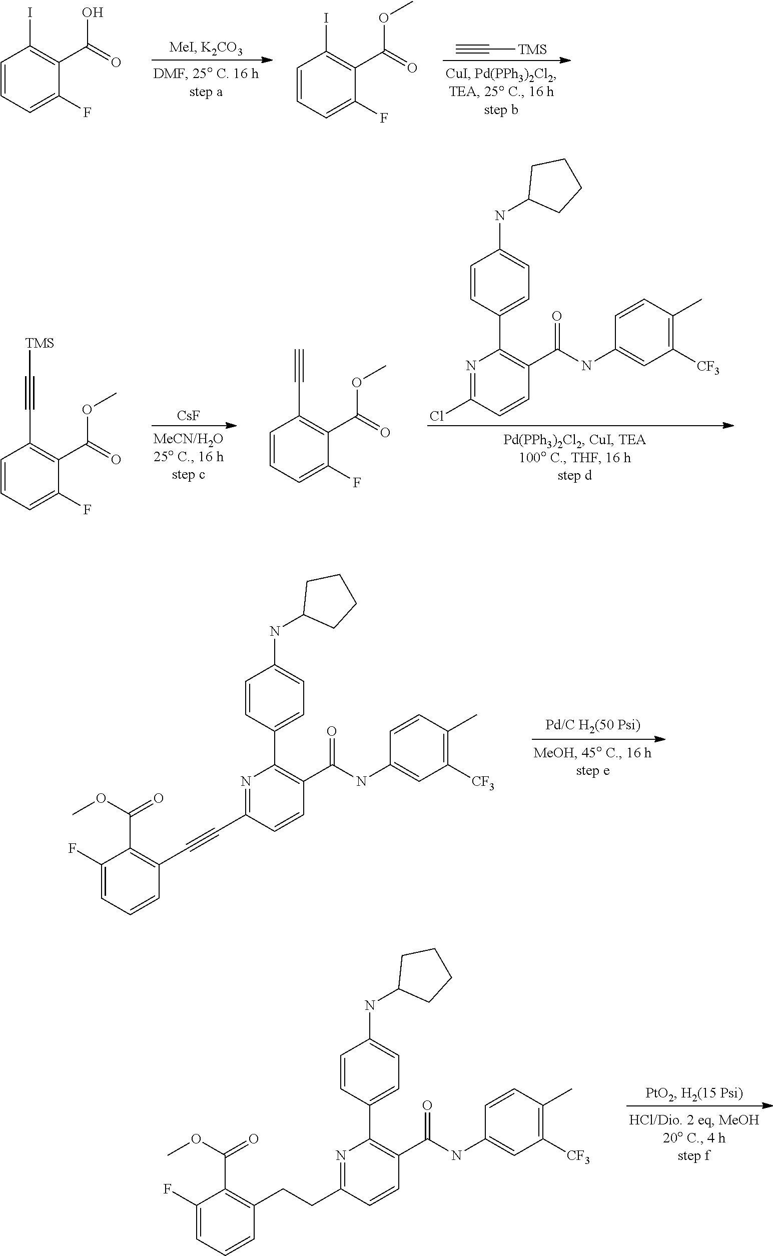

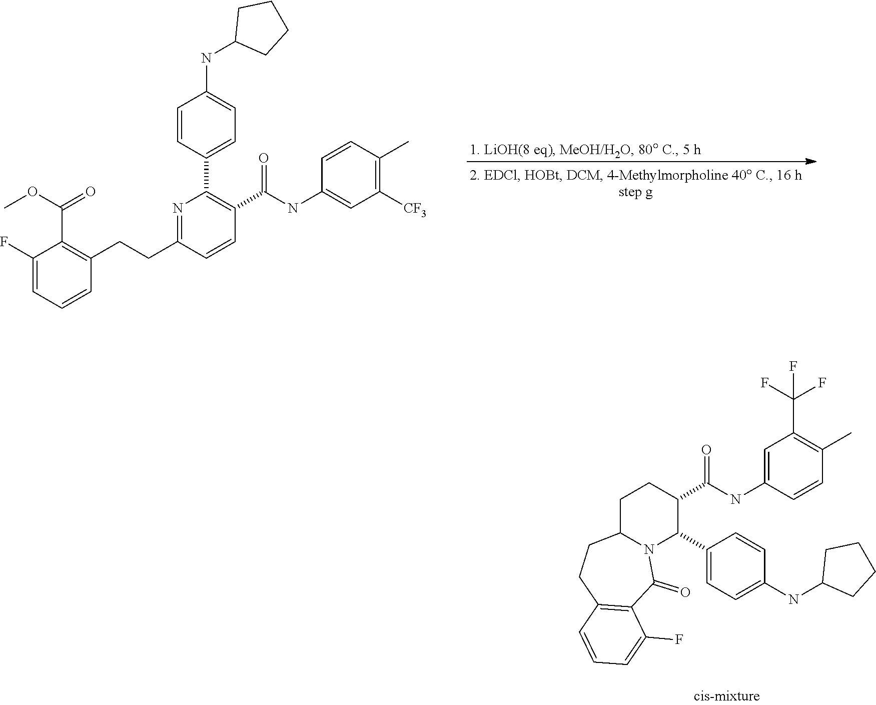

Step a) To a solution of 2-fluoro-6-iodo-benzoic acid (10 g, 37.59 mmol) in DMF (100 mL) was added K₂CO₃ (7.79 g, 56.39 mmol), then MeI (8.28 g, 58.33 mmol, 3.63 mL) was added. The mixture was stirred at 20° C. for 16 h. The reaction mixture was diluted with H₂O 100 mL and extracted with EtOAc 300 mL (150 mL×2). The combined organic layers were dried over anhydrous Na₂SO₄, filtered and concentrated under reduced pressure to give a residue. The residue was purified by column chromatography (SiO₂, petroleum ether/ethyl acetate=20/1) to give methyl 2-fluoro-6-iodo-benzoate (10.4 g, 36.40 mmol, 96.81% yield, 98% purity) as a colorless oil. ¹H NMR (400 MHz, CDCl₃) δ 3.99 (s, 3H), 7.09-7.16 (m, 2 H), 7.61-7.69 (m, 1H). LC-MS: (ES) m/z 280.8 (M+H⁺).

Step b) To a solution of methyl 2-fluoro-6-iodo-benzoate (11.1 g, 39.64 mmol) in TEA (80 mL) was added CuI (754.91 mg, 3.96 mmol) and Pd(PPh₃)₂Cl₂ (2.78 g, 3.96 mmol), then the ethynyl(trimethyl)silane (5.84 g, 59.46 mmol, 8.24 mL) in TEA (20 mL) was added by dropwise. The mixture was stirred at 20° C. for 16 h under N₂ atmosphere. The reaction mixture was diluted with H₂O 100 mL and extracted with EtOAc 500 mL (250 mL×2). The combined organic layers were dried over anhydrous Na₂SO₄, filtered and concentrated under reduced pressure to give a residue. The residue was purified by column chromatography (SiO₂, petroleum ether/ethyl acetate=20/1). The compound methyl 2-fluoro-6-(2-trimethylsilylethynyl) benzoate (9.9 g, 36.78 mmol, 92.78% yield, 93% purity) was obtained as a light brown oil. ¹H NMR (400 MHz, CDCl₃) δ 0.25 (s, 8H), 3.95 (s, 3H), 7.09 (ddd, J=9.35, 7.89, 1.59 Hz, 1H), 7.28-7.39 (m, 2H). LC-MS: (ES) m/z 251.1 (M+H⁺).

Step c) To a solution of methyl 2-fluoro-6-(2-trimethylsilylethynyl)benzoate (3 g, 11.98 mmol) in MeCN (80 mL) and H₂O (20 mL) was added CsF (7.28 g, 47.93 mmol, 1.77 mL). The mixture was stirred at 20° C. for 16 h. The reaction mixture was concentrated under reduced pressure to remove MeCN. The residue was extracted with ethyl acetate (250 mL×2). The combined organic layers were dried over anhydrate Na₂S04, filtered, and concentrated under reduced pressure to give a residue. The residue was purified by column chromatography (SiO₂, Petroleum ether/Ethyl acetate=30/1, plate 2) to give methyl 2-ethynyl-6-fluoro-benzoate (1.9 g, 10.56 mmol, 88.10% yield, 99% purity) as a light brown oil. ¹H NMR (400 MHz, CDCl₃) δ 3.29 (s, 1H), 3.97 (s, 3H), 7.10-7.17 (m, 1H), 7.33-7.43 (m, 2H). LC-MS: (ES) m/z 179.1 (M+H⁺).

Step d) To a solution of methyl 2-ethynyl-6-fluoro-benzoate (857.12 mg, 4.81 mmol) and 6-chloro-2-[4-(cyclopentylamino)phenyl]-N-[4-methyl-3-(trifluoromethyl)phenyl]pyridine-3-carboxamide (760 mg, 1.60 mmol) in THF (50 mL) was added CuI (30.54 mg, 160.37 mol), PPh₃ (42.06 mg, 160.37 mol) and TEA (2.55 g, 25.16 mmol, 3.50 mL), then the mixture was stirred at 20° C. for 3 min. To the mixture was added Pd(PPh₃)₂Cl₂ (112.56 mg, 160.37 μmol) and the mixture was heated at 100° C. for 16 h under N₂ atmosphere. The reaction mixture was concentrated under reduced pressure to remove THF. The residue was diluted with H₂O 100 mL and extracted with EtOAc (300 mL×2). The combined organic layers were dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜30% ethyl acetate/petroleum ether gradient@30 mL/min) to give 2-[2-[6-[4-(cyclopentylamino)phenyl]-5-[[4-methyl-3-(trifluoromethyl)phenyl]carbamoyl]-2-pyridyl]ethynyl]-3-fluoro-benzoate (720 mg, 1.09 mmol, 67.83% yield, 93% purity) as a brown solid. ¹H NMR (400 MHz, CDCl₃) δ 1.44-1.54 (m, 2H), 1.61-1.69 (m, 2H), 1.70-1.81 (m, 2H), 1.98-2.12 (m, 2H), 2.43 (s, 3H), 3.77-3.88 (m, 1H), 4.02 (s, 3H), 6.65 (d, J=8.56 Hz, 2H), 7.15-7.26 (m, 3H), 7.33 (s, 1H), 7.44 (td, J=8.01, 5.50 Hz, 2H), 7.48-7.55 (m, 4H), 8.19 (d, J=8.07 Hz, 1H). LC-MS: (ES) m/z 616.2 (M+H⁺).

Step e) To a solution of methyl 2-[2-[6-[4-(cyclopentylamino)phenyl]-5-[[4-methyl-3-(trifluoromethyl)phenyl]carbamoyl]-2-pyridyl]ethynyl]-3-fluoro-benzoate (700 mg, 1.14 mmol) in MeOH (30 mL) was added Pd/C (wet) (100 mg, 10% purity). The mixture was degassed and purged with H₂ (50 psi) 3 times, and then the mixture was stirred at 45° C. for 4 h under H₂ atmosphere. The residue was purified by flash silica gel chromatography (ISCO®; 12 g SepaFlash® Silica Flash Column, Eluent of 0˜30% ethyl acetate/petroleum ether gradient@35 mL/min) to give methyl 2-[2-[6-[4-(cyclopentylamino)phenyl]-5-[[4-methyl-3-(trifluoromethyl)phenyl]carbamoyl]-2-pyridyl]ethyl]-3-fluoro-benzoate (590 mg, 904.55 mol, 79.55% yield, 95% purity) as a yellow solid. ¹H NMR (400 MHz, CDCl₃) δ 1.43-1.53 (m, 2H), 1.62-1.69 (m, 2H), 1.71-1.80 (m, 2H), 2.00-2.05 (m, 2H), 2.42 (s, 3H), 3.19 (s, 4H), 3.69-3.77 (m, 1H), 3.79-3.88 (m, 1H), 3.94 (s, 3H), 6.66 (d, J=8.56 Hz, 2H), 6.99 (t, J=8.93 Hz, 1H), 7.07 (dd, J=17.12, 7.83 Hz, 2H), 7.16-7.24 (m, 2H), 7.28-7.36 (m, 2H), 7.42 (br d, J=8.31 Hz, 1H), 7.51 (d, J=8.56 Hz, 2H), 8.06 (d, J=8.07 Hz, 1H). LC-MS: (ES) m/z 620.3 (M+H⁺).

Step f) A mixture of methyl 2-[2-[6-[4-(cyclopentylamino)phenyl]-5-[[4-methyl-3-(trifluoromethyl)phenyl]carbamoyl]-2-pyridyl]ethyl]-3-fluoro-benzoate (580 mg, 936.02 mol), PtO₂ (106.28 mg, 468.01 mol) and HCl/dioxane (4 M, 470.24 μL) in MeOH (20 mL) was degassed and purged with H₂ (15 psi) for 3 times, and then the mixture was stirred at 20° C. for 4 hr under H₂ atmosphere. The reaction mixture was filtered and concentrated under reduced pressure to give a residue. The residue was alkalized with aqueous NaHCO₃ (10 ml) solution, then extracted with DCM 80 mL (40 mL×2). The combined organic layers were dried over anhydrous Na₂SO₄, filtered, and concentrated under reduced pressure to give a crude product. The crude product was purified by flash silica gel chromatography (ISCO®; 12 g SepaFlash® Silica Flash Column, eluent of 0˜2% ethyl acetate/petroleum ether gradient@35 mL/min) to give methyl 2-[2-[6-[4-(cyclopentylamino)-phenyl]-5-[[4-methyl-3-(trifluoromethyl)phenyl]carbamoyl]-2-piperidyl]ethyl]-3-fluoro-benzoate (520 mg, crude) as a brown gum. The crude product was further purified by prep-HPLC (HCl condition; column: Xtimate C18 150*40 mm*10 m; mobile phase: [water (0.05% HCl)-ACN]; B %: 30%-60%, 8 min) to give methyl 2-[2-[6-[4-(cyclopentylamino)phenyl]-5-[[4-methyl-3-(trifluoromethyl)phenyl]carbamoyl]-2-piperidyl]ethyl]-6-fluoro-benzoate (147 mg, 210.90 mol, 43.99% yield, 95% purity, HCl) as white solid. ¹H NMR (400 MHz, DMSO-d₆) δ 1.45 (br d, J=3.67 Hz, 2H), 1.57-1.78 (m, 6H), 1.85-2.05 (m, 2H), 2.12 (br s, 3H), 2.34 (br s, 3H), 2.65-2.87 (m, 2H), 3.22-3.36 (m, 2H), 3.73-3.81 (m, 2H), 3.90 (s, 3H), 4.67 (br d, J=9.78 Hz, 1H), 7.18-7.35 (m, 4H), 7.46-7.61 (m, 4H), 7.98 (s, 1H), 8.36 (br d, J=10.76 Hz, 1H), 9.71 (br s, 1H), 10.85 (s, 1H). LC-MS: (ES) m/z 626.3 (M+H⁺).

Step g) To a solution of methyl 2-[2-[6-[4-(cyclopentylamino)phenyl]-5-[[4-methyl-3-(trifluoromethyl)phenyl]carbamoyl]-2-piperidyl]ethyl]-6-fluoro-benzoate (150 mg, 239.73 mol) in MeOH (3 mL) and H₂O (1 mL) was added LiOH (45.93 mg, 1.92 mmol). The mixture was stirred at 80° C. for 4 h. The reaction was concentrated and re-dissolved in DCM (5 mL). Then EDCI (137.87 mg, 719.20 mol), HOBt (32.39 mg, 239.73 mol) and 4-methylmorpholine (92.14 mg, 910.99 mol, 100.16 μL) were added and the mixture was stirred at 40° C. for 16 h. The mixtures were concentrated under reduced pressure to remove DCM. The residue was diluted with H₂O 10 mL and extracted with DCM 50 mL (25 mL×2). The combined organic layers were dried over anhydrous Na₂SO₄, filtered and concentrated under reduced pressure to give a residue. The crystallized solid was collected after re-crystallization from MeCN. The crystal was washed with MeCN 2 mL, filtered and the filter cake was dried under vacuum to give 10-[4-(cyclopentylamino)phenyl]-1-fluoro-N-[4-methyl-3-(trifleoromethyl)phenyl]-12-oxo-6,6a,7,8,9,10-hexahydro-5H-pyrido[1,2-b][2]benzazepine-9-carboxamide (45 mg, 74.29 μmol, 30.99% yield, 98% purity) as a white solid. ¹H NMR (400 MHz, DMSO-d₆) δ 1.35 (br s, 2H), 1.44-1.55 (m, 2H), 1.56-1.68 (m, 3H), 1.70-1.91 (m, 5H), 2.08-2.18 (m, 1H), 2.21-2.33 (m, 2H), 2.34 (br s, 3H), 2.53-2.61 (m, 2H), 2.89-2.98 (m, 1H), 3.57 (sxt, J=6.11 Hz, 1H), 3.65-3.76 (m, 1H), 5.47 (d, J=6.36 Hz, 1H), 6.07 (d, J=7.09 Hz, 1H), 6.35 (d, J=8.56 Hz, 2H), 7.00 (d, J=7.34 Hz, 1 H), 7.13 (t, J=9.17 Hz, 1H), 7.21 (d, J=8.56 Hz, 2H), 7.29 (d, J=8.31 Hz, 1H), 7.38 (td, J=7.89, 5.75 Hz, 1H), 7.50 (br d, J=8.07 Hz, 1H), 7.76 (d, J=1.71 Hz, 1H), 10.23 (s, 1H). LC-MS: (ES) m/z 594.4 (M+H⁺).

### Example S60: Synthesis of cis-2-(4-(cyclopentylamino)phenyl)-1-(2-fluoro-6-methylbenzoyl)-N-(4-methyl-3-(trifluoromethyl)phenyl)octahydro-1H-cyclopenta[b]pyridine-3-carboxamide (Compound No. 193)

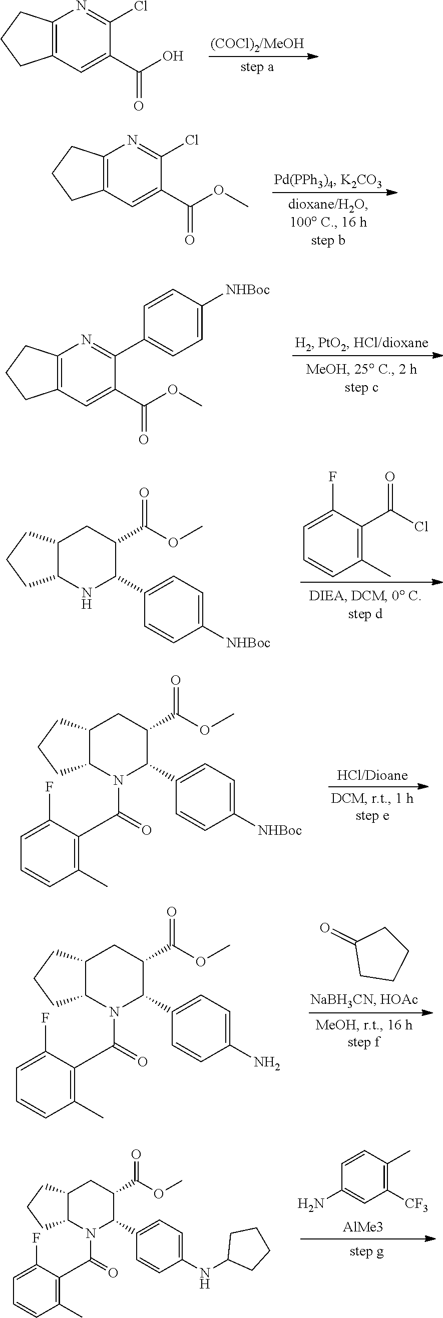

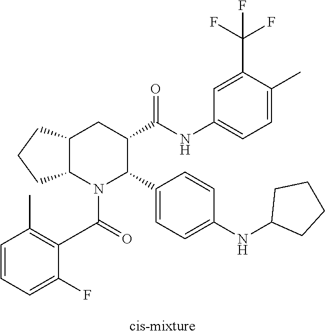 [SMILES: Cc1ccc(NC(=O)[C@H]2C[C@H]3CCC[C@H]3N(C(=O)c3c(C)cccc3F)[C@H]2c2ccc(NC3CCCC3)cc2)cc1C(F)(F)F]

Step a) To a mixture of 2-chloro-6,7-dihydro-5H-cyclopenta[b]pyridine-3-carboxylic acid (485 mg, 2.45 mmol) in DCM (20 mL) was added oxalyl dichloride (467.26 mg, 3.68 mmol, 322.25 μL) and DMF (17.94 mg, 245.42 mol, 18.88 μL). Then the mixture was stirred at 25° C. for 15 min. The solvent was evaporated under vacuum. Then methanol (7.92 g, 247.12 mmol, 10 mL) was added. The reaction mixture was stirred at 25° C. for another 15 min. The solvent was evaporated under vacuum to give methyl 2-chloro-6,7-dihydro-5H-cyclopenta[b]pyridine-3-carboxylate (500 mg, crude) as a brown oil. LC-MS: (ES) m/z 212 (M+H⁺).

Step b) Pd(PPh₃)₄ (545.99 mg, 472.49 mol) was added to a mixture of methyl 2-chloro-6,7-dihydro-5H-cyclopenta[b]pyridine-3-carboxylate (500 mg, 2.36 mmol), [4-(tert-butoxycarbonyl amino)phenyl]boronic acid (840.06 mg, 3.54 mmol) and K₂CO₃ (979.54 mg, 7.09 mmol) in dioxane/H₂O=1:1 (20 mL). The mixture was stirred at 100° C. under N₂ for 3 h. The reaction mixture was extracted with EtOAc (30 mL×2). The combined organic phase were washed with brine (30 mL), dried with anhydrous MgSO₄ and filtered. The filtrate was evaporated under vacuum to a residue. The residue was purified by column chromatography (SiO₂, petroleum ether/ethyl acetate=100/0 to 3:1) to give methyl 2-[4-(tert-butoxycarbonylamino)phenyl]-6,7-dihydro-5H-cyclopenta[b]pyridine-3-carboxylate (480 mg, crude) as a white solid. LC-MS: (ES) m/z 369.2 (M+H⁺).

Step c) PtO₂ (11.83 mg, 52.11 mol) was added to a solution of methyl 2-[4-(tert-butoxycarbonylamino)phenyl]-6,7-dihydro-5H-cyclopenta[b]pyridine-3-carboxylate (480 mg, 1.30 mmol) and HCl (in H₂O) (12 M, 217.14 μL) in EtOH (10 mL). Then the mixture was stirred at 25° C. under H₂ (15 psi) for 16 h. The reaction mixture was filtered. The filtrate was evaporated under vacuum. Then the mixture was added 10 mL of H₂O, alkalified with Na₂CO₃ solution and extracted with EtOAc (30 mL×2). The combined organic phase were washed with brine (20 mL), dried with anhydrous MgSO₄ and filtered. The filtrate was evaporated under vacuum to give cis-methyl 2-[4-(tert-butoxycarbonylamino)phenyl]-2,3,4, 4a,5,6,7,7a-octahydro-1H-cyclopenta[b]pyridine-3-carboxylate (450 mg, 997.40 mol, 76.56% yield, 83% purity) as yellow oil. ¹H NMR (400 MHz, DMSO-d₆) δ 1.35-1.52 (12 H, m) 1.54-1.90 (6H, m) 2.00-2.09 (1H, m) 2.81-2.97 (1H, m) 3.15 (1H, brd, J=5.62 Hz) 3.21-3.29 (3H, m) 3.86 (1H, br d, J=5.14 Hz) 7.16 (2H, br d, J=8.07 Hz) 7.23-7.37 (2H, m) 9.19 (1 H, br s). LC-MS: (ES) m/z 375.2 (M+H⁺).

Step d) 2-fluoro-6-methyl-benzoyl chloride (172.14 mg, 997.40 mol) was added to a solution of cis-methyl-2-[4-(tert-butoxycarbonylamino)phenyl]-2,3,4,4a,5,6,7,7a-octahydro-1H-cyclopenta[b]pyridine-3-carboxylate (450.00 mg, 997.40 mol) and TEA (201.85 mg, 1.99 mmol, 277.65 μL) in DCM (10 mL). The mixture was stirred at 25° C. for 1 h. The reaction mixture was washed with 1N HCl (10 mL), H₂O (10 mL), brine (10 mL), dried with anhydrous Na₂SO₄ and filtered. The filtrate was evaporated under vacuum to give a residue. The residue was purified by column chromatography (SiO₂, petroleum ether/ethyl acetate=100/0 to 3:1) to give cis-methyl 2-[4-(tert-butoxycarbonylamino)phenyl]-1-(2-fluoro-6-methyl-benzoyl)-2,3,4,4a,5,6,7,7a-octahydro-cyclopenta[b]pyridine-3-carboxylate (440 mg, 766.95 mol, 76.89% yield, 89% purity) as a white solid. LC-MS: (ES) m/z 511.2 (M+H⁺).

Step e) HCl/dioxane (4 M, 215.43 μL) was added to a solution of cis-methyl 2-[4-(tert-butoxycarbonylamino)phenyl]-1-(2-fluoro-6-methyl-benzoyl)-2,3,4,4a,5,6,7,7a-octahydro-cyclopenta[b]pyridine-3-carboxylate (440 mg, 766.95 mol) in DCM (10 mL). Then the solution was stirred at 25° C. for 1 h. The solvent was evaporated under vacuum to give cis-methyl 2-(4-amino phenyl)-1-(2-fluoro-6-methyl-benzoyl)-2,3,4,4a,5,6,7,7a-octahydrocyclopenta[b]pyridine-3-carboxylate (390 mg, crude, HCl) as a brown oil. LC-MS: (ES) m/z 411.2 (M+H⁺).

Step f) To cyclopentanone (73.40 mg, 872.60 μmol, 77.26 μL) in DCM (10 mL) was added cis-methyl2-(4-aminophenyl)-1-(2-fluoro-6-methyl-benzoyl)-2,3,4,4a,5,6,7,7a-octahydro-cyclopenta[b]pyridine-3-carboxylate (358.18 mg, 872.60 mol, HCl), CH₃COOH (157.20 mg, 2.62 mmol, 149.71 μL) and HCl/dioxane (4 M, 283.59 μL), followed by NaBH(OAc)₃ (277.41 mg, 1.31 mmol). The mixture was stirred at 25° C. for 16 h. The reaction mixture was basified with Na₂CO₃ solution and extracted with DCM (30 mL×2). The combined organic layers were washed with brine (30 mL), dried over anhydrous Na₂SO₄, filtered, and concentrated under reduced pressure to give crude product. The crude product was purified by prep-HPLC (column: Agela ASB 150×25 mm×5 μm; mobile phase: [water (0.05% HCl)-ACN]; B %: 42%-72%, 8 min) to give cis-methyl-2-[4-(cyclopentylamino)phenyl]-1-(2-fluoro-6-methyl-benzo yl)-2,3,4,4a,5,6,7,7a-octahydrocyclopenta[b]pyridine-3-carboxylate (200 mg, 417.89 mol, 47.89% yield, 100% purity) as a light yellow solid. ¹H NMR (400 MHz, DMSO-d₆) δ 0.96-1.21 (3H, m), 1.23-1.46 (4H, m), 1.47-1.59 (3H, m), 1.59-1.72 (3H, m), 1.74-1.95 (3H, m), 1.95-2.11 (2H, m), 2.23-2.36 (3H, m), 2.88-3.04 (1H, m), 3.50-3.71 (4H, m), 5.53-5.61 (1H, m), 6.38-6.44 (1 H, m), 6.48 (2H, dd, J=8.91, 2.38 Hz), 6.99 (1H, d, J=8.53 Hz), 7.04-7.16 (2H, m), 7.30-7.40 (1H, m). LC-MS: (ES) m/z 479.2 (M+H⁺).

Step g) AlMe₃ (in toluene) (2 M, 156.71 μL) was added to a solution of 4-methyl-3-(trifluoromethyl)aniline (82.34 mg, 470.12 mol) in DCE (6 mL). The mixture was stirred at 25° C. for 20 min. Then cis-methyl-2-[4-(cyclopentylamino)phenyl]-1-(2-fluoro-6-methyl-benzoyl)-2,3,4,4a,5,6,7,7a-octahydrocyclopenta[b]pyridine-3-carboxylate (150 mg, 313.42 mol) was added to the mixture. The mixture was stirred at 85° C. for 3 h. The reaction mixture was basified with saturate NaHCO₃ solution. Then the mixture was extracted with EtOAc (50 mL×2). The combined organic phase was washed with brine (50 mL), dried with anhydrous MgSO₄ and was filtered. The filtrate was evaporated under vacuum to give crude product. The crude product was purified by prep-HPLC (column: Xtimate C18 10μ250 mm×50 mm; mobile phase: [water (0.04% NH₃H₂O+10 mM NH₄HCO₃)-ACN]; B %: 80%-100%, 8 min), then further purified by prep-HPLC (column: Agela ASB 150×25 mm×5 μm; mobile phase: [water (0.05% HCl)-ACN]; B %: 55%-85%, 8 min) to give cis-2-[4-(cyclopentylamino)phenyl]-1-(2-fluoro-6-methyl-benzoyl)-N-[4-methyl-3-(trifluoromethyl)phenyl]-2,3,4,4a,5,6,7,7a-octahydrocyclopenta[b]pyridine-3-carboxamide (58 mg, 92.36 umol, 99% purity) as a white solid. (¹H NMR (400 MHz, DMSO-d₆) δ 1.07-1.30 (3H, m) 1.50-1.61 (5H, m) 1.63-1.76 (4H, m) 1.88 (3H, br d, J=7.28 Hz) 1.93-2.08 (2H, m) 2.09-2.19 (2H, m) 2.30-2.44 (6H, m) 2.99-3.09 (1H, m) 3.60-3.78 (1H, m) 6.47-6.59 (1H, m) 6.90-7.00 (2H, m) 7.05-7.18 (2H, m) 7.28-7.41 (2H, m) 7.52 (2 H, dd, J=16.56, 8.53 Hz) 7.64-7.75 (1H, m) 7.89 (1H, dd, J=14.81, 1.76 Hz) 10.15 (1 H, br d, J=8.78 Hz). LC-MS: (ES) m/z 622.3 (M+H⁺).

### Example S61: Synthesis of (2R,3S,4aR,7aR)-2-(4-(cyclopentylamino)phenyl)-1-(2-fluoro-6-methylbenzoyl)-N-(4-methyl-3-(trifluoromethyl)phenyl)octahydro-1H-cyclopenta[b]pyridine-3-carboxamide (Compound No. 48)

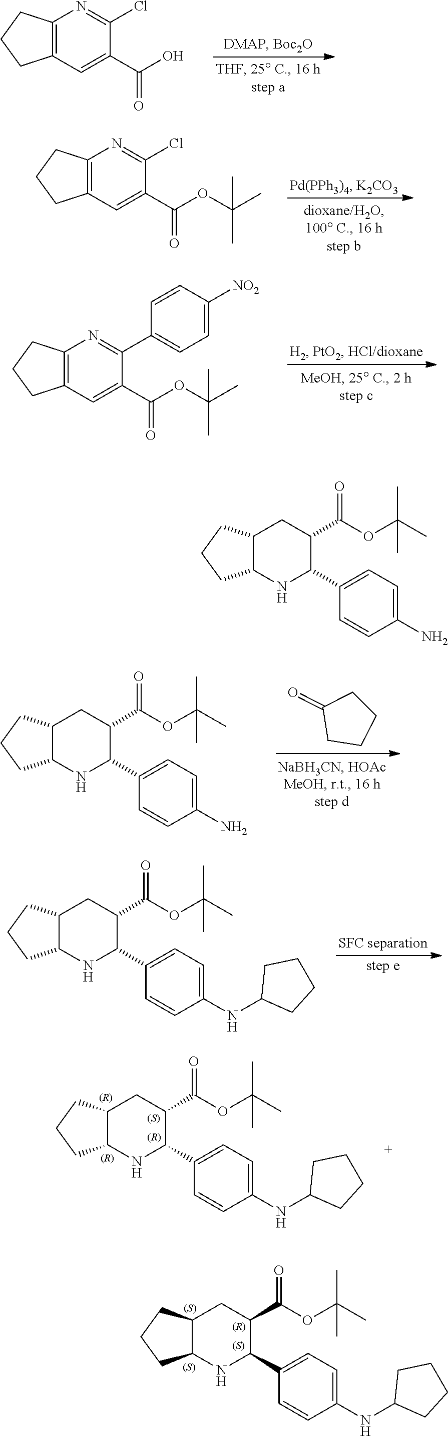

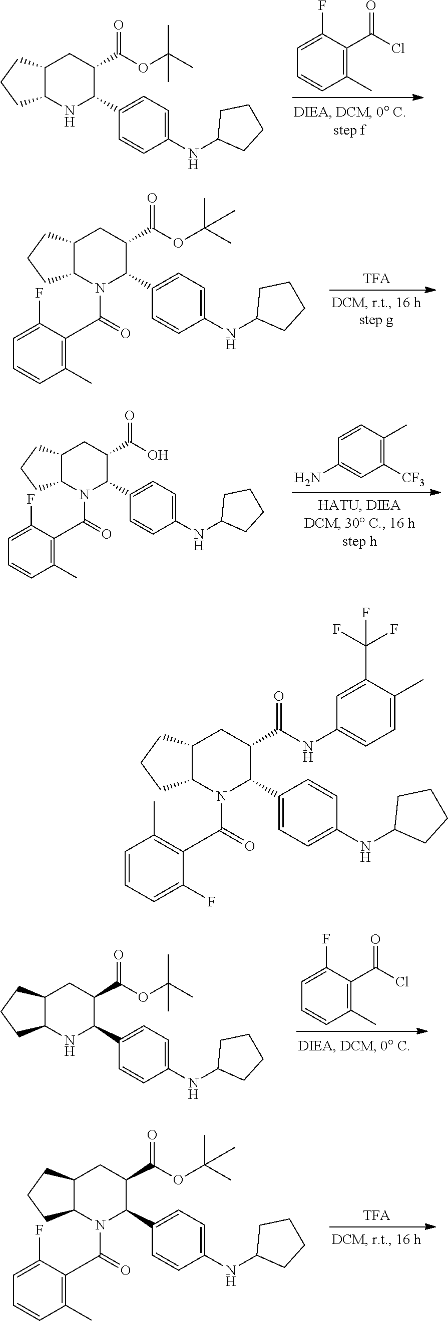

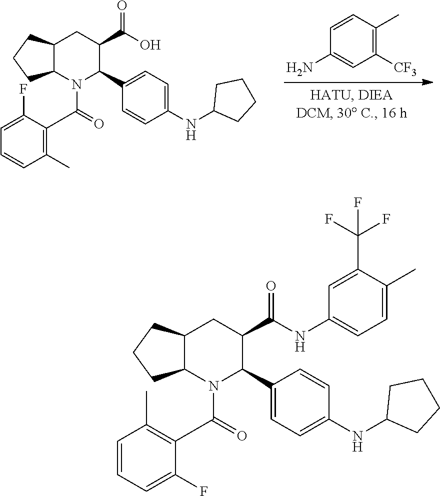 [SMILES: Cc1cccc(F)c1C(=O)N1[C@H](c2ccc(NC3CCCC3)cc2)[C@H](C(=O)O)C[C@@H]2CCC[C@@H]21 ; Cc1ccc(N)cc1C(F)(F)F ; Cc1ccc(NC(=O)[C@@H]2C[C@@H]3CCC[C@@H]3N(C(=O)c3c(C)cccc3F)[C@@H]2c2ccc(NC3CCCC3)cc2)cc1C(F)(F)F]

Step a) The DMAP (247.29 mg, 2.02 mmol) was added to a solution of 2-chloro-6,7-dihydro-5H-cyclopenta[b]pyridine-3-carboxylic acid (800 mg, 4.05 mmol) and tert-butoxy carbonyl tert-butyl carbonate (1.77 g, 8.10 mmol, 1.86 mL) in THF (20 mL). The solution was stirred at 15° C. for 16 h. The reaction mixture was extracted with EtOAc (30 mL×2). The combined organic phase was dried with anhydrous Na₂SO₄ and filtered. The filtrate was evaporated under vacuum to give residue. The residue was purified by column chromatography (SiO₂, petroleum ether/ethyl acetate=100:0 to 3:1) to give compound tert-butyl 2-chloro-6,7-dihydro-5H-cyclopenta[b]pyridine-3-carboxylate (900 mg, 3.55 mmol, 87.62% yield, 100% purity) as white solid. ¹H NMR (400 MHz, CDCl₃) δ 1.58 (9 H, s), 2.15 (2 H, quin, J=7.58 Hz), 2.92 (2H, t, J=7.46 Hz), 3.00 (2H, t, J=7.70 Hz), 7.83 (1 H, s). LC-MS: (ES) m/z 254.0 (M+H⁺).

Step b) Pd(PPh₃)₄ (409.90 mg, 354.72 mol) was added to a mixture of tert-butyl 2-chloro-6,7-dihydro-5H-cyclopenta[b]pyridine-3-carboxylate (900.00 mg, 3.55 mmol), (4-nitrophenyl)boronic acid (769.75 mg, 4.61 mmol) and K₂CO₃ (1.47 g, 10.64 mmol) in dioxane/H₂O=1:1 (30 mL). The mixture was stirred at 100° C. under N₂ for 16 h. The reaction mixture was extracted with EtOAc (30 mL×2). The combined organic phase were washed with brine (30 mL), dried with anhydrous MgSO₄ and filtered. The filtrate was evaporated under vacuum to a residue. The residue was purified by column chromatography (SiO₂, petroleum ether/ethyl acetate=100/0 to 3:1) to give tert-butyl 2-(4-nitrophenyl)-6,7-dihydro-5H-cyclopenta[b] pyridine-3-carboxylate (935 mg, 2.75 mmol, 77.44% yield, 100% purity) as a yellow solid. ¹H NMR (400 MHz, CDCl₃) δ 1.29 (9 H, s), 2.20 (2 H, quin, J=7.46 Hz), 2.98-3.11 (4H, m), 7.63 (2H, br d, J=7.58 Hz), 7.94 (1 H, s), 8.27 (2H, br d, J=7.58 Hz). LC-MS: (ES) m/z 341.1 (M+H⁺).

Step c) To a solution of tert-butyl 2-(4-nitrophenyl)-6,7-dihydro-5H-cyclopenta [b] pyridine-3-carboxylate (1.1 g, 3.23 mmol) in MeOH (30 mL) was added PtO₂ (366.93 mg, 1.62 mmol) and HCl/dioxane (4 M, 1.62 mL) under N₂. The suspension was degassed under vacuum and purged with H₂ several times. The mixture was stirred under H₂ (15 psi) at 20° C. for 2 hours. LCMS showed ˜80% of desired product was detected. The mixture was diluted with MeOH and filtered through a pad of Celite and concentrated in vacuo to give the residue. The residue was diluted with DCM (50 mL) and alkalified to pH=9˜10. The organic layers separated was dried, filtered and concentrated in vacuo to give the residue. The crude residue was purified by column chromatography (SiO₂, eluted with DCM/MeOH/NH₃·H₂O=100/1/0.01 to 10/1/0.01) to give cis-tert-butyl 2-(4-aminophenyl)-2,3,4,4a,5,6,7,7a-octahydro-1H-cyclopenta[b]pyridine-3-carboxylate (0.6 g, 1.90 mmol, 66.67% yield, 100% purity) as light brown gum. ¹H NMR (400 MHz, CDCl₃) δ 1.18 (s, 9H), 1.49-1.65 (m, 3H), 1.68-1.82 (m, 2H), 1.83-1.94 (m, 1H), 1.99-2.09 (m, 2H), 2.10-2.19 (m, 1H), 2.80 (q, J=6.02 Hz, 1H), 3.34 (td, J=6.34, 2.89 Hz, 1H), 3.45-3.66 (m, 2H), 3.93 (d, J=5.52 Hz, 1H), 6.63 (d, J=8.53 Hz, 2H), 7.15 (d, J=8.28 Hz, 2H). LC-MS: (ES) m z 317.2 (M+H⁺).

Step d) To a mixture of cis-tert-butyl 2-(4-aminophenyl)-2,3,4,4a,5,6,7,7a-octahydro-1H-cyclopenta[b]pyridine-3-carboxylate (520.00 mg, 1.64 mmol) in MeOH (10 mL) was added cyclopentanone (179.70 mg, 2.14 mmol, 189.16 μL), HOAc (197.36 mg, 3.29 mmol, 187.96 μL) and NaBH₃CN (516.32 mg, 8.22 mmol) in one portion at 0° C. under N₂. The mixture was stirred at 30° C. for 16 h. The mixture was diluted with DCM (30 mL) and alkalified to pH=8-9 and extracted with DCM (3×30 mL). The combined organic layers were washed with brine, dried, filtered and concentrated in vacuo to give the desired compound tert-butyl 2-[4-(cyclopentylamino)phenyl]-2,3,4,4a,5,6,7,7a-octahydro-1H-cyclopenta[b]pyridine-3-carboxylate (0.6 g, 1.50 mmol, 91.15% yield, 96% purity) was obtained as light brown gum. ¹H NMR (400 MHz, CDCl₃) δ 1.10-1.24 (m, 9H), 1.39-1.47 (m, 2H), 1.58-1.64 (m, 2H), 1.66-1.77 (m, 4H), 1.82-1.90 (m, 2H), 1.98-2.11 (m, 6 H), 2.16-2.22 (m, 1H), 2.78 (q, J=6.11 Hz, 1H), 3.27-3.35 (m, 1H), 3.78 (quin, J=6.17 Hz, 1H), 3.92 (d, J=5.62 Hz, 1H), 6.54 (d, J=8.31 Hz, 2H), 7.13 (d, J=8.31 Hz, 2H). LC-MS: (ES) m/z 385.3 (M+H⁺).

Step e) The racemate cis-tert-butyl 2-[4-(cyclopentylamino)phenyl]-2,3,4,4a,5,6,7,7a-octahydro-1H-cyclopenta[b]pyridine-3-carboxylate (850.00 mg, 2.21 mmol) was separated by SFC. (column: REGIS (s, s) WHELK-O1 (250 mm×30 mm, 5 m); mobile phase: [0.1% NH₃·H₂O ETOH]; B %: 30%-30%, 8 min). The compound tert-butyl (2S,3R,4aS,7aS)-2-[4-(cyclopentyl amino)phenyl]-2,3,4,4a,5,6,7,7a-octahydro-1H-cyclopenta [b] pyridine-3-carboxylate (peak 1 showed on SFC spectrum, 0.37 g, 923.67 mol, 41.79% yield, 96% purity) was obtained as light yellow gum. ¹H NMR (400 MHz, CDCl₃) δ 1.16 (s, 9H), 1.36-1.47 (m, 2H), 1.48-1.64 (m, 5H), 1.67-1.81 (m, 4H), 1.82-1.89 (m, 1H), 1.94-2.06 (m, 4H), 2.07-2.16 (m, 1H), 3.78 (quin, J=6.24 Hz, 1H), 3.91 (d, J=5.87 Hz, 1H), 5.31 (s, 1H), 6.54 (d, J=8.56 Hz, 2H), 7.13 (d, J=8.31 Hz, 2H). LC-MS: (ES) m/z 385.3 (M+H⁺). The compound tert-butyl (2R,3S,4aR,7aR)-2-[4-(cyclopentylamino)phenyl]-2,3,4,4a,5,6,7,7a-octahydro-1H-cyclopenta[b] pyridine-3-carboxylate (peak 2 showed on SFC spectrum, 0.39 g, 953.31 mol, 43.13% yield, 94% purity) was obtained as light yellow gum. ¹H NMR (400 MHz, CHLOROFORM-d) δ 1.17 (s, 9H), 1.36-1.45 (m, 2H), 1.50-1.63 (m, 5H), 1.67-1.81 (m, 4H), 1.83-1.92 (m, 1H), 1.95-2.07 (m, 4H), 2.08-2.16 (m, 1H), 2.78 (q, J=6.11 Hz, 1H), 3.30 (dt, J=6.48, 3.36 Hz, 1H), 3.78 (quin, J=6.17 Hz, 1H), 3.92 (d, J=5.87 Hz, 1H), 6.54 (d, J=8.31 Hz, 2H), 7.14 (d, J=8.31 Hz, 2H). LC-MS: (ES) m/z 385.3 (M+H⁺).

Step f) To a solution of tert-butyl(2S,3R,4aS,7aS)-2-[4-(cyclopentylamino)phenyl]-2,3,4,4a,5,6,7,7a-octahydro-1H-cyclopenta[b]pyridine-3-carboxylate (0.25 g, 650.10 mol) and DIEA (168.04 mg, 1.30 mmol, 226.47 μL) in DCM (10 mL) was added dropwise of a solution of 2-fluoro-6-methyl-benzoyl chloride (106.59 mg, 617.60 mol) in DCM (3 mL) at 0° C. The mixture was stirred at 0° C. for 10 min. The mixture was diluted with DCM (10 mL), washed with H₂O (2×2 mL), dried, filtered and concentrated in vacuo to give the crude product. The crude product was purified by flash silica gel chromatography (ISCO®; 12 g SepaFlash® Silica Flash Column, eluent of 0˜20% ethyl acetate/petroleum ether gradient@30 mL/min) to give the target compound tert-butyl (2R,3S,4aR,7aR)-2-[4-(cyclopentylamino) phenyl]-1-(2-fluoro-6-methyl-benzoyl)-2,3,4,4a,5,6,7,7a-octahydrocyclopenta[b]pyridine-3-carb oxylate (100% purity) as white solid. ¹H NMR (400 MHz, DMSO-d₆) δ 1.06-1.21 (m, 4H), 1.26-1.37 (m, 9H), 1.38-1.46 (m, 2H), 1.47-1.57 (m, 3H), 1.59-1.69 (m, 2H), 1.71-2.05 (m, 6H), 2.18-2.35 (m, 3H), 2.75-2.93 (m, 1H), 3.47-3.71 (m, 2H), 5.52 (br d, J=6.27 Hz, 1H), 6.29-6.40 (m, 1 H), 6.43-6.53 (m, 2H), 6.98-7.21 (m, 4H), 7.33 (q, J=7.45 Hz, 1H). LC-MS: (ES) m z 521.3 (M+H⁺).

Step g) To a solution of tert-butyl(2R,3S,4aR,7aR)-2-[4-(cyclopentylamino)phenyl]-1-(2-fluoro-6-methyl-benzoyl)-2,3,4,4a,5,6,7,7a-octahydrocyclopenta[b]pyridine-3-carboxylate (0.28 g, 537.76 mol) in DCM (6 mL) was added TFA (2.31 g, 20.26 mmol, 1.50 mL) at 10° C. The mixture was stirred at 25° C. for 16 h. The mixture was concentrated in vacuo to give the residue. HCl/dioxane (4 M, 1 mL) was added to residue and the mixture was concentrated in vacuo to give the crude. The crude was triturated with MTBE (6 mL) at 15° C. for 0.5 h. The suspension was filtered. The filter cake was dried under vacuum to give the pure product (2R,3S,4aR,7aR)-2-[4-(cyclopentylamino)phenyl]-1-(2-fluoro-6-methyl-benzoyl)-2,3,4,4a,5,6,7,7a-octahydrocyclopenta-[b]pyridine-3-carboxylic acid (0.23 g, 485.18 mol, 63.16% yield, 98% purity) as off-white solid. ¹H NMR (400 MHz, CDCl₃) δ 0.67 (br s, 1H), 1.14 (br s, 1H), 1.37 (br d, J=10.76 Hz, 2H), 1.61-1.69 (m, 2H), 1.74-2.19 (m, 11H), 2.28-2.37 (m, 3 H), 2.85-3.07 (m, 1H), 3.56-3.80 (m, 3H), 6.54-6.65 (m, 1H), 6.90-6.99 (m, 1H), 7.04 (d, J=7.58 Hz, 1H), 7.30 (br s, 1H), 7.36-7.46 (m, 4H). LC-MS: (ES) m/z 465.2 (M+H⁺).

Step h) A mixture of (2R,3S,4aR,7aR)-2-[4-(cyclopentylamino)phenyl]-1-(2-fluoro-6-methyl-benzoyl)-2,3,4,4a,5,6,7,7a-octahydrocyclopenta[b]pyridine-3-carboxylic acid (0.1 g, 215.25 mol), HATU (98.21 mg, 258.30 μmol) and DIEA (69.55 mg) in DCM (2 mL) was stirred at 10° C. for 0.5 h. Then 4-methyl-3-(trifluoromethyl)aniline (45.24 mg, 258.30 mol, 37.08 μL) was added and the mixture was stirred at 30° C. for another 16 h. The mixture from the batch (0.12 g) were combined with this batch. The combined mixture was diluted with DCM (10 mL), washed with HCl (1 M, 2×0.25 mL), and then alkalified to pH=8-9 by addition of saturated NaHCO₃ solution. The organic layer was dried, filtered and concentrated in vacuo to give the crude. The crude was purified by flash silica gel chromatography (ISCO®; 12 g SepaFlash® Silica Flash Column, eluent of 0˜16% ethyl acetate/petroleum ether gradient@30 mL/min) to give target compound (2R,3S,4aR,7aR)-2-[4-(cyclopentylamino) phenyl]-1-(2-fluoro-6-methyl-benzoyl)-N-[4-methyl-3-(trifluoromethyl)phenyl]-2,3,4,4a,5,6,7,7a-octahydrocyclopenta[b]pyridine-3-carboxamide (160 mg, 98% purity) as white solid. ¹H NMR (400 MHz, DMSO-d₆) δ 1.09-1.26 (m, 2H), 1.28-1.43 (m, 4H), 1.46-1.55 (m, 3H), 1.56-1.69 (m, 3H), 1.79-1.98 (m, 4H), 2.04 (br d, J=8.03 Hz, 1H), 2.18 (s, 1H), 2.30 (s, 2H), 2.33-2.40 (m, 3H), 2.89-2.98 (m, 1H), 3.53-3.74 (m, 2H), 5.46-5.53 (m, 1H), 6.41 (d, J=8.53 Hz, 2H), 6.44-6.53 (m, 1H), 7.07-7.17 (m, 2H), 7.25 (d, J=8.78 Hz, 1H), 7.29-7.41 (m, 3H), 7.65-7.80 (m, 1H), 7.92 (dd, J=8.78, 2.01 Hz, 1H), 10.34 (d, J=13.55 Hz, 1H). LC-MS: (ES) m/z 622.3 (M+H⁺).

### Example S62: Synthesis of (2S,3R,4aS,7aS)-2-(4-(cyclopentylamino)phenyl)-1-(2-fluoro-6-methylbenzoyl)-N-(4-methyl-3-(trifluoromethyl)phenyl)octahydro-1H-cyclopenta[b]pyridine-3-carboxamide (Compound No. 47)

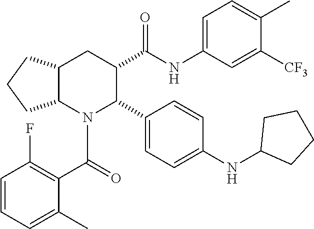

The title compound was synthesized in similar fashion as Example S62. ¹H NMR (400 MHz, DMSO-d₆) δ 1.01-1.24 (m, 2H), 1.25-1.42 (m, 4H), 1.43-1.54 (m, 3H), 1.55-1.67 (m, 3H), 1.78-1.96 (m, 4H), 1.99-2.08 (m, 1H), 2.17 (s, 1H), 2.27-2.32 (m, 2H), 2.32-2.38 (m, 3H), 2.88-3.01 (m, 1H), 3.53-3.73 (m, 2H), 5.51 (br s, 1H), 6.41 (d, J=8.28 Hz, 2H), 6.47 (dd, J=12.80, 5.77 Hz, 1H), 7.05-7.17 (m, 2H), 7.24 (d, J=8.78 Hz, 1 H), 7.28-7.39 (m, 3H), 7.66-7.79 (m, 1H), 7.91 (dd, J=8.78, 2.01 Hz, 1H), 10.34 (d, J=13.55 Hz, 1H). LC-MS: (ES) m/z 622.3 (M+H⁺).

### Example S63: Synthesis of cis-2-(4-(cyclopentylamino)phenyl)-1-(2-fluoro-6-methylbenzoyl)-N-(1-methyl-1H-indazol-5-yl)octahydro-1H-cyclopenta[b]pyridine-3-carboxamide (Compound No. 147)

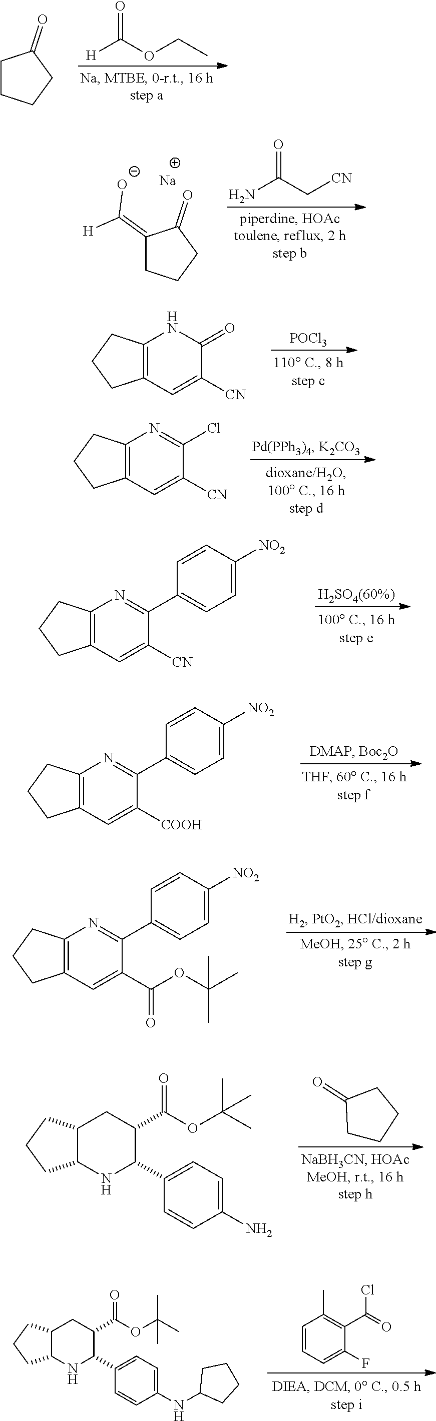

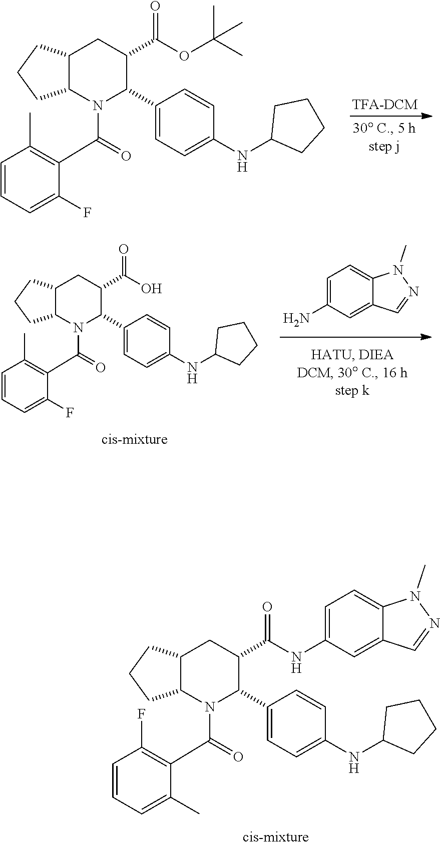

Step a) To a flask charged with finely cut sodium (27.33 g, 1.19 mol, 28.18 mL) in MTBE (1.5 L) at 0° C. was added dropwise of a solution of cyclopentanone (50 g, 594.41 mmol, 52.63 mL) and ethyl formate (46.23 g, 624.14 mmol, 50.20 mL) in MTBE (500 mL). The mixture was stirred at 10° C. for 16 h. The precipitate was filtered, washed with MTBE and dried to give the desired compound [(Z)-(2-oxocyclopentylidene)methoxy]sodium (22 g, 164.05 mmol, 27.60% yield) as a yellow solid. ¹H NMR (400 MHz, D₂O) δ 1.61 (2 H, quin, J=7.52 Hz) 2.09 (2H, t, J=7.83 Hz) 2.23 (2H, t, J=7.21 Hz) 8.24 (1 H, s) 8.54 (1 H, s).

Step b) To a mixture of (2-oxocyclopentylidene)methoxysodium (22 g, 164.05 mmol) in toluene (600 mL) was added 2-cyanoacetamide (30.34 g, 360.90 mmol). Then a solution make up of HOAc (1 M, 73.82 mL) and piperadine (1 M, 73.82 mL) in DCM (73 mL) were added. The mixture was stirred at 120° C. for 16 h. The reaction mixture was added to 500 mL H₂O and extracted with DCM (500 mL×2). The aqueous phase was acidified with 4 M HCl and was extracted with DCM (500 mL×2). The combined organic phase was dried with anhydrous Na₂SO₄, filtered and concentrated under vacuum to give target compound 2-oxo-1,5,6,7-tetrahydrocyclopenta[b]pyridine-3-carbonitrile (3 g, crude) as a yellow solid.

Step c) 2-oxo-1,5,6,7-tetrahydrocyclopenta[b]pyridine-3-carbonitrile (17 g, 106.14 mmol) was added to POCl₃ (99.00 g, 645.67 mmol, 60 mL). The mixture was stirred at 110° C. for 16 h. The most of POCl₃ was evaporated under vacuum to give crude product. The crude product was added to 50 mL of H₂O and 50 mL of DCM, then the mixture was stirred at 20° C. for 1 h. The solution was basified by saturated NaHCO₃ solution and extracted with DCM (400 mL×3). The combined organic phase was dried with anhydrous Na₂SO₄ and filtered through a pad of 100 g silica gel. The filtrate was evaporated under vacuum to give 2-chloro-6,7-dihydro-5H-cyclopenta[b]-pyridine-3-carbonitrile (16 g) as a pale yellow solid. ¹H NMR (400 MHz, CDCl₃) δ 2.16-2.25 (2H, m), 2.96 (2H, t, J=7.61 Hz), 3.06 (2H, t, J=7.83 Hz), 7.74 (1 H, s).

Step d) A solution of K₂CO₃ (36.03 g, 260.67 mmol) in H₂O (100 mL) was added to a mixture of 2-chloro-6,7-dihydro-5H-cyclopenta[b]pyridine-3-carbonitrile (16 g, 86.89 mmol), (4-nitrophenyl) boronic acid (18.86 g, 112.96 mmol) and Pd(PPh₃)₄ (10.04 g, 8.69 mmol) in dioxane (100 mL). The mixture was stirred at 100° C. under N₂ for 16 h. The result mixture was extracted with DCM (500 mL×2). The combined organic phase was washed with brine (500 mL), dried with anhydrous Na₂SO₄, filtered and evaporated under vacuum to give crude product. The crude product was triturated with EtOAc (50 mL) at 20° C. for 5 min. The suspension was filtered. The filter cake was washed with EtOAc 50 (mL) and dried under vacuum to give compound 2-(4-nitrophenyl)-6,7-dihydro-5H-cyclopenta[b]pyridine-3-carbonitrile (14 g, 52.78 mmol, 60.74% yield) as a pale yellow solid. ¹H NMR (400 MHz, CDCl₃) δ 2.27 (2 H, quin, J=7.65 Hz), 3.08 (2H, t, J=7.40 Hz), 3.17 (2H, t, J=7.78 Hz), 7.89 (1 H, s), 8.04-8.10 (2H, m), 8.33-8.41 (2H, m). LC-MS: (ES) m/z 266.1 (M+H⁺).

Step e) The 2-(4-nitrophenyl)-6,7-dihydro-5H-cyclopenta[b]pyridine-3-carbonitrile (10 g, 37.70 mmol) was added a solution of H₂SO₄ (110.92 g, 1.13 mol, 60.28 mL) in H₂O (60 mL). The mixture was stirred at 110° C. for 16 h. The reaction was cooled to 20° C., and was basified by 5 M NaOH to pH=5. Then the white solid was formed. The solid was filtered, washed with 150 mL H₂O and evaporated under vacuum to give 2-(4-nitrophenyl)-6,7-dihydro-5H-cyclopenta [b] pyridine-3-carboxylic acid (10 g, 35.18 mmol, 93.32% yield) as a white solid. ¹H NMR (400 MHz, DMSO-d₆) δ 2.09 (2 H, quin, J=7.52 Hz), 2.97 (4 H, br t, J=7.58 Hz), 7.70 (2H, d, J=8.56 Hz), 8.00 (1 H, s), 8.24 (2H, d, J=8.56 Hz), 13.13 (1 H, br s).

Step f) The tert-butoxycarbonyl tert-butyl carbonate (15.36 g, 70.36 mmol, 16.16 mL) was added to a mixture of 2-(4-nitrophenyl)-6,7-dihydro-5H-cyclopenta[b]pyridine-3-carboxylic acid (10 g, 35.18 mmol) and DMAP (4.30 g, 35.18 mmol) in THF (100 mL). The solution was stirred at 60° C. for 3 h. Another portion of tert-butoxycarbonyl tert-butyl carbonate (7.68 g, 35.18 mmol, 8.08 mL) and DMAP (2.15 g, 17.59 mmol) were added and the mixture was stirred at 60° C. for another 16 h. Another portion of tert-butoxycarbonyl tert-butyl carbonate (7.68 g, 35.18 mmol, 8.08 mL) and DMAP (2.15 g, 17.59 mmol) were added and stirred at 60° C. for 3 h. The reaction mixture was added to 100 mL H₂O and extracted with EtOAc (100 mL×2). The combined organic phase was washed with brine, dried with anhydrous Na₂SO₄, filtered and evaporated under vacuum to give a residue. The residue was purified by column chromatography (SiO₂, petroleum ether/Et-OAc=100:0 to 3:1) to give tert-butyl-2-(4-nitrophenyl)-6,7-dihydro-5H-cyclopenta[b]-pyridine-3-carboxylate (11.3 g, 33.20 mmol, 94.37% yield) as a pale yellow solid. ¹H NMR (400 MHz, CDCl₃) δ 1.29 (9 H, s), 2.20 (2 H, quin, J=7.64 Hz), 2.98-3.13 (4H, m), 7.63 (2H, d, J=8.56 Hz), 7.94 (1 H, s), 8.27 (2H, d, J=8.80 Hz). LC-MS: (ES) m/z 341.1 (M+H⁺).

Step g) PtO₂ (1.33 g, 5.88 mmol, 0.5 eq) was added to a solution of tert-butyl 2-(4-nitrophenyl)-6,7-dihydro-5H-cyclopenta[b]pyridine-3-carboxylate (4 g, 11.75 mmol) and HCl/dioxane (4 M, 5.88 mL) in MeOH (100 mL). The solution was stirred at 20° C. under H₂ (15 psi) for 2 h. The reaction mixture was filtered and the filtrate was evaporated under vacuum to give crude product. The crude product was added to 30 mL of H₂O and basified with saturate NaHCO₃ solution. The mixture was extracted with DCM (40 mL×2). The combined organic layers were washed with brine, dried over anhydrous Na₂SO₄, filtered and concentrated under vacuum to give a residue. The residue was purified by column chromatography (SiO₂, DCM:MeOH=100/0 to 100/1) to give compound cis-tert-butyl2-(4-aminophenyl)-2,3, 4,4a,5,6,7,7a-octahydro-1H-cyclopenta[b]pyridine-3-carboxylate (1.5 g, 4.65 mmol, 39.53% yield, 98% purity) as a yellow oil. ¹HNMR (400 MHz, CDCl₃) δ 1.09 (9 H, s), 1.52-1.81 (7H, m), 1.91-2.00 (2H, m), 2.01-2.08 (1H, m), 2.70 (1 H, q, J=5.95 Hz), 3.20 (1 H, td, J=6.54, 2.81 Hz), 3.48 (2 H, br s), 3.82 (1H, d, J=5.62 Hz), 6.55 (2H, d, J=8.31 Hz), 7.06 (2H, d, J=8.56 Hz). LC-MS: (ES) m/z 317.2 (M+H⁺).

Step h) To a mixture of cis-tert-butyl-2-(4-aminophenyl)-2,3,4,4a,5,6,7,7a-octahydro-1H-cyclo penta[b]pyridine-3-carboxylate (1.5 g, 4.74 mmol) and cyclopentanone (518.35 mg, 6.16 mmol, 545.64 μL) in MeOH (30 mL) was added HOAc (569.31 mg, 9.48 mmol, 542.20 μL) and NaBH₃CN (893.64 mg, 14.22 mmol) at 0° C. Then the mixture was stirred at 20° C. for 16 h. The reaction mixture was basified with NaHCO₃ solution and extracted with DCM (50 mL×2). The combined organic layers were washed with brine (40 mL), dried over anhydrous Na₂SO₄, filtered and concentrated under reduced pressure to give crude product. The crude product was purified by prep-HPLC (column: YMC-Triart Prep C18 150×40 mm×7 m; mobile phase: [water (0.1% TFA)-ACN]; B %: 20%-50%, 10 min). The pure fraction was basified with NaHCO₃ solution and extracted with DCM (500 mL×2). The combined organic layers were washed with brine (300 mL), dried over anhydrous Na₂SO₄, filtered and concentrated under reduced pressure to give compound cis-tert-butyl-2-[4-(cyclopentylamino)phenyl]-2,3,4,4a,5, 6,7,7a-octahydro-1H-cyclopenta[b]pyridine-3-carboxylate (1.2 g, 3.12 mmol, 65.83% yield, 100% purity) as a light yellow solid. ¹H NMR (400 MHz, CDCl₃) δ pm 1.09-1.22 (9H, m), 1.33 (2 H, br dd, J=8.80, 3.91 Hz), 1.39-1.79 (10H, m), 1.85-2.10 (7H, m), 2.11-2.25 (1H, m), 2.83 (1 H, q, J=5.54 Hz), 3.49 (1 H, br s), 3.66 (1 H, quin, J=6.11 Hz), 4.16 (1H, br d, J=5.14 Hz), 6.44 (2H, d, J=8.56 Hz), 7.14 (2 H, d, J=8.56 Hz). LC-MS: (ES) m/z 385.3 (M+H⁺).

Step i) To a solution of cis-tert-butyl 2-[4-(cyclopentylamino)phenyl]-2,3,4,4a,5,6,7,7a-octahydro-1H-cyclopenta[b]pyridine-3-carboxylate (800 mg, 2.08 mmol) and DIEA (537.73 mg, 4.16 mmol, 724.70 μL) in DCM (30 mL) was added dropwise of a solution of 2-fluoro-6-methyl-benzoyl chloride (359.03 mg, 2.08 mmol) in DCM (10 mL) at 0° C. The mixture was stirred at 0° C. for 1 h. The reaction mixture was added 20 mL of H₂O and was extracted with DCM (2×30 mL). The combined organic layers were washed with brine, dried over anhydrous Na₂SO₄, filtered and evaporated under vacuum to give a residue. The residue was purified by column chromatography (SiO₂, petroleum ether/EtOAc=100/0 to 3/1) to give compound cis-tert-butyl2-[4-(cyclopentylamino)phenyl]-1-(2-fluoro-6-methyl-benzoyl)-2,3,4,4a,5,6,7,7a-octahydrocyclopenta[b]pyridine-3-carboxylate (1 g, 1.92 mmol, 92.32% yield) as a white solid. ¹H NMR (400 MHz, CDCl₃) δ 0.81-0.97 (3H, m), 0.99-1.19 (2H, m), 1.28-1.38 (9H, m), 1.39-1.50 (4H, m), 1.58-1.76 (5H, m), 1.92-2.10 (6H, m), 2.28-2.40 (3H, m), 2.68-2.99 (1H, m), 3.43-3.80 (3H, m), 6.32-6.52 (2H, m), 6.57 (1H, dd, J=14.55, 5.26 Hz), 6.86-6.94 (1H, m), 6.96-7.01 (1H, m) 7.16-7.23 (1H, m) 7.30 (1H, d, J=8.31 Hz). LC-MS: (ES) m/z 521.3 (M+H⁺).

Step j) The cis-tert-butyl 2-[4-(cyclopentylamino)phenyl]-1-(2-fluoro-6-methyl-benzoyl)-2,3,4,4a,5,6,7,7a-octahydrocyclopenta[b]pyridine-3-carboxylate (1 g, 1.92 mmol) was dissolved in DCM (20 mL). Then TFA (6.48 g, 56.87 mmol, 4.21 mL) was added. The mixture was stirred at 15° C. for 16 h. The reaction mixture was evaporated under vacuum to remove most of solvent. Then 20 mL H₂O was added. Then the mixture was extracted with EtOAc (30 mL×2). The combined organic layers were washed with brine (30 mL), dried over anhydrous Na₂SO₄, filtered and the filtrate was concentrated under reduced pressure to give cis-2-[4-(cyclopentylamino)-phenyl]-1-(2-fluoro-6-methyl-benzoyl)-2,3,4,4a,5,6,7,7a-octahydrocyclopenta[b]pyridine-3-carboxylic acid (850 mg, 1.83 mmol, 95.27% yield) as a pale yellow solid. ¹H NMR (400 MHz, CDCl₃) δ 0.75-1.01 (1H, m), 1.13-1.31 (2H, m), 1.36-1.53 (2H, m), 1.58-2.06 (12H, m), 2.20 (1H, br d, J=12.30 Hz), 2.27-2.39 (3H, m), 2.93-3.25 (1H, m), 3.62-3.75 (1H, m), 3.77-3.96 (2H, m), 6.65-6.78 (1H, m), 6.92-7.11 (2H, m), 7.29-7.38 (2H, m), 7.46-7.62 (2H, m), 10.57 (1 H, br s). LC-MS: (ES) m z 465.3 (M+H⁺).

Step k) To a solution of cis-2-[4-(cyclopentylamino)phenyl]-1-(2-fluoro-6-methyl-benzoyl)-2,3,4,4a,5,6,7,7a-octahydrocyclopenta[b]pyridine-3-carboxylic acid (20 mg, 43.05 mol) in DCM (0.5 mL) was added HATU (20 mg, 52.60 mol) and DIEA (14.10 mg, 109.08 umol, 19 μL). The mixture was stirred at 30° C. for 0.5 h. Then the 1-methylindazol-5-amine (8.24 mg, 55.97 mol, 5.36 μL) was added and the mixture was stirred at 30° C. for another 15.5 hr. The reaction mixture was filtered and concentrated under reduced pressure to give a residue. The residue was purified by prep-HPLC (HCl condition, column: Agela DuraShell C18 150×25 mm×5 μm; mobile phase: [water (0.05% HCl)-ACN]; B %: 35%-65%, 8 min) to give 2-[4-(cyclopentylamino)phenyl]-1-(2-fluoro-6-methyl-benzoyl)-N-(1-me thylindazol-5-yl)-2,3,4,4a,5,6,7,7a-octahydrocyclopenta[b]-pyridine-3-carboxamide (13 mg, 19.60 mol, 45.52% yield, 95% purity, HCl) as a light yellow solid. ¹H NMR (400 MHz, METHANOL-d₄) δ 1.23-1.40 (m, 3H), 1.68 (br s, 2H), 1.83 (br s, 3H), 1.91-2.04 (m, 3H), 2.05-2.15 (m, 2H), 2.17-2.21 (m, 1 H), 2.42 (s, 2H), 3.19-3.27 (m, 1 H), 3.78-4.00 (m, 2H), 4.02-4.06 (m, 3H), 6.53-6.71 (m, 1H), 7.05 (br t, J=8.56 Hz, 1 H), 7.11-7.21 (m, 1H), 7.34-7.51 (m, 5H), 7.86-7.98 (m, 4H). LC-MS: (ES) m/z 594.3 (M+H⁺).

### Example S64: Synthesis of (2R,3S,4aR,7aR)-2-(4-(cyclopentylamino)phenyl)-1-(2-fluoro-6-methylbenzoyl)-N-(1-methyl-1H-indazol-5-yl)octahydro-1H-cyclopenta[b]pyridine-3-carboxamide (Compound No. 49)

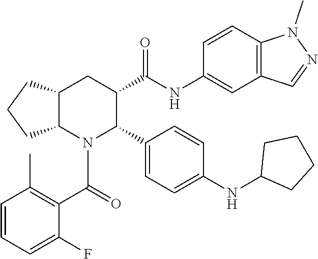 [SMILES: Cc1cccc(F)c1C(=O)N1[C@@H]2CCC[C@@H]2C[C@H](C(=O)Nc2ccc3c(cnn3C)c2)[C@@H]1c1ccc(NC2CCCC2)cc1]

The title compound was synthesized in similar fashion as Example S63. ¹H NMR (400 MHz, METHANOL-d₄) δ 1.20-1.47 (3H, m), 1.48-1.89 (10H, m), 1.90-2.05 (2H, m), 2.06-2.16 (2H, m), 2.17-2.35 (2H, m), 2.37-2.49 (2H, m), 3.19-3.29 (1H, m), 3.76-4.01 (2H, m), 4.02-4.10 (3H, m), 6.54-6.74 (1H, m), 7.01-7.11 (1H, m), 7.11-7.23 (1 H, m), 7.34-7.56 (5H, m), 7.81-8.01 (4H, m). LC-MS: (ES) m/z 594.3 (M+H⁺).

### Example S65: (2R,3S,4aR,7aR)-2-(4-(cyclopentylamino)phenyl)-1-(2-fluoro-6-methylbenzoyl)-N-(1-methyl-1H-indazol-6-yl)octahydro-1H-cyclopenta[b]pyridine-3-carboxamide (Compound No. 66)

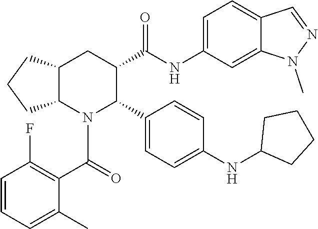 [SMILES: Cc1cccc(F)c1C(=O)N1[C@@H]2CCC[C@@H]2C[C@H](C(=O)Nc2ccc3cnn(C)c3c2)[C@@H]1c1ccc(NC2CCCC2)cc1]

The title compound was synthesized in similar fashion as Example S63. ¹H NMR (400 MHz, METHANOL-d₄) δ 1.23-1.47 (3H, m), 1.50-1.90 (10H, m), 1.90-2.04 (2H, m), 2.05-2.39 (4H, m), 2.44 (2 H, s), 3.22-3.31 (1H, m), 3.80-4.05 (5H, m), 6.58-6.77 (1H, m), 7.02-7.26 (3H, m), 7.37-7.42 (1H, m), 7.43-7.51 (2H, m), 7.63-7.72 (1H, m), 7.89-8.06 (4H, m). LC-MS: (ES) m/z 594.3 (M+H⁺).

### Example S66: (2R,3S,4aR,7aR)-2-(4-(cyclopentylamino)phenyl)-N-(4-(dimethylamino)phenyl)-1-(2-fluoro-6-methylbenzoyl)octahydro-1H-cyclopenta[b]pyridine-3-carboxamide (Compound No. 51)

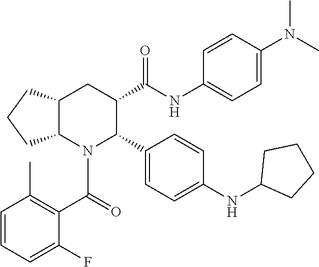 [SMILES: Cc1cccc(F)c1C(=O)N1[C@@H]2CCC[C@@H]2C[C@H](C(=O)Nc2ccc(N(C)C)cc2)[C@@H]1c1ccc(NC2CCCC2)cc1]

The title compound was synthesized in similar fashion as Example S63. ¹H NMR (400 MHz, METHANOL-d₄) δ 1.15-1.40 (m, 4H), 1.48-1.58 (m, 1H), 1.66-1.77 (m, 5 H), 1.85 (br s, 2H), 1.94-2.12 (m, 4H), 2.17-2.31 (m, 2H), 2.41 (s, 2H), 3.19-3.27 (m, 7 H), 3.74-4.01 (m, 2H), 6.55-6.70 (m, 1H), 7.02-7.11 (m, 1H), 7.11-7.21 (m, 1H), 7.34-7.45 (m, 3H), 7.53-7.61 (m, 2H), 7.67-7.81 (m, 2H), 7.84-7.92 (m, 2H). LC-MS: (ES) m z 583.4 (M+H⁺).

### Example S67: Synthesis of (2R,3S,4aR,7aR)-2-(4-(cyclopentylamino)phenyl)-1-(2-fluoro-6-methylbenzoyl)-N-(tetrahydro-2H-pyran-4-yl)octahydro-1H-cyclopenta[b]pyridine-3-carboxamide (Compound No. 52)

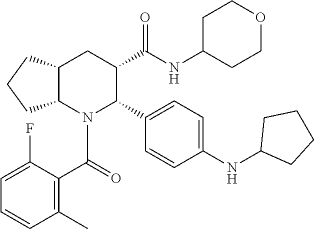 [SMILES: Cc1cccc(F)c1C(=O)N1[C@@H]2CCC[C@@H]2C[C@H](C(=O)NC2CCOCC2)[C@@H]1c1ccc(NC2CCCC2)cc1]

The title compound was synthesized in similar fashion as Example S63. ¹H NMR (400 MHz, METHANOL-d₄) δ 1.10-1.62 (m, 7H), 1.64-1.91 (m, 9H), 1.94-2.09 (m, 4 H), 2.10-2.25 (m, 2H), 2.36 (s, 2H), 2.92-3.04 (m, 1H), 3.34-3.51 (m, 2H), 3.70-4.05 (m, 5H), 6.44-6.60 (m, 1H), 6.90-7.07 (m, 1H), 7.09-7.19 (m, 1H), 7.23-7.37 (m, 1 H), 7.38-7.49 (m, 2H), 7.78-7.92 (m, 2H). LC-MS: (ES) m/z 548.3 (M+H⁺).

### Example S68: Synthesis of (2R,3S,4aR,7aR)-2-(4-(cyclopentylamino)phenyl)-1-(2-fluoro-6-methylbenzoyl)-N-(1-methylpiperidin-4-yl)octahydro-1H-cyclopenta[b]pyridine-3-carboxamide (Compound No. 53)

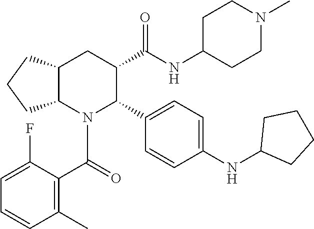 [SMILES: Cc1cccc(F)c1C(=O)N1[C@@H]2CCC[C@@H]2C[C@H](C(=O)NC2CCN(C)CC2)[C@@H]1c1ccc(NC2CCCC2)cc1]

The title compound was synthesized in similar fashion as Example S63. ¹H NMR (400 MHz, METHANOL-d₄) δ 1.04-1.37 (m, 3H), 1.39-1.60 (m, 2H), 1.61-1.81 (m, 7 H), 1.87 (br d, J=9.03 Hz, 3H), 1.95-2.08 (m, 5H), 2.14-2.25 (m, 2H), 2.32-2.44 (m, 2 H), 2.76-2.90 (m, 3H), 2.93-3.17 (m, 3H), 3.32-3.65 (m, 3H), 3.70-4.11 (m, 3H), 6.48-6.63 (m, 1H), 6.96-7.08 (m, 1H), 7.10-7.20 (m, 1H), 7.28-7.42 (m, 1H), 7.44-7.54 (m, 2H), 7.75-7.91 (m, 2H). LC-MS: (ES) m/z 561.3 (M+H⁺).

### Example S69: Synthesis of (2R,3S,4aR,7aR)-2-(4-(cyclopentylamino)phenyl)-1-(2-fluoro-6-methylbenzoyl)-N-(1-methyl-1H-pyrazol-4-yl)octahydro-1H-cyclopenta[b]pyridine-3-carboxamide (Compound No. 54)

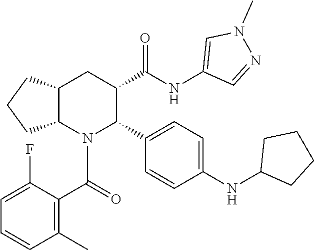 [SMILES: Cc1cccc(F)c1C(=O)N1[C@@H]2CCC[C@@H]2C[C@H](C(=O)Nc2cnn(C)c2)[C@@H]1c1ccc(NC2CCCC2)cc1]

The title compound was synthesized in similar fashion as Example S63. LC-MS: (ES) m/z 544.3 (M+H⁺).

### Example S70: (2R,3S,4aR,7aR)-2-(4-(cyclopentylamino)phenyl)-1-(2-fluoro-6-methylbenzoyl)-N-(4-methyl-3-(trifluoromethyl)benzyl)octahydro-1H-cyclopenta[b]pyridine-3-carboxamide (Compound No. 185)

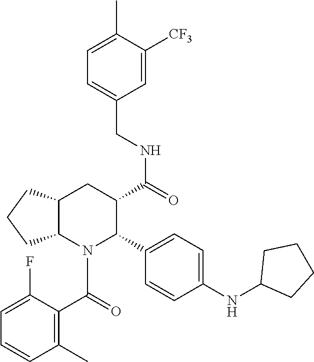 [SMILES: Cc1ccc(CNC(=O)[C@H]2C[C@H]3CCC[C@H]3N(C(=O)c3c(C)cccc3F)[C@H]2c2ccc(NC3CCCC3)cc2)cc1C(F)(F)F]

The title compound was synthesized in similar fashion as Example S64. ¹H NMR (400 MHz, METHANOL-d₄) δ 0.98-1.34 (m, 3H), 1.40-1.48 (m, 1H), 1.71 (br s, 6H), 1.87 (br s, 2H), 1.93-2.11 (m, 4H), 2.17 (td, J=12.80, 6.78 Hz, 1H), 2.27 (s, 1H), 2.37 (s, 2H), 2.44 (br s, 3H), 3.04 (br dd, J=9.66, 4.89 Hz, 1H), 3.70-4.02 (m, 2H), 4.24-4.52 (m, 2H), 6.57-6.73 (m, 1H), 7.05 (t, J=8.91 Hz, 1H), 7.11-7.19 (m, 1H), 7.23-7.42 (m, 5 H), 7.44-7.55 (m, 1H), 7.67-7.84 (m, 2H), 8.77-8.96 (m, 1H). LC-MS: (ES) m/z 636.4 (M+H⁺).

### Example S71: cis-2-(4-(cyclopentylamino)phenyl)-1-(2-fluoro-6-methylbenzoyl)-N-(3-(trifluoro-methyl)phenyl)octahydro-1H-cyclopenta[b]pyridine-3-carboxamide (Compound No. 46)

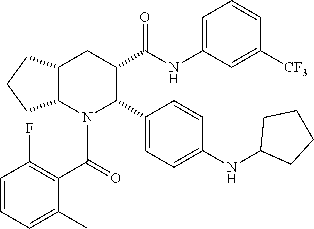

The title compound was synthesized in similar fashion as Example S63. ¹H NMR (400 MHz, METHANOL-d₄) δ 1.26-1.35 (m, 3H), 1.66 (br s, 2H), 1.74-1.85 (m, 3H), 1.98 (br t, J=16.14 Hz, 2H), 2.06-2.14 (m, 1H), 2.17-2.27 (m, 2H), 2.41 (s, 2H), 3.19 (dt, J=10.52, 5.26 Hz, 1H), 3.75-3.97 (m, 2H), 6.53-6.67 (m, 1H), 7.05 (td, J=8.68, 3.91 Hz, 1H), 7.10-7.22 (m, 2H), 7.25 (br d, J=8.56 Hz, 1H), 7.34-7.41 (m, 2H), 7.42-7.51 (m, 1 H), 7.56-7.72 (m, 1H), 7.79 (d, J=8.56 Hz, 2H), 7.89-8.00 (m, 1H), 10.28 (br d, J=4.65 Hz, 1H). LC-MS: (ES) m/z 608.3 (M+H⁺).

### Example S72: Synthesis of cis-N-(3-chlorophenyl)-2-(4-(cyclopentylamino)phenyl)-1-(2-fluoro-6-methylbenzoyl)octahydro-1H-cyclopenta[b]pyridine-3-carboxamide (Compound No. 55)

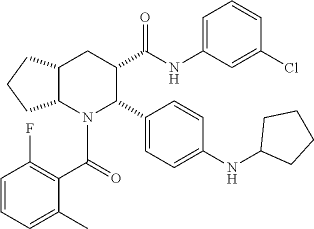

The title compound was synthesized in similar fashion as Example S63. ¹H NMR (400 MHz, METHANOL-d₄) δ 1.12-1.36 (3H, m) 1.37-1.80 (10H, m) 1.86-2.22 (5H, m) 2.23-2.42 (3H, m) 2.96-3.10 (1H, m) 3.65-3.89 (2H, m) 6.50-6.58 (2H, m) 6.97-7.16 (3H, m) 7.17-7.24 (1H, m) 7.25-7.45 (4H, m) 7.56-7.68 (1H, m). LC-MS: (ES) m z 574.2 (M+H⁺).

### Example S73: Synthesis of cis-2-(4-(cyclopentylamino)phenyl)-1-(2-fluoro-6-methylbenzoyl)-N-(3-fluorophenyl)octahydro-1H-cyclopenta[b]pyridine-3-carboxamide (Compound No. 56)

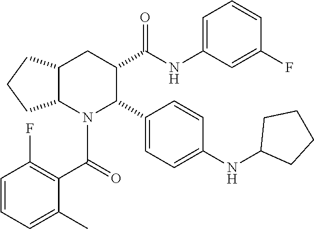

The title compound was synthesized in similar fashion as Example S63. ¹H NMR (400 MHz, METHANOL-d₄) δ 1.19-1.48 (m, 3H), 1.56-1.77 (m, 6H), 1.78-1.91 (m, 2 H), 1.92-2.11 (m, 4H), 2.14-2.29 (m, 2H), 2.33-2.43 (m, 2H), 3.03-3.25 (m, 1H), 3.72-4.03 (m, 2H), 6.49-6.76 (m, 1H), 6.77-6.98 (m, 1H), 7.02-7.31 (m, 4H), 7.33-7.54 (m, 4H), 7.66-7.87 (m, 2H). LC-MS: (ES) m/z 558.4 (M+H⁺).

### Example S74: Synthesis of cis-2-(4-(cyclopentylamino)phenyl)-1-(2-fluoro-6-methylbenzoyl)-N-(pyridin-3-yl)octahydro-1H-cyclopenta[b]pyridine-3-carboxamide (Compound No. 57)

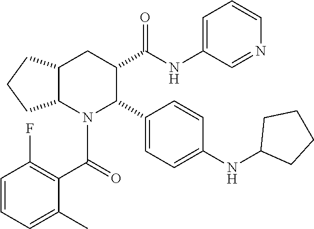

The title compound was synthesized in similar fashion as Example S63. ¹H NMR (400 MHz, METHANOL-d₄) δ 1.44-1.52 (m, 4H), 1.56-1.64 (m, 4H), 1.71 (br d, J=5.62 Hz, 4H), 1.94-2.07 (m, 4H), 2.13-2.22 (m, 2H), 2.27 (s, 1H), 2.39 (s, 2H), 3.05-3.17 (m, 1H), 3.69-3.78 (m, 2H), 6.53-6.64 (m, 4H), 7.01-7.08 (m, 1H), 7.14 (dd, J=11.00, 7.83 Hz, 1H), 7.33-7.41 (m, 3H), 7.45 (d, J=8.56 Hz, 1H), 7.92-8.05 (m, 1H), 8.23 (ddd, J=8.93, 4.77, 1.22 Hz, 1H), 8.63-8.72 (m, 1H). LC-MS: (ES) m/z 541.3 (M+H⁺).

### Example S75: Synthesis of cis-2-(4-(cyclopentylamino)phenyl)-1-(2-fluoro-6-methylbenzoyl)-N-(2-methylpyrimidin-5-yl)octahydro-1H-cyclopenta[b]pyridine-3-carboxamide (Compound No. 58)

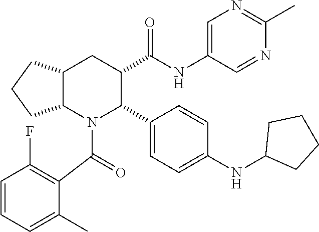 [SMILES: Cc1ncc(NC(=O)[C@H]2C[C@H]3CCC[C@H]3N(C(=O)c3c(C)cccc3F)[C@H]2c2ccc(NC3CCCC3)cc2)cn1]

The title compound was synthesized in similar fashion as Example S63. ¹H NMR (400 MHz, METHANOL-d₄) δ 1.16-1.42 (m, 3H), 1.71 (br s, 6H), 1.85 (br s, 3H), 1.95-2.04 (m, 3H), 2.12-2.28 (m, 3H), 2.39-2.42 (m, 2H), 2.72-2.82 (m, 3H), 3.12-3.30 (m, 1H), 3.82-4.08 (m, 2H), 6.64-6.78 (m, 1H), 7.04-7.22 (m, 2H), 7.34-7.52 (m, 3H), 7.82-7.94 (m, 2H), 9.05-9.29 (m, 2H). LC-MS: (ES) m/z 556.4 (M+H⁺).

### Example S76: Synthesis of cis-2-(4-(cyclopentylamino)phenyl)-1-(2-fluoro-6-methylbenzoyl)-N-(1-(oxetan-3-yl)-1H-indazol-6-yl)octahydro-1H-cyclopenta[b]pyridine-3-carboxamide (Compound No. 59)

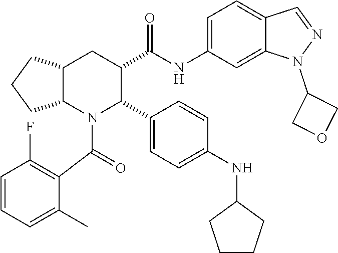 [SMILES: Cc1cccc(F)c1C(=O)N1[C@@H]2CCC[C@@H]2C[C@H](C(=O)Nc2ccc3cnn(C4COC4)c3c2)[C@@H]1c1ccc(NC2CCCC2)cc1]

The title compound was synthesized in similar fashion as Example S63. ¹H NMR (400 MHz, METHANOL-d₄) δ 1.14-1.52 (4H, m), 1.61-1.92 (9H, m), 1.92-2.27 (6H, m), 2.28-2.54 (3H, m), 3.11-3.31 (1H, m), 3.78-4.16 (4H, m), 5.09-5.29 (1H, m), 6.57-6.78 (1H, m), 7.03-7.25 (3H, m), 7.36-7.54 (3H, m), 7.61-7.78 (2H, m), 7.89-8.13 (3 H, m). LC-MS: (ES) m/z 636.3 (M+H⁺).

### Example S77: Synthesis of cis-N-(4-chloro-3-(trifluoromethyl)phenyl)-2-(4-(cyclopentylamino)-phenyl)-1-(2-fluoro-6-methylbenzoyl)octahydro-1H-cyclopenta[b]pyridine-3-carboxamide (Compound No. 60)

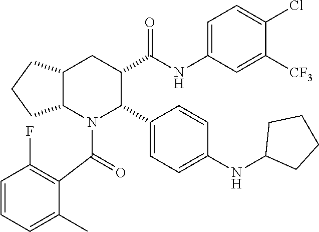 [SMILES: Cc1cccc(F)c1C(=O)N1[C@@H]2CCC[C@@H]2C[C@H](C(=O)Nc2ccc(Cl)c(C(F)(F)F)c2)[C@@H]1c1ccc(NC2CCCC2)cc1]

The title compound was synthesized in similar fashion as Example S63. ¹H NMR (400 MHz, METHANOL-d₄) δ 1.35 (br s, 3H), 1.56-1.71 (m, 6H), 1.79 (br d, J=5.62 Hz, 3 H), 1.93-2.11 (m, 4H), 2.21 (s, 1H), 2.40 (s, 2H), 3.15 (br dd, J=10.15, 5.50 Hz, 1H), 3.75-3.94 (m, 2H), 6.52-6.67 (m, 1H), 7.05 (br t, J=8.56 Hz, 2H), 7.15 (br dd, J=17.00, 7.46 Hz, 2H), 7.33-7.42 (m, 1H), 7.47-7.57 (m, 1H), 7.63-7.76 (m, 3H), 8.00-8.10 (m, 1 H). LC-MS: (ES) m/z 642.3 (M+H⁺).

### Example S78: Synthesis of cis-2-(4-(cyclopentylamino)phenyl)-N-(4-fluoro-3-(trifluoromethyl)-phenyl)-1-(2-fluoro-6-methylbenzoyl)octahydro-1H-cyclopenta[b]pyridine-3-carboxamide (Compound No. 61)

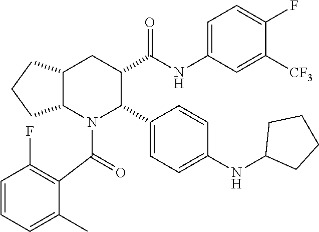 [SMILES: Cc1cccc(F)c1C(=O)N1[C@@H]2CCC[C@@H]2C[C@H](C(=O)Nc2ccc(F)c(C(F)(F)F)c2)[C@@H]1c1ccc(NC2CCCC2)cc1]

The title compound was synthesized in similar fashion as Example S63. ¹H NMR (400 MHz, METHANOL-d₄) δ 1.19-1.39 (m, 3H), 1.54-1.76 (m, 6H), 1.83 (br s, 3H), 1.91-2.04 (m, 2H), 2.04-2.15 (m, 2H), 2.16-2.30 (m, 2H), 2.40 (s, 2H), 3.15-3.25 (m, 1H), 3.76-4.01 (m, 2H), 6.53-6.69 (m, 1H), 7.02-7.10 (m, 1H), 7.11-7.20 (m, 1H), 7.27 (q, J=9.70 Hz, 1H), 7.33-7.43 (m, 3H), 7.62-7.77 (m, 1H), 7.81-7.90 (m, 2H), 7.94-8.08 (m, 1H), 10.32 (s, 1H). LC-MS: (ES) m/z 626.3 (M+H⁺).

### Example S79: Synthesis of cis-N-(3-cyano-4-methylphenyl)-2-(4-(cyclopentylamino)phenyl)-1-(2-fluoro-6-methylbenzoyl)octahydro-1H-cyclopenta[b]pyridine-3-carboxamide (Compound No. 62)

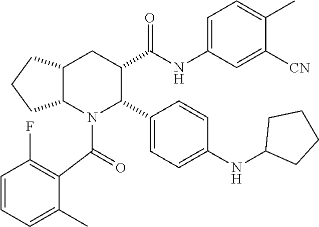 [SMILES: Cc1ccc(NC(=O)[C@H]2C[C@H]3CCC[C@H]3N(C(=O)c3c(C)cccc3F)[C@H]2c2ccc(NC3CCCC3)cc2)cc1C#N]

The title compound was synthesized in similar fashion as Example S63. ¹H NMR (400 MHz, METHANOL-d₄) δ 1.30-1.69 (8H, m), 1.74-1.98 (5H, m), 1.99-2.13 (2H, m), 2.16 (1H, s), 2.29 (1H, s), 2.30-2.38 (3H, m), 2.96 (1 H, br s), 3.57-3.82 (2H, m), 4.53 (3 H, br s), 6.39-6.52 (2H, m), 6.90-6.98 (1H, m), 6.99-7.07 (1H, m), 7.10-7.30 (3 H, m), 7.34 (1H, d, J=8.56 Hz), 7.42-7.53 (1H, m), 7.70-7.81 (1H, m). LC-MS: (ES) m z 579.3 (M+H⁺).

### Example S80: Synthesis of cis-2-(4-(cyclopentylamino)phenyl)-1-(2-fluoro-6-methylbenzoyl)-N-(6-methylpyridin-3-yl)octahydro-1H-cyclopenta[b]pyridine-3-carboxamide (Compound No. 63)

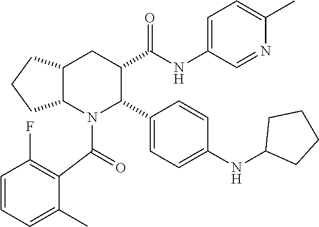 [SMILES: Cc1ccc(NC(=O)[C@H]2C[C@H]3CCC[C@H]3N(C(=O)c3c(C)cccc3F)[C@H]2c2ccc(NC3CCCC3)cc2)cn1]

The title compound was synthesized in similar fashion as Example S63. ¹H NMR (400 MHz, METHANOL-d₄) δ 1.05-1.36 (m, 3H), 1.47-1.57 (m, 1H), 1.63-1.92 (m, 8 H), 1.99 (br s, 2H), 2.07-2.21 (m, 2H), 2.22-2.31 (m, 2H), 2.41 (s, 2H), 2.69-2.76 (m, 3 H), 3.25-3.30 (m, 1H), 3.76-4.04 (m, 2H), 6.67-6.81 (m, 1H), 7.03-7.11 (m, 1H), 7.13-7.22 (m, 1H), 7.33-7.44 (m, 1H), 7.45-7.53 (m, 2H), 7.84-7.96 (m, 3H), 8.37-8.51 (m, 1H), 9.14-9.29 (m, 1H). LC-MS: (ES) m/z 555.3 (M+H⁺).

### Example S81: Synthesis of cis-2-(4-(cyclopentylamino)phenyl)-N-(3,4-dichlorophenyl)-1-(2-fluoro-6-methylbenzoyl)octahydro-1H-cyclopenta[b]pyridine-3-carboxamide (Compound No. 64)

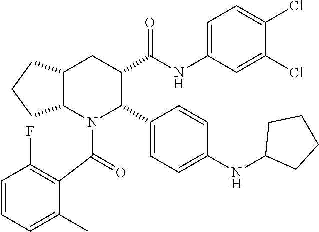 [SMILES: Cc1cccc(F)c1C(=O)N1[C@@H]2CCC[C@@H]2C[C@H](C(=O)Nc2ccc(Cl)c(Cl)c2)[C@@H]1c1ccc(NC2CCCC2)cc1]

The title compound was synthesized in similar fashion as Example S63. ¹H NMR (400 MHz, METHANOL-d₄) δ 1.28-1.33 (m, 3H), 1.53-1.71 (m, 6H), 1.72-1.85 (m, 3 H), 1.92-2.10 (m, 4H), 2.17-2.25 (m, 2H), 2.39 (s, 2H), 3.07-3.19 (m, 1H), 3.72-3.96 (m, 2H), 6.48-6.68 (m, 1H), 6.96-7.20 (m, 4H), 7.29-7.46 (m, 3H), 7.67 (br d, J=8.31 Hz, 2H), 7.77-7.89 (m, 1H). LC-MS: (ES) m/z 608.3 (M+H⁺).

### Example S82: Synthesis of cis-2-(4-(cyclopentylamino)phenyl)-N-(3,4-difluorophenyl)-1-(2-fluoro-6-methyl benzoyl)octahydro-1H-cyclopenta[b]pyridine-3-carboxamide (Compound No. 65)

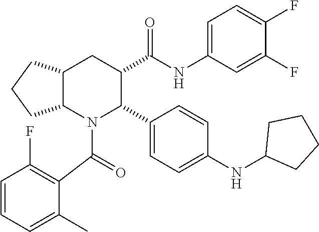 [SMILES: Cc1cccc(F)c1C(=O)N1[C@@H]2CCC[C@@H]2C[C@H](C(=O)Nc2ccc(F)c(F)c2)[C@@H]1c1ccc(NC2CCCC2)cc1]

The title compound was synthesized in similar fashion as Example S63. ¹H NMR (400 MHz, METHANOL-d₄) δ 1.18-1.58 (m, 3H), 1.64-1.78 (m, 6H), 1.84 (br s, 3H), 1.93-2.10 (m, 4H), 2.14-2.27 (m, 2H), 2.38-2.43 (m, 2H), 3.11-3.24 (m, 1H), 3.76-4.02 (m, 2H), 6.48-6.68 (m, 1H), 7.04-7.24 (m, 4H), 7.34-7.47 (m, 3H), 7.50-7.66 (m, 1H), 7.67-7.91 (m, 2H). LC-MS: (ES) m/z 576.3 (M+H⁺).

### Example S83: Synthesis of cis-2-(4-(cyclopentylamino)phenyl)-1-(2-fluoro-6-methylbenzoyl)-N-(1-methyl-1H-indazol-6-yl)octahydro-1H-cyclopenta[b]pyridine-3-carboxamide (Compound No. 66)

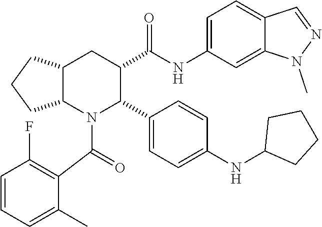 [SMILES: Cc1cccc(F)c1C(=O)N1[C@@H]2CCC[C@@H]2C[C@H](C(=O)Nc2ccc3cnn(C)c3c2)[C@@H]1c1ccc(NC2CCCC2)cc1]

The title compound was synthesized in similar fashion as Example S63. ¹H NMR (400 MHz, METHANOL-d₄) δ 1.18-1.41 (m, 3H), 1.55-1.75 (m, 6H), 1.82 (br d, J=6.11 Hz, 3H), 1.89-2.02 (m, 2H), 2.07-2.16 (m, 2H), 2.17-2.35 (m, 2H), 2.42 (s, 2H), 3.21-3.29 (m, 1H), 3.77-3.96 (m, 2H), 3.97-4.03 (m, 3H), 6.51-6.76 (m, 1H), 6.99-7.23 (m, 3H), 7.32-7.49 (m, 3H), 7.62-7.73 (m, 1H), 7.85-8.03 (m, 4H). LC-MS: (ES) m/z 594.4 (M+H⁺).

### Example S84: Synthesis of cis-N-(benzo[d]oxazol-6-yl)-2-(4-(cyclopentylamino)phenyl)-1-(2-fluoro-6-methylbenzoyl)octahydro-1H-cyclopenta[b]pyridine-3-carboxamide (Compound No. 67)

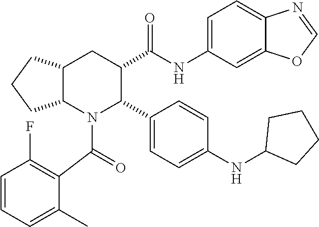 [SMILES: Cc1cccc(F)c1C(=O)N1[C@@H]2CCC[C@@H]2C[C@H](C(=O)Nc2ccc3ncoc3c2)[C@@H]1c1ccc(NC2CCCC2)cc1]

The title compound was synthesized in similar fashion as Example S63. ¹H NMR (400 MHz, METHANOL-d₄) δ 1.16-1.35 (m, 2H), 1.37-1.52 (m, 4H), 1.53-1.64 (m, 3 H), 1.70 (br d, J=5.87 Hz, 3H), 1.89-2.08 (m, 3H), 2.12-2.24 (m, 2H), 2.27 (s, 1H), 2.37-2.45 (m, 2H), 3.04-3.18 (m, 1H), 3.65-3.93 (m, 2H), 6.49-6.68 (m, 3H), 7.01-7.22 (m, 2H), 7.29-7.51 (m, 4H), 7.55-7.73 (m, 1H), 7.93-8.13 (m, 1H), 8.31-8.42 (m, 1 H). LC-MS: (ES) m/z 581.4 (M+H⁺).

### Example S85: Synthesis of cis-2-(4-(cyclopentylamino)phenyl)-1-(2-fluoro-6-methylbenzoyl)-N-(4-formami do-3-hydroxyphenyl)octahydro-1H-cyclopenta[b]pyridine-3-carboxamide (Compound No. 68)

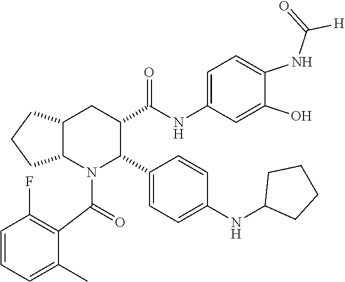 [SMILES: Cc1cccc(F)c1C(=O)N1[C@@H]2CCC[C@@H]2C[C@H](C(=O)Nc2ccc(NC=O)c(O)c2)[C@@H]1c1ccc(NC2CCCC2)cc1]

The title compound was synthesized in similar fashion as Example S63. ¹H NMR (400 MHz, METHANOL-d₄) δ 1.22-1.38 (m, 2H), 1.50-1.76 (m, 7H), 1.78-1.90 (m, 3 H), 1.92-2.13 (m, 4H), 2.15-2.31 (m, 2H), 2.37-2.47 (m, 2H), 3.11-3.22 (m, 1H), 3.76-4.01 (m, 2H), 6.49-6.66 (m, 1H), 6.77-6.95 (m, 1H), 7.02-7.09 (m, 1H), 7.10-7.25 (m, 2H), 7.32-7.46 (m, 3H), 7.71-7.93 (m, 3H), 8.19-8.26 (m, 1H), 9.93-9.99 (m, 1 H). LC-MS: (ES) m/z 599.3 (M+H⁺).

### Example S88: Synthesis of cis-N-(benzo[d]thiazol-6-yl)-2-(4-(cyclopentylamino)phenyl)-1-(2-fluoro-6-methylbenzoyl)octahydro-1H-cyclopenta[b]pyridine-3-carboxamide (Compound No. 69)

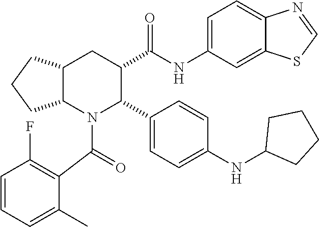 [SMILES: Cc1cccc(F)c1C(=O)N1[C@@H]2CCC[C@@H]2C[C@H](C(=O)Nc2ccc3ncsc3c2)[C@@H]1c1ccc(NC2CCCC2)cc1]

The title compound was synthesized in similar fashion as Example S63. ¹H NMR (400 MHz, METHANOL-d₄) δ 1.27-1.40 (m, 3H), 1.58-1.74 (m, 6H), 1.82 (br s, 3H), 1.96 (br s, 2H), 2.07-2.14 (m, 2H), 2.17 (s, 1H), 2.20-2.37 (m, 1H), 2.42 (s, 2H), 3.19-3.28 (m, 1H), 3.77-4.01 (m, 2H), 6.55-6.70 (m, 1H), 6.91-7.22 (m, 3H), 7.32-7.59 (m, 4H), 7.85-8.01 (m, 3H), 8.28-8.46 (m, 1H), 9.10-9.21 (m, 1H). LC-MS: (ES) m/z 597.3 (M+H⁺).

### Example S87: Synthesis of cis-2-(4-(cyclopentylamino)phenyl)-1-(2-fluoro-6-methylbenzoyl)-N-(3-(methyls ulfonyl)phenyl)octahydro-1H-cyclopenta[b]pyridine-3-carboxamide (Compound No. 70)

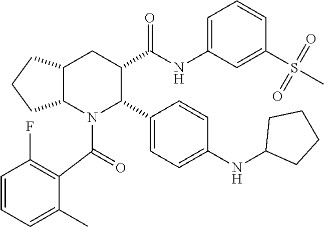 [SMILES: Cc1cccc(F)c1C(=O)N1[C@@H]2CCC[C@@H]2C[C@H](C(=O)Nc2cccc(S(C)(=O)=O)c2)[C@@H]1c1ccc(NC2CCCC2)cc1]

The title compound was synthesized in similar fashion as Example S63. ¹H NMR (400 MHz, METHANOL-d₄) δ 1.21-1.38 (m, 2H), 1.63-1.79 (m, 7H), 1.81-1.88 (m, 2 H), 1.92-2.12 (m, 5H), 2.14-2.31 (m, 2H), 2.37-2.44 (m, 2H), 3.05-3.16 (m, 3H), 3.19-3.28 (m, 1H), 3.75-4.02 (m, 2H), 5.94-6.14 (m, 0.3 H), 6.52-6.68 (m, 0.7 H), 7.04-7.20 (m, 2H), 7.36-7.49 (m, 3H), 7.53-7.71 (m, 3H), 7.75-7.92 (m, 2H), 8.11-8.29 (m, 1H), 10.31-10.47 (m, 1H). LC-MS: (ES) m/z 618.2 (M+H⁺).

### Example S88: Synthesis of cis-2-(4-(cyclopentylamino)phenyl)-N-(2,3-dihydrobenzo[b][1,4]-dioxin-6-yl)-1-(2-fluoro-6-methylbenzoyl)octahydro-1H-cyclopenta[b]pyridine-3-carboxamide (Compound No. 71)

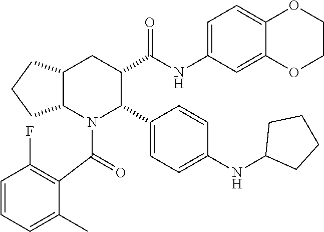 [SMILES: Cc1cccc(F)c1C(=O)N1[C@@H]2CCC[C@@H]2C[C@H](C(=O)Nc2ccc3c(c2)OCCO3)[C@@H]1c1ccc(NC2CCCC2)cc1]

The title compound was synthesized in similar fashion as Example S63. ¹H NMR (400 MHz, METHANOL-d₄) δ 1.20-1.40 (m, 2H), 1.42-1.62 (m, 2H), 1.62-1.92 (m, 8 H), 1.93-2.10 (m, 4H), 2.17 (s, 1H), 2.19-2.48 (m, 3H), 3.04-3.20 (m, 1H), 3.72-3.87 (m, 1H), 3.93-4.02 (m, 1H), 4.09-4.27 (m, 4H), 6.46-6.64 (m, 1H), 6.68-6.75 (m, 1 H), 6.80-6.96 (m, 1H), 7.01-7.05 (m, 1H), 7.05-7.27 (m, 2H), 7.31-7.45 (m, 2H), 7.79-7.91 (m, 2H). LC-MS: (ES) m/z 598.4 (M+H⁺).

### Example S89: Synthesis of cis-3-(6-chloro-1,2,3,4-tetrahydroisoquinoline-2-carbonyl)-2-(4-(cyclopentyl amino)phenyl)octahydro-1H-cyclopenta[b]pyridin-1-yl)(2-fluoro-6-methylphenyl)-methanone (Compound No. 72)

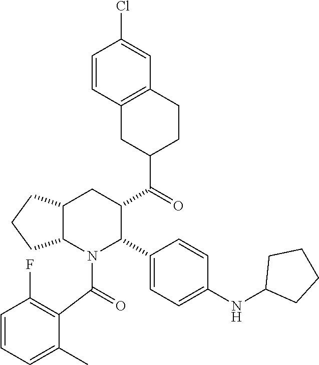 [SMILES: Cc1cccc(F)c1C(=O)N1[C@@H]2CCC[C@@H]2C[C@H](C(=O)C2CCc3cc(Cl)ccc3C2)[C@@H]1c1ccc(NC2CCCC2)cc1]

The title compound was synthesized in similar fashion as Example S63. ¹H NMR (400 MHz, METHANOL-d₄) δ 1.18-1.59 (m, 3H), 1.70 (br d, J=4.16 Hz, 6H), 1.79-1.91 (m, 3H), 1.92-2.05 (m, 3H), 2.11-2.33 (m, 2H), 2.38-2.55 (m, 2H), 2.94-3.19 (m, 1 H), 3.43-3.83 (m, 2H), 3.85-4.12 (m, 3H), 4.54-4.86 (m, 3H), 4.94-5.07 (m, 1H), 6.32-6.59 (m, 1H), 6.60-6.85 (m, 1H), 6.95-7.45 (m, 8H), 7.56-7.91 (m, 1H). LC-MS: (ES) m z 614.3 (M+H⁺).

### Example S90: Synthesis of cis-2-(4-(cyclopentylamino)phenyl)-1-(2-fluoro-6-methylbenzoyl)-N-(quinolin-7-yl)octahydro-1H-cyclopenta[b]pyridine-3-carboxamide (Compound No. 73)

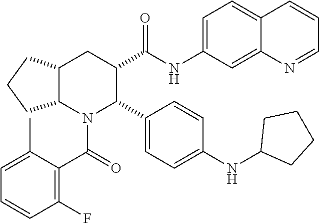

The title compound was synthesized in similar fashion as Example S63. ¹H NMR (400 MHz, METHANOL-d₄) δ 1.13-1.48 (3H, m), 1.49-1.87 (10H, m), 1.89-2.10 (3H, m), 2.12-2.29 (3H, m), 2.30-2.53 (3H, m), 3.34-3.41 (1H, m), 3.78-4.07 (2H, m), 6.66-6.84 (1H, m), 7.03-7.24 (2H, m), 7.26-7.54 (3H, m), 7.82-7.98 (4H, m), 8.16-8.31 (1 H, m), 8.81-9.09 (3H, m). LC-MS: (ES) m/z 591.5 (M+H⁺).

### Example S91: Synthesis of cis-2-(4-(cyclopentylamino)phenyl)-1-(2-fluoro-6-methylbenzoyl)-N-(1-methyl-1H-benzo[d]imidazol-6-yl)octahydro-1H-cyclopenta[b]pyridine-3-carboxamide (Compound No. 74)

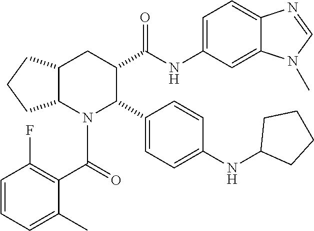

The title compound was synthesized in similar fashion as Example S63. ¹H NMR (400 MHz, METHANOL-d₄) δ 1.18-1.45 (4H, m), 1.47-1.89 (10H, m), 1.99 (2 H, br s), 2.09-2.26 (2H, m), 2.26-2.50 (3H, m), 3.12-3.29 (1H, m), 3.74-3.96 (2H, m), 3.97-4.15 (3H, m), 6.62-6.78 (1H, m), 7.03-7.24 (4H, m), 7.34-7.45 (1H, m), 7.57-7.70 (1 H, m), 7.72-7.88 (3H, m), 8.27-8.43 (1H, m), 9.25 (1 H, br s). LC-MS: (ES) m/z 594.3 (M+H⁺).

### Example S92: Synthesis of cis-N-(5-chloro-6-(2H-1,2,3-triazol-2-yl)pyridin-3-yl)-2-(4-(cyclo-pentylamino)phenyl)-1-(2-fluoro-6-methylbenzoyl)octahydro-1H-cyclopenta[b]pyridine-3-carboxamide (Compound No. 75)

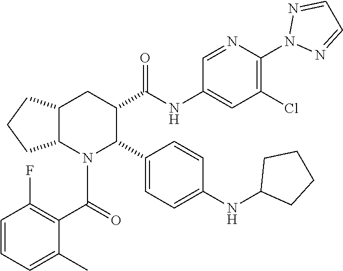

The title compound was synthesized in similar fashion as Example S63. ¹H NMR (400 MHz, METHANOL-d₄) δ 1.17-1.38 (3H, m), 1.54-1.84 (10H, m), 1.87-2.27 (6H, m), 2.39 (2 H, s), 3.17-3.26 (1H, m), 3.73-3.97 (2H, m), 6.53-6.70 (1H, m), 6.96-7.18 (2H, m), 7.20-7.31 (2H, m), 7.31-7.40 (1H, m), 7.74-7.83 (2H, m), 8.04 (2H, br d, J=7.09 Hz), 8.35-8.50 (1H, m). LC-MS: (ES) m/z 642.5 (M+H⁺).

### Example S93: Synthesis of cis-2-(4-(cyclopentylamino)phenyl)-N-(3-(dimethylphosphoryl)-4-methylphenyl)-1-(2-fluoro-6-methylbenzoyl)octahydro-1H-cyclopenta[b]pyridine-3-carboxamide (Compound No. 76)

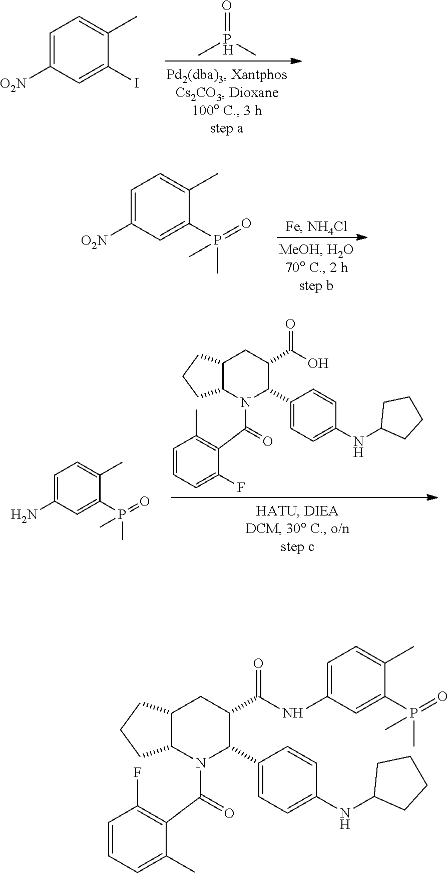

Step a) A solution of 2-iodo-1-methyl-4-nitro-benzene (0.5 g, 1.90 mmol), methylphos-phonoylmethane (296.73 mg, 3.80 mmol), Pd₂(dba)₃ (87.03 mg, 95.00 mol), Xantphos (109.99 mg, 190.00 mol) and Cs₂CO₃ (929.03 mg, 2.85 mmol) in dioxane (3 mL) was stirred at 90° C. for 3 h. The mixture was quenched by addition of H₂O (10 mL) and extracted with EtOAc (2×10 mL). The combined organic layers were dried, filtered and concentrated in vacuo to give the crude product. The crude product was purified by flash silica gel chromatography (ISCO®; 12 g SepaFlash® Silica Flash Column, eluent of 0˜30% ethyl acetate/petroleum ether gradient@25 mL/min, then 0˜0.05% MeOH/DCM gradient) to give 2-dimethylphosphoryl-1-methyl-4-nitro-benzene (0.2 g, 881.93 mol, 46.42% yield, 94% purity) as light orange solid. ¹H NMR (400 MHz, CDCl₃) δ 1.87 (s, 3H), 1.90 (s, 3H), 2.82 (s, 3H), 7.47 (dd, J=8.31, 3.67 Hz, 1H), 8.25 (dd, J=8.44, 1.59 Hz, 1H), 8.46 (dd, J=13.45, 2.20 Hz, 1H). LC-MS: (ES) m/z 214.1 (M+H⁺).

Step b) To a mixture of 2-dimethylphosphoryl-1-methyl-4-nitro-benzene (0.2 g, 938.22 mol) and NH₄Cl (100.37 mg, 1.88 mmol) in MeOH (8 mL)/H₂O (1.5 mL) was added Fe (209.58 mg, 3.75 mmol). Then the mixture was stirred at 70° C. for 16 h. The mixture was diluted with MeOH (30 mL) and filtered through a pad of Celite. The filtrate was concentrated in vacuo to give the crude product. The crude product was purified by prep-TLC (DCM/MeOH=8/1) to give 3-dimethylphosphoryl-4-methyl-aniline (0.1 g, 491.30 mol, 52.37% yield, 90% purity) as yellow semi-solid. LC-MS: (ES) m/z 184.2 (M+H⁺).

Step c) A solution of cis-2-[4-(cyclopentylamino)phenyl]-1-(2-fluoro-6-methyl-benzoyl)-2,3, 4,4a,5,6,7,7a-octahydrocyclopenta[b]pyridine-3-carboxylic acid (30 mg, 64.58 mol), HATU (29.46 mg, 77.49 mol) and DIEA (20.86 mg, 161.44 mol, 28.12 μL) in DCM (0.75 mL) was stirred at 30° C. for 0.5 h. Then 3-dimethylphosphoryl-4-methyl-aniline (15.77 mg, 77.49 mol) was added and the mixture was stirred at 30° C. for another 16 h. The mixture was concentrated in vacuo to give the crude product. The crude product was purified by prep-HPLC (column: Venusil ASB Phenyl 150×30 mm×5 μm; mobile phase: [water (0.05% HCl)-ACN]; B %: 37%-67%, 10 min). The compound cis-2-[4-(cyclopentylamino)phenyl]N-(3-dimethylpho sphoryl-4-methyl-phenyl)-1-(2-fluoro-6-methyl-benzoyl)-2,3,4,4a,5,6,7,7a-octahydrocyclopenta[b]pyridine-3-carboxamide (25 mg, 36.78 umol, 56.95% yield, 98% purity, HCl) was obtained as light yellow solid. ¹H NMR (400 MHz, METHANOL-d₄) δ 1.21-1.38 (m, 2H), 1.51-1.60 (m, 1H), 1.69-1.78 (m, 4 H), 1.85 (br s, 7H), 1.99-2.14 (m, 3H), 2.17-2.32 (m, 2H), 2.37-2.46 (m, 2H), 2.48-2.68 (m, 3H), 3.15-3.25 (m, 1H), 3.76-4.02 (m, 2H), 6.55-6.80 (m, 1H), 6.94-7.10 (m, 1H), 7.11-7.20 (m, 1H), 7.29 (br d, J=8.28 Hz, 1H), 7.33-7.41 (m, 1H), 7.45 (br d, J=8.28 Hz, 2H), 7.55-7.71 (m, 1H), 7.76-7.97 (m, 3H). LC-MS: (ES) m/z 630.3 (M+H⁺).

### Example S94: Synthesis of Synthesis of cis-2-(4-(cyclopentylamino)phenyl)-1-(2-fluoro-6-methylbenzoyl)-N-(2-methyl-1,2,3,4-tetrahydroisoquinolin-6-yl)octahydro-1H-cyclopenta[b]pyridine-3-carboxamide (Compound No. 77)

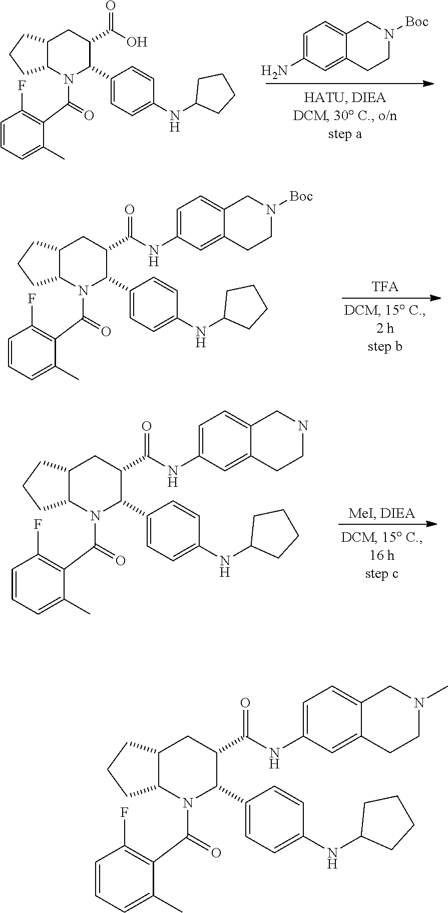

Step a) A solution of cis-2-[4-(cyclopentylamino)phenyl]-1-(2-fluoro-6-methyl-benzoyl)-2,3,4,4a,5,6,7,7a-octahydrocyclopenta[b]pyridine-3-carboxylic acid (0.1 g, 182.96 mol), HATU (83.48 mg, 219.56 mol) and DIEA (59.12 mg, 457.41 mol, 79.67 μL) in DCM (1 mL) was stirred at 30° C. for 0.5 h. Then tert-butyl6-amino-3,4-dihydro-1H-iso quinoline-2-carboxylate (54.52 mg, 219.56 mol) was added and the mixture was stirred at 30° C. for another 16 h. The mixture was concentrated in vacuo to give the crude. The crude was purified by silica gel column chromatography to give cis-tert-butyl-6-[[2-[4-(cyclopentylamino) phenyl]-1-(2-fluoro-6-methyl-benzoyl)-2,3,4,4a,5,6,7,7a-octahydrocyclopenta[b]pyridine-3-carbonyl]amino]-3,4-dihydro-1H-isoquinoline-2-carboxylate (60 mg, 82.89 mol, 45.31% yield, 96% purity) as light yellow solid. ¹H NMR (400 MHz, METHANOL-d₄) δ1.23-1.39 (m, 3H), 1.46-1.50 (m, 13H), 1.55-1.64 (m, 3 H), 1.66-1.77 (m, 3H), 1.96-2.04 (m, 2H), 2.06-2.22 (m, 2H), 2.27 (s, 1H), 2.33-2.44 (m, 2H), 2.71-2.83 (m, 2H), 2.97-3.12 (m, 1H), 3.56-3.64 (m, 2H), 3.68-3.87 (m, 2 H), 4.49 (br s, 2H), 6.51-6.62 (m, 3H), 6.99-7.08 (m, 2H), 7.10-7.17 (m, 1H), 7.18-7.29 (m, 2H), 7.30-7.34 (m, 1H), 7.39 (d, J=8.53 Hz, 1H), 7.45 (d, J=8.78 Hz, 1H). LC-MS: (ES) m/z 695.5 (M+H⁺).

Step b) To a solution of cis-tert-butyl6-[[2-[4-(cyclopentylamino)phenyl]-1-(2-fluoro-6-methyl-benzoyl)-2,3,4,4a,5,6,7,7a-octahydrocyclopenta[b]pyridine-3-carbonyl]amino]-3,4-dihydro-1H-isoquinoline-2-carboxylate (55 mg, 79.15 mol) in DCM (2 mL) was added TFA (264.69 mg, 2.32 mmol, 171.87 μL). Then the mixture was stirred at 15° C. for 2 h. The mixture was concentrated in vacuo to give the crude. The crude was dissolved with DCM (20 mL) and alkalified to pH=8-9 by addition of saturated NaHCO₃ solution. The organic layer separated was dried, filtered and concentrated in vacuo to give the desired product cis-2-[4-(cyclopentylamino) phenyl]-1-(2-fluoro-6-methyl-benzoyl)-N-(1,2,3,4-tetrahydroisoquinolin-6-yl)-2,3,4,4a,5,6,7,7a-octahydrocyclopenta[b]-pyridine-3-carboxamide (48 mg, 75.06 mol, 94.83% yield, 93% purity) as white solid. ¹H NMR (400 MHz, METHANOL-d₄) δ1.27-1.37 (m, 3H), 1.49 (br d, J=6.85 Hz, 3H), 1.58-1.64 (m, 3 H), 1.68-1.75 (m, 3H), 1.97-2.06 (m, 3H), 2.12-2.22 (m, 2H), 2.28 (s, 1H), 2.40 (s, 2 H), 2.79 (br d, J=4.89 Hz, 3H), 2.97-3.11 (m, 4H), 3.69-3.79 (m, 2H), 3.84-3.95 (m, 3 H), 6.54-6.62 (m, 3H), 6.94-7.08 (m, 2H), 7.12-7.21 (m, 2H), 7.22-7.29 (m, 1H), 7.35-7.42 (m, 2H), 7.46 (d, J=8.80 Hz, 1H). LC-MS: (ES) m/z 595.5 (M+H⁺).

Step c) To a solution of cis-2-[4-(cyclopentylamino)phenyl]-1-(2-fluoro-6-methyl-benzoyl)-N-(1,2,3,4-tetrahydroisoquinolin-6-yl)-2,3,4,4a,5,6,7,7a-octahydrocyclopenta[b]pyridine-3-carboxamide (34 mg, 57.17 mol) and DIEA (14.78 mg, 114.33 mol, 19.91 μL) in DCM (2 mL) was added dropwise of a solution of MeI (6.49 mg, 45.73 mol, 2.85 μL) in DCM (1 mL). Then the mixture was stirred at 15° C. for 16 h. The mixture was concentrated in vacuo to give the residue. The residue was purified by prep-TLC (DCM/MeOH=10/1) to give cis-2-[4-(cyclopentyl amino)phenyl]-1-(2-fluoro-6-methyl-benzoyl)-N-(2-methyl-3,4-dihydro-1H-isoquinolin-6-yl)-2,3,4,4a,5,6,7,7a-octahydrocyclopenta[b]pyridine-3-carboxamide (3 mg, 4.53 umol, 7.92% yield, 92% purity) as off-white solid. ¹H NMR (400 MHz, METHANOL-d₄) δ1.48 (br d, J=9.54 Hz, 3H), 1.56-1.66 (m, 4H), 1.68-1.77 (m, 3H), 1.91-2.07 (m, 4H), 2.10-2.23 (m, 2H), 2.27 (s, 1H), 2.33-2.45 (m, 3H), 2.46-2.51 (m, 3H), 2.72-2.81 (m, 2H), 2.91 (q, J=7.09 Hz, 2H), 3.03 (br d, J=10.76 Hz, 1H), 3.53-3.62 (m, 2H), 3.67-3.89 (m, 2H), 6.50-6.61 (m, 3H), 6.89-7.04 (m, 2H), 7.05-7.20 (m, 2H), 7.21-7.27 (m, 1H), 7.29-7.38 (m, 2H), 7.44 (d, J=8.80 Hz, 1H). LC-MS: (ES) m/z 609.4 (M+H⁺).

### Example S95: Synthesis of cis-2-(4-(cyclopentylamino)phenyl)-1-(2-fluoro-6-methylbenzoyl)-N-(1-methyl-1H-pyrazolo[4,3-b]pyridin-6-yl)octahydro-1H-cyclopenta[b]pyridine-3-carboxamide (Compound No. 78)

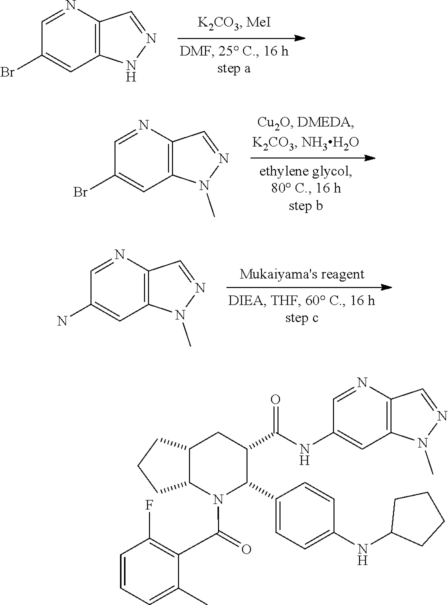

Step a) The K₂CO₃ (1.40 g, 10.10 mmol) and MeI (716.79 mg, 5.05 mmol, 314.38 μL) were added to a solution of 6-bromo-1H-pyrazolo[4,3-b]pyridine (0.5 g, 2.52 mmol) in DMF (10 mL). The mixture was stirred at 25° C. for 16 h. The reaction mixture was added H₂O (20 mL) and was extracted with EtOAc (30 mL×2). The combined organic layers were washed with brine (25 mL), dried with anhydrous Na₂SO₄, filtered, then the filtrate was evaporated under vacuum to give a residue. The residue was purified by column chromatography (SiO₂, petroleum ether/ethyl acetate=100/0 to 3/1) to give compound 6-bromo-1-methyl-pyrazolo[4,3-b]pyridine (180 mg, 840.38 μmol, 33.28% yield, 99% purity) as a pale yellow solid. ¹H NMR (400 MHz, CDCl₃) δ 4.07 (3 H, s) 7.94 (1H, d, J=1.51 Hz) 8.20 (1 H, s) 8.60 (1H, d, J=1.76 Hz). LCMS: m/z 214.0 (M+H⁺).

Step b) The NH₃·H₂O (2.10 g, 16.81 mmol, 2.31 mL, 28% purity) and Cu₂O (60.13 mg, 420.19 mol, 42.95 μL) were added to a mixture of K₂CO₃ (116.15 mg, 840.38 mol), DMEDA (37.04 mg, 420.19 mol, 45.23 μL) and 6-bromo-1-methyl-pyrazolo[4,3-b]pyridine (180 mg, 840.38 mol) in ethylene glycol (10 mL) The mixture was stirred at 80° C. for 16 h. The reaction mixture was added H₂O (10 mL) and was extracted with DCM:MeOH=10:1 (20 mL×2). The combined organic layers were washed with brine (10 mL), dried with anhydrous Na₂SO₄, filtered, then the filtrate was evaporated under vacuum to give crude product. The crude product was purified by prep-TLC (plate: DCM:CH₃₀H=15:1) to give compound 1-methylpyrazolo[4,3-b]pyridin-6-amine (40 mg, 261.87 mol, 31.16% yield, 97% purity) as a pale yellow solid. ¹H NMR (400 MHz, CDCl₃) δ 3.96 (3 H, s) 6.84 (1H, d, J=1.25 Hz) 8.04 (1 H, s) 8.13 (1 H, br s). LCMS: m/z 149.2 (M+H⁺).

Step c) The 2-[4-(cyclopentylamino)phenyl]-1-(2-fluoro-6-methyl-benzoyl)-2,3,4,4a,5,6,7,7a-octahydrocyclopenta[b]pyridine-3-carboxylicacid (20 mg, 43.05 mol) and 1-methylpyrazolo[4,3-b]pyridin-6-amine (7.65 mg, 51.66 mol) were dissolved in THF (2 mL). Then 2-chloro-1-methyl-pyridin-1-ium; iodide (16.50 mg, 64.58 mol) and DIEA (16.69 mg, 129.15 mol, 22.50 μL) were added. The mixture was stirred at 60° C. for 16 h. The solvent was evaporated under vacuum to give crude product. The crude product was purified by prep-HPLC (column: Venusil ASB Phenyl 150×30 mm×5 μm; mobile phase: [water (0.05% HCl)-ACN]; B %: 45%-75%, 9 min) to give cis-2-[4-(cyclopentylamino)phenyl]-1-(2-fluoro-6-methyl-benzoyl)-N-(1-methylpyrazolo[4,3-b]pyridin-6-yl)-2,3,4,4a,5,6,7,7a-octahydrocyclopenta[b]pyridine-3-carboxamide (8.5 mg, 13.47 mol, 31.28% yield, 100% purity, HCl) as a yellow solid. ¹H NMR (400 MHz, METHANOL-d₄) δ 1.15-1.44 (3H, m) 1.46-2.08 (13H, m) 2.11-2.40 (4H, m) 2.44 (2 H, s) 3.80-4.08 (2H, m) 4.11-4.19 (3H, m) 6.66-6.84 (1H, m) 7.00-7.14 (1H, m) 7.15-7.30 (1H, m) 7.36-7.45 (1H, m) 7.48 (2H, d, J=8.53 Hz) 7.88-8.01 (2H, m) 8.15-8.28 (1H, m) 8.63-8.86 (2H, m). LCMS: m/z 595.5 (M+H⁺).

### Example S96: Synthesis of cis-2-(4-(cyclopentylamino)phenyl)-1-(2-fluoro-6-methylbenzoyl)-N-(2-(trifluor omethyl)pyridin-4-yl)octahydro-1H-cyclopenta[b]pyridine-3-carboxamide (Compound No. 79)

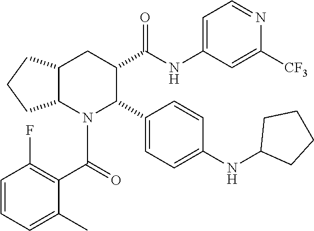

The title compound was synthesized in similar fashion as Example S95. ¹H NMR (400 MHz, METHANOL-d₄) δ1.23-1.34 (m, 2H), 1.51-1.61 (m, 1H), 1.67 (br s, 5H), 1.82 (br s, 3H), 1.86-2.00 (m, 3H), 2.01-2.12 (m, 2H), 2.12-2.21 (m, 2H), 2.22-2.30 (m, 1H), 2.35-2.44 (m, 2H), 3.19-3.28 (m, 1H), 3.74-4.02 (m, 2H), 6.54-6.70 (m, 1 H), 7.01-7.09 (m, 1H), 7.11-7.19 (m, 1H), 7.31-7.43 (m, 3H), 7.66-7.78 (m, 1H), 7.82-7.91 (m, 2H), 8.04-8.15 (m, 1H), 8.41-8.57 (m, 1H). LC-MS: (ES) m/z 609.3 (M+H⁺).

### Example S97: Synthesis of (2R,3S,4aR,7aR)-2-(4-(cyclopentylamino)phenyl)-1-(2-fluoro-6-methylbenzoyl)-N-(1-(pyridin-2-ylmethyl)-1H-indazol-5-yl)octahydro-1H-cyclopenta[b]pyridine-3-carboxamide (Compound No. 80)

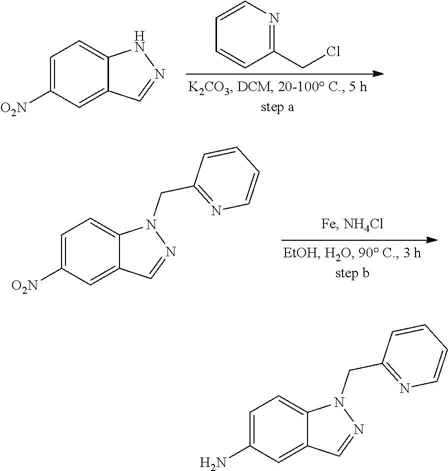 [SMILES: O=[N+]([O-])c1ccc2[nH]ncc2c1 ; ClCc1ccccn1 ; O=[N+]([O-])c1ccc2c(cnn2Cc2ccccn2)c1 ; Nc1ccc2c(cnn2Cc2ccccn2)c1]

Step a) To a mixture of 5-nitro-1H-indazole (2 g, 12.26 mmol) and 2-(chloromethyl) pyridine (2.41 g, 14.71 mmol, HCl) in DMF (10 mL) was added K₂CO₃ (4.24 g, 30.65 mmol) at 20° C. under N₂. The mixture was stirred at 100° C. for 5 h. The reaction mixture was partitioned between EtOAc 100 mL and H₂O 100 mL. The organic phase was separated, dried, filtered and concentrated under reduced pressure to give a residue. The residue was purified by column chromatography (SiO₂, petroleum ether/ethyl acetate=1/1 to 1/1). Compound 5-nitro-1-(2-pyridylmethyl)indazole (640 mg, 2.37 mmol, 19.30% yield, 94% purity) was obtained as a yellow solid. ¹H NMR (400 MHz, CHLOROFORM-d) δ ppm 5.75 (s, 2H), 7.00 (d, J=7.83 Hz, 1H), 7.20 (dd, J=6.97, 5.26 Hz, 1H), 7.52 (d, J=9.05 Hz, 1 H), 7.60 (td, J=7.70, 1.71 Hz, 1H), 8.22 (dd, J=9.29, 2.20 Hz, 1H), 8.25 (d, J=0.73 Hz, 1H), 8.56 (d, J=4.16 Hz, 1H), 8.72 (d, J=1.71 Hz, 1H) LCMS: (ES) m/z 255.1 (M+H⁺).

Step b) A mixture of 5-nitro-1-(2-pyridylmethyl)indazole (400 mg, 1.57 mmol), Fe (702.88 mg, 12.59 mmol) and NH₄Cl (42.08 mg, 786.65 mol) in EtOH (10 mL) and H₂O (2.5 mL) was degassed and purged with N₂ for 3 times, and then the mixture was stirred at 90° C. for 3 h under N₂ atmosphere, filtered, then concentrated to get the desired product. Compound 1-(2-pyridylmethyl) indazol-5-amine (350 mg, 1.56 mmol, 99.20% yield) was obtained as a yellow solid. ¹H NMR (400 MHz, CDCl₃) δ ppm 5.62-5.76 (m, 2H) 6.76-6.87 (m, 2H) 6.96 (d, J=1.55 Hz, 1H) 7.13-7.25 (m, 2H) 7.54 (td, J=7.72, 1.61 Hz, 1H) 7.88 (s, 1H) 8.58 (d, J=4.53 Hz, 1H) LCMS: (ES) m/z 225.4 (M+H⁺).

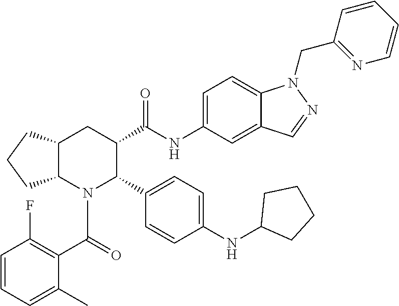 [SMILES: Cc1cccc(F)c1C(=O)N1[C@@H]2CCC[C@@H]2C[C@H](C(=O)Nc2ccc3c(cnn3Cc3ccccn3)c2)[C@@H]1c1ccc(NC2CCCC2)cc1]

The title compound was synthesized in similar fashion as Example S95. ¹H NMR (400 MHz, METHANOL-d₄) δ ppm 1.25-1.41 (m, 4H) 1.69 (br s, 3H) 1.85 (br s, 4H) 1.91-2.03 (m, 4H) 2.06-2.23 (m, 4H) 2.24-2.37 (m, 4H) 2.44 (s, 2H) 3.21-3.29 (m, 1H) 3.69 (br t, J=12.05 Hz, 2H) 3.80-4.02 (m, 2H) 4.13 (br d, J=7.28 Hz, 2H) 4.73-4.85 (m, 1H) 6.57-6.72 (m, 1H) 7.04-7.12 (m, 1H) 7.14-7.23 (m, 1H) 7.35-7.46 (m, 4H) 7.58-7.64 (m, 1H) 7.90 (br d, J=8.78 Hz, 2H) 7.95-7.98 (m, 2H). LCMS: (ES) m/z 664.3 (M+H⁺).

### Example S98: Synthesis of (2R,3S,4aR,7aR)-2-(4-(cyclopentylamino)phenyl)-1-(2-fluoro-6-methylbenzoyl)-N-(1-(tetrahydro-2H-pyran-4-yl)-1H-indazol-5-yl)octahydro-1H-cyclopenta[b]pyridine-3-carboxamide (Compound No. 81)

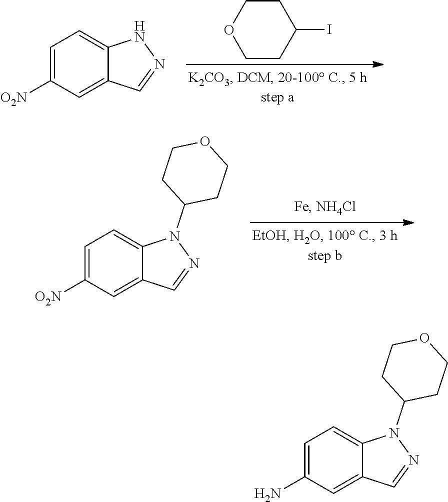 [SMILES: O=[N+]([O-])c1ccc2[nH]ncc2c1 ; IC1CCOCC1 ; O=[N+]([O-])c1ccc2c(cnn2C2CCOCC2)c1 ; Nc1ccc2c(cnn2C2CCOCC2)c1]

Step a) To a mixture of 5-nitro-1H-indazole (2 g, 12.26 mmol) and 1-methylpiperidin-4-ol (2.12 g, 18.39 mmol, 2.15 mL), PPh₃ (4.82 g, 18.39 mmol) in THF (20 mL) was added DIAD (1.96 M in toluene) (1.9 M, 9.68 mL) at 0° C. under N₂. The mixture was stirred at 25° C. for 16 h. The reaction solvent was concentrated to get a residue. The residue was purified by prep-HPLC:column: Welch Xtimate C18 150*40 mm*10 μm; mobile phase: [water (10 mM NH₄HCO₃)-ACN]; B %: 36%-46%, 8 min to give 1-(1-methyl-4-piperidyl)-5-nitro-indazole (900 mg, 3.46 mmol, 28.20% yield, 100% purity) was obtained as a light yellow solid. ¹H NMR (400 MHz, CDCl₃) δ ppm 1.98-2.05 (m, 3H) 2.15-2.27 (m, 2H) 2.35-2.51 (m, 5H) 3.06 (br d, J=12.05 Hz, 2H) 4.46 (tt, J=11.61, 4.20 Hz, 1H) 7.53 (d, J=9.29 Hz, 1H) 8.21 (s, 1H) 8.25 (dd, J=9.16, 2.13 Hz, 1H) 8.72 (d, J=2.01 Hz, 1 H) LCMS: (ES) m/z 261.3 (M+H⁺).

Step b) A mixture of 1-(1-methyl-4-piperidyl)-5-nitro-indazole (400 mg, 1.54 mmol), NH₄Cl (41.10 mg, 768.37 mol) and Fe (686.55 mg, 12.29 mmol) in EtOH (10 mL) and H₂O (2.5 mL) was degassed and purged with N₂ for 3 times, and then the mixture was stirred at 90° C. for 3 h under N₂ atmosphere, filtered, then concentrated to get the desired product. Compound 1-(1-methyl-4-piperidyl)indazol-5-amine (300 mg, 1.30 mmol, 84.76% yield) was obtained as a yellow solid. ¹H NMR (400 MHz, CDCl₃) δ ppm 2.15 (br s, 1H) 2.26-2.41 (m, 4H) 2.44 (br s, 3H) 3.14 (br s, 2H) 3.40-3.76 (m, 1H) 4.41 (br s, 1H) 6.86 (dd, J=8.91, 2.13 Hz, 1H) 6.95 (d, J=1.76 Hz, 1H) 7.31 (d, J=8.78 Hz, 1H) 7.78 (s, 1H) LC-MS: (ES) m/z 231.3 (M+H⁺).

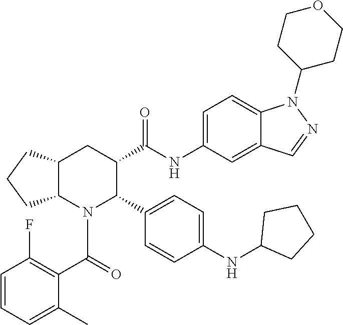 [SMILES: Cc1cccc(F)c1C(=O)N1[C@@H]2CCC[C@@H]2C[C@H](C(=O)Nc2ccc3c(cnn3C3CCOCC3)c2)[C@@H]1c1ccc(NC2CCCC2)cc1]

The title compound was synthesized in similar fashion as Example S95. ¹H NMR (400 MHz, METHANOL-d₄) δ ppm 1.21-1.33 (m, 2H) 1.49-1.59 (m, 1H) 1.70 (br s, 6H) 1.85 (br s, 3H) 1.98 (br d, J=17.57 Hz, 3H) 2.08-2.17 (m, 2H) 2.17-2.24 (m, 2H) 2.28 (br d, J=13.55 Hz, 3H) 2.44 (s, 2H) 2.47-2.56 (m, 2H) 2.92-3.04 (m, 4H) 3.25 (br dd, J=10.16, 5.90 Hz, 2H) 3.72 (brd, J=12.80 Hz, 2H) 3.78-3.88 (m, 1H) 3.93-4.02 (m, 1H) 6.58-6.74 (m, 1H) 7.04-7.12 (m, 1H) 7.14-7.23 (m, 1H) 7.37-7.50 (m, 4H) 7.57-7.67 (m, 1 H) 7.88-8.05 (m, 4H) LCMS: (ES) m/z 677.4 (M+H⁺).

### Example S99: Synthesis of (2R,3S,4aR,7aR)-2-(4-(cyclopentylamino)phenyl)-1-(2-fluoro-6-methylbenzoyl)-N-(1-(1-methylpiperidin-4-yl)-1H-indazol-5-yl)octahydro-1H-cyclopenta[b]pyridine-3-carboxamide (Compound No. 82)

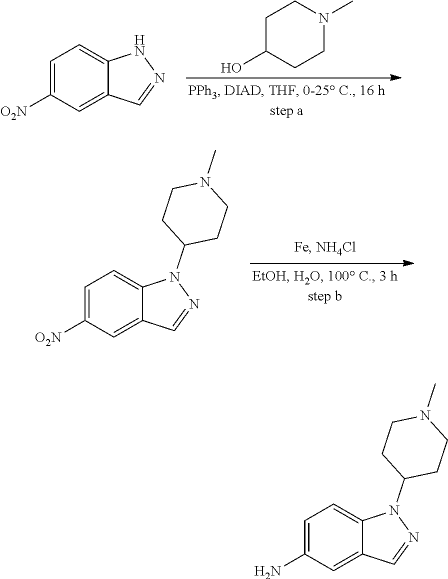

Step a) To a mixture of 5-nitro-1H-indazole (2 g, 12.26 mmol) and 1-methylpiperidin-4-ol (2.12 g, 18.39 mmol, 2.15 mL), PPh₃ (4.82 g, 18.39 mmol) in THF (20 mL) was added DIAD (1.96 M in toluene) (1.9 M, 9.68 mL) at 0° C. under N₂. The mixture was stirred at 25° C. for 16 h. The reaction solvent was concentrated to get a residue. The residue was purified by prep-HPLC: column: Welch Xtimate C18 150*40 mm*10 μm; mobile phase: [water (10 mM NH₄HCO₃)-ACN]; B %: 36%-46%, 8 min to give 1-(1-methyl-4-piperidyl)-5-nitro-indazole (900 mg, 3.46 mmol, 28.20% yield, 100% purity) was obtained as a light yellow solid. ¹H NMR (400 MHz, CDCl₃) δ ppm 1.98-2.05 (m, 3H) 2.15-2.27 (m, 2H) 2.35-2.51 (m, 5H) 3.06 (br d, J=12.05 Hz, 2H) 4.46 (tt, J=11.61, 4.20 Hz, 1H) 7.53 (d, J=9.29 Hz, 1H) 8.21 (s, 1H) 8.25 (dd, J=9.16, 2.13 Hz, 1H) 8.72 (d, J=2.01 Hz, 1 H) LCMS: (ES) m/z 261.3 (M+H⁺).

Step b) A mixture of 1-(1-methyl-4-piperidyl)-5-nitro-indazole (400 mg, 1.54 mmol), NH₄Cl (41.10 mg, 768.37 mol) and Fe (686.55 mg, 12.29 mmol) in EtOH (10 mL) and H₂O (2.5 mL) was degassed and purged with N₂ for 3 times, and then the mixture was stirred at 90° C. for 3 h under N₂ atmosphere, filtered, then concentrated to get the desired product. Compound 1-(1-methyl-4-piperidyl)indazol-5-amine (300 mg, 1.30 mmol, 84.76% yield) was obtained as a yellow solid. ¹H NMR (400 MHz, CDCl₃) δ ppm 2.15 (br s, 1H) 2.26-2.41 (m, 4H) 2.44 (br s, 3H) 3.14 (br s, 2H) 3.40-3.76 (m, 1H) 4.41 (br s, 1H) 6.86 (dd, J=8.91, 2.13 Hz, 1H) 6.95 (d, J=1.76 Hz, 1H) 7.31 (d, J=8.78 Hz, 1H) 7.78 (s, 1H) LC-MS: (ES) m/z 231.3 (M+H⁺).

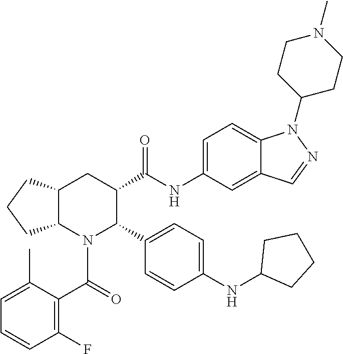 [SMILES: Cc1cccc(F)c1C(=O)N1[C@@H]2CCC[C@@H]2C[C@H](C(=O)Nc2ccc3c(cnn3C3CCN(C)CC3)c2)[C@@H]1c1ccc(NC2CCCC2)cc1]

The title compound was synthesized in similar fashion as Example S95. ¹H NMR (400 MHz, METHANOL-d₄) δ ppm 1.21-1.33 (m, 2H) 1.49-1.59 (m, 1H) 1.70 (br s, 6H) 1.85 (br s, 3H) 1.98 (br d, J=17.57 Hz, 3H) 2.08-2.17 (m, 2H) 2.17-2.24 (m, 2H) 2.28 (br d, J=13.55 Hz, 3H) 2.44 (s, 2H) 2.47-2.56 (m, 2H) 2.92-3.04 (m, 4H) 3.25 (br dd, J=10.16, 5.90 Hz, 2H) 3.72 (brd, J=12.80 Hz, 2H) 3.78-3.88 (m, 1H) 3.93-4.02 (m, 1H) 6.58-6.74 (m, 1H) 7.04-7.12 (m, 1H) 7.14-7.23 (m, 1H) 7.37-7.50 (m, 4H) 7.57-7.67 (m, 1 H) 7.88-8.05 (m, 4H) LCMS: (ES) m/z 677.4 (M+H⁺).

### Example S100: Synthesis of (2R,3S,4aR,7aR)-2-(4-(cyclopentylamino)phenyl)-1-(2-fluoro-6-methylbenzoyl)-N-(1-(oxetan-3-yl)-1H-indazol-5-yl)octahydro-1H-cyclopenta[b]pyridine-3-carboxamide (Compound No. 83)

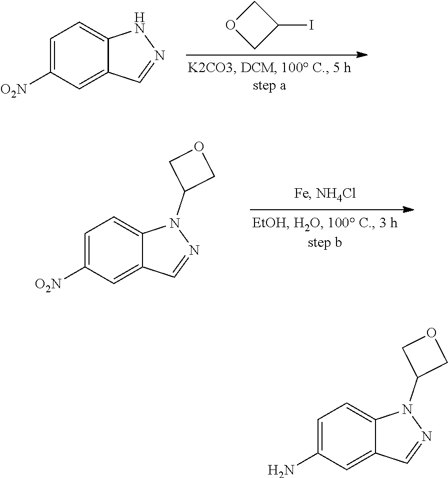 [SMILES: O=[N+]([O-])c1ccc2[nH]ncc2c1 ; IC1COC1 ; O=[N+]([O-])c1ccc2c(cnn2C2COC2)c1 ; Nc1ccc2c(cnn2C2COC2)c1]

Step a) To a mixture of 5-nitro-1H-indazole (2 g, 12.26 mmol) and 3-iodooxetane (2.71 g, 14.71 mmol) in DMF (10 mL) was added K₂CO₃ (3.39 g, 24.52 mmol) at 20° C. under N₂. The mixture was stirred at 100° C. for 5 h. The reaction mixture was partitioned between EtOAc 100 mL and H₂O 100 mL. The organic phase was separated, dried, filtered and concentrated under reduced pressure to give a residue. The residue was purified by prep-HPLC: (column: YMC-Triart Prep C18 150*40 mm*7 m; mobile phase: [water (0.04% NH₃H₂O+10 mM NH₄HCO₃)-ACN]; B %: 32%-42%, 10 min) The compound 5-nitro-1-(oxetan-3-yl)indazole (350 mg, 1.56 mmol, 98% purity) was obtained as yellow solid. ¹H NMR (400 MHz, CDCl₃) δ ppm 5.13-5.23 (m, 2H), 5.30 (t, J=6.65 Hz, 2H), 5.76-5.92 (m, 1H), 7.61 (d, J=9.29 Hz, 1H), 8.26-8.37 (m, 2H), 8.76 (d, J=2.01 Hz, 1H) LCMS: (ES) m/z 220.1 (M+H⁺).

Step b) A mixture of 5-nitro-1-(oxetan-3-yl)indazole (400 mg, 1.82 mmol), NH₄Cl (48.81 mg, 912.43 mol) and Fe (815.27 mg, 14.60 mmol) in EtOH (10 mL) and H₂O (2.5 mL) was degassed and purged with N₂ for 3 times, and then the mixture was stirred at 90° C. for 3 h under N₂ atmosphere. Filtered, then concentrated to get the desired product. Compound 1-(oxetan-3-yl) indazol-5-amine (335 mg, 1.77 mmol, 97.02% yield) was obtained as a yellow solid. ¹H NMR (400 MHz, CDCl₃) δ ppm 5.07-5.18 (m, 2H) 5.28 (br t, J=6.40 Hz, 2H) 5.72 (quin, J=6.90 Hz, 1H) 6.90 (br d, J=8.78 Hz, 1H) 6.96 (s, 1H) 7.37 (br d, J=8.78 Hz, 1H) 7.88 (s, 1H). LCMS: (ES) m/z 190.1 (M+H⁺).

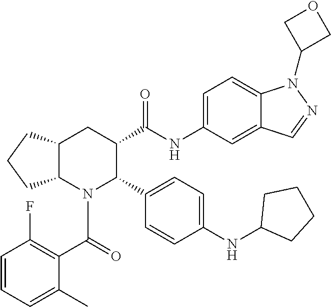 [SMILES: Cc1cccc(F)c1C(=O)N1[C@@H]2CCC[C@@H]2C[C@H](C(=O)Nc2ccc3c(cnn3C3COC3)c2)[C@@H]1c1ccc(NC2CCCC2)cc1]

The title compound was synthesized in similar fashion as Example S95. ¹H NMR (400 MHz, METHANOL-d₄) δ ppm 1.31 (br s, 2H) 1.42-1.66 (m, 6H) 1.73 (br s, 2H) 1.97 (br d, J=11.29 Hz, 3H) 2.08-2.27 (m, 3H) 2.30 (s, 1H) 2.39-2.46 (m, 2H) 3.10 (br s, 1H) 3.76 (br s, 2H) 5.07-5.25 (m, 6H) 5.96 (br d, J=6.27 Hz, 1H) 6.58-6.67 (m, 3H) 7.04-7.19 (m, 2H) 7.41-7.60 (m, 4H) 7.86-8.11 (m, 2H) LCMS: (ES) m/z 636.3 (M+H⁺).

### Example S101: Synthesis of (2R,3S,4aR,7aR)-2-(4-(cyclopentylamino)phenyl)-1-(2-fluoro-6-methylbenzoyl)-N-(JH-indazol-5-yl)octahydro-1H-cyclopenta[b]pyridine-3-carboxamide (Compound No. 84)

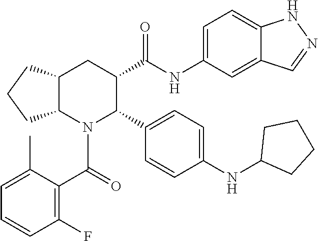 [SMILES: Cc1cccc(F)c1C(=O)N1[C@@H]2CCC[C@@H]2C[C@H](C(=O)Nc2ccc3[nH]ncc3c2)[C@@H]1c1ccc(NC2CCCC2)cc1]

The title compound was synthesized in similar fashion as Example S95. ¹H NMR (400 MHz, METHANOL-d₄) δ ppm 1.24-1.43 (m, 3H) 1.70 (br d, J=4.52 Hz, 6H) 1.85 (br s, 4H) 1.93-2.07 (m, 3H) 2.07-2.18 (m, 2H) 2.37-2.54 (m, 3H) 3.18-3.30 (m, 1H) 3.73-3.92 (m, 1H) 3.93-4.06 (m, 1H) 6.57-6.73 (m, 1H) 7.03-7.12 (m, 1H) 7.13-7.22 (m, 1H) 7.34-7.57 (m, 6H) 7.89-7.99 (m, 3H) 8.00-8.04 (m, 1H) LCMS: (ES) m/z 580.3 (M+H⁺).

### Example S102: Synthesis of (2R,3S,4aR,7aR)-2-(4-(cyclopentylamino)phenyl)-1-(2-fluoro-6-methylbenzoyl)-N-(1-methyl-1H-indol-5-yl)octahydro-1H-cyclopenta[b]pyridine-3-carboxamide (Compound No. 85)

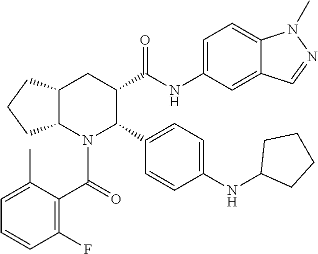 [SMILES: Cc1cccc(F)c1C(=O)N1[C@@H]2CCC[C@@H]2C[C@H](C(=O)Nc2ccc3c(cnn3C)c2)[C@@H]1c1ccc(NC2CCCC2)cc1]

The title compound was synthesized in similar fashion as Example S95. ¹H NMR (400 MHz, METHANOL-d₄) δ ppm 1.32 (br d, J=13.05 Hz, 4H) 1.44-1.56 (m, 2H) 1.67 (brs, 6H) 1.82 (brs, 4H) 1.89-2.06 (m, 3H) 2.11 (s, 1H) 2.20-2.29 (m, 2H) 2.35-2.51 (m, 2H) 3.19 (br d, J=14.56 Hz, 1H) 3.72-3.79 (m, 1H) 3.93 (s, 1H) 6.34-6.40 (m, 1H) 6.56-6.70 (m, 1H) 7.04-7.25 (m, 6H) 7.26-7.34 (m, 1H) 7.36-7.44 (m, 1H) 7.56-7.67 (m, 1H) 7.80 (br s, 2H) LCMS: (ES) m/z 593.3 (M+H⁺).

### Example S103: Synthesis of (2R,3S,4aR,7aR)-2-(4-(cyclopentylamino)phenyl)-1-(2-fluoro-6-methylbenzoyl)-N-(1-(pyridin-3-ylmethyl)-1H-indazol-5-yl)octahydro-1H-cyclopenta[b]pyridine-3-carboxamide (Compound No. 86)

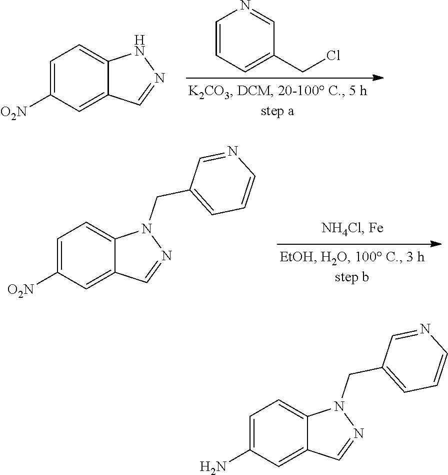 [SMILES: O=[N+]([O-])c1ccc2[nH]ncc2c1 ; ClCc1cccnc1 ; O=[N+]([O-])c1ccc2c(cnn2Cc2cccnc2)c1 ; Nc1ccc2c(cnn2Cc2cccnc2)c1]

Step a) To a mixture of 5-nitro-1H-indazole (2 g, 12.26 mmol) and 3-(chloromethyl)-pyridine (2.41 g, 14.71 mmol, HCl) in DMF (10 mL) was added K₂CO₃ (5.08 g, 36.78 mmol) at 20° C. under N₂. The mixture was stirred at 100° C. for 5 h. The reaction mixture was partitioned between EtOAc 100 mL and H₂O 100 mL. The organic phase was separated, dried, filtered and concentrated under reduced pressure to give a residue. The residue was purified by Prep-HPLC: column: Welch Xtimate C18 150*40 mm*10 μm; mobile phase: [water (10 mM NH₄HCO₃)-ACN]; B %: 31%-51%, 8 min to give 5-nitro-1-(3-pyridylmethyl)indazole (300 mg, 1.18 mmol, 9.62% yield) was obtained as a yellow solid. ¹H NMR (400 MHz, CDCl₃) δ ppm 5.49-5.79 (m, 2H) 7.25-7.31 (m, 2H) 7.46 (d, J=9.03 Hz, 1H) 7.51-7.58 (m, 1H) 8.26-8.33 (m, 2H) 8.57-8.65 (m, 2H) 8.77 (d, J=1.76 Hz, 1H) LCMS: (ES) m/z 255.1 (M+H⁺).

Step b) A mixture of 5-nitro-1-(3-pyridylmethyl)indazole (300 mg, 1.18 mmol), NH₄Cl (31.56 mg, 589.99 mol) and Fe (527.16 mg, 9.44 mmol) in EtOH (10 mL) and H₂O (2.5 mL) was degassed and purged with N₂ for 3 times, and then the mixture was stirred at 90° C. for 3 h under N₂ atmosphere, filtered, then concentrated to get the desired product. Compound 1-(3-pyridylmethyl)-indazol-5-amine (220 mg, 981.00 mol, 83.14% yield) was obtained as a yellow solid. ¹H NMR (400 MHz, CDCl₃) δ ppm 5.55 (s, 2H) 6.85 (dd, J=8.78, 2.01 Hz, 1H) 6.96 (d, J=1.76 Hz, 1H) 7.13-7.25 (m, 2H) 7.44 (br d, J=8.03 Hz, 1H) 7.85 (s, 1H) 8.50-8.53 (m, 1H) 8.56 (d, J=1.51 Hz, 1H) LCMS: (ES) m/z 225.4 (M+H⁺).

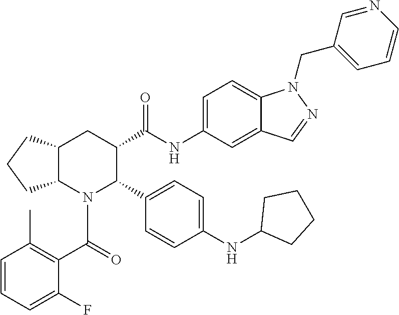 [SMILES: Cc1cccc(F)c1C(=O)N1[C@@H]2CCC[C@@H]2C[C@H](C(=O)Nc2ccc3c(cnn3Cc3cccnc3)c2)[C@@H]1c1ccc(NC2CCCC2)cc1]

The title compound was synthesized in similar fashion as Example S95. ¹H NMR (400 MHz, METHANOL-d₄) δ ppm 1.19-1.40 (m, 3H) 1.45-1.63 (m, 2H) 1.64-1.78 (m, 6H) 1.84 (br d, J=11.04 Hz, 3H) 2.00 (br s, 2H) 2.08-2.18 (m, 2H) 2.21-2.34 (m, 2H) 2.44 (s, 2H) 3.21-3.27 (m, 1H) 3.80-4.04 (m, 2H) 5.91-6.01 (m, 2H) 6.58-6.75 (m, 1 H) 7.04-7.23 (m, 2H) 7.38-7.53 (m, 5H) 7.58-7.66 (m, 1H) 7.86 (brt, J=6.78 Hz, 1H) 7.92-8.00 (m, 2H) 8.02-8.15 (m, 2H) 8.35 (t, J=8.03 Hz, 1H) 8.78 (br d, J=5.77 Hz, 1H) LCMS: m/z 671.3 (M+H⁺).

### Example S104: Synthesis of (2R,3S,4aR,7aR)-2-[4-(cyclopentylamino)phenyl]-1-(2-fluoro-6-methyl-benzo yl)-N-[1-(4-pyridylmethyl)indazol-5-yl]-2,3,4,4a,5,6,7,7a-octahydrocyclopenta-[b]pyridine-3-carboxamide (Compound No. 87)

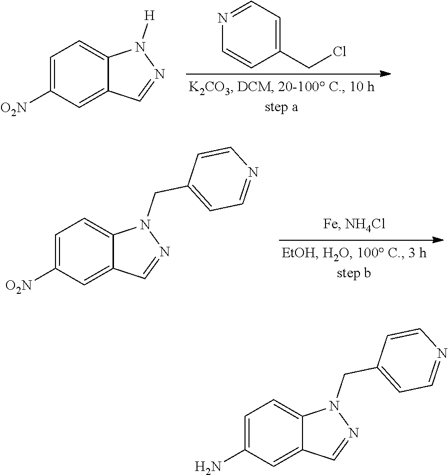 [SMILES: O=[N+]([O-])c1ccc2[nH]ncc2c1 ; ClCc1ccncc1 ; O=[N+]([O-])c1ccc2c(cnn2Cc2ccncc2)c1 ; Nc1ccc2c(cnn2Cc2ccncc2)c1]

Step a) To a mixture of 5-nitro-1H-indazole (2 g, 12.26 mmol) and 4-(chloromethyl) pyridine (2.41 g, 14.71 mmol, HCl) in DMF (10 mL) was added K₂CO₃ (5.08 g, 36.78 mmol) at 20° C. under N₂. The mixture was stirred at 100° C. for 5 h. The reaction mixture was partitioned between EtOAc 100 mL and H₂O 100 mL. The organic phase was separated, dried, filtered and concentrated under reduced pressure to give a residue. The residue was purified by Prep-HPLC: column: Welch Xtimate C18 150*40 mm*10 μm; mobile phase: [water (10 mM NH₄HCO₃)-ACN]; B %: 31%-51%, 8 min. 5-nitro-1-(pyridin-4-yl)-1H-indazole (300 mg, 1.18 mmol, 9.62% yield) was obtained as a yellow solid. ¹H NMR (400 MHz, CDCl₃) δ ppm 5.67 (s, 2H) 7.04 (d, J=6.02 Hz, 2H) 7.38 (d, J=9.29 Hz, 1H) 8.25-8.33 (m, 2H) 8.55-8.61 (m, 2H) 8.79 (d, J=2.01 Hz, 1H) LCMS: (ES) m/z 255.4 (M+H⁺).

Step b) A mixture of 5-nitro-1-(pyridin-4-yl)-1H-indazole (300 mg, 1.18 mmol), NH₄Cl (31.56 mg, 589.99 mol) and Fe (527.16 mg, 9.44 mmol) in EtOH (10 mL) and H₂O (2.5 mL) was degassed and purged with N₂ for 3 times, and then the mixture was stirred at 90° C. for 3 h under N₂ atmosphere. Filtered, then concentrated to get the desired product. Compound 1-(pyridin-4-yl)-1H-indazol-5-amine (220 mg, 981.00 mol, 83.14% yield) was obtained as a yellow solid. ¹H NMR (400 MHz, CDCl₃) δ ppm 5.54 (s, 2H) 6.84 (dd, J=8.78, 2.01 Hz, 1H) 6.97 (d, J=1.51 Hz, 1H) 6.99 (d, J=5.77 Hz, 2H) 7.10 (d, J=8.78 Hz, 1H) 7.88 (d, J=0.75 Hz, 1H) 8.51 (d, J=5.77 Hz, 2H) LCMS: (ES) m/z 225.4 (M+H⁺).

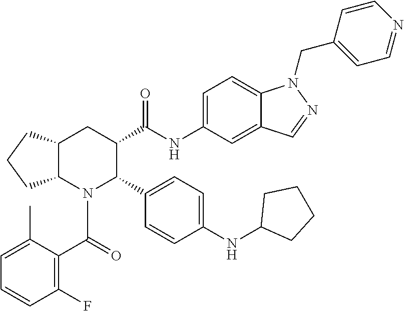 [SMILES: Cc1cccc(F)c1C(=O)N1[C@@H]2CCC[C@@H]2C[C@H](C(=O)Nc2ccc3c(cnn3Cc3ccncc3)c2)[C@@H]1c1ccc(NC2CCCC2)cc1]

The title compound was synthesized in similar fashion as Example S95. ¹H NMR (400 MHz, METHANOL-d₄) δ ppm 1.33 (br d, J=18.57 Hz, 5H) 1.50 (s, 1H) 1.61 (br d, J=4.77 Hz, 3H) 1.68-1.77 (m, 3H) 1.96-2.06 (m, 3H) 2.10-2.26 (m, 3H) 2.29 (s, 1H) 2.37-2.48 (m, 2H) 3.10 (br s, 1H) 3.66-3.81 (m, 2H) 5.72 (d, J=4.02 Hz, 2H) 6.57-6.68 (m, 3H) 7.05-7.17 (m, 4H) 7.34-7.51 (m, 5H) 7.85-8.00 (m, 1H) 8.07 (d, J=9.54 Hz, 1H) 8.45 (br s, 2H) LCMS: (ES) m/z 671.4 (M+H⁺).

### Example S105: Synthesis of cis-1-(2-fluoro-6-methylbenzoyl)-N-(4-methyl-3-(trifluoromethyl)-phenyl)-2-(4-((tetrahydro-2H-pyran-4-yl)amino)phenyl)octahydro-1H-cyclopenta[b]pyridine-3-carboxamide (Compound No. 88)

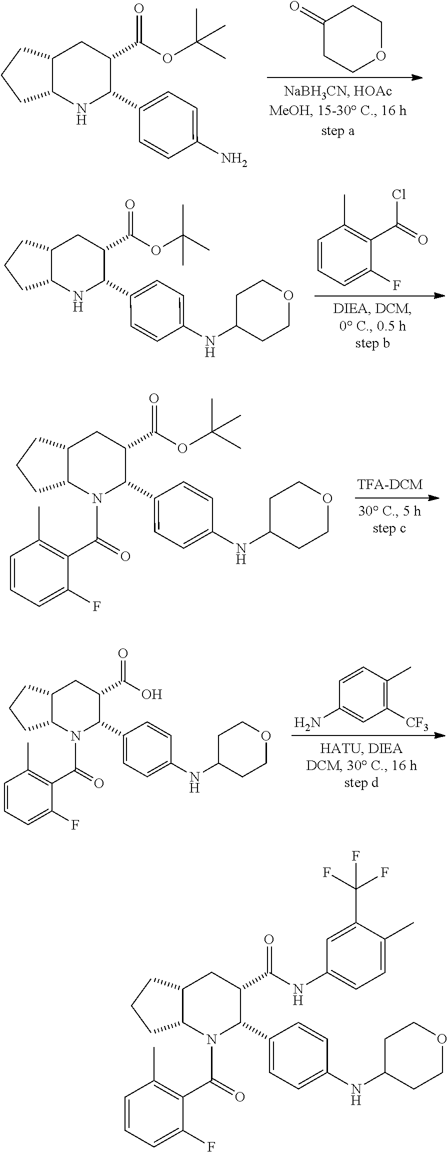

Step a) To a mixture of cis-tert-butyl2-(4-aminophenyl)-2,3,4,4a,5,6,7,7a-octahydro-1H-cyclopenta[b]pyridine-3-carboxylate (0.1 g, 316.02 mol) in MeOH (1.5 mL) was added tetrahydropyran-4-one (34.80 mg, 347.62 mol, 31.93 μL), HOAc (37.95 mg, 632.04 mol, 36.15 μL) and NaBH₃CN (119.15 mg, 1.90 mmol) in one portion at 15° C. The mixture was stirred at 30° C. for 16 h. The mixture was diluted with DCM (15 mL) and alkalified to pH=8-9 and extracted with DCM (3×10 mL). The combined organic layers were washed with brine, dried, filtered and concentrated in vacuo to give the crude. The crude was purified by prep-HPLC (column: Waters Xbridge Prep OBD C18 150×40 mm×m; mobile phase: [water (10 mM NH₄HCO₃)-ACN]; B %: 20%-90%, 20 min) to give cis-tert-butyl 2-[4-(tetrahy dropyran-4-ylamino)phenyl]-2,3,4,4a,5,6,7,7a-octahydro-1H-cyclopenta[b]pyridine-3-carboxylate (80 mg, 199.72 mol, 68.38% yield) as colorless gum. LC-MS: (ES) m/z 401.3 (M+H⁺).

Step b) To a solution of cis-tert-butyl2-[4-(tetrahydropyran-4-ylamino)phenyl]-2,3,4,4a,5,6,7,7a-octahydro-1H-cyclopenta[b]pyridine-3-carboxylate (80 mg, 199.72 mol) and DIEA (51.62 mg, 399.45 mol, 69.58 μL) in DCM (3 mL) was added dropwise of a solution of 2-fluoro-6-methyl-benzoyl chloride (34.47 mg, 199.72 mol) in DCM (2 mL) at 0° C. The mixture was stirred at 0° C. for 10 min. The mixture was diluted with DCM (20 mL), washed with H₂O (2×10 mL), dried, filtered and concentrated in vacuo to give the crude product. The crude product was purified by flash silica gel chromatography (ISCO®; 4 g SepaFlash® Silica Flash Column, eluent of 0˜50% ethyl acetate/petroleum ether gradient@22 mL/min) to give cis-tert-butyl 1-(2-fluoro-6-methyl-benzoyl)-2-[4-(tetrahydropyran-4-ylamino)phenyl]-2,3,4,4a,5,6,7,7a-octahydrocyclopenta-[b]pyridine-3-carboxylate (110 mg, 192.67 mol, 96.47% yield, 94% purity) as white solid. LC-MS: (ES) m/z 537.3 (M+H⁺).

Step c) The cis-tert-butyl1-(2-fluoro-6-methyl-benzoyl)-2-[4-(tetrahydropyran-4-yl-amino)phenyl]-2,3,4,4a,5,6,7,7a-octahydrocyclopenta[b]pyridine-3-carboxylate (98 mg, 182.61 mol) was dissolved in DCM (5 mL). Then CF₃COOH (1.54 g, 13.51 mmol, 1 mL) was added. The mixture was stirred at 15° C. for 16 h. Then 10 mL of H₂O was added. Then the mixture was extracted with EtOAc (15 mL×2). The combined organic layers were washed with brine (10 mL), dried over anhydrous Na₂SO₄, filtered and the filtrate was concentrated under reduced pressure to give cis-1-(2-fluoro-6-methyl-benzoyl)-2-[4-(tetrahydropyran-4-yl amino) phenyl]-2,3,4,4a,5,6,7,7a-octahydrocyclopenta[b]pyridine-3-carboxylic acid (85 mg, 168.03 mol, 92.02% yield, 95% purity) as white solid. LC-MS: (ES) m/z 481.2 (M+H⁺).

Step d) The HATU (18.99 mg, 49.94 mol) and DIEA (13.45 mg, 104.04 μmol, 18.12 μL) were added to a mixture of cis-1-(2-fluoro-6-methyl-benzoyl)-2-[4-(tetrahydropyran-4-ylamino)phenyl]-2,3,4,4a,5,6,7,7a-octahydrocyclopenta[b]pyridine-3-carboxylic acid (20 mg, 41.62 mol) and 4-methyl-3-(trifluoromethyl)aniline (8.75 mg, 49.94 mol, 7.17 μL) in DCM (0.5 mL). Then the mixture was stirred at 30° C. for 16 h. The reaction mixture was evaporated under vacuum to give the crude product. The crude product was purified by prep-HPLC (column: Venusil ASB Phenyl 150×30 mm×5 μm; mobile phase: [water (0.05% HCl)-ACN]; B %: 60%-90%, 9 min) to give cis-1-(2-fluoro-6-methyl-benzoyl)-N-[4-methyl-3-(trifluoromethyl)phenyl]-2-[4-(tetrahydropyran-4-ylamino)phenyl]-2,3,4,4a,5,6,7,7a-octahydrocyclopenta[b]pyridine-3-carboxamide (6 mg, 8.45 mol, 20.32% yield, 95% purity, HCl) as white solid. ¹H NMR (400 MHz, METHANOL-d₄) δ 1.18-1.40 (3H, m), 1.41-1.91 (8H, m), 1.94-2.12 (2H, m), 2.13-2.30 (2H, m), 2.32-2.48 (6H, m), 3.09-3.22 (1H, m), 3.38 (2 H, td, J=11.92, 1.83 Hz), 3.62-3.82 (1H, m), 3.87-4.02 (2 H, m), 6.47-6.64 (1H, m), 6.98-7.17 (2H, m), 7.21-7.42 (4H, m), 7.46-7.58 (1H, m), 7.72-7.84 (2H, m), 7.85-7.99 (1H, m). LC-MS: (ES) m/z 638.3 (M+H⁺).

### Example S106: Synthesis of (2R,3S,4aR,7aR)-1-(2-fluoro-6-methylbenzoyl)-N-(1-methyl-1H-indazol-5-yl)-2-(4-((tetrahydro-2H-pyran-4-yl)amino)phenyl)octahydro-1H-cyclopenta[b]-pyridine-3-carboxamide (Compound No. 89)

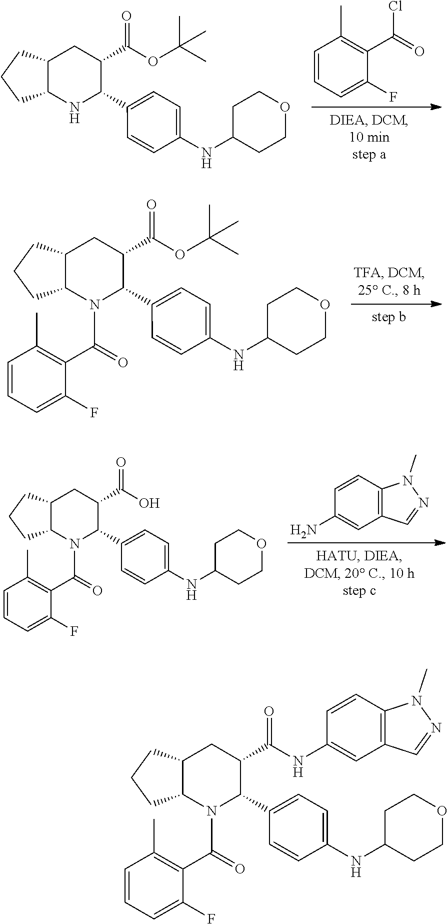

Step a) To a solution of (2R,3S,4aR,7aR)-tert-butyl 2-(4-((tetrahydro-2H-pyran-4-yl)amino)phenyl)octahydro-1H-cyclopenta[b]pyridine-3-carboxylate (800 mg, 2.00 mmol) and DIEA (516.26 mg, 3.99 mmol, 695.77 μL) in DCM (15 mL) was added 2-fluoro-6-methyl-benzoyl chloride (327.46 mg, 1.90 mmol) at 0° C., then the reaction mixture was stirred at 0° C. for 10 min. The reaction mixture was quenched by addition MeOH (5 mL) at 25° C., then the mixture was concentrated under reduced pressure to give a residue. The residue was purified by column chromatography (SiO₂, petroleum ether/ethyl acetate=100/1 to 0/1). The compound (2R,3 S,4aR,7aR)-tert-butyl 1-(2-fluoro-6-methylbenzoyl)-2-(4-((tetrahydro-2H-pyran-4-yl)amino)-phenyl)octahydro-1H-cyclopenta[b]pyridine-3-carboxylate (1.06 g, 1.84 mmol, 91.97% yield, 93% purity) was obtained as white solid. ¹H NMR (400 MHz, CDCl₃) δ ppm 1.15-1.24 (m, 3H), 1.33 (s, 4H), 1.39 (s, 5H), 1.40-1.54 (m, 5H), 1.94-2.09 (m, 6H), 2.33 (d, J=7.63 Hz, 3H), 3.47-3.57 (m, 4H), 3.98-4.03 (m, 2H), 6.50-6.62 (m, 3H), 6.91-6.96 (m, 1H), 7.01 (dd, J=7.57, 3.19 Hz, 1H), 7.19-7.25 (m, 1H), 7.29 (s, 1H), 7.34 (d, J=8.63 Hz, 1H). LC-MS: (ES) m/z 537.3 (M+H⁺).

Step b) To a solution of (2R,3S,4aR,7aR)-tert-butyl 1-(2-fluoro-6-methylbenzoyl)-2-(4-((tetrah ydro-2H-pyran-4-yl)amino)phenyl)octahydro-1H-cyclopenta[b]pyridine-3-carboxylate (1.06 g, 1.98 mmol) in DCM (5 mL) was added TFA (7.70 g, 67.53 mmol, 5 mL), then the reaction mixture was stirred at 25° C. for 8 h. The reaction mixture was concentrated under reduced pressure to give a residue. The residue was added HCl/dioxane at 25° C. for 10 min. Then the residue was concentrated under reduced pressure to give a crude product. The compound (2R,3S,4aR,7aR)-1-(2-fluoro-6-methylbenzoyl)-2-(4-((tetrahydro-2H-pyran-4-yl)amino)phenyl) octahydro-1H-cyclopenta[b]pyridine-3-carboxylic acid (930 mg, 1.74 mmol, 88.34% yield, 97% purity, HCl) was obtained as yellow solid. ¹H NMR (400 MHz, DMSO-d₆) δ ppm 0.97-1.14 (m, 2 H), 1.20-1.41 (m, 2H), 1.44-1.62 (m, 3H), 1.69-1.87 (m, 3H), 1.93-2.13 (m, 2H), 2.20-2.33 (m, 3H), 2.80-3.02 (m, 1H), 3.27-3.40 (m, 2H), 3.52-3.56 (m, 2H), 3.59-3.70 (m, 1H), 3.88 (br d, J=11.51 Hz, 2H), 6.42-6.48 (m, 1H), 6.92-7.22 (m, 5H), 7.29-7.41 (m, 3H). LC-MS: (ES) m/z 481.2 (M+H⁺).

Step c) To a solution of (2R,3S,4aR,7aR)-1-(2-fluoro-6-methylbenzoyl)-2-(4-((tetrahydro-2H-pyran-4-yl)amino)phenyl)octahydro-1H-cyclopenta[b]pyridine-3-carboxylic acid, HATU (47.47 mg, 124.85 mol) and DIEA (40.34 mg, 312.13 mol, 54.37 μL) in DCM (3 mL) at 25° C. for 10 min, then the 1-methylindazol-5-amine (22.97 mg, 156.06 mol) was added, then the reaction mixture was stirred at 25° C. for 2 h. The reaction mixture was concentrated under reduced pressure to give a crude product. The crude product was purified by prep-HPLC. (column: Phenomenex Gemini-NX 150*30 mm*5 μm; mobile phase: [water (0.05% HCl)-ACN]; B %: 30%-60%, 7 min). The compound (2R,3S,4aR,7aR)-1-(2-fluoro-6-methylbenzoyl)-N-(1-methyl-1H-indazol-5-yl)-2-(4-((tetrahydro-2H-pyran-4-yl)amino)phenyl)octahydro-1H-cyclopenta[b]pyridine-3-carboxamide (17 mg, 25.52 mol, 15.56% yield, 97% purity, HCl) was obtained as light yellow solid. ¹H NMR (400 MHz, METHANOL-d₄) δ ppm 1.21-1.33 (m, 2H), 1.55 (br t, J=10.51 Hz, 1H), 1.66-1.92 (m, 6 H), 2.02-2.13 (m, 2H), 2.15-2.35 (m, 2H), 2.42 (s, 2H), 3.18-3.30 (m, 2H), 3.33-3.41 (m, 2H), 3.67-3.84 (m, 2H), 3.98 (br d, J=11.88 Hz, 2H), 4.03-4.07 (m, 3H), 6.54-6.72 (m, 1H), 7.06 (t, J=8.69 Hz, 1H), 7.14-7.21 (m, 1H), 7.33-7.54 (m, 5H), 7.84-7.99 (m, 4H). LC-MS: (ES) m/z 610.3 (M+H⁺).

### Example S107: Synthesis of cis-1-(2-fluoro-6-methylbenzoyl)-N-(quinolin-7-yl)-2-(4-((tetrahydro-2H-pyran-4-yl)amino)phenyl)octahydro-1H-cyclopenta[b]pyridine-3-carboxamide (Compound No. 90)

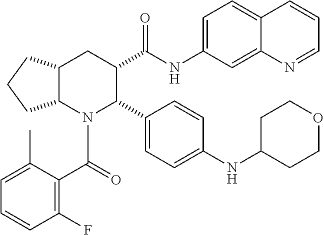

The title compound was synthesized in similar fashion as Example S106. ¹H NMR (400 MHz, METHANOL-d₄) δ 1.17-1.46 (3H, m), 1.47-1.93 (8H, m), 2.07-2.37 (1H, m), 2.10-2.22 (1H, m), 2.23-2.38 (2H, m), 2.45 (2 H, s), 3.36-3.47 (2H, m), 3.67-3.89 (2H, m), 3.92-4.04 (2H, m), 6.67-6.91 (1H, m), 7.02-7.14 (1H, m), 7.15-7.24 (1H, m), 7.33-7.46 (3H, m), 7.85-8.02 (4H, m), 8.25-8.34 (1H, m), 8.88-9.15 (3H, m). LC-MS: (ES) m/z 607.4 (M+H⁺).

### Example S108: Synthesis of (2S,3R,4aS,7aS)-1-(2-fluoro-6-methylbenzoyl)-N-(1-methyl-1H-indazol-5-yl)-2-(4-((tetrahydro-2H-pyran-4-yl)amino)phenyl)octahydro-1H-cyclopenta[b]-pyridine-3-carboxamide (Compound No. 91)

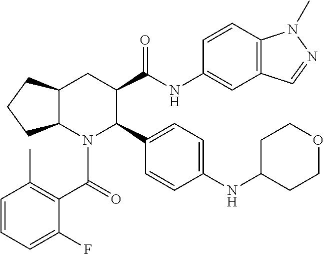 [SMILES: Cc1cccc(F)c1C(=O)N1[C@H](c2ccc(NC3CCOCC3)cc2)[C@H](C(=O)Nc2ccc3c(cnn3C)c2)C[C@@H]2CCC[C@@H]21]

Step a) To a solution of tert-butyl 2-(4-nitrophenyl)-6,7-dihydro-5H-cyclopenta[b]-pyridine-3-carboxylate (1.2 g, 3.53 mmol), HCl/dioxane (4 M, 1.76 mL) in MeOH (25 mL) was added PtO₂ (160.11 mg, 705.11 mol) under N₂. The suspension was degassed under vacuum and purged with H₂ several times. The mixture was stirred under H₂ (15 psi) at 20° C. for 3 h. The previous batch (4 g) was combined with this batch, then concentrated under reduced pressure to remove solvent. The residue was diluted with sat. NaHCO₃(aq) 100 ml and extracted with EtOAc (100 mL*3). The combined organic layers were dried, filtered and concentrated under reduced pressure to give a residue. The residue was purified by column chromatography (SiO₂, DCM:methanol=1/0 to 10/1) to get tert-butyl 2-(4-aminophenyl)-2,3,4,4a, 5,6,7,7a-octahydro-1H-cyclopenta[b]pyridine-3-carboxylate (2.4 g) as a light yellow gum. ¹H NMR (400 MHz, CDCl₃) δ ppm 1.17 (s, 9H), 1.45-1.63 (m, 3 H), 1.75-1.91 (m, 3H), 1.99-2.20 (m, 3H), 2.78 (q, J=6.05 Hz, 1H), 3.29 (td, J=6.54, 2.81 Hz, 1H), 3.55 (br s, 2H), 3.90 (d, J=5.63 Hz, 1H), 6.62 (d, J=8.38 Hz, 2H), 7.14 (d, J=8.38 Hz, 2H). LC-MS: (ES) m/z 317.2 (M+H⁺).

Step b) To a mixture of tert-butyl 2-(4-aminophenyl)-2,3,4,4a,5,6,7,7a-octahydro-1H-cyclopent a[b]pyridine-3-carboxylate (1.1 g, 3.48 mmol) and tetrahydropyran-4-one (417.63 mg, 4.17 mmol, 383.14 μL) in MeOH (15 mL) was added NaBH₃CN (655.36 mg, 10.43 mmol) at 0° C. under N₂. The mixture was stirred at 20° C. for 6 h, then NaBH₃CN (436.90 mg, 6.95 mmol) and AcOH (313.13 mg, 5.21 mmol, 298.22 μL) were added to the mixture, and stirred at 20° C. for another 6 h. The previous batch (1.5 g) was combined with this batch, and the mixture was concentrated under reduced pressure to remove solvent. The residue was diluted with saturated NaHCO₃ (200 ml) and extracted with EtOAc (200 mL*2). The combined organic layers were dried, filtered and concentrated under reduced pressure to give the tert-butyl 2-[4-(tetrahydropyran-4-ylamino)phenyl]-2,3,4,4a,5,6,7,7a-octahydro-1H-cyclopenta[b]pyridine-3-carboxylate (5 g, crude) as a light yellow oil. The crude was purified by column chromatography (SiO₂, DCM:methanol=1/0 to 10/1) to get a crude product (3.6 g, as a light yellow oil), then diluted with 1 M HCl(aq) 100 mL and washed with EtOAc (100 mL*2). The liquid layer was added sat.NaHCO₃(aq) (200 ml), then extracted with EtOAc (100 mL*3), the combined organic layers were dried, filtered, and concentrated under reduced pressure to give tert-butyl 2-[4-(tetrahydropyran-4-ylamino)phenyl]-2,3,4,4a,5,6,7,7a-octahydro-1H-cyclopenta[b] pyridine-3-carboxylate (2.0 g, 4.99 mmol, 40.00% yield). ¹H NMR (400 MHz, CDCl₃) δ ppm 1.12-1.24 (m, 9H), 1.39-1.59 (m, 5H), 1.69-1.92 (m, 4H), 2.00-2.06 (m, 3H), 2.10-2.18 (m, 1H), 2.76-2.83 (m, 1H), 3.30 (td, J=6.57, 2.87 Hz, 1H), 3.44-3.56 (m, 3H), 3.89-3.96 (m, 1H), 3.97-4.05 (m, 1H), 3.97-4.05 (m, 1H), 6.50-6.61 (m, 2H), 7.16 (d, J=8.44 Hz, 2H). LC-MS: (ES) m/z 401.3 (M+H⁺).

Step c) The tert-butyl 2-[4-(tetrahydropyran-4-ylamino)phenyl]-2,3,4,4a,5,6,7,7a-octahydro-1H-cyclopenta[b]pyridine-3-carboxylate (2 g, 4.99 mmol) was purified by Prep-SFC: column: Phenomenex-Cellulose-2 (250 mm*50 mm, 10 m); mobile phase: [0.1% NH₃·H₂O EtOH]; B %: 45%-45%, 8 min to give tert-butyl (2S,3R,4aS,7aS)-2-[4-(tetrahydropyran-4-ylamino)phenyl]-2,3, 4,4a,5,6,7,7a-octahydro-1H-cyclopenta[b]pyridine-3-carboxylate (850 mg, 2.12 mmol, 42.46% yield) (800 mg, 2.00 mmol, 40.00% yield, 100% ee) as a white solid (¹H NMR (400 MHz, CDCl₃) δ ppm 1.16 (s, 9H), 1.35-1.46 (m, 2H), 1.67-1.90 (m, 6H), 1.98-2.14 (m, 5H), 2.73-2.82 (m, 1H), 3.28 (td, J=6.65, 3.01 Hz, 1 H), 3.42-3.54 (m, 3H), 3.89 (d, J=6.02 Hz, 1H), 3.95-4.04 (m, 2H), 6.55 (d, J=8.53 Hz, 2 H), 7.14 (d, J=8.28 Hz, 2H). LC-MS: (ES) m/z 401.3 (M+H⁺)) and tert-butyl (2R,3S,4aR,7aR)-2-[4-(tetrahydropyran-4-ylamino)phenyl]-2,3,4,4a,5,6,7,7a-octahydro-1H-cyclopenta[b]pyridine-3-carboxylate (800 mg, 2.00 mmol, 40.06% yield, 99% ee) as a white solid (¹H NMR (400 MHz, CDCl₃) δ ppm 1.16 (s, 9H), 1.51-1.58 (m, 2H), 1.67-1.90 (m, 6H), 1.97-2.19 (m, 5H), 2.72-2.82 (m, 1H), 3.29 (td, J=6.65, 2.76 Hz, 1H), 3.41-3.55 (m, 3H), 3.90 (d, J=5.77 Hz, 1H), 3.95-4.04 (m, 2H), 6.55 (d, J=8.53 Hz, 2H), 7.15 (d, J=8.28 Hz, 2H). LC-MS: (ES) m/z 401.3 (M+H⁺)).

Step d) To a mixture of tert-butyl (2S,3R,4aS,7aS)-2-[4-(tetrahydropyran-4-ylamino)-phenyl]-2,3,4,4a,5,6,7,7a-octahydro-1H-cyclopenta[b]pyridine-3-carboxylate (50.00 mg, 124.83 mol) and DIEA (32.27 mg, 249.65 mol, 43.48 μL) in DCM (3 mL) was added 2-fluoro-6-methyl-benzoyl chloride (20.47 mg, 118.59 mol) at 0° C. under N₂. The mixture was stirred at 0° C. for 10 min. The reaction mixture was concentrated to get a residue. The residue was purified by prep-TLC (SiO₂, DCM:methanol=20:1). Tert-butyl (2S,3R,4aS,7aS)-1-(2-fluoro-6-methyl-benzoyl)-2-[4-(tetrahydropyran-4-ylamino)phenyl]-2,3,4,4a,5,6,7,7a-octahydrocyclopenta[b]pyridine-3-carboxylate (65 mg, 121.12 umol, 97.03% yield) was obtained as a colorless oil. LC-MS: (ES) m/z 537.3 (M+H⁺).

Step e) To a mixture of tert-butyl (2S,3R,4aS,7aS)-1-(2-fluoro-6-methyl-benzoyl)-2-[4-(tetrahydropyran-4-ylamino)phenyl]-2,3,4,4a,5,6,7,7a-octahydrocyclopenta[b]pyridine-3-carboxylate (65.00 mg, 121.12 mol) in DCM (5 mL) was added TFA (3.20 g, 28.10 mmol, 2.08 mL) at 25° C. under N₂. The mixture was stirred at 25° C. for 2.5 h. the mixture was concentrated to get a residue, then 4 M HCl/dioxane (10 mL) was added, and stirred at 25° C. for 10 min, then concentrated to get the desired product. Compound (2S,3R,4aS,7aS)-1-(2-fluoro-6-methyl-benzoyl)-2-[4-(tetrahydropyran-4-ylamino)phenyl]-2,3,4,4a,5,6,7,7a-octahydrocyclopenta[b]pyridine-3-carboxylic acid (60 mg, 116.05 mol, 95.82% yield, HCl) was obtained as a light yellow oil. LC-MS: (ES) m/z 481.2 (M+H⁺).

Step f) To a mixture of (2S,3R,4aS,7aS)-1-(2-fluoro-6-methyl-benzoyl)-2-[4-(tetrahydropyran-4-ylamino)phenyl]-2,3,4,4a,5,6,7,7a-octahydrocyclopenta[b]pyridine-3-carboxylic acid (60 mg, 116.05 mol, HCl) in DCM (3 mL) was added DIEA (44.99 mg, 348.14 mol, 60.64 μL) and HATU (52.95 mg, 139.26 mol) at 20° C. under N₂. The mixture was stirred at 20° C. for 10 min, then 1-methylindazol-5-amine (25.62 mg, 174.07 mol) was added and the mixture was stirred at 20° C. for 10 hr. The mixture was concentrated to get a residue. The residue was purified by Prep-HPLC: column: Phenomenex Gemini-NX 150*30 mm*5 μm; mobile phase: [water (0.05% HCl)-ACN]; B %: 18%-58%, 10 min.

(2S,3R,4aS,7aS)-1-(2-fluoro-6-meth yl-benzoyl)-N-(1-methylindazol-5-yl)-2-[4-(tetrahydropyran-4-ylamino)phenyl]-2,3,4,4a,5,6,7,7a-octahydrocyclopenta[b]pyridine-3-carboxamide (20 mg, 31.82 mol, 27.42% yield, 97% purity) was obtained as a light yellow solid. ¹H NMR (400 MHz, METHANOL-d₄) δ ppm 1.32-1.50 (m, 2H), 1.58-1.65 (m, 1 H), 1.67-1.95 (m, 6H), 2.03-2.17 (m, 2H), 2.17-2.31 (m, 2H), 2.34-2.51 (m, 2H), 3.22-3.32 (m, 2H), 3.35-3.45 (m, 2H), 3.70-3.87 (m, 2H), 4.00 (br d, J=11.88 Hz, 2H), 4.03-4.09 (m, 3H), 6.56-6.77 (m, 1H), 7.03-7.14 (m, 1H), 7.17-7.28 (m, 1H), 7.35-7.55 (m, 5H), 7.83-8.02 (m, 4H). LC-MS: (ES) m/z 610.3 (M+H⁺).

### Example S109: Synthesis of (2R,3S,4aR,7aR)-1-(2-fluoro-6-methyl-benzoyl)-N-[1-(2-pyridylmethyl)indazol-5-yl]-2-[4-(tetrahydropyran-4-ylamino)phenyl]-2,3,4,4a,5,6,7,7a-octahydrocyclopenta[b]pyridine-3-carboxamide (Compound No. 92)

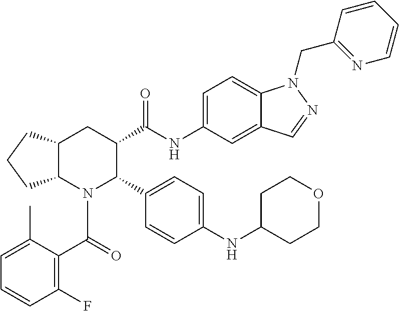 [SMILES: Cc1cccc(F)c1C(=O)N1[C@@H]2CCC[C@@H]2C[C@H](C(=O)Nc2ccc3c(cnn3Cc3ccccn3)c2)[C@@H]1c1ccc(NC2CCOCC2)cc1]

The title compound was synthesized in similar fashion as Example S108. ¹H NMR (400 MHz, METHANOL-d₄) δ ppm 1.26-1.36 (m, 2H) 1.45-1.65 (m, 2H) 1.66-1.90 (m, 6H) 2.00-2.17 (m, 2H) 2.18-2.33 (m, 2H) 2.43 (s, 2H) 3.25 (br dd, J=10.49, 5.13 Hz, 1 H) 3.37-3.53 (m, 2H) 3.75-3.84 (m, 1H) 3.99 (brd, J=9.30 Hz, 2H) 5.93-6.08 (m, 2H) 6.56-6.77 (m, 1H) 7.03-7.12 (m, 1H) 7.14-7.22 (m, 1H) 7.36-7.72 (m, 6H) 7.89-8.01 (m, 3H) 8.02-8.20 (m, 2H) 8.36-8.46 (m, 1H) 8.82 (br d, J=5.96 Hz, 1H) LC-MS: (ES) m/z 687.3 (M+H⁺).

### Example S110: Synthesis of (2R,3S,4aR,7aR)—N-[1-[1-(chloromethyl)-2-hydroxyethyl]indazol-5-yl]-1-(2-flu oro-6-methyl-benzoyl)-2-[4-(tetrahydropyran-4-ylamino)phenyl]-2,3,4,4a,5,6,7,7a-octahydrocyclopenta[b]pyridine-3-carboxamide (Compound No. 93)

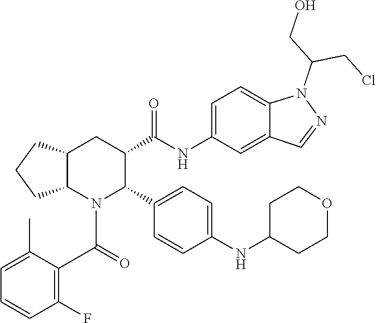

The title compound was synthesized in similar fashion as Example S108. ¹H NMR (400 MHz, METHANOL-d₄) δ ppm 1.31 (br s, 4H) 1.70 (br d, J=11.44 Hz, 2H) 1.75-1.85 (m, 3H) 1.96 (s, 1H) 2.04-2.15 (m, 2H) 2.20 (s, 1H) 2.26 (br d, J=9.06 Hz, 1H) 2.43 (s, 2H) 3.15 (s, 2H) 3.69-3.88 (m, 2H) 3.97-4.12 (m, 7H) 6.54-6.71 (m, 1H) 7.03-7.23 (m, 3H) 7.36-7.45 (m, 4H) 7.53-7.60 (m, 1H) 7.92-7.96 (m, 2H) 8.00-8.05 (m, 1H) LC-MS: (ES) m/z 688.3 (M+H⁺).

### Example S111: Synthesis of (2R,3S,4aR,7aR)—N-(4-(dimethylamino)phenyl)-1-(2-fluoro-6-methylbenzoyl)-2-(4-((tetrahydro-2H-pyran-4-yl)amino)phenyl)octahydro-1H-cyclopenta[b]pyridine-3-carboxamide (Compound No. 94)

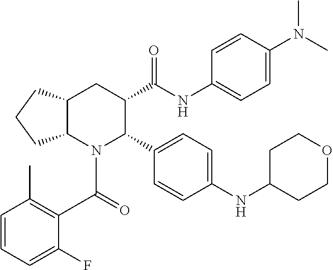 [SMILES: Cc1cccc(F)c1C(=O)N1[C@@H]2CCC[C@@H]2C[C@H](C(=O)Nc2ccc(N(C)C)cc2)[C@@H]1c1ccc(NC2CCOCC2)cc1]

The title compound was synthesized in similar fashion as Example S108. ¹H NMR (400 MHz, METHANOL-d₄) δ ppm 1.22-1.34 (m, 2H), 1.54 (br d, J=12.96 Hz, 1H), 1.68-1.91 (m, 6H), 2.01-2.15 (m, 2H), 2.18-2.31 (m, 2H), 2.41 (s, 2H), 3.31 (dt, J=3.27, 1.60 Hz, 8H), 3.37-3.46 (m, 2H), 3.73-4.05 (m, 4H), 6.53-6.70 (m, 1H), 7.01-7.20 (m, 2H), 7.37-7.48 (m, 3H), 7.52-7.65 (m, 2H), 7.69-7.82 (m, 2H), 7.84-7.94 (m, 2H). LC-MS: (ES) m/z 599.3 (M+H⁺).

### Example S112: Synthesis of (2R,3S,4aR,7aR)-1-(2-fluoro-6-methylbenzoyl)-N-(quinolin-6-yl)-2-(4-((tetrahyd ro-2H-pyran-4-yl)amino)phenyl)octahydro-JH-cyclopenta[b]pyridine-3-carboxamide (Compound No. 95)

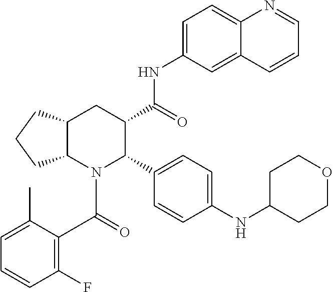 [SMILES: Cc1cccc(F)c1C(=O)N1[C@@H]2CCC[C@@H]2C[C@H](C(=O)Nc2ccc3ncccc3c2)[C@@H]1c1ccc(NC2CCOCC2)cc1]

The title compound was synthesized in similar fashion as Example S108. ¹H NMR (400 MHz, METHANOL-d₄) δ ppm 1.33 (br s, 3H), 1.62-1.86 (m, 6H), 2.08-2.31 (m, 4 H), 2.45 (s, 2H), 3.21-3.27 (m, 1H), 3.36-3.55 (m, 2H), 3.74-4.14 (m, 4H), 6.59-6.87 (m, 1H), 7.03-7.25 (m, 2H), 7.34-7.59 (m, 3H), 7.84-8.12 (m, 3H), 8.18-8.36 (m, 2 H), 8.65-8.89 (m, 1H), 8.96-9.21 (m, 2H). LC-MS: (ES) m/z 607.3 (M+H⁺).

### Example S113: (2R,3S,4aR,7aR)-1-(2-fluoro-6-methylbenzoyl)-N-(JH-indazol-5-yl)-2-(4-((tetrahydro-2H-pyran-4-yl)amino)phenyl)octahydro-1H-cyclopenta[b]pyridine-3-carboxamide (Compound No. 96)

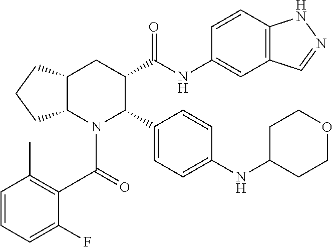 [SMILES: Cc1cccc(F)c1C(=O)N1[C@@H]2CCC[C@@H]2C[C@H](C(=O)Nc2ccc3[nH]ncc3c2)[C@@H]1c1ccc(NC2CCOCC2)cc1]

The title compound was synthesized in similar fashion as Example S108. ¹H NMR (400 MHz, METHANOL-d₄) δ ppm 1.25-1.37 (m, 2H), 1.56 (br d, J=13.63 Hz, 1H), 1.66-1.87 (m, 6H), 2.05-2.16 (m, 2H), 2.17-2.30 (m, 2H), 2.42 (s, 2H), 3.24 (br d, J=5.00 Hz, 2H), 3.37 (br t, J=11.44 Hz, 2H), 3.74-4.04 (m, 4H), 6.55-6.72 (m, 1H), 7.04-7.23 (m, 2H), 7.36-7.58 (m, 5H), 7.88-7.98 (m, 2H), 8.02-8.13 (m, 1H), 8.22-8.30 (m, 1 H). LC-MS: (ES) m/z 596.3 (M+H⁺).

### Example S114: (2R,3S,4aR,7aR)-1-(2-fluoro-6-methylbenzoyl)-N-(2-methyl-2H-indazol-5-yl)-2-(4-((tetrahydro-2H-pyran-4-yl)amino)phenyl)octahydro-1H-cyclopenta[b]pyridine-3-carboxamide (Compound No. 97)

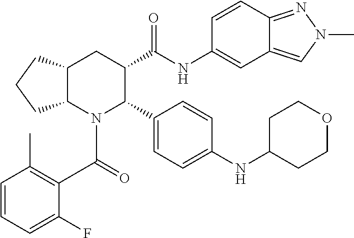

The title compound was synthesized in similar fashion as Example S108. ¹H NMR (400 MHz, METHANOL-d₄) δ ppm 1.28 (br d, J=7.50 Hz, 2H), 1.43-1.64 (m, 2H), 1.66-1.91 (m, 6H), 2.04-2.16 (m, 2H), 2.18-2.31 (m, 2H), 2.42 (s, 2H), 3.18-3.28 (m, 1H), 3.34-3.41 (m, 2H), 3.72-4.04 (m, 4H), 4.21-4.30 (m, 3H), 6.55-6.71 (m, 1H), 7.03-7.20 (m, 2H), 7.35-7.49 (m, 4H), 7.52-7.61 (m, 1H), 7.87-7.96 (m, 2H), 7.97-8.13 (m, 1H), 8.30-8.40 (m, 1H). LC-MS: (ES) m/z 610.3 (M+H⁺).

### Example S115: (2R,3S,4aR,7aR)-1-(2-fluoro-6-methylbenzoyl)-N-(1-methyl-1H-indol-5-yl)-2-(4-((tetrahydro-2H-pyran-4-yl)amino)phenyl)octahydro-1H-cyclopenta[b]pyridine-3-carboxamide (Compound No. 98)

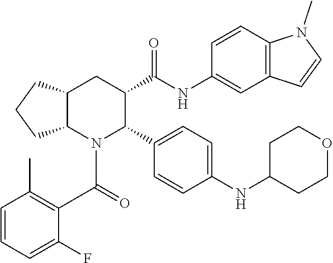 [SMILES: Cc1cccc(F)c1C(=O)N1[C@@H]2CCC[C@@H]2C[C@H](C(=O)Nc2ccc3c(ccn3C)c2)[C@@H]1c1ccc(NC2CCOCC2)cc1]

The title compound was synthesized in similar fashion as Example S108. ¹H NMR (400 MHz, METHANOL-d₄) δ ppm 1.31 (br d, J=13.01 Hz, 2H), 1.56-1.88 (m, 8H), 2.04-2.14 (m, 2H), 2.15-2.26 (m, 2H), 2.39-2.44 (m, 2H), 3.16-3.26 (m, 1H), 3.36-3.42 (m, 2H), 3.67-3.84 (m, 5H), 3.95 (br s, 2H), 6.51-6.68 (m, 1H), 7.03-7.19 (m, 4H), 7.24-7.45 (m, 5H), 7.53-7.78 (m, 1H), 7.82-7.95 (m, 2H). LC-MS: (ES) m/z 609.3 (M+H⁺).

### Example S116: (2R,3S,4aR,7aR)-1-(2-fluoro-6-methylbenzoyl)-N-(1-(oxetan-3-yl)-1H-indazol-5-yl)-2-(4-((tetrahydro-2H-pyran-4-yl)amino)phenyl)octahydro-1H-cyclopenta[b]pyridine-3-carboxamide (Compound No. 99)

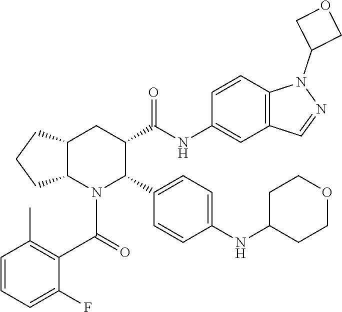 [SMILES: Cc1cccc(F)c1C(=O)N1[C@@H]2CCC[C@@H]2C[C@H](C(=O)Nc2ccc3c(cnn3C3COC3)c2)[C@@H]1c1ccc(NC2CCOCC2)cc1]

The title compound was synthesized in similar fashion as Example S108. ¹H NMR (400 MHz, METHANOL-d₄) δ ppm 1.28 (br d, J=4.05 Hz, 2H), 1.37-1.58 (m, 6H), 1.70 (br s, 1H), 1.95 (br d, J=11.44 Hz, 2H), 2.12-2.31 (m, 3H), 2.35-2.45 (m, 2H), 3.09 (br s, 1H), 3.40-3.57 (m, 3H), 3.75-4.00 (m, 3H), 4.59 (br s, 1H), 5.10-5.21 (m, 4H), 5.85-6.01 (m, 1H), 6.55-6.66 (m, 3H), 6.97-7.19 (m, 2H), 7.35-7.53 (m, 4H), 7.82-8.13 (m, 2H). LC-MS: (ES) m/z 652.3 (M+H⁺).

### Example S117: (2R,3S,4aR,7aR)-1-(2-fluoro-6-methylbenzoyl)-N-(1-(1-methylpiperidin-4-yl)-1H-indazol-5-yl)-2-(4-((tetrahydro-2H-pyran-4-yl)amino)phenyl)octahydro-1H-cyclopenta[b]-pyridine-3-carboxamide (Compound No. 100)

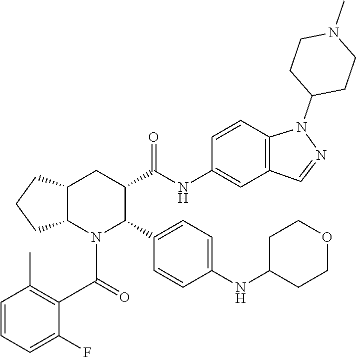

The title compound was synthesized in similar fashion as Example S108. ¹H NMR (400 MHz, METHANOL-d₄) δ ppm 1.23-1.41 (m, 3H), 1.55-1.85 (m, 7H), 2.04-2.15 (m, 2H), 2.26 (br d, J=13.83 Hz, 3H), 2.42 (s, 3H), 2.46-2.56 (m, 2H), 2.95-3.00 (m, 3 H), 3.23 (br dd, J=10.19, 5.66 Hz, 1H), 3.32-3.42 (m, 4H), 3.51-3.90 (m, 5H), 3.95 (br s, 2H), 6.56-6.77 (m, 1H), 6.97-7.26 (m, 3H), 7.27-7.39 (m, 1H), 7.45 (d, J=8.58 Hz, 2 H), 7.58-7.66 (m, 1H), 7.87-8.03 (m, 4H). LC-MS: (ES) m/z 693.4 (M+H⁺).

### Example S118: (2R,3S,4aR,7aR)-1-(2-fluoro-6-methylbenzoyl)-N-(1-(pyridin-4-ylmethyl)-1H-indazol-5-yl)-2-(4-((tetrahydro-2H-pyran-4-yl)amino)phenyl)octahydro-1H-cyclopenta[b]-pyridine-3-carboxamide (Compound No. 101)

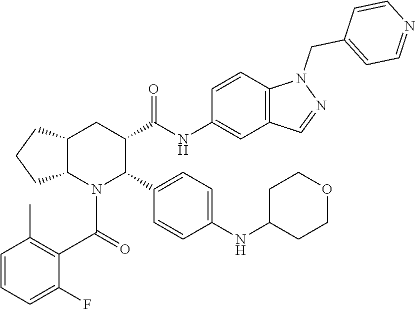 [SMILES: Cc1cccc(F)c1C(=O)N1[C@@H]2CCC[C@@H]2C[C@H](C(=O)Nc2ccc3c(cnn3Cc3ccncc3)c2)[C@@H]1c1ccc(NC2CCOCC2)cc1]

The title compound was synthesized in similar fashion as Example S108. ¹H NMR (400 MHz, METHANOL-d₄) δ ppm 1.21-1.39 (m, 3H), 1.48-1.90 (m, 8H), 2.00-2.13 (m, 2H), 2.14-2.27 (m, 2H), 2.27-2.63 (m, 3H), 3.23 (dt, J=10.73, 5.36 Hz, 1H), 3.33-3.43 (m, 2H), 3.68-4.02 (m, 4H), 5.95-6.07 (m, 2H), 6.57-6.79 (m, 1H), 7.02-7.19 (m, 2H), 7.36-7.54 (m, 4H), 7.69-7.76 (m, 2H), 7.88-8.01 (m, 2H), 8.08 (br s, 1H), 8.11-8.18 (m, 1H), 8.75-8.79 (m, 2H). LC-MS: (ES) m/z 687.3 (M+H⁺).

### Example S119: (2R,3S,4aR,7aR)-1-(2-fluoro-6-methylbenzoyl)-N-(1-(2-hydroxyethyl)-1H-indazol-5-yl)-2-(4-((tetrahydro-2H-pyran-4-yl)amino)phenyl)octahydro-1H-cyclopenta[b]pyridine-3-carboxamide (Compound No. 102)

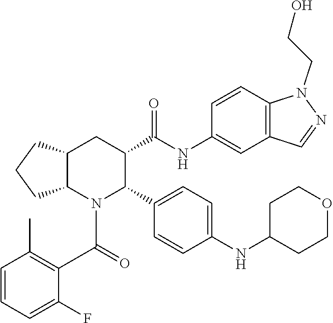 [SMILES: Cc1cccc(F)c1C(=O)N1[C@@H]2CCC[C@@H]2C[C@H](C(=O)Nc2ccc3c(cnn3CCO)c2)[C@@H]1c1ccc(NC2CCOCC2)cc1]

The title compound was synthesized in similar fashion as Example S108. ¹H NMR (400 MHz, METHANOL-d₄) δ ppm 1.26-1.48 (m, 2H), 1.53-1.62 (m, 1H), 1.69-1.94 (m, 6H), 2.03-2.15 (m, 2H), 2.18-2.33 (m, 2H), 2.44 (s, 2H), 3.24 (br d, J=9.66 Hz, 2H), 3.36-3.50 (m, 2H), 3.76-4.08 (m, 6H), 4.40-4.56 (m, 2H), 6.53-6.76 (m, 1H), 7.05-7.25 (m, 2H), 7.36-7.65 (m, 5H), 7.87-8.08 (m, 4H) LC-MS: (ES) m/z 640.3 (M+H⁺).

### Example S120: (2R,3S,4aR,7aR)—N-(1-(2-(dimethylamino)ethyl)-1H-indazol-5-yl)-1-(2-fluoro-6-methylbenzoyl)-2-(4-((tetrahydro-2H-pyran-4-yl)amino)phenyl)octahydro-1H-cyclopenta[b]-pyridine-3-carboxamide (Compound No. 103)

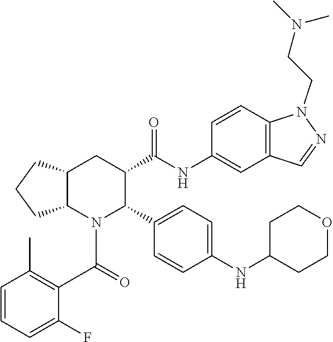 [SMILES: Cc1cccc(F)c1C(=O)N1[C@@H]2CCC[C@@H]2C[C@H](C(=O)Nc2ccc3c(cnn3CCN(C)C)c2)[C@@H]1c1ccc(NC2CCOCC2)cc1]

The title compound was synthesized in similar fashion as Example S108. ¹H NMR (400 MHz, METHANOL-d₄) δ ppm 1.35 (br s, 2H), 1.55 (br s, 1H), 1.66-1.97 (m, 6H), 2.13 (br d, J=9.66 Hz, 2H), 2.20-2.35 (m, 2H), 2.40-2.52 (m, 2H), 3.01 (s, 6H), 3.25 (br s, 2 H), 3.36-3.50 (m, 2H), 3.66-4.18 (m, 6H), 4.64-4.81 (m, 2H), 6.44-6.81 (m, 1H), 7.04-7.27 (m, 2H), 7.35-7.77 (m, 5H), 7.83-8.19 (m, 4H) LC-MS: (ES) m/z 667.4 (M+H⁺).

### Example S121: ((2R,3S,4aR,7aR)-1-(2-fluoro-6-methylbenzoyl)-N-(2-(2-hydroxyethyl)-2H-indaz ol-5-yl)-2-(4-((tetrahydro-2H-pyran-4-yl)amino)phenyl)octahydro-1H-cyclopenta[b]pyridine-3-carboxamide (Compound No. 104)

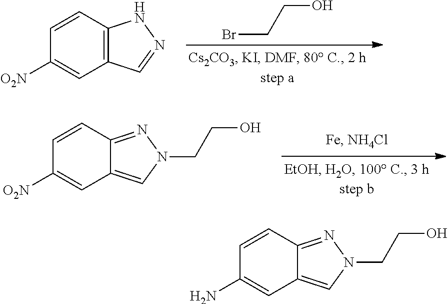

Step a) To a solution of 5-nitro-1H-indazole (1.4 g, 8.58 mmol) and 2-bromoethanol (1.39 g, 11.16 mmol, 792.14 μL) in DMF (15 mL) was added Cs₂CO₃ (5.59 g, 17.16 mmol) and KI (142.46 mg, 858.19 mol) at 20° C. under N₂. The mixture was stirred at 80° C. for 2 h. The reaction mixture was concentrated under reduced pressure to give a residue. The residue was purified by column chromatography (SiO₂, petroleum ether/ethyl acetate=100/1 to 0/1). The compound 2-(5-nitroindazol-2-yl)ethanol (520 mg, 2.51 mmol, 29.25% yield) was obtained as a yellow solid. ¹H NMR (400 MHz, CDCl₃) δ ppm 2.88 (br s, 1H), 4.19 (br s, 2H), 4.57-4.73 (m, 2H), 7.76 (d, J=9.38 Hz, 1H), 8.13 (dd, J=9.51, 2.13 Hz, 1H), 8.31 (s, 1H), 8.75 (d, J=2.13 Hz, 1H) LC-MS: (ES) m/z 208.1 (M+H⁺).

Step b) To a solution of 2-(5-nitroindazol-2-yl)ethanol (470 mg, 2.27 mmol) in EtOH (10 mL) and H₂O (2 mL) was added Fe (1.01 g, 18.15 mmol) and NH₄Cl (60.67 mg, 1.13 mmol). The mixture was stirred at 100° C. for 3 hr. The reaction mixture was filtered and the filtrate was concentrated under reduced pressure to give a residue. The residue was purified by column chromatography (SiO₂, DCM:methanol=100/1 to 10/1). The Compound 2-(5-aminoindazol-2-yl)ethanol (310 mg, 1.75 mmol, 77.12% yield, 100% purity) was obtained as a light yellow solid. ¹H NMR (400 MHz, CDCl₃) δ ppm 3.44-3.69 (m, 2H), 4.06-4.13 (m, 2H), 4.44-4.50 (m, 2H), 6.78 (d, J=1.51 Hz, 1H), 6.85 (dd, J=9.03, 2.01 Hz, 1H), 7.54 (d, J=9.03 Hz, 1H), 7.70 (s, 1H) LC-MS: (ES) m/z 178.1 (M+H⁺).

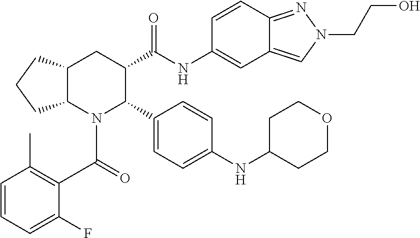 [SMILES: Cc1cccc(F)c1C(=O)N1[C@@H]2CCC[C@@H]2C[C@H](C(=O)Nc2ccc3nn(CCO)cc3c2)[C@@H]1c1ccc(NC2CCOCC2)cc1]

The title compound was synthesized in similar fashion as Example S108. ¹H NMR (400 MHz, METHANOL-d₄) δ ppm 1.24-1.33 (m, 2H), 1.56 (br d, J=15.77 Hz, 1H), 1.68-1.93 (m, 6H), 2.02-2.16 (m, 2H), 2.17-2.29 (m, 2H), 2.42 (s, 2H), 3.18-3.30 (m, 2H), 3.34-3.43 (m, 2H), 3.75-4.10 (m, 6H), 4.54-4.70 (m, 2H), 6.56-6.74 (m, 1H), 7.03-7.20 (m, 2H), 7.36-7.67 (m, 5H), 7.88-8.01 (m, 2H), 8.10-8.24 (m, 1H), 8.57 (br s, 1 H). LC-MS: (ES) m/z 640.3 (M+H⁺).

### Example S122: (2R,3S,4aR,7aR)—N-(2-(2-(dimethylamino)ethyl)-2H-indazol-5-yl)-1-(2-fluoro-6-methylbenzoyl)-2-(4-((tetrahydro-2H-pyran-4-yl)amino)phenyl)octahydro-1H-cyclopenta[b]-pyridine-3-carboxamide (Compound No. 105)

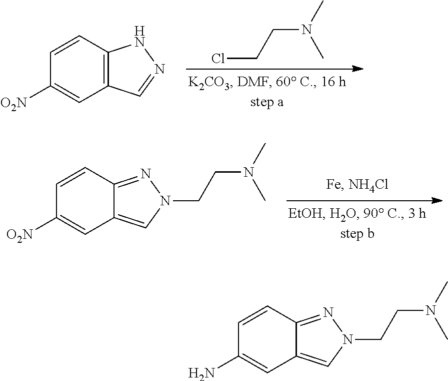

Step a) To a solution of 5-nitro-1H-indazole (1.65 g, 10.10 mmol) in DMF (20 mL) was added K₂CO₃ (4.33 g, 31.32 mmol), after 30 min, the 2-chloro-N,N-dimethyl-ethanamine (2.33 g, 16.16 mmol, HCl) was added. The mixture was stirred at 60° C. for 16 h showed the desired product was detected. The reaction mixture was diluted with H₂O 20 mL and extracted with EA 100 mL (50 mL*2). The combined organic layers were washed with brine 20 mL, dried over anhydrous Na₂SO₄, filtered and concentrated under reduced pressure to give a residue. The residue was purified by prep-HPLC. (column: Welch Xtimate C18 150*40 mm*10 m; mobile phase: [water (10 mM NH₄HCO₃)—CAN]; B %: 39%-47%, 7.2 min). The compound N,N-dimethyl-2-(5-nitroindazol-2-yl)ethanemine (0.42 g, 1.79 mmol, 17.72% yield, 100% purity) was obtained as a yellow solid. ¹H NMR (400 MHz, METHANOL-d₄) δ ppm 2.27-2.34 (m, 6H), 2.98 (t, J=6.40 Hz, 2H), 4.64 (t, J=6.53 Hz, 2 H), 7.73 (d, J=9.29 Hz, 1H), 8.10 (dd, J=9.54, 2.26 Hz, 1H), 8.66 (s, 1H), 8.81 (d, J=1.76 Hz, 1H). LC-MS: (ES) m/z 235.1 (M+H⁺).

Step b) To a solution of N,N-dimethyl-2-(5-nitroindazol-2-yl)ethanamine (0.4 g, 1.71 mmol), Fe (534 mg, 9.56 mmol) and NH₄Cl (32 mg, 598.23 μmol) in EtOH (10 mL) and H₂O (2.5 mL) was degassed and purged with N₂ 3 times, and then the mixture was stirred at 90° C. for 3 h under N₂ atmosphere. The reaction mixture was concentrated under reduced pressure to remove EtOH and H₂O. The residue was concentrated under reduced pressure to give a residue. The residue was purified by flash silica gel chromatography (ISCO®; 12 g SepaFlash® Silica Flash Column, Eluent of 0-10% ethylacetate/petroleum ether gradient@35 mL/min). The compound 2-[2-(dimethylamino)ethyl]indazol-5-amine (310 mg, 1.50 mmol, 99% purity) were obtained as a brown gum. ¹H NMR (400 MHz, CDCl₃) δ ppm 2.30 (s, 6H), 2.89 (t, J=6.75 Hz, 2H), 4.45 (t, J=6.82 Hz, 2H), 6.77 (d, J=2.00 Hz, 1H), 6.82 (dd, J=9.01, 2.13 Hz, 1H), 7.55 (d, J=9.13 Hz, 1H), 7.74 (s, 1H). LC-MS: (ES) m/z 205.1 (M+H⁺).

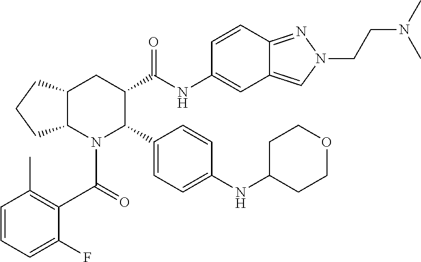

The title compound was synthesized in similar fashion as Example S108. ¹H NMR (400 MHz, METHANOL-d₄) δ ppm 1.26-1.36 (m, 2H), 1.53-1.59 (m, 1H), 1.70-1.90 (m, 6H), 2.04-2.14 (m, 2H), 2.18-2.32 (m, 2H), 2.42 (s, 2H), 2.98-3.05 (m, 6H), 3.14-3.29 (m, 2H), 3.33-3.42 (m, 2H), 3.76-4.04 (m, 6H), 4.74 (br s, 2H), 6.56-6.71 (m, 1 H), 6.99-7.18 (m, 2H), 7.23-7.58 (m, 5H), 7.89-8.04 (m, 3H), 8.24-8.36 (m, 1H). LC-MS: (ES) m/z 667.4 (M+H⁺).

### Example S123: (2R,3S,4aR,7aR)-1-(2-fluoro-6-methyl-benzoyl)-2-[4-(tetrahydropyran-4-ylamino)phenyl]-N-(1-tetrahydropyran-4-ylindazol-5-yl)-2,3,4,4a,5,6,7,7a-octahydro-cyclopenta[b]pyridine-3-carboxamide (Compound No. 106)

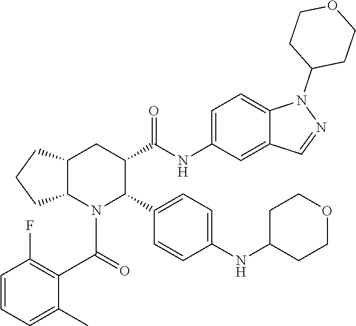 [SMILES: Cc1cccc(F)c1C(=O)N1[C@@H]2CCC[C@@H]2C[C@H](C(=O)Nc2ccc3c(cnn3C3CCOCC3)c2)[C@@H]1c1ccc(NC2CCOCC2)cc1]

The title compound was synthesized in similar fashion as Example S108. ¹H NMR (400 MHz, METHANOL-d₄) δ ppm 1.26-1.42 (m, 3H) 1.47-1.56 (m, 1H) 1.70 (br d, J=8.94 Hz, 2H) 1.75-1.85 (m, 3H) 1.94 (br d, J=12.99 Hz, 2H) 2.08-2.16 (m, 2H) 2.20 (s, 1H) 2.25-2.33 (m, 3H) 2.40-2.50 (m, 2H) 3.14-3.28 (m, 2H) 3.38 (br s, 2 H) 3.69 (br t, J=12.10 Hz, 3H) 3.79-3.89 (m, 1H) 3.96-4.15 (m, 5H) 6.56-6.70 (m, 1H) 7.01-7.23 (m, 3H) 7.36-7.45 (m, 4H) 7.58-7.64 (m, 1H) 7.88-7.98 (m, 4H) LC-MS: (ES) m z 680.3 (M+H⁺).

### Example S124: Synthesis of (2R,3S,4aR,7aR)-1-(2-fluoro-6-methyl-benzoyl)-N-[1-(3-pyridylmethyl)indazol-5-yl]-2-[4-(tetrahydropyran-4-ylamino)phenyl]-2,3,4,4a,5,6,7,7a-octahydrocyclopenta[b]pyridine-3-carboxamide (Compound No. 107)

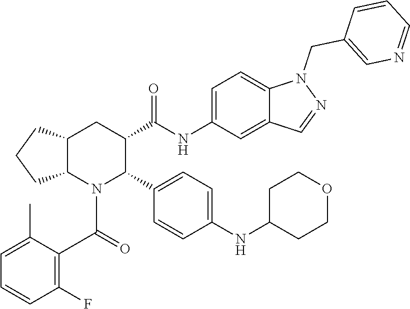 [SMILES: Cc1cccc(F)c1C(=O)N1[C@@H]2CCC[C@@H]2C[C@H](C(=O)Nc2ccc3c(cnn3Cc3cccnc3)c2)[C@@H]1c1ccc(NC2CCOCC2)cc1]

The title compound was synthesized in similar fashion as Example S108. ¹H NMR (400 MHz, METHANOL-d₄) δ ppm 1.24-1.37 (m, 3H) 1.57 (br d, J=17.64 Hz, 1H) 1.65-1.80 (m, 4H) 1.82-1.89 (m, 2H) 2.06-2.17 (m, 2H) 2.18-2.29 (m, 2H) 2.39-2.47 (m, 2 H) 3.20-3.27 (m, 1H) 3.35-3.46 (m, 2H) 3.69-3.90 (m, 2H) 3.97 (brs, 2H) 5.91 (brd, J=3.46 Hz, 2H) 6.57-6.72 (m, 1H) 6.58-6.72 (m, 1H) 7.01-7.11 (m, 1H) 7.14-7.21 (m, 1H) 7.37-7.52 (m, 4H) 7.61-7.70 (m, 1H) 7.91-7.98 (m, 2H) 7.99-8.08 (m, 2H) 8.08-8.14 (m, 1H) 8.38-8.45 (m, 1H) 8.75-8.83 (m, 2H). LC-MS: (ES) m/z 687.3 (M+H⁺).

### Example S125: Synthesis of (2R,3S,4aR,7aR)—N-[1-(cyclopropylmethyl)indazol-5-yl]-1-(2-fluoro-6-methyl-benzoyl)-2-[4-(tetrahydropyran-4-ylamino)phenyl]-2,3,4,4a,5,6,7,7a-octahydro-cyclopenta[b]pyridine-3-carboxamide (Compound No. 108)

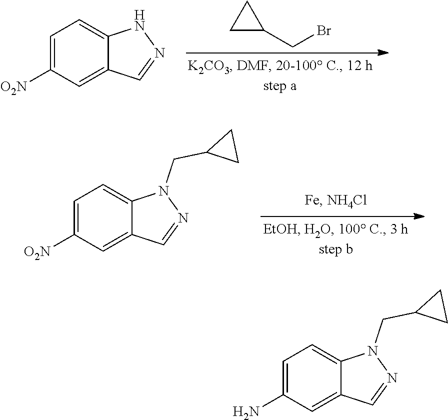 [SMILES: O=[N+]([O-])c1ccc2[nH]ncc2c1 ; BrCC1CC1 ; O=[N+]([O-])c1ccc2c(cnn2CC2CC2)c1 ; Nc1ccc2c(cnn2CC2CC2)c1]

Step a) To a mixture of bromomethylcyclopropane (1.99 g, 14.71 mmol, 1.41 mL) and 5-nitro-1H-indazole (2.00 g, 12.26 mmol) in DMF (10 mL) was added K₂CO₃ (5.08 g, 36.78 mmol) at 20° C. under N₂. The mixture was stirred at 100° C. for 12 h. The reaction mixture was partitioned between EtOAc 100 mL and H₂O 100 mL. The organic phase was separated, dried, filtered and concentrated under reduced pressure to give a residue. The residue was purified by Prep-HPLC (column: YMC-Triart Prep C18 150*40 mm*7 μm; mobile phase: [water (0.04% NH₃H₂O+10 mM NH₄HCO₃)-ACN]; B %: 40%-70%, 10 min) to give 1-(cyclopropy lmethyl)-5-nitro-indazole (1.3 g, 5.98 mmol, 48.81% yield) as a yellow solid. ¹H NMR (400 MHz, CDCl₃) δ ppm 0.36-0.51 (m, 2H) 0.54-0.69 (m, 2H) 1.29-1.44 (m, 1H) 4.32 (d, J=7.03 Hz, 2H) 7.49 (d, J=9.29 Hz, 1H) 8.21 (s, 1H) 8.27 (d, J=9.29 Hz, 1H) 8.73 (s, 1H) LCMS: (ES) m/z 218.3 (M+H⁺).

Step b) A mixture of 1-(cyclopropylmethyl)-5-nitro-indazole (500 mg, 2.30 mmol, 1 eq), Fe (1.03 g, 18.41 mmol, 8 eq) and NH₄Cl (61.56 mg, 1.15 mmol, 0.5 eq) in EtOH (10 mL) and H₂O (2.5 mL) was degassed and purged with N₂ for 3 times, and then the mixture was stirred at 90° C. for 3 h under N₂ atmosphere. Filtered, then concentrated to get the desired product. 1-(cyclopropyl-methyl)indazol-5-amine (400 mg, 2.14 mmol, 92.81% yield) was obtained as a yellow solid. ¹H NMR (400 MHz, CDCl₃) δ ppm 0.33-0.48 (m, 2H) 0.50-0.64 (m, 2H) 1.23-1.40 (m, 1H) 3.09 (br s, 3H) 4.22 (d, J=6.78 Hz, 2H) 6.90 (br d, J=8.53 Hz, 1H) 6.98 (s, 1H) 7.26-7.31 (m, 1H) 7.81 (s, 1H) LCMS: (ES) m/z 188.3 (M+H⁺).

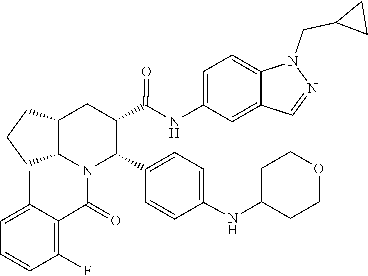 [SMILES: Cc1cccc(F)c1C(=O)N1[C@@H]2CCC[C@@H]2C[C@H](C(=O)Nc2ccc3c(cnn3CC3CC3)c2)[C@@H]1c1ccc(NC2CCOCC2)cc1]

The title compound was synthesized in similar fashion as Example S108. ¹H NMR (400 MHz, METHANOL-d₄) δ ppm 0.42 (br d, J=5.02 Hz, 2H) 0.56 (br d, J=8.28 Hz, 2H) 1.26-1.48 (m, 5H) 1.51-1.89 (m, 8H) 2.12 (brd, J=8.28 Hz, 2H) 2.20 (s, 1H) 2.28 (s, 1H) 2.44 (s, 2H) 3.13-3.30 (m, 1H) 3.77 (br d, J=7.03 Hz, 2H) 3.98 (br s, 3H) 4.26-4.33 (m, 2H) 6.54-6.72 (m, 1H) 7.00-7.32 (m, 3H) 7.37-7.48 (m, 4H) 7.52-7.58 (m, 1H) 7.90-7.99 (m, 4H) LCMS: (ES) m/z 687.3 (M+H⁺).

### Example S126: Synthesis of (2R,3S,4aR,7aR)—N-[1-(2-fluoroethyl)indazol-5-yl]-1-(2-fluoro-6-methyl-benzoyl)-2-[4-(tetrahydropyran-4-ylamino)phenyl]-2,3,4,4a,5,6,7,7a-octahydrocyclopenta-[b]pyridine-3-carboxamide (Compound No. 109)

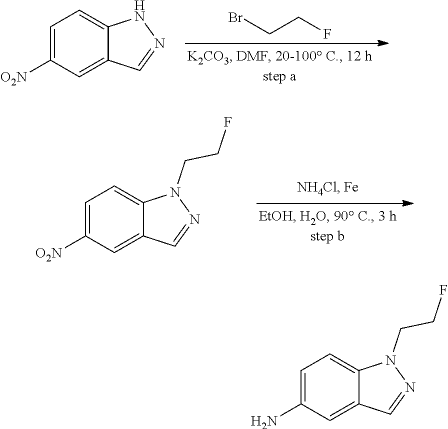

Step a) To a mixture of 1-bromo-2-fluoro-ethane (1.87 g, 14.71 mmol) and 5-nitro-1H-indazole (2.00 g, 12.26 mmol) in DMF (10 mL) was added K₂CO₃ (3.39 g, 24.52 mmol) at 20° C. under N₂. The mixture was stirred at 100° C. for 12 h. The reaction mixture was partitioned between EtOAc 100 mL and H₂O 100 mL. The organic phase was separated, dried, filtered and concentrated under reduced pressure to give a residue. The residue was purified by Prep-HPLC (column: Welch Xtimate C18 150*40 mm*10 m; mobile phase: [water (10 mM NH₄HCO₃)-ACN]; B %: 35%-55%, 8 min) to give 1-(2-fluoroethyl)-5-nitro-indazole (1.4 g, 6.69 mmol, 54.59% yield) as a yellow solid. ¹H NMR (400 MHz, CDCl₃) δ ppm 4.69-4.75 (m, 1H) 4.78 (t, J=4.64 Hz, 1H) 4.82-4.87 (m, 1H) 4.96 (t, J=4.64 Hz, 1H) 7.56 (d, J=9.29 Hz, 1H) 8.28 (d, J=0.75 Hz, 1H) 8.32 (dd, J=9.16, 2.13 Hz, 1H) 8.76 (d, J=1.51 Hz, 1H) LCMS: (ES) m/z 210.6 (M+H⁺).

Step b) A mixture of 1-(2-fluoroethyl)-5-nitro-indazole (400 mg, 1.91 mmol), NH₄Cl (51.14 mg, 956.13 mol) and Fe (854.32 mg, 15.30 mmol) in EtOH (10 mL) and H₂O (2.5 mL) was degassed and purged with N₂ for 3 times, and then the mixture was stirred at 90° C. for 3 h under N₂ atmosphere, filtered, then concentrated to get the desired product. 1-(2-fluoroethyl)indazol-5-amine (300 mg, 1.67 mmol, 87.55% yield) was obtained as a yellow solid. ¹H NMR (400 MHz, CDCl₃) δ ppm 4.58 (t, J=4.89 Hz, 1H) 4.64 (t, J=4.89 Hz, 1H) 4.77 (t, J=5.02 Hz, 1H) 4.88 (t, J=5.02 Hz, 1H) 6.86-6.96 (m, 2H) 7.29 (d, J=8.78 Hz, 1H) 7.83 (s, 1H) LCMS: (ES) m/z 180.1 (M+H⁺).

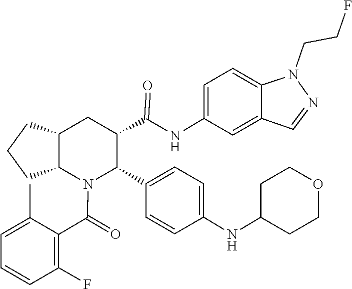

The title compound was synthesized in similar fashion as Example S108. ¹H NMR (400 MHz, METHANOL-d₄) δ ppm 1.30-1.48 (m, 3H) 1.52-1.65 (m, 2H) 1.67-1.77 (m, 3H) 1.78-1.88 (m, 3H) 2.06-2.17 (m, 2H) 2.20 (s, 1H) 2.24-2.37 (m, 1H) 2.43-2.48 (m, 2H) 3.22-3.28 (m, 1H) 3.35-3.42 (m, 2H) 3.72-4.01 (m, 4H) 4.62-4.79 (m, 4H) 6.56-6.70 (m, 1H) 7.01-7.11 (m, 1H) 7.14-7.21 (m, 1H) 7.37-7.46 (m, 4H) 7.51-7.58 (m, 1H) 7.92-8.02 (m, 4H) LCMS: (ES) m/z 643.3 (M+H⁺).

### Example S127: Synthesis of cis-2-(4-(cyclopentylamino)phenyl)-N-(4-methyl-3-(trifluoromethyl)-phenyl)-1-(oxazole-4-carbonyl)octahydro-1H-cyclopenta[b]pyridine-3-carboxamide (Compound No. 110)

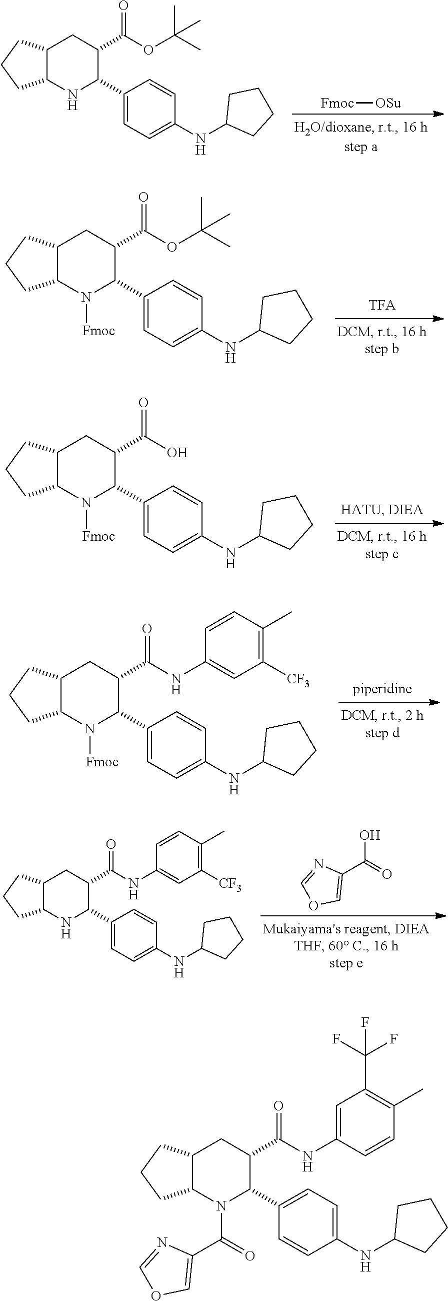

Step a) A solution of NaHCO₃ (290.54 mg, 3.46 mmol, 134.51 μL) in H₂O (20 mL) was added to a solution of cis-tert-butyl 2-[4-(cyclopentylamino)phenyl]-2,3,4,4a,5,6,7,7a-octahydro-1H-cyclopenta[b]pyridine-3-carboxylate (700 mg, 1.73 mmol) in dioxane (20 mL) and Fmoc-OSu (583.34 mg, 1.73 mmol) was added. The mixture was stirred at 15° C. for 16 h. The reaction mixture was extracted with EtOAc (30 mL×2). The combined organic phase were washed with brine, dried with anhydrous Na₂SO₄ and filtered. The filtrate was evaporated under vacuum to give a residue. The residue was purified by column chromatography (SiO₂, petroleum ether/ethyl acetate=100/0 to 3/1) to give compound cis-3-tert-butyl 1-(9H-fluoren-9-ylmethyl) 2-[4-(cyclopentylamino)phenyl]-2,3,4,4a,5,6,7,7a-octahydrocyclopenta[b]pyridine-1,3-dicarboxylate (1 g, 1.65 mmol, 95.30% yield, 100% purity) as a white solid. ¹H NMR (400 MHz, DMSO-d₆) δ 0.94 (1 H, br s) 1.23 (8 H, s) 1.40 (5H, dt, J=12.11, 5.87 Hz) 1.47-1.80 (8H, m) 1.87 (3H, br s) 2.68 (1 H, br s) 3.55-3.84 (2 H, m) 4.25-4.37 (1H, m) 4.43-4.68 (2H, m) 5.48 (2H, d, J=6.53 Hz) 6.36 (2H, br d, J=7.78 Hz) 6.77 (2H, br d, J=7.53 Hz) 7.23-7.47 (4H, m) 7.64 (2H, br d, J=7.03 Hz) 7.83-7.93 (2H, m). LC-MS: (ES) m/z 607.4 (M+H⁺).

Step b) The cis-3-tert-butyl1-(9H-fluoren-9-ylmethyl) 2-[4-(cyclopentylamino)phenyl]-2,3,4,4a, 5,6,7,7a-octahydrocyclopenta[b]pyridine-1,3-dicarboxylate (1 g, 1.65 mmol) was dissolved in DCM (20 mL). Then CF₃COOH (1.88 g, 16.48 mmol, 1.22 mL) was added. And the mixture was stirred at 15° C. for 2 h. The mixture was evaporated under vacuum to give crude product. The crude product was added H₂O (20 mL), extracted with EtOAc (30 mL×2). The combined organic phase was washed with brine, dried with anhydrous Na₂SO₄ and filtered. The filtrate was evaporated under vacuum to give compound cis-2-[4-(cyclopentylamino)phenyl]-1-(9H-fluoren-9-ylmethoxy-carbonyl)-2,3,4,4a,5,6,7,7a-octahydrocyclopenta[b]pyridine-3-carboxylic acid (900 mg, 1.63 mmol, 99.17% yield) as a white solid. ¹H NMR (400 MHz, CDCl₃) δ 0.78 (1 H, br s), 1.31-1.67 (6H, m), 1.68-2.12 (10H, m), 2.80 (1 H, br s), 3.62-4.05 (2H, m), 4.23 (1 H, br s), 4.47-4.80 (2H, m), 537-5.96 (1H, m), 6.99-7.20 (4H, m), 7.25-7.41 (4H, m), 7.53 (2 H, br d, J=7.34 Hz), 7.72 (2H, br d, J=7.34 Hz), 9.43 (1 H, br s). LC-MS: (ES) m/z 551.3 (M+H⁺).

Step c) The cis-2-[4-(cyclopentylamino)phenyl]-1-(9H-fluoren-9-ylmethoxycarbonyl)-2,3,4,4a,5,6,7,7a-octahydrocyclopenta[b]pyridine-3-carboxylic acid (0.9 g, 1.63 mmol) and 4-methyl-3-(trifluoromethyl)aniline (343.50 mg, 1.96 mmol, 281.56 μL) were dissolved in DCM (20 mL). Then DIEA (528.05 mg, 4.09 mmol, 711.66 uL, 2.5 eq) and HATU (745.70 mg, 1.96 mmol, 1.2 eq) were added. The mixture was stirred at 20° C. for 16 h. The reaction mixture was added to H₂O (20 mL) and was extracted with DCM (20 mL×2). The combined organic layers were washed with brine (20 mL), dried with anhydrous Na₂SO₄ and filtered. The filtrate was evaporated under vacuum to give a residue. The residue was purified by column chromatography (SiO₂, petroleum ether/ethyl acetate=100/0 to 2/1) to give compound cis-9H-fluoren-9-ylmethyl2-[4-(cyclopentyl-amino)phenyl]-3-[[4-methyl-3-(trifluoromethyl)phenyl]carbamoyl]-2,3,4,4a,5,6,7,7a-octahydro-cyclopenta[b]pyridine-1-carboxylate (1 g, 1.41 mmol, 86.44% yield, 100% purity) as a white solid. ¹H NMR (400 MHz, CDCl₃) δ 1.34-1.51 (5H, m), 1.53-1.75 (8H, m), 1.79-2.02 (5H, m), 2.41 (3 H, br s), 2.77 (1 H, br s), 3.51-3.77 (2H, m), 4.25 (2 H, br s) 4.50-4.70 (2H, m), 5.42-5.88 (1 H, m), 6.40 (2H, d, J=8.53 Hz), 6.88-7.10 (2H, m), 7.17 (1H, br d, J=8.03 Hz), 7.28-7.60 (8H, m), 7.73 (2H, br d, J=6.02 Hz). LC-MS: (ES) m/z 708.3 (M+H⁺).

Step d) The cis-9H-fluoren-9-ylmethyl2-[4-(cyclopentylamino)phenyl]-3-[[4-methyl-3-(trifluoromethyl)phenyl]carbamoyl]-2,3,4,4a,5,6,7,7a-octahydrocyclopenta[b]pyridine-1-carboxylate (1 g, 1.41 mmol) was dissolved in DCM (10 mL). Then piperidine (862.20 mg, 10.13 mmol, 1 mL) was added. The mixture was stirred at 20° C. for 2 h. Then another portion of piperidine (862.20 mg, 10.13 mmol, 1 mL) was added and the mixture was stirred at 20° C. for another 1 h. The reaction mixture was diluted with H₂O 10 mL and extracted with DCM (10 mL×2). The combined organic layers were washed with brine 10 mL, dried with anhydrous Na₂SO₄, filtered and concentrated under reduced pressure to give a residue. The residue was purified by column chromatography (SiO₂, DCM:methanol=100/0 to 100/1) to give cis-2-[4-(cyclopentylamino)phenyl]-N-[4-methyl-3-(trifluoromethyl)phenyl]-2,3,4,4a,5,6,7, 7a-octahydro-1H-cyclopenta[b]pyridine-3-carboxamide (550 mg, 1.09 mmol, 76.97% yield, 96% purity) as a pale yellow solid. ¹H NMR (400 MHz, CDCl₃) δ 1.32-1.45 (2H, m), 1.59-1.94 (1H, m), 1.94 (1 H, dt, J=12.65, 6.27 Hz), 2.04-2.16 (1H, m), 2.17-2.34 (2H, m), 2.38 (3H, d, J=1.22 Hz), 2.76-2.83 (1 H, m), 3.40 (1 H, br t, J=4.03 Hz), 3.58 (1 H, br s), 3.64-3.74 (1H, m), 3.89 (1H, d, J=2.69 Hz), 6.49 (2H, d, J=8.56 Hz), 7.06 (2H, d, J=8.56 Hz), 7.10-7.17 (1H, m), 7.55 (1H, dd, J=8.31, 1.71 Hz), 7.64 (1H, d, J=1.96 Hz) 11.17 (1 H, s). LC-MS: (ES) m/z 486.3 (M+H⁺).

Step e) The cis-2-[4-(cyclopentylamino)phenyl]-N-[4-methyl-3-(trifluoromethyl)-phenyl]-2,3,4, 4a,5,6,7,7a-octahydro-1H-cyclopenta[b]pyridine-3-carboxamide (30 mg, 61.78 mol) and oxazole-4-carboxylicacid (6.99 mg, 61.78 mol) were dissolved in THF (1 mL). Then 2-chloro-1-methyl-pyridin-1-ium iodide (23.68 mg, 92.67 mol) and DIEA (23.95 mg, 185.34 mol, 32.28 μL) were added. The mixture was stirred at 60° C. for 16 h. The solvent was evaporated under vacuum to give crude product. The crude product was purified by prep-HPLC (column: Venusil ASB Phenyl 150×30 mm×5 μm; mobile phase: [water (0.05% HCl)-ACN]; B %: 50%-80%, 9 min) to give compound cis-2-[4-(cyclopentylamino)phenyl]-N-[4-methyl-3-(trifluoromethyl)phenyl]-1-(oxazole-4-carbonyl)-2,3,4,4a,5,6,7,7a-octahydrocyclopenta[b]pyridine-3-carboxamide (13 mg, 18.81 mol, 30.44% yield, 84% purity) as a white solid. ¹H NMR (400 MHz, METHANOL-d₄) δ 1.29-1.45 (1H, m), 1.49-1.75 (9H, m), 1.77-2.15 (1H, m), 1.77-2.15 (8H, m), 2.30 (1 H, br s), 2.43 (4H, d, J=1.25 Hz), 3.09-3.25 (1H, m), 3.92 (1 H, br t, J=6.90 Hz), 4.76-4.88 (1H, m), 6.55 (1 H, br s), 7.22-7.33 (3H, m), 7.56 (1H, br d, J=8.28 Hz), 7.66 (2 H, br s), 7.86 (1H, d, J=2.26 Hz), 8.29 (1 H, s), 8.40 (1 H, br s), 10.12 (1 H, br s). LC-MS: (ES) m/z 581.3 (M+H⁺).

### Example S128: Synthesis of cis-2-(4-(cyclopentylamino)phenyl)-N-(4-methyl-3-(trifluoro-methyl)phenyl)-1-(tetrahydro-2H-pyran-4-carbonyl)octahydro-1H-cyclopenta[b]pyridine-3-carboxamide (Compound No. 111)

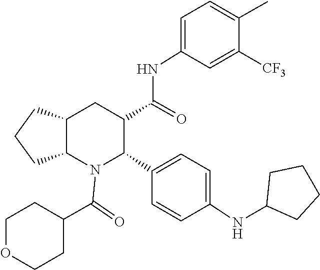

The title compound was synthesized in similar fashion as Example S127. ¹H NMR (400 MHz, METHANOL-d₄) δ 1.67 (br s, 5H), 1.81 (br d, J=12.55 Hz, 6H), 1.93-2.11 (m, 5H), 2.25-2.61 (m, 7H), 3.00-3.27 (m, 2H), 3.46-3.62 (m, 2H), 3.90 (br d, J=7.78 Hz, 1 H), 3.98 (br s, 2H), 4.17-4.49 (m, 1H), 4.51-4.80 (m, 1H), 6.25 (br d, J=5.77 Hz, 1H), 7.23-7.29 (m, 1H), 7.30-7.45 (m, 3H), 7.46-7.58 (m, 2H), 7.59-7.76 (m, 2H), 7.79-7.91 (m, 1H), 10.01-10.32 (m, 1H). LC-MS: (ES) m/z 598.4 (M+H⁺).

### Example S129: Synthesis of cis-2-(4-(cyclopentylamino)phenyl)-1-(1-methyl-1H-pyrazole-4-carbonyl)-N-(4-methyl-3-(trifluoromethyl)phenyl)octahydro-1H-cyclopenta[b]pyridine-3-carboxamide (Compound No. 112)

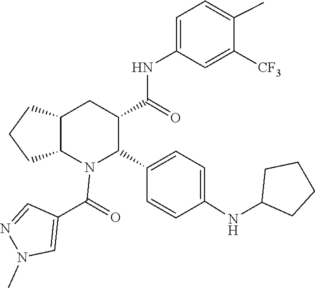

The title compound was synthesized in similar fashion as Example S127. ¹H NMR (400 MHz, METHANOL-d₄) δ 0.78 (2 H, br s), 1.28-1.33 (2H, m), 1.42-1.52 (5H, m), 1.67-1.78 (5H, m), 2.12-2.25 (2H, m), 2.31 (3H, d, J=1.22 Hz), 2.90-3.00 (1H, m), 3.60 (1 H, quin, J=6.24 Hz), 3.72 (3 H, s), 4.22-4.32 (1H, m), 4.50 (1 H, br s), 5.17-5.25 (1H, m), 6.46 (2H, d, J=8.56 Hz), 6.85 (2H, d, J=8.56 Hz), 7.18 (1H, d, J=8.31 Hz), 7.44-7.53 (1H, m), 7.57 (1 H, s), 7.72 (1H, d, J=1.71 Hz), 7.76 (1 H, s). LC-MS: (ES) m/z 594.3 (M+H⁺).

### Example S130: Synthesis of cis-2-(4-(cyclopentylamino)phenyl)-1-(1-methyl-1H-imidazole-4-carbonyl)-N-(4-methyl-3-(trifluoromethyl)phenyl)octahydro-1H-cyclopenta[b]pyridine-3-carboxamide (Compound No. 113)

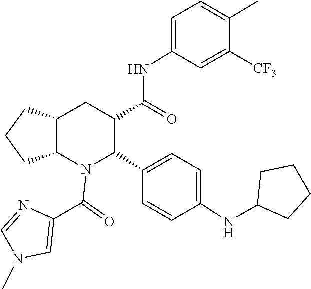

The title compound was synthesized in similar fashion as Example S127. ¹H NMR (400 MHz, METHANOL-d₄) δ 0.65-0.90 (6H, m), 1.19 (7 H, br s), 1.78-1.91 (4H, m), 1.96-2.15 (3H, m), 2.30 (3H, d, J=0.98 Hz), 2.98 (1 H, br s), 3.54-3.62 (1H, m), 3.66 (3H, s), 4.50 (1H, s), 4.56-4.71 (1H, m), 6.32-6.46 (3H, m), 7.04 (1 H, br s), 7.17 (1H, d, J=8.31 Hz), 7.38 (1 H, br s), 7.45 (1H, br d, J=8.31 Hz), 7.58 (1 H, br s) 7.71 (1 H, s). LC-MS: (ES) m/z 594.4 (M+H⁺).

### Example S131: Synthesis of cis-2-(4-(cyclopentylamino)phenyl)-N-(4-methyl-3-(trifluoromethyl)-phenyl)-1-(thiazole-4-carbonyl)octahydro-1H-cyclopenta[b]pyridine-3-carboxamide (Compound No. 114)

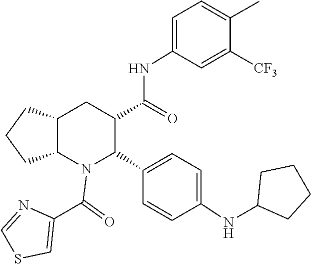

The title compound was synthesized in similar fashion as Example S127. ¹H NMR (400 MHz, METHANOL-d₄) δ 1.21-1.63 (13H, m), 1.76-2.20 (5H, m), 2.30 (3H, d, J=1.47 Hz), 2.99 (1 H, br s), 3.17-3.27 (2H, m), 3.60 (1 H, brt, J=6.11 Hz), 4.14 (1 H, br s), 6.41 (3 H, br s), 7.15 (3H, br d, J=8.31 Hz), 7.42 (1H, br d, J=7.34 Hz), 7.68 (1 H, br s), 8.95 (1H, d, J=1.96 Hz). LC-MS: (ES) m/z 597.3 (M+H⁺).

### Example S132: Synthesis of cis-2-(4-(cyclopentylamino)phenyl)-N-(4-methyl-3-(trifluoromethyl)-phenyl)-1-(pyrimidine-5-carbonyl)octahydro-1H-cyclopenta[b]pyridine-3-carboxamide (Compound No. 115)

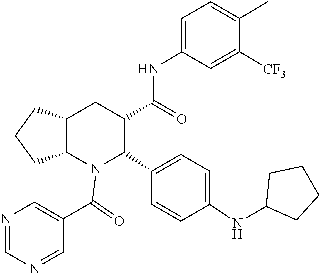

The title compound was synthesized in similar fashion as Example S127. ¹H NMR (400 MHz, METHANOL-d₄) δ 1.29-1.42 (1H, m), 1.44-1.64 (4H, m), 1.71 (7 H, br s), 1.86 (4 H, br s), 2.01 (4 H, br s), 2.42 (5 H, s), 3.19-3.27 (2H, m), 3.97 (1 H, br s), 7.30 (1 H, d, J=8.03 Hz), 7.43 (3H, br d, J=7.28 Hz), 7.55 (2H, br d, J=6.78 Hz), 7.61-7.96 (3H, m), 10.10 (1 H, br s). LC-MS: (ES) m/z 592.3 (M+H⁺).

### Example S133: Synthesis of (2R,3S,4aR,7aR)-1-(2-fluoro-6-methylbenzoyl)-N-(1-methyl-1H-indazol-5-yl)-2-(4-(((R)-2-(trifluoromethyl)pyrrolidin-1-yl)methyl)phenyl)octahydro-1H-cyclopenta[b]pyridine-3-carboxamide (Compound No. 116)

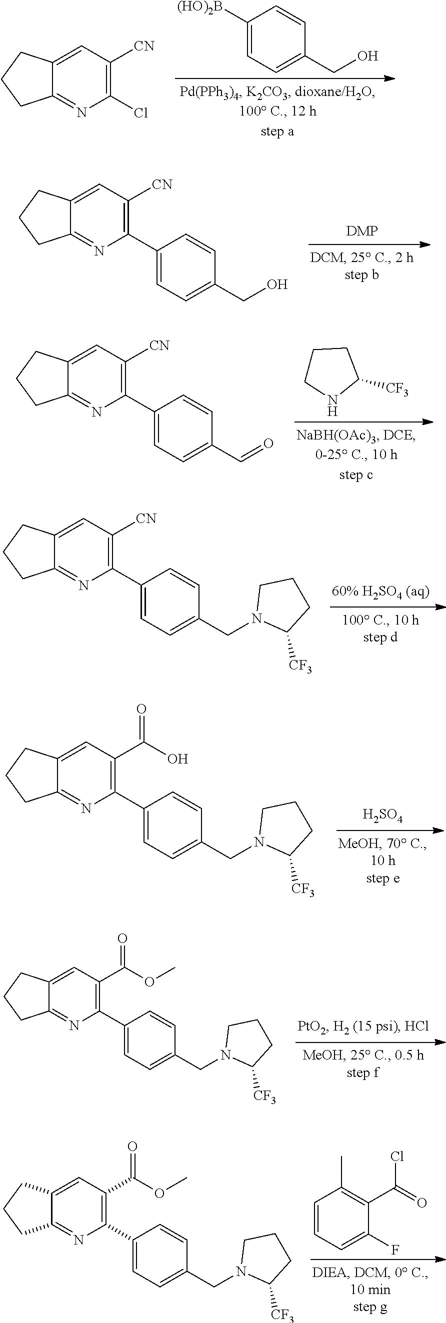

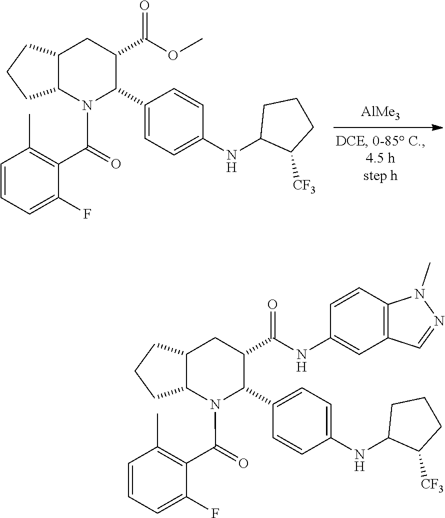

03421 Step a) To a solution of 2-chloro-6,7-dihydro-5H-cyclopenta[b]pyridine-3-carbonitrile (1 g, 5.60 mmol), [4-(hydroxymethyl)phenyl]boronic acid (1.11 g, 7.28 mmol), Pd(PPh₃)₄ (646.94 mg, 559.85 mol) and K₂CO₃ (2.32 g, 16.80 mmol) in dioxane (15 mL) and H₂O (15 mL), then the reaction mixture was stirred at 100° C. for 12 h. The reaction mixture was concentrated under reduced pressure to give a residue. The residue was filtered with EtOAc (100 ml) and filtrate was concentrated under reduced pressure to give a crude product. The crude product was added H₂O (20 mL) and acidized with HCl to pH=5, then extracted with DCM (50 mL*2). Then the aqueous phase was adjust to pH=8 with saturated Na₂CO₃ (aq) (50 ml) and extracted with DCM (50 mL*2). The combined organic layers were dried over Na₂SO₄, filtered and concentrated under reduced pressure to give the desired product. The compound 2-[4-(hydroxymethyl)phenyl]-6,7-dihydro-5H-cyclopenta[b]pyridine-3-carbonitrile (1.2 g, 4.70 mmol, 83.92% yield, 98% purity) was obtained as white solid. ¹H NMR (400 MHz, CDCl₃) δ ppm 2.14-2.31 (m, 3H), 3.04 (t, J=7.58 Hz, 2H), 3.14 (t, J=7.83 Hz, 2H), 4.76 (s, 2H), 7.48 (d, J=8.31 Hz, 2H), 7.77-7.91 (m, 3H). LC-MS: (ES) m/z 251.1 (M+H⁺).

Step b) To a mixture of 2-[4-(hydroxymethyl)phenyl]-6,7-dihydro-5H-cyclopenta[b]-pyridine-3-carbonitrile (600 mg, 2.40 mmol) in DCM (20 mL) was added DMP (1.53 g, 3.60 mmol, 1.11 mL) at 0° C. under N₂. The mixture was stirred at 25° C. for 2 h. The reaction mixture was quenched by addition Na₂S₂O₃(aq) 20 mL at 25° C., and then diluted with NaHCO₃(aq) 20 mL and extracted with DCM (20 mL*3). The combined organic layers were dried over Na₂SO₄, filtered and concentrated under reduced pressure to give the desired product. Compound 2-(4-formylphenyl)-6,7-dihydro-5H-cyclopenta[b]pyridine-3-carbonitrile (590 mg, 2.26 mmol, 94.18% yield, 95% purity) was obtained as a light yellow solid. LC-MS: (ES) m/z 249.1 (M+H⁺).

Step c) To a mixture of 2-(4-formylphenyl)-6,7-dihydro-5H-cyclopenta[b]pyridine-3-carbonitrile (380 mg, 1.53 mmol) and (2R)-2-(trifluoromethyl)pyrrolidine (425.85 mg, 3.06 mmol) in DCE (10 mL) was added NaBH(OAc)₃ (973.15 mg, 4.59 mmol) at 0° C. under N₂. The mixture was stirred at 25° C. for 10 h. The reaction mixture was quenched by addition sat.Na₂S₂O₃ solution 20 mL at 25° C., and then diluted with a saturated NaHCO₃ solution (20 mL) and extracted with DCM (20 mL*3). The combined organic layers were dried, filtered and concentrated under reduced pressure to give the residue. The residue was purified by column chromatography (SiO₂, DCM:methanol=100/1 to 5/1) and 2-[4-[[(2R)-2-(trifluoromethyl)pyrrolidin-1-yl]methyl]phenyl]-6,7-dihydro-5H-cyclopenta[b]pyridine-3-carbonitrile (300 mg, 807.76 umol, 52.78% yield) was obtained as a light yellow solid. ¹H NMR (400 MHz, METHANOL-d₄) δ ppm 0.85-0.92 (m, 1H) 1.27 (br s, 1H) 1.73-1.92 (m, 1H) 1.75-1.92 (m, 2H) 1.96-2.09 (m, 2H) 2.24 (quin, J=7.64 Hz, 2H) 2.35-2.46 (m, 1H) 3.00-3.06 (m, 2H) 3.11-3.21 (m, 2H) 3.26-3.34 (m, 1H) 3.69 (d, J=13.45 Hz, 1H) 4.26 (d, J=13.69 Hz, 1H) 7.48 (d, J=8.07 Hz, 2H) 7.67-7.95 (m, 3H). LC-MS: (ES) m/z 372.2 (M+H⁺).

Step d) To a solution of 2-[4-[[(2R)-2-(trifluoromethyl)pyrrolidin-1-yl]methyl]phenyl]-6,7-dihydro-5H-cyclopenta[b]pyridine-3-carbonitrile (280 mg, 753.91 mol) in H₂SO₄ (3 mL) and H₂O (3 mL), then the reaction mixture was stirred at 100° C. for 10 h. The reaction was adjusted to pH=4 with saturated NaHCO₃ solution and the reaction mixture was lyophilized. Then was added MeOH (20 mL), added filtered to get the filtrate. The filtrate was concentrated under reduced pressure to remove MeOH to give a crude product. Compound 2-[4-[[(2R)-2-(trifluoromethyl)-pyrrolidin-1-yl] methyl]phenyl]-6,7-dihydro-5H-cyclopenta[b]pyridine-3-carboxylic acid (500 mg, crude, contains Na₂SO₄) was obtained as a yellow solid. LC-MS: (ES) m/z 391.2 (M+H⁺).

Step e) To a solution of 2-[4-[[(2R)-2-(trifluoromethyl)pyrrolidin-1-yl]methyl]phenyl]-6,7-dihydro-5H-cyclopenta[b]pyridine-3-carboxylic acid (400 mg, 1.02 mmol) in MeOH (10 mL), Then the H₂SO₄ (20.51 mg, 204.92 mol, 11.15 μL, 98% purity) was added at 25° C., then the reaction mixture was stirred at 70° C. for 10 h. The reaction mixture was partitioned between EtOAc (100 mL) and saturated NaHCO₃(aq) (100 mL). The organic phase was separated, dried, filtered and concentrated under reduced pressure to give the crude. The compound methyl 2-[4-[[(2R)-2-(tri-fluoromethyl)pyrrolidin-1-yl]methyl]phenyl]-6,7-dihydro-5H-cyclopenta[b]pyridine-3-carboxylate (201 mg, crude) was obtained as a yellow oil. LC-MS: (ES) m/z 405.2 (M+H⁺).

Step f) To a solution of methyl 2-[4-[[(2R)-2-(trifluoromethyl)pyrrolidin-1-yl]methyl]phenyl]-6,7-dihydro-5H-cyclopenta[b]pyridine-3-carboxylate (180 mg, 445.08 mol) in MeOH (10 mL) was added HCl (4 M, 222.54 μL), then the PtO₂ (30.32 mg, 133.52 mol) was added. Then the reaction mixture was stirred at 25° C. for 0.5 h under H₂ (15 Psi). The reaction mixture was filtered and the filtrate was concentrated under reduced pressure to give the crude product. The crude product was purified by prep-TLC (SiO₂, DCM:MeOH=20:1). Compound methyl 2-[4-[[(2R)-2-(trifluoromethyl)pyrrolidin-1-yl]methyl]phenyl]-2,3,4,4a,5,6,7,7a-octahydro-1H-cyclopenta[b]pyridine-3-carboxylate (100 mg, crude) was obtained as a light yellow oil. LC-MS: (ES) m/z 411.2 (M+H⁺).

Step g) To a mixture of methyl 2-[4-[[(2R)-2-(trifluoromethyl)pyrrolidin-1-yl]methyl]phenyl]-2,3,4,4a,5,6,7,7a-octahydro-1H-cyclopenta[b]pyridine-3-carboxylate (99 mg, 241.19 mol) and 2-fluoro-6-methyl-benzoyl chloride (49.95 mg, 289.42 mol) in DCM (3 mL) was added DIEA (62.34 mg, 482.37 mol, 84.02 μL) at 0° C. under N₂. The mixture was stirred at 0° C. for 10 min. The reaction was concentrated to get a residue. The residue was purified by prep-TLC (SiO₂, DCM:MeOH=20:1). The compound methyl 1-(2-fluoro-6-methyl-benzoyl)-2-[4-[[(2R)-2-(trifluoromethyl)pyrrolidin-1-yl]methyl]phenyl]-2,3,4,4a,5,6,7,7a-octahydrocyclopenta[b]pyridine-3-carboxylate (130 mg, crude) was obtained as a colorless oil. LC-MS: (ES) m/z 547.3 (M+H⁺).

Step h) To a solution of 1-methylindazol-5-amine (43.75 mg, 297.30 mol) in DCE (1 mL) was added Al(CH₃)₃ (in toluene) (2 M, 178.38 μL) at 0° C. After stirring for 30 min, a solution of methyl 1-(2-fluoro-6-methyl-benzoyl)-2-[4-[[(2R)-2-(trifluoromethyl)pyrrolidin-1-yl] methyl]phenyl]-2,3,4,4a,5,6,7,7a-octahydrocyclopenta[b]pyridine-3-carboxylate (65 mg, 118.92 mol) in DCE (1 mL) was added. The mixture was stirred at 85° C. for 4 h. The reaction was concentrated to get a residue. The residue was purified by prep-HPLC (column: Phenomenex Gemini-NX 80*40 mm*3 m; mobile phase: [water (0.05% NH₃H₂O+10 mM NH₄HCO₃)—CA N]; B %: 60%-90%, 8 min). 1-(2-fluoro-6-methyl-benzoyl)-N-(1-methylindazol-5-yl)-2-[4-[[(2R)-2-(trifluoromethyl)pyrrolidin-1-yl]methyl]phenyl]-2,3,4,4a,5,6,7,7a-octahydrocyclopenta[b]pyridine-3-carboxamide (10 mg, 14.81 mol, 12.45% yield, 98% purity) was obtained as a white solid. ¹H NMR (400 MHz, METHANOL-d₄) δ ppm 1.15-1.38 (m, 2H), 1.39-1.62 (m, 2H), 1.64-1.84 (m, 3H), 1.87-2.32 (m, 6H), 2.34-2.49 (m, 3H), 2.80-2.92 (m, 1H), 3.16 (br dd, J=9.79, 5.52 Hz, 1H), 3.34 (br s, 2H), 3.48-3.67 (m, 1H), 3.72-3.97 (m, 1H), 3.98-4.17 (m, 4H), 6.65-6.84 (m, 1H), 7.01-7.21 (m, 2H), 7.29 (br d, J=8.28 Hz, 2H), 7.34-7.58 (m, 3H), 7.63-7.87 (m, 2H), 7.93 (d, J=11.80 Hz, 1 H). LC-MS: (ES) m/z 662.3 (M+H⁺).

### Example S134: Synthesis of cis-1-(2-fluoro-6-methylbenzoyl)-N-(1-methyl-1H-indazol-5-yl)-2-(4-((1-methylpiperidin-4-yl)amino)phenyl)octahydro-1H-cyclopenta[b]pyridine-3-carboxamide (Compound No. 117)

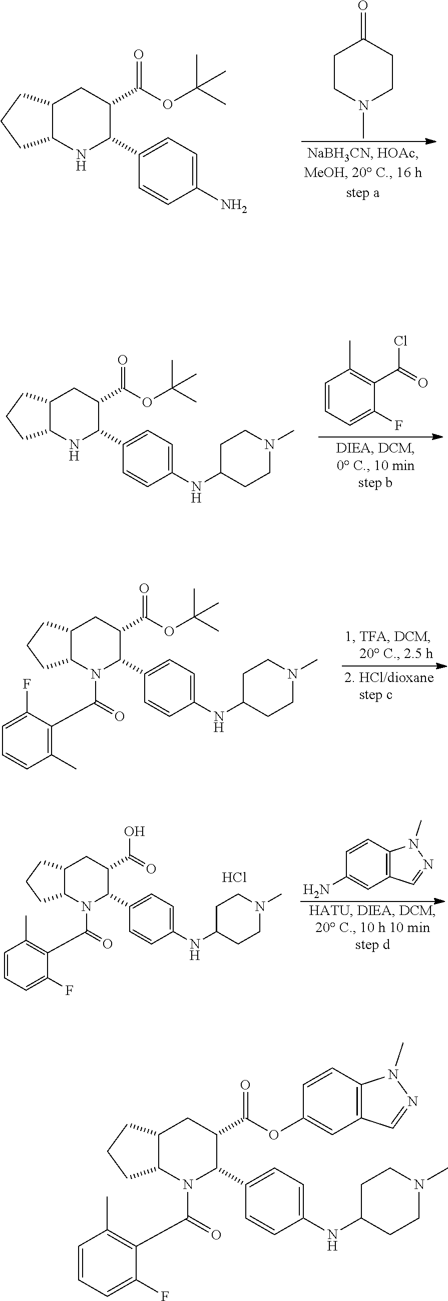

Step a) To a mixture of tert-butyl 2-(4-aminophenyl)-2,3,4,4a,5,6,7,7a-octahydro-1H-cyclopenta[b]pyridine-3-carboxylate (150 mg, 474.03 mol) and 1-methylpiperidin-4-one (64.37 mg, 568.83 mol, 66.15 μL) in MeOH (5 mL) was added AcOH (28.47 mg, 474.03 mol, 27.11 μL) and NaBH₃CN (89.37 mg, 1.42 mmol) at 20° C. under N₂, the mixture was stirred at 20° C. for 16 h. The reaction mixture was concentrated under reduced pressure to remove solvent. The residue was diluted with saturated NaHCO₃(aq) (20 mL) and extracted with EtOAc (20 mL*2). The combined organic layers were dried, filtered and concentrated under reduced pressure to give a residue. The residue was purified by prep-TLC (SiO₂, DCM:methanol:NH₃·H₂O=10:1:0.1) to get tert-butyl 2-[4-[(1-methyl-4-piperidyl)amino]phenyl]-2,3,4,4a,5,6,7,7a-octahydro-1H-cyclopenta[b]pyridine-3-carboxylate (75 mg, 172.27 umol, 36.34% yield, 95% purity) as a light yellow oil. ¹H NMR (400 MHz, CDCl₃) δ ppm 1.18 (s, 9H), 1.39-1.64 (m, 6H), 1.72-1.95 (m, 10H), 1.99-2.09 (m, 5H), 2.11-2.20 (m, 3H), 2.31 (s, 3H), 2.76-2.88 (m, 3H), 3.22-3.38 (m, 2H), 3.91 (d, J=5.50 Hz, 1H), 6.54 (d, J=8.63 Hz, 2H), 7.14 (d, J=8.38 Hz, 2H). LC-MS: (ES) m z 414.3 (M+H⁺).

Step b) To a mixture of tert-butyl 2-[4-[(1-methyl-4-piperidyl)amino]phenyl]-2,3,4,4a,5,6,7,7a-octahydro-1H-cyclopenta[b]pyridine-3-carboxylate (70 mg, 169.25 mol) and DIEA (43.75 mg, 338.50 mol, 58.96 μL) in DCM (3 mL) was added 2-fluoro-6-methyl-benzoyl chloride (27.75 mg, 160.79 mol) at 0° C. under N₂. The mixture was stirred at 0° C. for 10 min. The reaction mixture was concentrated to get a residue. The residue was purified by prep-TLC (SiO₂, DCM:methanol:NH₃·H₂O=10:1:0.1) to give tert-butyl 1-(2-fluoro-6-methyl-benzoyl)-2-[4-[(1-methyl-4-piperidyl)amino]phenyl]-2,3,4,4a,5,6,7,7a-octahydrocyclopenta[b]pyridine-3-carboxylate (64 mg, 116.42 mol, 68.79% yield, 100% purity) as a colorless oil. LC-MS: (ES) m/z 550.3 (M+H⁺).

Step c) To a mixture of tert-butyl 1-(2-fluoro-6-methyl-benzoyl)-2-[4-[(1-methyl-4-piperidyl)amino]phenyl]-2,3,4,4a,5,6,7,7a-octahydrocyclopenta[b]pyridine-3-carboxylate (64 mg, 116.42 μmol) in DCM (5 mL) was added TFA (3.08 g, 27.01 mmol, 2 mL) at 20° C. under N₂. The mixture was stirred at 20° C. for 2.5 h. The mixture was concentrated to get a residue, then 10 mL (4 M HCl/dioxane) was added, and stirred at 20° C. for 10 min, then concentrated to get 1-(2-fluoro-6-methyl-benzoyl)-2-[4-[(1-methyl-4-piperidyl)amino]phenyl]-2,3,4,4a,5,6,7,7a-octahydrocyclopenta-[b]pyridine-3-carboxylic acid (60 mg, 113.19 μmol, 97.22% yield, HCl) as a light yellow oil. LC-MS: (ES) m/z 494.3 (M+H⁺).

Step d) To a mixture of 1-(2-fluoro-6-methyl-benzoyl)-2-[4-[(1-methyl-4-piperidyl)amino] phenyl]-2,3,4,4a,5,6,7,7a-octahydrocyclopenta[b]pyridine-3-carboxylic acid (60 mg, 113.19 μmol, HCl) in DCM (3 mL) was added HATU (51.65 mg, 135.83 mol) and DIEA (43.89 mg, 339.58 μmol, 59.15 μL) at 20° C. under N₂. The mixture was stirred at 20° C. for 10 min, then 1-methylindazol-5-amine (24.99 mg, 169.79 mol) was added and the mixture was stirred at 20° C. for 10 hr. The mixture was concentrated to get a residue. The residue was purified by Prep-HPLC (column: Phenomenex Gemini-NX 150*30 mm*5 μm; mobile phase: [water (0.05% HCl)-ACN]; B %: 18%-58%, 10 min to give 1-(2-fluoro-6-methyl-benzoyl)-N-(1-methylindazol-5-yl)-2-[4-[(1-methyl-4-piperidyl)amino]phenyl]-2,3,4,4a,5,6,7,7a-octahydrocyclopenta[b]pyridine-3-carboxamide (20 mg, 30.34 mol, 26.80% yield, 100% purity, HCl) as a brown solid. ¹H NMR (400 MHz, METHANOL-d₄) δ ppm 1.18-1.45 (m, 3H), 1.48-1.64 (m, 2H), 1.78 (br s, 1H), 2.01-2.16 (m, 4H), 2.22-2.35 (m, 3H), 2.44 (s, 2H), 2.88 (s, 3H), 3.04-3.16 (m, 2H), 3.18-3.28 (m, 1H), 3.51-3.70 (m, 3H), 3.77-3.96 (m, 2H), 4.05-4.11 (m, 3H), 6.58-6.74 (m, 1H), 7.03-7.22 (m, 2H), 7.36-7.58 (m, 5H), 7.75-8.11 (m, 4H). LC-MS: (ES) m/z 623.3 (M+H⁺).

### Example S135: Synthesis of cis-2-(4-(cyclopentylamino)phenyl)-1-(2-fluoro-6-methylbenzoyl)-N-(4-methyl-3-(trifluoromethyl)phenyl)octahydrofuro[3,4-b]pyridine-3-carboxamide (Compound No. 118)

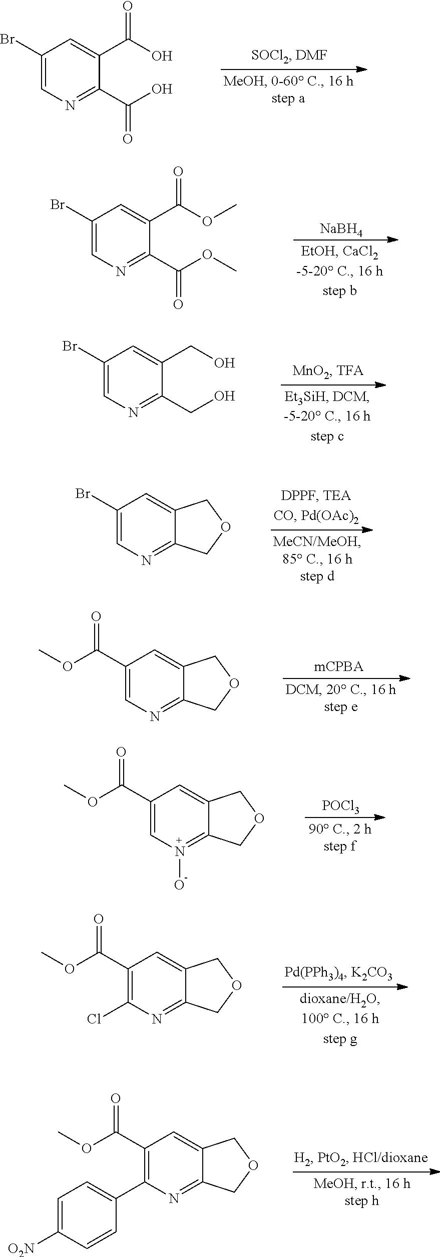

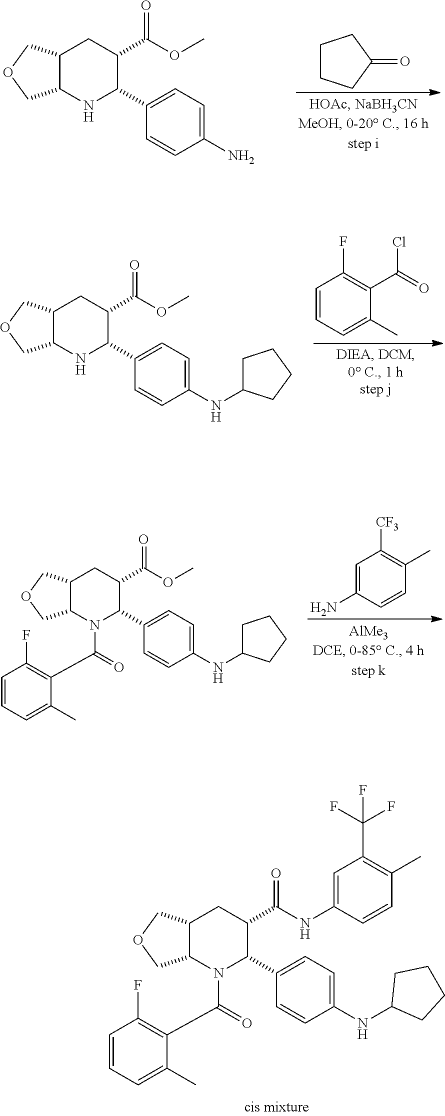

Step a) To a solution of 5-bromopyridine-2,3-dicarboxylic acid (50 g, 203.24 mmol) in MeOH (500 mL) was added SOCl₂ (145.08 g, 1.22 mol, 88.46 mL) and DMF (2.97 g, 40.65 mmol, 3.13 mL) at 0° C. The mixture was stirred at 60° C. for 16 h. The reaction mixture was concentrated under reduced pressure to give a residue. The residue was purified by column chromatography (SiO₂, petroleum ether/ethyl acetate=10/1 to 4/1) to give compound dimethyl 5-bromopyridine-2,3-dicarboxylate (47.5 g, 173.31 mmol, 85.28% yield, 100% purity) as a light yellow solid. ¹H NMR (400 MHz, DMSO-d₆) δ ppm 4.53 (br s, 2H), 4.64 (d, J=4.89 Hz, 2H), 5.17 (br s, 1H), 5.43 (br t, J=5.50 Hz, 1H), 7.94-8.02 (m, 1H), 8.50 (d, J=2.20 Hz, 1H). LC-MS: (ES) m/z 273.9 (M+H⁺).

Step b) To a solution of dimethyl 5-bromopyridine-2,3-dicarboxylate (42 g, 153.25 mmol) in EtOH (500 mL) was slowly added NaBH₄ (28.99 g, 766.23 mmol) at −5° C. Then the CaCl₂) (15.31 g, 137.92 mmol) in EtOH (150 mL) was added dropwise slowly at −5° C. The mixture was stirred for at 20° C. for 16 h. The mixture was quenched by slow addition of aqueous 2 N HCl solution (500 mL, pH-2-3). After stirring for 2 h, the mixture was concentrated to give the residue. Saturated aqueous sodium bicarbonate solution was added to the residue until pH=7. The aqueous mixture was extracted with EtOAc 900 mL (450 mL×2). The combined organic layers were dried over Na₂SO₄, filtered and concentrated under reduced pressure to give a crude product. The crude product was purified by column chromatography (SiO₂, DCM:methanol:NH₃·H₂O=50:1:0.1 to 10:1:0.1, plate 2) to give [5-bromo-2-(hydroxymethyl)-3-pyridyl]methanol (22 g, 100.90 mmol, 65.84% yield) as a light yellow solid. ¹H NMR (400 MHz, DMSO-d₆) δ 4.53 (s, 2H), 4.64 (s, 2H), 7.99 (d, J=1.71 Hz, 1H), 8.50 (d, J=2.20 Hz, 1H). LC-MS: (ES) m/z 217.9 (M+H⁺).

Step c) To a solution of [5-bromo-2-(hydroxymethyl)-3-pyridyl]methanol (21 g, 96.31 mmol) in DCM (500 mL) was added MnO₂ (41.87 g, 481.55 mmol) at −5° C., then TFA (164.72 g, 1.44 mol, 106.96 mL) was added. Then triethylsilane (50.39 g, 433.39 mmol, 69.22 mL) was added dropwise over 15 min. Then the mixture was stirred at 0° C. for 1 h. The mixture was stirred at 20° C. for 14 h and 45 min. 50 ml of H₂O was added to the reaction mixture, then filtered and concentrated under reduced pressure to give a residue. The residue was alkalified with aqeuous NaHCO₃ (200 mL) to pH-7-8 and extracted with DCM (500 mL×2). The combined organic layers were dried over anhydrous Na₂SO₄, filtered and concentrated under reduced pressure to give a residue. The residue was purified by column chromatography (SiO₂, petroleum ether/ethyl acetate=3/1 to 0/1, plate 2) to give 3-bromo-5,7-dihydrofuro[3,4-b]pyridine (4.7 g, 22.56 mmol, 23.42% yield, 96% purity) as a white solid. ¹H NMR (400 MHz, CDCl₃) δ 5.02 (d, J=1.71 Hz, 2H), 5.15 (d, J=0.73 Hz, 2H), 7.69 (s, 1H), 8.54 (d, J=0.98 Hz, 1H). LC-MS: (ES) m/z 200.0 (M+H⁺).

Step d) A mixture of 3-bromo-5,7-dihydrofuro[3,4-b]pyridine (5.7 g, 28.50 mmol), DPPF (4.74 g, 8.55 mmol), TEA (8.65 g, 85.49 mmol, 11.90 mL) and Pd(OAc)₂ (959.62 mg, 4.27 mmol) in MeOH (20 mL) and MeCN (50 mL) was degassed and purged with CO (50 psi) 3 times, and then the mixture was stirred at 80° C. for 32 h under CO atmosphere. The reaction mixture was filtered and concentrated under reduced pressure to give a residue. The residue was purified by column chromatography (SiO₂, petroleum ether/ethyl acetate=3/1 to 3/2) to give methyl 5,7-dihydrofuro[3,4-b] pyridine-3-carboxylate (4.3 g, 23.04 mmol, 80.85% yield, 96% purity) as a light yellow solid. ¹H NMR (400 MHz, CDCl₃) δ 3.97 (s, 3H), 5.12 (t, J=1.83 Hz, 2H), 5.21 (s, 2H), 8.17 (s, 1H), 9.12 (s, 1H). LC-MS: (ES) m/z 180.1 (M+H⁺).

Step e) To a solution of methyl 5,7-dihydrofuro[3,4-b]pyridine-3-carboxylate (4.3 g, 24.00 mmol) in DCM (70 mL) was added m-CPBA (9.01 g, 44.40 mmol, 85% purity) at 0° C. The mixture was stirred at 20° C. for 16 h. The reaction mixture was quenched by addition of Na₂SO₃ (10%) 45 mL at 0° C., and then extracted with DCM (100 mL×2). The combined organic layers were washed with aqueous NaHCO₃ 40 mL, dried over anhydrous Na₂SO₄, filtered and concentrated under reduced pressure to give the residue methyl 1-oxido-5,7-dihydrofuro[3,4-b]pyridin-1-ium-3-carboxylate (4.8 g, crude) as light yellow solid, which was used in next step without further purification. ¹H NMR (400 MHz, CDCl₃) δ 3.98 (s, 3 H), 5.24-5.30 (m, 4H), 7.74 (s, 1H), 8.70 (s, 1H). LC-MS: (ES) m/z 196.1 (M+H⁺).

Step f) The methyl 1-oxido-5,7-dihydrofuro[3,4-b]pyridin-1-ium-3-carboxylate (2 g, 8.20 mmol) was added to POCl₃ (27.90 g, 181.93 mmol, 16.91 mL). The mixture was stirred at 90° C. for 2 h. The reaction mixture was concentrated under reduced pressure to remove POCl₃. The residue was alkalified with aqueous NaHCO₃ (80 mL) and extracted with EtOAc (150 mL×2). The combined organic layers were dried over anhydrous Na₂SO₄, filtered and concentrated under reduced pressure to give a residue. The residue was purified by flash silica gel chromatography (ISCO®; 20 g SepaFlash® Silica Flash Column, eluent of 0˜20% ethyl acetate/petroleum ether gradient@35 mL/min) to give methyl 2-chloro-5,7-dihydrofuro[3,4-b] pyridine-3-carboxylate (750 mg, 3.44 mmol, 41.97% yield, 98% purity) as light yellow solid. ¹H NMR (400 MHz, CDCl₃) δ 3.97 (s, 3H), 5.07 (t, J=1.83 Hz, 2H), 5.18 (s, 2H), 8.05 (s, 1H). LC-MS: (ES) m/z 214.1 (M+H⁺).

Step g) A mixture of methyl 2-chloro-5,7-dihydrofuro[3,4-b]pyridine-3-carboxylate (2.1 g, 9.83 mmol), (4-nitrophenyl)boronic acid (2.95 g, 17.70 mmol), Pd(PPh₃)₄ (1.14 g, 983.07 mol) and K₂CO₃ (2 M, 17.20 mL) in dioxane (50 mL) was degassed and purged with N₂ 3 times, and then the mixture was stirred at 100° C. for 16 h under N₂ atmosphere. The reaction mixture was concentrated under reduced pressure to remove dioxane and then extracted with EtOAc 160 mL (80 mL×2). The combined organic layers were washed with brine (20 mL), dried over anhydrous Na₂SO₄, filtered and concentrated under reduced pressure to give a residue. The residue was purified by column chromatography (SiO₂, petroleum ether/ethyl acetate=5/1 to 2/1, plate 2) to give methyl 2-(4-nitrophenyl)-5,7-dihydrofuro[3,4-b]pyridine-3-carboxylate (2.6 g, 6.93 mmol, 70.47% yield, 80% purity) as a light brown solid. ¹H NMR (400 MHz, CDCl₃) δ 3.74 (s, 3H), 5.16 (d, J=1.47 Hz, 2H), 5.28 (s, 2H), 7.67-7.69 (m, 2H), 8.11 (s, 1H), 8.32 (d, J=8.80 Hz, 2H). LC-MS: (ES) m/z 301.1 (M+H⁺).

Step h) A mixture of methyl 2-(4-nitrophenyl)-5,7-dihydrofuro[3,4-b]pyridine-3-carboxylate (1.68 g, 4.76 mmol), PtO₂ (431.98 mg, 1.90 mmol) and HCl/dioxane (4 M, 2.38 mL) in MeOH (50 mL) was degassed and purged with H₂ (15 psi) 3 times, and then the mixture was stirred at 20° C. for 4 h under H₂ atmosphere. The reaction mixture was filtered and concentrated under reduced pressure to give a residue. The residue was alkalified with aqueous NaHCO₃ 10 mL and extracted with DCM (50 mL×2). The combined organic layers were dried over anhydrous Na₂S04, filtered and concentrated under reduced pressure to give a crude product. The crude product combined with previous batch was purified by column chromatography (SiO₂, DCM:methanol:NH₃·H₂O=100:1:0.1-20:1:0.1) to give cis-methyl 2-(4-amino phenyl)-1,2,3,4,4a,5,7,7a-octahydrofuro[3,4-b] pyridine-3-carboxyl ate (90% purity) (1.67 g) as a light yellow gum. ¹H NMR (400 MHz, CDCl₃) δ 2.12-2.20 (m, 2H), 2.21-2.28 (m, 1H), 2.87-2.93 (m, 1H), 3.38 (s, 3H), 3.50-3.55 (m, 2H), 3.70-3.81 (m, 2H), 3.85-3.93 (m, 1H), 3.96-4.04 (m, 2H), 6.61-6.67 (m, 2H), 7.05-7.13 (m, 2H). LC-MS: (ES) m/z 277.2 (M+H⁺).

Step i) To a solution of cis-methyl 2-(4-aminophenyl)-1,2,3,4,4a,5,7,7a-octahydrofuro-[3,4-b] pyridine-3-carboxylate (800 mg, 2.90 mmol) and cyclopentanone (304.00 mg, 3.61 mmol, 320.00 μL) in MeOH (20 mL) was added CH₃COOH (208.63 mg, 3.47 mmol, 198.69 μL, 1.2 eq) and NaBH₃CN (727.73 mg, 11.58 mmol) at 0° C. The mixture was stirred at 20° C. for 16 h. Another portion of NaBH₃CN (181.93 mg, 2.90 mmol) and CH₃COOH (105.00 mg, 1.75 mmol, 100 μL) were added to the mixture, then stirred at 20° C. for another 4 h. The reaction mixture was concentrated under reduced pressure to give a residue. The residue was alkalified with aqueous NaHCO₃ (10 mL) and extracted with DCM (50 mL×2). The combined organic layers were dried over anhydrous Na₂SO₄, filtered and concentrated under reduced pressure to give a residue. The residue was purified by column chromatography (SiO₂, DCM:methanol=100/1, plate 2) to give cis-methyl 2-[4-(cyclopentylamino)phenyl]-1,2,3,4,4a,5,7,7a-octahydrofuro [3,4-b]pyridine-3-carboxylate (820 mg, 2.14 mmol, 74.01% yield, 90% purity) as a light yellow gum. ¹H NMR (400 MHz, CDCl₃) δ 1.44 (br dd, J=11.74, 6.11 Hz, 2H), 1.59-1.64 (m, 2H), 1.69-1.73 (m, 2H), 1.95-2.06 (m, 3H), 2.11-2.19 (m, 2H), 2.20-2.28 (m, 1H), 2.89 (q, J=5.95 Hz, 1H), 3.37 (s, 3 H), 3.51-3.55 (m, 1H), 3.73-3.80 (m, 3H), 3.86-3.92 (m, 1H), 3.96-4.04 (m, 2H), 6.51-6.57 (m, 2H), 7.11 (d, J=8.56 Hz, 2H). LC-MS: (ES) m/z 345.2 (M+H⁺).

Step j) To a solution of cis-methyl 2-[4-(cyclopentylamino)phenyl]-1,2,3,4,4a,5,7,7a-octahydrofuro[3,4-b]pyridine-3-carboxylate (200 mg, 580.64 mol) in DCM (10 mL) was added DIEA (262.65 mg, 2.03 mmol, 353.98 μL), then the 2-fluoro-6-methyl-benzoyl chloride (100.21 mg, 580.64 mol) in DCM (1 mL) was added dropwise. The mixture was stirred at 0° C. for 3 h. The reaction mixture was quenched by addition of H₂O (10 mL), and then extracted with DCM (30 mL×2). The combined organic layers were dried over anhydrous Na₂SO₄, filtered and concentrated under reduced pressure to give a residue. The residue was purified by column chromatography (SiO₂, DCM:methanol=50/1, plate 2) to give the crude product. The crude product was further purified by prep-HPLC (HCl condition; column: Phenomenex Gemini-NX 150×30 mm×5 μm; mobile phase: [water (0.05% HCl)-ACN]; B %: 25%-55%, 7 min). The eluent was alkalified with aqueous NaHCO₃ (10 mL) and extracted with EtOAc (50 mL×2). The combined organic layers were dried over anhydrous Na₂SO₄, filtered and concentrated under reduced pressure to give the pure product cis-methyl 2-[4-(cyclo pentylamino)phenyl]-1-(2-fluoro-6-methyl-benzoyl)-3,4,4a,5,7,7a-hexahydro-2H-furo[3,4-b]pyridine-3-carboxylate (110 mg, 224.32 umol, 38.63% yield, 98% purity) as light yellow solid. ¹H NMR (400 MHz, CDCl₃) δ 0.82-0.93 (m, 2H), 1.45 (ddd, J=19.45, 13.05, 6.15 Hz, 3H), 1.62 (br d, J=5.27 Hz, 2H), 1.68-1.76 (m, 2H), 2.02 (br dd, J=12.92, 6.15 Hz, 2H), 2.29-2.38 (m, 3H), 2.85-3.06 (m, 1H), 3.08-3.18 (m, 1H), 3.31 (brt, J=8.53 Hz, 1H), 3.43 (s, 1H), 3.68 (s, 1H), 3.71-3.74 (m, 2H), 3.74-3.86 (m, 2H), 3.91-4.10 (m, 1H), 4.16-4.29 (m, 1H), 4.95-5.03 (m, 1H), 6.30-6.40 (m, 1H), 6.49-6.60 (m, 2H), 6.63-6.81 (m, 1H), 6.92-7.07 (m, 1H), 6.92-7.07 (m, 1H), 7.15-7.26 (m, 2H). LC-MS: (ES) m/z 481.3 (M+H⁺).

Step k) To a solution of 4-methyl-3-(trifluoromethyl)aniline (111.57 mg, 624.26 mol, 91.45 μL) in DCE (1 mL) was added Al(CH₃)₃ (in toulene) (2 M, 364.15 μL) at 0° C., after 20 min, the cis-methyl 2-[4-(cyclopentylamino)phenyl]-1-(2-fluoro-6-methyl-benzoyl)-3,4,4a,5,7,7a-hexahydro-2H-furo[3,4-b]pyridine-3-carboxylate (100 mg, 208.09 mol) in DCE (1 mL) was added. The mixture was stirred at 85° C. for 3 hr 40 min. The reaction mixture was diluted with aq. NaHCO₃ 8 mL and extracted with DCM 80 mL (40 mL×2). The combined organic layers were dried over anhydrous Na₂SO₄, filtered and concentrated under reduced pressure to give a residue. The residue was purified by column chromatography (SiO₂, petroleum ether/ethyl acetate=200/1) to give the crude product. The crude product was further purified by prep-HPLC (HCl condition, column: Phenomenex Gemini-NX 150×30 mm×5 μm; mobile phase: [water (0.05% HCl)-ACN]; B %: 35%-65%, 7 min) to give cis-2-[4-(cyclopentylamino)phenyl]-1-(2-fluoro-6-methyl-benzoyl)-N-[4-methyl-3-(trifluoromethyl)phenyl]-3,4,4a,5,7,7a-hexahydro-2H-furo[3,4-b]pyridine-3-carboxamide (40 mg, 59.38 mol, 28.54% yield, 98% purity, HCl) as a white solid. ¹H NMR (400 MHz, METHANOL-d₄) δ 1.27-1.42 (m, 2H), 1.57-1.73 (m, 4H), 1.81 (br s, 2H), 1.91-2.07 (m, 3H), 2.08-2.21 (m, 1H), 2.25-2.47 (m, 5H), 2.66 (s, 1H), 3.11-3.26 (m, 1H), 3.44-3.74 (m, 1H), 3.75-3.95 (m, 2H), 3.98-4.13 (m, 1H), 4.20-4.33 (m, 1H), 6.29-6.52 (m, 1H), 6.73-6.88 (m, 1H), 6.94 (br d, J=8.07 Hz, 1H), 7.00-7.44 (m, 5H), 7.46-7.60 (m, 1H), 7.65-7.87 (m, 1H), 9.66-9.82 (m, 0.4 H), 10.25 (s, 0.3 H). LC-MS: (ES) m/z 624.3 (M+H⁺).

### Example S136: Synthesis of (2R,3S,4aR,7aS)-2-(4-(cyclopentylamino)phenyl)-1-(2-fluoro-6-methylbenzoyl)-N-(4-methyl-3-(trifluoromethyl)phenyl)octahydrofuro[3,4-b]pyridine-3-carboxamide and (2S,3R,4aS,7aR)-2-(4-(cyclopentylamino)phenyl)-1-(2-fluoro-6-methylbenzoyl)-N-(4-methyl-3-(trifluoromethyl)phenyl)octahydrofuro[3,4-b]pyridine-3-carboxamide (Compound Nos. 119 and 120)

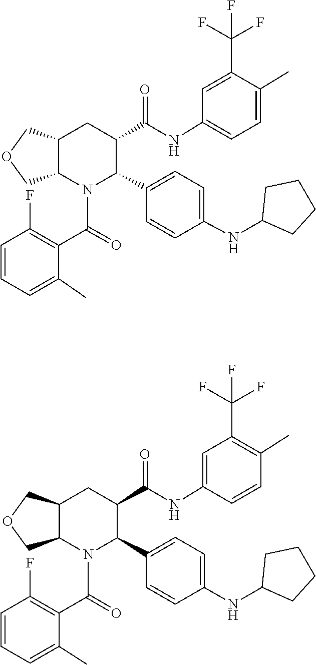 [SMILES: Cc1ccc(NC(=O)[C@H]2C[C@H]3COC[C@H]3N(C(=O)c3c(C)cccc3F)[C@H]2c2ccc(NC3CCCC3)cc2)cc1C(F)(F)F ; Cc1ccc(NC(=O)[C@@H]2C[C@@H]3COC[C@@H]3N(C(=O)c3c(C)cccc3F)[C@@H]2c2ccc(NC3CCCC3)cc2)cc1C(F)(F)F]

The cis-methyl2-[4-(cyclopentylamino)phenyl]-1,2,3,4,4a,5,7,7a-octahydrofuro[3,4-b] pyridine-3-carboxylate (800 mg, 2.32 mmol) was separated by SFC (column: DAICEL CHIRALPAK IG (250 mm×30 mm×10 m); mobile phase: [0.1% NH₃·H₂O ETOH]; B %: 40%-40%, 8 min). The compound methyl(2R,3S,4aR,7aS)-2-[4-(cyclopentylamino)phenyl]-1,2,3,4, 4a,5,7,7a-octahydrofuro[3,4-b]pyridine-3-carboxylate (peak 1 on SFC spectrum, 245 mg, 704.17 umol, 30.32% yield, 99% purity) was obtained as a light yellow solid. ¹H NMR (400 MHz, CDCl₃) δ 1.39-1.50 (m, 2H), 1.54-1.66 (m, 2H), 1.66-1.77 (m, 2H), 1.95-2.06 (m, 2H), 2.12-2.19 (m, 2H), 2.20-2.29 (m, 1H), 2.89 (q, J=5.79 Hz, 1H), 3.51-3.57 (m, 1H), 3.72-3.82 (m, 3H), 3.86-3.93 (m, 1H), 3.96-4.04 (m, 2H), 6.54 (d, J=8.56 Hz, 2H), 7.11 (d, J=8.56 Hz, 2H). LC-MS: (ES) m/z 345.2 (M+H⁺). The compound methyl(2S,3R,4aS,7aR)-2-[4-(cyclopentylamino) phenyl]-1,2,3,4,4a,5,7,7a-octahydrofuro[3,4-b]pyridine-3-carboxylate (peak 2, 310 mg, 890.99 umol, 38.36% yield, 99% purity) was obtained as light yellow solid. ¹H NMR (400 MHz, CDCl₃) δ 1.45 (dt, J=12.41, 6.14 Hz, 2H), 1.54-1.65 (m, 2H), 1.66-1.77 (m, 2H), 2.00 (dt, J=12.47, 6.24 Hz, 2H), 2.16 (q, J=5.87 Hz, 2H), 2.22-2.31 (m, 1H), 2.89 (q, J=5.87 Hz, 1 H), 3.38 (s, 3H), 3.51-3.57 (m, 1H), 3.71-3.81 (m, 3H), 3.86-3.93 (m, 1H), 3.97-4.04 (m, 2H), 6.54 (d, J=8.56 Hz, 2H), 7.11 (d, J=8.56 Hz, 2H). LC-MS: (ES) m/z 345.2 (M+H⁺).

The title compounds were synthesized in similar fashion as Example S135.

### (2R,3S,4aR,7aS)-2-(4-(cyclopentylamino)phenyl)-1-(2-fluoro-6-methylbenzoyl)-N-(4-methyl-3-(trifluoromethyl)phenyl)octahydrofuro [3,4-b]pyridine-3-carboxamide

¹H NMR (400 MHz, METHANOL-d₄) δ 1.39 (s, 1H), 1.68 (br s, 4H), 1.83 (br s, 2H), 1.93-2.05 (m, 3H), 2.08-2.27 (m, 1H), 2.34-2.47 (m, 5H), 2.77 (br d, J=7.09 Hz, 1H), 3.12-3.26 (m, 1H), 3.34-3.58 (m, 1H), 3.63-3.79 (m, 1H), 3.81-3.97 (m, 2H), 3.98-4.12 (m, 1H), 4.20-4.39 (m, 1H), 5.04-5.33 (m, 1H), 6.39-6.54 (m, 0.5 H), 6.68-6.77 (m, 0.5 H), 6.88-7.18 (m, 4H), 7.20-7.25 (m, 1H), 7.26-7.50 (m, 3H), 7.51-7.65 (m, 1H), 7.70-7.83 (m, 1H), 9.76-9.90 (m, 0.3 H), 10.29 (s, 0.2 H). m/z 624.4 (M+H⁺).

### (2S,3R,4aS,7aR)-2-(4-(cyclopentylamino)phenyl)-1-(2-fluoro-6-methylbenzoyl)-N-(4-methyl-3-(trifluoromethyl)phenyl)octahydrofuro[3,4-b]pyridine-3-carboxamide

¹H NMR (400 MHz, METHANOL-d₄) δ ppm 1.39 (s, 1H), 1.68 (br s, 4H), 1.83 (br s, 2H), 1.87-2.07 (m, 4H), 2.07-2.30 (m, 1H), 2.34-2.56 (m, 5H), 2.58-2.93 (m, 1H), 3.12-3.27 (m, 1H), 3.68-3.81 (m, 1H), 3.82-3.97 (m, 2H), 3.97-4.13 (m, 1H), 4.18-4.36 (m, 1H), 5.07-5.28 (m, 1H), 6.28-6.57 (m, 1H), 6.67-6.95 (m, 1H), 6.97-7.44 (m, 7H), 7.45-7.66 (m, 1H), 7.69-7.88 (m, 1H), 9.74-9.96 (m, 0.4 H), 10.29 (s, 0.2 H); LC-MS: (ES) m/z 624.4 (M+H⁺).

### Example S137: Synthesis of (2R,3S,4aR,7aS)-2-(4-(cyclopentylamino)phenyl)-1-(2-fluoro-6-methylbenzoyl)-N-(1-methyl-1H-indazol-5-yl)octahydrofuro[3,4-b]pyridine-3-carboxamide (Compound No. 126)

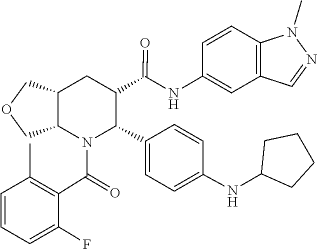

The title compound was synthesized in similar fashion as Examples S135 and S136. ¹H NMR (400 MHz, METHANOL-d₄) δ 1.59-1.76 (m, 4H), 1.82 (br s, 2H), 1.94 (br d, J=16.06 Hz, 2H), 2.03-2.10 (m, 1H), 2.11-2.31 (m, 1H), 2.45 (d, J=13.05 Hz, 2H), 2.53-2.85 (m, 1H), 3.19-3.28 (m, 1H), 3.34-3.59 (m, 1H), 3.69-3.82 (m, 1H), 3.85-3.97 (m, 2H), 3.98-4.05 (m, 4H), 4.06-4.14 (m, 1H), 4.21-4.35 (m, 1H), 6.34-6.78 (m, 1H), 7.02-7.23 (m, 3H), 7.23-7.35 (m, 2H), 7.36-7.54 (m, 3H), 7.62 (d, J=7.78 Hz, 1 H), 7.78-7.96 (m, 2H). LC-MS: (ES) m/z 596.3 (M+H⁺).

### Example S138: Synthesis of (2R,3S,4aR,7aS)-2-(4-(cyclopentylamino)phenyl)-1-(2-fluoro-6-methylbenzoyl)-N-(1-methyl-1H-indazol-6-yl)octahydrofuro[3,4-b]pyridine-3-carboxamide (Compound No. 122)

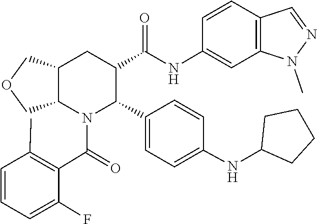

The title compound was synthesized in similar fashion as Examples S135 and S136. ¹H NMR (400 MHz, METHANOL-d₄) δ 1.66 (br s, 4H), 1.82 (br s, 2H), 1.97 (br s, 2 H), 2.03-2.11 (m, 1H), 2.15-2.31 (m, 1H), 2.46 (d, J=14.92 Hz, 2H), 2.56-2.84 (m, 1 H), 3.20-3.29 (m, 1H), 3.35-3.59 (m, 1H), 3.69-3.84 (m, 1H), 3.84-3.93 (m, 3H), 3.93-4.05 (m, 3H), 4.05-4.15 (m, 1H), 4.21-4.35 (m, 1H), 6.33-6.76 (m, 1H), 6.90-7.07 (m, 2H), 7.07-7.34 (m, 4H), 7.35-7.62 (m, 2H), 7.64-7.70 (m, 1H), 7.77-7.95 (m, 2 H). LC-MS: (ES) m/z 596.3 (M+H⁺).

### Example S139: Synthesis of (2R,3S,4aR,7aS)-2-(4-(cyclopentylamino)phenyl)-1-(2-fluoro-6-methylbenzoyl)-N-(1-methyl-1H-pyrazolo[4,3-b]pyridin-6-yl)octahydrofuro[3,4-b]pyridine-3-carboxamide (Compound No. 123)

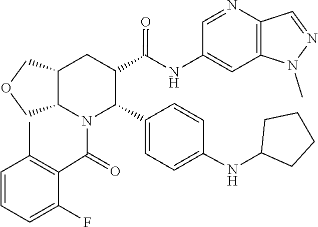

The title compound was synthesized in similar fashion as Examples S135 and S136. ¹H NMR (400 MHz, METHANOL-d₄) δ 1.69 (br s, 4H), 1.84 (br s, 2H), 1.97 (br s, 2 H), 2.06-2.15 (m, 1H), 2.19-2.36 (m, 1H), 2.43-2.52 (m, 2H), 2.53-2.89 (m, 1H), 3.33-3.41 (m, 2H), 3.69-4.06 (m, 4H), 4.06-4.11 (m, 2H), 4.12-4.19 (m, 2H), 4.23-4.35 (m, 1H), 4.99-5.23 (m, 1H), 6.43-6.79 (m, 1H), 7.05-7.17 (m, 2H), 7.18-7.34 (m, 2 H), 7.38-7.50 (m, 2H), 7.84 (br d, J=8.56 Hz, 1H), 8.20-8.30 (m, 1H), 8.39-8.89 (m, 2 H). LC-MS: (ES) m/z 597.3 (M+H⁺).

### Example S140: Synthesis of (2R,3S,4aR,7aS)-2-(4-(cyclopentylamino)phenyl)-N-(4-(dimethylamino)phenyl)-1-(2-fluoro-6-methylbenzoyl)octahydrofuro[3,4-b]pyridine-3-carboxamide (Compound No. 124)

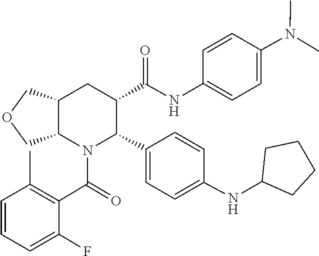 [SMILES: Cc1cccc(F)c1C(=O)N1[C@@H]2COC[C@@H]2C[C@H](C(=O)Nc2ccc(N(C)C)cc2)[C@@H]1c1ccc(NC2CCCC2)cc1]

The title compound was synthesized in similar fashion as Examples S135 and S136. ¹H NMR (400 MHz, METHANOL-d₄) δ ppm 1.39 (s, 1H), 1.71 (br s, 4H), 1.86 (br s, 2H), 1.92-2.06 (m, 3H), 2.07-2.49 (m, 3H), 2.50-2.83 (m, 1H), 3.23 (s, 3H), 3.27 (s, 3 H), 3.34-3.53 (m, 1H), 3.64-4.13 (m, 4H), 4.19-4.47 (m, 1H), 5.12 (d, J=6.1 Hz, 1H), 6.31-6.78 (m, 1H), 6.96-7.15 (m, 2H), 7.17-7.34 (m, 2H), 7.34-7.63 (m, 5H), 7.64-7.81 (m, 2H); LC-MS: (ES) m/z 585.4 (M+H⁺).

### Example S141: Synthesis of (2S,3R,4aS,7aR)-2-(4-(cyclopentylamino)phenyl)-N-(4-(dimethyl-amino)phenyl)-1-(2-fluoro-6-methylbenzoyl)octahydrofuro[3,4-b]pyridine-3-carboxamide (Compound No. 125)

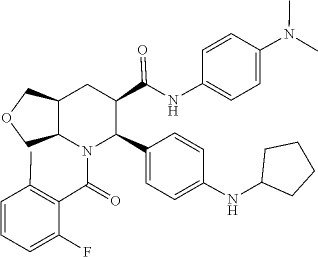 [SMILES: Cc1cccc(F)c1C(=O)N1[C@H](c2ccc(NC3CCCC3)cc2)[C@H](C(=O)Nc2ccc(N(C)C)cc2)C[C@@H]2COC[C@@H]21]

The title compound was synthesized in similar fashion as Examples S135 and S136. ¹H NMR (400 MHz, METHANOL-d₄) δ ppm 1.39 (s, 1H), 1.71 (br s, 4H), 1.85 (br s, 2H), 1.92-2.06 (m, 3H), 2.08-2.50 (m, 3H), 2.51-2.83 (m, 1H), 3.22 (s, 3H), 3.27 (s, 3 H), 3.32-3.52 (m, 1H), 3.67-4.14 (m, 4H), 4.16-4.48 (m, 1H), 4.97-5.35 (m, 1H), 6.30-6.79 (m, 1H), 6.94-7.16 (m, 2H), 7.17-7.30 (m, 2H), 7.30-7.63 (m, 5H), 7.63-7.81 (m, 2H); LC-MS: (ES) m/z 585.4 (M+H⁺).

### Example S142: Synthesis of (2R,3S,4aR,7aS)-2-(4-(cyclopentylamino)phenyl)-1-(2-fluoro-6-methylbenzoyl)-N-(1-methyl-1H-indazol-5-yl)octahydrofuro[3,4-b]pyridine-3-carboxamide (Compound No. 126)

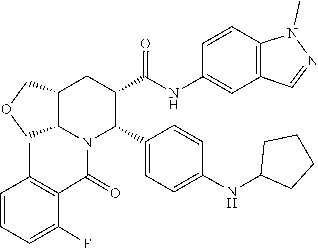

The title compound was synthesized in similar fashion as Examples S135 and S136. ¹H NMR (400 MHz, METHANOL-d₄) δ ppm 1.42-1.50 (m, 3H), 1.57-1.63 (m, 2 H), 1.71 (br d, J=5.52 Hz, 2H), 1.89-2.02 (m, 3H), 2.14-2.21 (m, 1H), 2.29-2.54 (m, 3 H), 3.05-3.18 (m, 1H), 3.70-3.77 (m, 2H), 3.99-4.04 (m, 4H), 4.19-4.35 (m, 1H), 4.91-4.97 (m, 2H), 6.40 (t, J=8.16 Hz, 1H), 6.55-6.59 (m, 2H), 6.72-6.82 (m, 1H), 7.05-7.25 (m, 3H), 7.36-7.45 (m, 3H), 7.79-7.95 (m, 2H). LC-MS: (ES) m/z 596.3 (M+H⁺).

### Example S143: Synthesis of cis-1-(2-fluoro-6-methylbenzoyl)-N-(1-methyl-1H-pyrazol-4-yl)-2-(4-((tetra hydro-2H-pyran-4-yl)amino)phenyl)octahydrofuro[3,4-b]pyridine-3-carboxamide (Compound No. 127)

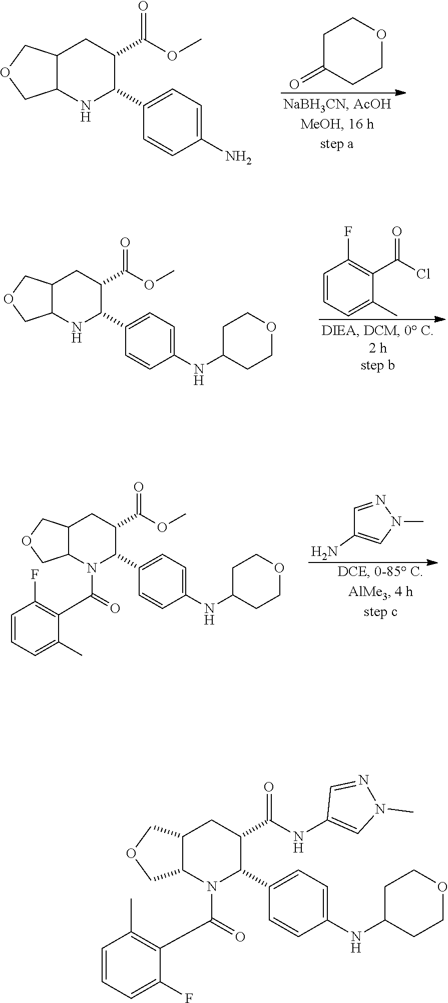

Step a) To a solution of cis-methyl 2-(4-aminophenyl)-1,2,3,4,4a,5,7,7a-octahydrofuro-[3,4-b]pyridine-3-carboxylate (780 mg, 2.82 mmol) and tetrahydropyran-4-one (352.78 mg, 3.52 mmol, 323.65 μL) in MeOH (20 mL) was added CH₃COOH (254.26 mg, 4.23 mmol, 242.15 μL) and NaBH₃CN (886.90 mg, 14.11 mmol) at 0° C. The mixture was stirred at 20° C. for 16 h. After 16 h, NaBH₃CN (200 mg, 3.18 mmol) and CH₃COOH (52.50 mg, 874.24 mol, 50 μL) were added to the reaction mixture, and it was stirred at 20° C. for another 16 h. The reaction mixture was concentrated under reduced pressure to give a residue. The residue was alkalized with aqueous NaHCO₃ (10 mL) and extracted with DCM (50 mL×2). The combined organic layers were dried over anhydrous Na₂SO₄, filtered and concentrated under reduced pressure to give a pure product. The residue was purified by column chromatography (SiO₂, DCM:methanol=100/1) to give cis-methyl 2-[4-(tetrahydropyran-4-ylamino) phenyl]-1,2,3,4,4a,5,7,7a-octahydrofuro[3,4-b]pyridine-3-carboxylate (330 mg, 732.43 mol, 25.95% yield, 80% purity) as a light yellow gum. ¹H NMR (400 MHz, CDCl₃) δ 1.35-1.49 (m, 1H), 1.35-1.49 (m, 1H), 1.95-2.05 (m, 2H), 2.14 (q, J=5.95 Hz, 2H), 2.18-2.27 (m, 1H), 2.83-2.91 (m, 1H), 3.35 (s, 3H), 3.41-3.54 (m, 5H), 3.68-3.78 (m, 2H), 3.86 (t, J=8.31 Hz, 1H), 3.93-4.01 (m, 4H), 6.53 (d, J=8.56 Hz, 2H), 6.60 (d, J=8.31 Hz, 1H), 7.06-7.13 (m, 2H). LC-MS: (ES) m/z 361.2 (M+H⁺).

Step b) To a solution of cis-methyl 2-[4-(tetrahydropyran-4-ylamino)phenyl]-1,2,3,4,4a,5,7,7a-octahydrofuro[3,4-b]pyridine-3-carboxylate (399 mg, 1.11 mmol) in DCM (15 mL) was added DIEA (500.72 mg, 3.87 mmol, 674.83 μL), then the 2-fluoro-6-methyl-benzoyl chloride (200.60 mg, 1.16 mmol) in DCM (5 mL) was added dropwise. The mixture was stirred at 0° C. for 3 h. The reaction mixture was quenched by addition H₂O (10 mL), and then extracted with DCM (30 mL×2). The combined organic layers were dried over anhydrous Na₂SO₄, filtered and concentrated under reduced pressure to give a residue. The residue was purified by column chromatography (SiO₂, DCM:methanol=50/1) to give cis-methyl 1-(2-fluoro-6-methyl-benzoyl)-2-[4-(tetrahydropyran-4-ylamino)phenyl]-3,4,4a,5,7,7a-hexahydro-2H-furo[3,4-b]pyridine-3-carboxylate (510 mg, 821.64 umol, 74.22% yield, 80% purity) as a white solid. ¹H NMR (400 MHz, CDCl₃) δ 1.47 (br d, J=9.54 Hz, 2H), 1.54 (s, 1H), 2.02 (br s, 2H), 2.15-2.39 (m, 5H), 2.84-3.06 (m, 1H), 3.07-3.33 (m, 1H), 3.43-3.58 (m, 5H), 3.61-3.89 (m, 4H), 3.95-4.06 (m, 3H), 6.32-6.42 (m, 1 H), 6.51-6.58 (m, 1H), 6.59-6.67 (m, 1H), 6.72-6.81 (m, 1H), 6.92-7.08 (m, 2H), 7.17-7.27 (m, 2H). LC-MS: (ES) m/z 497.3 (M+H⁺).

Step c) To a solution of 1-methylpyrazol-4-amine (23.47 mg, 241.66 mol, 11.03 L) in DCE (1.5 mL) was added Al(CH₃)₃ (2 M, 140.97 μL), the mixture stirred at 30° C. for 0.5 h. Then the cis-methyl 1-(2-fluoro-6-methyl-benzoyl)-2-[4-(tetrahydropyran-4-ylamino)phenyl]-3, 4,4a,5,7,7a-hexahydro-2H-furo[3,4-b]pyridine-3-carboxylate (40 mg, 80.55 mol) in DCE (1 mL) was added and stirred at 85° C. for 3.5 h. The reaction mixture was diluted with aqueous NaHCO₃ (8 mL) and extracted with DCM (30 mL×2). The combined organic layers were dried over anhydrous Na₂SO₄, filtered and concentrated under reduced pressure to give a residue. The residue was purified by prep-HPLC (HCl condition, column: Phenomenex Gemini-NX 150×30 mm×5 μm; mobile phase: [water (0.05% HCl)-ACN]; B %: 25%-55%, 7 min) to give cis-1-(2-fluoro-6-methyl-benzoyl)-N-(1-methylpyrazol-4-yl)-2-[4-(tetrahydropyran-4-ylamino)phenyl]-3,4,4a,5,7,7a-hexahydro-2H-furo[3,4-b]pyridine-3-carboxamide (12 mg, 20.06 mol, 24.91% yield, 100% purity, HCl) as a yellow solid. ¹H NMR (400 MHz, METHANOL-d₄) δ 1.27-1.41 (m, 2H), 1.69-1.80 (m, 2H), 1.85 (br s, 1H), 1.97-2.10 (m, 2H), 2.14-2.29 (m, 1H), 2.35-2.49 (m, 2H), 2.52-2.84 (m, 1H), 3.12-3.25 (m, 1H), 3.34-3.45 (m, 2H), 3.65-3.76 (m, 2H), 3.80-3.90 (m, 3H), 3.95-4.12 (m, 3H), 4.18-4.44 (m, 1H), 4.99-5.37 (m, 1H), 6.32-6.77 (m, 1H), 6.94-7.36 (m, 5H), 7.37-7.50 (m, 2H), 7.60-8.00 (m, 2H). LC-MS: (ES) m/z 562.3 (M+H⁺).

### Example S144: Synthesis of cis-1-(2-fluoro-6-methylbenzoyl)-N-(pyridin-3-yl)-2-(4-((tetrahydro-2H-pyran-4-yl)amino)phenyl)octahydrofuro[3,4-b]pyridine-3-carboxamide (Compound No. 128)

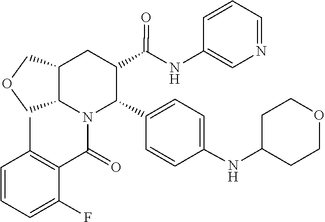

The title compound was synthesized in similar fashion as Example S143. ¹H NMR (400 MHz, METHANOL-d₄) δ 1.28-1.41 (m, 2H), 1.71 (br s, 2H), 1.86 (br s, 2H), 2.00-2.12 (m, 1H), 2.22-2.36 (m, 1H), 2.40-2.51 (m, 2H), 2.51-2.86 (m, 1H), 3.37-3.47 (m, 2H), 3.67-3.91 (m, 3H), 3.97-4.13 (m, 3H), 4.22-4.46 (m, 1H), 4.96-5.36 (m, 1H), 6.42-6.77 (m, 1H), 6.97-7.32 (m, 4H), 7.34-7.46 (m, 2H), 7.79 (br d, J=8.25 Hz, 1H), 7.91-8.12 (m, 1H), 8.31-8.65 (m, 2H), 8.92-9.62 (m, 1H). LC-MS: (ES) m/z 559.3 (M+H⁺).

### Example S145: Synthesis of cis-1-(2-fluoro-6-methylbenzoyl)-N-phenyl-2-(4-((tetrahydro-2H-pyran-4-yl)amino)phenyl)octahydrofuro[3,4-b]pyridine-3-carboxamide (Compound No. 129)

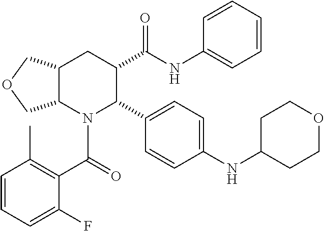

The title compound was synthesized in similar fashion as Example S143. ¹H NMR (400 MHz, METHANOL-d₄) 1H NMR (400 MHz, METHANOL-d₄) δ ppm 1.26-1.40 (m, 2 H), 1.67-1.87 (m, 3H), 1.99-2.05 (m, 1H), 2.12-2.24 (m, 1H), 2.44 (d, J=12.13 Hz, 2 H), 2.51-2.82 (m, 1H), 3.13-3.27 (m, 1H), 3.34-3.44 (m, 2H), 3.50-3.91 (m, 3H), 3.96-4.14 (m, 3H), 4.20-4.46 (m, 1H), 4.94-5.17 (m, 1H), 6.27-6.76 (m, 1H), 6.97-7.16 (m, 3H), 7.18-7.32 (m, 5H), 7.35-7.48 (m, 3H), 7.73-7.83 (m, 1H). LC-MS: (ES) m z 558.3 (M+H⁺).

### Example S146: Synthesis of cis-N-(3-(dimethylphosphoryl)-4-methylphenyl)-1-(2-fluoro-6-methylbenzoyl)-2-(4-((tetrahydro-2H-pyran-4-yl)amino)phenyl)octahydrofuro[3,4-b]pyridine-3-carboxamide (Compound No. 130)

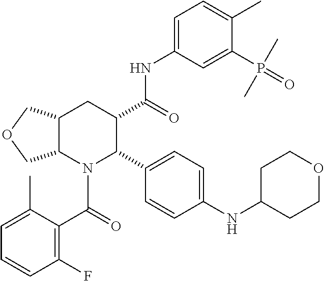

The title compound was synthesized in similar fashion as Example S143. ¹H NMR (400 MHz, METHANOL-d₄) δ 1.27-1.41 (m, 5H), 1.79-1.87 (m, 6H), 1.98-2.09 (m, 2 H), 2.22 (br dd, J=19.44, 7.70 Hz, 1H), 2.44 (d, J=11.49 Hz, 2H), 2.50-2.66 (m, 4H), 2.78 (br s, 1H), 3.22 (br d, J=9.05 Hz, 1H), 3.34-3.48 (m, 3H), 3.66-3.91 (m, 3H), 3.94-4.12 (m, 3H), 4.20-4.34 (m, 1H), 5.08-5.38 (m, 1H), 6.30-6.76 (m, 1H), 7.00-7.17 (m, 3 H), 7.18-7.35 (m, 4H), 7.38-7.48 (m, 1H), 7.53-7.62 (m, 1H), 7.75-7.87 (m, 1 H). LC-MS: (ES) m/z 648.4 (M+H⁺).

### Example S147: Synthesis of cis-N-(benzo[d]oxazol-6-yl)-1-(2-fluoro-6-methylbenzoyl)-2-(4-((tetrahydro-2H-pyran-4-yl)amino)phenyl)octahydrofuro[3,4-b]pyridine-3-carboxamide (Compound No. 131)

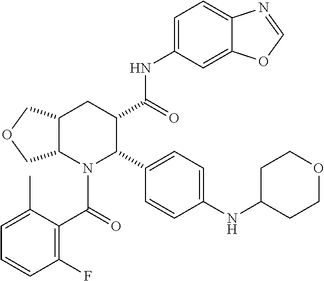

The title compound was synthesized in similar fashion as Example S143. ¹H NMR (400 MHz, METHANOL-d₄) δ 1.36-1.44 (m, 3H), 1.83-2.08 (m, 4H), 2.12-2.26 (m, 2 H), 2.29-2.48 (m, 2H), 3.05-3.25 (m, 1H), 3.41-3.57 (m, 3H), 3.83-4.10 (m, 4H), 4.14-4.33 (m, 1H), 5.16-5.37 (m, 1H), 6.40 (br d, J=7.83 Hz, 1H), 6.47-6.79 (m, 3H), 7.02-7.13 (m, 2H), 7.13-7.30 (m, 1H), 7.32-7.43 (m, 2H), 7.53-7.68 (m, 2H), 8.36-8.42 (m, 1H). LC-MS: (ES) m/z 599.3 (M+H⁺).

### Example S148: Synthesis of cis-N-(3-cyano-4-methylphenyl)-1-(2-fluoro-6-methylbenzoyl)-2-(4-((tetrahydro-2H-pyran-4-yl)amino)phenyl)octahydrofuro[3,4-b]pyridine-3-carboxamide (Compound No. 132)

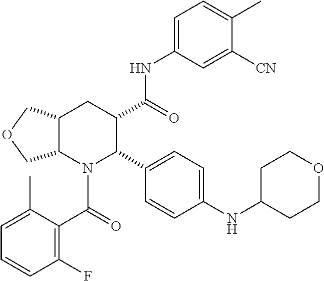

The title compound was synthesized in similar fashion as Example S143. ¹H NMR (400 MHz, METHANOL-d₄) δ1.31 (br d, J=15.41 Hz, 1H), 1.58 (s, 1H), 1.61-1.75 (m, 2 H), 1.76-1.95 (m, 3H), 2.06 (br s, 1H), 2.28-2.53 (m, 5H), 2.64-2.92 (m, 1H), 3.34-3.48 (m, 2H), 3.61-3.91 (m, 3H), 3.93-4.11 (m, 3H), 4.47-4.78 (m, 1H), 5.21-5.31 (m, 1H), 6.49-6.70 (m, 1H), 7.01-7.14 (m, 3H), 7.15-7.37 (m, 4H), 7.45 (br dd, J=16.51, 8.19 Hz, 1H), 7.52-7.69 (m, 1H), 7.77-7.92 (m, 1H). LC-MS: (ES) m/z 597.4 (M+H⁺).

### Example S149: Synthesis of cis-(2R,3S,4aR,7aS)-1-(2-fluoro-6-methylbenzoyl)-N-(2-methyl-pyrimidin-5-yl)-2-(4-((tetrahydro-2H-pyran-4-yl)amino)phenyl)octahydrofuro[3,4-b]pyridine-3-carboxamide (Compound No. 133)

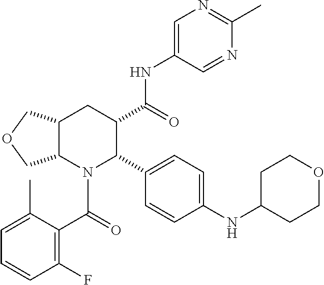

The title compound was synthesized in similar fashion as Example S143. ¹H NMR (400 MHz, METHANOL-d₄) δ 1.25-1.42 (m, 2H), 1.66-1.95 (m, 2H), 1.97-2.06 (m, 1 H), 2.14-2.31 (m, 1H), 2.34-2.62 (m, 3H), 2.66-2.83 (m, 3H), 3.35-3.50 (m, 3H), 3.71-3.91 (m, 2H), 3.95-4.14 (m, 3H), 4.19-4.34 (m, 1H), 5.03-5.19 (m, 1H), 6.38-6.78 (m, 1H), 7.01-7.16 (m, 2H), 7.17-7.34 (m, 2H), 7.35-7.53 (m, 1H), 7.80 (br d, J=6.85 Hz, 1H), 8.83 (br s, 1H), 9.02-9.15 (m, 1H). LC-MS: (ES) m/z 574.4 (M+H⁺).

### Example S150: Synthesis of cis-1-(2-fluoro-6-methylbenzoyl)-N-(1-methyl-1H-indazol-5-yl)-2-(4-((tetrahydro-2H-pyran-4-yl)amino)phenyl)octahydrofuro[3,4-b]pyridine-3-carboxamide (Compound No. 134)

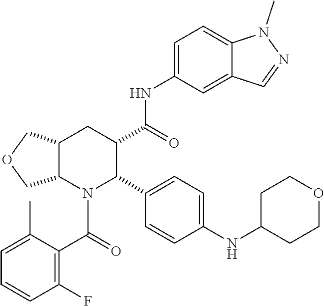

The title compound was synthesized in similar fashion as Example S143. ¹H NMR (400 MHz, METHANOL-d₄) δ 1.25-1.42 (m, 2H), 1.56-1.96 (m, 6H), 1.99-2.27 (m, 2 H), 2.29-2.54 (m, 2H), 2.62-3.10 (m, 1H), 3.56-3.92 (m, 4H), 3.93-4.12 (m, 6H), 5.13-5.38 (m, 1H), 6.45-6.76 (m, 1H), 7.01-7.20 (m, 3H), 7.20-7.31 (m, 2H), 7.31-7.38 (m, 1H), 7.39-7.53 (m, 2H), 7.60-7.99 (m, 3H). LC-MS: (ES) m/z 612.4 (M+H⁺).

### Example S151: Synthesis of cis-1-(2-fluoro-6-methylbenzoyl)-N-(4-methyl-3-(trifluoromethyl)-phenyl)-2-(4-((tetrahydro-2H-pyran-4-yl)amino)phenyl)octahydrofuro[3,4-b]pyridine-3-carboxamide (Compound No. 135)

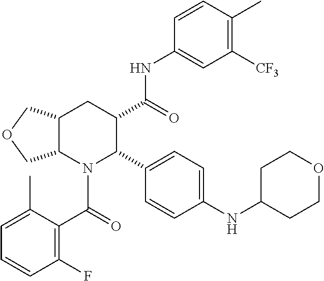

The title compound was synthesized in similar fashion as Example S143. ¹H NMR (400 MHz, METHANOL-d₄) δ1.27-1.34 (m, 2H), 1.59-1.75 (m, 2H), 1.76-1.92 (m, 2 H), 1.99-2.10 (m, 2H), 2.36-2.46 (m, 5H), 3.11-3.27 (m, 1H), 3.34-3.44 (m, 3H), 3.62-3.91 (m, 3H), 3.93-4.13 (m, 3H), 4.20-4.34 (m, 1H), 5.05-5.38 (m, 1H), 6.27-6.77 (m, 1H), 6.89-7.26 (m, 5H), 7.26-7.43 (m, 3H), 7.43-7.63 (m, 1H), 7.70-7.88 (m, 1 H). LC-MS: (ES) m/z 640.3 (M+H⁺).

### Example S152: Synthesis of cis-N-(4-(dimethylamino)phenyl)-1-(2-fluoro-6-methylbenzoyl)-2-(4-((tetrahydro-2H-pyran-4-yl)amino)phenyl)octahydrofuro[3,4-b]pyridine-3-carboxamide (Compound No. 136)

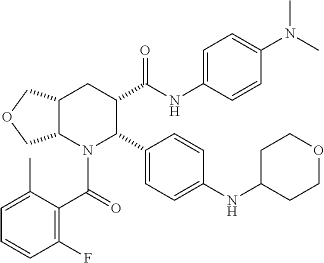

The title compound was synthesized in similar fashion as Example S143. ¹H NMR (400 MHz, METHANOL-d₄) δ1.26-1.43 (m, 2H), 1.57-1.77 (m, 2H), 1.79-1.95 (m, 2 H), 1.97-2.32 (m, 3H), 2.38-2.47 (m, 2H), 2.49-2.82 (m, 1H), 3.22 (s, 3H), 3.26 (s, 3 H), 3.36-3.47 (m, 2H), 3.59-3.92 (m, 2H), 3.95-4.13 (m, 3H), 4.19-4.33 (m, 1H), 4.89-5.11 (m, 1H), 6.29-6.78 (m, 1H), 6.85-7.00 (m, 1H), 7.01-7.16 (m, 2H), 7.16-7.24 (m, 1H), 7.25-7.50 (m, 4H), 7.53-7.60 (m, 1H), 7.65-7.78 (m, 2H), 9.89-10.02 (m, 0.2 H), 10.40 (s, 0.3 H). LC-MS: (ES) m/z 601.3 (M+H⁺).

### Example S153: Synthesis of (2R,3S,4aR,7aS)-2-(4-(cyclopentylamino)phenyl)-1-(2-fluoro-6-methylbenzoyl)-N-(1-(2-hydroxyethyl)-1H-indazol-5-yl)octahydrofuro[3,4-b]pyridine-3-carboxamide (Compound No. 137)

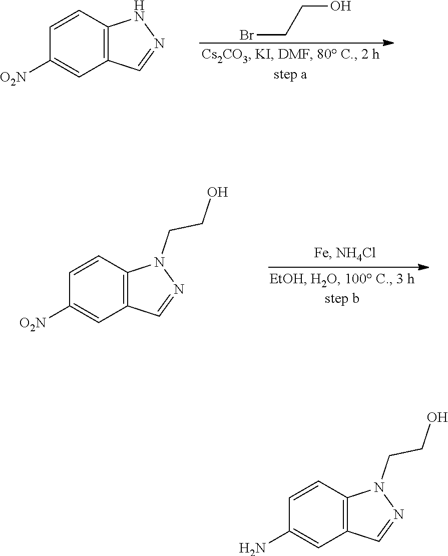

Step a) To a solution of 5-nitro-1H-indazole (1.4 g, 8.58 mmol) and 2-bromoethanol (1.39 g, 11.16 mmol, 792.14 μL) in DMF (15 mL) was added Cs₂CO₃ (5.59 g, 17.16 mmol), KI (142.46 mg, 858.19 μmol) at 20° C. under N₂. The mixture was stirred at 80° C. for 2 h. The reaction mixture was concentrated under reduced pressure to give a residue. The residue was purified by column chromatography (SiO₂, petroleum ether/ethyl acetate=100/1 to 0/1 Plate 1). The compound 2-(5-nitroindazol-1-yl)ethanol (1 g, 4.83 mmol, 56.24% yield) was obtained as a yellow solid. ¹H NMR (400 MHz, CDCl₃) δ ppm 2.58 (t, J=5.82 Hz, 1H), 4.13-4.26 (m, 2H), 4.46-4.64 (m, 2H), 7.56 (d, J=9.26 Hz, 1H), 8.26 (s, 1H), 8.31 (dd, J=9.26, 2.00 Hz, 1H), 8.76 (d, J=2.00 Hz, 1H). LC-MS: (ES) m/z 208.1 (M+H⁺).

Step b) To a solution of 2-(5-nitroindazol-1-yl)ethanol (0.9 g, 4.34 mmol), iron (1.94 g, 34.75 mmol) and NH₄Cl (116.18 mg, 2.17 mmol) in EtOH (20 mL) and H₂O (4 mL) was degassed and purged with N₂ 3 times, and then the mixture was stirred at 100° C. for 3 h under a N₂ atmosphere. The reaction mixture was filtered and the filtrate was concentrated under reduced pressure to give a residue. The residue was purified by flash silica gel chromatography (ISCO®; 12 g SepaFlash® Silica Flash Column, eluent of 0˜3% DCM:methanol® 30 mL/min). The compound 2-(5-aminoindazol-1-yl)ethanol (700 mg, 3.95 mmol, 90.94% yield, 100% purity) was obtained as a light yellow solid. ¹H NMR (400 MHz, CDCl₃) δ ppm 3.56 (br s, 2H), 4.07-4.14 (m, 2H), 4.40-4.46 (m, 2H), 6.91 (dd, J=8.78, 2.01 Hz, 1H), 6.96 (d, J=1.76 Hz, 1H), 7.29 (s, 1H), 7.83 (s, 1H). LC-MS: (ES) m/z 178.1 (M+H⁺).

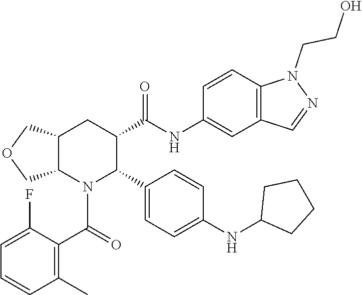 [SMILES: Cc1cccc(F)c1C(=O)N1[C@@H]2COC[C@@H]2C[C@H](C(=O)Nc2ccc3c(cnn3CCO)c2)[C@@H]1c1ccc(NC2CCCC2)cc1]

The title compound was synthesized in similar fashion as Example S143. ¹H NMR (400 MHz, METHANOL-d₄) δ ppm 1.68 (br s, 4H), 1.82 (br s, 2H), 1.94 (br s, 2H), 2.02-2.17 (m, 2H), 2.32-2.51 (m, 2H), 2.55-2.85 (m, 1H), 3.26 (br d, J=9.03 Hz, 2H), 3.33-3.40 (m, 1H), 3.70-3.83 (m, 1H), 3.86-3.99 (m, 4H), 4.00-4.12 (m, 1H), 4.24-4.52 (m, 3H), 6.45-6.76 (m, 1H), 7.02-7.18 (m, 3H), 7.21-7.31 (m, 2H), 7.33-7.53 (m, 3H), 7.54-7.66 (m, 1H), 7.82-7.97 (m, 2H). LC-MS: (ES) m/z 626.3 (M+H).

### Example S154: Synthesis of (2R,3S,4aR,7aS)-2-(4-(cyclopentylamino)phenyl)-1-(2-fluoro-6-methylbenzoyl)-N-(JH-indazol-5-yl)octahydrofuro[3,4-b]pyridine-3-carboxamide (Compound No. 138)

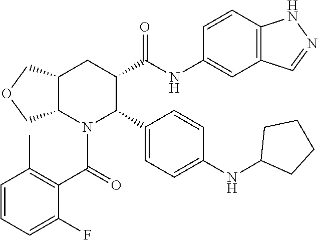 [SMILES: Cc1cccc(F)c1C(=O)N1[C@@H]2COC[C@@H]2C[C@H](C(=O)Nc2ccc3[nH]ncc3c2)[C@@H]1c1ccc(NC2CCCC2)cc1]

The title compound was synthesized in similar fashion as Example S143. ¹H NMR (400 MHz, METHANOL-d₄) δ ppm 1.67 (br d, J=3.76 Hz, 4H), 1.82 (br s, 2H), 1.96 (s, 2 H), 2.08-2.32 (m, 2H), 2.45 (d, J=12.05 Hz, 2H), 2.65-2.84 (m, 1H), 3.23 (br s, 2H), 3.32-3.37 (m, 2H), 3.67-3.96 (m, 3H), 4.03-4.34 (m, 1H), 5.18 (br d, J=6.27 Hz, 1H), 6.46-6.77 (m, 1H), 7.07-7.16 (m, 2H), 7.27 (dd, J=8.41, 1.88 Hz, 2H), 7.41-7.45 (m, 2 H), 7.65 (br d, J=2.51 Hz, 1H), 7.82 (d, J=8.78 Hz, 1H), 7.92-8.00 (m, 2H). LC-MS: (ES) m z 582.3 (M+H⁺).

### Example S155: Synthesis of (2R,3S,4aR,7aS)-2-(4-(cyclopentylamino)phenyl)-1-(2-fluoro-6-methylbenzoyl)-N-(1-methyl-1H-indol-5-yl)octahydrofuro[3,4-b]pyridine-3-carboxamide (Compound No. 139)

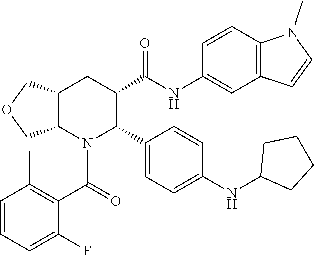 [SMILES: Cc1cccc(F)c1C(=O)N1[C@@H]2COC[C@@H]2C[C@H](C(=O)Nc2ccc3c(ccn3C)c2)[C@@H]1c1ccc(NC2CCCC2)cc1]

The title compound was synthesized in similar fashion as Example S143. ¹H NMR (400 MHz, METHANOL-d₄) δ ppm 1.64-1.72 (m, 4H), 1.81 (br s, 2H), 1.95-2.06 (m, 3 H), 2.14-2.29 (m, 1H), 2.42-2.45 (m, 1H), 2.60 (br d, J=17.88 Hz, 1H), 3.13-3.25 (m, 2 H), 3.35-3.49 (m, 1H), 3.73-3.81 (m, 4H), 3.89-4.11 (m, 3H), 4.22-4.30 (m, 1H), 6.28-6.49 (m, 1H), 6.71-6.97 (m, 2H), 7.03-7.16 (m, 4H), 7.19-7.32 (m, 4H), 7.39-7.42 (m, 1H), 7.73-7.84 (m, 1H). LC-MS: (ES) m/z 595.3 (M+H⁺).

### Example S156: Synthesis of (2S,3R,4aS,7aR)-2-(4-(cyclopentylamino)phenyl)-1-(2-fluoro-6-methylbenzoyl)-N-(1-methyl-1H-indazol-5-yl)octahydrofuro[3,4-b]pyridine-3-carboxamide (Compound No. 140)

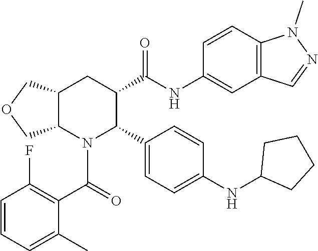 [SMILES: Cc1cccc(F)c1C(=O)N1[C@@H]2COC[C@@H]2C[C@H](C(=O)Nc2ccc3c(cnn3C)c2)[C@@H]1c1ccc(NC2CCCC2)cc1]

The title compound was synthesized in similar fashion as Example S143. ¹H NMR (400 MHz, METHANOL-d₄) δ ppm 1.69 (br s, 4H), 1.84 (br s, 2H), 1.99-2.32 (m, 4H), 2.47 (d, J=11.80 Hz, 2H), 2.64-2.90 (m, 1H), 3.14-3.31 (m, 2H), 3.38-3.61 (m, 1H), 3.90 (br dd, J=9.03, 4.77 Hz, 1H), 4.02-4.08 (m, 4H), 4.09-4.21 (m, 1H), 4.26-4.46 (m, 1H), 4.98-5.27 (m, 1H), 6.32-6.80 (m, 1H), 7.04-7.24 (m, 4H), 7.26-7.36 (m, 2H), 7.38-7.68 (m, 4H), 7.79-7.98 (m, 3H). LC-MS: (ES) m/z 596.3 (M+H⁺).

### Example S157: Synthesis of cis-2-(4-((3,3-dimethylmorpholino)methyl)phenyl)-1-(2-fluoro-6-methylbenzoyl)-N-(4-methyl-3-(trifluoromethyl)phenyl)octahydrofuro[3,4-b]pyridine-3-carboxamide (Compound No. 141)

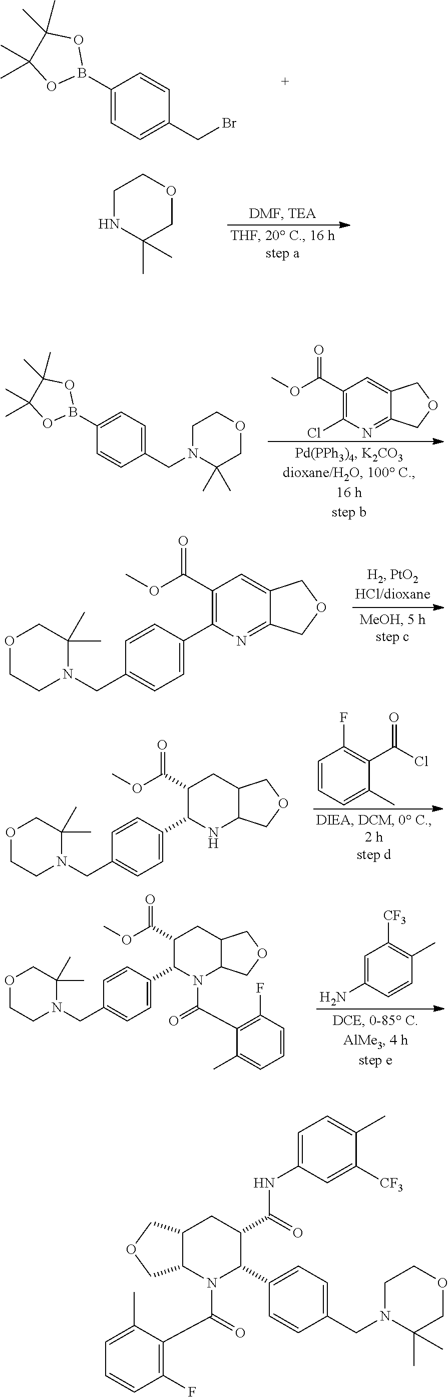

Step a) A mixture of 2-[4-(bromomethyl)phenyl]-4,4,5,5-tetramethyl-1,3,2-dioxaborolane (0.9 g, 3.03 mmol), 3,3-dimethylmorpholine (523.52 mg, 4.55 mmol) and TEA (1.23 g, 12.12 mmol, 1.69 mL) in THF (12 mL) and DMF (1 mL) was degassed and purged with N₂ 3 times, and then the mixture was stirred at 20° C. for 16 h under a N₂ atmosphere. The reaction mixture was concentrated under reduced pressure to remove THF. The residue was diluted with brine (10 mL) and extracted with ethyl acetate (50 mL). The combined organic layers was dried over anhydrous Na₂SO₄, filtered and concentrated under reduced pressure to give a residue. The residue was purified by column chromatography (SiO₂, petroleum ether/ethyl acetate=5/1) to give 3,3-dimethyl-4-[[4-(4,4,5,5-tetra methyl-1,3,2-dioxaborolan-2-yl)phenyl]methyl]morpholine (585 mg, 1.68 mmol, 55.36% yield, 95% purity) as a white solid. ¹H NMR (400 MHz, CDCl₃) δ 1.12 (s, 6H), 1.35 (s, 12H), 2.35-2.42 (m, 2H), 3.40 (s, 2H), 3.53 (br s, 2H), 3.60-3.65 (m, 2H), 7.36 (d, J=7.83 Hz, 2H), 7.76 (d, J=7.83 Hz, 2H). LC-MS: (ES) m/z 332.3 (M+H⁺).

Step b) A mixture of methyl 2-chloro-5,7-dihydrofuro[3,4-b]pyridine-3-carboxylate (400 mg, 1.87 mmol), 3,3-dimethyl-4-[[4-(4,4,5,5-tetramethyl-1,3,2-dioxaborolan-2-yl)phenyl]methyl] morpholine (744.34 mg, 2.25 mmol), Pd(PPh₃)₄ (216.38 mg, 187.25 mol) and K₂CO₃ (2 M, 3.28 mL) in dioxane (10 mL) was degassed and purged with N₂ 3 times, and then the mixture was stirred at 100° C. for 16 h under a N₂ atmosphere. The reaction mixture was concentrated under reduced pressure to remove dioxane and then extracted with EtOAc 160 mL (80 mL×2). The combined organic layers were washed with brine (20 mL), dried over anhydrous Na₂S04, filtered and concentrated under reduced pressure to give a residue. The residue was purified by flash silica gel chromatography (ISCO®; 20 g SepaFlash® Silica Flash Column, eluent of 0-30% ethyl acetate/petroleum ether gradient@35 mL/min) to give compound methyl 2-[4-[(3,3-dimethylmorpholin-4-yl)methyl]phenyl]-5,7-dihydrofuro[3,4-b]pyridine-3-carboxylate (860 mg, 1.80 mmol, 96.07% yield, 80% purity) as a light yellow gum. ¹H NMR (400 MHz, CDCl₃) δ 1.14 (s, 6 H), 2.37-2.44 (m, 2H), 3.38-3.43 (m, 2H), 3.58 (br s, 2H), 3.61-3.67 (m, 2H), 3.71 (s, 3 H), 5.14 (t, J=1.63 Hz, 2H), 5.24 (s, 2H), 7.41-7.48 (m, 4H), 7.97 (s, 1H). LC-MS: (ES) m z 383.2 (M+H⁺).

Step c) To a solution of methyl 2-[4-[(3,3-dimethylmorpholin-4-yl)methyl]phenyl]-5,7-dihydrofuro[3,4-b]pyridine-3-carboxylate (0.8 g, 1.78 mmol) and HCl/dioxane (4 M, 889.00 μL) in MeOH (15 mL) was added PtO₂ (121.12 mg, 533.40 mol). Then the mixture was degassed and purged with H₂ (15 psi) 3 times, and then the mixture was stirred at 20° C. for 3 h under H₂ atmosphere. The reaction mixture was filtered and concentrated under reduced pressure to give a residue. The residue was alkalized with aqueous NaHCO₃ 10 mL and extracted with DCM 100 mL (50 mL×2). The combined organic layers were dried over anhydrous Na₂SO₄, filtered and concentrated under reduced pressure to give a crude product. The residue was purified by column chromatography (SiO₂, petroleum ether/ethyl acetate=100/1 to 10/1, plate 2) to give cis-methyl 2-[4-[(3,3-dimethylmorpholin-4-yl)methyl]phenyl]-1,2,3,4,4a,5,7,7a-octahydrofuro[3,4-b]pyridine-3-carboxylate (430 mg, 996.14 mol, 56.03% yield, 90% purity) as a light yellow gum. ¹H NMR (400 MHz, METHANOL-d₄) δ 1.13 (s, 6H), 2.14 (dt, J=14.07, 4.28 Hz, 1H), 2.25-2.36 (m, 2H), 2.37-2.44 (m, 2H), 2.91-2.99 (m, 1H), 3.32-3.33 (m, 3H), 3.37 (s, 2H), 3.48-3.54 (m, 3H), 3.58-3.63 (m, 2H), 3.73 (t, J=8.50 Hz, 1H), 3.78 (dd, J=9.51, 1.50 Hz, 1H), 3.83-3.89 (m, 1H), 3.96 (dd, J=9.51, 5.13 Hz, 1H), 4.05 (d, J=5.13 Hz, 1H), 7.24-7.32 (m, 4H). LC-MS: (ES) m/z 389.4 (M+H⁺).

Step d) To a solution of cis-methyl-2-[4-[(3,3-dimethylmorpholin-4-yl)methyl]phenyl]-1,2, 3,4,4a,5,7,7a-octahydrofuro[3,4-b]pyridine-3-carboxylate (400 mg, 1.03 mmol) in DCM (15 mL) was added DIEA (399.20 mg, 3.09 mmol, 538.00 μL), then the 2-fluoro-6-methyl-benzoyl chloride (213.23 mg, 1.24 mmol) in DCM (2 mL) was added at 0° C. The mixture was stirred at 0° C. for 2 h. The reaction mixture was filtered and concentrated under reduced pressure to give a residue. The residue was purified by flash silica gel chromatography (ISCO®; 12 g SepaFlash® Silica Flash Column, eluent of 0˜3% ethyl acetate/petroleum ether gradient@30 mL/min) to give cis-methyl 2-[4-[(3,3-dimethylmorpholin-4-yl)methyl]phenyl]-1-(2-fluoro-6-methyl-benzoyl)-3,4,4a,5,7,7a-hexahydro-2H-furo[3,4-b]pyridine-3-carboxylate (280 mg, 97% purity) as a light yellow gum. ¹H NMR (400 MHz, DMSO-d₆) δ 0.95-1.08 (m, 6H), 1.24 (s, 1H), 1.73-1.96 (m, 1 H), 2.18-2.38 (m, 4H), 2.58-2.86 (m, 1H), 3.00-3.10 (m, 1H), 3.11-3.22 (m, 1H), 3.27 (br d, J=7.38 Hz, 2H), 3.30-3.33 (m, 3H), 3.36-3.40 (m, 1H), 3.41-3.55 (m, 4H), 3.57-3.68 (m, 2H), 3.69-3.90 (m, 1H), 4.70-4.94 (m, 1H), 6.40-6.79 (m, 2H), 7.06-7.21 (m, 3H), 7.22-7.43 (m, 2H) LC-MS: (ES) m/z 525.3 (M+H⁺).

Step e) To a solution of 4-methyl-3-(trifluoromethyl)aniline (40.06 mg, 228.74 mol, 32.84 μL) in DCE (1.5 mL) was added Al(CH₃)₃ (in toulene) (2 M, 133.43 μL) at 0° C., after 20 min, the cis-methyl2-[4-[(3,3-dimethylmorpholin-4-yl)methyl]phenyl]-1-(2-fluoro-6-methyl-b enzoyl)-3,4,4a,5,7,7a-hexahydro-2H-furo[3,4-b]pyridine-3-carboxylate (40 mg, 76.25 mol) in DCE (1 mL) was added. The mixture was stirred at 85° C. for 3 h 40 min. The reaction mixture was diluted with aqueous NaHCO₃ 8 mL and extracted with DCM (30 mL×2). The combined organic layers were dried over anhydrous Na₂SO₄, filtered and concentrated under reduced pressure to give a residue. The residue was purified by prep-HPLC (HCl condition; column: Phenomenex Gemini-NX 150×30 mm×5 μm; mobile phase: [water (0.05% HCl)-ACN]; B %: 25%-55%, 7 min) to give cis-2-[4-[(3,3-dimethylmorpholin-4-yl)methyl]phenyl]-1-(2-fluoro-6-methyl-benzoyl)-N-[4-methyl-3-(trifluoromethyl)phenyl]-3,4,4a,5,7,7a-hexahydro-2H-furo[3,4-b]pyridine-3-carboxamide (30 mg, 44.93 umol, 58.93% yield, 100% purity) as a light yellow solid. ¹H NMR (400 MHz, METHANOL-d₄) δ 1.26-1.37 (m, 2H), 1.45-1.51 (m, 3H), 1.54-1.57 (m, 3H), 1.99-2.10 (m, 1H), 2.14-2.30 (m, 1H), 2.38 (s, 1H), 2.40-2.47 (m, 3H), 2.51-2.83 (m, 1H), 2.98-3.27 (m, 2H), 3.33-3.47 (m, 1H), 3.55-3.65 (m, 2H), 3.69-3.80 (m, 2H), 3.83-4.14 (m, 4H), 4.20-4.47 (m, 1H), 4.59-4.72 (m, 1H), 4.93-5.13 (m, 1H), 6.31-6.76 (m, 1H), 6.91-6.99 (m, 1H), 7.01-7.16 (m, 1H), 7.17-7.34 (m, 3H), 7.35-7.42 (m, 1H), 7.48 (br d, J=6.78 Hz, 1H), 7.53-7.92 (m, 3H). LC-MS: (ES) m/z 668.4 (M+H⁺).

### Example S158: Synthesis of (2R,3S,4aR,7aS)-2-(4-((3,3-dimethylmorpholino)methyl)phenyl)-1-(2-fluoro-6-methylbenzoyl)-N-(1-methyl-1H-indazol-5-yl)octahydrofuro[3,4-b]pyridine-3-carboxamide (Compound No. 142)

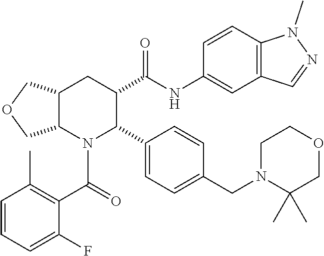

The title compound was synthesized in similar fashion as Example S143. ¹H NMR (400 MHz, METHANOL-d₄) δ ppm 1.31-1.40 (m, 1H), 1.43-1.57 (m, 6H), 1.97-2.34 (m, 3H), 2.41-2.48 (m, 1H), 2.54-2.84 (m, 1H), 2.88-3.15 (m, 1H), 3.18-3.28 (m, 1 H), 3.36-3.66 (m, 3H), 3.68-3.80 (m, 2H), 3.81-3.91 (m, 1H), 3.94-4.06 (m, 5H), 4.07-4.32 (m, 2H), 4.58-4.72 (m, 1H), 4.93-5.18 (m, 1H), 6.32-6.77 (m, 1H), 6.96-7.26 (m, 3H), 7.28-7.55 (m, 5H), 7.63-7.99 (m, 3H). LC-MS: (ES) m/z 640.3 (M+H⁺).

### Example S159: Synthesis cis-2-(4-(cyclopentylamino)phenyl)-1-(2-fluoro-6-methylbenzoyl)-6-methyl-N-(4-methyl-3-(trifluoromethyl)phenyl)octahydro-1H-pyrrolo[3,4-b]pyridine-3-carboxamide (Compound No. 143)

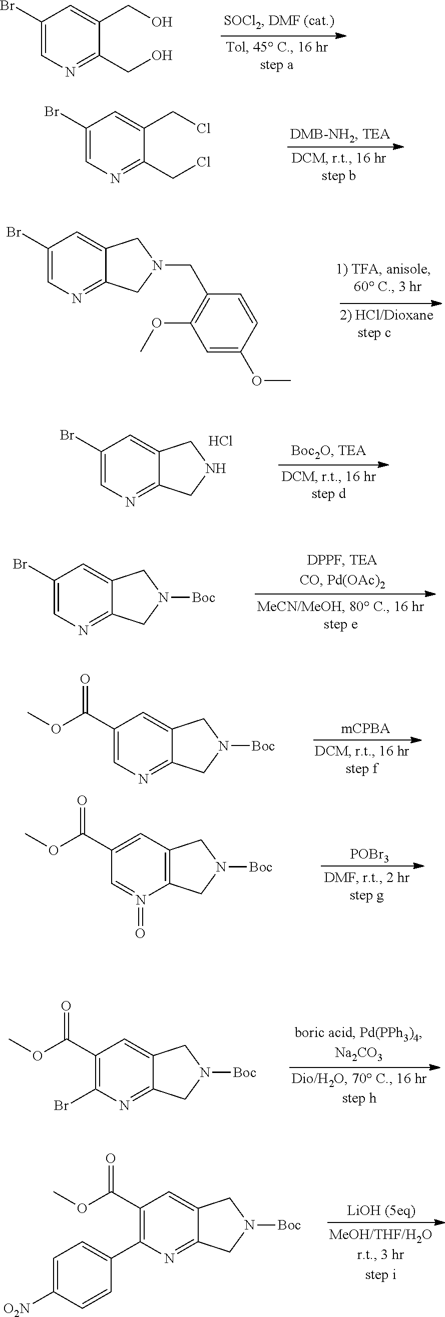

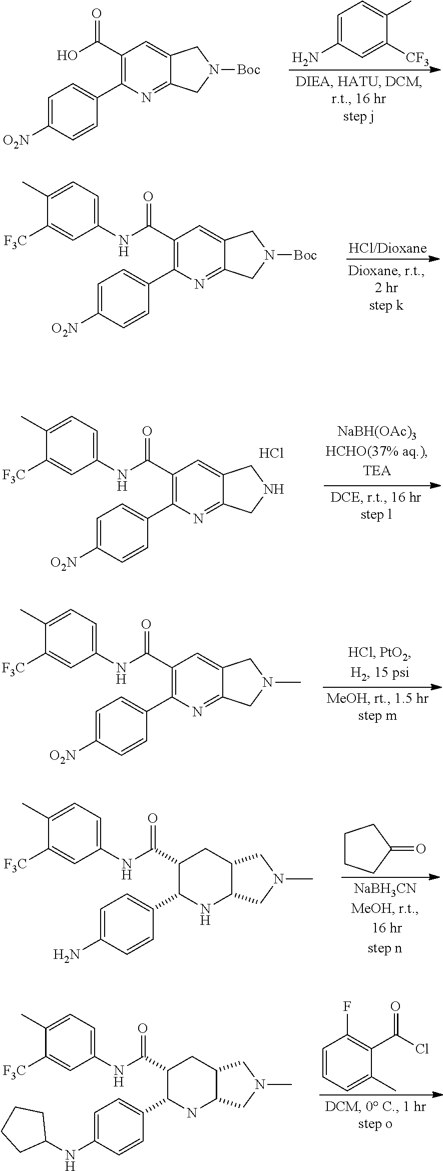

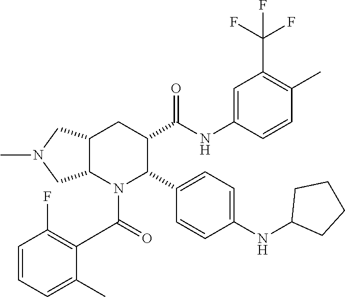

Step a) To a mixture of [5-bromo-2-(hydroxymethyl)-3-pyridyl]methanol (10 g, 45.86 mmol) in toluene (120 mL) was added DMF (670.44 mg, 9.17 mmol, 705.73 μL) and SOCl₂ (43.65 g, 366.89 mmol, 26.62 mL) at 15° C. Then the mixture was stirred at 45° C. for 3 h. The mixture was concentrated under vacuum to give the residue. The residue was dissolved with EtOAc (100 mL)/H₂O (50 mL). The mixture was extracted with EtOAc (2×50 mL). The combined organic layers were washed with saturated NaHCO₃ (3×50 mL), brine (2×30 mL), dried, filtered and concentrated in vacuo to give the desired compound 5-bromo-2,3-bis(chloromethyl)pyridine (12 g, 44.72 mmol, 97.50% yield, 95% purity) as brown oil. ¹H NMR (400 MHz, CDCl₃) δ 4.71 (s, 2H), 4.77 (s, 2H), 7.93 (d, J=1.96 Hz, 1 H), 8.61 (d, J=2.20 Hz, 1H). LC-MS: (ES) m/z 253.9 (M+H⁺).

Step b) To a solution of 5-bromo-2,3-bis(chloromethyl)pyridine (12 g, 44.72 mmol) and (2,4-dimethoxyphenyl)methanamine (7.85 g, 46.95 mmol, 7.07 mL) in DCM (150 mL) was added DIEA (18.55 g, 143.53 mmol, 25 mL) at 0° C. Then the mixture was stirred at 25° C. for 16 h. The mixture was diluted with DCM (50 mL) and washed with brine (2×50 mL), dried, filtered and concentrated in vacuo to give the residue. The residue was purified by flash silica gel chromatography (ISCO®; 120 g SepaFlash® Silica Flash Column, eluent of 0˜40% ethyl acetate/petroleum ether gradient@85 mL/min) to give 3-bromo-6-[(2,4-dimethoxy phenyl)methyl]-5,7-dihydropyrrolo[3,4-b]pyridine (11 g, 29.92 mmol, 66.92% yield, 95% purity) as light brown gum. ¹H NMR (400 MHz, CDCl₃) δ 3.83 (d, J=4.27 Hz, 6 H), 3.89 (s, 2H), 3.97 (s, 4H), 6.49 (dq, J=4.42, 2.29 Hz, 2H), 7.23-7.27 (m, 1H), 7.58 (d, J=2.01 Hz, 1H), 8.43 (d, J=2.01 Hz, 1H). LC-MS: (ES) m/z 351.1 (M+H⁺).

Step c) To a mixture of 3-bromo-6-[(2,4-dimethoxyphenyl)methyl]-5,7-dihydropyrrolo [3,4-b]pyridine (11 g, 29.92 mmol) in TFA (95.29 g, 835.71 mmol, 61.88 mL) was added anisole (20.52 g, 189.77 mmol, 20.63 mL). Then the mixture was stirred at 60° C. for 2 h. The mixture was concentrated in vacuo to give the residue. HCl/dioxane (4 M, 80 mL) was added the residue and the mixture was stirred at 20° C. for 0.5 h. Then the mixture was concentrated to give the residue. The residue was triturated with EtOAc (50 mL) for 15 min at 20° C. The suspension was filtered and the filter cake was dried to give the desired compound 3-bromo-6,7-dihydro-5H-pyrrolo[3,4-b]pyridine (7.5 g, 27.03 mmol, 90.31% yield, 98% purity, 2HCl) as light orange solid. ¹H NMR (400 MHz, METHANOL-d₄) δ 4.57 (s, 2H), 4.72 (s, 2H), 8.10 (s, 1H), 8.66 (s, 1H). LC-MS: (ES) m/z 199.1 (M+H⁺).

Step d) To a solution of 3-bromo-6,7-dihydro-5H-pyrrolo[3,4-b]pyridine (7.5 g, 27.03 mmol, 2HCl) in DCM (80 mL) was added TEA (13.67 g, 135.13 mmol, 18.81 mL) and Boc₂O (8.85 g, 40.54 mmol, 9.31 mL) at 0° C. Then the mixture was stirred at 20° C. for 12 h. The mixture was filtered and the filter cake was eluted with DCM (2×20 mL). The filtrate was washed with brine (3×30 mL), dried, filtered and concentrated in vacuo to give the residue. The residue was purified by flash silica gel chromatography (ISCO®; 40 g SepaFlash® Silica Flash Column, Eluent of 0˜8% ethyl acetate/petroleum ether gradient@40 mL/min) to give tert-butyl 3-bromo-5,7-dihydropyrrolo[3,4-b]pyridine-6-carboxylate (7.8 g, 25.55 mmol, 94.55% yield, 98% purity) as white solid. ¹H NMR (400 MHz, CDCl₃) δ 1.52 (s, 9H), 4.55-4.74 (m, 4H), 7.63-7.77 (m, 1H), 8.54 (br s, 1H). LC-MS: (ES) m/z 299.1 (M+H⁺).

Step e) A mixture of tert-butyl-3-bromo-5,7-dihydropyrrolo[3,4-b]pyridine-6-carboxylate (7.8 g, 25.55 mmol), Pd(OAc)₂ (573.65 mg, 2.56 mmol), DPPF (2.83 g, 5.11 mmol) and TEA (7.76 g, 76.65 mmol, 10.67 mL) in MeCN (80 mL)/MeOH (80 mL) was stirred at 80° C. for 16 h under CO (50 psi). The mixture was diluted with EtOAc (200 mL) and filtered. The filtrate was washed with brine (3×50 mL), dried, filtered and concentrated in vacuo to give the crude product. The crude product was purified by flash silica gel chromatography (ISCO®; 40 g SepaFlash® Silica Flash Column, eluent of 0˜30% ethyl acetate/petroleum ether gradient@40 mL/min) to give 6-tert-butyl-3-methyl 5,7-dihydropyrrolo[3,4-b] pyridine-3,6-dicarboxylate (6.6 g, 23.72 mmol, 92.81% yield, 100% purity) was obtained as off-white solid. ¹H NMR (400 MHz, DMSO-d₆) δ 1.46 (s, 9H), 3.89 (s, 3H), 4.53-4.72 (m, 4H), 8.26 (br d, J=8.07 Hz, 1H), 8.96 (s, 1H). LC-MS: (ES) m z 279.1 (M+H⁺).

Step f) To a solution of 6-tert-butyl-3-methyl5,7-dihydropyrrolo[3,4-b]pyridine-3,6-dicarboxylate (6 g, 21.56 mmol) in DCM (120 mL) was added m-CPBA (9.30 g, 43.12 mmol, 80% purity) at 0° C. Then the mixture was stirred at 25° C. for 12 h. The mixture was diluted with DCM (50 mL) and quenched by addition of Na₂S₂O₃ solution (50 mL). After stirring for 10 min, The organic layer separated was washed with saturated NaHCO₃ solution (3×50 mL), dried, filtered and concentrated in vacuo to give 6-tert-butyl-3-methyl1-oxido-5,7-dihydropyrrolo[3,4-b]pyridin-1-ium-3,6-dicarboxylate (6.1 g, 19.69 mmol, 91.33% yield, 95% purity) as light yellow solid. ¹H NMR (400 MHz, CDCl₃) δ 1.52 (s, 9H), 3.97 (s, 3H), 4.74-4.88 (m, 4H), 7.68-7.80 (m, 1H), 8.71 (s, 1H). LC-MS: (ES) m/z 295.2 (M+H⁺).

Step g) To a solution of 6-tert-butyl-3-methyl-oxido-5,7-dihydropyrrolo[3,4-b]pyridine-1-ium-3,6-dicarboxylate (6 g, 19.16 mmol) in DMF (180 mL) was added POBr₃ (8.24 g, 28.75 mmol, 2.92 mL) at 0° C. Then the mixture was stirred at 25° C. for 2 h. The mixture was diluted with EtOAc (500 mL) and carefully added to a solution of NaHCO₃ solution (10%, 400 mL). The mixture was extracted with EtOAc (2×100 mL). The combined organic layers were washed with brine (3×100 mL), dried, filtered and concentrated in vacuo to give the residue. The residue was purified by flash silica gel chromatography (ISCO®; 80 g SepaFlash® Silica Flash Column, eluent of 0˜25% ethyl acetate/petroleum ether gradient@80 mL/min) to give 6-tert-butyl-3-methyl 2-bromo-5,7-dihydropyrrolo[3,4-b]pyridine-3,6-dicarboxylate (0.7 g, 1.96 mmol, 10.23% yield, 100% purity) as light yellow gum. ¹H NMR (400 MHz, CDCl₃) δ 1.53 (s, 9H), 3.95-4.00 (m, 3H), 4.62-4.77 (m, 4H), 7.90-8.05 (m, 1H). LC-MS: (ES) m/z 357.1 (M+H⁺).

Step h) To a mixture of 6-tert-butyl-3-methyl-2-bromo-5,7-dihydropyrrolo[3,4-b]pyridine-3,6-dicarboxylate (0.5 g, 1.40 mmol), (4-nitrophenyl)boronic acid (280.39 mg, 1.68 mmol) and Pd(PPh₃)₄ (323.51 mg, 279.96 mol) in dioxane (16 mL) was added a solution of Na₂CO₃ (2 M, 2.10 mL) in at 25° C. Then the mixture was stirred at 70° C. for 12 h. The mixture was diluted with EtOAc (50 mL)/H₂O (50 mL). The mixture was extracted with EtOAc (2×20 mL). The combined organic layers were washed with brine (3×15 mL), dried, filtered and concentrated in vacuo to give the residue. The residue was purified by flash silica gel chromatography (ISCO®; 12 g SepaFlash® Silica Flash Column, eluent of 0˜25% ethyl acetate/petroleum ether gradient@20 mL/min) to give 6-tert-butyl-3-methyl 2-(4-nitrophenyl)-5,7-dihydropyrrolo[3,4-b]pyridine-3,6-dicarboxylate (0.42 g, 1.05 mmol, 70.00% yield) as yellow solid. ¹H NMR (400 MHz, CDCl₃) δ 1.54 (d, J=5.02 Hz, 9H), 3.74 (d, J=4.77 Hz, 3H), 4.72-4.87 (m, 4H), 7.62-7.69 (m, 2H), 8.06-8.17 (m, 1H), 8.31 (d, J=8.78 Hz, 2H). LC-MS: (ES) m/z 400.1 (M+H⁺).

Step i) To a mixture of 6-tert-butyl 3-methyl 2-(4-nitrophenyl)-5,7-dihydropyrrolo[3,4-b] pyridine-3,6-dicarboxylate (0.23 g, 575.87 mol) in MeOH (5 mL)/THF (2 mL) was added a solution of LiOH H₂O (120.83 mg, 2.88 mmol, 413.12 μL) in H₂O (0.5 mL) at 25° C. Then the mixture was stirred at 25° C. for 3 h. The mixture was concentrated in vacuo to give the residue. The residue was diluted with MTBE (30 mL) and acidified to pH=4-5 by addition of citric acid aqueous solution. The mixture was extracted with EtOAc (2×10 mL). The combined organic layers were dried over anhydrous Na₂SO₄, filtered and concentrated in vacuo to give the target compound 6-tert-butoxycarbonyl-2-(4-nitrophenyl)-5,7-dihydropyrrolo[3,4-b]pyridine-3-carboxylic acid (220 mg, 570.88 mol, 99.13% yield) as off-white solid. ¹H NMR (400 MHz, DMSO-d₆) δ 1.48 (d, J=4.27 Hz, 9H), 4.66 (br d, J=11.29 Hz, 2H), 4.72 (br d, J=12.30 Hz, 2H), 7.77 (dd, J=8.78, 2.01 Hz, 2H), 8.23 (d, J=9.03 Hz, 1H), 8.30 (d, J=8.53 Hz, 2H). LC-MS: (ES) m/z 386.1 (M+H⁺).

Step j) To a mixture of 6-tert-butoxycarbonyl-2-(4-nitrophenyl)-5,7-dihydropyrrolo[3,4-b] pyridine-3-carboxylic acid (200 mg, 518.98 mol, 31.27 μL), HATU (236.80 mg, 622.78 mol) in DCM (5 mL) was added successively with DIEA (134.15 mg, 1.04 mmol, 180.79 μL), 4-methyl-3-(trifluoromethyl)aniline (109.08 mg, 622.78 mol, 89.41 L). Then the mixture was stirred at 25° C. for 12 h. The reaction mixture was diluted with DCM (30 mL) and washed with H₂O (2×10 mL). The organic layer was dried, filtered and concentrated in vacuo to give the residue. The residue was purified by flash silica gel chromatography (ISCO®; 4 g SepaFlash® Silica Flash Column, eluent of 0˜35% ethyl acetate/petroleum ether gradient@20 mL/min) to give tert-butyl 3-[[4-methyl-3-(trifluoromethyl)phenyl]carbamoyl]-2-(4-nitrophenyl)-5,7-dihydropyrrolo[3,4-b]pyridine-6-carboxylate (0.3 g, 497.69 mol, 95.90% yield, 90% purity) as light yellow solid. ¹H NMR (400 MHz, CDCl₃) δ 1.54 (d, J=5.77 Hz, 9H), 2.44 (s, 3H), 2.79 (s, 7H), 4.77 (br d, J=15.06 Hz, 4H), 7.24 (br d, J=8.28 Hz, 1H), 7.47 (br dd, J=19.95, 7.65 Hz, 1H), 7.56 (br s, 1H), 7.87-7.98 (m, 3H), 8.29 (d, J=8.78 Hz, 2H). LC-MS: (ES) m/z 543.2 (M+H⁺).

Step k) To a mixture of tert-butyl3-[[4-methyl-3-(trifluoromethyl)phenyl]carbamoyl]-2-(4-nitro phenyl)-5,7-dihydropyrrolo[3,4-b]pyridine-6-carboxylate (0.3 g, 497.69 mol) in Dioxane (3 mL) was added HCl/dioxane (4 M, 1.87 mL). Then the mixture was stirred at 25° C. for 2 h. The reaction mixture was concentrated in vacuo to give the desired compound N-[4-methyl-3-(trifluoromethyl)phenyl]-2-(4-nitrophenyl)-6,7-dihydro-5H-pyrrolo[3,4-b]pyridine-3-carboxamide (0.22 g, 450.24 mol, 90.47% yield, 98% purity, HCl) as light brown solid. ¹H NMR (400 MHz, DMSO-d₆) δ 2.38 (d, J=1.00 Hz, 3H), 4.64 (br s, 2H), 4.70 (br s, 2H), 7.39 (d, J=8.53 Hz, 1H), 7.62-7.68 (m, 1H), 7.86-7.90 (m, 2H), 7.91 (d, J=2.01 Hz, 1H), 8.20 (s, 1H), 8.30 (d, J=9.03 Hz, 2 H), 10.23 (br s, 2H), 10.91 (s, 1H). LC-MS: (ES) m/z 443.1 (M+H⁺).

Step l) To a solution of N-[4-methyl-3-(trifluoromethyl)phenyl]-2-(4-nitrophenyl)-6,7-dihydro-5H-pyrrolo[3,4-b]pyridine-3-carboxamide (110 mg, 229.72 mol, HCl) and HCHO (in H₂O) (55.93 mg, 689.15 mol, 51.31 μL) in DCE (5 mL) was added successively TEA (46.49 mg, 459.43 mol, 63.95 μL) and NaBH(OAc)₃ (146.06 mg, 689.15 mol). Then the mixture was stirred at 25° C. for 12 h. The mixture was diluted with DCM (30 mL) and alkalified to pH=8-9 and extracted with DCM (3×20 mL). The combined organic layers were dried, filtered and concentrated in vacuo to give the desired compound 6-methyl-N-[4-methyl-3-(trifluoromethyl)phenyl]-2-(4-nitrophenyl)-5,7-dihydropyrrolo[3,4-b]pyridine-3-carboxamide (110 mg, 216.91 mol, 94.42% yield, 90% purity) as light brown solid. ¹H NMR (400 MHz, DMSO-d₆) δ 2.38 (d, J=1.25 Hz, 3H), 2.56 (s, 3H), 3.96 (s, 2H), 3.99 (s, 2H), 7.38 (d, J=8.28 Hz, 1H), 7.65 (br d, J=8.03 Hz, 1H), 7.84-7.89 (m, 2H), 7.91 (d, J=2.01 Hz, 1H), 8.00 (s, 1H), 8.25-8.30 (m, 2H), 10.73 (s, 1H). LC-MS: (ES) m z 457.1 (M+H⁺).

Step m) To a solution of 6-methyl-N-[4-methyl-3-(trifluoromethyl)phenyl]-2-(4-nitrophenyl)-5,7-dihydropyrrolo[3,4-b]pyridine-3-carboxamide (110 mg, 216.91 mol) in MeOH (8 mL) was added successively with HCl/dioxane (4 M, 108.45 μL) and PtO₂ (24.63 mg, 108.45 mol) under N₂. The suspension was degassed under vacuum and purged with H₂ several times. The mixture was stirred under H₂ (15 psi) at 25° C. for 1.5 h. The mixture was diluted with MeOH (30 mL) and filtered through a pad of Celite and the filtrate was concentrated in vacuo to give the residue. The residue was diluted with DCM (50 mL) and alkalified to pH=9˜10 by saturated NaHCO₃ solution. The organic layers separated was dried, filtered and concentrated in vacuo to give the desired compound cis-2-(4-aminophenyl)-6-methyl-N-[4-methyl-3-(trifluoro methyl)phenyl]-1,2,3,4,4a,5,7,7a-octahydropyrrolo[3,4-b]pyridine-3-carboxamide (100 mg, crude) as light brown gum. ¹H NMR (400 MHz, DMSO-d₆) δ 2.35 (br s, 4H), 2.90 (br s, 3H), 3.91-4.19 (m, 2H), 4.60 (br s, 1H), 6.57 (br d, J=6.78 Hz, 2H), 7.18 (br d, J=7.78 Hz, 2H), 7.32 (br d, J=7.28 Hz, 1H), 7.40-7.55 (m, 1H), 7.81-7.93 (m, 1H). LC-MS: (ES) m/z 433.1 (M+H⁺).

Step n) To a mixture of cis-2-(4-aminophenyl)-6-methyl-N-[4-methyl-3-(trifluoro-methyl)phenyl]-1,2,3,4,4a,5,7,7a-octahydropyrrolo[3,4-b]pyridine-3-carboxamide (90 mg, 166.48 μmol) in MeOH (4.5 mL) was added cyclopentanone (16.80 mg, 199.78 mol, 17.69 μL), HOAc (15.00 mg, 249.72 mol, 14.28 μL) and NaBH₃CN (31.39 mg, 499.44 mol) in one portion at 0° C. under N₂. The mixture was stirred at 25° C. for 16 h. The mixture was diluted with DCM (30 mL) and alkalified to pH=8˜9 by saturated NaHCO₃ solution and extracted with DCM (3×30 mL). The combined organic layers were washed with brine, dried, filtered and concentrated in vacuo to give the crude product. The crude product was purified by silica gel column chromatography (eluted with DCM/MeOH/NH₃·H₂O from 100/I/0.1 to 20/I/0.02) to give cis-2-[4-(cyclopentylamino)phenyl]-6-methyl-N-[4-methyl-3-(trifluoromethyl)phenyl]-1,2,3,4,4a,5,7,7a-octahydropyrrolo[3,4-b]pyridine-3-carboxamide (60 mg, 113.86 mol, 68.39% yield, 95% purity) as light yellow solid. ¹H NMR (400 MHz, DMSO-d₆) δ 1.28-1.39 (m, 2H), 1.40-1.55 (m, 3H), 1.56-1.66 (m, 2H), 1.77-1.89 (m, 2H), 1.92-1.99 (m, 1H), 2.00-2.08 (m, 1H), 2.26 (s, 3H), 2.34-2.37 (m, 3H), 2.57-2.69 (m, 3H), 2.73-2.80 (m, 1H), 3.52-3.65 (m, 1H), 3.80 (br d, J=4.52 Hz, 1H), 5.35 (d, J=6.53 Hz, 1H), 6.40 (d, J=8.53 Hz, 2H), 6.99-7.04 (m, 2H), 7.28 (d, J=8.53 Hz, 1H), 7.58 (br d, J=8.03 Hz, 1H), 7.76 (s, 1H), 11.29 (br s, 1H). LC-MS: (ES) m/z 501.2 (M+H⁺).

Step o) To a solution of cis-2-[4-(cyclopentylamino)phenyl]-6-methyl-N-[4-methyl-3-(trifluoromethyl)phenyl]-,2,3,4,4a,5,7,7a-octahydropyrrolo[3,4-b]pyridine-3-carboxamide (60 mg, 119.86 mol) and DIEA (30.98 mg, 239.71 mol, 41.75 μL) in DCM (5 mL) was added dropwise of a solution of 2-fluoro-6-methyl-benzoyl chloride (20.69 mg, 119.86 mol) in DCM (3 mL) at 0° C. The mixture was stirred at 0° C. for 20 min. The mixture was diluted with DCM (30 mL), washed with H₂O (2×10 mL), dried, filtered and concentrated in vacuo to give the crude product. The crude product was purified by prep-HPLC (column: Phenomenex Gemini-NX 150×30 mm×5 μm; mobile phase: [water (0.05% HCl)-ACN]; B %: 21%-41%, 9 min) to give cis-2-[4-(cyclopentyl-amino)phenyl]-1-(2-fluoro-6-methyl-benzoyl)-6-methyl-N-[4-methyl-3-(trifluoromethyl)phenyl]-3,4,4a,5,7,7a-hexahydro-2H-pyrrolo[3,4-b]pyridine-3-carboxamide (20 mg, 29.12 mol, 24.29% yield, 98% purity, HCl) as light yellow solid. ¹H NMR (400 MHz, METHANOL-d₄) δ 1.44 (br s, 1 H), 1.67 (br s, 5H), 1.76-2.06 (m, 5H), 2.22-2.55 (m, 6H), 2.62-2.99 (m, 3H), 3.02-3.25 (m, 3H), 3.62 (br s, 1H), 3.77-4.23 (m, 3H), 5.24 (br s, 1H), 6.32-6.81 (m, 1H), 6.98 (br s, 1H), 7.05-7.34 (m, 5H), 7.35-7.81 (m, 3H), 7.87 (br s, 1H), 9.93-10.44 (m, 1 H). LC-MS: (ES) m/z 637.23 (M+H⁺).

### Example S160: Synthesis of cis-2-(4-(cyclopentyl(methyl)amino)phenyl)-1-(2-fluoro-6-methylbenzoyl)-6-methyl-N-(4-methyl-3-(trifluoromethyl)phenyl)octahydro-1H-pyrrolo[3,4-b]pyridine-3-carboxamide (Compound No. 144)

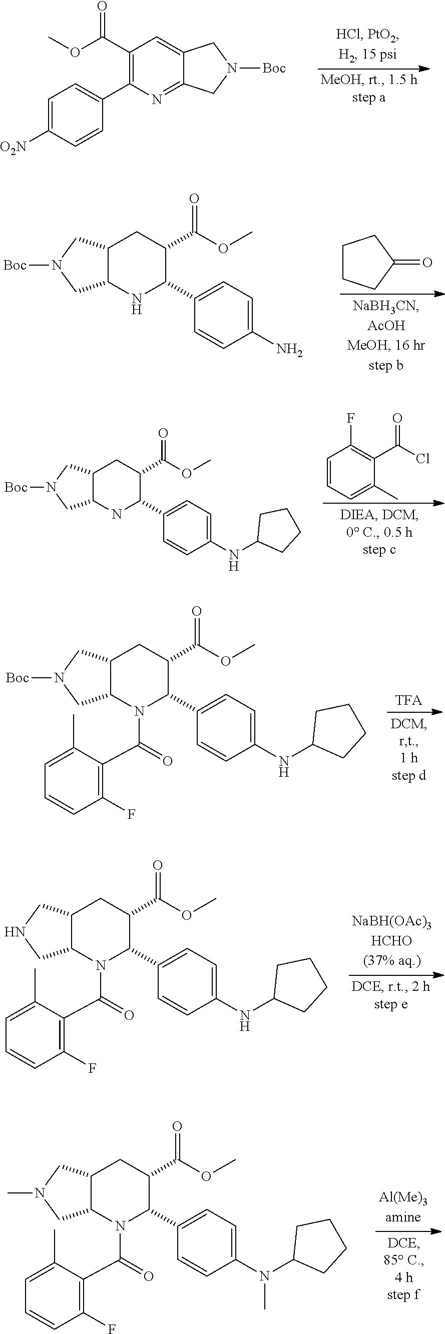

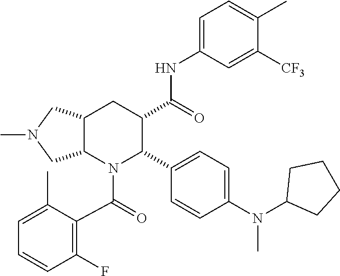

Step a) To a solution of 6-tert-butyl-3-methyl-2-(4-nitrophenyl)-5H-pyrrolo[3,4-b]pyridine-3,6(7H)-dicarboxylate (500 mg, 1.25 mmol) in MeOH (25 mL) was added successively with HCl/dioxane (4 M, 625.94 μL) and PtO₂ (142.14 mg, 625.94 mol) under N₂. The suspension was degassed under vacuum and purged with H₂ several times. The mixture was stirred under H₂ (15 psi) at 25° C. for 1.5 hours. The mixture was diluted with MeOH and filtered through a pad of Celite and the filtrate was concentrated in vacuo to give the residue. The residue was diluted with DCM (50 mL) and alkalified to pH=9˜10 by addition of saturated NaHCO₃ aqueous solution. The organic layer separated was dried, filtered and concentrated in vacuo to give the crude product. The crude product was purified by flash silica gel chromatography (ISCO®; 4 g SepaFlash® Silica Flash Column, eluent of 0˜10% MeOH/DCM gradient@20 mL/min) to give cis-6-tert-butyl 3-methyl 2-(4-aminophenyl)-1,2,3,4,4a,5,7,7a-octahydropyrrolo [3,4-b]pyridine-3,6-dicarboxylate (0.2 g, 511.37 mol, 40.85% yield, 96% purity) as light brown gum. ¹H NMR (400 MHz, CDCl₃) δ 1.47 (s, 9H), 2.23 (br dd, J=13.45, 3.67 Hz, 3H), 2.89 (br d, J=4.16 Hz, 1H), 3.37-3.51 (m, 6H), 3.54-3.66 (m, 2H), 3.90 (br d, J=4.40 Hz, 1H), 6.64 (d, J=8.31 Hz, 2H), 7.03-7.15 (m, 2H). LC-MS: (ES) m/z 376.3 (M+H⁺).

Step b) To a mixture of cis-6-tert-butyl 3-methyl-2-(4-aminophenyl)-1,2,3,4,4a,5,7,7a-octahydropyrrolo[3,4-b]pyridine-3,6-dicarboxylate (200 mg, 532.68 mol) in MeOH (8 mL) was added cyclopentanone (53.77 mg, 639.21 mol, 56.60 μL), HOAc (47.98 mg, 799.02 mol, 45.70 μL) and NaBH₃CN (100.42 mg, 1.60 mmol) n one portion at 0° C. The mixture was stirred at 25° C. for 16 h. The mixture was diluted with DCM (30 mL) and alkalified to pH=8-9 and extracted with DCM (3×30 mL). The combined organic layers were washed with brine, dried, filtered and concentrated in vacuo to give the desired compound cis-6-tert-butyl-3-methyl 2-[4-(cyclopentyl-amino)phenyl]-1,2,3,4,4a,5,7,7a-octahydropyrrolo[3,4-b]pyridine-3,6-dicarboxylate (0.23 g, 492.59 mol, 92.47% yield, 95% purity) as light yellow gum. ¹H NMR (400 MHz, CDCl₃) δ 1.48 (s, 9H), 1.71 (br d, J=7.09 Hz, 2H), 1.94-2.02 (m, 4H), 2.12-2.28 (m, 5H), 2.89 (br d, J=4.40 Hz, 1H), 3.36-3.53 (m, 7H), 3.55-3.65 (m, 1H), 3.72-3.82 (m, 1H), 3.89 (br d, J=4.89 Hz, 1H), 6.55 (d, J=8.31 Hz, 2H), 7.08-7.16 (m, 2H). LC-MS: (ES) m/z 444.3 (M+H⁺).

Step c) To a solution of cis-6-tert-butyl-3-methyl 2-[4-(cyclopentylamino)phenyl]-1,2,3,4,4a,5,7,7a-octahydropyrrolo[3,4-b]pyridine-3,6-dicarboxylate (0.23 g, 492.59 μmol) and DIEA (127.32 mg, 985.17 mol, 171.60 μL) in DCM (10 mL) was added dropwise of a solution of 2-fluoro-6-methyl-benzoyl chloride (80.76 mg, 467.96 mol) in DCM (5 mL) at 0° C. The mixture was stirred at 0° C. for 20 min. The mixture was diluted with DCM (30 mL), washed with H₂O (2×10 mL), dried, filtered and concentrated in vacuo to give the crude product. The crude product was purified by prep-HPLC (column: YMC Triart C18 150×25 mm×5 μm; mobile phase: [water (10 mM NH₄HCO₃)-ACN]; B %: 63%-93%, 9.5 min) to give cis-6-tert-butyl-3-methyl-2-[4-(cyclopentylamino)phenyl]-1-(2-fluoro-6-methyl-benzoyl)-3,4,4a,5,7,7a-hexahydro-2H-pyrrolo[3,4-b]pyridine-3,6-dicarboxylate (0.16 g, 220.80 mol, 32.00% yield, 80% purity) as off-white solid. ¹H NMR (400 MHz, DMSO-d₆) δ 1.13-1.37 (m, 9H), 1.38-1.45 (m, 2H), 1.49-1.69 (m, 3H), 1.80-1.93 (m, 2H), 2.16-2.36 (m, 4H), 2.95-3.26 (m, 2H), 3.58-3.70 (m, 3H), 3.88-4.01 (m, 1H), 5.63 (br s, 1H), 6.40-6.53 (m, 2H), 6.94 (d, J=8.56 Hz, 1H), 7.09-7.22 (m, 2H), 7.30-7.43 (m, 1H). LC-MS: (ES) m/z 480.2 (M+H⁺).

Step d) To a solution of cis-6-tert-butyl-3-methyl2-[4-(cyclopentylamino)phenyl]-1-(2-fluoro-6-methyl-benzoyl)-3,4,4a,5,7,7a-hexahydro-2H-pyrrolo[3,4-b]pyridine-3,6-dicarboxylate (160.00 mg, 220.80 mol) in DCM (4 mL) was added TFA (616.08 mg, 5.40 mmol, 400.05 μL). Then the mixture was stirred at 25° C. for 1 h. The mixture was concentrated in vacuo to give the residue. The residue was dissolved in DCM (30 mL) and alkalified to pH=8-9. The organic layer separated was dried over anhydrous Na₂SO₄, filtered and concentrated in vacuo to give cis-methyl 2-[4-(cyclopentylamino)phenyl]-1-(2-fluoro-6-methyl-benzoy)-2,3,4,4a,5,6,7,7a-octahydropyrrolo-[3,4-b]pyridine-3-carboxylate (120 mg, 200.17 mol, 90.66% yield, 80% purity) as light yellow gum. LC-MS: (ES) m/z 480.2 (M+H⁺).

Step e) To a solution of cis-methyl-2-[4-(cyclopentylamino)phenyl]-1-(2-fluoro-6-methyl-benzoyl)-2,3,4,4a,5,6,7,7a-octahydropyrrolo[3,4-b]pyridine-3-carboxylate (110 mg, 229.37 mol) and HCHO (in H₂O) (11.17 mg, 137.62 mol, 10.25 μL) in DCE (4 mL) was added NaBH(OAc)₃ (145.84 mg, 688.10 mol). Then the mixture was stirred at 25° C. for 1 h. The mixture was diluted with DCM (30 mL) and alkalified to pH=8-9 and extracted with DCM (3×10 mL). The combined organic layers were dried, filtered and concentrated in vacuo to give the crude product. The crude product was purified by prep-HPLC (column: Phenomenex Gemini-NX 150×30 mm×5 μm; mobile phase: [water (0.1% TFA)-ACN]; B %: 6%-36%, 10 min). The eluent was alkalified to pH=8-9 and extracted with DCM (3×20 mL). The combined organic layers were dried, filtered and concentrated in vacuo to give cis-methyl 2-[4-[cyclopentyl(methyl)amino]phenyl]-1-(2-fluoro-6-methyl-benzoyl)-6-methyl-3,4,4a,5,7,7a-hexahydro-2H-pyrrolo[3,4-b]pyridine-3-carboxylate (60 mg, 117.01 mol, 51.02% yield, 99% purity) as white solid. LC-MS: (ES) m/z 508.3 (M+H⁺).

Step f) To a solution of 4-methyl-3-(trifluoromethyl)aniline (51.75 mg, 295.49 mol, 42.42 μL) in DCE (1 mL) was added Al(CH₃)₃ (in toluene) (2 M, 177.29 μL) at 0° C. After stirring for 30 min at 25° C., a solution of cis-methyl 2-[4-[cyclopentyl(methyl)amino]phenyl]-1-(2-fluoro-6-methyl-benzoyl)-6-methyl-3,4,4a,5,7,7a-hexahydro-2H-pyrrolo[3,4-b]pyridine-3-carboxylate (60 mg, 118.19 mol) in DCE (1 mL) was added. The mixture was stirred at 85° C. for 3 h 30 min. The mixture was diluted with DCM (30 mL), and quenched by addition of saturated NaHCO₃ solution (10 mL). After stirring for 15 min at RT, the mixture was extracted with DCM (3×10 mL). The combined organic layers were dried, filtered and concentrated in vacuo to give the crude product. The crude was purified by prep-HPLC (column: Phenomenex Gemini-NX 150×30 mm×5 μm; mobile phase: [water (0.05% HCl)-ACN]; B %: 13%-53%, 10 min) to give target compound cis-2-[4-[cyclopentyl(methyl)amino]phenyl]-1-(2-fluoro-6-methyl-benzoyl)-6-methyl-N-[4-methyl-3-(trifluoromethyl)phenyl]-3,4,4a,5,7,7a-hexahydro-2H-pyrrolo[3,4-b]pyridine-3-carboxamide (42 mg, 59.89 mol, 50.67% yield, 98% purity, HCl) as white solid. ¹H NMR (400 MHz, METHANOL-d₄) δ 1 1.44-1.53 (m, 1H), 1.61-2.06 (m, 7H), 2.08-2.32 (m, 2 H), 2.33-2.47 (m, 5H), 2.49 (s, 1H), 2.73-2.80 (m, 1H), 2.82-2.93 (m, 1H), 3.01 (br d, J=8.28 Hz, 1H), 3.12 (s, 1H), 3.18-3.23 (m, 2H), 3.25 (br d, J=2.76 Hz, 1H), 3.31 (s, 2 H), 3.50-3.74 (m, 1H), 3.84-4.01 (m, 1H), 4.03-4.24 (m, 2H), 5.28 (br d, J=7.03 Hz, 1 H), 6.43-6.63 (m, 0.5 H), 6.68-6.78 (m, 0.5 H), 7.06 (br t, J=8.41 Hz, 1H), 7.12-7.19 (m, 1H), 7.22-7.36 (m, 3H), 7.37-7.54 (m, 2H), 7.54-7.64 (m, 1H), 7.65-7.73 (m, 1H), 7.84-8.01 (m, 1H). LC-MS: (ES) m/z 651.4 (M+H⁺).

### Example S161: Synthesis of (2R,3S,4aS,7aS)-2-(4-(cyclopentylamino)phenyl)-1-(2-fluoro-6-methylbenzoyl)-N-(4-methyl-3-(trifluoromethyl)phenyl)-6-(2,2,2-trifluoroethyl)octahydro-1H-pyrrolo[3,4-b]pyridine-3-carboxamide (Compound No. 145)

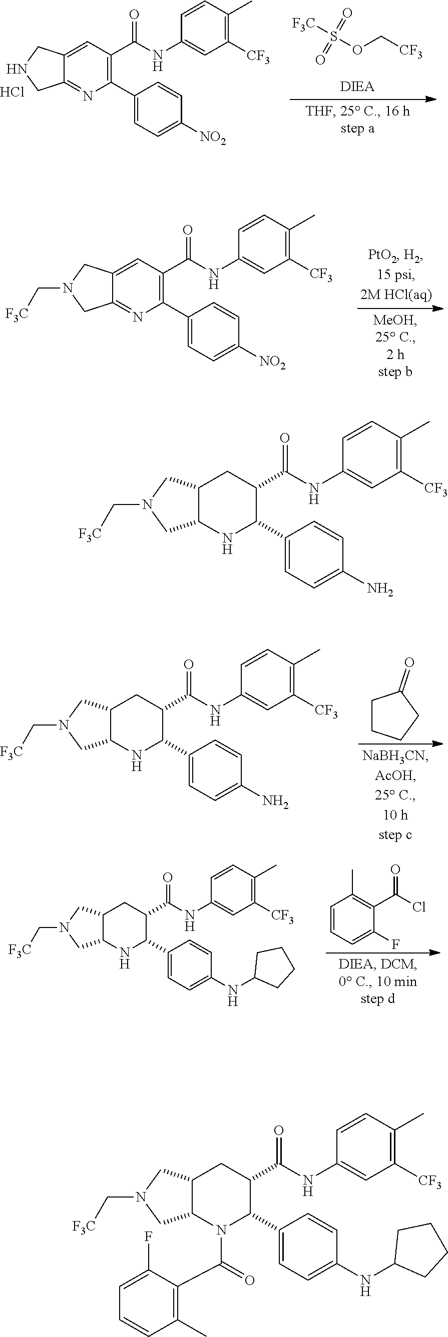

Step a) To a mixture of N-[4-methyl-3-(trifluoromethyl)phenyl]-2-(4-nitrophenyl)-6,7-dihydro-5H-pyrrolo[3,4-b]pyridine-3-carboxamide (120 mg, 232.87 mol, 2HCl) in THF (5 mL) was added 2,2,2-trifluoroethyl trifluoromethanesulfonate (108.10 mg, 465.74 mol) and DIEA (60.19 mg, 465.74 mol, 81.12 μL) at 25° C. under N₂. The mixture was stirred at 25° C. for 16 h. Then concentrated to get a residue. The residue was purified by prep-TLC (SiO₂, petroleum ether/ethyl acetate=1/1). N-[4-methyl-3-(trifluoromethyl)phenyl]-2-(4-nitrophenyl)-6-(2,2,2-trifluoroethyl)-5,7-dihydropyrrolo[3,4-b]pyridine-3-carboxamide (120 mg, 228.83 mol, 98.26% yield) was obtained as a white solid. LC-MS: (ES) m/z 525.1 (M+H⁺). ¹H NMR (400 MHz, CDCl₃) δ ppm 1.48-1.74 (m, 22H), 2.45 (br s, 3H), 3.35-3.62 (m, 2H), 4.36 (br s, 4H), 7.13 (br s, 1H), 7.39 (br s, 1H), 7.52 (br s, 1H), 7.92 (br s, 3H), 8.24-8.40 (m, 2H).

Step b) To a solution of N-[4-methyl-3-(trifluoromethyl)phenyl]-2-(4-nitrophenyl)-6-(2,2,2-trifluoroethyl)-5,7-dihydropyrrolo[3,4-b]pyridine-3-carboxamide (120 mg, 228.83 mol), PtO₂ (25.98 mg, 114.41 mol) in MeOH (5 mL) was added HCl(aq) (2 M, 228.83 μL) under N₂. The suspension was degassed under vacuum and purged with H₂ several times. The mixture was stirred under H₂ (15 psi) at 20° C. for 2 h. The mixture was filtered and concentrated under reduced pressure to give the residue. The residue was purified by prep-TLC (SiO₂, DCM:MeOH=10:1). 2-(4-aminophenyl)-N-[4-methyl-3-(trifluoromethyl)phenyl]-6-(2,2,2-t rifluoroethyl)-1,2,3,4,4a,5,7,7a-octahydropyrrolo[3,4-b]pyridine-3-carboxamide (45 mg, 84.52 mol, 36.94% yield, 94% purity) was obtained as a light yellow oil. LC-MS: (ES) m/z 501.2 (M+H⁺).

Step c) To a mixture of 2-(4-aminophenyl)-N-[4-methyl-3-(trifluoromethyl)phenyl]-6-(2, 2,2-trifluoroethyl)-1,2,3,4,4a,5,7,7a-octahydropyrrolo[3,4-b]pyridine-3-carboxamide (45 mg, 89.91 mol) and cyclopentanone (9.08 mg, 107.90 mol, 9.55 μL) in MeOH (2 mL) was added NaBH₃CN (16.95 mg, 269.74 mol) at 20° C. under N₂. Then AcOH (10.80 mg, 179.83 mol, 10.28 μL) was added to the mixture, the mixture was stirred at 20° C. for 10 h. The reaction mixture was partitioned between saturated NaHCO₃(aq) (20 ml) and DCM (20 mL). The organic phase was separated, dried filtered and concentrated under reduced pressure to give a residue. The residue was purified by prep-TLC (SiO₂, DCM:MeOH=10:1). 2-[4-(cyclopentylamino)phenyl]-N-[4-methyl-3-(trifluoromethyl)phenyl]-6-(2,2,2-trifluoroethyl)-1,2,3,4,4a,5,7,7a-octahydropyrrolo[3,4-b]pyridine-3-carboxamide (25 mg, 43.97 umol, 48.90% yield, 100% purity) was obtained as a light yellow oil. LC-MS: (ES) m z 567.3 (M+H⁺).

Step d) To a mixture of 2-[4-(cyclopentylamino)phenyl]-N-[4-methyl-3-(trifluoro-methyl)phenyl]-6-(2,2,2-trifluoroethyl)-1,2,3,4,4a,5,7,7a-octahydropyrrolo[3,4-b]pyridine-3-carboxamide (25 mg, 43.97 mol), DIEA (11.36 mg, 87.94 mol, 15.32 μL) in DCM (1 mL) was added 2-fluoro-6-methyl-benzoyl chloride (7.59 mg, 43.97 mol) at 0° C. under N₂. The mixture was stirred at 0° C. for 10 min, then concentrated to get a residue. The residue was purified by prep-HPLC (column: Phenomenex Gemini-NX 150*30 mm*5 μm; mobile phase: [water (0.05% HCl)-ACN]; B %: 36%-66%, 7 min). 2-[4-(cyclopentylamino)phenyl]-1-(2-fluoro-6-methyl-benzoyl)-N-[4-methyl-3-(trifluoromethyl)phenyl]-6-(2,2,2-trifluoroethyl)-3,4,4a,5,7,7a-hexahydro-2H-pyrrolo[3,4-b]pyridine-3-carboxamide (3 mg, 4.05 mol, 9.21% yield, 100% purity, HCl) was obtained as a light yellow solid. LC-MS: (ES) m/z 705.3 (M+H⁺). ¹H NMR (400 MHz, METHANOL-d₄) δ ppm 1.28-1.48 (m, 5H), 1.52 (br d, J=3.76 Hz, 1H), 1.87-2.08 (m, 2H), 2.21-2.34 (m, 4H), 2.37-2.56 (m, 1H), 2.37-2.56 (m, 4H), 2.84-2.98 (m, 2H), 3.09 (br d, J=2.76 Hz, 2 H), 3.19 (br d, J=10.29 Hz, 1H), 3.26-3.31 (m, 1H), 3.43-3.54 (m, 1H), 3.90-4.05 (m, 1 H), 4.64 (d, J=3.51 Hz, 1H), 6.31-6.44 (m, 1H), 6.47-6.58 (m, 1H), 6.65-6.79 (m, 1H), 7.25-7.38 (m, 3H), 7.39-7.57 (m, 3H), 7.83-7.94 (m, 1H).

### Example S162: Synthesis of (2R,3S,4aS,7aS)-6-acetyl-2-(4-(cyclopentylamino)phenyl)-1-(2-fluoro-6-methylb enzoyl)-N-(4-methyl-3-(trifluoromethyl)phenyl)octahydro-1H-pyrrolo[3,4-b]pyridine-3-carboxamide (Compound No. 146)

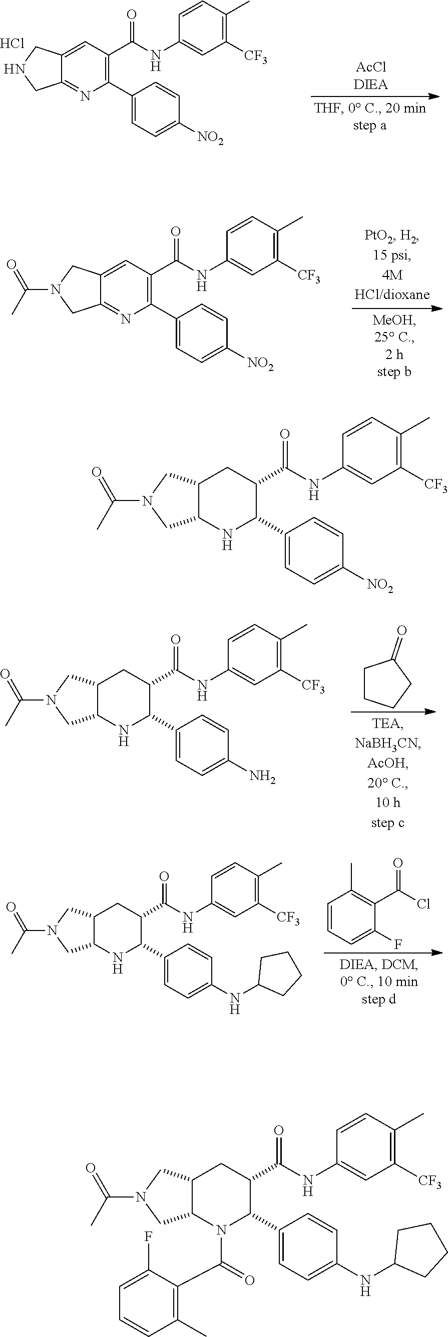

Step a) To a mixture of N-[4-methyl-3-(trifluoromethyl)phenyl]-2-(4-nitrophenyl)-6,7-dihydro-5H-pyrrolo[3,4-b]pyridine-3-carboxamide (100 mg, 194.06 mol, 2HCl), TEA (58.91 mg, 582.17 mol, 81.03 μL) in DCM (1 mL) was added acetyl chloride (30.47 mg, 291.09 mol, 27.70 μL) at 0° C. under N₂. The mixture was stirred at 0° C. for 20 min. The reaction mixture was concentrated under reduced pressure to give the residue. The residue was purified by prep-TLC (SiO₂, DCM:MeOH=10:1). 6-acetyl-N-[4-methyl-3-(trifluorom ethyl)p henyl]-2-(4-nitrophenyl)-5,7-dihydropyrrolo[3,4-b]pyridine-3-carboxamide (85 mg, 175.47 mol, 90.42% yield, 100% purity) was obtained as a light yellow gum. LC-MS: (ES) m/z 485.1 (M+H⁺).

Step b) To a solution of 6-acetyl-N-[4-methyl-3-(trifluoromethyl)phenyl]-2-(4-nitrophenyl)-5,7-dihydropyrrolo[3,4-b]pyridine-3-carboxamide (85 mg, 175.47 mol), PtO₂ (19.92 mg, 87.73 mol) in MeOH (5 mL) was added HCl/dioxane (4 M, 87.73 μL) under N₂. The suspension was degassed under vacuum and purged with H₂ several times. The mixture was stirred under H₂ (15 psi) at 20° C. for 2 h. showed the desired product was detected. The mixture was filtered and concentrated under reduced pressure to give the desired product. 6-acetyl-2-(4-aminophenyl)-N-[4-methyl-3-(trifluoromethyl)phenyl]-1,2,3,4,4a,5,7,7a-octahydropyrrolo[3,4-b]pyridine-3-carboxamide (80 mg, crude) was obtained as a light yellow oil. LC-MS: (ES) m/z 461.2 (M+H⁺).

Step c) To a mixture of 6-acetyl-2-(4-aminophenyl)-N-[4-methyl-3-(trifluoromethyl)-phenyl]-1,2,3,4,4a,5,7,7a-octahydropyrrolo [3,4-b]pyridine-3-carboxamide (80 mg, 160.98 μmol, HCl) and cyclopentanone (14.90 mg, 177.08 mol, 15.68 μL) in MeOH (2 mL) was added TEA (32.58 mg, 321.96 mol, 44.81 μL) and AcOH (19.33 mg, 321.96 mol, 18.41 μL) at 20° C. under N₂. Then NaBH₃CN (30.35 mg, 482.94 mol) was added to the mixture and the mixture was stirred at 20° C. for 10 h. The reaction mixture was partitioned between saturated NaHCO₃(aq) (20 ml) and DCM (20 mL). The organic phase was separated, dried filtered and concentrated under reduced pressure to give a residue. The residue was purified by prep-TLC (SiO₂, DCM:MeOH=10:1, plate 1). 6-acetyl-2-[4-(cyclopentylamino)phenyl]-N-[4-methyl-3-(trifluoromethyl)phenyl]-1,2,3,4,4a,5,7,7a-octahydropyrrolo[3,4-b]pyridine-3-carboxamide (20 mg, crude) was obtained as a light yellow oil. LC-MS: (ES) m/z 529.3 (M+H⁺).

Step d) To a mixture of 6-acetyl-2-[4-(cyclopentylamino)phenyl]-N-[4-methyl-3-(trifluoromethyl)phenyl]-1,2,3,4,4a,5,7,7a-octahydropyrrolo[3,4-b]pyridine-3-carboxamide (20 mg, 37.84 mol), DIEA (9.78 mg, 75.67 μmol, 13.18 μL) in DCM (1 mL) was added 2-fluoro-6-methyl-benzoyl chloride (6.53 mg, 37.84 mol) at 0° C. under N₂. The mixture was stirred at 0° C. for 10 min, then concentrated to get a residue. The residue was purified by prep-HPLC (column: Phenomenex Gemini-NX 150*30 mm*5 μm; mobile phase: [water (0.05% H C1)-ACN]; B %: 25%-55%, 7 min). 6-acetyl-2-[4-(cyclopentylamino)phenyl]-1-(2-fluoro-6-methyl-benzoyl)-N-[4-methyl-3-(trifluoromethyl)phenyl]-3,4,4a,5,7,7a-hexahydro-2H-pyrrolo[3,4-b]pyridine-3-carboxamide (3 mg, 4.15 mol, 10.97% yield, 97% purity, HCl) was obtained as a light yellow solid. LC-MS: (ES) m/z 665.3 (M+H⁺). ¹H NMR (400 MHz, METHANOL-d₄) δ ppm 1.26-1.61 (m, 6H), 1.90-2.05 (m, 2H), 2.07-2.22 (m, 3H), 2.26 (d, J=16.06 Hz, 3H), 2.30-2.39 (m, 1H), 2.39-2.50 (m, 4H), 2.51-2.65 (m, 1H), 2.91-3.20 (m, 2H), 3.66-3.83 (m, 2H), 3.89-4.04 (m, 2H), 4.05-4.21 (m, 1H), 4.69 (br s, 1 H), 6.26-6.60 (m, 1H), 6.72 (br s, 1H), 7.26-7.39 (m, 3H), 7.40-7.56 (m, 3H), 7.58-8.09 (m, 2H).

### Example S163: Synthesis of (2R,3S,4aS,7aS)-2-(4-(cyclopentylamino)phenyl)-1-(2-fluoro-6-methylbenzoyl)-N-(4-methyl-3-(trifluoromethyl)phenyl)octahydro-1H-pyrrolo[3,4-b]pyridine-3-carboxamide (Compound No. 39)

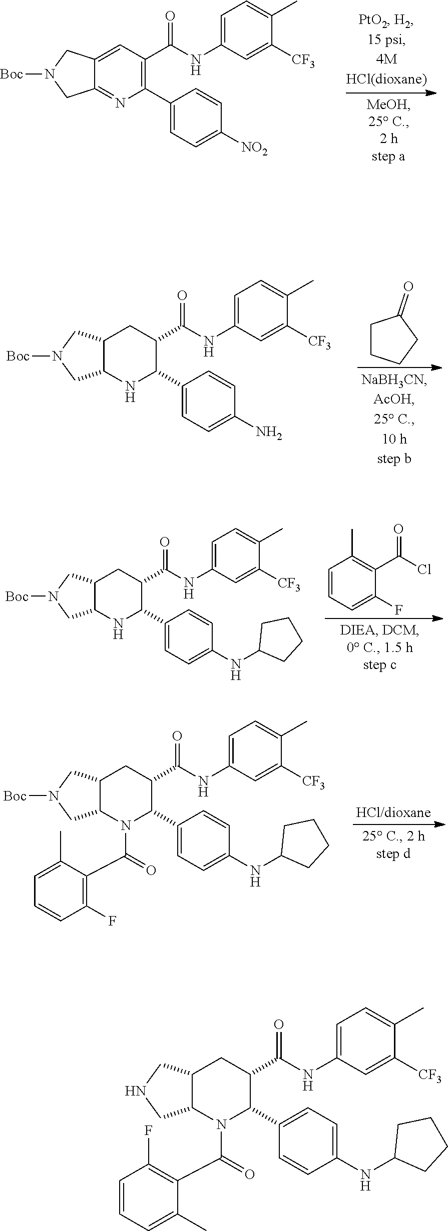

Step a) To a solution of tert-butyl 3-[[4-methyl-3-(trifluoromethyl)phenyl]carbamoyl]-2-(4-nitrophenyl)-5,7-dihydropyrrolo[3,4-b]pyridine-6-carboxylate (50 mg, 92.17 mol), PtO₂ (10.46 mg, 46.08 mol) in MeOH (5 mL) was added HCl/dioxane (4 M, 46.08 μL) under N₂. The suspension was degassed under vacuum and purged with H₂ several times. The mixture was stirred under H₂ (15 psi) at 20° C. for 2 h. The mixture was filtered and concentrated under reduced pressure to give the residue. The residue was purified by column chromatography (SiO₂, Dichloromethane:Methanol=1/0 to 10/1). Tert-butyl 2-(4-aminophenyl)-3-[[4-methyl-3-(trifluoromethyl)phenyl]carbamoyl]-1,2,3,4,4a,5,7,7a-octahydropyrrolo[3,4-b]pyridine-6-carboxylate (100 mg, crude) was obtained as a light yellow oil. LC-MS: (ES) m/z 519.3 (M+H⁺).

Step b) To a mixture of tert-butyl 2-(4-aminophenyl)-3-[[4-methyl-3-(trifluoromethyl)phenyl] carbamoyl]-1,2,3,4,4a,5,7,7a-octahydropyrrolo[3,4-b]pyridine-6-carboxylate (40 mg, 77.14 mol) and cyclopentanone (7.14 mg, 84.85 mol, 7.51 μL) in MeOH (2 mL) was added NaBH₃CN (14.54 mg, 231.41 mol) at 20° C. under N₂. Then AcOH (9.26 mg, 154.27 mol, 8.82 μL) was added to the mixture and the mixture was stirred at 20° C. for 10 h. The reaction mixture was partitioned between saturated NaHCO₃(aq) (20 ml) and DCM (20 mL). The organic phase was separated, dried filtered and concentrated under reduced pressure to give a residue. The residue was purified by prep-TLC (SiO₂, EtOAc:MeOH=50:1). Tert-butyl 2-[4-(cyclopentylamino)phenyl]-3-[[4-methyl-3-(trifluoromethyl)phen yl]carbamoyl]-1,2,3,4,4a,5,7,7a-octahydropyrrolo[3,4-b]pyridine-6-carboxylate (25 mg, crude) was obtained as a light yellow oil. LC-MS: (ES) m/z 587.3 (M+H⁺).

Step c) To a mixture of tert-butyl 2-[4-(cyclopentylamino)phenyl]-3-[[4-methyl-3-(trifluoromethyl)phenyl]carbamoyl]-1,2,3,4,4a,5,7,7a-octahydropyrrolo[3,4-b]pyridine-6-carboxylate (25 mg, 42.61 μmol), DIEA (11.01 mg, 85.22 mol, 14.84 μL) in DCM (1 mL) was added 2-fluoro-6-methyl-benzoyl chloride (7.35 mg, 42.61 mol) at 0° C. under N₂. The mixture was stirred at 0° C. for 1.5 h. Then the mixture was concentrated to get a crude product. The crude was purified by prep-HPLC (column: Venusil ASB Phenyl 150*30 mm*5 μm; mobile phase: [water (0.05% HCl)-ACN]; B %: 25%-55%, 12.5 min). Tert-butyl 2-[4-(cyclopentylamino)p henyl]-1-(2-fluoro-6-methyl-benzoyl)-3-[[4-methyl-3-(trifleoromethyl)phenyl]carbamoyl]-3,4,4a,5,7,7a-hexahydro-2H-pyrrolo[3,4-b]pyridine-6-carboxylate (15 mg, 19.76 umol, 17.85% yield, HCl) was obtained as a light yellow solid. LC-MS: (ES) m/z 723.4 (M+H⁺).

Step d) Tert-butyl 2-[4-(cyclopentylamino)phenyl]-1-(2-fluoro-6-methyl-benzoyl)-3-[[4-methyl-3-(trifluoromethyl)phenyl]carbamoyl]-3,4,4a,5,7,7a-hexahydro-2H-pyrrolo[3,4-b]pyridine-6-carboxylate (15 mg, 20.75 mol) in HCl/dioxane (4 M, 15.00 mL) was stirred at 20° C. for 2 h. Then the mixture was concentrated to get a crude product and purified by prep-HPLC (column: Venusil ASB Phenyl 150*30 mm*5 μm; mobile phase: [water (0.05% HCl)-ACN]; B %: 25%-55%, 12 min). 2-[4-(cyclopentylamino)phenyl]-1-(2-fluoro-6-methyl-benzoyl)-N-[4-methyl-3-(trifluoromethyl)phenyl]-2,3,4,4a,5,6,7,7a-octahydropyrrolo[3,4-b]pyridine-3-carboxamide (7 mg, 10.62 mol, 43.75% yield, 100% purity, HCl) was obtained as a light yellow solid. ¹H NMR (400 MHz, METHANOL-d₄) δ ppm 1.33 (br s, 4H), 1.50 (br d, J=6.08 Hz, 2H), 1.87-2.08 (m, 2H), 2.26 (d, J=13.83 Hz, 3 H), 2.33-2.51 (m, 5H), 2.55-2.69 (m, 1H), 2.92 (br s, 1H), 3.07-3.17 (m, 1H), 3.48-3.65 (m, 1H), 3.76-3.84 (m, 1H), 3.84-3.99 (m, 2H), 4.33 (br s, 1H), 4.78 (br s, 1H), 6.23-6.38 (m, 1H), 6.44-6.62 (m, 1H), 6.68-6.79 (m, 1H), 7.23-7.39 (m, 3H), 7.40-7.53 (m, 3H), 7.64-7.90 (m, 1H). LC-MS: (ES) m/z 623.3 (M+H⁺).

### Example S164: Synthesis of ((2R,3S)-3-(5-(tert-butyl)benzo[d]oxazol-2-yl)-2-(4-(cyclopentyl-amino)phenyl) piperidin-1-yl)(2-fluoro-6-methylphenyl)methanone (Compound No. 181)

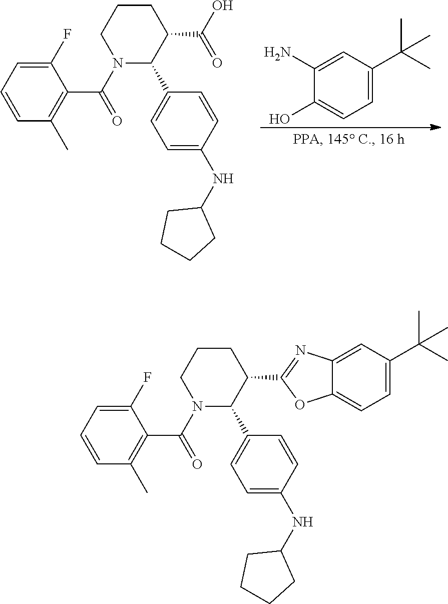 [SMILES: Cc1cccc(F)c1C(=O)N1CCC[C@H](C(=O)O)[C@@H]1c1ccc(NC2CCCC2)cc1 ; CC(C)(C)c1ccc(O)c(N)c1 ; Cc1cccc(F)c1C(=O)N1CCC[C@H](c2nc3cc(C(C)(C)C)ccc3o2)[C@@H]1c1ccc(NC2CCCC2)cc1]

To a mixture of (2R,3S)-2-[4-(cyclopentylamino)phenyl]-1-(2-fluoro-6-methyl-benzoyl) piperidine-3-carboxylic acid (500 mg, 1.18 mmol) and 2-amino-4-tert-butyl-phenol (389.23 mg, 2.36 mmol) in PPA (3 mL). The mixture was stirred at 145° C. for 16 h. The reaction mixture was cooled to 20° C., basified with saturated NaHCO₃ solution, then the mixture was extracted with ethyl acetate (50 mL×2). The combined organic phase was washed with brine (50 mL), dried with anhydrous MgSO₄ and was filtered. The filtrate was evaporated under vacuum to give a residue. The residue was purified by column chromatography (SiO₂, petroleum ether/ethyl acetate=100/0 to 3:1). The result product was purified by prep-HPLC (column: Agela ASB 150*25 mm*5 μm; mobile phase: [water (0.05% HCl)-ACN]; B %: 55%-85%, 8 min) to give [(2R,3S)-3-(5-tert-butyl-1,3-benzoxazol-2-yl)-2-[4-(cyclopentylamino)phenyl]-1-piperidyl]-(2-fluoro-6-methyl-phenyl)-methanone (45 mg, 80.46 mol, 6.83% yield, 99% purity) as a white solid. ¹H NMR (400 MHz, CDCl₃) δ 1.36 (9 H, s), 1.46 (1H, br d, J=6.60 Hz), 1.55 (3 H, br s), 1.80-1.99 (9H, m), 2.02 (3 H, s), 2.30-2.45 (2H, m), 3.06-3.23 (1H, m), 3.41 (1H, br d, J=12.47 Hz), 3.74 (1H, br d, J=6.36 Hz), 3.97-4.11 (1H, m), 6.74-6.85 (1H, m), 6.87-7.00 (2H, m), 7.14-7.23 (1H, m), 7.33-7.39 (1H, m), 7.39-7.44 (1H, m), 7.44-7.55 (2H, m), 7.59 (1 H, br s), 7.66-7.75 (1H, m). LCMS: m/z 554.4 (M+H⁺).

### Example S165: Synthesis of ((2R,3S)-3-(6-(tert-butyl)-1H-benzo[d]imidazol-2-yl)-2-(4-(cyclopentylamino)phenyl)piperidin-1-yl)(2-fluoro-6-methylphenyl)methanone (Compound No. 180)

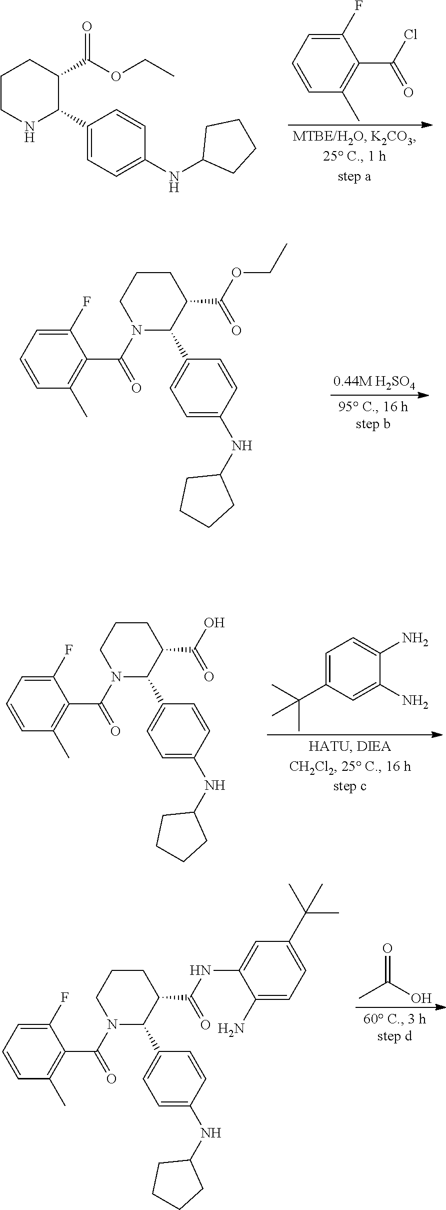

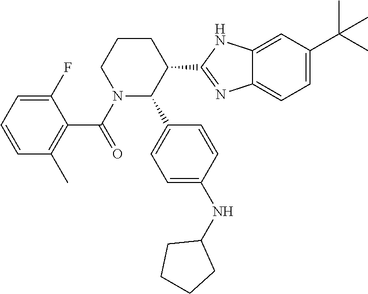 [SMILES: Cc1cccc(F)c1C(=O)N1CCC[C@H](c2nc3ccc(C(C)(C)C)cc3[nH]2)[C@@H]1c1ccc(NC2CCCC2)cc1]

Step a) K₂CO₃ (3.84 g, 27.81 mmol) was dissolved in H₂O (30 mL). After cooling to 25° C., MTBE (40 mL) was added, then ethyl(2R,3S)-2-[4-(cyclopentylamino)phenyl]piperidine-3-carboxylate (4.4 g, 13.90 mmol, L-DTTA) was added. The mixture was stirred at 25° C. for 0.5 h. 2-fluoro-6-methyl-benzoyl chloride (719.92 mg, 4.17 mmol) was dissolved in MTBE (40 mL) and added to the mixture dropwise. Then the mixture was stirred at 25° C. for 0.5 h. The reaction mixture was extracted with MTBE (40 mL*2), the combined organic phase was washed with brine (40 mL) and dried with anhydrous MgSO₄. Then the mixture was filtered. The filtrate was evaporated under vacuum to give a residue. The residue was purified by column chromatography (SiO₂, petroleum ether/ethyl acetate=100/0 to 3:1) (petroleum ether:ethyl acetate=3:1) to obtain ethyl(2R,3S)-2-[4-(cyclopentylamino)phenyl]-1-(2-fluoro-6-methyl-benzoyl) piperidine-3-carboxylate (1.7 g, 3.76 mmol, 27.02% yield) as a white solid. LC-MS: (ES) m/z 453.3 (M+H⁺).

Step b) The ethyl (2R,3S)-2-[4-(cyclopentylamino)phenyl]-1-(2-fluoro-6-methyl-benzoyl)piperidine-3-carboxylate (1.7 g, 3.76 mmol) was added to H₂SO₄ (0.44 M, 15.39 mL). The mixture was stirred at 95° C. for 16 h. The reaction mixture was cooled to 20° C., basified with saturated NaHCO₃ solution, then the mixture was extracted with ethyl acetate (50 mL*2). The combined organic phase was washed with brine (50 mL), dried with anhydrous MgSO₄ and was filtered. The filtrate was evaporated under vacuum to give (2R,3S)-2-[4-(cyclopentylamino)phenyl]-1-(2-fluoro-6-methyl-benzoyl)piperidine-3-carboxylic acid (1.5 g, 3.22 mmol, 85.60% yield, 91% purity) as a white solid. LCMS: m/z 425.2 (M+H⁺).

Step c) To a mixture of (2R,3S)-2-[4-(cyclopentylamino)phenyl]-1-(2-fluoro-6-methyl-benzoyl) piperidine-3-carboxylic acid (700 mg, 1.50 mmol) and 4-tert-butylbenzene-1,2-diamine (246.46 mg, 1.50 mmol) in DCM (10 mL) was added HATU (570.56 mg, 1.50 mmol) and DIEA (775.75 mg, 6.00 mmol, 1.05 mL). The mixture was stirred at 25° C. for 16 h. The reaction mixture was concentrated under reduced pressure to give a residue. The residue was purified by column chromatography (SiO₂, petroleum ether/ethyl acetate=100/0 to 1:1) to obtain (2R,3S)—N-(2-amino-5-tert-butyl-phenyl)-2-[4-(cyclopentylamino)phenyl]-1-(2-fluoro-6-methyl-benzoyl)piperidine-3-carboxamide (0.8 g, 981.19 mol, 65.39% yield, 70% purity) as a brown oil. LCMS: m/z 571.3 (M+H⁺).

Step d) The (2R,3S)—N-(2-amino-5-tert-butyl-phenyl)-2-[4-(cyclopentylamino)phenyl]-1-(2-fluoro-6-methyl-benzoyl)piperidine-3-carboxamide (800 mg, 981.19 mol) was dissolved in CH₃COOH (11.76 g, 195.83 mmol, 11.20 mL). The solution was stirred at 60° C. for 3 h. The solvent was evaporated under vacuum to give crude product. The crude product was purified by prep-HPLC (column: Agela ASB 150*25 mm*m; mobile phase: [water (0.05% HCl)-ACN]; B %: 32%-62%, 8 min) to give [(2R,3S)-3-(6-tert-butyl-1H-benzimida zol-2-yl)-2-[4-(cyclopentylamino)phenyl]-1-piperidyl]-(2-fluoro-6-methyl-phenyl)methanone (35 mg, 63.32 mol, 6.45% yield, 100% purity) as a white solid.

¹H NMR (400 MHz, DMSO-d₆) δ 1.25 (2 H, s), 1.30 (10H, d, J=3.42 Hz), 1.43 (7H, br d, J=10.52 Hz), 1.56-1.77 (7H, m), 1.89-2.02 (3H, m), 2.34 (3 H, s), 2.52-2.66 (2H, m), 3.96-4.10 (1H, m), 6.59 (1 H, br t, J=6.11 Hz), 7.03-7.10 (2H, m), 7.15-7.22 (2H, m), 7.27-7.37 (2H, m), 7.54-7.60 (2H, m), 7.61-7.67 (1H, m). LCMS: m/z 553.4 (M+H⁺).

### Example S166: Synthesis of (2R,3S)-2-(4-(cyclopentylamino)phenyl)-1-(2-fluoro-6-methylbenzyl)-N-(4-methyl-3-(trifluoromethyl)phenyl)piperidine-3-carboxamide (Compound No. 159)

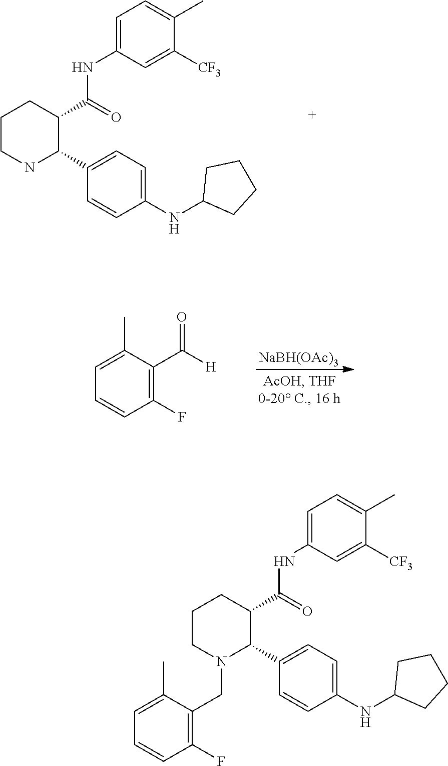

To a solution of (2R,3S)-2-[4-(cyclopentylamino)phenyl]-N-[4-methyl-3-(trifluoro methyl)phenyl]piperidine-3-carboxamide (100 mg, 224.46 mol) and 2-fluoro-6-methyl-benzaldehyde (46.51 mg, 336.69 mol) in THF (5 mL) was added NaBH(OAc)₃ (95.14 mg, 448.91 mol) and AcOH (13.48 mg, 224.46 mol, 12.84 μL) at 0° C. The mixture was stirred at 20° C. for 16 hr. The reaction mixture was quenched by addition aqueous NaHCO₃ (10 mL), and then extracted with DCM (30 mL*2). The combined organic layers were dried over anhydrous Na₂SO₄, filtered and concentrated under reduced pressure to give a residue. The residue was purified by prep-HPLC (HCl condition, column: Agela ASB 150*25 mm*5 μm; mobile phase: [water (0.05% HCl)-ACN]; B %: 55%-85%, 8 min) to give (2R,3S)-2-[4-(cyclopentylamino) phenyl]-1-[(2-fluoro-6-methyl-phenyl)methyl]-N-[4-methyl-3-(trifluoromethyl)phenyl]piperidine-3-carboxamide (80 mg, 140.93 mol, 62.79% yield, 100% purity) as a light yellow solid. ¹H NMR (400 MHz, METHANOL-d₄) δ 1.65 (br s, 4H), 1.80 (br s, 2H), 1.98 (br d, J=9.29 Hz, 3H), 2.18-2.37 (m, 3H), 2.41 (s, 3H), 2.45 (s, 3H), 3.26-3.30 (m, 1H), 3.41-3.53 (m, 1H), 3.55-3.63 (m, 1H), 3.87-3.96 (m, 1H), 4.21 (br d, J=13.69 Hz, 1H), 4.33-4.41 (m, 1H), 5.01 (br s, 1H), 7.10-7.20 (m, 2H), 7.27-7.46 (m, 4H), 7.69 (br dd, J=15.28, 8.44 Hz, 3H), 7.91 (s, 1H). LC-MS: (ES) m/z 568.3 (M+H⁺).

### Example S167: Synthesis of ((2R,3R)-2-(4-(cyclopentylamino)phenyl)-3-(((4-methyl-3-(trifluoro-methyl)phenyl)amino)methyl)piperidin-1-yl)(2-fluoro-6-methylphenyl)methanone (Compound No. 160)

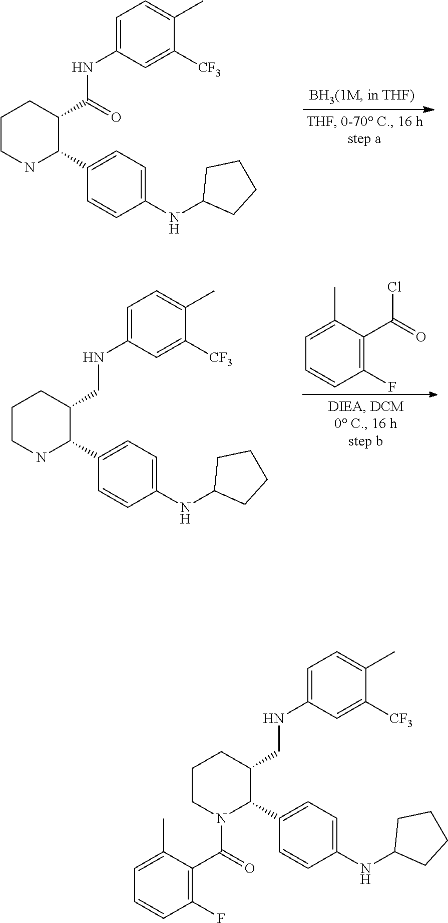 [SMILES: Cc1ccc(NC(=O)[C@H]2CCCN[C@H]2c2ccc(NC3CCCC3)cc2)cc1C(F)(F)F ; Cc1ccc(NC[C@H]2CCCN[C@H]2c2ccc(NC3CCCC3)cc2)cc1C(F)(F)F ; Cc1cccc(F)c1C(=O)Cl ; Cc1ccc(NC[C@H]2CCCN(C(=O)c3c(C)cccc3F)[C@H]2c2ccc(NC3CCCC3)cc2)cc1C(F)(F)F]

Step a) To a solution of (2R,3S)-2-[4-(cyclopentylamino)phenyl]-N-[4-methyl-3-(trifluoromethyl)phenyl]piperidine-3-carboxamide (200 mg, 448.91 mol) in THF (10 mL) was added BH₃ (in THF) (1 M, 1.80 mL) at 0° C. under N₂ atmosphere. The mixture was stirred under N₂ at 70° C. for 16 h. The reaction mixture was quenched by addition MeOH 10 mL, and then 3N HCl was added, stirred and refluxed for 1 h, then diluted with aqueous NaHCO₃ (20 mL) and extracted with ethyl acetate (40 mL*2). The combined organic layers were dried over anhydrous Na₂SO₄, filtered and concentrated under reduced pressure to give a residue. The residue was purified by prep-HPLC (neutral condition, column: Waters Xbridge Prep OBD C18 150*40 mm*10 m; mobile phase: [water (0.04% NH₃·H₂O+10 mM NH₄HCO₃)-ACN]; B %: 60%-90%, 10 min) to give N-[[(2R,3R)-2-[4-(cyclopentylamino)phenyl]-3-piperidyl]methyl]-4-methyl-3-(trifluoromethyl)aniline (50 mg, 114.71 mol, 25.55% yield, 99% purity) as a light yellow gum. LC-MS: (ES) m/z 432.3 (M+H⁺).

Step b) To a solution of N-[[(2R,3R)-2-[4-(cyclopentylamino)phenyl]-3-piperidyl]-methyl]-4-methyl-3-(trifluoromethyl)aniline (50 mg, 115.87 μmol) in DCM (2 mL) was added DIEA (29.95 mg, 231.73 mol, 40.36 μL) and then 2-fluoro-6-methyl-benzoyl chloride (20.00 mg, 115.87 mol, 1 eq) in DCM (1 mL) was added by dropwise at 0° C. The mixture was stirred at 0° C. for 1 h. The reaction mixture was quenched by addition H₂O (1 mL), and then extracted with DCM 20 mL (10 mL*2). The combined organic layers were dried over anhydrous Na₂SO₄, filtered and concentrated under reduced pressure to give a residue. The residue was purified by prep-HPLC (HCl condition, column: Agela DuraShell C18 150*25 mm*5 μm; mobile phase: [water (0.05% HCl)-ACN]; B %: 55%-82%, 7 min) to give [(2R,3R)-2-[4-(cyclopentylamino)phenyl]-3-[[4-methyl-3-(trifluoromethyl)anilino]methyl]-1-piperidyl]-(2-fluoro-6-methyl-phenyl)methanone (35 mg, 55.04 mol, 47.50% yield, 95% purity, HCl) as a white solid. ¹H NMR (400 MHz, METHANOL-d₄) δ 1.69-1.77 (m, 4H), 1.87 (s, 2H), 1.85-1.89 (m, 1H), 1.97-2.06 (m, 4 H), 2.39 (br d, J=17.85 Hz, 5H), 2.48-2.62 (m, 1H), 3.07-3.26 (m, 2H), 3.33-3.46 (m, 2 H), 3.99 (quin, J=6.91 Hz, 1H), 6.21 (dd, J=8.93, 6.24 Hz, 1H), 6.87-7.07 (m, 2H), 7.09-7.24 (m, 2H), 7.26-7.44 (m, 4H), 7.52 (t, J=8.44 Hz, 2H), 7.84 (dd, J=16.87, 8.56 Hz, 2H). LC-MS: (ES) m/z 568.4 (M+H⁺).

### Example S168: Synthesis of cis-3-(4-(cyclopentylamino)phenyl)-4-(2-fluoro-6-methylbenzoyl)-N-(4-methyl-3-(trifluoromethyl)phenyl)morpholine-2-carboxamide (Compound No. 187)

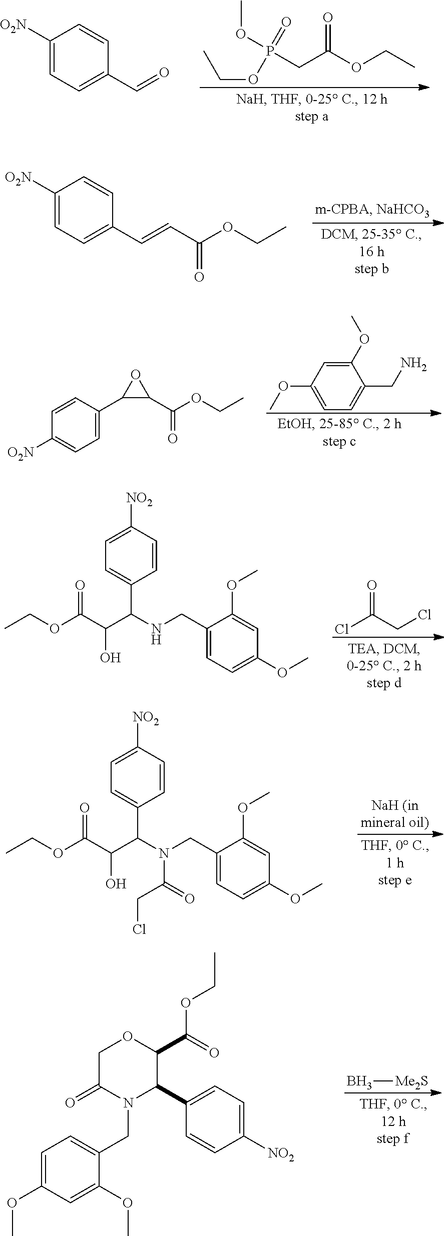

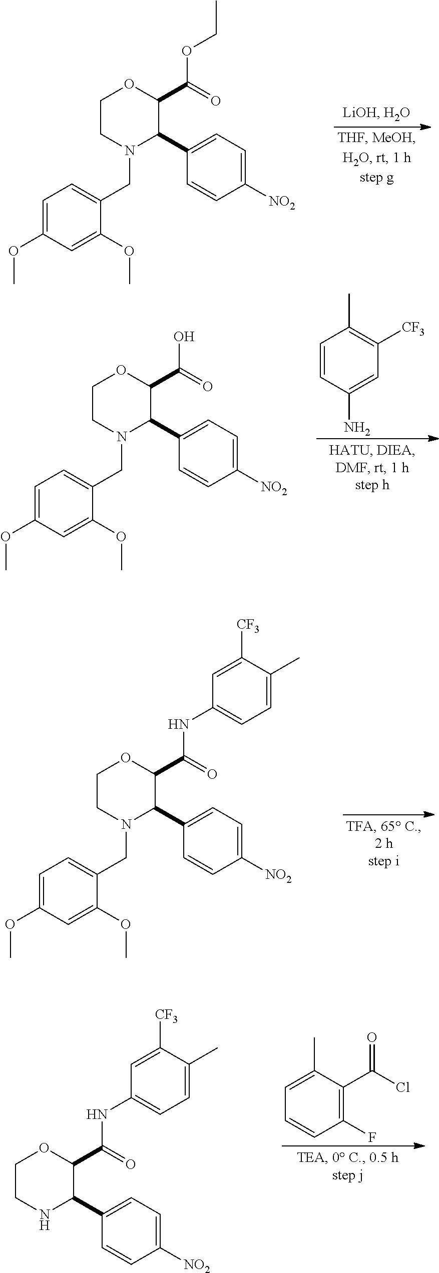 [SMILES: CCOC(=O)[C@@H]1OCCN(Cc2ccc(OC)cc2OC)[C@@H]1c1ccc([N+](=O)[O-])cc1 ; COc1ccc(CN2CCO[C@@H](C(=O)O)[C@H]2c2ccc([N+](=O)[O-])cc2)c(OC)c1 ; Cc1ccc(N)cc1C(F)(F)F ; COc1ccc(CN2CCO[C@@H](C(=O)Nc3ccc(C)c(C(F)(F)F)c3)[C@H]2c2ccc([N+](=O)[O-])cc2)c(OC)c1 ; Cc1ccc(NC(=O)[C@@H]2OCCN[C@@H]2c2ccc([N+](=O)[O-])cc2)cc1C(F)(F)F ; Cc1cccc(F)c1C(=O)Cl]

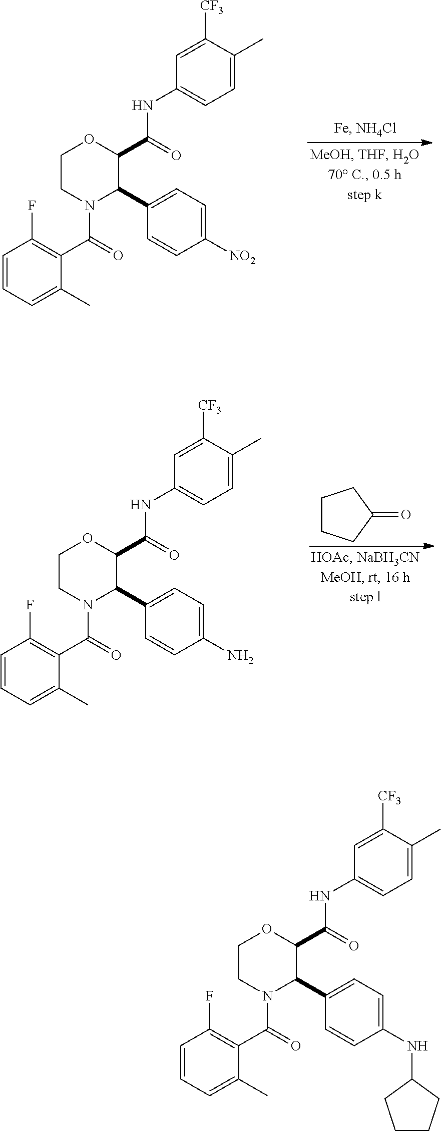 [SMILES: Cc1ccc(NC(=O)[C@@H]2OCCN(C(=O)c3c(C)cccc3F)[C@@H]2c2ccc([N+](=O)[O-])cc2)cc1C(F)(F)F ; Cc1ccc(NC(=O)[C@@H]2OCCN(C(=O)c3c(C)cccc3F)[C@@H]2c2ccc(N)cc2)cc1C(F)(F)F ; O=C1CCCC1 ; Cc1ccc(NC(=O)[C@@H]2OCCN(C(=O)c3c(C)cccc3F)[C@@H]2c2ccc(NC3CCCC3)cc2)cc1C(F)(F)F]

Step a) To a mixture of NaH (3.97 g, 99.26 mmol, 60% purity) in THF (200 mL) was added a solution of 4-nitrobenzaldehyde (10 g, 66.17 mmol) in THF (50 mL) slowly at 0° C. The reaction mixture was stirred at 0° C. for 20 min. Ethyl 2-diethoxyphosphorylacetate (14.83 g, 66.17 mmol, 13.13 mL) was added in small portions. The reaction mixture was stirred at 25° C. for another 12 hr. The reaction mixture was quenched by addition of NH₄Cl at 25° C., and then diluted with water (50 mL) and extracted with EtOAc 300 mL (100 mL×3). The combined organic layers were dried over Na₂SO₄, filtered and concentrated under reduced pressure to give the residue. The residue was purified by column chromatography (SiO₂, petroleum ether/ethyl acetate=100/0 to 10:1) to give ethyl (E)-3-(4-nitrophenyl)prop-2-enoate (7 g, 31.64 mmol, 47.82% yield) as a light yellow solid. ¹H NMR (400 MHz, DMSO-d₆): δ 1.24 (t, J=7.1 Hz, 3H), 4.18 (q, J=7.1 Hz, 2H), 6.82 (d, J=16.1 Hz, 1H), 7.72 (d, J=16.1 Hz, 1H), 7.98 (d, J=8.8 Hz, 2H), 8.20 (d, J=9.0 Hz, 2H).

Step b) To a solution of ethyl (E)-3-(4-nitrophenyl)prop-2-enoate (7 g, 31.64 mmol) in DCM (160 mL) was added NaHCO₃ (saturated aqueous solution) (160 mL) and m-CPBA (20.48 g, 94.93 mmol, 80% purity) at 25° C. Then the mixture was stirred at 35° C. for 16 h. The mixture was quenched by addition of saturated Na₂S₂O₃ solution (150 mL) and extracted with DCM (3×50 mL), and the combined organic layers was dried, filtered and concentrated in vacuo to give the crude. The crude was purified by flash silica gel chromatography (ISCO®; 40 g SepaFlash® Silica Flash Column, eluent of 0˜10% ethyl acetate/petroleum ether gradient@40 mL/min) to give ethyl 3-(4-nitrophenyl)oxirane-2-carboxylate (4 g, 16.86 mmol, 53.29% yield) as yellow gum. ¹H NMR (400 MHz, CDCl₃) δ 1.35 (t, J=7.2 Hz, 3H), 3.50 (d, J=1.5 Hz, 1H), 4.21 (d, J=1.5 Hz, 1H), 4.25-4.39 (m, 2H), 7.49 (d, J=8.5 Hz, 2H), 8.19-8.28 (m, 2H).

Step c) To a solution of ethyl 3-(4-nitrophenyl)oxirane-2-carboxylate (0.8 g, 3.37 mmol) in EtOH (4 mL) was added (2,4-dimethoxyphenyl)methanamine (563.91 mg, 3.37 mmol, 508.03 μL) at 25° C. Then the mixture was stirred at 85° C. for 12 h. The mixture was concentrated in vacuo to give the crude. The crude was purified by flash silica gel chromatography (ISCO®; 40 g SepaFlash® Silica Flash Column, eluent of 0˜30% ethyl acetate/petroleum ether gradient@45 mL/min) to give ethyl 3-[(2,4-dimethoxyphenyl)methylamino]-2-hydroxy-3-(4-nitrophenyl) propanoate (0.3 g, 741.82 mol, 22.00% yield) as light yellow solid. LC-MS: (ES) m/z 405.2 (M+H⁺).

Step d) To a solution of ethyl 3-[(2,4-dimethoxyphenyl) methylamino]-2-hydroxy-3-(4-nitro phenyl)propanoate (0.3 g, 741.82 mol) and TEA (82.57 mg, 816.00 mol, 113.58 μL) in DCM (5 mL) was added 2-chloroacetyl chloride (83.78 mg, 741.82 mol, 59.00 μL) at 0° C. Then the mixture was stirred at 25° C. for 2 h. The mixture was diluted with DCM (20 mL), washed with H₂O (2×20 mL), brine (2×20 mL), dried, filtered and concentrated in vacuo to give the crude ethyl 3-[(2-chloroacetyl)-[(2,4-dimethoxyphenyl) methyl] amino]-2-hydroxy-3-(4-nitro phenyl) propanoate (0.36 g, crude) as light yellow gum. The crude product was used directly in the next step without further purification. LC-MS: (ES) m/z 481.2 (M+H⁺).

Step e) To a solution of ethyl 3-[(2-chloroacetyl)-[(2,4-dimethoxyphenyl) methyl]amino]-2-hydroxy-3-(4-nitrophenyl) propanoate (0.36 g, 748.60 mol) in THF (30 mL) was added NaH (60% dispension in mineral oil) (31 mg, 775.07 mol, 60% purity) at 0° C. Then the mixture was stirred at 0° C. for 1 h. The mixture was poured into saturated NH₄Cl solution (30 mL) carefully and extracted with EtOAc (2×20 mL). The combined organic phase separated was washed with brine, dried, filtered and concentrated in vacuo to give the crude. The crude was purified by flash silica gel chromatography (ISCO®; 12 g SepaFlash® Silica Flash Column, eluent of 0˜60% ethyl acetate/petroleum ether gradient@20 mL/min) to give cis-ethyl 4-[(2,4-dimethoxyphenyl)methyl]-3-(4-nitrophenyl)-5-oxo-morpholine-2-carboxylate (0.19 g, 427.51 mol, 57.11% yield) as light yellow gum. ¹H NMR (400 MHz, CDCl₃) δ 1.07 (t, J=7.1 Hz, 3H), 3.76 (s, 3H), 3.79-3.87 (m, 4H), 3.93-4.09 (m, 2H), 4.42 (d, J=16.9 Hz, 1H), 4.64 (d, J=3.4 Hz, 1H), 4.68 (d, J=16.9 Hz, 1H), 4.86 (d, J=3.4 Hz, 1H), 5.01 (d, J=14.4 Hz, 1H), 6.39 (d, J=2.2 Hz, 1H), 6.46 (dd, J=8.3, 2.2 Hz, 1 H), 7.20 (d, J=8.3 Hz, 1H), 7.39 (d, J=8.6 Hz, 2H), 8.18 (d, J=8.8 Hz, 2H). LC-MS: (ES) m z 445.2 (M+H⁺).

Step f) To a solution of cis-ethyl 4-[(2,4-dimethoxyphenyl) methyl]-3-(4-nitrophenyl)-5-oxo-morpholine-2-carboxylate (0.16 g, 360.01 mol) in THF (2 mL) was added BH₃-Me₂S (10 M, 108.00 μL) at 0° C. Then the mixture was stirred at 25° C. for 12 h. The mixture was quenched with H₂O (10 mL) carefully and extracted with EtOAc (2×20 mL). The combined organic phase separated was washed with brine, dried, filtered and concentrated in vacuo to give the crude. The crude was purified by flash silica gel chromatography (ISCO®; 4 g SepaFlash® Silica Flash Column, eluent of 0˜50% ethyl acetate/petroleum ether gradient@20 mL/min) to give the target product cis-ethyl 4-[(2,4-dimethoxyphenyl)methyl]-3-(4-nitrophenyl)morpholine-2-carboxylate (125 mg, 290.39 mol, 80.66% yield) as light yellow gum. ¹H NMR (400 MHz, acetonitrile-d₃) δ 0.88 (t, J=7.1 Hz, 3H), 2.42 (d, J=12.7 Hz, 1H), 2.72-2.81 (m, 1H), 3.15 (d, J=13.4 Hz, 1H), 3.49 (d, J=13.7 Hz, 1H), 3.74 (s, 3H), 3.75-3.82 (m, 4H), 3.86 (q, J=7.1 Hz, 2H), 4.14-4.25 (m, 2H), 4.61 (d, J=3.7 Hz, 1H), 6.50-6.57 (m, 2H), 7.31 (d, J=8.1 Hz, 1H), 7.70 (d, J=8.6 Hz, 2H), 8.17 (d, J=8.8 Hz, 2H). LC-MS: (ES) m/z 431.2 (M+H⁺).

Step g) To a solution of cis-ethyl 4-[(2,4-dimethoxyphenyl)methyl]-3-(4-nitrophenyl) morpholine-2-carboxylate (417 mg, 968.75 mol) in THF (2.5 mL)/MeOH (2.5 mL)/H₂O (1 mL) was added LiOH·H₂O (60.98 mg, 1.45 mmol, 726.57 μL). Then the mixture was stirred at 25° C. for 1 h. The mixture was quenched with brine (1 mL) carefully and acidified to pH=4-5 by addition of HCl (2 M), then extracted with EtOAc (5×10 mL). The combined organic phase separated was washed with brine (3×5 mL), dried, filtered and concentrated in vacuo to give cis-4-[(2,4-dimethoxyphenyl)methyl]-3-(4-nitrophenyl) morpholine-2-carboxylic acid (380 mg, 944.34 mol, 97.48% yield) as yellow solid. ¹H NMR (400 MHz, acetonitrile-d₃) δ 2.73 (br d, J=13.1 Hz, 1H), 3.03 (t, J=10.5 Hz, 1H), 3.45 (br d, J=13.1 Hz, 1H), 3.70 (s, 3H), 3.74-3.83 (m, 4H), 3.97-4.06 (m, 1H), 4.14-4.23 (m, 1H), 4.64 (br s, 1H), 5.02 (br s, 1H), 6.51 (d, J=2.3 Hz, 1H), 6.55 (dd, J=8.3, 2.5 Hz, 1 H), 7.49 (d, J=8.5 Hz, 1H), 7.86 (br d, J=8.5 Hz, 2H), 8.23 (d, J=9.0 Hz, 2H).

Step h) To a solution of cis-4-[(2,4-dimethoxyphenyl)methyl]-3-(4-nitrophenyl)morpholine-2-carboxylic acid (385 mg, 956.77 mol), 4-methyl-3-(trifluoromethyl)aniline (217.85 mg, 1.24 mmol, 178.57 μL) and DIEA (370.97 mg, 2.87 mmol, 499.95 μL) in DMF (5 mL) was added HATU (545.69 mg, 1.44 mmol). Then the mixture was stirred at 25° C. for 1 h. The mixture was quenched with H₂O (3 mL) carefully and extracted with EtOAc (2×10 mL). The combined organic phase separated was washed with brine, dried, filtered and concentrated in vacuo to give the crude product. The crude product was purified by prep-TLC to give cis-4-[(2,4-dimethoxyphenyl) methyl]-N-[4-methyl-3-(trifluoromethyl) phenyl]-3-(4-nitrophenyl) morpholine-2-carboxamide (510 mg, 911.48 umol, 95.27% yield) as light yellow gum. ¹H NMR (400 MHz, acetonitrile-d₃) δ 2.36 (d, J=1.2 Hz, 3H), 2.48 (dd, J=12.8, 2.3 Hz, 1H), 2.79-2.86 (m, 1H), 3.11 (d, J=13.7 Hz, 1 H), 3.58 (d, J=13.7 Hz, 1H), 3.73 (s, 3H), 3.80 (s, 3H), 3.91 (td, J=11.6, 3.2 Hz, 1H), 4.24 (dd, J=11.4, 3.1 Hz, 1H), 4.34 (d, J=3.4 Hz, 1H), 4.62 (d, J=3.7 Hz, 1H), 6.50-6.57 (m, 2 H), 7.21 (d, J=8.1 Hz, 1H), 7.34 (d, J=8.1 Hz, 1H), 7.41-7.51 (m, 1H), 7.64-7.76 (m, 3 H), 8.12 (d, J=8.8 Hz, 2H), 8.77 (s, 1H). LC-MS: (ES) m/z 560.2 (M+H⁺).

Step i) A mixture of cis-4-[(2,4-dimethoxyphenyl) methyl]-N-[4-methyl-3-(trifluoromethyl) phenyl]-3-(4-nitrophenyl)morpholine-2-carboxamide (0.19 g, 339.57 mol) in TFA (4 mL) was stirred at 65° C. for 2 h. The mixture was diluted with DCM (6 mL) and alkalified to pH=8-9 by addition of saturated NaHCO₃ solution. The organic layer was separated to give a solution of cis-N-[4-methyl-3-(trifluoromethyl)phenyl]-3-(4-nitrophenyl)morpholine-2-carboxamide (assumed in quantitative yield (139.01 mg) obtained) in DCM (6 mL), and the organic phase was used directly in the next step.

Step j) To a solution of cis-N-[4-methyl-3-(trifluoromethyl) phenyl]-3-(4-nitrophenyl) morpholine-2-carboxamide (139.01 mg, 339.58 mol) in DCM (6 mL) was added TEA (51.54 mg, 509.37 umol, 70.90 μL) and 2-fluoro-6-methyl-benzoyl chloride (58.61 mg, 339.58 mol) at 0° C. Then the mixture was stirred at 0° C. for 0.5 h. The mixture was diluted with DCM (20 mL), washed with brine (2×10 mL), dried, filtered and concentrated in vacuo to give the crude. The crude was purified by flash silica gel chromatography (ISCO®; 4 g SepaFlash® Silica Flash Column, Eluent of 0˜10% MeOH/DCM gradient@18 mL/min) to give cis-4-(2-fluoro-6-methyl-benzoyl)-N-[4-methyl-3-(trifluoromethyl)phenyl]-3-(4-nitrophenyl)morpholine-2-carboxamide (160 mg, 269.85 mol, 79.47% yield, 92% purity) as light yellow solid. ¹H NMR (400 MHz, DMSO-d₆) δ 1.86-2.02 (m, 3H), 2.33-2.43 (m, 5H), 3.11-3.22 (m, 1H), 3.29-3.45 (m, 1H), 3.73-3.90 (m, 2H), 4.15-4.28 (m, 1H), 4.65-4.83 (m, 1H), 6.26 (br s, 1H), 6.96-7.19 (m, 3H), 7.28-7.40 (m, 2H), 7.68 (br d, J=7.78 Hz, 1H), 7.85 (d, J=2.01 Hz, 1H), 7.92 (dd, J=17.82, 8.78 Hz, 2H), 8.12-8.23 (m, 2H), 9.86 (br d, J=14.81 Hz, 1H). LC-MS Rₜ (retention time): 0.93 min; MS: (ES) m/z 546.2 (M+H⁺).

Step k) To a mixture of cis-4-(2-fluoro-6-methyl-benzoyl)-N-[4-methyl-3-(trifluoromethyl) phenyl]-3-(4-nitrophenyl)morpholine-2-carboxamide (120 mg, 219.99 mol) and NH₄Cl (11.77 mg, 219.99 mol) in MeOH (2.5 mL)/THF (2.5 mL)/H₂O (1 mL) was added Fe (73.71 mg, 1.32 mmol). Then the mixture was stirred at 70° C. for 0.5 h. The mixture was diluted with EtOAc (20 mL) and alkalified to pH=8-9 by addition of saturated NaHCO₃ solution. The organic phase separated was washed with brine, dried, filtered and concentrated in vacuo to give the crude cis-3-(4-aminophenyl)-4-(2-fluoro-6-methyl-benzoyl)-N-[4-methyl-3-(trifluoro methyl)phenyl]morpholine-2-carboxamide (100 mg, 180.41 mol, 82.01% yield, 93% purity) as light yellow solid. LC-MS: (ES) m/z 516.2 (M+H⁺).

Step 1) To a mixture of cis-3-(4-aminophenyl)-4-(2-fluoro-6-methyl-benzoyl)-N-[4-methyl-3-(trifluoromethyl)phenyl]morpholine-2-carboxamide (100 mg, 193.99 mol) in MeOH (3 mL) was added cyclopentanone (19.58 mg, 232.78 mol, 20.61 μL), HOAc (11.65 mg, 193.99 mol, 11.09 μL) and NaBH₃CN (30.48 mg, 484.97 mol) in one portion at 25° C. under N₂. The mixture was stirred at 25° C. for 16 h. The reaction mixture was quenched with H₂O (10 mL) and extracted with DCM (3×15 mL). The combined organic layers were washed with brine (2×5 mL), dried, filtered and concentrated in vacuo to give the crude. The crude was purified by prep-HPLC (column: Agela Durashell C18 150×30 5 u; mobile phase: [water (0.05% HCl)-ACN]; B %: 42%-72%, 8 min) to give the target product cis-3-[4-(cyclopentyl amino) phenyl]-4-(2-fluoro-6-methyl-benzoyl)-N-[4-methyl-3-(trifluoromethyl)phenyl]morpholine-2-carboxamide (0.1 g, 171.35 mol) (45 mg, 98% purity) as light yellow solid. ¹H NMR (400 MHz, CDCl₃) δ 1.29-1.48 (m, 3H), 1.63-1.74 (m, 3H), 1.87-2.00 (m, 2H), 2.01-2.09 (m, 3H), 2.35-2.49 (m, 4H), 3.12 (br d, J=13.80 Hz, 1H), 3.38-3.53 (m, 1H), 3.56-3.76 (m, 2H), 3.81-4.00 (m, 1H), 4.16 (br d, J=11.04 Hz, 1H), 4.35-4.49 (m, 1H), 4.52-4.58 (m, 1H), 4.63 (dd, J=14.05, 3.01 Hz, 1H), 4.92 (d, J=3.51 Hz, 1H), 6.36-6.42 (m, 1H), 6.50 (d, J=8.53 Hz, 1H), 6.82-6.99 (m, 2H), 6.99-7.16 (m, 2H), 7.18-7.25 (m, 1H), 7.32 (td, J=7.97, 6.15 Hz, 1H), 7.37-7.51 (m, 1H), 7.52-7.59 (m, 1H), 7.69 (br d, J=8.03 Hz, 1H), 8.13-8.45 (m, 1H). LC-MS: (ES) m z 584.3 (M+H⁺).

### BIOLOGICAL EXAMPLES

### Example B1: Inhibition of C5a-C5aR Binding

U937 cells were originally obtained from the American Type Culture Collection (ATCC) and transfected with human C5a receptor (C5aR). U937/C5aR cells were cultured at 37° C., 5% CO₂ in RPMI1640 (Gibco) supplemented with 10% FBS (Gibco) and 350 g/ml Geneticin (Gibco) and passaged every 3 days to maintain the density range from 1×105 to 2×10⁶ cells/ml.

Human C5a biotinylation was performed according to the procedure provided by manufacturer (Thermo Scientific, A39257). 10 mM solution of Sulfo-NHS-LC-Biotin was prepared by adding 180μl ultrapure H₂O to the 1 mg vial immediately. 12 μl 10 mM biotin reagent was added to 200 g human C5a solution, gently pipetted for 3 seconds, and incubated on ice for 2 hours. Amicon ultra-0.5 centrifuge filter device (Millipore, UFC5003BK) was pre-rinsed with Milli-Q H₂O, centrifuged at 14000 g for 5 min immediately before use. Up to 500 d sample (diluted by PBS) was added to the device and capped. The device was spinned at 14000 g for approximately 5 min. 250 μl PBS was added to the filter device, spinned at 14000 g for 5 min, and repeatedly washed 6 times. The filter device was separated from the microcentrifuge tube and placed upside down in a clean microcentrifuge tube, spinned for 2 min at 1000 g to transfer the concentrated sample from the device to the tube.

U937/C5aR cells were collected and washed twice by PBS, cells were suspended in PBS+0.1% BSA buffer at the density of 3×10⁶ cells/ml. 100 μl cell suspension was added to a 96 well microplate. 50 μl compound diluted in assay buffer and 50 μl biotinylated ligand human C5a (30 nM) were added to corresponding wells in order and the plate was incubated on ice for 120 min and then centrifuged at 1000 rpm for 3-5 min at 4° C. Supernatant was removed and cells were washed by pre-cold PBS twice. 100 μl FITC conjugated streptavidin was added to cells, incubated on ice for another 30 min, and then centrifuged at 1000 rpm for 3-5 min at 4° C. Supernatant was removed and cells were washed by pre-cold PBS twice. 150 μl PBS was added to suspend the cells and signals were detected by FACS (Beckman, Cytoflex). IC₅₀ values were calculated by GraphPad Prism software and provided in Table

TABLE B1

| Compound | IC₅₀ |
|---|---|
| No./name | (nM) |
| 2 | + |
| 4 | + |
| 5 | +++ |
| 6 | ++ |
| 7 | + |
| 8 | + |
| 9 | + |
| 10 | ++ |
| 11 | ++ |
| 13 | + |
| 14 | + |
| 15 | + |
| 16 | + |
| 17 | ++ |
| 18 | + |
| 19 | + |
| 20 | + |
| 22 | ++ |
| 23 | + |
| 24 | + |
| 25 | ++ |
| 26 | + |
| 27 | + |
| 28 | + |
| 29 | + |
| 30 | + |
| 32 | + |
| 33 | + |
| 34 | + |
| 35 | + |
| 36 | ++ |
| 38 | + |
| 40 | ++++ |
| 41 | ++ |
| 42 | ++++ |
| 44 | +++ |
| 46 | +++ |
| 47 | +++ |
| 49 | ++++ |
| 50 | ++++ |
| 51 | ++++ |
| 52 | + |
| 54 | +++ |
| 55 | +++ |
| 56 | ++++ |
| 57 | +++ |
| 58 | +++ |
| 59 | +++ |
| 60 | + |
| 61 | + |
| 62 | +++ |
| 63 | ++++ |
| 64 | ++ |
| 65 | +++ |
| 66 | ++++ |
| 67 | ++++ |
| 68 | + |
| 69 | +++ |
| 70 | ++ |
| 71 | ++++ |
| 73 | +++ |
| 74 | + |
| 76 | + |
| 77 | + |
| 78 | +++ |
| 79 | + |
| 83 | + |
| 84 | ++++ |
| 88 | +++ |
| 89 | ++++ |
| 90 | +++ |
| 92 | + |
| 94 | ++++ |
| 95 | ++ |
| 96 | ++++ |
| 97 | ++ |
| 98 | ++++ |
| 99 | + |
| 102 | + |
| 103 | + |
| 104 | + |
| 105 | + |
| 107 | + |
| 109 | ++ |
| 110 | +++ |
| 111 | +++ |
| 113 | + |
| 114 | ++++ |
| 115 | +++ |
| 118 | ++++ |
| 119 | ++++ |
| 121 | +++ |
| 122 | + |
| 123 | +++ |
| 124 | +++ |
| 126 | ++++ |
| 126 | ++++ |
| 129 | + |
| 134 | + |
| 135 | +++ |
| 136 | + |
| 138 | +++ |
| 139 | ++++ |
| 141 | +++ |
| 142 | + |
| 144 | +++ |
| 144 | ++ |
| 147 | ++++ |
| 155 | ++ |
| 158 | ++++ |
| 161 | +++ |
| 162 | +++ |
| 166 | + |
| 170 | ++ |
| 180 | + |
| 184 | + |
| 187 | +++ |
| 188 | ++++ |
| 189 | +++ |
| 190 | ++ |
| 200 | − |
| 201 | − |
| 202 | − |

−: IC₅₀ > 5000 nM in migration assay; or IC₅₀ > 10000 nM in Ca2+ flux assay
+: 5000 nM ≥ IC₅₀ ≥ 2000 nM in migration assay (compound shows weak activity at 2000 nM and the % inhibition is less than 50%) or 10000 nM ≥ IC₅₀ ≥ 2000 nM
++: 500 nM ≤ IC₅₀ < 2000 nM;
+++: 50 nM ≤ IC₅₀ < 500 nM;
++++: IC₅₀ < 50 nM.

### Example B2: Inhibition of C5a-C5aR Binding Determined by Cell Migration Assays

Migration assay was performed by using polycarbonate membrane with 3.0 m pore (Corning). U937/C5aR cells were collected and washed twice by PBS; cells were suspended in Hank's balanced salt solution (HBSS)+1% FBS buffer at the density of 6×106 cells/ml. Cells were premixed with compound and added to insert well, ligand human C5a and compound were added to bottom well in order, gently mix, incubate for 30 min at 37° C., 500 CO₂. Put insert plate into bottom well, migrate for 180 min at 37° C., 500 CO₂. Gently remove the insert well, add 50 μl CellTiter-Glo (Promega), gently shaking for 5 min at room temperature, transfer 150 μl mixture to black plate and read luminescence intensity by

### Confidential Draft

microplate reader (BioTek). IC50 value was calculated by GraphPad Prism software and provided in Table B2.

TABLE B2

| | Com- | |
|---|---|---|
| | pound | IC₅₀ |
| Compound Name | No. | (nM) |
| (2R,3S)-2-(4-(cyclopentylamino)phenyl)-N-(4-methyl-3- | 5 | +++ |
| (trifluoromethyl)phenyl)-1-(quinazolin-4-yl)piperidine-3- | | |
| carboxamide | | |
| (2R,3S)-2-(4-(cyclopentylamino)phenyl)-N-(4-methyl-3- | 188 | ++++ |
| (trifluoromethyl)phenyl)-1-(quinoline-8- | | |
| carbonyl)piperidine-3-carboxamide | | |
| (2R,3S)-2-(4-(cyclopentylamino)phenyl)-N-(4-methyl-3- | 162 | ++ |
| (trifluoromethyl)phenyl)-1-(pyrido[3,4-d]pyrimidin-4- | | |
| yl)piperidine-3-carboxamide | | |
| benzyl cyclopentyl(4-((2R,3S)-3-((4-methyl-3- | 164 | + |
| (trifluoromethyl)phenyl)carba moyl)-1-(1,7-naphthyridin- | | |
| 8-yl)piperidin-2-yl)phenyl)carbamate | | |
| (2R,3S)-2-(4-(cyclopentylamino)phenyl)-N-(4-methyl-3- | 37 | − |
| (trifluoromethyl)pheny l)-1- | | |
| ((perfluorophenyl)sulfonyl)piperidine-3-carboxamide | | |
| (2R,3S)-2-(4-(cyclopentyl(1,7-naphthyridin-8- | 168 | − |
| yl)amino)phenyl)-N-(4-methyl-3- | | |
| (trifluoromethyl)phenyl)piperidine-3-carboxamide | | |
| cis-1-(2-fluoro-6-methylbenzoyl)-N-(4-methyl-3- | 203 | ++ |
| (trifluoromethyl)phenyl)-2-(2-oxaspiro[4.5]decan-8- | | |
| yl)piperidine-3-carboxamide | | |
| cis-3-(4-aminophenyl)-4-(2-fluoro-6-methylbenzoyl)-N- | 187 | +++ |
| (4-methyl-3-(trifluoromethyl)phenyl)morpholine-2- | | |
| carboxamide | | |
| cis-4-(4-(cyclopentylamino)phenyl)-N-(4-methyl-3- | 43 | − |
| (trifluoromethyl)phenyl)-6-oxo-2,3,4,6,11,11a- | | |
| hexahydro-1H-pyrido[1,2-b]isoquinoline-3-carboxamide | | |
| (3S,4R)-4-(4-(cyclopentylamino)phenyl)-N-(4-methyl-3- | 44 | ++++ |
| (trifluoromethyl)phenyl)-6-oxo-1,2,3,4,6,11,12,12a- | | |
| octahydrobenzo[e]pyrido[1,2-a]azepine-3-carboxamide | | |
| cis-2-(4-(cyclopentylamino)phenyl)-1-(2-fluoro-6- | 193 | +++ |
| methylbenzoyl)-N-(4-methyl-3- | | |
| (trifluoromethyl)phenyl)octahydro-1H- | | |
| cyclopenta[b]pyridine-3-carboxamide | | |
| (2R,3S,4aR,7aR)-2-(4-(cyclopentylamino)phenyl)-1-(2- | 47 | ++++ |
| fluoro-6-methylbenzoyl)-N-(4-methyl-3- | | |
| (trifluoromethyl)phenyl)octahydro-1H- | | |
| cyclopenta[b]pyridine-3-carboxamide | | |
| cis-2-(4-(cyclopentylamino)phenyl)-1-(2-fluoro-6- | 118 | ++++ |
| methylbenzoyl)-N-(4-methyl-3- | | |
| (trifluoromethyl)phenyl)octahydrofuro[3,4-b]pyridine-3- | | |
| carboxamide | | |
| (2R,3S,4aR,7aS)-2-(4-(cyclopentylamino)phenyl)-1-(2- | 119 | ++++ |
| fluoro-6-methylbenzoy 1)-N-(4-methyl-3- | | |
| (trifluoromethyl)phenyl)octahydrofuro[3,4-b]pyridine-3- | | |
| carboxamide | | |
| cis-4-(4-(cyclopentylamino)phenyl)-7-fluoro-N-(4- | 41 | +++ |
| methyl-3-(trifluorometh yl)phenyl)-6-oxo- | | |
| 1,2,3,4,6,11,12,12a-octahydrobenzo[e]pyrido[1,2- | | |
| alazepine-3-carboxamide | | |
| (2R,3S,4aR,7aR)-2-(4-(cyclopentylamino)phenyl)-1-(2- | 54 | +++ |
| fluoro-6-methylbenzoyl)-N-(1-methyl-1H-pyrazol-4- | | |
| yl)octahydro-1H-cyclopenta[b]pyridine-3-carboxamide | | |
| cis-2-(4-(cyclopentylamino)phenyl)-1-(2-fluoro-6- | 77 | − |
| methylbenzoyl)-N-(2-methyl-1,2,3,4- | | |
| tetrahydroisoquinolin-6-yl)octahydro-1H- | | |
| cyclopenta[b]pyridine-3-carboxamide | | |
| cis-2-(4-(cyclopentylamino)phenyl)-1-(2-fluoro-6- | 57 | +++ |
| methylbenzoyl)-N-(pyridin-3-yl)octahydro-1H- | | |
| cyclopenta[b]pyridine-3-carboxamide | | |
| cis-N-(3-cyano-4-methylphenyl)-2-(4- | 62 | +++ |
| (cyclopentylamino)phenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-1-(2-fluoro-6- | 147 | +++ |
| methylbenzoyl)-N-(1-methyl-1H-indazol-5-yl)octahydro- | | |
| 1H-cyclopenta[b]pyridine-3-carboxamide | | |
| (2R,3S,4aR,7aR)-2-(4-(cyclopentylamino)phenyl)-1-(2- | 49 | ++++ |
| fluoro-6-methylbenzoyl)-N-(1-methyl-1H-indazol-5- | | |
| yl)octahydro-1H-cyclopenta[b]pyridine-3-carboxamide | | |
| cis-2-(4-(cyclopentylamino)phenyl)-1-(2-fluoro-6- | 66 | ++++ |
| methylbenzoyl)-N-(1-methyl-1H-indazol-6-yl)octahydro- | | |
| 1H-cyclopenta[b]pyridine-3-carboxamide | | |
| (2R,3S,4aR,7aR)-2-(4-(cyclopentylamino)phenyl)-1-(2- | 50 | ++++ |
| fluoro-6-methylbenzoyl)-N-(1-methyl-1H-indazol-6- | | |
| yl)octahydro-1H-cyclopenta[b]pyridine-3-carboxamide | | |
| cis-N-(benzo[d]oxazol-6-yl)-2-(4- | 67 | +++ |
| (cyclopentylamino)phenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-1-(2-fluoro-6- | 46 | +++ |
| methylbenzoyl)-N-(3-(trifluoromethyl)phenyl)octahydro- | | |
| 1H-cyclopenta[b]pyridine-3-carboxamide | | |
| cis-2-(4-(cyclopentylamino)phenyl)-1-(2-fluoro-6- | 70 | ++ |
| methylbenzoyl)-N-(3-(methyls ulfonyl)phenyl)octahydro- | | |
| 1H-cyclopenta[b]pyridine-3-carboxamide | | |
| cis-2-(4-(cyclopentylamino)phenyl)-N-(4-methyl-3- | 111 | +++ |
| (trifluoromethyl)phenyl)-1-(tetrahydro-2H-pyran-4- | | |
| carbonyl)octahydro-1H-cyclopenta[b]pyridine-3- | | |
| carboxamide | | |
| cis-2-(4-(cyclopentylamino)phenyl)-N-(4-methyl-3- | 110 | +++ |
| (trifluoromethyl)phenyl)-1-(oxazole-4- | | |
| carbonyl)octahydro-1H-cyclopenta[b]pyridine-3- | | |
| carboxamide | | |
| cis-2-(4-(cyclopentylamino)phenyl)-N-(4-methyl-3- | 114 | +++ |
| (trifluoromethyl)phenyl)-1-(thiazole-4- | | |
| carbonyl)octahydro-1H-cyclopenta[b]pyridine-3- | | |
| carboxamide | | |
| cis-2-(4-(cyclopentylamino)phenyl)-N-(4-methyl-3- | 115 | +++ |
| (trifluoromethyl)phenyl)-1-(pyrimidine-5- | | |
| carbonyl)octahydro-1H-cyclopenta[b]pyridine-3- | | |
| carboxamide | | |
| cis-2-(4-(cyclopentylamino)phenyl)-1-(2-fluoro-6- | 78 | +++ |
| methylbenzoyl)-N-(1-methyl-1H-pyrazolo[4,3-b]pyridin- | | |
| 6-yl)octahydro-1H-cyclopenta[b]pyridine-3-carboxamide | | |
| cis-2-(4-(cyclopentylamino)phenyl)-1-(2-fluoro-6- | 204 | +++ |
| methylbenzoyl)-5-hydroxy-N-(4-methyl-3- | | |
| (trifluoromethyl)phenyl)piperidine-3-carboxamide | | |
| cis-1-(2-fluoro-6-methylbenzoyl)-N-(4-methyl-3- | 88 | ++++ |
| (trifluoromethyl)phenyl)-2-(4-((tetrahydro-2H-pyran-4- | | |
| yl)amino)phenyl)octahydro-1H-cyclopenta[b]pyridine-3- | | |
| carboxamide | | |
| cis-2-(4-(cyclopentyl(methyl)amino)phenyl)-1-(2-fluoro- | 144 | +++ |
| 6-methylbenzoyl)-6-methyl-N-(4-methyl-3- | | |
| (trifluoromethyl)phenyl)octahydro-1H-pyrrolo[3,4- | | |
| b]pyridine-3-carboxamide | | |
| cis-2-(4-(cyclopentylamino)phenyl)-1-(2-fluoro-6- | 73 | +++ |
| methylbenzoyl)-N-(quinolin-7-yl)octahydro-1H- | | |
| cyclopenta[b]pyridine-3-carboxamide | | |
| cis-2-(4-(cyclopentylamino)phenyl)-1-(2-fluoro-6- | 59 | +++ |
| methylbenzoyl)-N-(1-(oxetan-3-yl)-1H-indazol-6- | | |
| yl)octahydro-1H-cyclopenta[b]pyridine-3-carboxamide | | |
| cis-1-(2-fluoro-6-methylbenzoyl)-N-(quinolin-7-yl)-2-(4- | 90 | +++ |
| ((tetrahydro-2H-pyran-4-yl)amino)phenyl)octahydro-1H- | | |
| cyclopenta[b]pyridine-3-carboxamide | | |
| cis-2-(4-(cyclopentylamino)phenyl)-1-(2-fluoro-6- | 121 | +++ |
| methylbenzoyl)-N-(1-methyl-1H-indazol-5- | | |
| yl)octahydrofuro[3,4-b]pyridine-3-carboxamide | | |
| (2R,3S,4aR,7aS)-2-(4-(cyclopentylamino)phenyl)-1-(2- | 126 | ++++ |
| fluoro-6-methylbenzoyl)-N-(1-methyl-1H-indazol-5- | | |
| yl)octahydrofuro[3,4-b]pyridine-3-carboxamide | | |
| cis-2-(4-(cyclopentylamino)phenyl)-1-(2-fluoro-6- | 189 | +++ |
| methylbenzoyl)-N-(1-methyl-1H-pyrazolo[4,3-b]pyridin- | | |
| 6-yl)octahydrofuro[3,4-b]pyridine-3-carboxamide | | |
| cis-1-(2-fluoro-6-methylbenzoyl)-N-(1-methyl-1H- | 158 | ++++ |
| indazol-5-yl)-2-(4-((tetrahydro-2H-pyran-4- | | |
| yl)amino)phenyl)octahydro-1H-cyclopenta[b]pyridine-3- | | |
| carboxamide. | | |
| (2R,3S,4aR,7aR)-1-(2-fluoro-6-methylbenzoyl)-N-(1- | 89 | ++++ |
| methyl-1H-indazol-5-yl)-2-(4-((tetrahydro-2H-pyran-4- | | |
| yl)amino)phenyl)octahydro-1H-cyclopenta[b]pyridine-3- | | |
| carboxamide | | |
| cis-2-(4-((3,3-dimethylmorpholino)methyl)phenyl)-1-(2- | 141 | ++ |
| fluoro-6-methylbenzoyl)-N-(4-methyl-3- | | |
| (trifluoromethyl)phenyl)octahydrofuro[3,4-b]pyridine-3- | | |
| carboxamide | | |
| (2R,3S,4aR,7aR)-2-(4-(cyclopentylamino)phenyl)-N-(4- | 51 | ++++ |
| (dimethylamino)phenyl)-1-(2-fluoro-6- | | |
| methylbenzoyl)octahydro-1H-cyclopenta[b]pyridine-3- | | |
| carboxamide | | |
| (2R,3S,4aR,7aR)-N-(4-(dimethylamino)phenyl)-1-(2- | 94 | +++ |
| fluoro-6-methylbenzoyl)-2-(4-((tetrahydro-2H-pyran-4- | | |
| yl)amino)phenyl)octahydro-1H-cyclopenta[b]pyridine-3- | | |
| carboxamide | | |
| (2R,3S,4aR,7aS)-2-(4-(cyclopentylamino)phenyl)-N-(4- | 124 | +++ |
| (dimethylamino)pheny 1)-1-(2-fluoro-6- | | |
| methylbenzoyl)octahydrofuro[3,4-b]pyridine-3- | | |
| carboxamide | | |
| (2R,3S,4aR,7aR)-1-(2-fluoro-6-methylbenzoyl)-N-(1H- | 96 | +++ |
| indazol-5-yl)-2-(4-((tetrahydro-2H-pyran-4- | | |
| yl)amino)phenyl)octahydro-1H-cyclopenta[b]pyridine-3- | | |
| carboxamide | | |
| (2R,3S,4aR,7aR)-1-(2-fluoro-6-methylbenzoyl)-N-(1- | 98 | ++++ |
| methyl-1H-indol-5-yl)-2-(4-((tetrahydro-2H-pyran-4- | | |
| yl)amino)phenyl)octahydro-1H-cyclopenta[b]pyridine-3- | | |
| carboxamide. | | |
| (2R,3S,4aR,7aS)-2-(4-(cyclopentylamino)phenyl)-1-(2- | 138 | ++ |
| fluoro-6-methylbenzoyl)-N-(1H-indazol-5- | | |
| yl)octahydrofuro[3,4-b]pyridine-3-carboxamide | | |
| (2R,3S,4aR,7aS)-2-(4-(cyclopentylamino)phenyl)-1-(2- | 139 | +++ |
| fluoro-6-methylbenzoyl)-N-(1-methyl-1H-indol-5- | | |
| yl)octahydrofuro[3,4-b]pyridine-3-carboxamide | | |
| cis-1-(2-fluoro-6-methylbenzoyl)-N-(1-methyl-1H- | 205 | ++++ |
| indazol-5-yl)-2-(4-(((R)-2-(trifluoromethyl)pyrrolidin-1- | | |
| yl)methyl)phenyl)octahydro-1H-cyclopenta[b]pyridine-3- | | |
| carboxamide | | |
| ((2R,3S)-3-(5-(tert-butyl)benzo[d]oxazol-2-yl)-2-(4- | 181 | − |
| (cyclopentylamino)phenyl)piperidin-1-yl)(2-fluoro-6- | | |
| methylphenyl)methanone | | |
| ((2R,3S)-3-(6-(tert-butyl)-1H-benzo[d]imidazol-2-yl)-2- | 180 | ++++ |
| (4-(cyclopentylamino)phenyl)piperidin-1-yl)(2-fluoro-6- | | |
| methylphenyl)methanone | | |

−: IC₅₀ > 5000 nM in migration assay; or IC₅₀ >10000 nM in Ca2+ flux assay
+: 5000 nM ≥ IC₅₀ ≥ 2000 nM in migration assay (compound shows weak activity at 2000 nM and the % inhibition is less than 50%) or 10000 nM ≥ IC₅₀ ≥ 2000 nM
++: 500 nM ≤ IC₅₀ < 2000 nM;
+++: 50 nM ≤ IC₅₀ < 500 nM;
++++: IC₅₀ < 50 nM.

### Example B3: Calcium Mobilization

U937/C5aR cells or HEK293/C5aR cells were washed by PBS and suspended in growth media at the density of 1×10⁶ cells/ml. Seed 2 cell suspension to the 384-well plate and culture for overnight. Transfer 250 nl compound solution to the cell plate using Echo, incubate for 60 min. Cells were added with Fluo-4 Direct™ dye and incubate for 50 min at 37° C. 5% CO₂ and 10 m at room temperature. Place the cell plate into FLIPRTETRA (Molecular Devices). Transfer 10 μl of 5-fold EC80 concentrations of agonist human C5a to the cell plates. Read fluorescence signal, data was calculated by GraphPad Prism and shown in Table B3.

TABLE B3

| | Com- | |
|---|---|---|
| | pound | IC₅₀ |
| Compound Name | No. | (nM) |
| (2R,3R)-2-(4-(cyclopentylamino)phenyl)-N-(4-methyl-3- | 3 | + |
| (trifluoromethyl)phenyl)-1-(pyrimidin-4-yl)piperidine-3- | | |
| carboxamide | | |
| (2R,3S)-2-(4-(cyclopentylamino)phenyl)-N-(4-methyl-3- | 4 | + |
| (trifluoromethyl)phenyl)-1-(pyrimidin-4-yl)piperidine-3- | | |
| carboxamide | | |
| (2R,3S)-2-(4-(cyclopentylamino)phenyl)-1-((2,6- | 11 | + |
| dimethylphenyl)sulfonyl)-N-(4-methyl-3- | | |
| (trifluoromethyl)phenyl)piperidine-3-carboxamide | | |
| (2R,3S)-2-(4-(cyclopentylamino)phenyl)-1-(3,5- | 190 | + |
| dimethylisoxazole-4-carbonyl)-N-(4-methyl-3- | | |
| (trifluoromethyl)phenyl)piperidine-3-carboxamide | | |
| (2R,3S)-2-(4-(cyclopentylamino)phenyl)-N-(4-methyl-3- | 5 | + |
| (trifluoromethyl)phenyl)-1-(quinazolin-4-yl)piperidine-3- | | |
| carboxamide | | |
| (2R,3S)-2-(4-(cyclopentylamino)phenyl)-N-(4-methyl-3- | 188 | +++ |
| (trifluoromethyl)phenyl)-1-(quinoline-8- | | |
| carbonyl)piperidine-3-carboxamide | | |
| (2R,3S)-1-(2-chloropyrimidin-4-yl)-2-(4- | 2 | + |
| (cyclopentylamino)phenyl)-N-(4-methyl-3- | | |
| (trifluoromethyl)phenyl)piperidine-3-carboxamide | | |
| (2R,3S)-2-(4-(cyclopentylamino)phenyl)-N-(4-methyl-3- | 161 | + |
| (trifluoromethyl)phenyl)-1-(pyrido[3,2-d]pyrimidin-4- | | |
| yl)piperidine-3-carboxamide | | |
| (2R,3R)-2-(4-(cyclopentylamino)phenyl)-N-(4-methyl-3- | 7 | + |
| (trifluoromethyl)phenyl)-1-(pyrido[3,2-d]pyrimidin-4- | | |
| yl)piperidine-3-carboxamide | | |
| (2R,3S)-2-(4-(cyclopentylamino)phenyl)-N-(4-methyl-3- | 162 | + |
| (trifluoromethyl)phenyl)-1-(pyrido[3,4-d]pyrimidin-4- | | |
| yl)piperidine-3-carboxamide | | |
| benzyl cyclopentyl(4-((2R,3S)-3-((4-methyl-3- | 166 | + |
| (trifluoromethyl)phenyl)carbamoyl)-1-(pyrido[3,2- | | |
| d]pyrimidin-4-yl)piperidin-2-yl)phenyl)carbamate | | |
| (2R,3S)-2-(4-(cyclopentylamino)phenyl)-N-(4-methyl-3- | 6 | + |
| (trifluoromethyl)phenyl)-1-(1,7-naphthyridin-8- | | |
| yl)piperidine-3-carboxamide | | |
| benzyl cyclopentyl(4-((2R,3S)-3-((4-methyl-3- | 164 | + |
| (trifluoromethyl)phenyl)carba moyl)-1-(1,7-naphthyridin- | | |
| 8-yl)piperidin-2-yl)phenyl)carbamate | | |
| benzylcyclopentyl(4-((2R,3S)-3-((4-methyl-3- | 165 | + |
| (trifluoromethyl)phenyl)carbamoyl)-1-(pyrido[3,4- | | |
| b]pyrazin-5-yl)piperidin-2-yl)phenyl)carbamate | | |
| benzyl cyclopentyl(4-((2R,3S)-3-((4-methyl-3- | 163 | + |
| (trifluoromethyl)phenyl)carbamo yl)-1-(quinazolin-4- | | |
| yl)piperidin-2-yl)phenyl)carbamate | | |
| benzyl cyclopentyl(4-((2R,3S)-3-((4-methyl-3- | 167 | + |
| (trifluoromethyl)phenyl)carba moyl)-1-(pyrido[3,4- | | |
| d]pyrimidin-4-yl)piperidin-2-yl)phenyl)carbamate | | |
| (2R,3S)-2-(4-(cyclopentylamino)phenyl)-1-((2,4- | 9 | + |
| dimethylphenyl)sulfonyl)-N-(4-methyl-3- | | |
| (trifluoromethyl)phenyl)piperidine-3-carboxamide | | |
| (2R,3S)-2-(4-(cyclopentylamino)phenyl)-1-((2,5- | 10 | − |
| dimethylphenyl)sulfonyl)-N-(4-methyl-3- | | |
| (trifluoromethyl)phenyl)piperidine-3-carboxamide | | |
| (2R,3S)-2-(4-(cyclopentylamino)phenyl)-1-((3,5- | 12 | − |
| dimethylphenyl)sulfonyl)-N-(4-methyl-3- | | |
| (trifluoromethyl)phenyl)piperidine-3-carboxamide | | |
| (2R,3S)-2-(4-(cyclopentylamino)phenyl)-1- | 13 | + |
| (mesitylsulfonyl)-N-(4-methyl-3- | | |
| (trifluoromethyl)phenyl)piperidine-3-carboxamide | | |
| (2R,3S)-2-(4-(cyclopentylamino)phenyl)-1-((4-fluoro-2- | 15 | + |
| methylphenyl)sulfonyl)-N-(4-methyl-3- | | |
| (trifluoromethyl)phenyl)piperidine-3-carboxamide | | |
| (2R,3S)-1-((3-chloro-2-methylphenyl)sulfonyl)-2-(4- | 16 | − |
| (cyclopentylamino)phenyl)-N-(4-methyl-3- | | |
| (trifluoromethyl)phenyl)piperidine-3-carboxamide | | |
| (2R,3S)-2-(4-(cyclopentylamino)phenyl)-1-((5-fluoro-2- | 17 | + |
| methylphenyl)sulfonyl)-N-(4-methyl-3- | | |
| (trifluoromethyl)phenyl)piperidine-3-carboxamide | | |
| (2R,3S)-1-((3-fluoro-2-methylphenyl)sulfonyl)-2-(4- | 18 | − |
| (cyclopentylamino)phenyl)-N-(4-methyl-3- | | |
| (trifluoromethyl)phenyl)piperidine-3-carboxamide | | |
| (2R,3S)-2-(4-(cyclopentylamino)phenyl)-1-((2,6- | 19 | + |
| difluorophenyl)sulfonyl)-N-(4-methyl-3- | | |
| (trifluoromethyl)phenyl)piperidine-3-carboxamide | | |
| (2R,3S)-2-(4-(cyclopentylamino)phenyl)-1-((2,6- | 20 | − |
| dichlorophenyl)sulfonyl)-N-(4-methyl-3- | | |
| (trifluoromethyl)phenyl)piperidine-3-carboxamide | | |
| methyl 2-(((2R,3S)-2-(4-(cyclopentylamino)phenyl)-3- | 21 | − |
| ((4-methyl-3- | | |
| (trifluoromethyl)phenyl)carbamoyl)piperidin-1- | | |
| yl)sulfonyl)-3-methylbenzoate | | |
| (2R,3S)-2-(4-(cyclopentylamino)phenyl)-N-(4-methyl-3- | 22 | − |
| (trifleoromethyl)phenyl)-1-(o-tolylsulfonyl)piperidine-3- | | |
| carboxamide | | |
| (2R,3S)-2-(4-(cyclopentylamino)phenyl)-1-((2- | 23 | + |
| methoxyphenyl)sulfonyl)-N-(4-methyl-3- | | |
| (trifluoromethyl)phenyl)piperidine-3-carboxamide | | |
| (2R,3S)-2-(4-(cyclopentylamino)phenyl)-N-(4-methyl-3- | 24 | − |
| (trifluoromethyl)phenyl)-1-((2- | | |
| (trifluoromethoxy)phenyl)sulfonyl)piperidine-3- | | |
| carboxamide | | |
| (2R,3S)-2-(4-(cyclopentylamino)phenyl)-1-((2- | 25 | − |
| fluorophenyl)sulfonyl)-N-(4-methyl-3- | | |
| (trifluoromethyl)phenyl)piperidine-3-carboxamide | | |
| (2R,3S)-1-((2-chlorophenyl)sulfonyl)-2-(4- | 26 | − |
| (cyclopentylamino)phenyl)-N-(4-methyl-3- | | |
| (trifluoromethyl)phenyl)piperidine-3-carboxamide | | |
| (2R,3S)-1-((2-bromophenyl)sulfonyl)-2-(4- | 27 | + |
| (cyclopentylamino)phenyl)-N-(4-methyl-3- | | |
| (trifluoromethyl)phenyl)piperidine-3-carboxamide | | |
| (2R,3S)-2-(4-(cyclopentylamino)phenyl)-N-(4-methyl-3- | 28 | + |
| (trifluoromethyl)phenyl)-1-((2- | | |
| (trifluoromethyl)phenyl)sulfonyl)piperidine-3- | | |
| carboxamide | | |
| (2R,3S)-2-(4-(cyclopentylamino)phenyl)-N-(4-methyl-3- | 29 | + |
| (trifluoromethyl)phenyl)-1-((2- | | |
| (methylsulfonyl)phenyl)sulfonyl)piperidine-3- | | |
| carboxamide | | |
| (2R,3S)-1-((2-cyanophenyl)sulfonyl)-2-(4- | 30 | + |
| (cyclopentylamino)phenyl)-N-(4-methyl-3- | | |
| (trifluoromethyl)phenyl)piperidine-3-carboxamide | | |
| (2R,3S)-2-(4-(cyclopentylamino)phenyl)-N-(4-methyl-3- | 14 | + |
| (trifluoromethyl)phenyl)-1-((2- | | |
| nitrophenyl)sulfonyl)piperidine-3-carboxamide | | |
| Methyl 2-(((2R,3S)-2-(4-(cyclopentylamino)phenyl)-3- | 31 | + |
| ((4-methyl-3- | | |
| (trifluoromethyl)phenyl)carbamoyl)piperidin-1- | | |
| yl)sulfonyl)benzoate | | |
| (2R,3S)-2-(4-(cyclopentylamino)phenyl)-N-(4-methyl-3- | 32 | + |
| (trifluoromethyl)phenyl)-1-(naphthalen-2- | | |
| ylsulfonyl)piperidine-3-carboxamide | | |
| (2R,3S)-2-(4-(cyclopentylamino)phenyl)-N-(4-methyl-3- | 33 | + |
| (trifluoromethyl)phenyl)-1-(naphthalen-1- | | |
| ylsulfonyl)piperidine-3-carboxamide | | |
| (2R,3S)-2-(4-(cyclopentylamino)phenyl)-N-(4-methyl-3- | 34 | − |
| (trifluoromethyl)phenyl)-1-(phenylsulfonyl)piperidine-3- | | |
| carboxamide | | |
| (2R,3S)-2-(4-(cyclopentylamino)phenyl)-N-(4-methyl-3- | 35 | − |
| (trifluoromethyl)phenyl)-1-(pyridin-3- | | |
| ylsulfonyl)piperidine-3-carboxamide | | |
| (2R,3S)-1-((2-chloropyridin-3-yl)sulfonyl)-2-(4- | 36 | + |
| (cyclopentylamino)phenyl)-N-(4-methyl-3- | | |
| (trifluoromethyl)phenyl)piperidine-3-carboxamide | | |
| (2R,3S)-2-(4-(cyclopentylamino)phenyl)-N-(4-methyl-3- | 37 | + |
| (trifluoromethyl)phenyl)-1- | | |
| ((perfluorophenyl)sulfonyl)piperidine-3-carboxamide | | |
| (2R,3S)-2-(4-(cyclopentylamino)phenyl)-N-(4-methyl-3- | 38 | − |
| (trifluoromethyl)phenyl)-1-((1,3,5-trimethyl-1H-pyrazol- | | |
| 4-yl)sulfonyl)piperidine-3-carboxamide | | |
| (2R,3S)-2-(4-(cyclopentylamino)phenyl)-1-((3,5- | 8 | + |
| dimethylisoxazol-4-yl)sulfonyl)-N-(4-methyl-3- | | |
| (trifluoromethyl)phenyl)piperidine-3-carboxamide | | |
| (2R,3S)-1-(benzylsulfonyl)-2-(4- | 40 | + |
| (cyclopentylamino)phenyl)-N-(4-methyl-3-(tri | | |
| fluoromethyl)phenyl)piperidine-3-carboxamide | | |
| (2R,3S)-2-(4-(cyclopentyl(1,7-naphthyridin-8- | 168 | + |
| yl)amino)phenyl)-N-(4-methyl-3- | | |
| (trifluoromethyl)phenyl)piperidine-3-carboxamide | | |
| (2R,3R)-2-(4-(cyclopentyl(1,7-naphthyridin-8- | 171 | + |
| yl)amino)phenyl)-N-(4-methyl-3- | | |
| (trifluoromethyl)phenyl)piperidine-3-carboxamide | | |
| (2R,3S)-2-(4-(cyclopentyl(thieno[2,3-c]pyridin-7- | 173 | + |
| yl)amino)phenyl)-N-(4-methyl-3- | | |
| (trifluoromethyl)phenyl)piperidine-3-carboxamide | | |
| (2R,3S)-2-(4-(cyclopentyl(isoquinolin-1- | 175 | + |
| yl)amino)phenyl)-N-(4-methyl-3- | | |
| (trifluoromethyl)phenyl)piperidine-3-carboxamide | | |
| (2R,3S)-2-(4-(cyclopentyl(quinazolin-4- | 177 | + |
| yl)amino)phenyl)-N-(4-methyl-3- | | |
| (trifluoromethyl)phenyl)piperidine-3-carboxamide | | |
| (2R,3S)-2-(4-(cyclopentyl(phthalazin-1- | 179 | + |
| yl)amino)phenyl)-N-(4-methyl-3- | | |
| (trifluoromethyl)phenyl)piperidine-3-carboxamide | | |
| (2R,3S)-2-(4-(cyclopentyl(thiazolo[4,5-c]pyridin-4- | 169 | + |
| yl)amino)phenyl)-N-(4-methyl-3- | | |
| (trifluoromethyl)phenyl)piperidine-3-carboxamide | | |
| (2R,3S)-2-(4-(cyclopentyl(pyrido[3,4-b]pyrazin-5- | 172 | + |
| yl)amino)phenyl)-N-(4-methyl-3- | | |
| (trifluoromethyl)phenyl)piperidine-3-carboxamide | | |
| (2R,3S)-2-(4-(cyclopentyl(pyrido[3,2-d]pyrimidin-4- | 176 | + |
| yl)amino)phenyl)-N-(4-methyl-3- | | |
| (trifluoromethyl)phenyl)piperidine-3-carboxamide | | |
| (2R,3R)-2-(4-(cyclopentyl(pyrido[3,2-d]pyrimidin-4- | 174 | + |
| yl)amino)phenyl)-N-(4-methyl-3- | | |
| (trifluoromethyl)phenyl)piperidine-3-carboxamide | | |
| (2R,3S)-2-(4-(cyclopentyl(pyrido[3,4-d]pyrimidin-4- | 178 | + |
| yl)amino)phenyl)-N-(4-methyl-3- | | |
| (trifluoromethyl)phenyl)piperidine-3-carboxamide | | |
| cis-1-(2-fluoro-6-methylbenzoyl)-N-(4-methyl-3- | 203 | + |
| (trifluoromethyl)phenyl)-2-(2-oxaspiro[4.5]decan-8- | | |
| yl)piperidine-3-carboxamide | | |
| (2R,3S)-2-(4-(N-cyclopentyl-2-fluoro-6- | 188 | + |
| methylbenzamido)phenyl)-N-(4-methyl-3- | | |
| (trifluoromethyl)phenyl)piperidine-3-carboxamide | | |
| cis-3-(4-aminophenyl)-4-(2-fluoro-6-methylbenzoyl)-N- | 187 | + |
| (4-methyl-3-(trifluoro methyl)phenyl)morpholine-2- | | |
| carboxamide | | |
| cis-4-(4-(cyclopentylamino)phenyl)-N-(4-methyl-3- | 43 | + |
| (trifluoromethyl)phenyl)-6-oxo-2,3,4,6,11,11a- | | |
| hexahydro-1H-pyrido[1,2-b]isoquinoline-3-carboxamide | | |
| (3S,4R)-4-(4-(cyclopentylamino)phenyl)-N-(4-methyl-3- | 44 | ++ |
| (trifluoromethyl)phenyl)-6-oxo-1,2,3,4,6,11,12,12a- | | |
| octahydrobenzo[e]pyrido[1,2-a]azepine-3-carboxamide | | |
| (3R,4S)-4-(4-(cyclopentylamino)phenyl)-N-(4-methyl-3- | 45 | + |
| (trifluoromethyl)phenyl)-6-oxo-1,2,3,4,6,11,12,12a- | | |
| octahydrobenzo[e]pyrido[1,2-a]azepine-3-carboxamide | | |
| cis-2-(4-(cyclopentylamino)phenyl)-1-(2-fluoro-6- | 193 | ++++ |
| methylbenzoyl)-N-(4-methyl-3- | | |
| (trifluoromethyl)phenyl)octahydro-1H- | | |
| cyclopenta[b]pyridine-3-carboxamide | | |
| (2R,3S,4aR,7aR)-2-(4-(cyclopentylamino)phenyl)-1-(2- | 47 | ++++ |
| fluoro-6-methylbenzoyl)-N-(4-methyl-3- | | |
| (trifluoromethyl)phenyl)octahydro-1H- | | |
| cyclopenta[b]pyridine-3-carboxamide | | |
| (2S,3R,4aS,7aS)-2-(4-(cyclopentylamino)phenyl)-1-(2- | 48 | + |
| fluoro-6-methylbenzoyl)-N-(4-methyl-3- | | |
| (trifluoromethyl)phenyl)octahydro-1H- | | |
| cyclopenta[b]pyridine-3-carboxamide | | |
| (2R,3S)-2-(4-(cyclopentylamino)phenyl)-N-(4-methyl-3- | 200 | + |
| (trifluoromethyl)phenyl)piperidine-3-carboxamide | | |
| cis-2-(4-(cyclopentylamino)phenyl)-1-(2-fluoro-6- | 118 | +++ |
| methylbenzoyl)-N-(4-methyl-3- | | |
| (trifluoromethyl)phenyl)octahydrofuro[3,4-b]pyridine-3- | | |
| carboxamide | | |
| (2R,3S,4aR,7aS)-2-(4-(cyclopentylamino)phenyl)-1-(2- | 119 | +++ |
| fluoro-6-methylbenzoy 1)-N-(4-methyl-3- | | |
| (trifluoromethyl)phenyl)octahydrofuro[3,4-b]pyridine-3- | | |
| carboxamide | | |
| cis-2-(4-(cyclopentylamino)phenyl)-1-(2-fluoro-6- | 206 | + |
| methylbenzoyl)-N-(4-methyl-3-(trifluoromethyl)phenyl)- | | |
| 6-(2,2,2-trifluoroethyl)octahydro-1H-pyrrolo[3,4- | | |
| b]pyridine-3-carboxamide | | |
| cis-4-(4-(cyclopentylamino)phenyl)-7-fluoro-N-(4- | 41 | + |
| methyl-3-(trifluorometh yl)phenyl)-6-oxo- | | |
| 1,2,3,4,6,11,12,12a-octahydrobenzo[e]pyrido[1,2- | | |
| a]azepine-3-carboxamide | | |
| (2R,3S,4aR,7aR)-2-(4-(cyclopentylamino)phenyl)-1-(2- | 52 | + |
| fluoro-6-methylbenzoyl)-N-(tetrahydro-2H-pyran-4- | | |
| yl)octahydro-1H-cyclopenta[b]pyridine-3-carboxamide | | |
| (2R,3S,4aR,7aR)-2-(4-(cyclopentylamino)phenyl)-1-(2- | 53 | + |
| fluoro-6-methylbenzoyl)-N-(1-methylpiperidin-4- | | |
| yl)octahydro-1H-cyclopenta[b]pyridine-3-carboxamide | | |
| (2R,3S,4aR,7aR)-2-(4-(cyclopentylamino)phenyl)-1-(2- | 54 | + |
| fluoro-6-methylbenzoyl)-N-(1-methyl-1H-pyrazol-4- | | |
| yl)octahydro-1H-cyclopenta[b]pyridine-3-carboxamide | | |
| cis-2-(4-(cyclopentylamino)phenyl)-1-(2-fluoro-6- | 79 | + |
| methylbenzoyl)-N-(2-(trifluor omethyl)pyridin-4- | | |
| yl)octahydro-1H-cyclopenta[b]pyridine-3-carboxamide | | |
| cis-2-(4-(cyclopentylamino)phenyl)-1-(2-fluoro-6- | 77 | + |
| methylbenzoyl)-N-(2-methyl-1,2,3,4- | | |
| tetrahydroisoquinolin-6-yl)octahydro-1H- | | |
| cyclopenta[b]pyridine-3-carboxamide | | |
| (2R,3S,4aR,7aR)-2-(4-(cyclopentylamino)phenyl)-1-(2- | 185 | + |
| fluoro-6-methylbenzoyl)-N-(4-methyl-3- | | |
| (trifluoromethyl)benzyl)octahydro-1H- | | |
| cyclopenta[b]pyridine-3-carboxamide | | |
| cis-2-(4-(cyclopentylamino)phenyl)-1-(2-fluoro-6- | 56 | +++ |
| methylbenzoyl)-N-(3-fluoroph enyl)octahydro-1H- | | |
| cyclopenta[b]pyridine-3-carboxamide | | |
| cis-2-(4-(cyclopentylamino)phenyl)-1-(2-fluoro-6- | 57 | + |
| methylbenzoyl)-N-(pyridin-3-yl)octahydro-1H- | | |
| cyclopenta[b]pyridine-3-carboxamide | | |
| cis-2-(4-(cyclopentylamino)phenyl)-1-(2-fluoro-6- | 58 | +++ |
| methylbenzoyl)-N-(2-methylpyrimidin-5-yl)octahydro- | | |
| 1H-cyclopenta[b]pyridine-3-carboxamide | | |
| cis-N-(4-chloro-3-(trifluoromethyl)phenyl)-2-(4- | 60 | +++ |
| (cyclopentylamino)phenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-N-(4-fluoro-3- | 61 | + |
| (trifluoromethyl)phenyl)-1-(2-fluoro-6- | | |
| methylbenzoyl)octahydro-1H-cyclopenta[b]pyridine-3- | | |
| carboxamide | | |
| cis-N-(3-cyano-4-methylphenyl)-2-(4- | 62 | +++ |
| (cyclopentylamino)phenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-1-(2-fluoro-6- | 63 | ++ |
| methylbenzoyl)-N-(6-methylpyridin-3-yl)octahydro-1H- | | |
| cyclopenta[b]pyridine-3-carboxamide | | |
| cis-2-(4-(cyclopentylamino)phenyl)-N-(3,4- | 64 | + |
| dichlorophenyl)-1-(2-fluoro-6-methylbenzoyl)octahydro- | | |
| 1H-cyclopenta[b]pyridine-3-carboxamide | | |
| cis-2-(4-(cyclopentylamino)phenyl)-N-(3,4- | 65 | +++ |
| difluorophenyl)-1-(2-fluoro-6-methylbenzoyl)octahydro- | | |
| 1H-cyclopenta[b]pyridine-3-carboxamide | | |
| cis-2-(4-(cyclopentylamino)phenyl)-1-(2-fluoro-6- | 147 | +++ |
| methylbenzoyl)-N-(1-methyl-1H-indazol-5-yl)octahydro- | | |
| 1H-cyclopenta[b]pyridine-3-carboxamide | | |
| (2R,3S,4aR,7aR)-2-(4-(cyclopentylamino)phenyl)-1-(2- | 49 | ++ |
| fluoro-6-methylbenzoyl)-N-(1-methyl-1H-indazol-5- | | |
| yl)octahydro-1H-cyclopenta[b]pyridine-3-carboxamide | | |
| cis-2-(4-(cyclopentylamino)phenyl)-1-(2-fluoro-6- | 66 | +++ |
| methylbenzoyl)-N-(1-methyl-1H-indazol-6-yl)octahydro- | | |
| 1H-cyclopenta[b]pyridine-3-carboxamide | | |
| (2R,3S,4aR,7aR)-2-(4-(cyclopentylamino)phenyl)-1-(2- | 50 | ++ |
| fluoro-6-methylbenzoyl)-N-(1-methyl-1H-indazol-6- | | |
| yl)octahydro-1H-cyclopenta[b]pyridine-3-carboxamide | | |
| cis-N-(benzo[d]oxazol-6-yl)-2-(4- | 67 | +++ |
| (cyclopentylamino)phenyl)-1-(2-fluoro-6- | | |
| methylbenzoyl)octahydro-1H-cyclopenta[b]pyridine-3- | | |
| carboxamide | | |
| cis-N-(benzo[d]thiazol-6-yl)-2-(4- | 69 | + |
| (cyclopentylamino)phenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-N-(2,3- | 71 | + |
| dihydrobenzo[b][1,4]dioxin-6-yl)-1-(2-fluoro-6- | | |
| methylbenzoyl)octahydro-1H-cyclopenta[b]pyridine-3- | | |
| carboxamide | | |
| cis-3-(6-chloro-1,2,3,4-tetrahydroisoquinoline-2- | 72 | + |
| carbonyl)-2-(4-(cyclopentyl amino)phenyl)octahydro-1H- | | |
| cyclopenta[b]pyridin-1-yl)(2-fluoro-6- | | |
| methylphenyl)methanone | | |
| cis-2-(4-(cyclopentylamino)phenyl)-1-(2-fluoro-6- | 46 | +++ |
| methylbenzoyl)-N-(3-(trifluoromethyl)phenyl)octahydro- | | |
| 1H-cyclopenta[b]pyridine-3-carboxamide | | |
| cis-N-(3-chlorophenyl)-2-(4-(cyclopentylamino)phenyl)- | 55 | ++ |
| 1-(2-fluoro-6-methylbenzoyl)octahydro-1H- | | |
| cyclopenta[b]pyridine-3-carboxamide | | |
| cis-2-(4-(cyclopentyl(methyl)amino)phenyl)-1-(2-fluoro- | 201 | + |
| 6-methylbenzoyl)-N-(2-methyl-1,2,3,4- | | |
| tetrahydroisoquinolin-6-yl)octahydro-1H- | | |
| cyclopenta[b]pyridine-3-carboxamide | | |
| tert-butyl 6-cis-2-(4-(cyclopentylamino)phenyl)-1-(2- | 202 | + |
| fluoro-6-methylbenzoyl)octahydro-1H- | | |
| cyclopenta[b]pyridine-3-carboxamido)-3,4- | | |
| dihydroisoquinoline-2(1H)-carboxylate | | |
| cis-2-(4-(cyclopentylamino)phenyl)-1-(2-fluoro-6- | 184 | + |
| methylbenzoyl)-N-(1,2,3,4-tetrahydroisoquinolin-6- | | |
| yl)octahydro-1H-cyclopenta[b]pyridine-3-carboxamide | | |
| cis-2-(4-(cyclopentylamino)phenyl)-N-(3- | 76 | + |
| (dimethylphosphoryl)-4-methylphenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-1-(2-fluoro-6- | 70 | + |
| methylbenzoyl)-N-(3-(methyls ulfonyl)phenyl)octahydro- | | |
| 1H-cyclopenta[b]pyridine-3-carboxamide | | |
| cis-2-(4-(cyclopentylamino)phenyl)-N-(4-methyl-3- | 111 | + |
| (trifluoromethyl)phenyl)-1-(tetrahydro-2H-pyran-4- | | |
| carbonyl)octahydro-1H-cyclopenta[b]pyridine-3- | | |
| carboxamide | | |
| cis-2-(4-(cyclopentylamino)phenyl)-1-(1-methyl-1H- | 112 | − |
| pyrazole-4-carbonyl)-N-(4-methyl-3- | | |
| (trifluoromethyl)phenyl)octahydro-1H- | | |
| cyclopenta[b]pyridine-3-carboxamide | | |
| cis-2-(4-(cyclopentylamino)phenyl)-N-(4-methyl-3- | 110 | − |
| (trifluoromethyl)phenyl)-1-(oxazole-4- | | |
| carbonyl)octahydro-1H-cyclopenta[b]pyridine-3- | | |
| carboxamide | | |
| cis-2-(4-(cyclopentylamino)phenyl)-1-(1-methyl-1H- | 113 | − |
| imidazole-4-carbonyl)-N-(4-methyl-3- | | |
| (trifluoromethyl)phenyl)octahydro-1H- | | |
| cyclopenta[b]pyridine-3-carboxamide | | |
| cis-2-(4-(cyclopentylamino)phenyl)-N-(4-methyl-3- | 114 | − |
| (trifluoromethyl)phenyl)-1-(thiazole-4- | | |
| carbonyl)octahydro-1H-cyclopenta[b]pyridine-3- | | |
| carboxamide | | |
| cis-2-(4-(cyclopentylamino)phenyl)-N-(4-methyl-3- | 115 | − |
| (trifluoromethyl)phenyl)-1-(pyrimidine-5- | | |
| carbonyl)octahydro-1H-cyclopenta[b]pyridine-3- | | |
| carboxamide | | |
| cis-2-(4-(cyclopentylamino)phenyl)-1-(2-fluoro-6- | 68 | ++ |
| methylbenzoyl)-N-(4-formami do-3- | | |
| hydroxyphenyl)octahydro-1H-cyclopenta[b]pyridine-3- | | |
| carboxamide | | |
| cis-2-(4-(cyclopentylamino)phenyl)-1-(2-fluoro-6- | 78 | ++ |
| methylbenzoyl)-N-(1-methyl-1H-pyrazolo[4,3-b]pyridin- | | |
| 6-yl)octahydro-1H-cyclopenta[b]pyridine-3-carboxamide | | |
| cis-2-(4-(cyclopentylamino)phenyl)-1-(2-fluoro-6- | 204 | + |
| methylbenzoyl)-5-hydroxy-N-(4-methyl-3- | | |
| (trifluoromethyl)phenyl)piperidine-3-carboxamide | | |
| cis-1-(2-fluoro-6-methylbenzoyl)-N-(4-methyl-3- | 88 | ++++ |
| (trifluoromethyl)phenyl)-2-(4-((tetrahydro-2H-pyran-4- | | |
| yl)amino)phenyl)octahydro-1H-cyclopenta[b]pyridine-3- | | |
| carboxamide | | |
| cis-N-(5-chloro-6-(2H-1,2,3-triazol-2-yl)pyridin-3-yl)-2- | 75 | − |
| (4-(cyclopentylamino)phenyl)-1-(2-fluoro-6- | | |
| methylbenzoyl)octahydro-1H-cyclopenta[b]pyridine-3- | | |
| carboxamide | | |
| cis-2-(4-(cyclopentylamino)phenyl)-1-(2-fluoro-6- | 74 | − |
| methylbenzoyl)-N-(1-methyl-1H-benzo[d]imidazol-6- | | |
| yl)octahydro-1H-cyclopenta[b]pyridine-3-carboxamide | | |
| cis-2-(4-(cyclopentyl(methyl)amino)phenyl)-1-(2-fluoro- | 144 | + |
| 6-methylbenzoyl)-6-methyl-N-(4-methyl-3- | | |
| (trifluoromethyl)phenyl)octahydro-1H-pyrrolo[3,4- | | |
| b]pyridine-3-carboxamide | | |
| cis-2-(4-(cyclopentylamino)phenyl)-1-(2-fluoro-6- | 73 | +++ |
| methylbenzoyl)-N-(quinolin-7-yl)octahydro-1H- | | |
| cyclopenta[b]pyridine-3-carboxamide | | |
| cis-2-(4-(cyclopentylamino)phenyl)-1-(2-fluoro-6- | 59 | ++ |
| methylbenzoyl)-N-(1-(oxetan-3-yl)-1H-indazol-6- | | |
| yl)octahydro-1H-cyclopenta[b]pyridine-3-carboxamide | | |
| cis-1-(2-fluoro-6-methylbenzoyl)-N-(quinolin-7-yl)-2-(4- | 90 | +++ |
| ((tetrahydro-2H-pyran-4-yl)amino)phenyl)octahydro-1H- | | |
| cyclopenta[b]pyridine-3-carboxamide | | |
| cis-2-(4-(cyclopentylamino)phenyl)-1-(2-fluoro-6- | 121 | +++ |
| methylbenzoyl)-N-(1-methyl-1H-indazol-5- | | |
| yl)octahydrofuro[3,4-b]pyridine-3-carboxamide | | |
| (2R,3S,4aR,7aS)-2-(4-(cyclopentylamino)phenyl)-1-(2- | 126 | + |
| fluoro-6-methylbenzoyl)-N-(1-methyl-1H-indazol-5- | | |
| yl)octahydrofuro[3,4-b]pyridine-3-carboxamide | | |
| cis-2-(4-(cyclopentylamino)phenyl)-1-(2-fluoro-6- | 189 | + |
| methylbenzoyl)-N-(1-methyl-1H-pyrazolo[4,3-b]pyridin- | | |
| 6-yl)octahydrofuro[3,4-b]pyridine-3-carboxamide | | |
| cis-1-(2-fluoro-6-methylbenzoyl)-N-(1-methyl-1H- | 158 | +++ |
| indazol-5-yl)-2-(4-((tetrahydro-2H-pyran-4- | | |
| yl)amino)phenyl)octahydro-1H-cyclopenta[b]pyridine-3- | | |
| carboxamide | | |
| (2R,3S,4aR,7aR)-1-(2-fluoro-6-methylbenzoyl)-N-(1- | 89 | ++ |
| methyl-1H-indazol-5-yl)-2-(4-((tetrahydro-2H-pyran-4- | | |
| yl)amino)phenyl)octahydro-1H-cyclopenta[b]pyridine-3- | | |
| carboxamide | | |
| cis-1-(2-fluoro-6-methylbenzoyl)-N-(4-methyl-3- | 135 | ++ |
| (trifluoromethyl)phenyl)-2-(4-((tetrahydro-2H-pyran-4- | | |
| yl)amino)phenyl)octahydrofuro[3,4-b]pyridine-3- | | |
| carboxamide | | |
| cis-N-(4-(dimethylamino)phenyl)-1-(2-fluoro-6- | 136 | + |
| methylbenzoyl)-2-(4-((tetrahydro-2H-pyran-4- | | |
| yl)amino)phenyl)octahydrofuro[3,4-b]pyridine-3- | | |
| carboxamide | | |
| cis-2-(4-((3,3-dimethylmorpholino)methyl)phenyl)-1-(2- | 141 | + |
| fluoro-6-methylbenzoyl)-N-(4-methyl-3- | | |
| (trifluoromethyl)phenyl)octahydrofuro[3,4-b]pyridine-3- | | |
| carboxamide | | |
| (2R,3S,4aR,7aR)-2-(4-(cyclopentylamino)phenyl)-N-(4- | 51 | ++ |
| (dimethylamino)phenyl)-1-(2-fluoro-6- | | |
| methylbenzoyl)octahydro-1H-cyclopenta[b]pyridine-3- | | |
| carboxamide | | |
| (2R,3S,4aR,7aR)-N-(4-(dimethylamino)phenyl)-1-(2- | 94 | + |
| fluoro-6-methylbenzoyl)-2-(4-((tetrahydro-2H-pyran-4- | | |
| yl)amino)phenyl)octahydro-1H-cyclopenta[b]pyridine-3- | | |
| carboxamide. | | |
| cis-1-(2-fluoro-6-methylbenzoyl)-N-(1-methyl-1H- | 117 | + |
| indazol-5-yl)-2-(4-((1-methylpiperidin-4- | | |
| yl)amino)phenyl)octahydro-1H-cyclopenta[b]pyridine-3- | | |
| carboxamide | | |
| cis-1-(2-fluoro-6-methylbenzoyl)-N-phenyl-2-(4- | 129 | + |
| ((tetrahydro-2H-pyran-4-yl) | | |
| amino)phenyl)octahydrofuro[3,4-b]pyridine-3- | | |
| carboxamide | | |
| cis-1-(2-fluoro-6-methylbenzoyl)-N-(pyridin-3-yl)-2-(4- | 128 | + |
| ((tetrahydro-2H-pyran-4- | | |
| yl)amino)phenyl)octahydrofuro[3,4-b]pyridine-3- | | |
| carboxamide | | |
| cis-1-(2-fluoro-6-methylbenzoyl)-N-(1-methyl-1H- | 127 | + |
| pyrazol-4-yl)-2-(4-((tetrahydro-2H-pyran-4- | | |
| yl)amino)phenyl)octahydrofuro[3,4-b]pyridine-3- | | |
| carboxamide | | |
| (2R,3S,4aR,7aS)-2-(4-(cyclopentylamino)phenyl)-N-(4- | 124 | + |
| (dimethylamino)pheny l)-1-(2-fluoro-6- | | |
| methylbenzoyl)octahydrofuro[3,4-b]pyridine-3- | | |
| carboxamide | | |
| (2R,3S,4aR,7aR)-1-(2-fluoro-6-methylbenzoyl)-N-(1H- | 96 | + |
| indazol-5-yl)-2-(4-((tetrahydro-2H-pyran-4- | | |
| yl)amino)phenyl)octahydro-1H-cyclopenta[b]pyridine-3- | | |
| carboxamide | | |
| (2R,3S,4aR,7aR)-1-(2-fluoro-6-methylbenzoyl)-N-(1- | 98 | ++ |
| methyl-1H-indol-5-yl)-2-(4-((tetrahydro-2H-pyran-4- | | |
| yl)amino)phenyl)octahydro-1H-cyclopenta[b]pyridine-3- | | |
| carboxamide | | |
| (2R,3S,4aR,7aS)-2-(4-(cyclopentylamino)phenyl)-1-(2- | 138 | + |
| fluoro-6-methylbenzoyl)-N-(1H-indazol-5- | | |
| yl)octahydrofuro[3,4-b]pyridine-3-carboxamide | | |
| (2R,3S,4aR,7aS)-2-(4-(cyclopentylamino)phenyl)-1-(2- | 139 | + |
| fluoro-6-methylbenzoyl)-N-(1-methyl-1H-indol-5- | | |
| yl)octahydrofuro[3,4-b]pyridine-3-carboxamide | | |
| ((2R,3S)-3-(5-(tert-butyl)benzo[d]oxazol-2-yl)-2-(4- | 181 | + |
| (cyclopentylamino)phenyl)piperidin-1-yl)(2-fluoro-6- | | |
| methylphenyl)methanone | | |
| ((2R,3S)-3-(6-(tert-butyl)-1H-benzo[d]imidazol-2-yl)-2- | 180 | + |
| (4-(cyclopentylamino)phenyl)piperidin-1-yl)(2-fluoro-6- | | |
| methylphenyl)methanone | | |
| (2R,3S)-2-(4-(cyclopentylamino)phenyl)-1-(2-fluoro-6- | 159 | + |
| methylbenzyl)-N-(4-methyl-3- | | |
| (trifluoromethyl)phenyl)piperidine-3-carboxamide | | |
| ((2R,3R)-2-(4-(cyclopentylamino)phenyl)-3-(((4-methyl- | 160 | + |
| 3-(trifluoromethyl)phenyl)amino)methyl)piperidin-1- | | |
| yl)(2-fluoro-6-methylphenyl)methanone | | |

−: IC₅₀ > 5000 nM in migration assay; or IC₅₀ > 10000 nM in Ca2+ flux assay
+: 5000 nM ≥ IC₅₀ > 2000 nM in migration assay (compound shows weak activity at 2000 nM and the % inhibition is less than 50%) or 10000 nM ≥ IC₅₀ ≥ 2000 nM
++: 500 nM ≤ IC₅₀ < 2000 nM;
+++: 50 nM ≤ IC₅₀ < 500 nM;
++++: IC₅₀ < 50 nM.

### Example B4: In Vivo Characterization of Selected Compounds

Compound Nos. 47, 49, and 89 were used for in vivo characterization of their activities. The names of the compounds are shown in Table B4.

TABLE B4

| Compound No. | Compound name |
|---|---|
| 47 | (2R,3S,4aR,7aR)-2-(4-(cyclopentylamino)phenyl)-1-(2- |
| | fluoro-6-methylbenzoyl)-N-(4-methyl-3- |
| | (trifluoromethyl)phenyl)octahydro-1H- |
| | cyclopenta[b]pyridine-3-carboxamide |
| 49 | (2R,3S,4aR,7aR)-2-(4-(cyclopentylamino)phenyl)-1-(2- |
| | fluoro-6-methylbenzoyl)-N-(1-methyl-1H-indazol-5- |
| | yl)octahydro-1H-cyclopenta[b]pyridine-3-carboxamide |
| 89 | (2R,3S,4aR,7aR)-1-(2-fluoro-6-methylbenzoyl)-N-(1- |
| | methyl-1H-indazol-5-yl)-2-(4-((tetrahydro-2H-pyran-4- |
| | yl)amino)phenyl)octahydro-1H-cyclopenta[b]pyridine-3- |
| | carboxamide |

### C5a Induced Neutropenia in a Cynomolgus Model

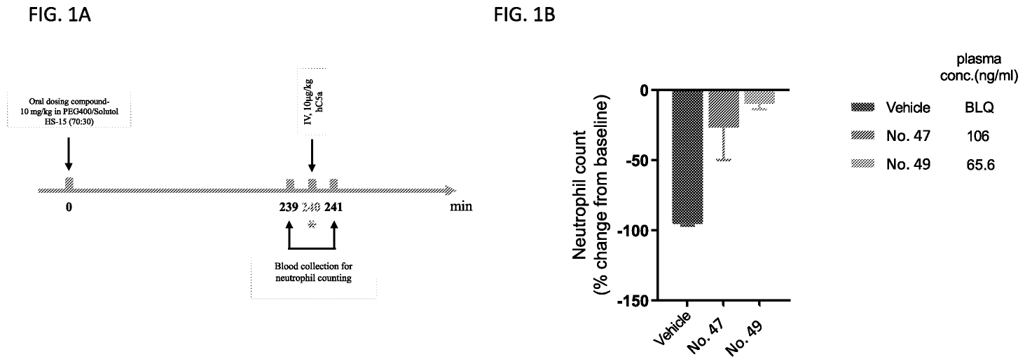

To study the efficacy of compounds in a non-human primate model, human C5a (hC5a) induced neutropenia is studied in a cynomolgus model. Intravenous injection of hC5a induced upregulation of adhesion molecules on blood vessel walls, leading to decreased neutrophils in the blood stream and attached to the vascular walls. Monkeys were pre-dosed with vehicle or specific compound, and 4 hours later, hC5a (10 g/kg, ACROBiosystems) was administrated and 1 minute later neutrophils are quantified in peripheral blood. The experimental design is shown in FIG. 1A.

FIG. 1B shows that compound Nos. 47 and 49 effectively rescued the neutropenia induced by human C5a in cynomolgus monkey compared to vehicle as the negative control. The percent change in the number of neutrophils in the blood collected after C5a injection (241 min) was calculated relative to the sample collected prior to C5a injection (239 min). The plasma concentration of each compound was calculated as the average concentration prior to C5a injection of 2 individual monkeys.

### C5a Induced Neutropenia in Human C5aR Knock-In Mice Model

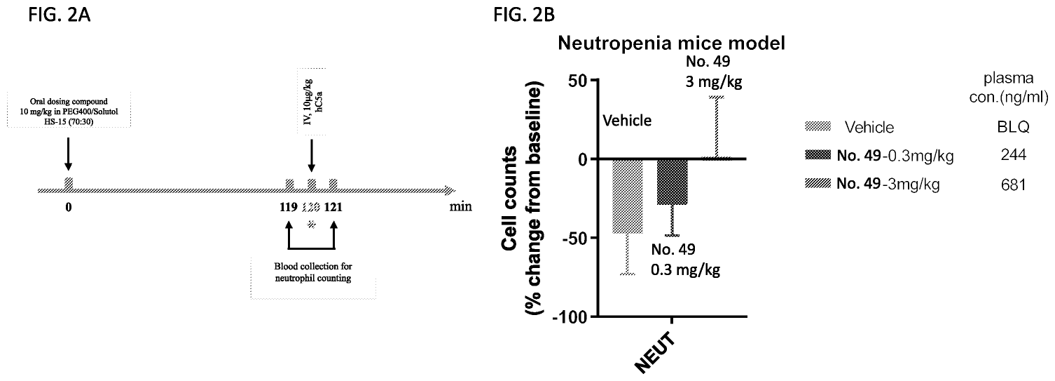

To study the efficacy of compounds in an animal model, human C5aR knock-in mice were created by replacing the coding region of mouse C5aR with human C5aR coding sequence. Intravenous injection of hC5a induced upregulation of adhesion molecules on blood vessel walls, leading to decreased neutrophils in the blood stream and attached to the vascular walls. Human C5aR knock-in mice were pre-dosed with vehicle or specific compound, and 2 hours later, human C5a (20 g/kg, ACROBiosystems) was administrated and 1 minute later neutrophils are quantified in peripheral blood. The experimental design is shown in FIG. 2A.

FIG. 2B shows that compound #49 effectively rescued the neutropenia induced by human C5a in human C5aR knock-in mice at 0.3 mg/kg and 3 mg/kg. The percent change in the number of neutrophils in the blood collected after C5a injection (121 min), relative to the sample collected prior to C5a injection (119 min). The plasma concentration of each compound is the average concentration prior to C5a injection of 3 individual mice.

### Neutrophil CD11b FACS Assay

Peripheral blood samples were collected from Cynomolgus monkey/huC5aR knock-in mice at indicated timepoints. 100 μl aliquots were mixed with a range of C5a concentrations and incubated at 37° C. for 30 min. Blood was cooled down on wet ice for at least 3 min, anti-CD11b monoclonal antibody (BD Biosciences) was added and incubated at 4° C. for 60 min. Red blood cells were lysed by adding erythrocyte lysis buffer (Solarbio) and incubated on ice for 10 min, white blood cells were washed by pre-cold PBS twice and suspended by 2% PFA/PBS buffer. Neutrophils were classified with flow cytometry by their forward/side-scatter properties and mean fluorescence intensity of anti-CD11b staining on cells was read by FACS (Beckman).

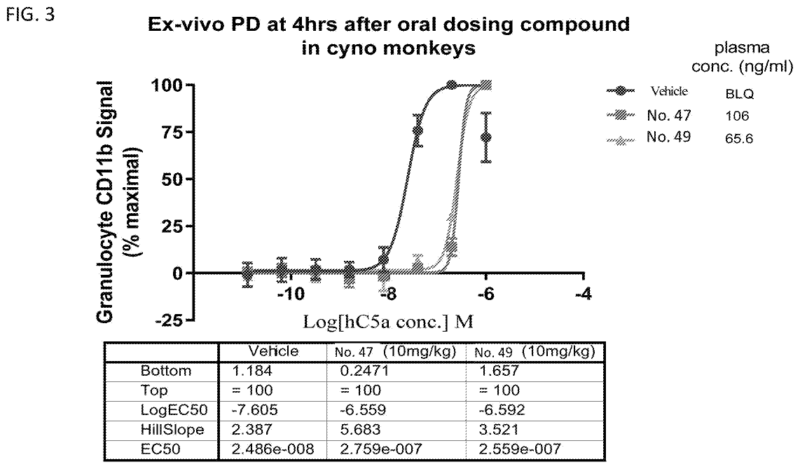

FIG. 3 shows that C5a induced CD11b upregulation on granulocytes ex-vivo in cyno monkey whole blood was blocked by orally pre-dosing compound Nos. 47 and 49 at 10 mg/kg. The EC50 values of compound No. 47 and 49 were comparable at 2.759×10⁻⁷ M and 2.559×10⁻⁷ M, respectively. The percent of CD11b signal was calculated by the formula %=(MFI[C5a conc.]-MFI[C5a=0])/(MFI[max]-MFI[C5a=0])×100%. Each data point was the average of 2 individual monkeys. The plasma concentration of each compound is the average concentration prior to C5a injection of 2 individual monkeys.

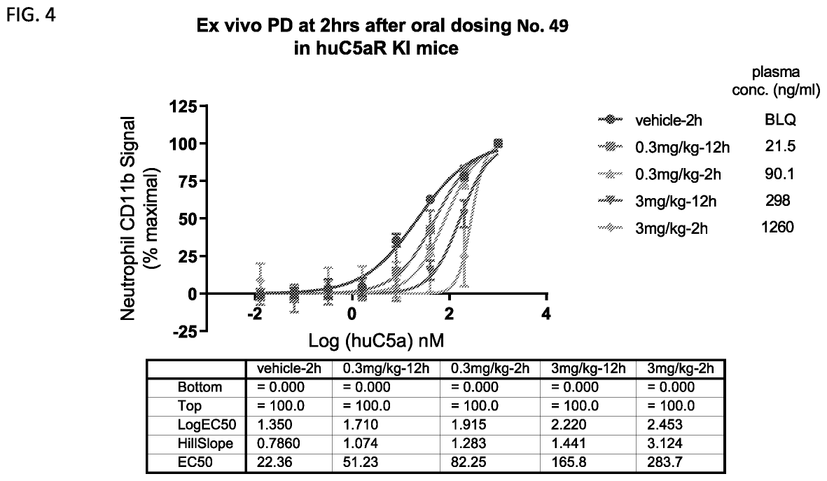

FIG. 4 shows that CD11b upregulation on neutrophil was blocked by orally pre-dosing compound No. 49 in mice whole blood at 0.3 mg/kg and 3 mg/kg, at 2 hours and 12 hours post-dosing. Whole blood was collected at indicated timepoints (2 hours or 12 hours after dosing compound) and further stimulated with human C5a in vitro. CD11b FACS antibody was added and incubate for 60 min at 4 C before red blood cells were lysed. The percent of CD11b signal was calculated by the formula %=(MFI[C5a conc.]-MFI[C5a=0])/(MFI[max]-MFI[C5a=0])×100%. Each data point was the average SD of 3 individual mice.

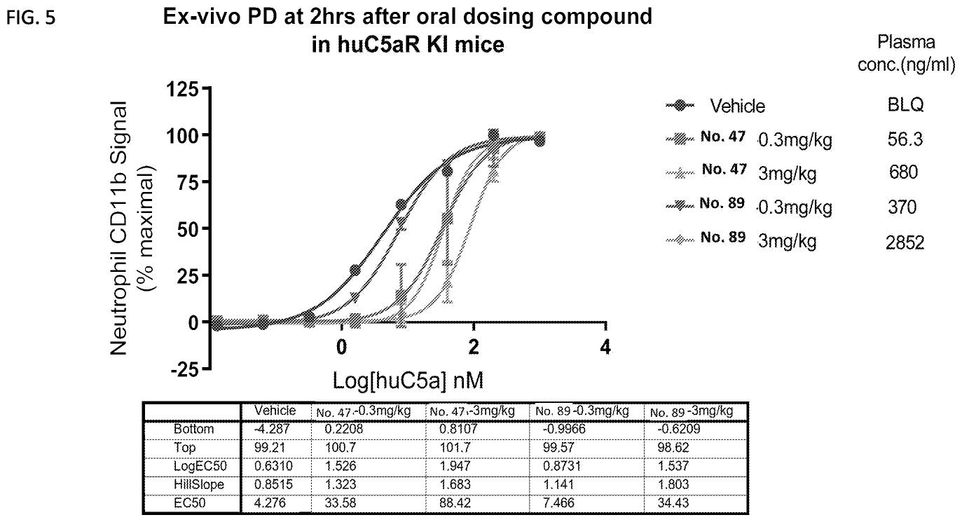

FIG. 5 shows that CD11b upregulation on neutrophil was blocked by orally pre-dosing compound Nos. 47 and 89 in mice whole blood at 0.3 mg/kg and 3 mg/kg at 2 hours after dosing. Whole blood was collected at 2 hrs after dosing compound and further stimulated with human C5a in vitro. CD11b FACS antibody was added and incubate for 60 min at 4 C before red blood cells were lysed. The percent of CD11b signal was calculated by the formula %=(MFI[C5a conc.]-MFI[C5a=0])/(MFI[max]-MFI[C5a=0])×100%. Each data point was the average of 2 individual mice.

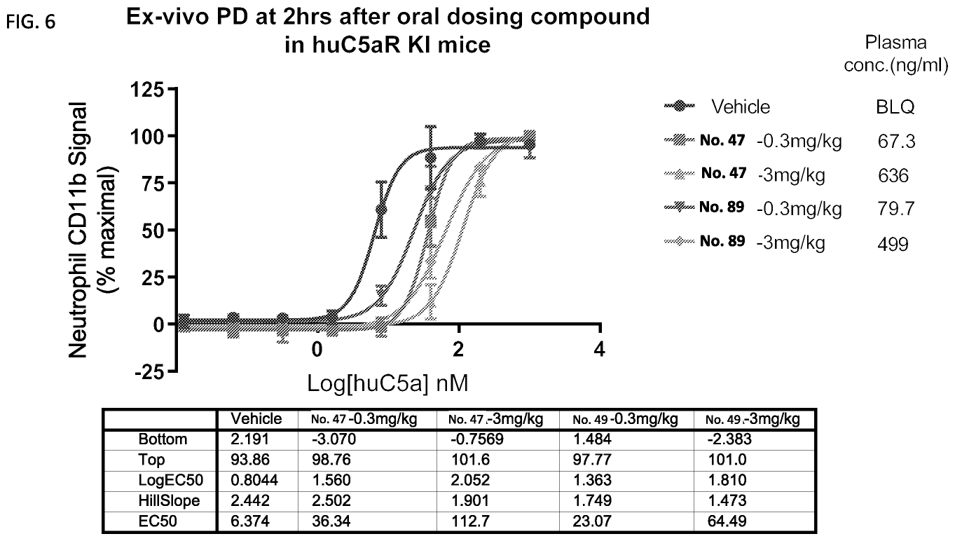

FIG. 6 shows that CD11b upregulation on neutrophil was blocked by orally pre-dosing compound Nos. 47 and 49 in mice whole blood at 0.3 mg/kg and 3 mg/kg at 2 hours after dosing. Whole blood was collected at 2 hrs after dosing compound and further stimulated with human C5a in vitro. CD11b FACS antibody was added and incubate for 60 min at 4 C before red blood cells were lysed. The percent of CD11b signal was calculated by the formula %=(MFI[C5a conc.]-MFI[C5a=0])/(MFI[max]-MFI[C5a=0])×100%. Each data point was the average±SD of 2 individual mice.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

1. A compound of formula (I), 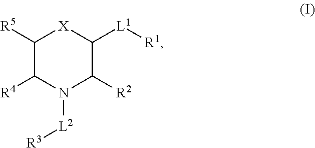
or a stereoisomer, tautomer, or a pharmaceutically acceptable salt of any of the foregoing, wherein:
X is —O— or —CHR⁶—,
provided that when X is —O—, then L¹ is *—C(O)NH—** and L² is —C(O)—;
R¹ is C₃₋₁₂ cycloalkyl, 3- to 12-membered heterocyclyl, 5- to 12-membered heteroaryl, or C₆₋₁₄ aryl, each of which is independently optionally substituted with one or more R¹¹,
wherein each R¹¹ is independently oxo, C₁₋₆ alkyl, C₂₋₆ alkenyl, C₂₋₆ alkynyl, halogen, —CN, —OR^{1a}, —SR^{1a}, —NR^{1a}R^{1b}, —NO₂, —C(O)R^{1a}, —OC(O)R^{1a}, —C(O)OR^{1a}, —C(O)NR^{1a}R^{1b}, —OC(O)NR^{1a}R^{1b}, —NR^{1a}C(O)R^{1b}, —NR^{1a}C(O)OR^{1b}, —S(O)R^{1a}, —S(O)₂R^{1a}, —NR^{1a}S(O)R^{1b}, —C(O)NR^{1a} S(O)R^{1b}, —NR^{1a}S(O)₂R^{1b}, —C(O)NR^{1a}S(O)₂R^{1b}, —S(O)NR^{1a}R^{1b}, —S(O)₂NR^{1a}R^{1b}, —P(O)R^{1a}R^{1b}, C₃₋₆ cycloalkyl, 3- to 12-membered heterocyclyl, 5- to 12-membered heteroaryl, C₆₋₁₄ aryl, —(C₁₋₆ alkylene) NR^{1a}R^{1b}, —(C₁₋₆ alkylene) C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN,
wherein R^{1a} and R^{1b} are each independently H, C₁₋₆ alkyl, C₂₋₆ alkenyl, C₂-6 alkynyl, C₃₋₁₂ cycloalkyl, 3- to 12-membered heterocyclyl, 5- to 12-membered heteroaryl, or C₆₋₁₄ aryl, or
R^{1a} and R^{1b} are taken together with the nitrogen atom to which they attach to form a 3- to 12-membered heterocyclyl, which is optionally substituted with one or more substituents selected from the group consisting of C₁₋₆ alkyl, C₂-6 alkenyl, C₂₋₆ alkynyl, halogen, hydroxyl, C₁₋₆ alkoxy, and —CN;
R² is C₃₋₁₂ cycloalkyl, 3- to 12-membered heterocyclyl, or C₆₋₁₄ aryl, each of which is independently optionally substituted with one or more -Q-W, wherein:
Q is C₁-6 alkylene, —(N-L³-R^{Q})— or —O—,
wherein R^{Q} is H, C₁₋₆ alkyl, 5- to 12-membered heteroaryl, or C₆₋₁₄ aryl, and
L³ is —C(O)—, *—C(O)O—CH₂—**, or a bond, wherein * indicates the point of attachment to N and ** indicates the point of attachment to R^{Q}, W is H, C₃₋₁₂ cycloalkyl, 3- to 12-membered heterocyclyl, 5- to 12-membered heteroaryl, or C₆₋₁₄ aryl, each of which is independently optionally substituted with one or more R⁷;
R³ is H, C₃₋₁₂ cycloalkyl, 3- to 12-membered heterocyclyl, 5- to 12-membered heteroaryl, or C₆₋₁₄ aryl, wherein the C₃₋₁₂ cycloalkyl, 3- to 12-membered heterocyclyl, 5- to 12-membered heteroaryl, and C₆₋₁₄ aryl are each independently optionally substituted with one or more R³¹
wherein each R³¹ is independently oxo, C₁₋₆ alkyl, C₂₋₆ alkenyl, C₂₋₆ alkynyl, halogen, —CN, —OR^{3a}, —SR^{3a}, —NR^{3a}R^{3b}, —NO₂, —C(O)R^{3a}, —OC(O)R^{3a}, —C(O)OR^{3a}, —C(O)NR^{3a}R^{3b}, —OC(O)NR^{3a}R^{3b}, —NR^{3a}C(O)R^{3b}, —NR^{3a}C(O)OR^{3b}, —S(O)R^{3a}, —S(O)₂R^{3a}, —NR^{3a}S(O)R^{3b}, —C(O)NR^{3a} S(O)R^{3b}, —NR^{3a}S(O)₂R^{3b}, —C(O)NR^{3a}S(O)₂R^{3b}, —S(O)NR^{3a}R^{3b}, —S(O)₂NR^{3a}R^{3b}, —P(O)R^{3a}R^{3b} C₃₋₆ cycloalkyl, 3- to 12-membered heterocyclyl, 5- to 12-membered heteroaryl, C₆₋₁₄ aryl, —(C₁₋₆ alkylene) NR^{3a}R^{3b}, —(C₁₋₆ alkylene)C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁-6 alkoxy, and —CN,
wherein R^{3a} and R^{3b} are each independently H, C₁₋₆ alkyl, C₂₋₆ alkenyl, C₂-6 alkynyl, C₃₋₁₂ cycloalkyl, 3- to 12-membered heterocyclyl, 5- to 12-membered heteroaryl, or C₆₋₁₄ aryl, or
R^{3a} and R^{3b} are taken together with the nitrogen atom to which they attach to form a 3- to 12-membered heterocyclyl, which is optionally substituted with one or more substituents selected from the group consisting of C₁₋₆ alkyl, C₂-6 alkenyl, C₂₋₆ alkynyl, halogen, hydroxyl, C₁₋₆ alkoxy, and —CN; and
R⁴, R⁵, and R⁶ are each independently H, C₁₋₆ alkyl, C₂₋₆ alkenyl, C₂₋₆ alkynyl, halogen, —CN, hydroxyl, C₁₋₆ alkoxy, C₃₋₆ cycloalkyl, 3- to 12-membered heterocyclyl, 5- to 12-membered heteroaryl, or C₆₋₁₄ aryl, wherein the C₁₋₆ alkyl, C₂₋₆ alkenyl, C₂₋₆ alkynyl, C₁₋₆ alkoxy, C₃₋₆ cycloalkyl, 3- to 12-membered heterocyclyl, 5- to 12-membered heteroaryl, and C₆₋₁₄ aryl are each independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN, and wherein,
R⁴ and R⁵ or R⁵ and R⁶ may be taken together with the carbon atoms to which they are attached to form a ring B which is independently optionally substituted with one or more R⁸, wherein ring B is C₃₋₁₂ cycloalkyl or 3- to 12-membered heterocyclyl, and
R⁴ may be taken with the carbon atom to which it is attached, the nitrogen atom adjacent to the carbon atom, L², and part of R³ to form a 6- to 8-membered heterocyclyl;
each R⁷ is independently oxo, C₁₋₆ alkyl, C₂₋₆ alkenyl, C₂₋₆ alkynyl,
halogen, —CN, —OR^{7a}, —SR^{7a}, —NR^{7a}R^{7b}, —NO₂, —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR⁷aS(O)R^{7b}, —C(O)NR⁷a S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)R^{7a}R^{7b}, C₃₋₆ cycloalkyl, 3- to 12-membered heterocyclyl, 5- to 12-membered heteroaryl, C₆₋₁₄ aryl, —(C₁₋₆ alkylene) NR^{7a}R^{7b}, —(C₁₋₆ alkylene)C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN, wherein
R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 12-membered heteroaryl, or C₆₋₁₄ aryl, or
R^{7a} and R^{7b} are taken together with the nitrogen atom to which they attach to form a 3- to 12-membered heterocyclyl, which is optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN;
each R⁸ is independently oxo, C₁₋₆ alkyl, C₂₋₆ alkenyl, C₂₋₆ alkynyl,
halogen, —CN, —OR^{8a}, —SR^{8a}, —NR^{8a}R^{8b}, —NO₂, —C(O)R^{8a}, —OC(O)R^{8a}, —C(O)OR^{8a}, —C(O)NR^{8a}R^{8b}, —OC(O)NR^{8a}R^{8b}, —NR^{8a}C(O)R^{8b}, —NR^{8a}C(O)OR^{8b}, —S(O)R^{8a}, —S(O)₂R^{8a}, —NR^{8a}S(O)R^{8b}, —C(O)NR^{8a} S(O)R^{8b}, —NR^{8a}S(O)₂R^{8b}, —C(O)NR^{8a}S(O)₂R^{8b}, —S(O)NR^{8a}R^{8b}, —S(O)₂NR^{8a}R^{8b}, —P(O)R^{8a}R^{8b}, C₃₋₆ cycloalkyl, 3- to 12-membered heterocyclyl, 5- to 12-membered heteroaryl, C₆₋₁₄ aryl, —(C₁₋₆ alkylene) NR^{8a}R^{8b}, —(C₁₋₆ alkylene)C₃₋₆ cycloalkyl, —(C₁₋₆ alkylene) 3- to 12-membered heterocyclyl, —(C₁₋₆ alkylene) 5- to 12-membered heteroaryl, or —(C₁₋₆ alkylene) C₆₋₁₄ aryl, each of which is independently optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN, wherein
R^{8a} and R^{8b} are each independently H, C₁₋₆ alkyl, C₂₋₆ alkenyl, C₂₋₆ alkynyl, C₃₋₁₂ cycloalkyl, 3- to 12-membered heterocyclyl, 5- to 12-membered heteroaryl, or C₆₋₁₄ aryl, or
R^{8a} and R^{8b} are taken together with the nitrogen atom to which they attach to form a 3- to 12-membered heterocyclyl, which is optionally substituted with one or more substituents selected from the group consisting of C₁₋₆ alkyl, C₂₋₆ alkenyl, C₂₋₆ alkynyl, halogen, hydroxyl, C₁₋₆ alkoxy, and —CN;
L¹ is *—C(O)NH—**, a bond, —C(O)—, *—CH₂—NH—**, or *—C(O)NH—CH₂—**, wherein * indicates the point of attachment to the carbon atom of the piperidine and ** indicates the point of attachment to R¹;
L² is —C(O)—, a bond, —CH₂—, —S(O)₂—, or #—S(O)₂—CH₂— ##, wherein #indicates the point of attachment to the nitrogen atom and ##indicates the point of attachment to R³,
provided that when X is —CHR⁶— and R⁴, R⁵, and R⁶ are all H, then at least one of the following conditions apply:
(1) L¹ is a bond, —C(O)—, *—CH₂—NH—**, or *—C(O)NH—CH₂—**
(2) L² is a bond, —CH₂—, —S(O)₂—, or #—S(O)₂—CH₂— ##, and
(3) R² is phenyl substituted with one or more -Q-W, wherein Q is —(N-L³-R^{Q})— and R^{Q} is 5- to 12-membered heteroaryl or C₆₋₁₄ aryl.

2. The compound claim 1, or a stereoisomer, tautomer, or a pharmaceutically acceptable salt of any of the foregoing, wherein R⁴ and R⁵ are taken together with the carbon atoms to which they are attached to form a ring B, which is optionally substituted with one or more R⁸.

3. The compound of claim 1, or a stereoisomer, tautomer, or a pharmaceutically acceptable salt of any of the foregoing, wherein ring B is a C₃₋₁₂ cycloalkyl, which is optionally substituted with one or more R⁸.

4. The compound of claim 3, or a stereoisomer, tautomer, or a pharmaceutically acceptable salt of any of the foregoing, wherein ring B is cyclopentyl, which is optionally substituted with one or more R⁸.

5. The compound of claim 1, or a stereoisomer, tautomer, or a pharmaceutically acceptable salt of any of the foregoing, wherein ring B is a 3- to 12-membered heterocyclyl, which is optionally substituted with one or more R⁸.

6. The compound of claim 5, or a stereoisomer, tautomer, or a pharmaceutically acceptable salt of any of the foregoing, wherein the ring B is tetrahydrofuranyl, which is optionally substituted with one or more R⁸.

7. The compound of claim 5, or a stereoisomer, tautomer, or a pharmaceutically acceptable salt of any of the foregoing, wherein B is pyrrolidinyl, which is optionally substituted with one or more R⁸.

8. The compound of claim 1, or a stereoisomer, tautomer, or a pharmaceutically acceptable salt of any of the foregoing, wherein each R⁸ is independently C₁₋₆ alkyl, C₃₋₆ cycloalkyl, 3- to 12-membered heterocyclyl, or —C(O)R^{8a}, each of which is independently optionally substituted with one or more halogen.

9. (canceled)

10. (canceled)

11. The compound of claim 1, or a stereoisomer, tautomer, or a pharmaceutically acceptable salt of any of the foregoing, wherein X is —CHR⁶—, wherein R⁶ is H.

12. (canceled)

13. The compound of claim 1, or a stereoisomer, tautomer, or a pharmaceutically acceptable salt of any of the foregoing, wherein R¹ is C₆₋₁₄ aryl, which is optionally substituted with one or more R¹¹.

14. The compound of claim 13, or a stereoisomer, tautomer, or a pharmaceutically acceptable salt of any of the foregoing, wherein R¹ is phenyl, which is optionally substituted with one or more R¹¹.

15-19. (canceled)

20. The compound of claim 1, or a stereoisomer, tautomer, or a pharmaceutically acceptable salt of any of the foregoing, wherein R¹ is selected from the group consisting of:

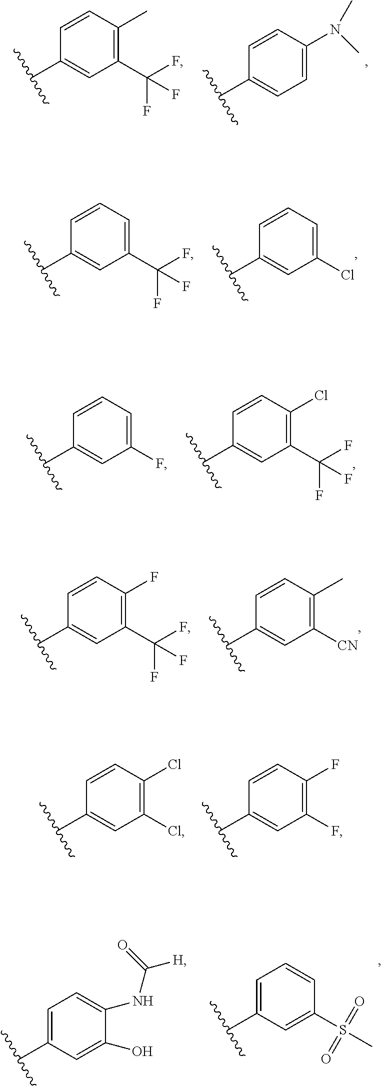

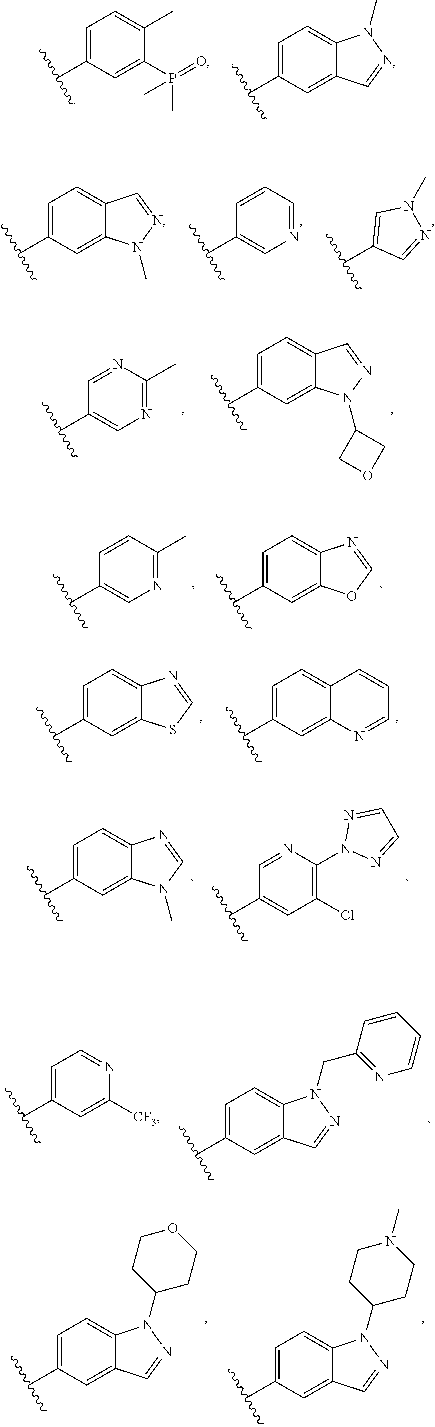

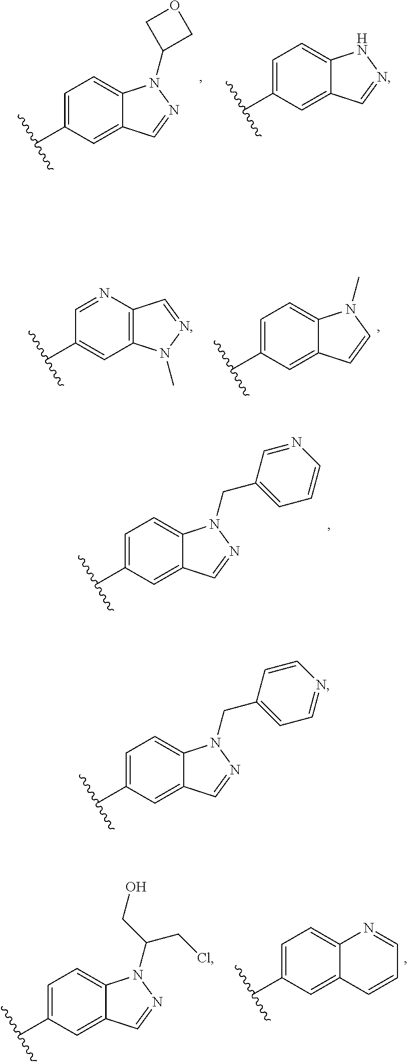

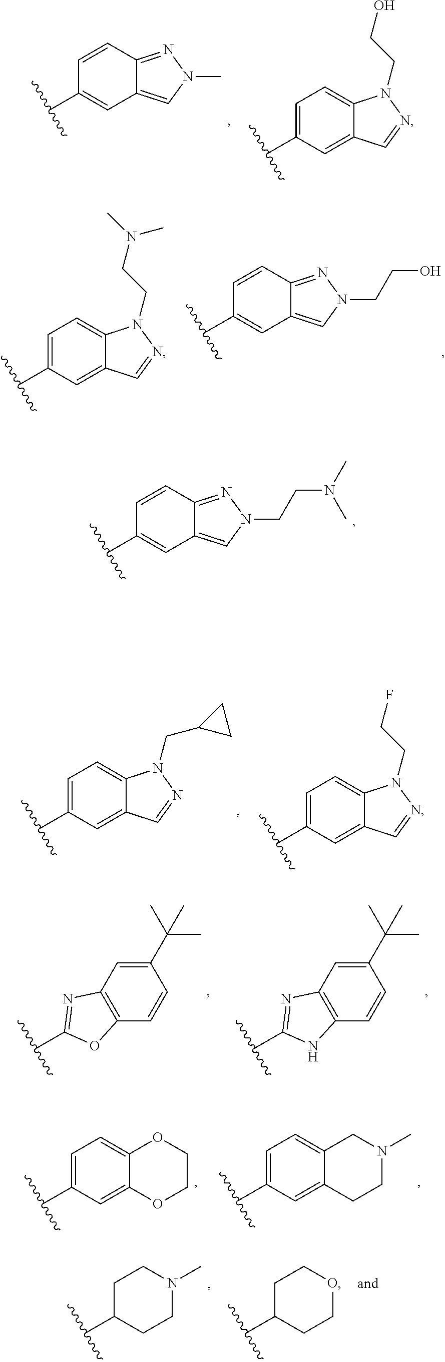

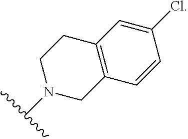 [SMILES: *N1CCc2cc(Cl)ccc2C1]

21. The compound of claim 1, or a stereoisomer, tautomer, or a pharmaceutically acceptable salt of any of the foregoing, wherein R² is C₆₋₁₄ aryl optionally substituted with one or more -Q-W.

22-26. (canceled)

27. The compound of claim 1, or a stereoisomer, tautomer, or a pharmaceutically acceptable salt of any of the foregoing, wherein Q is —(N-L³-R^{Q})—.

28. The compound of claim 27, or a stereoisomer, tautomer, or a pharmaceutically acceptable salt of any of the foregoing, wherein R^{Q} is H.

29. (canceled)

30. The compound of claim 1, or a stereoisomer, tautomer, or a pharmaceutically acceptable salt of any of the foregoing, wherein W is C₃₋₁₂ cycloalkyl, which is optionally substituted with one or more R⁷.

31-42. (canceled)

43. The compound of claim 1, or a stereoisomer, tautomer, or a pharmaceutically acceptable salt of any of the foregoing, wherein R³ is selected from the group consisting of:

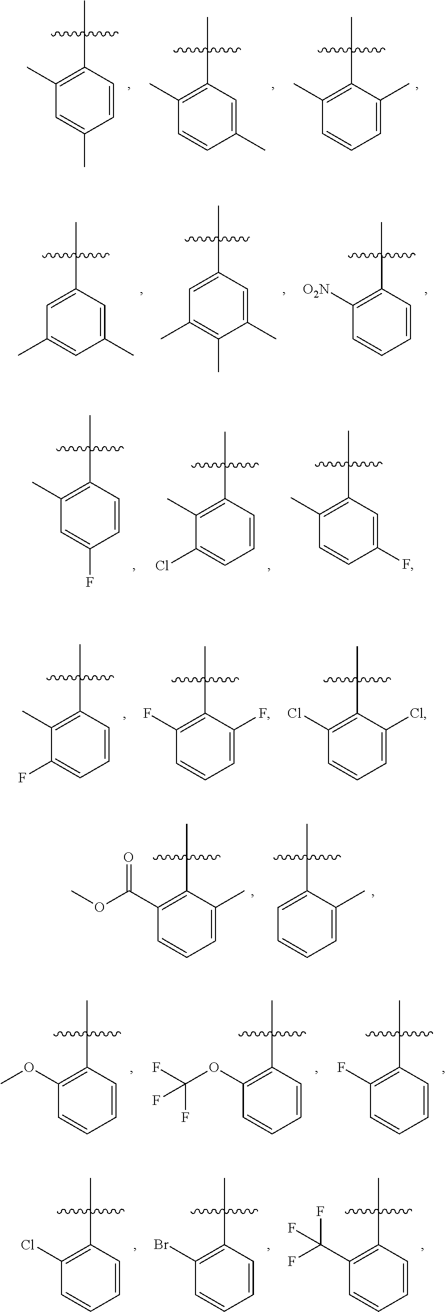

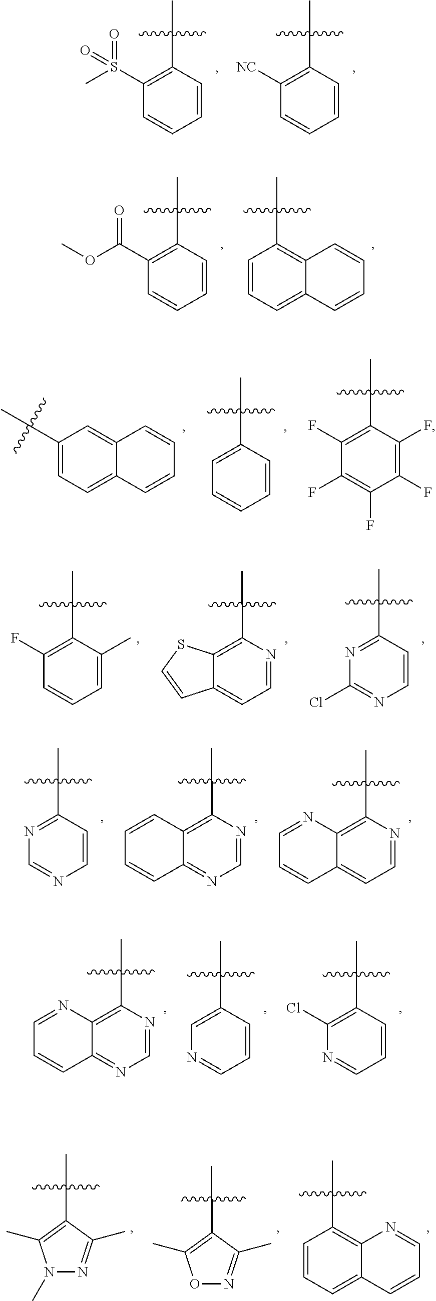

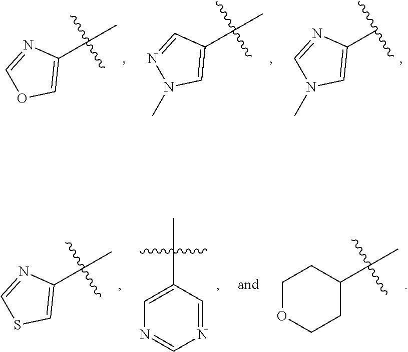

44-52. (canceled)

53. The compound of claim 1, where the compound is of formula (VIII),

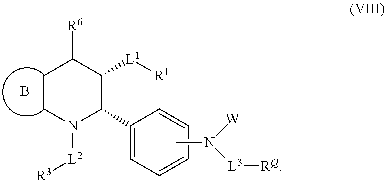

54. A compound selected from the group consisting of the compounds in Table 1, or a pharmaceutically acceptable salt of any of the foregoing.

55. A pharmaceutical composition comprising the compound of claim 1, or a stereoisomer, tautomer, or a pharmaceutically acceptable salt of any of the foregoing, and a pharmaceutically acceptable carrier or excipient.

56. (canceled)

57. A method of inhibiting C5a receptor, comprising contacting C5a receptor with a compound of claim 1, or a stereoisomer, tautomer, or a pharmaceutically acceptable salt of any of the foregoing.

58. A method of treating a disorder mediated by the complement pathways in a subject, comprising administering to the subject a therapeutically effective amount of a compound of claim 1, or a stereoisomer, tautomer, or a pharmaceutically acceptable salt of any of the foregoing.

59. The method of claim 58, wherein the disorder is an inflammatory disease, a cardiovascular or cerebrovascular disease, or an autoimmune disease.

60. The method of claim 59, wherein the disorder is an autoimmune disease.

61. The method of claim 58, wherein the disease or disorder is at least selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD), bullous pemphigoid, hidradenitis suppurativa, dermatitis herpetiformis, sweets syndrome, pyoderma gangrenosum, palmo-plantar pustulosis & pustular psoriasis, rheumatoid neutrophilic dermatoses, subcorneal pustular dermatosis, bowel-associated dermatosis-arthritis syndrome, neutrophilic eccrine hidradenitis, linear IgA disease, or any combinations thereof.
